(19) United States
(12) Patent Application Publication
(10) Pub. No.: US 2026/0218305 A1
(43) Pub. Date: Jul. 30, 2026

(54) NON-INVASIVE DISEASE DETECTION AND MONITORING

(71) Applicant: Cancer Research Technology Limited, London (GB)

(72) Inventors: Charles Massie, formerly of Cambridge (GB); Radoslaw Lach, Cambridge (GB); Harveer S. Dev, Cambridge (GB); Gahee Park, Cambridge (GB)

(73) Assignee: Cancer Research Technology Limited, London (GB)

(21) Appl. No.: 19/121,696

(22) PCT Filed: Oct. 17, 2023
(86) PCT No.: PCT/EP2023/078880

§ 371 Date: Apr. 16, 2025

(30) Foreign Application Priority Data
Oct. 17, 2022 (GB) 2215319.1

(51) Int. Cl. C12Q 1/6886; G16B 30/00

(52) U.S. Cl. CPC C12Q 1/6886; G16B 30/00; C12Q 2600/112; C12Q 2600/118; C12Q 2600/154

(57) ABSTRACT

The present invention provides a method of analysing a sample obtained from a subject having or suspected of having a somatic disease state, the method comprising: (a) providing a tissue, proximal or distal sample of limited quantity, such as a cell-free DNA (cfDNA)-containing sample obtained from the subject and performing methylation sequencing of at least 10 predetermined genomic regions, wherein said methylation sequencing may comprise a non-destructive, enzyme-based conversion method to differentiate unmethylated cytosine from 5-methylcytosine and/or 5-hydroxymethylcytosine; (b) generating metrics from the methylation sequencing reads in step (a) that represent one or both of (i) a measure of the degree to which the methylation observed within a sequencing read is similar to a previously determined disease methylation signal, said measure comprising a read rating generated by providing a vector of methylation states for each methylation sequencing read and summing the information content of all methylation states that are concordant with the methylation state for the same region previously determined in a one or more disease samples; and (ii) a measure of intra-read methylation heterogeneity; and (c) integrating the metrics generated in step (b) across the at least 10 predetermined genomic regions to obtain a global measure of disease-originating cfDNA in the sample obtained from the subject. Related methods and systems for use in performing the methods are also provided.

This application claims priority from GB2215319.1 filed 17 Oct. 2022, the contents and elements of which are herein incorporated by reference for all purposes.

### FIELD OF THE DISCLOSURE

The present invention relates to methods for detecting signal in samples such as limited input samples and liquid biopsy cell-free DNA (cfDNA) samples, for somatic diseases states that are characterised by alterations of epigenetic markers in cfDNA, such as cancer. Methods for analysing a sample, detecting and/or staging a cancer, monitoring cancer progression or remission, or monitoring response to an anti-cancer therapy in said subject are provided as are related computer-implemented methods and systems.

### BACKGROUND

Cell-free tumour DNA (ctDNA) analysis has been adopted as a methodology for companion diagnostics and monitoring treatment response, in the management of late-stage metastatic cancers [1-3]. The minimally invasive process for obtaining blood, other body fluids or proximal samples, has paved the way for molecular profiling and disease burden monitoring in groups of patients that would not routinely have tumour tissue biopsies. The success of ctDNA mutation profiling in metastatic patients has initiated many exploratory studies aiming to detect early-stage cancers and minimal residual disease following radical treatment. Early data suggests that ctDNA analysis can pick up a subset of patients that relapse after treatment with curative intent and may help to stratify these patients for second line treatments [4-6]. However, early detection of cancer remains an unsolved challenge and only ~40% of stage-I cancers have been detected using high-sensitivity genomics methods combined with tumour-specific mutations [7-9].

One of the major limitations for ctDNA analysis in low burden disease (such as early-stage cancers) is the low total number of genomic copies that can be isolated from blood plasma (~10³ copies per ml blood plasma) and the low fraction originating from diseased cells (commonly <0.1% of the total cfDNA fraction). This results in hard sampling limits that constrain the analytical sensitivity of single-locus assays to parts-per-thousand [10-12], even when using experimental methods with technical detection limits in parts-per-million [9,13]. Recent ctDNA studies have proposed monitoring multiple mutations in parallel to improve analytical sensitivity [4,14] and one group has suggested monitoring thousands of patient-specific mutations to provide high analytical sensitivity with detection limits in parts-per-million [12]. However, each of these approaches requires a priori knowledge of the mutated sites in a given patient sample, either through the analysis of recurrently mutated genes (restricted to a small number of genes in certain cancers) or patient-specific mutations (where tumour tissue has previously been analysed). Therefore, these approaches have limited applicability for the early detection of diseases with a low number of recurrent mutations (e.g. most cancer types) and incur a significant investment of time and resources to profile each patient's tumour tissue to allow detection of MRD and treatment response monitoring

Previous studies have analysed the epigenome using bisulfite sequencing of cfDNA samples, focusing on small numbers of disease-specific markers [19-23] or global metrics of epigenetic change [18, 24-25]. Most recently, two academic groups and one large biotech company have sought to discover genome-wide features directly from plasma cfDNA analysis [26-30].

WO2021/146570 describes methods for diagnosing hepatocellular carcinoma. This reference is said to provide methylated cell-free DNA biomarkers and methods of using them to determine if a patient has hepatocellular carcinoma. Additionally, the methylated cell-free DNA biomarkers can be used to distinguish between patients with a chronic liver disease such as cirrhosis who do not have hepatocellular carcinoma and those patients with a chronic liver disease who have hepatocellular carcinoma.

Li et al., Cell Research, 2017, Vol. 27, pp. 1243-1257 (doi: 10.1038/cr.2017.121) describes 5-Hydroxymethylcytosine signatures in circulating cell-free DNA as diagnostic biomarkers for human cancers.

Rossi, S. H. (2022). Characterising DNA methylation in tissue and liquid samples from patients with renal tumours. https://doi.org/10.17863/CAM. 87636 describes characterisation of DNA methylation in tissue and liquid samples derived from patients with renal tumours.

However, despite these advances, there remains an unmet need for improved detection of somatic disease states, such as cancer. In particular, it would be desirable to develop more sensitive and/or specific methods for detecting ctDNA that occurs at low levels in liquid biopsy samples. The present invention aims address these unmet needs and provide related advantages.

### SUMMARY OF THE DISCLOSURE

Broadly, the present inventors have developed an improved method for detecting disease states affecting somatic cells that are characterised by alterations of epigenetic markers in cfDNA, such as cancer. This increased sensitivity of detection is achieved by undertaking a broader analysis of disease-specific features that can with improved experimental and be measured in cfDNA, combined computational workflows that maximise the recovery of disease markers at low levels and minimise background signals observed in unaffected individuals. In certain embodiments the method may be termed “NIMBUS”: Non-destructive integration of methylation to boost underlying signals. The present inventors have defined thousands of tumour-specific DNA methylation markers that can be analysed in parallel to improve ctDNA detection in low burden samples (see for example Tables 1-4 and 1A-4A, 6 and 6A herein). An optimised workflow has also been developed that permits recovery of a larger number of intact cfDNA molecules for ctDNA methylation analysis. The present inventors have also developed improved analytical methods by creating a global measure of ctDNA levels by integrating signals across 10²-10⁵ tumour-specific markers, each containing 21 informative molecule per locus above background signal in disease-free controls and with a combined minimum signal of 10-10⁴ informative molecules across all markers.

In comparison with the method described in Rossi, S. H. (2022), the method of the present differs in, at least, the following significant respects:

Whereas the pipeline of the method described in Rossi involves calculating a score similar to a likelihood, the present invention—in certain embodiments—employs the information content of the read to calculate scores (“read rating” as defined further herein). “Likelihood” as discussed in Rossi implies using probabilities of observation of given event. Moreover, the method of the present invention—in certain embodiments—employs thresholds for the loci/CpG that only contain rare information. Without wishing to be bound by any particular theory, the present inventors believe that the thresholds for the loci/CpG that only contain rare information leads to a clearer signal in comparison to the method of Rossi.

Further differences of note are that the method of Rossi categorises a read as tumour driven by comparing the distribution of methylation levels in the sample compared to the background. The method of the present invention—in certain embodiments—employs not the methylation levels for comparison, but the distribution of scores (read ratings) to decide whether a read is tumour-derived or not. Rossi, S. H. (2022) teaches that scores per read were summarised into an overall score per patient by adding the log of the individual read level scores (see Rossi page 199). Conversely, in accordance with the method of the present invention—in certain embodiments—the scores per read are not simply added. Rather, only the number of reads with particularly high scores are added (in this context, “particularly high scores” may be taken to mean those in the 99^{th} percentile or the 99.9^{th} percentile).

Accordingly, in a first aspect the present invention provides a method of analysing a sample obtained from a subject having or suspected of having a disease affecting somatic cells that can be detected through alterations in epigenetic cfDNA markers (e.g. a cancer), the method comprising:
(a) providing a cell-free DNA (cfDNA)-containing sample obtained from the subject and performing methylation sequencing of at least 10, 25, 50 or at least 100 predetermined genomic regions;
(b) generating metrics from the methylation sequencing reads in step (a) that represent one or both of (i) a measure of the degree to which the methylation observed within a sequencing read is similar to a previously determined disease methylation signal in cfDNA, said measure comprising a read rating generated by providing a vector of methylation states for each methylation sequencing read and summing the information content of all methylation states that 10 are concordant with the methylation state for the same region previously determined in a one or more disease samples; and (ii) a measure of intra-read methylation heterogeneity; and
(c) integrating the metrics generated in step (b) across the at least 10, 25, 50 or the at least 100 predetermined genomic regions to obtain a global measure of disease-originating cfDNA in the sample obtained from the subject.

In some embodiments, said methylation sequencing may comprise a non-destructive, enzyme-based conversion method to differentiate unmethylated cytosine from 5-methylcytosine and/or 5-hydroxymethylcytosine. In some embodiments, said methylation sequencing may comprise a step of bisulphite conversion (sometimes known as “bisulphite sequencing”). In some embodiments, said methylation sequencing may comprise direct measurement of base modifications, e.g., on a single molecule sequencing platform, such as via nanopore sequencing platforms or PacBio sequencing platforms.

In some embodiments the disease characterised by somatic alterations in the genome and epigenome comprises a neoplasm, such as a tumour. The subject may have a malignant tumour, which sheds tumour-derived DNA into one or more bodily fluids, such as blood plasma. In cases where the disease state is a cancer, the global measure of disease-originating cfDNA in the sample may be a measure of circulating tumour DNA (ctDNA) in the sample obtained from the subject. The present invention finds use, in particular, in detection of ctDNA methylation signal even in low burden samples, such as small volume plasma samples (containing relatively low levels (absolute amount or tumour fraction) of ctDNA) or samples obtained from a subject with early-stage disease or at an early time point in disease recurrence.

In some embodiments each of said at least 10 predetermined genomic regions satisfies at least two (preferably all three) of the following conditions:
(i) the region (which may be termed a “marker region”) comprises a differentially methylated cytosine (DMC) or differentially methylated region (DMR) defined by the methylation status of one or more disease-state specific CpG, wherein said DMC/DMR exhibits a significance threshold of q<0.05 or q<0.01 for a high-burden disease sample (e.g. cancer tissue, proximal sample or high disease burden cfDNA sample) versus healthy control tissue (and/or healthy control cfDNA) as determined by differential methylation analysis (e.g. MethylKit [Akalin, A., Kormaksson, M., Li, S. et al. methylKit: a comprehensive R package for the analysis of genome-wide DNA methylation profiles. Genome Biol 13, R87 (2012). doi.org/10.1186/gb-2012-13-10-r87])
(ii) CpGs within the region exhibit a difference in methylation between healthy cfDNA samples and disease tissue (e.g. cancer tissue) within the range 0.6 to 1.0, optionally wherein the difference is determined according to the formula |betaₚₗₐₛₘₐ−round(betaₜᵢₛₛᵤₑ)>0.6;
(iii) the region comprises at least 2, at least 5 or at least 10 CpGs.

In some embodiments the at least 10 predetermined genomic regions comprises at least 25, at least 50, at least 100, at least 500, at least 1000, at least 5000 or at least 10000 predetermined genomic regions. In certain embodiments, the predetermined regions do not extend to the entire genome or entire exome of the subject. By selecting informative regions and integrating the disease-originating methylation signal across those regions, embodiments of the present invention were found to strike a balance between sequencing resource and sensitivity for detecting the disease-originating signal. The predetermined regions may, in some cases, be selected from the genomic regions set forth in any one of Tables 1 to 4 or 1A to 4A or 6 or 6A herein. In some embodiments, the number of predetermined genomic regions employed and/or number of CpGs within the predetermined genomic regions employed does not exceed 1 million (e.g. is less than 100,000). In particular, using fewer predetermined genomic regions and focusing on those regions which are informative in the sense of providing disease vs. non-disease distinguishing signal, resources (such as sequencing resources and/or computational resources) may be spared. In particular, employing well-chosen regions in this way contrasts with a more resource-intensive methods such as those employing whole genome methylation sequencing. In some embodiments, at least 10%, at least 25%, at least 50%, at least 75% or at least 90% of the predetermined genomic regions employed in the method of any aspect of the invention are selected from those set forth in any one of Tables 1~4 or 6 or Tables 1A-4A or 6A.

In some embodiments, the metric that measures the degree to which the methylation observed within a sequencing read is similar to a previously determined disease methylation signal (e.g. cancer methylation signal), comprises a “read rating” (also known as rare read rating) generated by providing a vector of methylation states for each methylation sequencing read and summing the information content of all methylation states that are concordant with the methylation state for the same region previously determined in a disease tissue sample or other high disease burden sample (e.g. a cancer tissue sample, proximal sample or high-burden distal sample). In some cases “concordant with the methylation state for the same region previously determined in a disease sample” means a consensus methylation state determined in cohorts of at least 50 (e.g between 50 and 500) disease tissue or other high disease burden samples compared to disease-free control samples. In some embodiments, the read rating is a penalised read rating, the generation of which further comprises subtracting the information content of all of the methylation states opposite to the methylation state for the same region previously determined in the disease sample (e.g. a cancer tissue sample).

In some embodiments the read rating for each sequencing read is compared with a read rating threshold to determine whether the read is a disease-specific read (e.g. a cancer-specific read), optionally after correcting for background methylation for the same region in samples from healthy controls. The read rating threshold may be a constant value (e.g. zero Shannons or 4.32 Shannons) or a statistical measure of variation from a distribution of read ratings observed for the same region in healthy controls (e.g. the 99^{th} quantile or two or three standard deviations above the mean for healthy controls, or a z-score of 3 or more).

In some embodiments integrating the metrics generated in step (b) across the at least 100 predetermined genomic regions to obtain the global measure comprises summing all the read ratings for all sequencing reads that pass the read rating threshold to be determined as disease-specific reads (e.g. cancer-specific reads). By integrating or aggregating the read rating metrics in this way, embodiments of the present invention are able to detect disease-originating methylation signals that would not be apparent when focusing on, e.g., a single somatic mutation (c.f. FIGS. 1 and 2).

In some embodiments the metric that measures intra-read methylation heterogeneity, comprises a simple methylation uniformity gauge (SMUGE) score. The SMUGE score may be generated by providing a vector \vec{rₖ} of methylation states for each methylation sequencing read, wherein

$$\vec{{r}_{k}} = [ {r}_{1} , … , {r}_{j} ] , where {r}_{i} ∈ ( - 1 , 1 )$$

where −1 is unmethylated cytosine and 1 is methylated cytosine in a CpG context, j is the number of methylation sites in a read from the predetermined genomic region and wherein the SMUGE score for the k^{th} read is determined according to the formula:

$${smuge}_{k} = {\sum }_{j}^{J - 1} \frac{\vert {r}_{j} - {r}_{j + 1} \vert}{2}$$

In some embodiments integrating the metrics generated in step (b) across the at least 10 predetermined genomic regions to obtain the global measure comprises summing or averaging the SMUGE scores for all sequencing reads that pass the SMUGE threshold to be determined as disease-specific reads (e.g. cancer-specific reads). By integrating or aggregating the SMUGE metrics in this way, embodiments of the present invention are able to detect disease-originating methylation signals that would not be apparent when focusing on, e.g., a single somatic mutation (c.f. FIG. 1 versus FIG. 2b).

In some embodiments, the two metrics are combined and/or utilised together in order to better distinguish disease-originating reads from reads that originate from healthy sources. For example, the metrics that represent (i) the measure of the degree to which the methylation observed within a sequencing read is similar to a previously determined cancer methylation signal; and (ii) the measure of intra-read methylation heterogeneity may be combined into a single score. In some embodiments, the measure of intra-read methylation heterogeneity (e.g. the SMUGE score) is used to classify reads that cannot be unequivocally classified using metric (i) (e.g. read rating) alone. In this embodiment, the method may involve a decision tree approach in which a read is classified as clearly disease, clearly non-disease or uncertain based on the read rating metric. For reads classified as uncertain, the second metric (e.g. SMUGE score) may be used to classify as disease or non-disease originating. Similarly, in some cases, the SMUGE score may be used to classify reads as originating from a disease source of a particular disease stage (e.g. cancer stage) or as originating from a metastatic cancer. An example of this approach as applied to prostate cancer staging is shown in FIG. 8.

In some embodiments of the present invention the cell-free DNA (cfDNA)-containing sample comprises 1 μg (i.e. picogram) to 1 μg, such as 1 μg to 200 ng or 1 ng to 200 ng of cfDNA and/or wherein the cfDNA sample comprises less than 10000, less than 1000, less than 300 or less than 100 haploid human genome equivalents. The cfDNA sample may be subjected to amplification and/or library preparation prior to methylation sequencing. In some embodiments the library preparation may be single stranded DNA (ssDNA) library preparation (FIGS. 3 and 4).

In some embodiments the non-destructive, enzyme-based conversion method comprises ten-eleven translocation dioxygenase 2 (TET2)-based conversion. The TET2-based conversion may be followed by a bead-based clean-up step of the TET2-converted DNA in which the bead ratio is selected to enhance recovery of total cfDNA, resulting in enhanced detection of rare signals (e.g. low fraction ctDNA, FIG. 4).

In some embodiments the methylation sequencing comprises targeted capture sequencing. The targeted capture sequencing may comprise a preceding step of PCR amplification of the post-conversion cfDNA using uracil base-tolerant PCR reagents. In particular, the uracil base-tolerant PCR reagents may comprise the KAPA HiFi HotStart Uracil+ReadyMix (Roche), resulting in more efficient sequencing library production and increased sample complexity for the detection of rare signals (e.g. low fraction ctDNA, FIG. 4).

In some embodiments the targeted capture sequencing comprises a target enrichment step that employs one or more probe panels that target said at least 10 predetermined genomic regions. The one or more probe panels may target at least 10, at least 25, at least 50, at least 100, at least 1000, at least 5000 or at least 10000 genomic regions set forth in any one of Tables 1 to 4 or 6 or Tables 1A to 4A or 6A.

In some embodiments the method comprises a step, preceding said non-destructive, enzyme-based conversion, in which at least one biotinylated adaptor is ligated to cfDNA in said cfDNA-containing sample (FIG. 5a). This allows solid-phase bead-coupling throughout the non-destructive conversion workflow, in order to minimise sample loss during the multiple protocol steps (‘clean-up’) and maintain sample complexity. As shown in FIG. 5b, addition of biotinylated adaptors was found to increase the number of informative reads (i.e. the proportion of reads used by the algorithm in the NIMBUS method). This in turn led to improved assay performance (see FIGS. 19A and 19B).

In some embodiments the method is for detecting and/or staging a cancer, monitoring cancer progression or remission, or monitoring response to an anti-cancer therapy in said subject. For example, detection of a ctDNA methylation signal in sample obtained from a subject suspected of having a cancer may provide a diagnostic aid or confirm the presence of cancer. Additionally or alternatively, the NIMBUS method of the present invention may find use in the detection of minimal residual disease (MRD) (e.g. following surgical excision of a tumour) or recurrence of a cancer following a course of treatment (e.g. chemotherapy, radiotherapy, immunotherapy, targeted therapy and/or surgery).

In some embodiments the method of the present invention may be carried out on at least one sample obtained from each of a plurality of subjects having or suspected of having cancer (or other disease states). The method may therefore find use in stratifying the plurality of subjects according to the stage, prognosis or future risk of progression of the cancer (or other disease states).

In some embodiments of the present invention the cfDNA-containing sample may comprise a tissue sample (e.g. biopsy), proximal sample (e.g. urine, saliva) or a minimally invasive distal sample (e.g. plasma or other blood sample).

In some embodiments the somatic disease state may be a neoplasm such as prostate cancer (PrCa). For example, early stage (localised PrCa), late stage (locally advanced PrCa) or metastatic PrCa. The at least 10 predetermined genomic regions may be selected from the PrCa genomic regions set forth in Table 4 or Table 4A or Table 6 or Table 6A. For example, the predetermined genomic regions may comprise at least 10%, 25%, 50%, 75% or at least 90% of (or even all of) the genomic regions set forth in Table 4 or Table 4A or Table 6 or Table 6A.

In some embodiments the somatic disease state may be oesophageal carcinoma (OAC) (also written as esophageal carcinoma (EAC)). The at least 10 predetermined genomic regions may be selected from the OAC genomic regions set forth in Table 3 or Table 3A. For example, the predetermined genomic regions may comprise at least 10%, 25%, 50%, 75% or at least 90% of (or even all of) the genomic regions set forth in Table 3 or Table 3A.

In some embodiments the somatic disease state may be glioblastoma (GBM). The at least 10 predetermined genomic regions may be selected from the GBM genomic regions set forth in Table 2 or Table 2A. For example, the predetermined genomic regions may comprise at least 10%, 25%, 50%, 75% or at least 90% of (or even all of) the genomic regions set forth in Table 2 or Table 2A.

In some embodiments the somatic disease state may be clear cell renal cell carcinoma (ccRCC). The at least 10 predetermined genomic regions may be selected from the ccRCC genomic regions set forth in Table 1 or Table 1A. For example, the predetermined genomic regions may comprise at least 10%, 25%, 50%, 75% or at least 90% of (or even all of) the genomic regions set forth in Table 1 or Table 1A.

In a second aspect the present invention provides a computer-implemented method for predicting whether a cell-free DNA (cfDNA)-containing sample obtained from a subject contains DNA from a somatic disease state tissue, the method comprising:
(a) providing methylation sequencing data representing methylation sequencing reads of cfDNA from the sample obtained from the subject, which reads cover at least 10, 25, 50 or at least 100 predetermined genomic regions;
(b) generating metrics from the methylation sequencing data in step (a) that represent one or both of (i) a measure of the degree to which the methylation observed within a sequencing read is similar to a previously determined disease methylation signal; and (ii) a measure of intra-read methylation heterogeneity; and
(c) integrating the metrics generated in step (b) across the at least 10, 25, 50 or at least 100 predetermined genomic regions to obtain a global measure of disease-originating cfDNA in the sample obtained from the subject,
wherein the sample is predicted to contain disease-originating cfDNA if the global measure of disease-originating cfDNA is greater than a background threshold.

In some embodiments the somatic disease state comprises a neoplasm, such as a tumour. The somatic disease state may be a cancer. In particular, the somatic mutation disease may be prostate cancer (PrCa). For example, early stage (localised PrCa), late stage (locally advanced PrCa) or metastatic PrCa. The at least 10 predetermined genomic regions may be selected from the PrCa genomic regions set forth in Table 4 or Table 4A or Table 6 or Table 6A. For example, the predetermined genomic regions may comprise at least 10%, 25%, 50%, 75% or at least 90% of (or even all of) the genomic regions set forth in Table 4 or Table 4A or Table 6 or Table 6A.

In some embodiments the somatic disease state may be oesophageal carcinoma (OAC). The at least 10 predetermined genomic regions may be selected from the OAC genomic regions set forth in Table 3 or Table 3A. For example, the predetermined genomic regions may comprise at least 10%, 25%, 50%, 75% or at least 90% of (or even all of) the genomic regions set forth in Table 3 or Table 3A.

In some embodiments the somatic disease state may be glioblastoma (GBM). The at least 10 predetermined genomic regions may be selected from the GBM genomic regions set forth in Table 2 or Table 2A. For example, the predetermined genomic regions may comprise at least 10%, 25%, 50%, 75% or at least 90% of (or even all of) the genomic regions set forth in Table 2 or Table 2A.

In some embodiments the somatic disease state may be clear cell renal cell carcinoma (ccRCC). The at least 10 predetermined genomic regions may be selected from the ccRCC genomic regions set forth in Table 1 or Table 1A. For example, the predetermined genomic regions may comprise at least 10%, 25%, 50%, 75% or at least 90% of (or even all of) the genomic regions set forth in Table 1 or Table 1A.

In some embodiments each of said at least 10 predetermined genomic regions satisfies at least two (preferably all three) of the following conditions:
(i) the region comprises a differentially methylated region (DMR) comprising a one or more CpGs, wherein said DMR exhibits a significance threshold of q<0.05 or q<0.01 for disease tissue (e.g. cancer tissue) versus healthy control tissue as determined by differential methylation analysis (e.g. MethylKit [Akalin, A., Kormaksson, M., Li, S. et al. methylKit: a comprehensive R package for the analysis of genome-wide DNA methylation profiles. Genome Biol 13, R87 (2012). doi.org/10.1186/gb-2012-13-10-r87])
(ii) the region exhibits a difference in methylation between healthy cfDNA samples and disease tissue (e.g. cancer tissue) within the range 0.6 to 1.0, optionally wherein the difference is determined according to the formula |betaₚₗₐₛₘₐ−round(betaₜᵢₛₛᵤₑ)|>0.6;
(iii) the region comprises at least 2, at least 5 or at least 10 CpGs.

In some embodiments the at least 10 predetermined genomic regions comprises at least 25, at least 50, at least 100, at least 500, at least 1000, at least 5000 or at least 10000 predetermined genomic regions. In certain embodiments, the predetermined regions do not extend to the entire genome or entire set of CpG containing genomic loci of the subject. By selecting informative regions and integrating the disease-originating methylation signal across those regions, embodiments of the present invention were found to strike a balance between sequencing resource and sensitivity for detecting the disease-originating signal through targeted sequencing approaches, but may in future be derived from focused computational analysis of genome-wide data (when the cost of sequencing reduces to the point that offsets the cost of experimental procedures that select/enrich the predetermined regions for targeted sequencing). The predetermined regions may, in some cases, be selected from the genomic regions set forth in any one of Tables 1 to 4 or 6 or Tables 1A to 4A or 6A herein.

In some embodiments, the metric that measures the degree to which the methylation observed within a sequencing read is similar to a previously determined disease methylation signal (e.g. cancer methylation signal), comprises a “read rating” (also known as rare read rating) generated by providing a vector of methylation states for each methylation sequencing read and summing the information content of all methylation states that are concordant with the methylation state for the same region previously determined in a disease tissue sample (e.g. a cancer tissue sample). In some embodiments, the read rating is a penalised read rating, the generation of which further comprises subtracting the information content of all of the methylation states opposite to the methylation state for the same region previously determined in the disease tissue sample (e.g. a cancer tissue sample).

In some embodiments the read rating for each sequencing read is compared with a read rating threshold to determine whether the read is a disease-specific read (e.g. a cancer-specific read), optionally after correcting for background methylation for the same region in samples from healthy controls. The read rating threshold may be a constant value (e.g. zero Shannons or 4.32 Shannons) or a statistical measure of variation from a distribution of read ratings observed for the same region in healthy controls (e.g. the 99^{th} quantile or two or three standard deviations above the mean for healthy controls, or a z-score of 3 or more).

In some embodiments integrating the metrics generated in step (b) across the at least 100 predetermined genomic regions to obtain the global measure comprises summing all the read ratings for all sequencing reads that pass the read rating threshold to be determined to be disease-specific reads (e.g. cancer-specific reads). By integrating or aggregating the read rating metrics in this way, embodiments of the present invention are able to detect disease-originating methylation signals that would not be apparent when focusing on, e.g., a single somatic mutation (c.f. FIGS. 1 and 2).

In some embodiments the metric that measures intra-read methylation heterogeneity, comprises a simple methylation uniformity gauge (SMUGE) score. The SMUGE score may be generated by providing a vector \vec{rₖ} of methylation states for each methylation sequencing read, wherein

$$\vec{{r}_{k}} = [ {r}_{1} , … , {r}_{j} ] , where {r}_{i} ∈ ( - 1 , 1 )$$

where −1 is unmethylated cytosine and 1 is methylated cytosine, j is the number of methylation sites in the predetermined genomic region and wherein the SMUGE score for the k^{th} read is determined according to the formula:

$${smuge}_{k} = {\sum }_{j}^{J - 1} \frac{\vert {r}_{j} - {r}_{j + 1} \vert}{2} .$$

In some embodiments integrating the metrics generated in step (b) across the at least 100 predetermined genomic regions to obtain the global measure comprises summing or averaging the SMUGE scores for all sequencing reads that pass the SMUGE threshold to be determined as disease-specific reads (e.g. cancer-specific reads). By integrating or aggregating the SMUGE metrics in this way, embodiments of the present invention are able to detect disease-originating methylation signals that would not be apparent when focusing on, e.g., a single somatic mutation (c.f. FIGS. 1 and 2b).

In some embodiments, the two metrics are combined and/or utilised together in order to better distinguish disease-originating reads from reads that originate from healthy sources. For example, the metrics that represent (i) the measure of the degree to which the methylation observed within a sequencing read is similar to a previously determined cancer methylation signal; and (ii) the measure of intra-read methylation heterogeneity may be combined into a single score. In some embodiments, the measure of intra-read methylation heterogeneity (e.g. the SMUGE score) is used to classify reads that cannot be unequivocally classified using metric (i) (e.g. read rating) alone. In this embodiment, the method may involve a decision tree approach in which a read is classified as clearly disease, clearly non-disease or uncertain based on the read rating metric. For reads classified as uncertain, the second metric (e.g. SMUGE score) may be used to classify as disease or non-disease originating. Similarly, in some cases, the SMUGE score may be used to classify reads as originating from a disease source of a particular disease stage (e.g. cancer stage) or as originating from a metastatic cancer.

In a third aspect the present invention provides a system comprising:
at least one processor; and
at least one non-transitory computer readable medium containing instructions that, when executed by the at least one processor, cause the at least one processor to perform the method of the second aspect of the invention.

The system may comprise a computing device communicably connected, such as e.g. through a network, to sequence data acquisition means, such as a sequencing machine, and/or to one or more databases storing sequence data. The one or more databases may further store one or more of: training data, learned parameters (e.g. read rating thresholds and/or SMUGE scores obtained from reference datasets of disease sources or healthy controls), clinical and/or sample related information, etc. The computing device may be a smartphone, tablet, personal computer or other computing device. The computing device is configured to implement a method of the second aspect of the invention. In alternative embodiments, a computing device may be configured to communicate with a remote computing device, which is itself configured to implement the method of the second aspect of the invention. In such cases, the remote computing device may also be configured to send the result of the method to the computing device. Communication between the computing device and the remote computing device may be through a wired or wireless connection, and may occur over a local or public network such as e.g. over the public internet. The sequence data acquisition means may be in wired connection with the computing device, or may be able to communicate through a wireless connection, such as e.g. through WiFi and/or over the public internet. The connection between the computing device and the sequence data acquisition means may be direct or indirect (such as e.g. through a remote computer). The sequence data acquisition means is configured to acquire sequence data from a nucleic acid sample. Any sample preparation process that is suitable for use in the determination of a methylation sequencing read may be used within the context of the present invention. The sequence data acquisition means preferably comprises a system for high-throughput, parallel sequencing. In some embodiments, the sequence data acquisition means may comprise a single molecule sequencing platform, such as a PacBio sequencing platform or a Nanopore sequencing platform.

In a fourth aspect the present invention provides a non-transitory computer readable medium or media comprising instructions that, when executed by at least one processor, cause the at least one processor to perform the method of the second aspect of the invention.

In a fifth aspect the present invention provides a method of treatment of a subject suspected of having a cancer, comprising:
performing the method of the first aspect of the invention on a cell free DNA (cfDNA)-containing sample obtained from the subject, wherein the sample is determined to contain circulating tumour DNA (ctDNA) thereby indicating that the subject does have a cancer; and
treatment with curative intent using surgery, radiotherapy, chemotherapy, targeted therapy or immunotherapy; salvage therapy using surgery and/or radiotherapy; administering a minimum therapeutically effective amount of a chemotherapeutic agent, targeted therapy, immunotherapy and/or radiotherapy to the subject. Within this aspect of the invention we measure disease state signal (e.g. tumour-specific signals using rare read rating, SMUGE and integration of the scores, as outlined above) and also assess treatment induced toxicity (e.g. tissue- and cell-specific markers using rare read rating, SMUGE and integration of the scores, as outlined above).

Embodiments of the present invention will now be described by way of example and not limitation with reference to the accompanying figures. However various: further aspects and embodiments of the present invention will be apparent to those skilled in the art in view of the present disclosure.

The present invention includes the combination of the aspects and preferred features described except where such a combination is clearly impermissible or is stated to be expressly avoided. These and further aspects and embodiments of the invention are described in further detail below and with reference to the accompanying examples and figures.

### BRIEF DESCRIPTION OF THE FIGURES

FIG. 1 Schematic illustrating sampling error due to low total copies of ctDNA in early stage cancers (or other low burden somatic disease states). Each line represents cfDNA fragment. Red line represents tumour derived ctDNA. Most blood samples will not contain individual mutant loci.

FIG. 2 Overcoming hard sampling limits using 10³ markers and 10² sequencing reads per locus, improving detection from 1:1000 (single locus sampling limit) to 1:10,000 or 1:100,000.

FIG. 3 Schematic overview of optimised cfDNA methylation workflow (NIMBUS).

FIG. 4 Summary of library yields and fragment lengths resulting from the optimised ssDNA library prep, following either sodium bisulfite (Bisulfite), the standard protocol for enzymatic conversion method (EM-Seq) and optimised enzymatic conversion (NIMBUS). (A) Library concentration yields for the established cfDNA bisulfite conversion protocol (Zymo Lightning) or NIMBUS. (B) NIMBUS generates longer fragments and maintains cfDNA fragmentation pattern, consistent with non-destructive DNA methylation conversion. (C) cfDNA recovery in low-input samples using the standard protocol for EM-Seq and NIMBUS protocol. NIMBUS recovers more genomic sequence from the same input. (D) Information content in low-input samples using the standard protocol for EM-Seq and NIMBUS. NIMBUS protocol recovers more information in form of the total number of CpGs. This improves the dynamic range by increasing the information content of each read and total information content when comparing with EM-Seq.

FIG. 5 Biotin-NIMBUS overview. (A) This modified NIMBUS protocol includes the addition of biotinylated sequencing adapters before the enzymatic conversion step, allowing bead coupling throughout the conversion process, in order to minimise sample loss during the multiple conversion steps (‘clean-up’). (B) Pilot data showing that the biotin-NIMBUS protocol can improve recovery of informative reads when compared with the distribution of informative reads from the standard NIMBUS protocol.

FIG. 6 DNA methylation signal from an example locus, showing the difference between average methylation signals and read rating scores used in NIMBUS that are used to dichotomise tumour derived signals and facilitate sensitive ctDNA detection. (A) Example loci showing average DNA methylation signal at single base resolution in prostate tissue samples (from EPICseq, bisulfite capture sequencing analysis). Each boxplot represents the average methylation signal for one CpG from this example locus in tumour-benign paired tissue samples from 188 men with prostate cancer (blue represents signal in benign and red represents signal in tumour samples). (B) Read Rating score summary of the representative marker locus, generated using NIMBUS analysis of a cfDNA blood plasma sample from a metastatic patient. Each horizontal line represents a single sequencing read and each square box on those lines represents a single CpG, coloured red when methylated and blue if unmethylated. The Read Rating score allows for clear separation of signal coming from tumour tissue (high Read Rating score) and non-tumour tissues (low Read Rating score) (this simplified summary diagram represents data from a random subset of 30 reads from 1 locus in 1 patient).

FIG. 7 NIMBUS detection accuracy (Area Under ROC Curve) for (A) early (localised), (B) late stage (locally advanced) and (C) metastatic prostate cancer (sensitivity and specificity assessment using age-matched cancer-free controls from men with clinical indication for diagnostic biopsy, including suspicious lesions on MRI scans and/or significantly elevated PSA). Pilot study results from n=72 samples.

FIG. 8 Summary of SMUGE scores in prostate cancer cfDNA in age-matched disease-free controls, early stage (localised PrCa), late stage (locally advanced PrCa) and metastatic prostate cancer plasma cfDNA samples (n=72 samples). Combined with Read Rating score, SMUGE scores can be used to improve the detection of ctDNA in blood plasma samples from (C) early-stage, (D) late-stage and (E) metastatic prostate cancer (using a decision tree model where SMUGE allows further discrimination of tumour-derived reads that are classified as ‘uncertain’ based on read rating alone).

FIG. 9 Esophageal adenocarcinoma (EAC) NIMBUS scores (A) for late stage (locally advanced) esophageal adenocarcinoma and control samples. NIMBUS detection accuracy (Area Under ROC Curve) for (B) late stage (locally advanced) esophageal adenocarcinoma (sensitivity and specificity assessment using age-matched cancer-free controls). Pilot study results from n=35 samples.

FIG. 10 Clear cell renal cell carcinoma NIMBUS scores (A) for late stage (locally advanced) clear cell renal cell carcinoma and control samples. NIMBUS detection accuracy (Area Under ROC Curve) for (B) late stage (locally advanced) clear cell renal cell carcinoma (sensitivity and specificity assessment using age-matched cancer-free controls from). Pilot study results from n=21 samples.

FIG. 11 Clear cell renal cell carcinoma-treatment response prediction SMUGE scores (A) for late stage (locally advanced) clear cell renal cell carcinoma for treatment responders (n=5) and non-responders (n=5). SMUGE treatment prediction accuracy (Area Under ROC Curve) for (B) late stage (locally advanced) clear cell renal cell carcinoma. Pilot study results from n=10 samples.

FIG. 12 Clear cell renal cell carcinoma: treatment response monitoring NIMBUS scores for late stage (locally advanced) clear cell renal cell carcinoma undergoing surgical treatment. NIMBUS signal is normalized to pre-treatment score. Pilot study results from n=10 samples.

FIG. 13 Clear cell renal cell carcinoma: treatment response monitoring NIMBUS scores for late stage (locally advanced) clear cell renal cell carcinoma undergoing tyrosine kinase inhibitors (TKI) treatment (see Table 5). NIMBUS signal is normalized to pre-treatment score. Sample as grouped as responding to treatment (gold-right-hand box plot) and not responding to treatment (grey-left-hand box plot). Pilot study results from n=10 samples.

FIG. 14 Prostate cancer detection: SMUGE. SMUGE score (A) for early (localised), late stage (locally advanced) and metastatic prostate cancer, Kruskal-Wallis p-value=2.26e-06. SMUGE detection accuracy (Area Under ROC Curve) for (B) all stages of prostate cancer (sensitivity and specificity assessment using age-matched cancer-free controls from men with clinical indication for diagnostic biopsy, including suspicious lesions on MRI scans and/or significantly elevated PSA). Pilot study results from n=72 samples.

FIG. 15 Esophageal adenocarcinoma detection: SMUGE. SMUGE scores (A) for late stage (locally advanced) esophageal adenocarcinoma and control samples. SMUGE detection accuracy (Area Under ROC Curve) for (B) late stage (locally advanced) esophageal adenocarcinoma (sensitivity and specificity assessment using age-matched cancer-free controls from). Pilot study results from n=35 samples.

FIG. 16 Clear cell renal cell carcinoma detection: SMUGE. SMUGE scores (A) for late stage (locally advanced) clear cell renal cell carcinoma and control samples. NIMBUS detection accuracy (Area Under ROC Curve) for (B) late stage (locally advanced) clear cell renal cell carcinoma (sensitivity and specificity assessment using age-matched cancer-free controls from). Pilot study results from n=21 samples.

FIG. 17 Prostate cancer: risk stratification. Principal components of regions with high SMUGE score (A) for early (localised, low-risk, pink) and late stage (locally advanced, high-risk, orange) prostate cancer (n=40). (B) SMUGE based detection of high-risk prostate cancer (Area Under ROC Curve) using leave-one-out cross-validation with elastic-net regression.

FIG. 18 NIMBUS: prostate cancer survival prediction. Prostate cancer survival for localized prostate cancer with high NIMBUS score (violet—lower survival curve) and low NIMBUS score (pink—higher survival curve), hazard ratio=56.3. Prostate cancer patients were followed up for average of 4.6 years (2.4-6 years), n=40.

FIG. 19 Biotinylated NIMBUS method performance in prostate cancer. Prostate cancer plasma samples prepared with standard protocol (n=20), normal plasma cancer samples prepared with standard protocol (n=37) and prostate cancer samples prepared with biotinylated primers (n=2) were down-sampled ten times to random subset of 100 genomic regions and total of 1000 reads. Prediction accuracy (Area Under ROC Curve) curves for default protocol (black) and biotinylated protocol (gold) (A). Distribution of AUC for default and biotinylated protocols (B). Biotinylated samples demonstrated superior performance when compared to standard protocol.

FIG. 20 Prostate cancer: treatment response monitoring. NIMBUS scores for late-stage prostate cancer undergoing surgical treatment. NIMBUS signal is normalized to pre-treatment score. Pilot study results from n=22 samples.

FIG. 21 Glioma detection: NIMBUS. NIMBUS scores (A) for glioma and control samples. NIMBUS detection accuracy (Area Under ROC Curve) for (B) glioma (sensitivity and specificity assessment using age-matched cancer-free controls from). Pilot study results from n=34 samples.

FIG. 22 Glioma: risk stratification: NIMBUS and SMUGE combination. SMUGE scores for reads with high Rare Read Rating (A) for low-grade glioma and high-grade glioma. Combined Rare Read Rating and SMUGE risk stratification accuracy (Area Under ROC Curve) for (B) low-grade glioma and high-grade glioma differentiation. Pilot study results from n=24 samples.

FIG. 23 Glioma detection: SMUGE. SMUGE scores (A) for glioma and control samples. SMUGE detection accuracy (Area Under ROC Curve) for (B) glioma (sensitivity and specificity assessment using age-matched cancer-free controls from). Pilot study results from n=34 samples.

### DETAILED DESCRIPTION

In describing the present invention, the following terms will be employed, and are intended to be defined as indicated below.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

A “sample” as used herein may be a biological fluid, a cell or tissue sample (e.g. a biopsy), or an extract from which genomic material can be obtained for methylation sequencing analysis. In particular, the sample may be a tumour sample. The sample may be one which has been freshly obtained from a subject or may be one which has been processed and/or stored prior to making a determination (e.g. frozen, fixed or subjected to one or more purification, enrichment or extractions steps). In particular, the sample may be a cell or tissue culture sample that has been derived from a tumour. As such, a sample as described herein may refer to any type of sample comprising cells or genomic material derived therefrom, whether from a biological sample obtained from a subject, or from a sample obtained from e.g. a cell line. The sample is preferably from a mammalian (such as e.g. a mammalian cell sample or a sample from a mammalian subject, including in particular a model animal such as mouse, rat, etc.), preferably from a human (such as e.g. a human cell sample or a sample from a human subject). Further, the sample may be transported and/or stored, and collection may take place at a location remote from the genomic sequence data acquisition (e.g. sequencing) location, and/or the computer-implemented method steps may take place at a location remote from the sample collection location and/or remote from the genomic data acquisition (e.g. sequencing) location (e.g. the computer-implemented method steps may be performed by means of a networked computer, such as by means of a “cloud” provider). A “tumour sample” refers to a sample that contains tumour cells or genetic material derived therefrom. The tumour sample may be a cell or tissue sample (e.g. a biopsy) obtained directly from a tumour.

A “cell free DNA (cfDNA)-containing sample” or “liquid biopsy” as used herein is a sample obtained from the subject and which includes one or more DNA molecules, typically fragmented, that are not contained within intact cells (though the sample may contain one or more cells either intentionally or as trace impurities). The cfDNA-containing sample may be a tissue sample (e.g. FNA), proximal sample (e.g. urine, saliva) or a minimally invasive distal sample (e.g. plasma or other blood sample).

As used herein “somatic disease state” refers to any disease associated with a change in the DNA sequence or epigenetic change (e.g. methylation change) in one or more somatic cells of a multicellular organism. Somatic disease state as used herein expressly includes any and all cancers whether thought to be driven by somatic mutations or not, and specifically includes germ cell cancers. The cancer may be ovarian cancer, breast cancer, endometrial cancer (uterus/womb cancer), kidney cancer (renal cell), lung cancer (small cell, non-small cell and mesothelioma), brain cancer (gliomas, astrocytomas, glioblastomas (GBM)), melanoma, merkel cell carcinoma, clear cell renal cell carcinoma (ccRCC), lymphoma, gastrointestinal cancer (e.g. colorectal cancer), small bowel cancers (duodenal and jejunal), leukemia, pancreatic cancer, hepatobiliary tumours, liver cancer (e.g. hepatocellular carcinoma), germ cell cancers, prostate cancer, head and neck cancers, bladder cancer, thyroid cancer, oesophagal cancer (OAC), melanoma (e.g. uveal melanoma), cutaneous squamous cell carcinoma and sarcomas. For example, the cancer may be head and neck squamous cell carcinoma (HNSCC), hepatocellular carcinoma (HCC), colorectal cancer (CRC), different types of lung cancer (LC), clear cell renal cell carcinoma (ccRCC), prostate cancer (PrCa), breast cancer (BC), bladder urothelial carcinoma (BUC), esophageal squamous-cell carcinoma (ESCC), uveal melanoma (UV) and cutaneous squamous cell carcinoma (csCC). Other somatic disease states that are characterised by over-represented epigenetic signatures in cfDNA from affected individuals contemplated herein include neurodegenerative diseases, inflammatory diseases, tissue trauma, liver disease, cardiovascular disease, developmental disorders and aging related diseases. For example, the somatic disease state may be multiple sclerosis, schizophrenia, Parkinson's, Fragile X-associated tremor/ataxia syndrome, dementia, Alzheimer's, diabetes, arthritis, non-alcoholic fatty liver disease, cirrhosis, coronary artery disease, atrial fibrillation, chronic obstructive pulmonary disease, lupus, cerebral palsy or other tissue specific developmental disorders. In some cases the somatic disease state may be a pre-cancerous disease state such as Barrett's oesophagus.

As used herein “subject” refers to an animal or human. The subject may be mammalian (e.g. a human, a non-human primate, a cat, dog, horse, donkey, sheep, pig, goat, cow, mouse, rat, rabbit or guinea pig). The subject is preferably from a human (such as e.g. a human at risk of developing a somatic mutation disease such as a malignant neoplasm).

As used herein “treatment” and “therapy” refer to reducing, alleviating or eliminating one or more symptoms of the disease which is being treated, relative to the symptoms prior to treatment.

The systems and methods described herein may be implemented in a computer system, in addition to the structural components and user interactions described. As used herein, the term “computer system” includes the hardware, software and data storage devices for embodying a system or carrying out a method according to the above-described embodiments. For example, a computer system may comprise a processing processing unit such as a central (CPU) and/or graphics processing unit (GPU), input means, output means and data storage, which may be embodied as one or more connected computing devices. Preferably the computer system has a display or comprises a computing device that has a display to provide a visual output display. The data storage may comprise RAM, disk drives or other computer readable media. The computer system may include a plurality of computing devices connected by a network and able to communicate with each other over that network. It is explicitly envisaged that computer system may consist of or comprise a cloud computer.

The methods described herein may be provided as computer programs or as computer program products or computer readable media carrying a computer program which is arranged, when run on a computer, to perform the method(s) described herein. As used herein, the term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

The following is presented by way of example and is not to be construed as a limitation to the scope of the claims.

### EXAMPLES

The present inventors have developed a novel pipeline (NIMBUS: Non-destructive integration of methylation to boost underlying signals) for detecting cancer in blood plasma samples by detection of cancer-specific methylation patterns detectable in circulating tumour DNA. NIMBUS (also referred to herein as “the pipeline”) overcomes hard sampling by allowing simultaneous detection of broad and highly recurrent epigenomic changes that occur in many cancer types.

NIMBUS maximises the number of informative molecules for DNA methylation analysis and can be applied to low-input DNA samples (e.g. single cell, small biopsies, proximal samples, low purity or low cellularity samples) from a range of samples types including tissue samples (e.g. biopsy), proximal samples (e.g. urine, saliva, FNA) or minimally invasive distal samples (e.g. plasma or other blood sample).

### NIMBUS Approach:

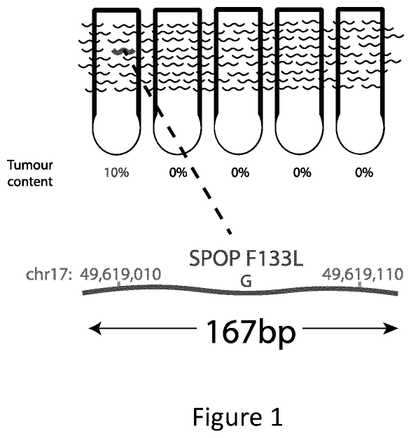

The low number of copies of each genomic locus results in a hard limit for ctDNA detection in early-stage cancer. However, each ctDNA fragment (~167 bp) represents an entire genome copy that is also sampled in a given blood draw (i.e ~1.8×10⁷ ctDNA fragments of 167 bp, per copy of the human genome) (FIG. 1). This highlights an opportunity to increase the sensitivity of ctDNA detection by undertaking a broader analysis of tumour-specific features that can be measured in ctDNA. Such tumour-specific features are methylation markers.

### Outliner of Pipeline Steps:

Defining 10²-10⁴ tumour-specific DNA methylation markers that can be analysed in parallel (panel analysis) to improve ctDNA detection in low burden samples. The markers were selected comparing tissue biopsies from healthy and tumour tissues by applying statistical analysis.
Recovering larger numbers of intact ctDNA molecules with a novel wet-lab workflow for ctDNA methylation analysis, using an optimised and unique workflow;
Applying novel analysis workflow integrating the tumour specific signal along the read (score 1: read rating (“RR”)) as well as heterogeneity of the methylation pattern (score 2: “SMUGE”).
Creating a global measure of ctDNA levels by integrating signals across 10²-10⁴ tumour-specific markers.

### Example 1-Optimised Conversion and Methylation Sequencing Workflow Reagents

### 1. Methyl-Cytosine Conversion

| Material | Cat No | Vendor |
|---|---|---|
| EM-seq Conversion | NEB E7125S or L | New England Biolabs |
| Module | | |
| Agencourt AMPure XP | A63880 | Beckman Coulter |
| 100% Ethanol | | |
| Nuclease-free Water | AM9930 | Ambion |

### 2. Library Preparation

| Material | Cat No | Vendor |
|---|---|---|
| Accel-NGS Methyl-Seq | 30096 | Swfit BioSciences |
| DNA Library Kit | | |
| Methyl-Seq Unique | 39096 | Swfit BioSciences |
| Dual Indexing Kit | | |
| (24 indices, 96 rxns) | | |
| 100% Ethanol | | |
| Agencourt AMPure XP | A63880 | Beckman Coulter |
| KAPA HiFi Uracil+ Kit | 07959052001 | Roche |
| High Sensitivity | | Agilent |
| D1000/D5000 Reagents | | |
| High Sensitivity | | Agilent |
| D1000/D5000 | | |
| ScreenTape | | |
| Qubit dsDNA HS Assay | Q32850 | Life Technologies |
| Kit | | |
| Qubit Assay tubes | Q32856 | Life Technologies |

### 3. Nimblegen Target Enrichment Kit (Option 1)

| Material | Cat No | Vendor |
|---|---|---|
| SeqCap Epi Choice S 48 | 07138946001 | Roche |
| Rxn | | |
| SEQCAP EPI ACCESSORY | 07145519001 | |
| KIT, (24 rxn) | | |
| SEQCAP PURE CAPTURE | 06977952001 | |
| BEAD KIT | | |
| SEQCAP HYB AND WASH | 05634261001 | |
| KIT, (24 rxn) | | |
| HYPERCAP UNIVERSAL | 08286396001 | |
| BLOCKING OLIGO (24 | | |
| rxn) | | |

### 4. Twist Targeted Methylation Sequencing Kit (Option 2)

| Material | Cat No | Vendor |
|---|---|---|
| Custom Methylation | Contact Twist | Twist Biosciences |
| Panel | Biosciences | |
| Twist Universal | Depends on no. of rxn | Twist Biosciences |
| Blockers | | |
| Twist Methylation | Depends on no. of rxn | Twist Biosciences |
| Enhancer | | |
| Twist Binding and | Depends on no. of rxn | Twist Biosciences |
| Purification Beads | | |
| Twist Fast | Depends on no. of rxn | Twist Biosciences |
| Hybridization and | | |
| Wash Kit (2 Boxes) | | |
| Twist Fast | | |
| Hybridization Reagent | | |
| (Box1 of 2) | | |
| Twist Fast Wash | | |
| Buffers | | |
| (Box 2 of 2) | | |
| KAPA HotStart Ready | 7958927001 | Roche |
| Mix | Depends on no. of rxn | |

Optimised protocol for cfDNA using EM-seq Conversion Module Starting Material: 1 ng-200 ng double stranded cfDNA. The protocol may optimally use around 10 ng of DNA as measured by Qubit. Process samples in 0.2 mL PCR DNase-RNAse tubes.

Recommended to use low-bind pipette tips throughout to prevent loss of material.

### 1. DNA PREPARATION

1.1. cfDNA

A range of starting input cfDNA should be in a 28 μl final volume. If necessary, dilute in water. There should be no/little EDTA present in the sample to avoid interference with downstream conversion.

### 1.2. Genomic DNA or any Double Stranded DNA

A range of starting input dsDNA should be fragmented using Covaris. The optimised shearing size range is 200-300 bp. Please follow the manufacturer instruction provided from Covaris webpage.

### 2. Oxidation of 5-Methylcytosines and 5-Hydroxymethylcytosines

2.1. Prepare TET2 Buffer. Use option A if you have E7125S/E7125L (24 r×n) and option B if you have E7125L (96 r×n).

Note: The TET2 Reaction Buffer Supplement is a powder. Centrifuge before use to ensure it is at the bottom of the tube.

### 2.1A.

24 Reaction Kit: Add 100 μl of TET2 Reaction Buffer to one tube of TET2 Reaction Buffer Supplement and mix well.

### 2.1B.

96 Reaction Kit: Add 400 μl of TET2 Reaction Buffer to one tube of TET2 Reaction Buffer Supplement and mix well.

NOTE: The reconstituted buffer should be stored at −20° C. and discarded after 4 months.

2.2. Dilute the 500 mM Fe(II) Solution (yellow) by adding 1 μl to 1249 μl of water.

NOTE: Use the solution immediately, do not store it. Discard after use.

2.3. On ice, add the following components directly to the 28 μl DNA (from Step 1.1).

| COMPONENT | VOLUME |
|---|---|
| TET2 Reaction Buffer (reconstituted) | 10 μl |
| Oxidation Supplement | 1 μl |
| Oxidation Enhancer | 1 μl |
| TET2 | 4 μl |
| DTT | 1 μl |

Mix thoroughly by vortexing, centrifuge briefly, then add

| COMPONENT | VOLUME |
|---|---|
| Diluted Fe (II) Solution (from Step 2.2) | 5 μl |
| Total Volume | 50 μl |

For multiple reactions, a master mix of the reaction components can be prepared before addition to the sample DNA. 5mC/5hmC oxidation is initiated by the addition of the Fe(II) solution to the reaction after the addition of master mix.

Mix thoroughly by vortexing or by pipetting up and down at least 10 times, centrifuge briefly.

2.4. Incubate at 37° C. for exactly 1 hour in a thermocycler with the heated lid set to ≥45° C.

2.5.1 Transfer the samples to ice for 2 minutes

2.5.2 Pulse Spin and replace on ice

2.5.3 Immediately add 1 μl of Stop Reagent (yellow).

| COMPONENT | VOLUME |
|---|---|
| (yellow) Stop Reagent | 1 μl |
| Total Volume | 51 μl |

Mix thoroughly by vortexing or by pipetting up and down at least 10 times and centrifuge briefly.

2.6. Incubate at 37° C. for 30 minutes, (with heated lid set to ≥45° C. or on) then at 4° C. hold in a thermocycler.

Safe Stopping Point: Samples can be stored overnight at either 4° C. in the thermocycler or at −20° C. in the freezer.

### 3. Clean-Up of TET2 Converted DNA

Retrieve AMpure beads from fridge and bring to room temperature (30 min)

Retrieve NEB elution buffer from the −20 freezer to thaw room temperature

3.1. Vortex AMpure beads to resuspend well.

3.2. Add resuspended AMPure XP to each sample. Mix well by pipetting up and down at least 10 times. Be careful to expel all of the liquid out of the tip during the last mix.

3.3. Incubate samples on bench top at room temperature.

3.4. Place the tubes against an appropriate magnetic stand to separate the beads from the supernatant.

3.5. Carefully remove and discard the supernatant.

Be careful not to disturb the beads that contain DNA targets (Caution: do not discard the beads).

3.6. Add 200 μl of 80% freshly prepared ethanol to the tubes while in the magnetic stand.

Be careful not to disturb the beads that contain DNA targets when discarding the supernatant.

3.7. Repeat the wash once for a total of two washes.

Pulse spin and put tubes back on to magnetic and remove residual ethanol using a p10 pipette tip.

3.8. Air dry the beads with tubes open for up to 2 minutes, whilst tubes are on the magnet.

3.9. Remove the tubes from the magnetic stand. Elute the DNA target from the beads by adding 17 μl of Elution Buffer (white), with single channel pipette.

3.10. Mix well by pipetting up and down 10 times. Incubate for at 5 minutes at room temperature.

3.11. Place the tube on the magnetic stand. After 3 minutes (or whenever the solution is clear), transfer 16 μl of the supernatant to a new PCR tube.

Safe Stopping Point: Samples can be stored overnight at −20° C.

4. Denaturation of cfDNA

The DNA can be denatured using 0.1 N Sodium Hydroxide. (stored at room temperature) (Check pH of solution)

### Sodium Hydroxide

4.1. Retrieve 0.1 N NaOH.

4.2. Pre-heat thermocycler to 50° C., with the heated lid set to ≥60° C. or on.

4.3. Add 4 μl 0.1 N NaOH to the 16 μl of oxidized DNA. Vortex to mix or by pipetting up and down at least 10 times, centrifuge briefly.

4.4. Incubate at 50° C. for 10 minutes in the pre-heated thermocycler, with the heated lid set to ≥60° C. or on.

4.5. Immediately place on ice.

4.6. Proceed immediately to Section 5.

### 5. Deamination of Cytosines

5.1. On ice, add the following components to the 20 μl of denatured DNA.

| COMPONENT | VOLUME |
|---|---|
| Nuclease-free water | 68 μl |
| (orange) APOBEC Reaction Buffer | 10 μl |
| (orange) BSA | 1 μl |
| (orange) APOBEC | 1 μl |
| Total volume | 100 μl |

5.2. For multiple reactions, a master mix of the reaction components can be prepared before addition to the denatured DNA.

5.3. Mix thoroughly by vortexing or by pipetting up and down at least 10 times, centrifuge briefly. Incubate at 37° C. for 3 hours then at 4° C. in a thermocycler, with the heated lid set to ≥45° C. or on.

Safe Stopping Point: Samples can be stored overnight at either 4° C. in the thermocycler or at −20° C. in the freezer.

### 6. Clean-Up of Deaminated DNA

6.1. Vortex Sample Purification Beads to resuspend.

6.2. Add resuspended AMPure XP Beads to each sample. Mix well by pipetting up and down at least 10 times. Be careful to expel all of the liquid out of the tip during the last mix.

6.3. Incubate samples on bench top at room temperature.

6.4. Place the tubes against an appropriate magnetic stand to separate the beads from the supernatant.

6.5. Carefully remove and discard the supernatant. Be careful not to disturb the beads that contain DNA targets (Caution: do not discard the beads).

6.6. Add 200 μl of 80% freshly prepared ethanol to the tubes while in the magnetic stand. Carefully remove and discard the supernatant, be careful not to disturb the beads that contain DNA targets.

6.7. Repeat the wash once for a total of two washes. Be sure to remove all visible liquid after the second wash using a p10 pipette tip. Air dry the beads.

6.8. Remove the tubes from the magnetic stand. Elute the DNA target from the beads by adding 16 μl of Low TE buffer provided from Swift Biosciences.

6.9. Mix well by pipetting up and down 10 times. Incubate for 2 minutes at room temperature.

If necessary, quickly spin the sample to collect the liquid from the sides of the tube before placing back on the magnetic stand. 6.10. Place the tube on the magnetic stand.

After 3 minutes (or whenever the solution is clear), transfer total supernatant (~16 ul, but avoid excessive bead carryover) to a new PCR tube.

Safe Stopping Point: Samples can be stored overnight at −20° C.

### Protocol for Converted-Ready Library

### Denaturation

1. Due to the short incubation time of the Denaturation step, pre-assemble all of the reagents of the Adaptase Reaction Mix (see next step for recipe), and place on ice.
2. Pre-heat the thermocycler to 95° C.
3. Place the samples in the thermocycler, programmed at 95° C. for 2 minutes with lid heating ON (using first ‘HOLD/Infinite’ step of ‘PCR program’ listed below).
4. Upon completion, place tube(s) on ice immediately for at least for 2 minutes. Proceed directly to the Adaptase step to preserve the maximum amount of ssDNA substrate.

### PCR Program Setting:

Lid heat—ON
95° C.—Infinite mode Denaturation Ready
95° C.—2 minutes. Denaturation
37° C.—Infinite mode Adaptase Ready
37° C.—15 minutes. Adaptase Step 1.
95° C.—2 minutes Adaptase Step 2.
4° C.—Hold/Infinite

### Adaptase

6. Load the Adaptase Thermocycler Program on the thermocycler and pause it at the first step to pre-heat to 37° C. until all samples are loaded.

| Reagents | Volume per sample |
|---|---|
| LOW EDTA TE | 11.5 μl |
| Buffer G1 | 4.0 μl |
| Reagent G2 | 4.0 μl |
| Reagent G3 | 2.5 μl |
| Enzyme G4 | 1.0 μl |
| Enzyme G5 | 1.0 μl |
| Enzyme G6 | 1.0 μl |
| Total Volume | 25.0 μl |

7. Add 25 μl of the pre-assembled Adaptase Reaction Mix to each PCR tube containing a 15 μl DNA sample and mix by pipetting or gentle vortexing until homogeneous. Spin down.
8. Place the samples in the thermocycler and run the program, with lid heating ON. Proceed immediately to next step.

Thermocycler Program
37° C., 15 min
95° C., 2 min
4° C., hold

### Extension

9. Load the Extension Thermocycler Program on the thermocycler and pause it at the first step to pre-heat to 98° C. until all samples are loaded.
10. Add 44 μl of the pre-mixed Extension Reaction Mix (listed in the table below) to each PCR tube containing 40 μl of the Adaptase Reaction, using reagents in the order listed.

| Reagents | Volume Per Sample |
|---|---|
| Reagent Y1 | 2 μl |
| Reagent Y2 | 42 μl |
| Total Volume | 44 μl |

11. Mix by pipetting or gentle vortexing until homogenous. Spin down.
12. Place the samples in the thermocycler and run the program, with lid heating ON.

Thermocycler Program
98° C., 1 min
62° C., 2 min
65° C., 5 min
4° C., hold

### Post-Extension CLEAN-UP

Prepare a fresh solution of 80% EtOH.

Ensure the magnetic beads are at room temperature and vortex beads to homogenize the suspension before use.
1. Add beads to each sample. Mix by vortexing. Quick spin the samples in a tabletop microcentrifuge.
2. Incubate the samples at room temperature (off the magnet).
3. Place the sample on a magnetic rack until the solution clears and a pellet is formed (~2 minutes).
4. Remove and discard the supernatant without disturbing the pellet (less than 5 μl may be left behind). Leave the tubes on the magnet.
5. Add 200 μl of freshly prepared 80% ethanol solution to the sample while it is still on the magnetic rack. Use care not to disturb the pellet. Immediately and carefully remove the ethanol solution.
6. Repeat step 7 once more for a second wash with the 80% ethanol solution.
7. Quick spin the samples in a tabletop microcentrifuge and place back on the magnetic rack. Remove any residual ethanol solution from the bottom of the tube.
8. Add 15 μL Elution Volume of Low EDTA TE buffer and re-suspend the pellet. Mix well by pipetting up and down until homogenous. Incubate at room temperature for 2 minutes off the magnet, then place the tube on the magnet.
9. Transfer entire eluate to a new 0.2 mL PCR tube. Ensure that eluate does not contain magnetic beads (indicated by brown coloration in the eluate). If magnetic beads are present, pipette eluate into a new tube, place on magnet, and transfer eluate again.

### Ligation

13. Add 15 μl of the pre-mixed Ligation Reaction Mix (listed in the table below) to a new PCR tube containing 15 μl of the Post-Extension eluate. For the Ligation Reaction Mix, use reagents in the order listed below and note Enzyme B3 should be added to the master mix just before use.

| Reagents | Volume Per Sample |
|---|---|
| Buffer B1 | 3 μl |
| Reagent B2 | 10 μl |
| Enzyme B3 | 2 μl |
| Total Volume | 15 μl |

14. Mix by pipetting or gentle vortexing until homogenous. Spin down.
15. Place the samples in the thermocycler programmed at 25° C. for 15 minutes with lid heating ON, followed by a 4° C. hold
16. Bead clean up. Follow the instruction above but with elution volume of 20 μL.

| Sample Volume | Bead volume | Elution Volume |
|---|---|---|
| 30 ul | 36 μl (ratio: 1.2) | 20 μl |

### Indexing PCR

17. Add 5 μl of the appropriate indexed adapter primer(s) directly to each sample.
18. Add 25 μl of the already pre-mixed Indexing PCR Reaction Mix to each PCR tube containing 25 μl of sample.
19. Uracil tolerant enzyme should be added to the master mix just before use.
20. Bead Clean up. Follow the instruction above but with elution volume of 20 μL.
21. Library quantification can be followed using Tapestation or Bioanalyzer to determine the size distribution and quantity of libraries.

Sequence capture utilised the Nimblegen Targeted Enrichment kit or alternatively, the Twist Biosciences Twist Targeted Methylation Sequencing Kit (according to the manufacturers protocol).

### Example 2—ctDNA Detection Using NIMBUS

2.1 ctDNA Methylation Markers Identification

Tumour-specific ctDNA methylation markers were identified in tumour tissue analysis, followed by marker triage in cfDNA plasma samples by comparing cases (cancer patients) and healthy controls (age-matched individuals with clinical indications of cancer, but confirmed as disease-free on biopsy). We then performed our analysis on the healthy control cfDNA samples to further narrow the markers to the ones that are likely to show large separation between controls and tumour derived ctDNA. We have identified ~10⁴ tumour-specific markers in blood plasma cfDNA. The genomic locations of the tumour-specific markers are set forth in Tables 1~4 or 6 or Tables 1A-4A or 6A.

Table 1/1A—clear cell renal cell carcinoma (ccRCC). The first entry corresponds to the chromosome number in which the region is found. This is followed by a colon and then a numerical range. The first number is the start position (i.e. genomic coordinate) and the following number is the end position (i.e. genomic coordinate. The position numbering is in accordance with the reference human genome version [GRCh38]. For example, the first region “1:605331-605509” means chromosome 1, the region between position 605331 and 605509. The next region is shown after the semicolon.

Table 2/2A—glioblastoma (GBM). The numbering convention for the regions is as for Table 1.

Table 3/3A—oesophageal carcinoma (OAC). The numbering convention for the regions is as for Table 1.

Table 4/4A—prostate cancer (PrCa). The numbering convention for the regions is as for Table 1.

Table 6/6A—prostate cancer (PrCa) from CaNCaP Study. The numbering convention for the regions is as for Table 1.

Without wishing to be bound by any particular theory, the present inventors believe that further markers suitable for use in the NIMBUS method of the present invention may be identified in the genome by extending the marker discovery methods described herein, e.g. to other cancers and/or other patient groups. In particular, the present inventors aim to provide a broad definition that encompasses: 1/ the original test cases (cancer detection, MRD detection, treatment response monitoring); and 2/ future applications in other disease spaces (e.g. pre-neoplastic conditions and non-cancer diseases such as neurological/brain pathologies including diseases that currently have functional diagnoses (rather than definitive diagnostic/biomarker tests). In this process we define a large panel of markers (~10³-10⁴ loci) that are informative across a broad cohort of affected individuals with a given disease/pathology and therefore prospective analysis for the detection, allow high-sensitivity diagnosis and/or monitoring of disease states (this is in contrast to other leading methods for cancer/disease detection that aim at: 1/ multi-cancer detection but have limited sensitivity for early stage cancer [e.g. Galleri]; 2/ orthogonal high-sensitivity assays require either patient-specific mutation panel design [e.g. INVAR, RaDaR, TARDIS, AMP-VariantPlex]; or 3/ large gene panel assays that are suitable only for cancer types with high mutation burdens [e.g. CAPP-SEQ/AVENIO, TAm-Seq/InVision]).

In our differential methylation analysis of disease versus control samples (e.g. tumour tissue vs pathologically normal tissue) we commonly identify >=10³-10⁵ significantly differentially methylated marker loci in each disease setting (with a threshold of q<0.01 using established methods for differential methylation analysis [e.g. MethylKit]), providing a large search space for disease-specific markers that could be included in disease detection assays. The core concept of our marker selection workflow is to prioritise a large panel of disease-specific markers (in the order of 10³-10⁴ markers) to overcome sampling limits for rare tumour/disease signal detection in limited volume blood samples. We filter and rank these markers to define a core set of the most likely informative markers in minimally invasive samples (using disease-free ‘healthy control’ cell-free DNA methylome datasets to remove confounded markers and also leverage the number of informative CpGs at each marker site to enrich for ‘high information content’ markers), aiming for a final marker panel in the order of ~10³-10⁴ disease-specific methylation marker loci. This approach has worked successfully in 4 cancer types tested to date (prostate, renal, glioma and oesophageal cancer).

### Functional Definition of Markers

1. Identify disease-specific differentially methylated markers (differentially methylated cytosines (DMCs) and differentially methylated regions (DMRs)) with a significance threshold of q<0.01-0.05 for disease vs control tissue.
2. Filter disease-specific markers (optional, in silico filtering) against healthy control (disease-free) cfDNA datasets to remove markers with confounded signal in non-diseased populations (e.g. markers which show similar patterns of methylation in blood cells or other cell types commonly represented in non-diseased cfDNA samples). This criteria is: range of 0.6-1.0 difference in methylation (between healthy CfDNA and expected value from diseased tissue).

$$\vert {beta}_{plasma} - round ( {beta}_{tissue} ) \vert > 0.6$$

$${beta}_{plasma} - mean CpG methylation in plasma$$

$${beta}_{tumour} - mean CpG methylation in diseased tissue$$

3. Filter disease-specific markers by CpG content to prioritise markers with greater specificity: minimum of 2 CpGs per marker locus (more CpGs per marker locus allows greater specificity in distinguishing disease-specific signals from background technical/biological noise).
4. Experimentally test t panel of disease-specific markers using a disease-free cohort (e.g. cfDNA samples from men in the diagnostic pathway with suspected prostate cancer who are cancer-free on biopsy). The results of this initial test are used to further filter potentially confounded markers as defined by 99^{th} quantile of the read rating in the given DMR.
   2.2 ctDNA Recovery Yield Improvement

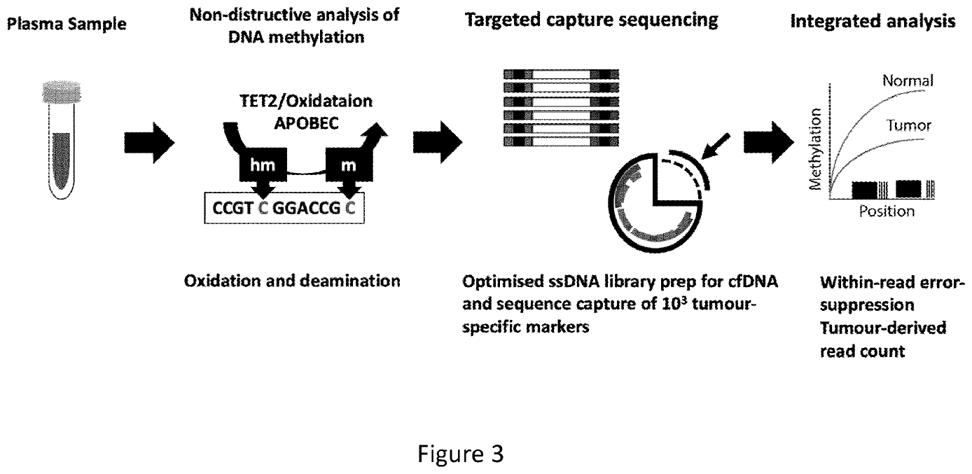

The present inventors have established new wet lab methods to increase the recovery of ctDNA molecules for methylation analysis, by combining and optimising reagents from 3 separate kits (see Example 1). Specifically, we have moved away from the most commonly used method of sodium bisulfite conversion for ctDNA methylation analysis and are instead using a non-destructive enzymatic conversion method (NEB EM-seq module). In this component we used only the conversion module (not the whole NEB workflow for EM-Seq) and have modified the experimental processes to increase the recovery of ctDNA fragments (FIG. 3).

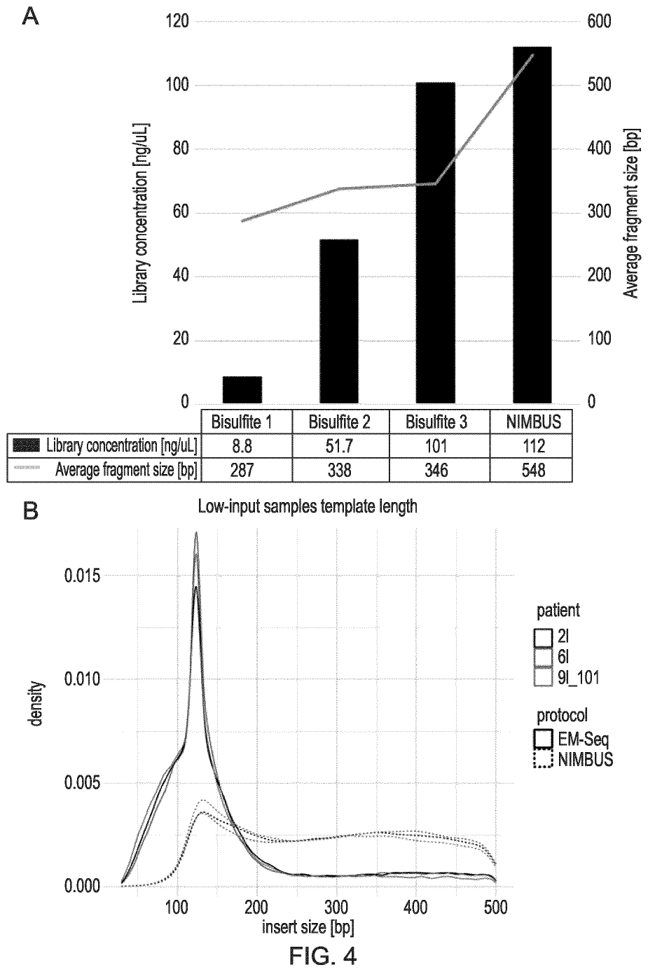

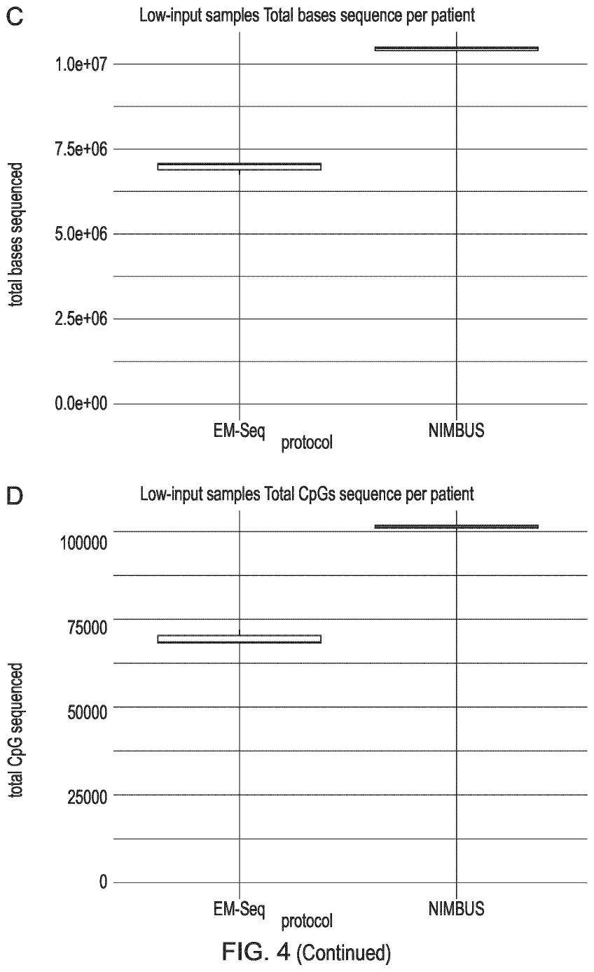

Our data show that A) NIMBUS optimisation of conversion can increase yields compared to the standard cfDNA bisulfite conversion protocol Zymo Lightning (however enzymatic conversion yields remain higher); B) NIMBUS outperforms standard NEB EM-Seq. NIMBUS generates longer fragments therefore retrieving more information on methylation across molecules, and maintains cfDNA fragmentation pattern, consistent with non-destructive DNA methylation conversion; C) NIMBUS cfDNA recovery in low-input samples is 50% higher than EM-Seq; D) NIMBUS protocol recovers more information in form of CpGs. This improves the dynamic range for when comparing with standard EM-Seq (FIG. 4).

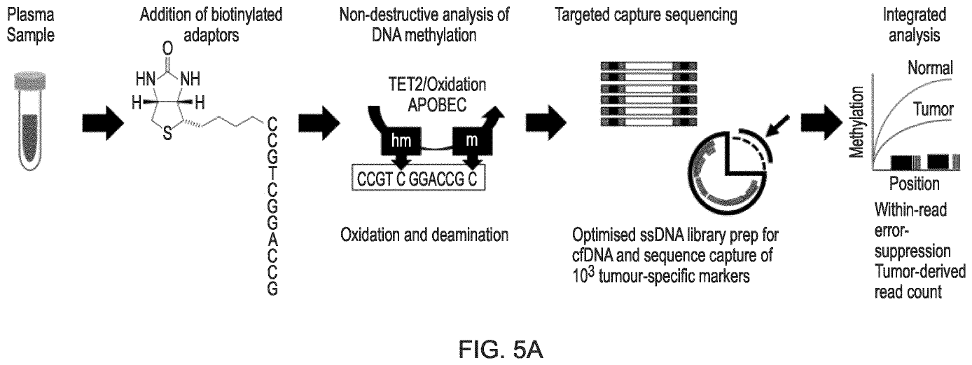

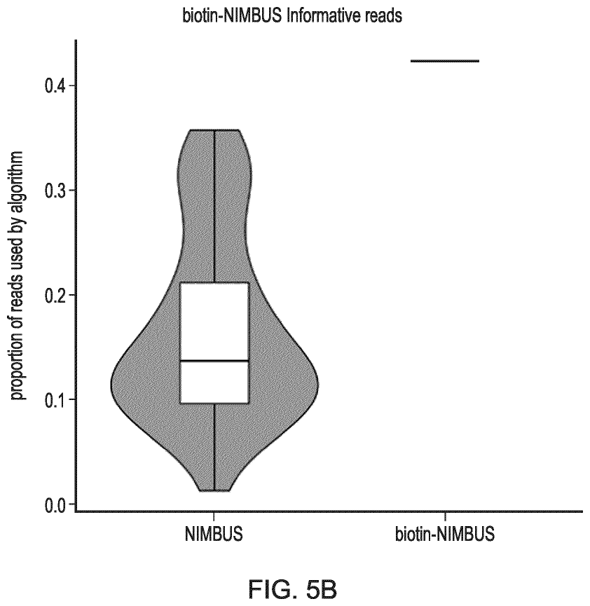

Optionally biotinylated adapters may be used with the method of the present invention to allow solid-phase bead coupling throughout the non-destructive workflow, in order to minimise sample loss during the multiple conversion steps (‘clean-up’) and maintain sample complexity, which was found to result in a further four-fold increase in recovery of informative molecules (FIG. 5).

### 2.3 in Silico NGS Data Analysis Workflow: Scoring System

### Background Methylation.

In order to detect rare methylation events in cfDNA we measure information content for each CpG in the regions of interest in the healthy individuals. To obtain the result we apply rule of succession to determine underlying information content of methylation events characteristic to disease states.

We ignore in further calculations any CpGs that occur in healthy controls above a specified threshold. For example, for prostate cancer cohort we ignore all the CpG for which information content is below 4.32 Shannons.

### Read Rating

For each sequencing read we generate vector of methylation states and we calculate Read Ratings.
a) For standard read rating we sum information content of all methylation states that are concordant with changes observed in diseased tissue and we ignore all the methylation states opposite to ones observed in diseased tissue.
b) For penalised read rating we sum information content of all methylation states that are like changes observed in diseased tissue and we subtract information content of all the methylation states opposite to ones observed in diseased tissue

### Tumour Specific Reads.

We define tumour specific reads as reads that cross an information read rating threshold for given DMR.

The read rating threshold could be:
a) Constant i.e. 0 or 4.32
b) DMR specific threshold based on distribution of read ratings in the healthy controls:
   a. Mean of the Read Ratings for given DMR+multiplicity of standard deviations of the Read Rating for given DMR
   b. Read Rating above certain quantile of the Read Ratings in the given DMR i.e. 99.99^{th} quantile.

We define tumour-specific read as a read that passes one of these thresholds. Counting the number of tumour-specific reads is a diagnostic marker for disease or disease stage.

### NIMBUS Score.

NIMBUS score is a sum of all Read Ratings that pass selected threshold. This can be used as a diagnostic marker.

### SMUGE

Smuge (simple methylation uniformity gauge) is an indicator of intra-read methylation heterogeneity and could be used as marker of a disease. It approximates the uniformity of the methylation within a read. It is the number of methylation state changes counted along the read. The methylation states can be measured depending on the length of the similar methylation state. For minimal stage change of one smuge is expressed as:

$${smuge}_{k} = {\sum }_{j}^{J - 1} \frac{\vert {r}_{j} - {r}_{j + 1} \vert}{2}$$

Where methylation stages in read are encodes as:

$$\vec{{r}_{k}} = [ {r}_{1} , … , {r}_{j} ] , where {r}_{i} ∈ ( - 1 , 1 )$$

Where −1 is unmethylated cytosine and 1 is methylated cytosine.

SMUGE increases for disease states in cfDNA. The mean SMUGE of tumour-specific reads can be used as marker of disease.

Combined SMUGE and Read Rating score SURGE? (SMUGE and Read Rating ToGEther)

Information content of SMUGE can be combined with Read Rating. These reads above SURGE thresholds can be used as markers of disease.

### Predicting Disease Stage.

We can determine disease stage for patients with positive NIMBUS score or significant number of tumour-specific reads. In these patients loci/DMRs with detectable tumour-specific reads are counted. Elastic net model of these loci/DMRs is used to determine stage of the disease.

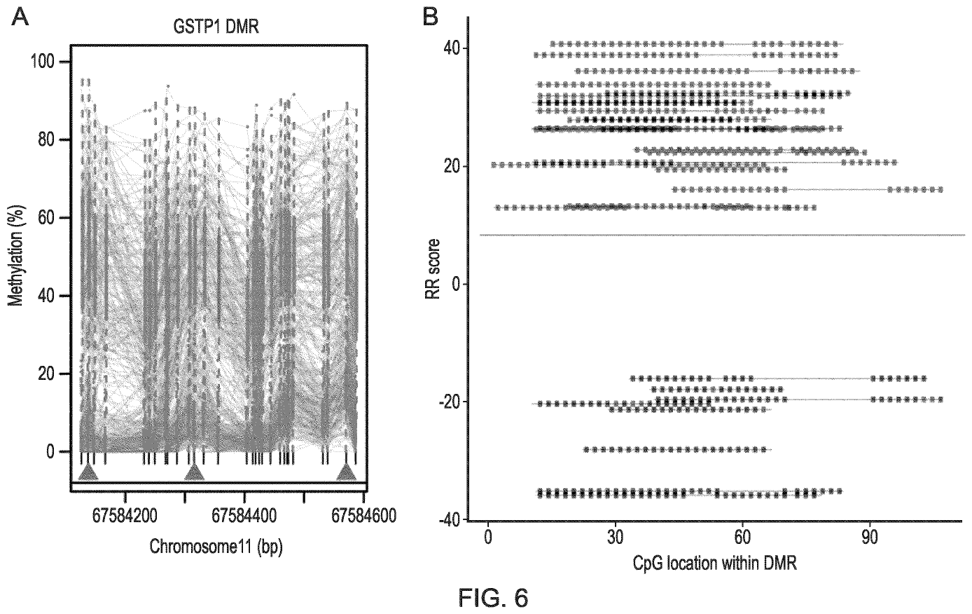

The rare read rating (RRR) (or simply read rating (“RR”)) and smuge scores were calculated separately for each sequencing read derived from these plasma cell-free DNA (cfDNA) samples. Firstly, the RRR score determines if the methylation observed within a read is similar to the signal we observed in previously analysed tumour biopsies (directional patterns of methylation signal). We compare each cytosine in CpG context in a read sequence with methylation changes in cancer. If the read methylation is consistent with cancer tissue, the RRR score is increased. When the methylation change is opposite to the one observed in tissue, the RRR score of the read is decreased (penalised scoring) (FIG. 6).

Smuge analyses the consistency of methylation within each read. Within a given sequencing read CpG methylation can exist in two states (methylated or unmethylated) and in this score we count the switches between these methylation states along each sequencing read (within-read smuge score).

RRR and smuge scores can be combined to create an overall RRR score that integrates ctDNA methylation signals across 10²-10⁴ tumour-specific markers.

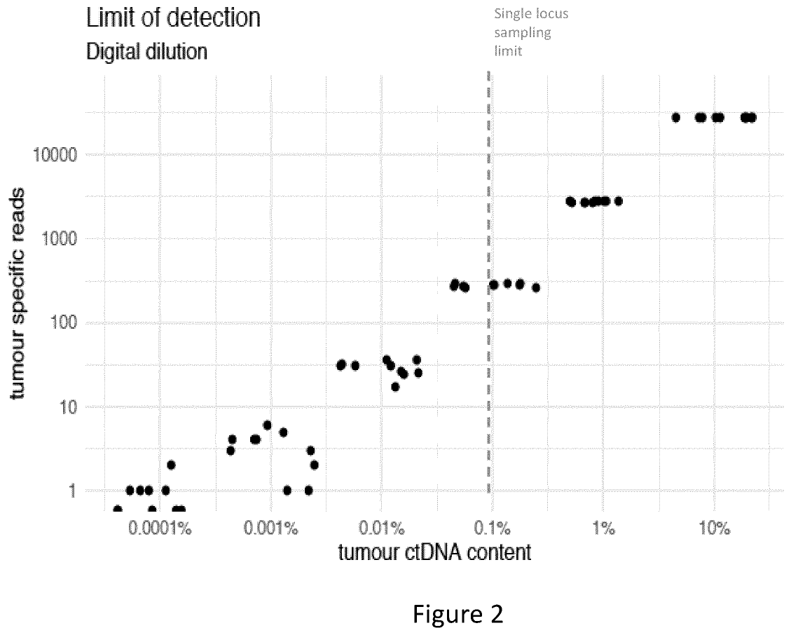

Initial results suggest that we can achieve detection at parts-per-hundred thousand in samples with only a thousand genome equivalents (~100× lower than hard sampling limits—FIG. 2), a range consistent with estimates for many early cancers.

This score allows us to distinguish cancer patients, with detection limits of at least 1-in-10⁴ background (overcoming the limitation of ~1000 input genome equivalents).

### 2.3 Prostate Cancer

NIMBUS was developed using a prostate cancer set (plasma cell-free DNA from 53 benign and 8 metastatic patients, 20 early stage cancers, 20 advanced cancers).

We applied our optimised, targeted methylation sequencing method that leverages non-destructive conversion, optimised library preparation and targeted sequence capture of 8551 differentially methylated regions. Sequencing was performed on an Illumina sequencing platform.

We have identified 8551 tumour-specific DNA methylation markers for prostate cancer (see Table 4A). These tumour-specific features were defined through our analysis of non-metastatic prostate tumour and benign tissue from published data and our in-house datasets. TO select the tumour-specific markers in blood plasma cfDNA (where multiple tissue types may be represented), we have analysed a panel of 8551 DNA methylation markers (covering 3.5Mbp) in cfDNA samples from healthy controls, cancer-free men with symptoms of prostate cancer and men with metastatic prostate cancer. Using this core set of ~10³ tumour-specific markers we have performed proof of concept analysis on limiting mixture experiments to define the sensitivity of our workflow. Currently, in digital mixture dataset (using plasma cfDNA from healthy control and metastatic prostate cancer patients) we are detecting clear signal at 1-2 orders of magnitude below the best single-locus assays (e.g. ddPCR, achieving detection at 1:10,000 to 1:100,000 from only 10³ genomic equivalents (FIG. 2)). This is only possible because each of our 8551 tumour-specific methylation markers are represented by 10² informative sequencing reads.

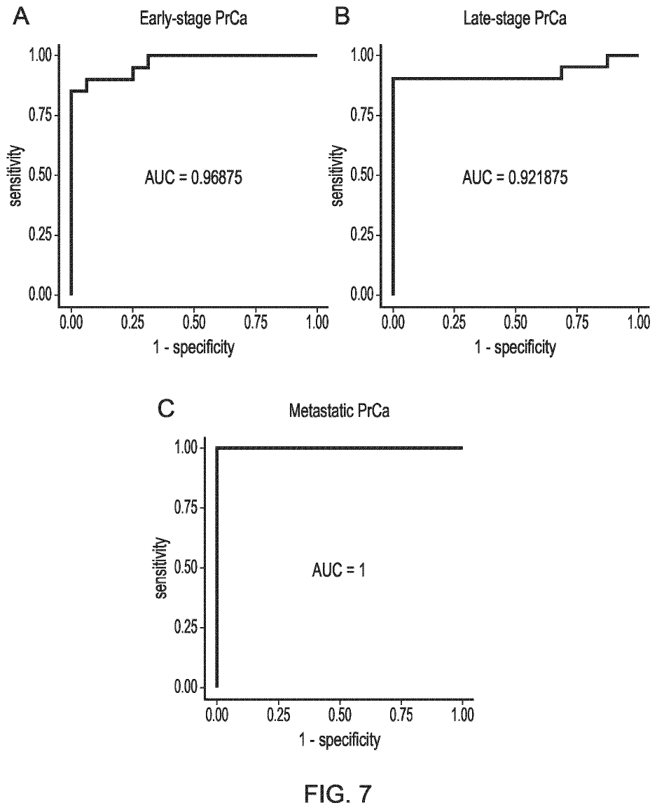

The controls used for comparisons in our assessment of NIMBUS detection specificity (ROC curve AUC analysis) are men with raised PSA and MRI suspicion of cancer, representing an ideal real-world control cohort that would need to be discriminated from cases in the clinical setting (FIG. 7). In some embodiments, the method of the invention may be applied to a subject or population already considered at risk of having or developing a particular cancer. This may allow for greater sensitivity and/or specificity as compared with tests typically applied to a general population and which are aimed at pan-cancer detection.

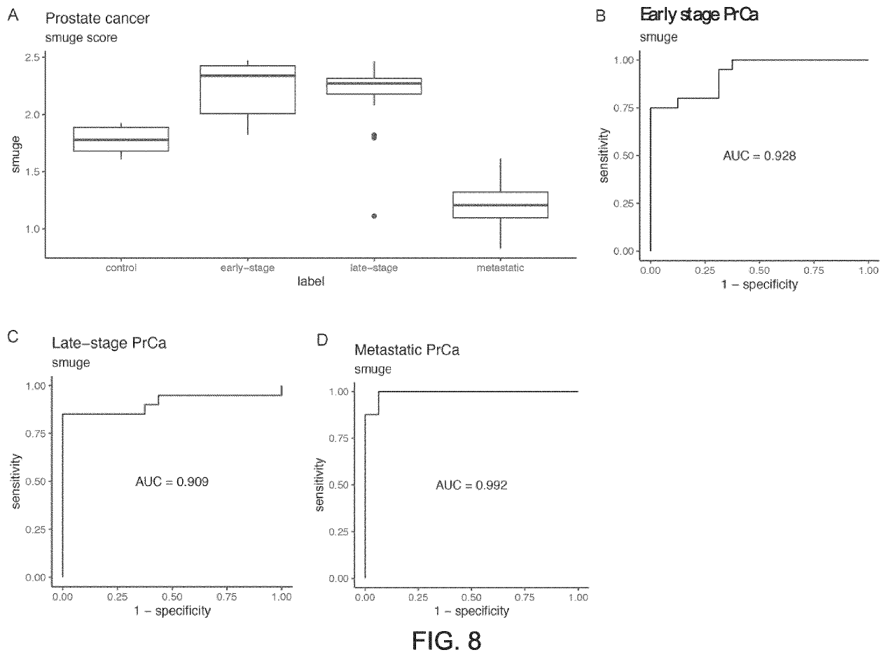

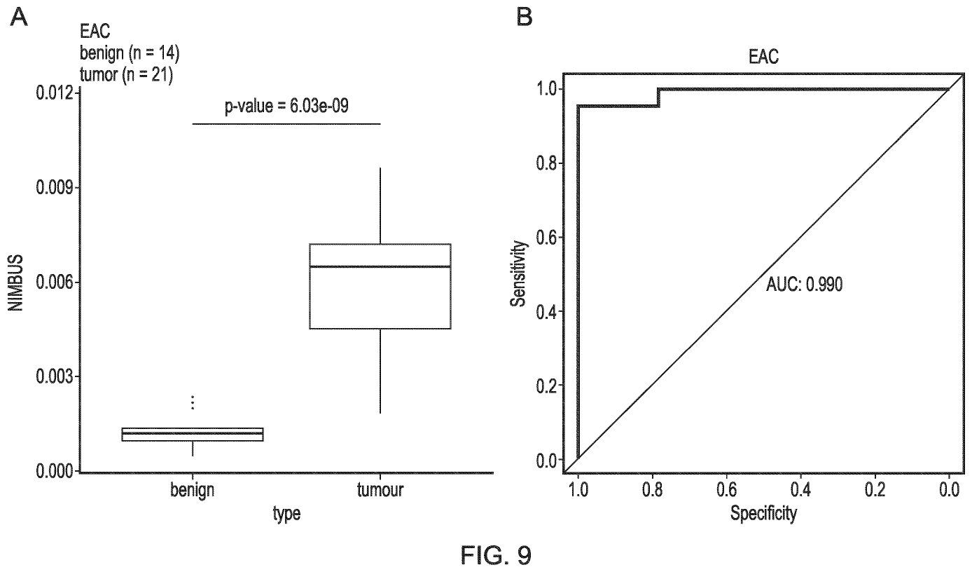

We observe high smuge scores in plasma ctDNA from early and advanced stages of the prostate cancer (FIG. 8A-D). Smuge, the measurement of heterogeneity of methylation, can be use as stand-alone marker to distinguish prostate cancer from controls. However, its relationship is inverse for metastatic prostate cancer (FIG. 8A) which makes the test inverse for this stage of cancer.

Nimbus was found to detect 85% of stage I prostate cancers at 95% specificity. Staging early and late stage has 75% accuracy when using lasso regression.

Previously proposed tests (Klein et al. 2021) has overall 11% sensitivity for PrCa, 3% for stage I, and 5% for stage II, 14% stage III, 83% stage IV) on 420 PrCa (Prostate Cancer) patients.

### SUMMARY

The present examples have established:
An efficient, ultra-low input method for DNA methylation analysis (wet lab).
A novel scoring platform that leverages features across thousands of methylation markers (dry lab).
Comparison of current gold standard methodology (bisulfite conversion) with NIMBUS method showing improved recovery of cfDNA and higher yields for the libraries with NIMBUS.
A detection limit of 1-2 orders of magnitude below the best single-locus assays i.e. 10⁻⁴-10⁻⁵ for prostate cancer ctDNA measurement for ctDNA detection.
The method has been applied on 101 prostate cancer samples. Nimbus enabled PrCa diagnosis and staging.

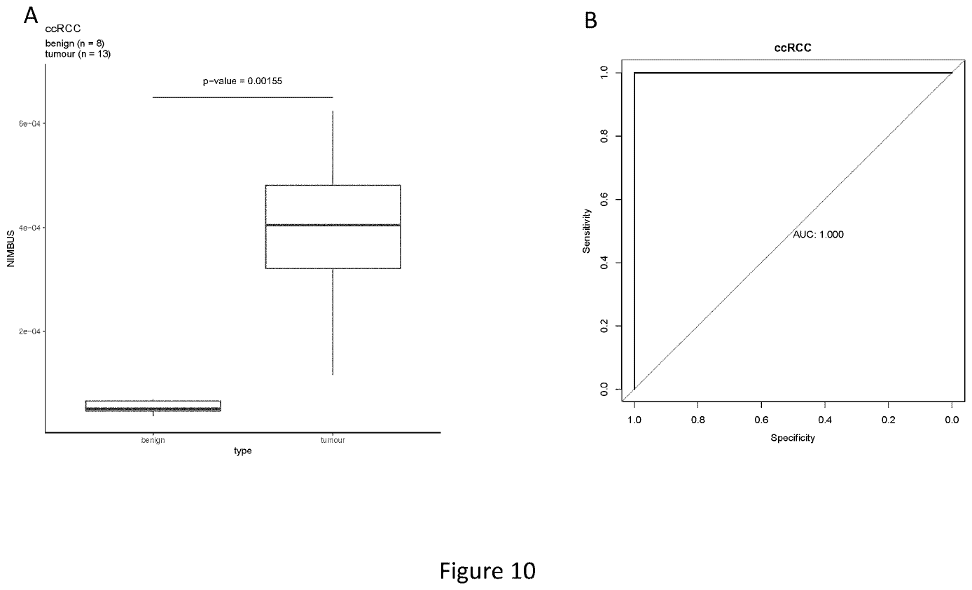

Certain improvements are evident in comparison to the results described in Rossi (2022). In particular, comparing FIG. 10 (AUC=1.000) for ccRCC with FIG. 7.5, panel D of Rossi (2022) (AUC=0.96), the method of an embodiment of the present invention demonstrates higher performance for benign vs. tumour of ccRCC samples. Moreover, the following observations are relevant to the comparison with Rossi (2022):
1) Separation of cases is improved in the embodiment of the present invention: median tumour cases as expressed by benign signal is 2× for FIG. 7C of Rossi (2022) and 8× for FIG. 10;
2) Dynamic range of the signal (as ratio between lowest signal and highest signal) is higher for FIG. 10 of the present application (4× for Rossi (2022) vs 16× lowest benign signal for FIG. 10);
3. At 100% specificity the sensitivity for Rossi (2022) is 66.7% and it is 100% for FIG. 10 of the present application.

### Treatment Response Prediction

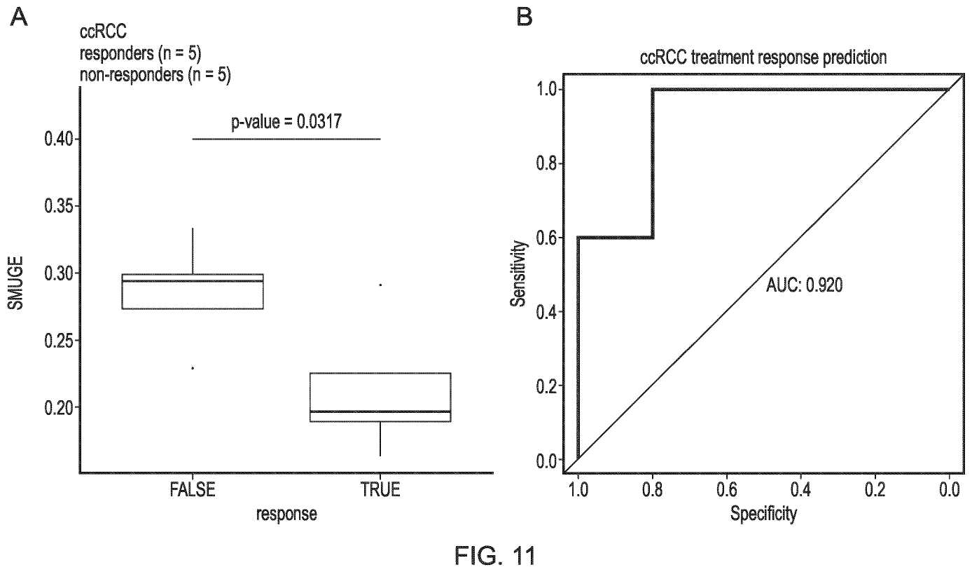

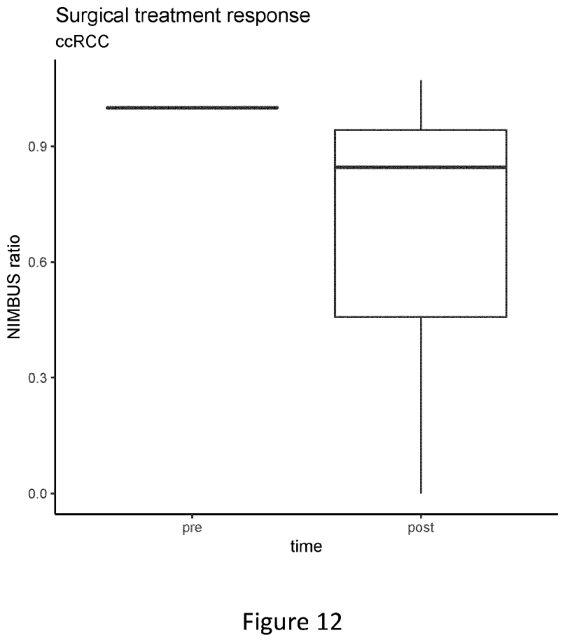

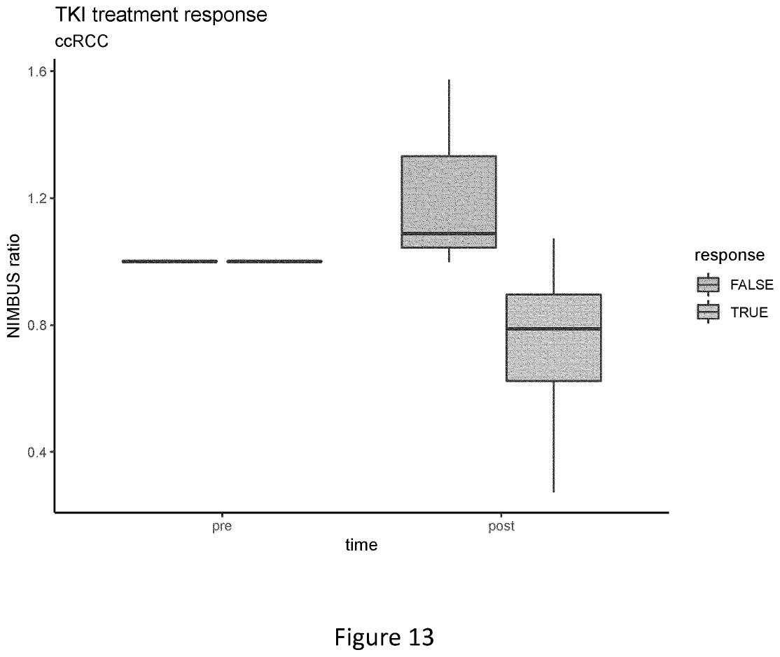

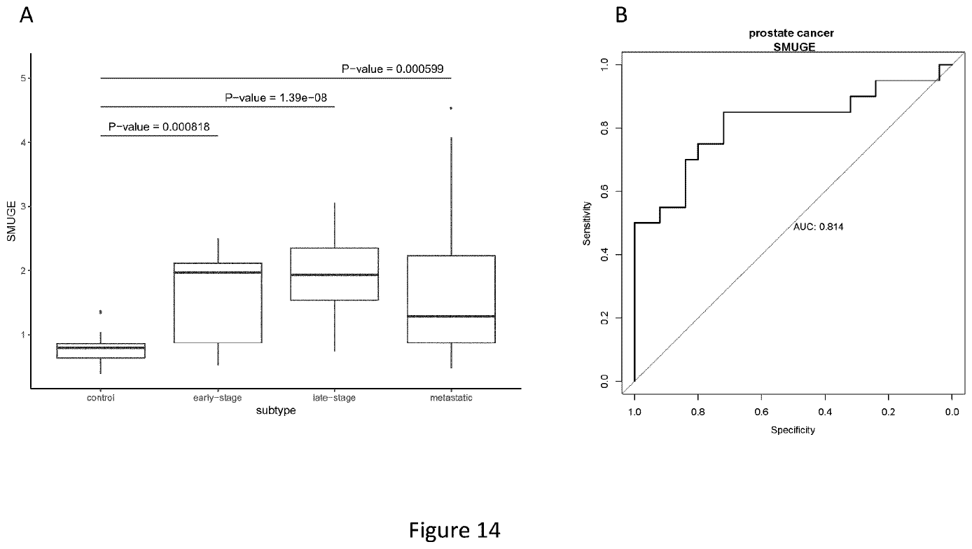

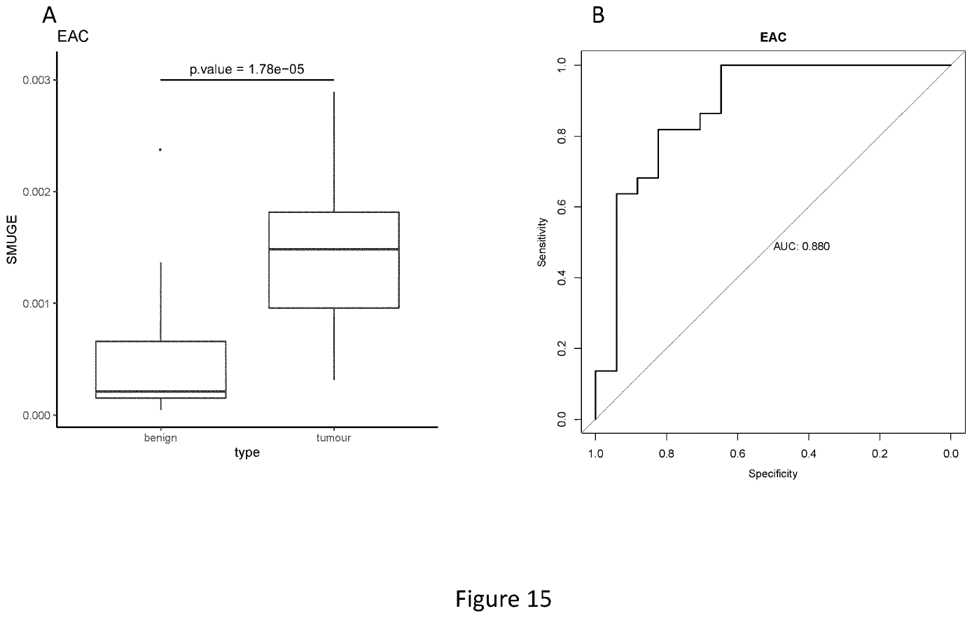

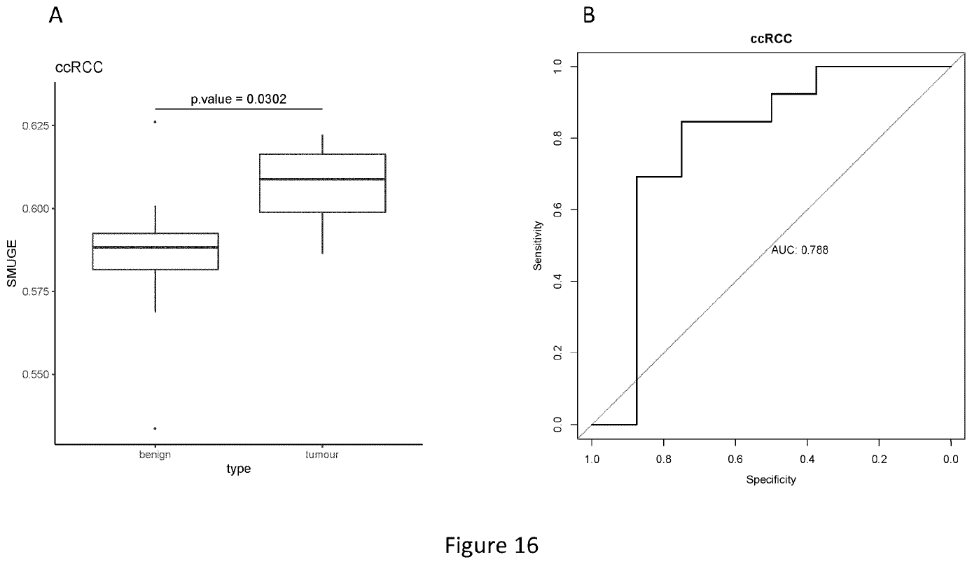

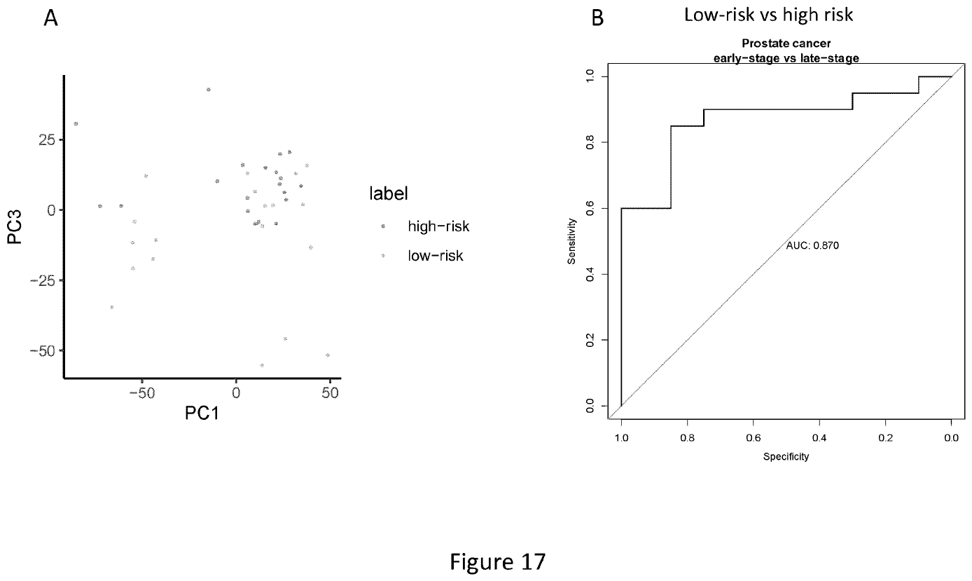

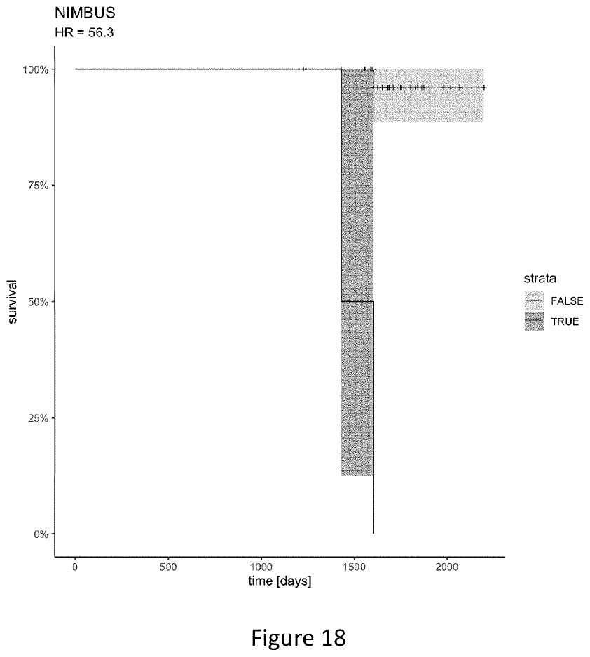

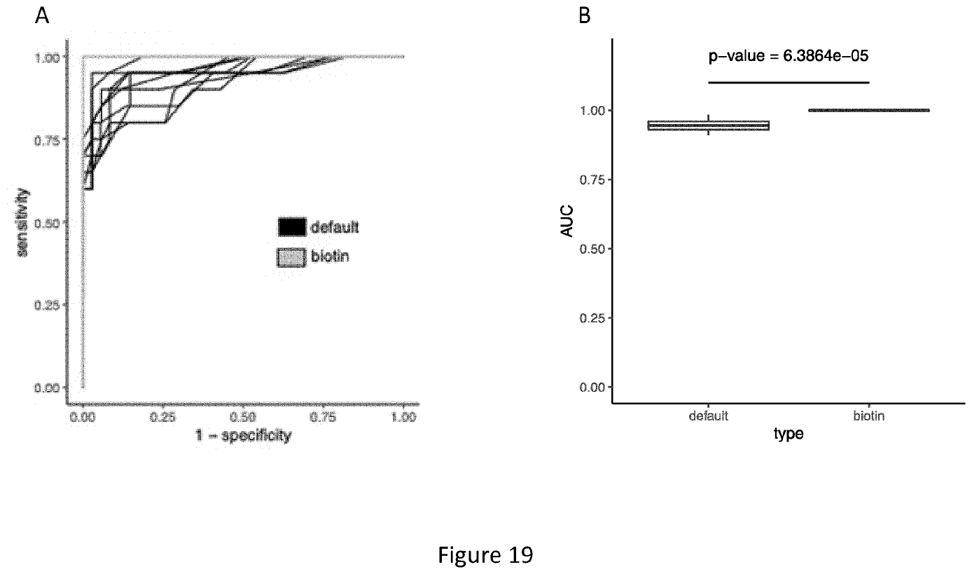

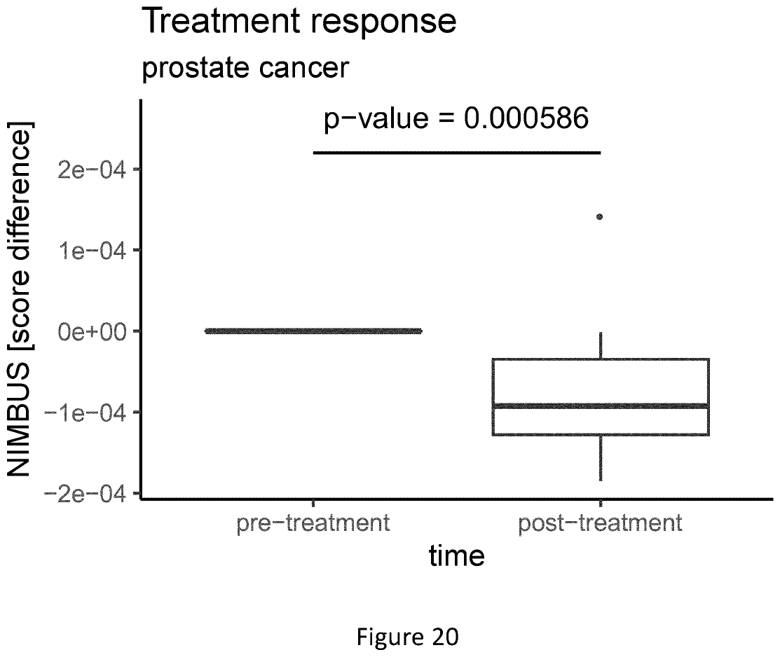

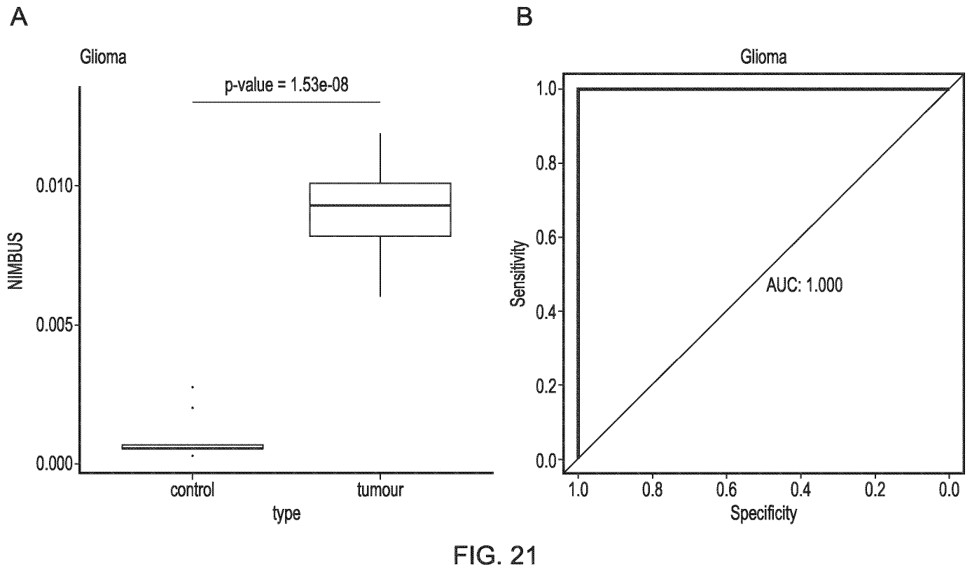

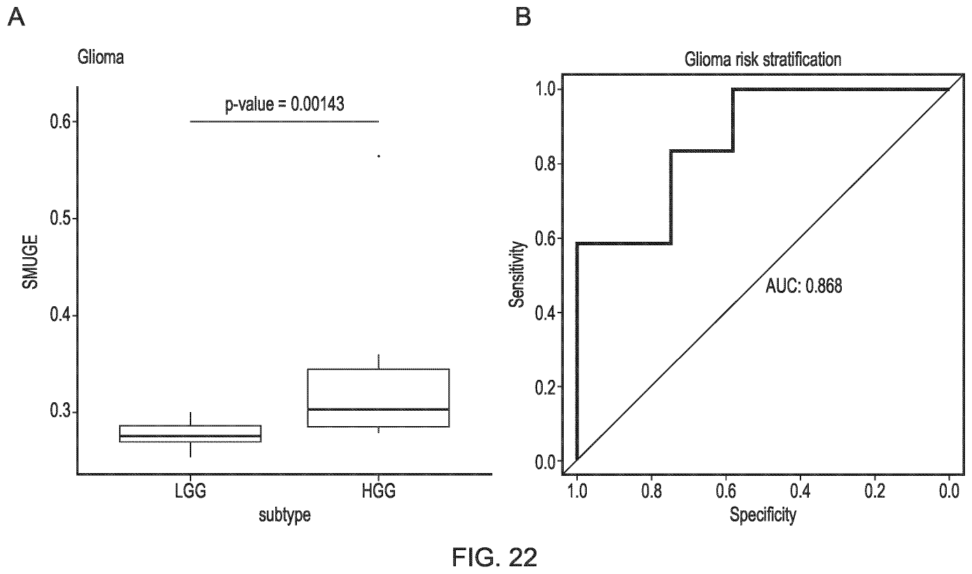

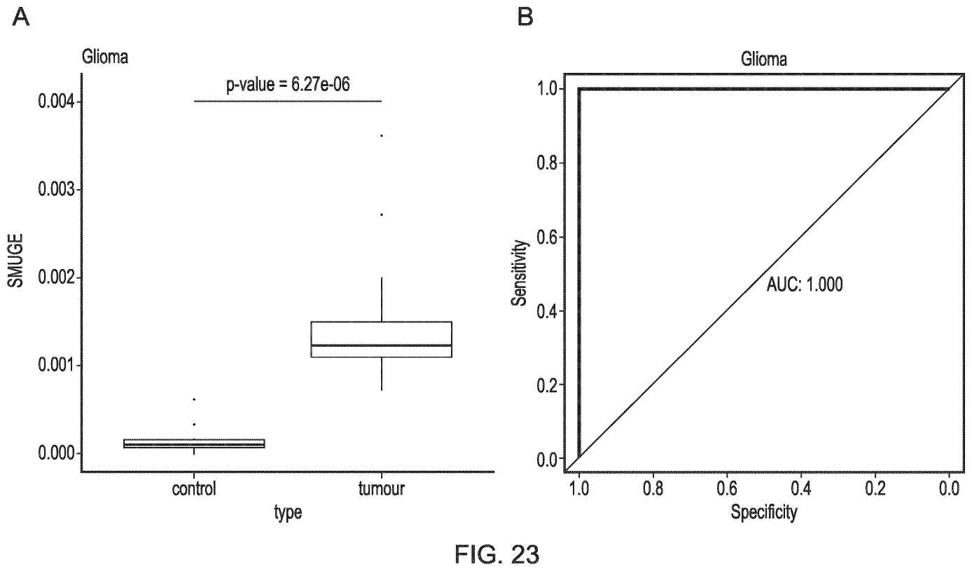

Treatment types for ccRCC patients analyzed in treatment response prediction (see FIG. 11):

TABLE 5

| Treatment | Response to treatment | Number of patients |
|---|---|---|
| Cabozantinib | FALSE | 1 |
| Cabozantinib | TRUE | 4 |
| Lenvatinib + Everolimus | FALSE | 1 |
| Pazopanib | TRUE | 1 |
| Tivozanib | FALSE | 3 |

Responders (n=5)
Non-responders (n=5)
Cabozantinib (n=5)
Lenvatinib+Everolimus (n=1)
Pazopanib (n=1).
Tivozanib (n=1)

TABLE 1
ccRCC

| | | |
|---|---|---|
| 1:851356-852277 | 1:860799-861363 | 1:909497-910112 |
| 1:970547-971374 | 1:1006631-1007252 | 1:1067439-1068372 |
| 1:1591485-1591832 | 1:1599115-1599470 | 1:1600551-1600965 |
| 1:2789222-2790150 | 1:2857604-2857932 | 1:2862283-2862768 |
| 1:2961693-2963293 | 1:2968523-2968833 | 1:3002011-3002403 |
| 1:3090925-3091470 | 1:3120885-3121186 | 1:3156796-3157489 |
| 1:3188243-3188839 | 1:3650679-3651490 | 1:3718184-3718671 |
| 1:3746250-3746755 | 1:3748368-3749020 | 1:6127910-6128220 |
| 1:6184365-6184770 | 1:6447595-6448321 | 1:6958429-6958868 |
| 1:7613496-7613917 | 1:8571512-8571848 | 1:9069732-9070187 |
| 1:9183359-9183839 | 1:9689938-9690381 | 1:10161542-10161934 |
| 1:10599777-10600078 | 1:10627332-10627712 | 1:10692543-10692886 |
| 1:10706287-10706961 | 1:10963966-10964305 | 1:11318047-11319320 |
| 1:11479703-11480499 | 1:11653116-11654374 | 1:11842721-11844115 |
| 1:11877873-11878605 | 1:12063349-12064235 | 1:12478209-12478622 |
| 1:12739273-12739966 | 1:13512503-13513417 | 1:13700332-13700763 |
| 1:15008113-15009459 | 1:15258681-15259490 | 1:15488896-15489697 |
| 1:15757829-15759716 | 1:15797571-15797932 | 1:15897718-15898091 |
| 1:16048076-16048559 | 1:16226593-16227379 | 1:16513344-16513766 |
| 1:16520491-16521546 | 1:16544553-16544854 | 1:16647239-16648768 |
| 1:16692726-16693906 | 1:16727120-16728647 | 1:16758677-16760498 |
| 1:16927794-16928273 | 1:17189379-17190348 | 1:17191471-17192815 |
| 1:17606904-17607343 | 1:18632257-18633282 | 1:18947155-18947504 |
| 1:18965763-18966193 | 1:19087716-19088095 | 1:19665543-19665999 |
| 1:19665849-19666479 | 1:19765491-19765829 | 1:20847438-20847903 |
| 1:21337975-21338833 | 1:21427846-21428953 | 1:21622032-21622391 |
| 1:22128271-22129695 | 1:22345115-22346405 | 1:22350061-22350362 |
| 1:22780474-22781082 | 1:22832029-22832485 | 1:23225183-23225579 |
| 1:23459646-23460912 | 1:23801328-23801659 | 1:23902521-23902876 |
| 1:24187782-24188272 | 1:24365280-24365705 | 1:24409620-24409942 |
| 1:24930713-24932197 | 1:25587337-25587860 | 1:25618056-25618553 |
| 1:25771574-25772022 | 1:26093589-26095086 | 1:26161097-26161534 |
| 1:26219362-26219704 | 1:26302873-26303587 | 1:28578749-28580177 |
| 1:28756962-28757284 | 1:29146968-29147457 | 1:29350116-29350417 |
| 1:30342291-30342758 | 1:32204860-32205407 | 1:32426946-32427842 |
| 1:32782045-32782346 | 1:32925783-32926280 | 1:33306647-33307653 |
| 1:34176537-34177514 | 1:35573605-35574482 | 1:36008578-36008952 |
| 1:36321263-36322331 | 1:36374048-36375182 | 1:36568732-36569033 |
| 1:37545084-37545426 | 1:37556852-37557242 | 1:37735099-37735556 |
| 1:37764060-37765361 | 1:38045080-38046079 | 1:38474994-38477037 |
| 1:38559358-38559899 | 1:38578331-38578818 | 1:38815743-38816204 |
| 1:39082096-39082437 | 1:39268369-39268936 | 1:39683560-39684273 |
| 1:40654093-40654593 | 1:40884302-40884771 | 1:41632035-41632337 |
| 1:41698854-41699431 | 1:41795402-41795874 | 1:42145708-42146170 |
| 1:43582054-43582448 | 1:43933047-43933720 | 1:44031548-44031872 |
| 1:45172032-45172425 | 1:45327221-45327809 | 1:46166869-46167613 |
| 1:46301605-46303956 | 1:46328002-46328328 | 1:46328734-46329095 |
| 1:46447886-46448616 | 1:46467022-46467480 | 1:46485416-46485918 |
| 1:46520494-46520961 | 1:46543625-46544404 | 1:47023374-47023840 |
| 1:47416406-47416987 | 1:47434019-47434863 | 1:47439430-47440691 |
| 1:47443892-47445018 | 1:47575836-47576297 | 1:50048354-50048893 |
| 1:50326559-50327132 | 1:50414953-50416138 | 1:50421551-50422878 |
| 1:50424520-50425305 | 1:50427648-50428281 | 1:51583550-51583898 |
| 1:51644562-51644949 | 1:51736969-51737297 | 1:52142809-52143326 |
| 1:52920743-52921243 | 1:53062676-53063423 | 1:54228578-54230011 |
| 1:54354963-54356581 | 1:56577328-56578155 | 1:58576240-58576719 |
| 1:58577287-58577923 | 1:59360082-59360533 | 1:59707082-59707414 |
| 1:61287165-61288304 | 1:62194687-62195482 | 1:63318081-63321650 |
| 1:63324144-63324950 | 1:64385017-64385386 | 1:65195958-65196372 |
| 1:65525216-65526146 | 1:67134403-67135240 | 1:67389954-67390402 |
| 1:69759771-69760207 | 1:73593157-73593479 | 1:75124571-75125980 |
| 1:75130085-75131278 | 1:75132446-75133434 | 1:75134962-75135880 |
| 1:75136548-75137391 | 1:77866250-77866620 | 1:79006448-79007024 |
| 1:84998002-84998851 | 1:85277900-85279549 | 1:85598919-85599220 |
| 1:85615921-85616535 | 1:86154755-86155267 | 1:86156710-86157338 |
| 1:88462917-88463350 | 1:88983733-88984118 | 1:89566842-89568553 |
| 1:89738770-89739849 | 1:90716421-90716737 | 1:90716534-90717882 |
| 1:90719420-90720139 | 1:90724906-90727487 | 1:90834484-90836325 |
| 1:94739628-94740043 | 1:98045262-98046018 | 1:101309476-101310108 |
| 1:101786465-101787016 | 1:101787199-101787500 | 1:101846891-101847205 |
| 1:108041550-108042009 | 1:108721422-108721977 | 1:109398347-109399284 |
| 1:109821087-109822834 | 1:109826920-109827328 | 1:109892984-109893306 |
| 1:110068069-110069241 | 1:110084007-110085043 | 1:110209388-110209809 |
| 1:110211754-110212194 | 1:110606019-110606448 | 1:110673940-110674679 |
| 1:110675432-110675825 | 1:111271014-111271861 | 1:112707552-112708000 |
| 1:114151948-114152433 | 1:114153968-114154731 | 1:114854624-114855346 |
| 1:115168672-115169108 | 1:115828810-115829516 | 1:115837589-115838421 |
| 1:116167388-116167924 | 1:116758436-116758806 | 1:116816394-116817897 |
| 1:117122452-117122786 | 1:118979541-118980500 | 1:118983363-118984593 |
| 1:118986315-118987081 | 1:118989178-118989675 | 1:118992935-118993746 |
| 1:118998999-118999938 | 1:119006014-119008465 | 1:119605087-119605486 |
| 1:120388473-120388847 | 1:121009757-121010200 | 1:121048338-121048954 |
| 1:125085943-125087998 | 1:143661734-143662044 | 1:144456359-144456826 |
| 1:144490481-144490943 | 1:145453871-145454453 | 1:147052068-147052506 |
| 1:147072300-147072780 | 1:147077570-147079649 | 1:147080585-147081203 |
| 1:148048588-148049411 | 1:148060132-148060491 | 1:148244745-148245046 |
| 1:148317387-148318109 | 1:149098260-149098677 | 1:149740275-149740665 |
| 1:149746891-149747955 | 1:150282661-150283168 | 1:150484435-150484736 |
| 1:151131329-151132831 | 1:151133651-151133966 | 1:151326041-151326580 |
| 1:151513098-151514235 | 1:151970636-151970973 | 1:152037147-152039032 |
| 1:152107453-152109502 | 1:152623337-152623814 | 1:152784737-152785275 |
| 1:153057314-153058269 | 1:153113317-153114537 | 1:153202521-153203517 |
| 1:154430980-154433523 | 1:154502706-154503408 | 1:154868264-154869797 |
| 1:155294475-155296006 | 1:155940582-155940886 | 1:156222732-156223033 |
| 1:156642199-156642934 | 1:156646105-156647228 | 1:156675565-156676844 |
| 1:156844943-156845900 | 1:157184409-157185429 | 1:157194966-157195608 |
| 1:157978587-157979485 | 1:159005752-159006173 | 1:159899963-159900598 |
| 1:159946031-159946600 | 1:160084331-160084940 | 1:160692272-160692898 |
| 1:160742203-160744743 | 1:161098773-161099544 | 1:161457612-161458879 |
| 1:162009860-162010162 | 1:162028622-162029125 | 1:163307377-163307826 |
| 1:164320769-164321837 | 1:164602735-164603210 | 1:165354825-165355772 |
| 1:166602304-166602613 | 1:166883868-166884794 | 1:167439073-167440121 |
| 1:167516390-167518799 | 1:168266612-168267109 | 1:169161833-169162222 |
| 1:169460351-169461170 | 1:170661167-170662503 | 1:171604004-171604411 |
| 1:172909505-172910406 | 1:172933717-172934062 | 1:175079488-175080528 |
| 1:175471134-175471435 | 1:175599135-175599822 | 1:177029420-177030049 |
| 1:179310387-179310955 | 1:179591347-179592021 | 1:179639691-179640114 |
| 1:181141533-181141967 | 1:181481840-181484153 | 1:182056754-182057498 |
| 1:182128511-182129043 | 1:182952497-182953070 | 1:183385805-183386106 |
| 1:183416663-183417571 | 1:183891481-183891816 | 1:184626053-184626417 |
| 1:196875011-196875588 | 1:196877892-196878395 | 1:197917910-197918811 |
| 1:197921200-197921864 | 1:198155325-198155626 | 1:198677686-198679984 |
| 1:199243567-199243943 | 1:200872979-200873341 | 1:200873308-200874039 |
| 1:201275205-201275759 | 1:201283657-201284560 | 1:201476484-201476907 |
| 1:201614322-201614626 | 1:201647671-201648119 | 1:201648699-201650923 |
| 1:201739133-201739581 | 1:201740059-201740428 | 1:202488402-202488760 |
| 1:203029002-203029303 | 1:203075348-203076326 | 1:205313716-205314412 |
| 1:205581996-205582473 | 1:205935027-205936299 | 1:206056051-206056512 |
| 1:206802372-206803589 | 1:207495827-207496140 | 1:207496018-207496908 |
| 1:207668809-207669472 | 1:208007772-208008207 | 1:208697288-208697731 |
| 1:209231352-209232278 | 1:209353996-209354462 | 1:209431610-209432330 |
| 1:209805197-209805784 | 1:212469975-212470330 | 1:212514430-212515871 |
| 1:213982943-213983744 | 1:214187431-214187899 | 1:216723361-216724235 |
| 1:217137286-217138493 | 1:218156616-218157011 | 1:220813040-220813577 |
| 1:220877190-220877997 | 1:220891399-220892402 | 1:220894440-220896963 |
| 1:222619062-222619506 | 1:223740212-223740560 | 1:224175550-224176056 |
| 1:224341026-224341471 | 1:224542627-224543481 | 1:224616933-224618301 |
| 1:225677218-225677852 | 1:225887472-225888218 | 1:226061241-226061750 |
| 1:226370584-226370954 | 1:226679765-226680256 | 1:227319085-227319437 |
| 1:227541525-227542027 | 1:227560763-227561379 | 1:227778627-227779023 |
| 1:227786248-227787154 | 1:228107598-228108688 | 1:228211820-228213065 |
| 1:228276358-228276937 | 1:228457356-228458578 | 1:228464145-228464928 |
| 1:228469367-228470010 | 1:228935334-228935741 | 1:228998268-228998569 |
| 1:229431381-229433625 | 1:229717606-229717961 | 1:230987022-230987393 |
| 1:231041037-231041451 | 1:231161050-231161670 | 1:231163063-231164125 |
| 1:232590942-232591367 | 1:232599451-232600784 | 1:233588211-233588612 |
| 1:234206778-234207172 | 1:234874632-234875117 | 1:234917999-234918356 |
| 1:235650682-235651417 | 1:236159794-236160239 | 1:236168447-236169012 |
| 1:237041788-237042228 | 1:237042029-237042332 | 1:238469395-238469881 |
| 1:239084237-239084722 | 1:241356822-241357792 | 1:241424211-241424628 |
| 1:241532975-241533834 | 1:241612584-241612984 | 1:241944841-241945142 |
| 1:242188597-242189790 | 1:242890192-242890981 | 1:243482835-243483639 |
| 1:244348990-244349689 | 1:245130686-245130988 | 1:245585698-245586062 |
| 1:245823163-245823572 | 1:246788764-246789843 | 1:247526647-247527960 |
| 1:247856983-247858175 | 10:379737-380549 | 10:1160407-1160938 |
| 10:1463103-1463582 | 10:1464086-1465664 | 10:1556297-1556902 |
| 10:1594938-1595239 | 10:1678657-1679363 | 10:1737065-1737518 |
| 10:2772674-2773277 | 10:3740425-3740962 | 10:3758119-3758582 |
| 10:3762885-3763400 | 10:3865473-3866719 | 10:3887673-3888663 |
| 10:3906413-3907165 | 10:4005983-4007254 | 10:4120486-4122091 |
| 10:4344513-4344953 | 10:4383829-4384558 | 10:4459663-4459971 |
| 10:5861370-5861754 | 10:6280279-6280665 | 10:7096664-7097349 |
| 10:7119329-7119870 | 10:7269344-7269776 | 10:7319025-7320388 |
| 10:7475567-7476198 | 10:7561481-7562078 | 10:8035198-8037459 |
| 10:8046768-8047092 | 10:8051291-8052756 | 10:8137486-8138847 |
| 10:8435025-8435534 | 10:9330941-9331415 | 10:11275640-11276148 |
| 10:11437124-11437425 | 10:12043808-12044300 | 10:12077556-12078013 |
| 10:13345387-13346718 | 10:13704581-13706428 | 10:13866453-13866754 |
| 10:14009416-14010268 | 10:14571868-14572776 | 10:14774613-14775479 |
| 10:14809252-14809638 | 10:15254583-15254950 | 10:16519244-16521697 |
| 10:16965911-16966547 | 10:17950374-17951820 | 10:18139901-18141157 |
| 10:18291223-18291851 | 10:21509876-21510896 | 10:22336587-22337096 |
| 10:22344923-22346375 | 10:22475412-22476848 | 10:22477424-22478126 |
| 10:23172160-23174237 | 10:23190911-23192197 | 10:23194962-23195662 |
| 10:23200771-23201074 | 10:24237667-24239066 | 10:24446786-24447136 |
| 10:25060103-25060553 | 10:25175444-25176830 | 10:26211530-26212370 |
| 10:26217311-26217977 | 10:26218301-26218685 | 10:26391638-26392015 |
| 10:26474009-26475798 | 10:26486456-26486867 | 10:26502959-26503391 |
| 10:27746022-27746877 | 10:27958706-27959790 | 10:27999036-27999635 |
| 10:28411105-28412169 | 10:29380704-29381026 | 10:29435831-29436444 |
| 10:29714111-29715605 | 10:30207377-30207696 | 10:30365137-30365711 |
| 10:30503748-30504395 | 10:30638119-30638533 | 10:30704420-30704791 |
| 10:32995566-32995876 | 10:33133864-33134993 | 10:34298435-34298866 |
| 10:34364177-34364503 | 10:34556468-34556951 | 10:42241942-42242800 |
| 10:42897701-42898985 | 10:44878847-44879915 | 10:44896989-44897415 |
| 10:46215415-46215755 | 10:47152658-47152959 | 10:47165753-47166250 |
| 10:48450055-48450525 | 10:49394771-49395375 | 10:49396304-49397384 |
| 10:49608659-49609413 | 10:49610710-49612482 | 10:49613293-49614094 |
| 10:49679190-49679962 | 10:49768153-49769241 | 10:50586257-50586772 |
| 10:52314704-52315217 | 10:52318785-52319480 | 10:52389190-52389504 |
| 10:52689012-52689519 | 10:55597192-55597493 | 10:55627821-55628142 |
| 10:55628444-55628918 | 10:55630284-55631207 | 10:58701196-58701715 |
| 10:60536404-60536902 | 10:61662631-61662937 | 10:62433410-62433836 |
| 10:69452040-69452357 | 10:69483655-69485389 | 10:69572163-69573257 |
| 10:69943824-69944963 | 10:70091090-70091564 | 10:70255215-70255863 |
| 10:70441067-70441613 | 10:70653241-70653617 | 10:70710436-70712467 |
| 10:70963345-70963821 | 10:71805663-71806175 | 10:71831852-71832189 |
| 10:71940026-71940484 | 10:72295882-72297362 | 10:73647507-73648133 |
| 10:75276511-75276812 | 10:75395717-75396338 | 10:75397190-75399868 |
| 10:75405209-75407532 | 10:77592935-77593331 | 10:77620000-77620346 |
| 10:77772810-77773640 | 10:77846966-77847267 | 10:77872476-77872928 |
| 10:78577872-78579425 | 10:79249703-79250847 | 10:79299340-79299827 |
| 10:79358860-79359260 | 10:80367299-80367736 | 10:80429572-80430036 |
| 10:84114081-84114783 | 10:84160401-84160732 | 10:84330040-84330766 |
| 10:84543978-84544407 | 10:86262836-86263579 | 10:86693772-86694268 |
| 10:87084746-87085137 | 10:87211927-87212297 | 10:91887070-91888113 |
| 10:92239154-92239710 | 10:92420024-92420717 | 10:93068123-93069082 |
| 10:93208659-93209142 | 10:95295480-95295963 | 10:96384402-96385016 |
| 10:96396202-96396514 | 10:96919986-96920499 | 10:97164983-97165285 |
| 10:97714795-97715428 | 10:98133220-98133522 | 10:98583175-98583516 |
| 10:98670546-98671169 | 10:99330437-99331875 | 10:99403679-99404021 |
| 10:99526632-99528051 | 10:99537615-99539296 | 10:100114953-100115369 |
| 10:100621417-100621755 | 10:100741747-100742624 | 10:100748938-100750140 |
| 10:101046938-101048807 | 10:101050640-101051174 | 10:101130775-101131674 |
| 10:101134121-101134566 | 10:101224256-101224751 | 10:101227366-101227893 |
| 10:101776430-101776925 | 10:101778570-101779523 | 10:102240495-102241142 |
| 10:102604611-102605056 | 10:102776226-102776527 | 10:102919395-102920014 |
| 10:103277821-103278975 | 10:103584246-103585141 | 10:103652714-103654225 |
| 10:103660987-103661963 | 10:106611787-106612876 | 10:107163917-107164525 |
| 10:109456773-109457529 | 10:110340726-110341205 | 10:111078223-111079451 |
| 10:112226832-112227938 | 10:113788557-113789030 | 10:114044232-114045284 |
| 10:114607660-114608121 | 10:117167045-117167965 | 10:118593999-118596375 |
| 10:119341797-119342337 | 10:119814349-119814650 | 10:122385726-122386642 |
| 10:122441813-122442268 | 10:122704374-122704690 | 10:123134465-123135203 |
| 10:123141791-123142631 | 10:123145902-123146368 | 10:123148843-123150359 |
| 10:123665270-123666999 | 10:123971948-123973369 | 10:124043226-124045013 |
| 10:124301753-124302135 | 10:124341104-124342341 | 10:124447069-124447594 |
| 10:124476147-124476580 | 10:124576556-124576857 | 10:125128727-125129069 |
| 10:125151288-125151905 | 10:126063171-126063686 | 10:126906056-126906978 |
| 10:127346485-127346945 | 10:127907865-127909139 | 10:128285257-128285697 |
| 10:128500246-128501102 | 10:128709390-128710456 | 10:129517783-129518157 |
| 10:129958684-129960670 | 10:130107958-130108457 | 10:131094983-131096430 |
| 10:131307624-131307966 | 10:131310582-131312904 | 10:132045244-132045545 |
| 10:132146540-132146962 | 10:132279839-132280161 | 10:132407849-132408338 |
| 10:132408653-132409228 | 10:132411678-132412228 | 10:132436779-132437239 |
| 10:132544882-132545266 | 10:132547408-132548027 | 10:132784434-132785082 |
| 10:132786257-132786752 | 10:132870475-132870876 | 10:132879300-132879791 |
| 10:133145391-133145915 | 10:133218747-133220769 | 10:133231098-133231800 |
| 10:133237963-133238667 | 10:133263764-133265253 | 10:133304883-133306135 |
| 11:280832-281902 | 11:393281-393582 | 11:504055-505155 |
| 11:1024968-1025498 | 11:1037562-1038074 | 11:1337093-1337813 |
| 11:1359770-1360501 | 11:1652265-1652576 | 11:1705589-1707210 |
| 11:1747571-1748566 | 11:2194423-2194783 | 11:2202569-2202925 |
| 11:2270301-2271305 | 11:2443574-2444379 | 11:2463217-2463810 |
| 11:2768983-2769433 | 11:2791704-2792352 | 11:2868990-2870281 |
| 11:3437396-3437881 | 11:3641688-3642008 | 11:4212070-4212497 |
| 11:4578740-4579246 | 11:4770686-4771322 | 11:5250052-5250829 |
| 11:5679738-5680238 | 11:5937609-5938269 | 11:6783495-6783797 |
| 11:7576373-7577593 | 11:8080615-8081785 | 11:8168455-8169493 |
| 11:8384667-8385181 | 11:9039213-9039574 | 11:12111168-12111610 |
| 11:13215907-13216933 | 11:13717561-13717862 | 11:14971683-14972582 |
| 11:16804739-16805049 | 11:17387756-17388113 | 11:17719374-17720518 |
| 11:17735849-17736889 | 11:18046201-18046607 | 11:18704862-18706116 |
| 11:18705931-18706239 | 11:18987777-18988277 | 11:19242514-19243038 |
| 11:20159399-20160842 | 11:20163018-20163846 | 11:20342821-20343133 |
| 11:20596369-20599575 | 11:20601035-20601675 | 11:22341173-22342068 |
| 11:22432396-22433220 | 11:27719468-27720710 | 11:27722306-27723448 |
| 11:30016076-30017364 | 11:30017114-30017426 | 11:30583754-30584325 |
| 11:31800518-31802021 | 11:31804724-31805569 | 11:31812034-31812837 |
| 11:31823451-31824131 | 11:31824887-31826975 | 11:31987112-31987596 |
| 11:32333089-32333893 | 11:32427554-32429325 | 11:32430450-32431229 |
| 11:32432886-32433967 | 11:32437181-32437717 | 11:33061304-33061751 |
| 11:34243545-34243846 | 11:34523816-34524143 | 11:34600663-34601201 |
| 11:35235906-35237062 | 11:35337157-35337528 | 11:35419662-35420164 |
| 11:36353000-36354351 | 11:43548039-43548443 | 11:43580995-43581688 |
| 11:43583054-43584018 | 11:44309114-44310099 | 11:44591633-44592921 |
| 11:44911701-44912011 | 11:45144684-45145946 | 11:46229218-46229660 |
| 11:46702637-46702978 | 11:47187412-47187911 | 11:47389097-47390283 |
| 11:47589471-47590243 | 11:49048196-49049236 | 11:49208091-49208635 |
| 11:49558248-49558717 | 11:55872597-55873630 | 11:57637675-57638061 |
| 11:57646708-57648341 | 11:57778420-57779397 | 11:59197949-59198382 |
| 11:60280357-60282923 | 11:60514926-60515420 | 11:61273412-61274191 |
| 11:62004506-62005023 | 11:62426698-62428194 | 11:62547453-62547815 |
| 11:62551482-62552058 | 11:62687945-62688546 | 11:63769837-63770257 |
| 11:64000809-64001444 | 11:64247777-64248351 | 11:64643992-64644883 |
| 11:64716056-64716370 | 11:64854595-64855520 | 11:64856314-64857045 |
| 11:65047511-65048611 | 11:65204649-65205028 | 11:65355754-65356506 |
| 11:65481254-65482110 | 11:65548913-65549269 | 11:65555905-65556306 |
| 11:65591719-65593890 | 11:65642126-65642761 | 11:65833186-65833491 |
| 11:65872095-65872678 | 11:66311473-66312086 | 11:66313262-66313569 |
| 11:66315864-66317979 | 11:66420619-66421640 | 11:66728729-66729131 |
| 11:66892253-66892634 | 11:67695718-67696169 | 11:67710716-67711370 |
| 11:67884706-67885367 | 11:67954158-67955117 | 11:67990874-67992055 |
| 11:68855853-68856359 | 11:68878078-68878379 | 11:69011149-69011616 |
| 11:69075511-69075812 | 11:69089198-69089716 | 11:69155617-69155918 |
| 11:69311063-69311805 | 11:69643584-69644080 | 11:69653945-69654660 |
| 11:69985790-69986256 | 11:70020794-70021184 | 11:70144714-70145015 |
| 11:70180966-70181267 | 11:70244724-70245245 | 11:70457171-70457611 |
| 11:70487682-70488099 | 11:70542538-70542973 | 11:71140675-71141251 |
| 11:71489210-71489659 | 11:71607242-71608317 | 11:71711282-71711832 |
| 11:71800402-71801022 | 11:72240851-72241358 | 11:72243975-72244684 |
| 11:72387813-72388277 | 11:72583999-72584554 | 11:72625647-72626140 |
| 11:75429590-75430007 | 11:75606018-75606482 | 11:76322037-76322498 |
| 11:77039840-77040716 | 11:77560119-77560485 | 11:78066572-78066873 |
| 11:78961879-78962481 | 11:82732136-82732740 | 11:85665755-85666274 |
| 11:86182996-86183620 | 11:86374368-86375333 | 11:89065769-89066196 |
| 11:89786900-89787965 | 11:89863989-89865707 | 11:90051679-90053796 |
| 11:90118161-90118646 | 11:90134387-90134959 | 11:92969191-92970202 |
| 11:94400187-94401548 | 11:94706207-94706665 | 11:95521490-95523055 |
| 11:96090951-96091401 | 11:96306813-96307978 | 11:101582570-101584250 |
| 11:102324663- | 11:102653703- | 11:105070696- |
| 102325214 | 102654004 | 105071785 |
| 11:111441456- | 11:111514834- | 11:111540004- |
| 111441947 | 111515175 | 111540448 |
| 11:111631707- | 11:112120469- | 11:112355907- |
| 111632909 | 112120876 | 112356338 |
| 11:113184466- | 11:113387338- | 11:114294859- |
| 113185020 | 113388249 | 114297014 |
| 11:115941480- | 11:116580050- | 11:116707290- |
| 115941850 | 116581252 | 116707805 |
| 11:118056689- | 11:118146147- | 11:118531415- |
| 118057056 | 118146934 | 118531828 |
| 11:119585367- | 11:119742341- | 11:119994068- |
| 119585737 | 119742961 | 119996159 |
| 11:120563970- | 11:121391821- | 11:121656151- |
| 120564280 | 121392361 | 121656670 |
| 11:122099639- | 11:122614177- | 11:122714209- |
| 122100690 | 122614478 | 122716713 |
| 11:122976288- | 11:122979230- | 11:123302790- |
| 122976724 | 122979988 | 123303251 |
| 11:123358590- | 11:123559891- | 11:123598461- |
| 123358898 | 123560192 | 123598762 |
| 11:123905872- | 11:123943764- | 11:124745259- |
| 123906465 | 123944439 | 124746108 |
| 11:124868303- | 11:125496440- | 11:125904298- |
| 124869176 | 125496754 | 125904713 |
| 11:126162833- | 11:126332065- | 11:128208580- |
| 126163226 | 126332475 | 128208905 |
| 11:128549155- | 11:129143299- | 11:129379265- |
| 128549754 | 129143749 | 129380491 |
| 11:129639282- | 11:129705401- | 11:129950670- |
| 129639972 | 129705848 | 129951222 |
| 11:129960192- | 11:130449969- | 11:130847584- |
| 129960556 | 130451789 | 130848029 |
| 11:132357694- | 11:134069553- | 11:134124666- |
| 132358235 | 134069910 | 134125345 |
| 11:134330620- | 12:104571-105161 | 12:203493-204428 |
| 134331119 | | |
| 12:288764-289155 | 12:459228-459636 | 12:1540734-1542131 |
| 12:1907884-1908665 | 12:2382689-2383021 | 12:2450850-2451151 |
| 12:2752916-2753593 | 12:2793942-2794401 | 12:3170599-3171112 |
| 12:3199880-3200718 | 12:3490652-3491452 | 12:3527187-3527540 |
| 12:3552236-3552537 | 12:4006715-4007066 | 12:4142760-4144686 |
| 12:4809741-4810732 | 12:4911087-4912009 | 12:5043324-5044841 |
| 12:5433861-5434385 | 12:5556059-5556583 | 12:6182057-6182404 |
| 12:6899903-6901171 | 12:6915596-6916113 | 12:7439563-7440366 |
| 12:7872586-7873567 | 12:7971294-7972153 | 12:8374532-8375112 |
| 12:9366387-9366721 | 12:12714493-12714998 | 12:13195345-13195746 |
| 12:16604828-16607406 | 12:19191690-19192057 | 12:19382035-19382495 |
| 12:21940872-21942646 | 12:24583933-24584409 | 12:24664054-24664887 |
| 12:24740700-24742985 | 12:27627238-27627979 | 12:27667553-27667887 |
| 12:27779330-27779717 | 12:29158704-29159877 | 12:30169782-30170582 |
| 12:30200769-30201707 | 12:30753578-30754131 | 12:30931810-30932194 |
| 12:31078826-31079245 | 12:31646879-31647419 | 12:31682194-31682664 |
| 12:32252480-32253021 | 12:32448606-32448907 | 12:33438941-33440334 |
| 12:33983140-33983614 | 12:39144667-39145009 | 12:40692927-40693973 |
| 12:41277302-41277618 | 12:41743885-41744245 | 12:42039135-42039610 |
| 12:42482121-42482477 | 12:43550680-43552508 | 12:45050339-45051000 |
| 12:45051378-45051923 | 12:45065297-45065716 | 12:46468194-46468495 |
| 12:47212162-47212822 | 12:47717464-47717796 | 12:47998863-47999317 |
| 12:48184448-48185069 | 12:48528125-48528865 | 12:48717382-48717808 |
| 12:48882360-48882772 | 12:49827027-49827759 | 12:50032514-50033350 |
| 12:51324213-51324651 | 12:51394458-51395538 | 12:51868124-51869439 |
| 12:51869453-51869855 | 12:51907616-51908081 | 12:52232451-52234051 |
| 12:52250327-52250877 | 12:52600598-52601813 | 12:53218466-53220038 |
| 12:53428209-53428695 | 12:53677135-53678876 | 12:53748046-53748477 |
| 12:53927333-53927912 | 12:53938965-53939815 | 12:53954839-53955794 |
| 12:54019164-54020936 | 12:54033186-54034085 | 12:54090225-54090818 |
| 12:54217894-54218404 | 12:54369434-54370842 | 12:56267603-56268078 |
| 12:56488602-56489060 | 12:57232348-57232812 | 12:57521623-57522082 |
| 12:57576848-57577182 | 12:57829563-57831921 | 12:57852616-57853163 |
| 12:58887012-58887421 | 12:61757738-61758221 | 12:61993583-61993885 |
| 12:63003494-63003892 | 12:63149773-63150716 | 12:64104450-64104820 |
| 12:65495183-65495633 | 12:65728860-65729751 | 12:65881654-65881957 |
| 12:66188914-66189386 | 12:66646971-66647412 | 12:67534773-67535171 |
| 12:68253568-68253869 | 12:68933309-68933926 | 12:69648479-69648797 |
| 12:72823700-72825691 | 12:73129112-73129929 | 12:75207177-75208014 |
| 12:75209021-75209631 | 12:75820653-75821141 | 12:76311895-76312210 |
| 12:76455392-76455725 | 12:77062589-77063441 | 12:77571789-77572257 |
| 12:78863346-78863828 | 12:78864539-78865311 | 12:80708107-80709065 |
| 12:81955771-81956167 | 12:84912645-84913471 | 12:85279933-85281345 |
| 12:86264299-86264641 | 12:91381172-91381473 | 12:91438236-91438576 |
| 12:92603284-92604754 | 12:93109984-93110285 | 12:93302451-93302815 |
| 12:93541686-93542028 | 12:93572652-93573389 | 12:93625147-93625626 |
| 12:94761488-94761898 | 12:95547815-95548347 | 12:95548409-95549261 |
| 12:98221152-98221754 | 12:98350882-98351399 | 12:98745342-98746284 |
| 12:98893512-98895827 | 12:99154500-99155323 | 12:100714759-100716933 |
| 12:100794351- | 12:101642213- | 12:102958373- |
| 100795232 | 101642732 | 102959134 |
| 12:103113890- | 12:103672607- | 12:103859010- |
| 103115221 | 103673030 | 103859570 |
| 12:104049851- | 12:104138559- | 12:104497319- |
| 104050390 | 104139070 | 104498771 |
| 12:104667114- | 12:104792526- | 12:106580486- |
| 104667474 | 104792886 | 106581132 |
| 12:106585503- | 12:107775291- | 12:107844700- |
| 106585911 | 107776098 | 107845978 |
| 12:108066788- | 12:109154017- | 12:109654664- |
| 108067172 | 109154411 | 109654966 |
| 12:109712131- | 12:109718278- | 12:109951932- |
| 109712785 | 109718841 | 109952507 |
| 12:112575469- | 12:112792107- | 12:113471460- |
| 112575875 | 112792413 | 113471976 |
| 12:113475728- | 12:113478395- | 12:113637993- |
| 113477002 | 113479062 | 113638364 |
| 12:114407844- | 12:114414045- | 12:114440274- |
| 114408963 | 114414705 | 114441626 |
| 12:114447332- | 12:114448454- | 12:114682748- |
| 114447700 | 114448888 | 114683295 |
| 12:114692121- | 12:114697971- | 12:114915968- |
| 114693254 | 114698695 | 114917085 |
| 12:115167312- | 12:116370038- | 12:116497061- |
| 115167750 | 116370483 | 116497465 |
| 12:116999188- | 12:117360559- | 12:118774256- |
| 117000553 | 117361465 | 118774773 |
| 12:118980638- | 12:119335073- | 12:119828327- |
| 118981544 | 119335385 | 119828628 |
| 12:120182310- | 12:120361529- | 12:120536348- |
| 120182674 | 120362358 | 120536774 |
| 12:121189609- | 12:121578226- | 12:121812981- |
| 121190745 | 121579000 | 121813411 |
| 12:121918793- | 12:121923827- | 12:121960702- |
| 121919097 | 121924189 | 121961003 |
| 12:122020742- | 12:122850700- | 12:123272374- |
| 122021269 | 122851139 | 123273164 |
| 12:123670657- | 12:123916785- | 12:124096789- |
| 123670964 | 123917100 | 124097269 |
| 12:124294249- | 12:124517396- | 12:124551634- |
| 124294970 | 124519227 | 124552015 |
| 12:124676441- | 12:124803875- | 12:124824722- |
| 124676759 | 124804316 | 124825023 |
| 12:125263150- | 12:125683744- | 12:126190866- |
| 125263635 | 125684047 | 126191909 |
| 12:126726511- | 12:126727464- | 12:127455788- |
| 126727235 | 126728183 | 127456564 |
| 12:128268080- | 12:128645216- | 12:128821926- |
| 128268674 | 128645539 | 128822766 |
| 12:129036171- | 12:129903201- | 12:130042393- |
| 129036472 | 129904690 | 130042891 |
| 12:130163011- | 12:130338911- | 12:130468465- |
| 130164263 | 130339706 | 130469488 |
| 12:130517979- | 12:130670384- | 12:130700180- |
| 130518524 | 130671552 | 130700481 |
| 12:130704558- | 12:130796938- | 12:130999748- |
| 130705186 | 130797396 | 131001126 |
| 12:131019406- | 12:131091250- | 12:131661195- |
| 131019942 | 131091694 | 131661716 |
| 12:131710228- | 12:131825353- | 12:131865206- |
| 131710641 | 131825782 | 131865699 |
| 12:132264505- | 12:132282679- | 12:132323226- |
| 132264806 | 132283318 | 132324135 |
| 12:132439392- | 12:132618378- | 12:132803710- |
| 132440672 | 132620120 | 132804147 |
| 12:132904552-132905122 | 13:19344662-19345230 | 13:19564472-19565039 |
| 13:20301282-20302020 | 13:20946041-20946480 | 13:21668528-21669054 |
| 13:22837920-22838739 | 13:23751124-23751562 | 13:24216286-24216621 |
| 13:24328172-24328708 | 13:24745898-24746503 | 13:25170132-25171173 |
| 13:25171477-25172001 | 13:25372430-25372733 | 13:25469003-25469554 |
| 13:26050380-26050961 | 13:26300194-26300495 | 13:27131159-27132228 |
| 13:27343712-27344013 | 13:27791294-27791867 | 13:27791610-27791929 |
| 13:27792889-27795588 | 13:27928097-27929549 | 13:27965992-27966601 |
| 13:27969221-27971621 | 13:27979489-27980374 | 13:28538745-28541059 |
| 13:29846844-29848014 | 13:30569853-30570424 | 13:30905906-30906756 |
| 13:33349575-33350117 | 13:33968787-33969147 | 13:35470481-35471428 |
| 13:36155016-36155392 | 13:37403635-37404133 | 13:40613375-40613940 |
| 13:41131734-41132317 | 13:41456668-41457112 | 13:44350773-44351529 |
| 13:44373143-44373674 | 13:44755814-44756162 | 13:44815801-44816221 |
| 13:45311640-45312200 | 13:47904307-47904712 | 13:48265104-48265509 |
| 13:49220486-49221629 | 13:50843497-50844163 | 13:52208307-52208608 |
| 13:52739004-52739316 | 13:52739641-52740242 | 13:52846535-52848796 |
| 13:52849570-52850108 | 13:53151864-53152171 | 13:53200558-53202360 |
| 13:57632296-57634741 | 13:62626151-62626725 | 13:65310881-65311300 |
| 13:71864339-71864822 | 13:75635124-75635680 | 13:77917884-77918762 |
| 13:78586797-78587190 | 13:78595358-78597695 | 13:78601244-78602016 |
| 13:78605215-78605532 | 13:78607122-78608237 | 13:83879493-83880022 |
| 13:83907227-83907528 | 13:92340570-92341129 | 13:93847196-93847598 |
| 13:94701819-94702757 | 13:94705187-94705696 | 13:95552017-95552849 |
| 13:97328465-97328766 | 13:98978059-98978668 | 13:99311250-99314757 |
| 13:99314875-99315176 | 13:99859865-99860258 | 13:99895309-99896592 |
| 13:99958676-99959224 | 13:99971910-99972414 | 13:99988978-99989502 |
| 13:99996920-99997879 | 13:100128865-100130057 | 13:100566668-100567110 |
| 13:101208940- | 13:102803784- | 13:102825901- |
| 101209266 | 102804216 | 102826248 |
| 13:106155233- | 13:106533985- | 13:107865679- |
| 106155794 | 106534410 | 107867410 |
| 13:107867587- | 13:108213893- | 13:108494744- |
| 107868991 | 108215031 | 108496933 |
| 13:108665560- | 13:109140217- | 13:109519512- |
| 108666083 | 109141454 | 109519813 |
| 13:109570307- | 13:109727758- | 13:109783993- |
| 109570627 | 109728265 | 109784743 |
| 13:110006369- | 13:110343114- | 13:110676663- |
| 110006748 | 110343571 | 110678340 |
| 13:110812243- | 13:112053735- | 13:112062160- |
| 110813427 | 112057751 | 112063089 |
| 13:112065673- | 13:112067478- | 13:112105039- |
| 112066081 | 112069385 | 112106150 |
| 13:112333448- | 13:112693496- | 13:112730058- |
| 112334543 | 112694228 | 112730727 |
| 13:112749004- | 13:112752420- | 13:112782143- |
| 112749471 | 112753271 | 112782514 |
| 13:112837527- | 13:112841892- | 13:112879367- |
| 112838691 | 112842237 | 112879810 |
| 13:112943131- | 13:112968320- | 13:112993977- |
| 112944188 | 112969525 | 112994604 |
| 13:113001653- | 13:113008721- | 13:113109289- |
| 113002198 | 113009366 | 113110365 |
| 13:113110867- | 13:113152826- | 13:113187223- |
| 113111427 | 113153767 | 113187791 |
| 13:113313954- | 13:113364500- | 13:113832032- |
| 113315452 | 113365284 | 113832616 |
| 13:113862549- | 13:114068961- | 13:114140858- |
| 113863050 | 114069515 | 114141159 |
| 13:114295079-114295748 | 14:21022896-21024355 | 14:21092663-21092964 |
| 14:21992873-21993174 | 14:23183739-23184130 | 14:23419092-23420460 |
| 14:24171589-24172966 | 14:26204828-26205158 | 14:28766507-28767015 |
| 14:28767847-28768764 | 14:28774049-28774819 | 14:28785011-28785765 |
| 14:30875423-30876029 | 14:30966394-30967770 | 14:34035630-34035995 |
| 14:34043486-34043888 | 14:34053907-34054368 | 14:35375613-35375946 |
| 14:36503862-36504740 | 14:36516963-36518456 | 14:36521464-36522131 |
| 14:36522775-36523211 | 14:36579835-36580839 | 14:36583809-36584655 |
| 14:36662361-36663611 | 14:36666317-36667229 | 14:37584618-37585130 |
| 14:37585924-37586231 | 14:37591950-37592850 | 14:37610885-37611623 |
| 14:37622091-37623001 | 14:38209615-38211514 | 14:38254953-38256078 |
| 14:41607894-41608391 | 14:46881957-46882706 | 14:47674143-47676382 |
| 14:50121712-50122034 | 14:50823268-50823602 | 14:50847945-50848288 |
| 14:51077602-51078136 | 14:52951945-52952544 | 14:54211683-54212052 |
| 14:56119642-56120757 | 14:56794264-56795259 | 14:56808114-56810272 |
| 14:56811269-56812279 | 14:56817008-56817652 | 14:57051798-57052099 |
| 14:57837448-57838148 | 14:58639779-58640095 | 14:60507007-60507795 |
| 14:60510307-60510908 | 14:60515212-60516273 | 14:60637152-60638091 |
| 14:61279867-61282007 | 14:61327397-61328675 | 14:61332541-61333110 |
| 14:61574496-61574907 | 14:64541126-64543010 | 14:64716664-64716981 |
| 14:68703113-68703612 | 14:69428547-69428923 | 14:69483554-69483959 |
| 14:69547612-69548077 | 14:70692122-70692625 | 14:74014521-74014996 |
| 14:75875925-75879307 | 14:76138636-76139046 | 14:76742836-76743505 |
| 14:77459342-77459903 | 14:77565300-77566111 | 14:77738793-77739255 |
| 14:85530295-85533672 | 14:85534511-85534918 | 14:87573248-87573753 |
| 14:88322429-88322934 | 14:89160352-89160867 | 14:89378207-89378599 |
| 14:91119896-91120362 | 14:92026495-92027119 | 14:92625246-92625646 |
| 14:92645956-92646410 | 14:92922822-92923149 | 14:93165797-93166144 |
| 14:93938741-93939527 | 14:94581331-94581655 | 14:94768141-94768711 |
| 14:94772934-94774381 | 14:95253297-95253598 | 14:95426558-95427598 |
| 14:95516861-95517951 | 14:95875758-95876179 | 14:97032998-97033744 |
| 14:99118067-99118823 | 14:99248629-99249719 | 14:99347599-99348270 |
| 14:99427652-99428058 | 14:99756843-99757735 | 14:99767060-99767361 |
| 14:100341128- | 14:100656838- | 14:100708769- |
| 100341460 | 100657573 | 100709812 |
| 14:100884606- | 14:100893660- | 14:100905749- |
| 100884990 | 100894961 | 100906050 |
| 14:100938574- | 14:100988357- | 14:100991936- |
| 100940629 | 100989329 | 100992394 |
| 14:101047067- | 14:101248038- | 14:101409117- |
| 101049669 | 101249007 | 101409459 |
| 14:101559356- | 14:101561195- | 14:101562506- |
| 101560108 | 101562122 | 101563340 |
| 14:101781115- | 14:101790198- | 14:102508230- |
| 101781791 | 101790652 | 102509284 |
| 14:102930438- | 14:102946391- | 14:103011156- |
| 102930883 | 102946918 | 103011609 |
| 14:103102068- | 14:103138664- | 14:103216449- |
| 103102951 | 103138970 | 103216959 |
| 14:103361748- | 14:103557963- | 14:103980796- |
| 103362338 | 103558319 | 103981098 |
| 14:104117345- | 14:104348794- | 14:104687952- |
| 104118175 | 104349179 | 104689341 |
| 14:104801968- | 14:104866114- | 14:104896470- |
| 104803108 | 104866511 | 104896800 |
| 14:104897269- | 14:105248951- | 14:105679640- |
| 104897640 | 105249829 | 105679941 |
| 15:22714245-22715786 | 15:22775912-22776762 | 15:22878202-22878533 |
| 15:23179729-23180441 | 15:23226016-23226506 | 15:23447046-23447593 |
| 15:26868072-26868867 | 15:26967607-26968399 | 15:26971341-26971757 |
| 15:28532869-28533227 | 15:28788506-28789034 | 15:29106921-29107222 |
| 15:29329285-29329619 | 15:31325017-31325655 | 15:32312603-32313448 |
| 15:32687535-32687994 | 15:33013818-33014210 | 15:33649736-33650267 |
| 15:34239367-34239776 | 15:34494434-34496056 | 15:34753345-34754777 |
| 15:36470816-36471553 | 15:36880301-36880720 | 15:37095101-37097577 |
| 15:38565098-38565408 | 15:40252010-40252540 | 15:40279767-40281989 |
| 15:40283414-40284114 | 15:40952433-40952888 | 15:41502063-41503342 |
| 15:41512238-41513249 | 15:42737451-42737797 | 15:43793565-43794115 |
| 15:45111648-45112873 | 15:45704107-45704788 | 15:47184024-47184785 |
| 15:48191413-48192049 | 15:51215026-51216196 | 15:52252789-52253159 |
| 15:52505165-52505840 | 15:52783073-52783466 | 15:52788007-52789230 |
| 15:52794283-52795435 | 15:55588837-55589395 | 15:58064972-58065812 |
| 15:58438904-58439400 | 15:58866080-58866648 | 15:59439687-59440036 |
| 15:60004266-60005491 | 15:62403809-62404159 | 15:62952883-62953481 |
| 15:63060758-63061227 | 15:64700431-64701283 | 15:64824035-64824627 |
| 15:64840705-64841183 | 15:65076971-65078067 | 15:65301882-65302545 |
| 15:65666252-65666602 | 15:66621585-66622344 | 15:67003630-67004106 |
| 15:67783289-67783656 | 15:67825846-67826941 | 15:67828473-67829517 |
| 15:68812644-68813110 | 15:69043498-69043947 | 15:69358383-69358689 |
| 15:70825835-70826136 | 15:71800120-71800513 | 15:72086500-72086801 |
| 15:72195317-72195702 | 15:72675796-72676097 | 15:73346719-73347020 |
| 15:74072611-74073076 | 15:74132838-74133834 | 15:74955623-74956306 |
| 15:75037690-75038082 | 15:75346638-75347243 | 15:75687689-75688247 |
| 15:76335074-76335647 | 15:76936881-76937185 | 15:78338785-78339086 |
| 15:78641274-78641671 | 15:78872222-78872559 | 15:79089230-79089886 |
| 15:79282653-79282982 | 15:79432837-79433558 | 15:79768959-79769360 |
| 15:79849458-79849981 | 15:79900122-79900426 | 15:81134211-81134663 |
| 15:82298408-82298951 | 15:83283691-83285218 | 15:84079405-84079916 |
| 15:84976855-84977344 | 15:85282564-85283126 | 15:85408296-85408732 |
| 15:85810083-85810442 | 15:88603842-88604343 | 15:88769577-88770090 |
| 15:88802948-88803757 | 15:88921086-88921458 | 15:89360229-89360639 |
| 15:89366368-89367591 | 15:89377414-89378927 | 15:89406076-89406547 |
| 15:89820762-89822563 | 15:89936604-89936912 | 15:90000011-90000674 |
| 15:90414669-90415140 | 15:91129847-91130315 | 15:91456523-91457069 |
| 15:95326578-95327397 | 15:96296280-96296741 | 15:98101843-98103323 |
| 15:98414110-98414657 | 15:99019592-99020069 | 15:99403519-99403820 |
| 15:99434880-99435437 | 15:99452817-99453522 | 15:100713194-100714025 |
| 15:100880242-100880935 | 15:100973858-100974351 | 15:101199604-101200315 |
| 16:94262-94563 | 16:165787-166396 | 16:176971-177495 |
| 16:180666-180969 | 16:369712-370259 | 16:476210-477726 |
| 16:535236-535650 | 16:621721-622759 | 16:780443-781196 |
| 16:945965-946435 | 16:1065629-1066166 | 16:1154115-1155169 |
| 16:1978847-1980019 | 16:2023604-2023942 | 16:2124887-2125330 |
| 16:2178485-2179165 | 16:2967358-2968305 | 16:3017310-3018863 |
| 16:3160174-3160665 | 16:4114889-4115413 | 16:4609255-4609678 |
| 16:4660815-4662215 | 16:4987552-4988480 | 16:5012298-5012599 |
| 16:6018765-6019881 | 16:6023358-6023820 | 16:6248658-6249154 |
| 16:6372049-6374163 | 16:6482891-6484356 | 16:7304305-7304873 |
| 16:8969527-8969828 | 16:9090122-9090458 | 16:9763120-9763986 |
| 16:10180091-10180623 | 16:10183239-10183803 | 16:10341941-10342302 |
| 16:10818177-10819052 | 16:10868559-10869105 | 16:10990361-10990662 |
| 16:11183223-11183631 | 16:11303346-11303791 | 16:11629054-11630579 |
| 16:12089914-12090569 | 16:12147451-12147848 | 16:12406829-12407228 |
| 16:13550280-13550868 | 16:14405749-14406125 | 16:15033073-15034134 |
| 16:15144098-15144485 | 16:15501981-15502535 | 16:16009589-16009890 |
| 16:17238569-17238944 | 16:17348414-17348876 | 16:17557453-17557754 |
| 16:17932748-17933228 | 16:19474004-19474399 | 16:20073429-20074015 |
| 16:20348406-20349326 | 16:21167314-21168408 | 16:22199530-22200034 |
| 16:22812988-22814120 | 16:22814704-22815310 | 16:22947981-22948771 |
| 16:23124243-23124766 | 16:23712771-23713731 | 16:23835247-23835723 |
| 16:23836146-23836672 | 16:24255513-24256563 | 16:24850404-24851797 |
| 16:25692487-25693279 | 16:26951965-26952347 | 16:27048055-27048587 |
| 16:27214286-27215000 | 16:27253456-27253933 | 16:27355674-27356082 |
| 16:28310203-28311040 | 16:28596716-28597396 | 16:29082319-29082750 |
| 16:29141894-29142556 | 16:29289064-29289578 | 16:29785205-29785796 |
| 16:30011930-30012727 | 16:30069072-30069580 | 16:30332687-30333258 |
| 16:30567136-30568717 | 16:30947773-30948362 | 16:31045749-31046130 |
| 16:31118589-31118968 | 16:31214532-31214994 | 16:31471339-31473552 |
| 16:31488378-31489222 | 16:31568295-31569852 | 16:31819160-31820017 |
| 16:32084037-32086123 | 16:32202470-32202978 | 16:47057580-47058849 |
| 16:48810028-48811101 | 16:49275064-49275592 | 16:49277676-49278247 |
| 16:49280015-49283579 | 16:49876113-49876504 | 16:50549259-50549704 |
| 16:50841004-50841664 | 16:53770653-53771084 | 16:53817060-53817542 |
| 16:54288287-54291751 | 16:54342176-54342540 | 16:54932406-54935166 |
| 16:54936258-54938019 | 16:55056479-55057318 | 16:55565860-55567447 |
| 16:55656385-55657203 | 16:55944416-55944966 | 16:56275060-56275510 |
| 16:56286761-56287191 | 16:56605995-56606361 | 16:57802752-57803691 |
| 16:57916129-57917450 | 16:61743455-61744350 | 16:62035283-62037253 |
| 16:66513815-66514233 | 16:66844419-66845009 | 16:66935336-66936071 |
| 16:66961810-66962276 | 16:67000257-67000784 | 16:67001160-67001584 |
| 16:67149537-67150185 | 16:67189081-67190652 | 16:67416639-67417065 |
| 16:68286453-68287763 | 16:68446887-68447911 | 16:68509947-68510316 |
| 16:68992456-68992947 | 16:70392160-70392516 | 16:70686304-70686764 |
| 16:71460123-71460486 | 16:72924472-72925190 | 16:73386711-73387301 |
| 16:74833595-74833896 | 16:75273463-75273883 | 16:75494397-75494859 |
| 16:75601861-75602284 | 16:76428948-76429418 | 16:77434478-77435351 |
| 16:77585237-77585792 | 16:77861961-77862487 | 16:77877751-77878137 |
| 16:77912801-77913138 | 16:79170704-79171972 | 16:79640301-79641186 |
| 16:79658265-79658745 | 16:81431822-81433574 | 16:81530321-81530879 |
| 16:81583080-81584536 | 16:81700408-81700807 | 16:83344750-83345051 |
| 16:83903516-83903993 | 16:83969057-83969456 | 16:84405140-84405441 |
| 16:84553929-84554385 | 16:84735720-84736260 | 16:85098555-85099755 |
| 16:85239999-85240300 | 16:85477824-85478201 | 16:85915193-85915618 |
| 16:86287031-86287648 | 16:86494757-86495490 | 16:86507676-86508562 |
| 16:86510816-86511538 | 16:86515339-86516296 | 16:86565004-86566055 |
| 16:86578035-86579761 | 16:87196311-87196978 | 16:87414066-87414752 |
| 16:87539967-87541888 | 16:87860308-87861231 | 16:87863535-87864022 |
| 16:87865955-87868653 | 16:87897511-87898028 | 16:87936687-87937223 |
| 16:88100441-88101392 | 16:88128847-88130548 | 16:88189309-88190423 |
| 16:88234057-88234911 | 16:88240043-88240366 | 16:88259217-88259527 |
| 16:88436565-88436889 | 16:88437202-88437920 | 16:88469660-88471003 |
| 16:88591558-88592186 | 16:88838902-88839534 | 16:88982604-88984632 |
| 16:89161247-89161548 | 16:89191516-89191828 | 16:89202272-89202713 |
| 16:89233565-89233992 | 16:89247985-89248863 | 16:89315408-89317296 |
| 16:89359189-89359743 | 16:89576356-89576813 | 16:89581044-89581612 |
| 17:162115-162416 | 17:257908-258327 | 17:304503-305547 |
| 17:868069-868385 | 17:1095025-1095475 | 17:1498925-1499329 |
| 17:1747093-1747619 | 17:1771710-1772234 | 17:1907723-1908148 |
| 17:2057472-2058557 | 17:2137327-2137708 | 17:2240112-2240570 |
| 17:2723539-2724026 | 17:2941948-2942249 | 17:3385984-3386851 |
| 17:3600556-3601000 | 17:3755161-3755659 | 17:4229630-4229994 |
| 17:4276585-4276886 | 17:4594779-4595301 | 17:4624824-4625498 |
| 17:4785125-4786749 | 17:4787927-4788417 | 17:5097006-5098175 |
| 17:5115529-5116291 | 17:5122457-5122763 | 17:5694541-5694985 |
| 17:6223473-6223789 | 17:6507571-6507985 | 17:6608356-6608732 |
| 17:6684887-6685214 | 17:6712842-6713344 | 17:6713480-6714429 |
| 17:7022883-7023645 | 17:7043994-7044434 | 17:7174069-7174739 |
| 17:7477495-7478730 | 17:7588912-7589398 | 17:8003008-8004088 |
| 17:8015631-8016119 | 17:8327357-8327773 | 17:8408090-8408421 |
| 17:8921195-8923221 | 17:8951411-8953652 | 17:8965432-8966121 |
| 17:9022458-9023538 | 17:9959520-9960082 | 17:10000328-10000859 |
| 17:10022393-10022742 | 17:10196037-10196565 | 17:10198927-10199457 |
| 17:10248157-10248459 | 17:11241531-11242249 | 17:11871954-11872256 |
| 17:13600364-13600806 | 17:14297590-14297897 | 17:14297716-14298817 |
| 17:15409609-15409947 | 17:15782756-15783310 | 17:15917734-15918205 |
| 17:15920694-15921124 | 17:16666475-16667420 | 17:16683512-16684086 |
| 17:17495639-17495942 | 17:17545439-17545740 | 17:17819070-17820257 |
| 17:18181352-18181866 | 17:18376343-18377958 | 17:19004861-19005265 |
| 17:19469301-19469718 | 17:20028513-20028814 | 17:20234511-20235044 |
| 17:20416411-20416940 | 17:20840902-20841445 | 17:20843336-20843968 |
| 17:21044151-21044634 | 17:21316934-21317396 | 17:21419004-21419520 |
| 17:21452584-21453416 | 17:27551969-27552414 | 17:27608250-27608721 |
| 17:27609103-27609620 | 17:27984759-27985060 | 17:28229409-28230832 |
| 17:28232912-28235300 | 17:28436601-28437026 | 17:29006286-29006902 |
| 17:29612446-29613224 | 17:30953065-30954677 | 17:32135326-32135889 |
| 17:32161714-32162193 | 17:32240825-32241126 | 17:32496657-32497112 |
| 17:32795670-32796059 | 17:34037792-34038093 | 17:34156259-34157429 |
| 17:34625996-34626839 | 17:35449530-35450516 | 17:35489956-35490883 |
| 17:35496417-35496929 | 17:35497722-35498531 | 17:35740839-35741385 |
| 17:35752751-35753439 | 17:35756189-35756652 | 17:36182330-36183009 |
| 17:36464585-36465054 | 17:36482448-36482750 | 17:36808067-36808696 |
| 17:36939548-36943529 | 17:38815711-38816072 | 17:39605935-39606386 |
| 17:39627190-39627647 | 17:40323150-40326934 | 17:40664989-40665386 |
| 17:40779438-40779958 | 17:41907703-41908175 | 17:42653683-42654186 |
| 17:42680114-42680584 | 17:43953277-43954040 | 17:44086836-44087143 |
| 17:44235519-44236773 | 17:44655933-44657475 | 17:44960029-44960591 |
| 17:45305702-45308048 | 17:45413296-45414022 | 17:45429297-45430909 |
| 17:45896732-45897946 | 17:46266117-46266523 | 17:46769676-46770167 |
| 17:46850695-46851098 | 17:47423976-47424491 | 17:48527243-48527692 |
| 17:48553902-48557335 | 17:48564456-48564873 | 17:48568474-48568797 |
| 17:48589369-48590738 | 17:48591931-48592289 | 17:48595595-48596408 |
| 17:48615022-48616143 | 17:48718687-48719883 | 17:48747316-48748179 |
| 17:48749980-48750530 | 17:48754646-48755546 | 17:48995259-48996152 |
| 17:49694895-49695368 | 17:50468060-50469486 | 17:50478177-50478661 |
| 17:51165147-51165869 | 17:52157884-52160238 | 17:55162122-55162563 |
| 17:55264055-55264459 | 17:55458970-55459398 | 17:55845482-55845886 |
| 17:56834654-56835728 | 17:57045184-57045965 | 17:57406308-57406820 |
| 17:57761767-57762231 | 17:58157183-58157728 | 17:59940728-59941043 |
| 17:60082520-60082913 | 17:60141117-60141519 | 17:60161919-60162239 |
| 17:61395432-61396069 | 17:61397620-61398361 | 17:61401069-61405644 |
| 17:61454101-61455043 | 17:61456727-61457637 | 17:62137705-62138750 |
| 17:63537883-63538824 | 17:63838627-63839436 | 17:63849235-63849868 |
| 17:64196718-64197063 | 17:64732890-64733191 | 17:64778316-64779818 |
| 17:65103950-65104294 | 17:65463110-65463468 | 17:67608640-67608975 |
| 17:68198264-68199341 | 17:68305110-68305564 | 17:68312258-68313236 |
| 17:71071761-71072197 | 17:72116502-72118139 | 17:72632027-72632353 |
| 17:72640586-72641113 | 17:72702306-72702905 | 17:72953682-72953983 |
| 17:73478566-73478911 | 17:73560718-73561645 | 17:73598649-73598950 |
| 17:73711099-73711522 | 17:73901683-73902873 | 17:73949876-73951811 |
| 17:74042553-74043049 | 17:74051284-74051646 | 17:74215353-74215819 |
| 17:74403860-74404393 | 17:74670774-74671839 | 17:74935325-74935981 |
| 17:74971870-74972291 | 17:75088084-75088550 | 17:75587608-75588364 |
| 17:75634148-75634613 | 17:75694547-75696411 | 17:76075976-76076971 |
| 17:76076877-76078035 | 17:76291912-76292512 | 17:76483354-76486014 |
| 17:76496492-76497412 | 17:76711334-76711655 | 17:76961904-76962278 |
| 17:77273591-77274013 | 17:77412760-77413229 | 17:77451222-77451827 |
| 17:77489720-77490142 | 17:77802870-77803383 | 17:77871372-77872662 |
| 17:77959908-77960426 | 17:78142284-78142700 | 17:78158555-78158908 |
| 17:78376056-78376405 | 17:78532845-78533350 | 17:78599388-78600838 |
| 17:78645864-78647511 | 17:78779874-78780643 | 17:78903487-78904947 |
| 17:79038455-79039239 | 17:79097560-79097873 | 17:79427623-79428158 |
| 17:79941999-79943219 | 17:80220899-80221824 | 17:80276948-80277249 |
| 17:80363491-80363906 | 17:80411156-80411462 | 17:80677489-80679376 |
| 17:80726821-80727630 | 17:80811520-80812187 | 17:80826751-80827487 |
| 17:80859020-80859321 | 17:80984828-80985221 | 17:81154823-81155495 |
| 17:81341378-81342753 | 17:81397796-81398896 | 17:81518491-81518872 |
| 17:81647709-81648710 | 17:81936646-81936972 | 17:81961635-81962805 |
| 17:82003496-82003981 | 17:82050710-82051235 | 17:82334053-82334356 |
| 17:82371977-82372830 | 17:83070952-83071360 | 17:83081733-83082146 |
| 18:72914-73597 | 18:904169-904799 | 18:906602-907698 |
| 18:908743-909723 | 18:3045262-3045587 | 18:4248536-4248914 |
| 18:4453726-4455761 | 18:5196128-5196728 | 18:5247705-5248032 |
| 18:8608508-8608980 | 18:8891330-8891631 | 18:9743410-9744127 |
| 18:10032606-10033472 | 18:10453937-10454309 | 18:10950698-10950999 |
| 18:11033860-11035231 | 18:11751265-11752259 | 18:11871320-11871879 |
| 18:11872643-11873173 | 18:12254485-12255331 | 18:12256827-12257150 |
| 18:12287167-12287519 | 18:12569329-12569760 | 18:12911463-12912625 |
| 18:13288504-13289025 | 18:13450816-13452972 | 18:13564504-13564805 |
| 18:13610967-13611728 | 18:13641325-13642353 | 18:14449982-14450287 |
| 18:14747287-14748309 | 18:15197384-15198359 | 18:21740053-21740354 |
| 18:22166220-22167839 | 18:22172057-22173398 | 18:23822938-23823299 |
| 18:26546668-26547833 | 18:30175780-30177427 | 18:31041418-31042469 |
| 18:32386510-32386857 | 18:34223502-34223947 | 18:36186341-36186700 |
| 18:37274136-37275756 | 18:37484841-37485409 | 18:38941113-38941635 |
| 18:45212622-45212923 | 18:45837274-45837727 | 18:46072513-46072824 |
| 18:46098597-46098905 | 18:46632721-46633083 | 18:47248547-47249351 |
| 18:47251250-47251753 | 18:47260918-47261522 | 18:47263568-47264621 |
| 18:48629964-48630400 | 18:48749683-48750032 | 18:48997599-48998438 |
| 18:49031467-49032213 | 18:55830521-55831023 | 18:55833739-55834111 |
| 18:55955052-55955627 | 18:56248572-56248950 | 18:57072543-57073946 |
| 18:57121589-57122241 | 18:57352412-57354660 | 18:57427994-57428713 |
| 18:57436143-57436826 | 18:57438312-57440616 | 18:57729054-57729393 |
| 18:58361437-58362743 | 18:58520799-58522592 | 18:58579527-58580102 |
| 18:58755278-58755754 | 18:59264278-59265346 | 18:59272383-59273266 |
| 18:59696338-59696648 | 18:59814936-59815339 | 18:59874124-59874600 |
| 18:63149296-63149856 | 18:63301233-63301745 | 18:63443757-63445111 |
| 18:63661843-63663749 | 18:65749717-65751366 | 18:67070900-67071430 |
| 18:67418169-67418522 | 18:72541647-72542137 | 18:72866613-72867424 |
| 18:72868005-72869786 | 18:73757747-73758108 | 18:74040876-74041497 |
| 18:74520077-74520392 | 18:75067042-75067376 | 18:75203831-75204515 |
| 18:75455299-75455701 | 18:75916012-75916853 | 18:76563994-76564575 |
| 18:76683176-76683477 | 18:76968091-76968457 | 18:76989168-76989612 |
| 18:77069730-77070444 | 18:77209027-77209944 | 18:77250392-77252002 |
| 18:77392920-77393221 | 18:77690072-77691010 | 18:77691174-77691475 |
| 18:78390614-78391470 | 18:78504233-78505251 | 18:78515331-78515632 |
| 18:78640919-78643360 | 18:78644240-78647670 | 18:78718928-78719360 |
| 18:78726405-78726875 | 18:78726612-78726940 | 18:78729353-78729654 |
| 18:78973146-78974104 | 18:78976558-78978966 | 18:79338907-79339589 |
| 18:79378897-79379198 | 18:79637465-79638370 | 18:79714702-79715061 |
| 18:79788432-79789111 | 18:80010580-80012087 | 19:339486-339922 |
| 19:345112-345575 | 19:709788-711216 | 19:719405-719915 |
| 19:752562-753224 | 19:853174-853622 | 19:958727-959256 |
| 19:1046102-1047500 | 19:1081507-1083032 | 19:1287269-1287984 |
| 19:1295476-1295935 | 19:1406323-1406794 | 19:1503073-1503380 |
| 19:1522768-1525559 | 19:1775063-1775651 | 19:1789290-1789745 |
| 19:2092889-2095878 | 19:2243968-2244504 | 19:2574294-2574700 |
| 19:2691967-2692377 | 19:3398721-3399158 | 19:3552604-3553303 |
| 19:3708057-3708379 | 19:3868508-3869029 | 19:3883424-3883725 |
| 19:4042540-4043469 | 19:4050627-4053480 | 19:4084045-4084362 |
| 19:4719672-4720151 | 19:4908886-4909332 | 19:5048606-5049026 |
| 19:5567955-5568447 | 19:6463688-6464187 | 19:6519093-6519469 |
| 19:7135402-7135892 | 19:7533308-7533774 | 19:7550831-7551289 |
| 19:7682087-7682491 | 19:7730605-7732296 | 19:7892510-7893734 |
| 19:7986371-7987393 | 19:8332973-8334125 | 19:8423293-8424473 |
| 19:8539868-8541521 | 19:8698208-8698526 | 19:9363273-9363714 |
| 19:9406664-9407246 | 19:9497921-9498872 | 19:10095714-10097024 |
| 19:10381036-10381404 | 19:10479198-10479643 | 19:10514712-10515364 |
| 19:10586891-10587457 | 19:10755082-10755544 | 19:11156069-11156510 |
| 19:11479351-11479735 | 19:11480268-11481357 | 19:12034983-12035403 |
| 19:12155477-12155868 | 19:12194589-12195085 | 19:12513405-12514123 |
| 19:12774739-12775163 | 19:12799720-12800104 | 19:13010296-13011168 |
| 19:13024710-13025630 | 19:13814492-13814838 | 19:13820576-13821156 |
| 19:14057192-14057668 | 19:14209341-14210098 | 19:14380115-14381342 |
| 19:14676095-14676397 | 19:14979227-14979685 | 19:15010238-15011607 |
| 19:15199470-15199909 | 19:15231622-15232606 | 19:15288687-15289084 |
| 19:15514969-15516417 | 19:16283491-16284001 | 19:16341890-16342608 |
| 19:16585738-16586398 | 19:16923924-16924343 | 19:16975812-16976364 |
| 19:17006803-17008417 | 19:17077795-17078187 | 19:17455518-17455913 |
| 19:17766432-17767188 | 19:17831113-17831908 | 19:17840932-17842532 |
| 19:17948801-17949489 | 19:18009553-18010102 | 19:18453285-18453684 |
| 19:18649437-18651267 | 19:18700749-18701395 | 19:18785557-18785940 |
| 19:18787405-18789019 | 19:18790998-18791649 | 19:18843277-18844302 |
| 19:19097290-19098519 | 19:19539808-19540432 | 19:19784388-19784689 |
| 19:19860620-19861203 | 19:22215234-22215628 | 19:22286053-22286979 |
| 19:22422034-22422664 | 19:22427448-22427751 | 19:22428185-22428555 |
| 19:22531567-22532945 | 19:22623210-22624046 | 19:22633923-22634968 |
| 19:22783358-22784112 | 19:22807057-22807921 | 19:23002611-23003604 |
| 19:23070251-23071685 | 19:23075141-23075866 | 19:23203699-23204417 |
| 19:23470741-23471524 | 19:23957581-23958057 | 19:23971604-23972163 |
| 19:27746935-27747381 | 19:28405683-28406135 | 19:28793357-28794244 |
| 19:29013294-29013730 | 19:29524750-29527335 | 19:29529485-29530340 |
| 19:29538169-29538483 | 19:30123397-30123698 | 19:30226658-30226961 |
| 19:30374827-30375716 | 19:31140059-31140564 | 19:31232710-31233079 |
| 19:32253535-32253836 | 19:32675914-32676226 | 19:32676508-32676963 |
| 19:32824271-32825353 | 19:33300985-33301494 | 19:33303691-33305656 |
| 19:33620997-33621706 | 19:33622840-33623597 | 19:34577128-34578088 |
| 19:34904885-34905838 | 19:34990479-34991121 | 19:35319941-35321597 |
| 19:35431222-35431589 | 19:35533201-35533897 | 19:35557386-35558893 |
| 19:35755279-35757475 | 19:35783512-35783880 | 19:35797983-35798308 |
| 19:35959493-35960369 | 19:35999226-35999734 | 19:36113782-36114175 |
| 19:36244929-36246520 | 19:36421210-36422274 | 19:36791349-36792355 |
| 19:36797264-36797971 | 19:37550861-37551927 | 19:37691744-37692710 |
| 19:37817172-37818017 | 19:37854930-37855670 | 19:38061367-38061726 |
| 19:38255690-38256212 | 19:38394478-38396178 | 19:38418278-38418745 |
| 19:38949756-38950496 | 19:39203877-39204397 | 19:39247034-39248889 |
| 19:39248776-39249271 | 19:39264392-39265915 | 19:39266327-39267595 |
| 19:39502595-39502906 | 19:39524503-39524886 | 19:40217092-40217524 |
| 19:40394780-40395082 | 19:40848234-40848872 | 19:40880198-40880737 |
| 19:41025728-41026194 | 19:41090127-41090707 | 19:41135737-41136287 |
| 19:41225488-41225929 | 19:42252247-42252702 | 19:42323298-42324083 |
| 19:42888951-42890053 | 19:42926195-42926674 | 19:43391165-43391514 |
| 19:43465413-43465902 | 19:43699258-43699985 | 19:43820143-43820950 |
| 19:43826904-43827238 | 19:44051086-44051408 | 19:44111814-44112462 |
| 19:44648989-44649317 | 19:44943564-44944096 | 19:45069055-45069383 |
| 19:45101888-45102258 | 19:45151984-45152945 | 19:45472337-45473154 |
| 19:45498977-45499380 | 19:45529193-45529747 | 19:45616283-45616657 |
| 19:45640859-45641160 | 19:45952767-45953224 | 19:45952947-45953255 |
| 19:46411945-46414104 | 19:46648415-46648827 | 19:46829679-46829996 |
| 19:46970489-46970974 | 19:47267323-47267786 | 19:47309493-47311380 |
| 19:47429265-47429720 | 19:47447587-47448459 | 19:47456815-47457756 |
| 19:47572708-47573821 | 19:47660515-47660816 | 19:47950200-47950603 |
| 19:48061662-48062206 | 19:48126049-48126378 | 19:48341081-48341394 |
| 19:48405876-48406284 | 19:48415700-48416305 | 19:48596944-48597409 |
| 19:48629751-48630833 | 19:48978803-48979211 | 19:49042102-49042670 |
| 19:49050286-49050917 | 19:49071997-49072945 | 19:49132799-49133509 |
| 19:49422307-49423097 | 19:49432283-49432917 | 19:49433608-49434111 |
| 19:49436224-49436537 | 19:49442197-49442498 | 19:49527521-49528272 |
| 19:49689094-49690910 | 19:49843330-49844521 | 19:49999535-50000063 |
| 19:50040157-50040598 | 19:50050809-50051345 | 19:50202878-50203341 |
| 19:50605915-50606825 | 19:50607797-50608766 | 19:50666307-50668361 |
| 19:50728102-50728695 | 19:50756368-50756669 | 19:50817807-50818187 |
| 19:51132326-51132629 | 19:51184546-51185882 | 19:51200881-51201182 |
| 19:51719022-51719839 | 19:51809714-51812174 | 19:51887385-51888551 |
| 19:52369768-52370636 | 19:52453219-52454258 | 19:52492424-52493617 |
| 19:53037384-53038088 | 19:53057813-53058652 | 19:53131689-53133115 |
| 19:53197027-53197560 | 19:53254418-53255043 | 19:53279639-53283082 |
| 19:53308034-53308778 | 19:53693875-53695216 | 19:53712100-53712636 |
| 19:53719561-53721539 | 19:53898284-53898685 | 19:53899051-53899368 |
| 19:53908169-53908799 | 19:53928756-53929057 | 19:53941904-53942881 |
| 19:53962906-53963482 | 19:53977703-53978707 | 19:53979848-53980400 |
| 19:53981842-53982357 | 19:54064887-54065340 | 19:54286872-54289334 |
| 19:54312681-54314167 | 19:54372574-54375674 | 19:54502404-54502889 |
| 19:54628943-54629295 | 19:54881220-54881578 | 19:54881403-54881717 |
| 19:54882855-54884180 | 19:54958664-54960051 | 19:55160956-55162033 |
| 19:55433396-55434027 | 19:55440285-55440643 | 19:55493395-55494087 |
| 19:56217374-56218149 | 19:56367876-56368776 | 19:56393671-56394495 |
| 19:56476823-56478701 | 19:56507226-56508046 | 19:56538121-56539360 |
| 19:56637918-56638518 | 19:56642698-56644091 | 19:56660857-56661183 |
| 19:56671260-56672157 | 19:56765276-56765870 | 19:56795563-56795884 |
| 19:57069142-57069520 | 19:57106144-57107219 | 19:57526960-57527801 |
| 19:57583187-57584546 | 19:57708451-57709738 | 19:57829973-57830932 |
| 19:57886447-57888731 | 19:57934691-57935771 | 19:58044303-58044768 |
| 19:58058794-58060245 | 19:58117765-58118934 | 19:58395719-58397272 |
| 19:58440436-58441033 | 2:467701-470007 | 2:951193-952179 |
| 2:1166944-1168383 | 2:1261696-1262127 | 2:1591559-1592440 |
| 2:1721839-1722312 | 2:2291760-2292141 | 2:2567940-2569106 |
| 2:3223060-3223520 | 2:3469825-3470374 | 2:3703374-3704173 |
| 2:3782823-3783283 | 2:3806832-3808009 | 2:5690813-5691287 |
| 2:5692677-5694149 | 2:5695865-5696238 | 2:5697038-5697612 |
| 2:5981527-5981978 | 2:7435170-7436557 | 2:8219818-8220404 |
| 2:8551806-8552218 | 2:8595860-8596253 | 2:8675752-8676544 |
| 2:9092253-9092681 | 2:9287488-9288748 | 2:9565640-9565941 |
| 2:9665914-9666714 | 2:9699471-9699958 | 2:9702946-9703820 |
| 2:9757868-9758372 | 2:9914572-9915041 | 2:9992413-9992725 |
| 2:10091481-10091964 | 2:10260028-10260523 | 2:10266263-10266579 |
| 2:10936061-10936387 | 2:11562057-11562405 | 2:12106319-12106750 |
| 2:17877301-17878045 | 2:19356298-19357591 | 2:19361273-19362099 |
| 2:20448064-20448540 | 2:20490370-20490672 | 2:21636135-21636669 |
| 2:23663163-23663595 | 2:23670205-23670603 | 2:24085175-24085628 |
| 2:25103224-25103652 | 2:25216295-25216733 | 2:25276872-25277812 |
| 2:25347497-25347893 | 2:25789735-25790110 | 2:25955109-25955559 |
| 2:25981493-25982044 | 2:26299826-26300927 | 2:26493576-26493947 |
| 2:26685369-26686254 | 2:26805244-26805545 | 2:27078178-27078627 |
| 2:27258900-27260383 | 2:27380936-27381530 | 2:27622966-27623270 |
| 2:27735161-27735643 | 2:27761669-27761970 | 2:27798902-27799572 |
| 2:28273695-28276361 | 2:28296258-28296646 | 2:28341191-28341580 |
| 2:28810915-28811319 | 2:29022110-29022411 | 2:29330293-29330766 |
| 2:29748372-29750013 | 2:29919882-29920226 | 2:31579985-31581602 |
| 2:32947901-32948385 | 2:37323764-37324098 | 2:37378454-37378857 |
| 2:38074340-38075106 | 2:38095970-38096852 | 2:38960512-38960968 |
| 2:41698479-41698992 | 2:42459285-42459586 | 2:42492689-42493719 |
| 2:43158556-43159577 | 2:43832101-43832413 | 2:44768620-44769445 |
| 2:44800966-44803187 | 2:44927887-44929672 | 2:44932606-44934147 |
| 2:44942218-44943164 | 2:44944457-44945311 | 2:44953000-44953474 |
| 2:44958797-44960307 | 2:45003924-45005327 | 2:45170134-45170923 |
| 2:46167932-46168233 | 2:46286284-46286586 | 2:46804150-46804732 |
| 2:46836287-46836779 | 2:46909853-46910233 | 2:48175264-48175566 |
| 2:48423389-48423802 | 2:48755334-48755877 | 2:49974094-49974524 |
| 2:50346091-50348106 | 2:51026197-51027360 | 2:53961710-53962016 |
| 2:60853631-60854260 | 2:62288607-62288960 | 2:63055231-63055983 |
| 2:63861147-63861541 | 2:64454925-64455334 | 2:64594823-64595127 |
| 2:64838865-64840320 | 2:65320278-65320783 | 2:68645841-68646374 |
| 2:68732011-68732424 | 2:68838793-68839095 | 2:69770314-69770766 |
| 2:70887865-70889292 | 2:70904224-70905024 | 2:71023218-71024617 |
| 2:71465701-71466197 | 2:71548542-71548843 | 2:72146263-72147413 |
| 2:72919631-72920793 | 2:72924653-72926025 | 2:73202007-73203225 |
| 2:73291591-73292319 | 2:74414177-74415168 | 2:74497724-74498296 |
| 2:74499440-74500107 | 2:74515081-74516736 | 2:74647882-74648481 |
| 2:76409612-76410247 | 2:78721420-78721872 | 2:79511270-79511727 |
| 2:79512437-79512902 | 2:80302347-80303814 | 2:80322245-80322743 |
| 2:82023276-82023577 | 2:82267909-82268255 | 2:83874516-83875582 |
| 2:85067109-85067549 | 2:85779618-85780065 | 2:85886005-85887785 |
| 2:85894539-85894840 | 2:86695130-86695450 | 2:86789852-86790619 |
| 2:87449528-87449860 | 2:87583770-87584276 | 2:88452791-88453480 |
| 2:88764662-88765374 | 2:89611381-89612464 | 2:90360069-90361130 |
| 2:91441846-91442402 | 2:91442825-91443958 | 2:91685704-91686007 |
| 2:91685945-91686867 | 2:94735508-94736041 | 2:94998248-94998765 |
| 2:96115950-96116413 | 2:96175984-96176285 | 2:96343817-96344369 |
| 2:96556753-96557526 | 2:96806531-96807247 | 2:96858833-96859423 |
| 2:97845845-97846334 | 2:98087190-98087692 | 2:98346188-98347901 |
| 2:98766725-98767169 | 2:98909420-98909858 | 2:99300219-99300673 |
| 2:99663281-99663739 | 2:99701555-99702052 | 2:99821552-99821985 |
| 2:100284027-100284525 | 2:100292025-100292488 | 2:101053198-101053897 |
| 2:101232490-101232855 | 2:101630055-101630618 | 2:101696366-101697036 |
| 2:104658982-104659743 | 2:104863522-104864594 | 2:104867462-104868050 |
| 2:105396949-105397338 | 2:105754066-105757373 | 2:107986080-107986734 |
| 2:108377511-108378317 | 2:109125636-109126758 | 2:109231023-109231333 |
| 2:109275200-109276011 | 2:110102491-110102831 | 2:110675402-110676444 |
| 2:111366556-111367061 | 2:112181419-112182269 | 2:113499144-113500833 |
| 2:113542278-113542814 | 2:113978336-113978733 | 2:114662018-114662738 |
| 2:115160970-115161488 | 2:115162272-115162795 | 2:117859738-117860703 |
| 2:118185081-118185468 | 2:118223958-118224702 | 2:118309938-118310248 |
| 2:118841335-118843341 | 2:118844293-118846064 | 2:118849654-118850511 |
| 2:118857263-118859476 | 2:119157414-119159382 | 2:119252504-119252977 |
| 2:120442694-120443305 | 2:120576794-120577215 | 2:120654187-120654965 |
| 2:121284130-121284515 | 2:127371168-127371711 | 2:127696074-127696538 |
| 2:128319334-128319894 | 2:128437210-128437692 | 2:128536013-128536315 |
| 2:129318449-129318889 | 2:129992412-129993420 | 2:130005813-130006785 |
| 2:130213193-130213653 | 2:130428730-130429564 | 2:130691952-130692786 |
| 2:130916594-130917366 | 2:130963011-130963595 | 2:131300158-131300630 |
| 2:131330917-131331439 | 2:131353197-131353746 | 2:131424582-131425157 |
| 2:131444281-131444706 | 2:131460941-131461287 | 2:131646758-131647386 |
| 2:131812879-131813180 | 2:131829425-131830310 | 2:132037534-132038013 |
| 2:132037799-132038678 | 2:132257610-132258177 | 2:133266234-133267114 |
| 2:136764788-136765819 | 2:141330932-141331299 | 2:141514199-141514897 |
| 2:141951117-141951647 | 2:142129229-142130307 | 2:142872536-142873830 |
| 2:143383958-143384504 | 2:148875629-148876463 | 2:149039067-149039444 |
| 2:149141777-149142078 | 2:151361424-151362004 | 2:153871245-153872279 |
| 2:153872686-153873964 | 2:156321954-156322271 | 2:157257407-157258063 |
| 2:157466825-157467166 | 2:159616801-159617242 | 2:159797309-159797979 |
| 2:161416710-161419565 | 2:161422961-161424225 | 2:161427196-161428203 |
| 2:162838401-162838898 | 2:166239542-166239910 | 2:168244225-168244727 |
| 2:170711664-170712379 | 2:170818365-170819379 | 2:170966162-170966688 |
| 2:171763661-171763997 | 2:172080966-172081725 | 2:172087087-172089016 |
| 2:172108043-172108696 | 2:172250697-172251172 | 2:172380744-172381229 |
| 2:172558093-172558980 | 2:173191495-173191893 | 2:173359183-173359650 |
| 2:173749559-173750751 | 2:174326121-174327705 | 2:174659468-174659863 |
| 2:176068255-176068889 | 2:176075560-176075872 | 2:176081368-176085398 |
| 2:176099463-176100353 | 2:176108784-176109591 | 2:176112358-176113013 |
| 2:176115781-176117732 | 2:176119169-176119488 | 2:176123616-176125076 |
| 2:176128030-176128743 | 2:176132308-176133005 | 2:176137942-176138259 |
| 2:176147240-176148198 | 2:176149467-176150676 | 2:176151540-176152938 |
| 2:176164678-176165463 | 2:176178252-176178965 | 2:177337954-177338272 |
| 2:178323150-178323562 | 2:181682783-181683433 | 2:189178115-189180266 |
| 2:189613185-189613493 | 2:190843799-190845456 | 2:196593611-196593918 |
| 2:196927556-196928324 | 2:197786079-197787025 | 2:199461288-199462851 |
| 2:199464273-199467574 | 2:199468523-199470990 | 2:200462204-200462507 |
| 2:200473152-200473536 | 2:201975215-201975520 | 2:202171272-202171641 |
| 2:203935537-203937005 | 2:206274324-206275069 | 2:206641902-206642782 |
| 2:207681483-207681941 | 2:208123985-208124600 | 2:209579106-209579581 |
| 2:212536322-212536956 | 2:216017262-216017775 | 2:216535255-216535730 |
| 2:217511030-217511331 | 2:218231319-218231802 | 2:218356788-218357122 |
| 2:219331647-219332946 | 2:219418412-219419225 | 2:219569929-219570491 |
| 2:221999610-222000052 | 2:222289239-222289708 | 2:222296664-222297239 |
| 2:222303619-222305088 | 2:222311557-222312677 | 2:222320797-222321382 |
| 2:222733815-222734202 | 2:224084507-224086321 | 2:224952019-224952406 |
| 2:225464551-225464970 | 2:226797524-226798543 | 2:227388743-227389172 |
| 2:228180065-228180455 | 2:228181353-228182241 | 2:230973995-230974691 |
| 2:231308959-231309379 | 2:231605057-231605558 | 2:231614977-231615364 |
| 2:232351568-232352771 | 2:232385982-232388916 | 2:232419437-232421731 |
| 2:232485548-232486810 | 2:232487775-232488591 | 2:232503492-232504002 |
| 2:232568187-232568531 | 2:232928574-232929436 | 2:233186039-233186464 |
| 2:233421987-233422601 | 2:233982909-233983475 | 2:234335857-234337441 |
| 2:235644279-235644622 | 2:235670038-235670872 | 2:236048996-236049404 |
| 2:236163136-236164566 | 2:236168911-236169214 | 2:236168933-236170177 |
| 2:236172775-236173547 | 2:236236958-236237452 | 2:237626669-237627191 |
| 2:237648576-237648913 | 2:237649849-237650191 | 2:237786614-237786957 |
| 2:237888306-237888754 | 2:238059872-238060230 | 2:238235378-238235945 |
| 2:238561256-238561958 | 2:238847819-238848850 | 2:239129048-239129559 |
| 2:239298438-239298986 | 2:239599373-239599846 | 2:239762383-239762684 |
| 2:239822226-239822711 | 2:239936082-239937521 | 2:240042647-240042948 |
| 2:240290600-240291159 | 2:240455683-240456216 | 2:240645789-240646758 |
| 2:240672344-240673081 | 2:240797869-240798422 | 2:241540606-241542218 |
| 2:241724897-241725273 | 2:241774603-241775201 | 2:241803161-241803747 |
| 2:241819444-241820314 | 2:241866229-241866790 | 2:241901523-241902743 |
| 20:290968-291491 | 20:663266-664860 | 20:709832-710185 |
| 20:710258-710662 | 20:1150202-1150574 | 20:1313025-1313644 |
| 20:1871750-1872387 | 20:1892700-1893990 | 20:1946792-1947556 |
| 20:2558269-2559057 | 20:2620284-2620601 | 20:2693466-2693959 |
| 20:2820936-2821565 | 20:2839985-2840290 | 20:3174529-3175138 |
| 20:3239842-3240490 | 20:3667457-3668751 | 20:3697040-3697555 |
| 20:4247926-4248904 | 20:4507198-4507774 | 20:4687293-4687785 |
| 20:5065573-5066313 | 20:5316416-5317302 | 20:5514109-5514578 |
| 20:6838602-6839032 | 20:11889912-11890327 | 20:12012775-12013260 |
| 20:13331830-13332143 | 20:16821124-16821461 | 20:18058870-18059434 |
| 20:19880593-19880932 | 20:20369371-20370444 | 20:21020394-21020695 |
| 20:21100713-21102333 | 20:21105973-21106752 | 20:21391255-21391975 |
| 20:21395550-21396899 | 20:21397907-21398208 | 20:21507146-21508753 |
| 20:21521937-21523353 | 20:21705898-21706966 | 20:21713598-21714647 |
| 20:22568271-22568594 | 20:22568407-22568980 | 20:22576866-22580098 |
| 20:22581597-22583024 | 20:23035419-23036303 | 20:23193642-23194381 |
| 20:23692979-23694496 | 20:24953005-24953901 | 20:25036038-25036595 |
| 20:25080919-25084515 | 20:25147779-25148816 | 20:25453228-25453529 |
| 20:26207853-26209692 | 20:30299969-30300591 | 20:30316170-30317130 |
| 20:30894539-30894952 | 20:30899747-30900298 | 20:31560807-31561272 |
| 20:32030987-32031438 | 20:33852053-33852455 | 20:34268705-34269686 |
| 20:34476930-34477252 | 20:35541417-35541746 | 20:36173706-36174127 |
| 20:36435905-36436325 | 20:37293676-37295297 | 20:37295644-37297258 |
| 20:37598147-37598589 | 20:38386195-38386496 | 20:38601692-38602216 |
| 20:38674455-38674758 | 20:38674479-38674895 | 20:38723430-38724872 |
| 20:38726268-38726927 | 20:38727292-38728981 | 20:38764922-38765386 |
| 20:38806685-38807751 | 20:40688035-40688570 | 20:41365943-41366426 |
| 20:43916051-43916688 | 20:44247105-44247750 | 20:44670870-44671934 |
| 20:44810035-44810431 | 20:45097802-45098820 | 20:45316353-45317171 |
| 20:45890081-45890515 | 20:46012353-46013711 | 20:46028719-46030737 |
| 20:46057300-46058256 | 20:46357549-46357980 | 20:46458896-46459433 |
| 20:46811452-46811943 | 20:47457203-47457721 | 20:47701649-47702008 |
| 20:47725641-47725942 | 20:47795200-47795715 | 20:48384050-48384570 |
| 20:48767393-48768061 | 20:49270436-49270876 | 20:49318049-49319249 |
| 20:49420892-49421264 | 20:49566987-49567972 | 20:49595660-49597628 |
| 20:50009696-50010518 | 20:50047056-50048698 | 20:50234390-50234843 |
| 20:50339457-50340998 | 20:50959409-50959734 | 20:51162000-51163313 |
| 20:51366256-51367022 | 20:51400338-51400837 | 20:51802151-51802860 |
| 20:53774411-53775169 | 20:53844164-53844649 | 20:56004776-56005584 |
| 20:56587852-56588255 | 20:56804258-56804960 | 20:56925048-56925425 |
| 20:57076338-57076761 | 20:57173524-57173979 | 20:57264278-57265674 |
| 20:57457702-57458136 | 20:57999113-57999502 | 20:58514201-58514834 |
| 20:58514799-58515127 | 20:58649577-58650206 | 20:58797978-58798279 |
| 20:59316869-59317350 | 20:59452798-59453099 | 20:60087254-60087880 |
| 20:61390609-61390980 | 20:61965183-61965842 | 20:62193405-62194676 |
| 20:62252324-62252737 | 20:62501648-62502430 | 20:62709106-62709805 |
| 20:62836121-62836650 | 20:62974186-62974591 | 20:63005420-63006915 |
| 20:63014642-63015235 | 20:63039830-63040411 | 20:63084881-63085201 |
| 20:63176625-63178676 | 20:63318911-63319707 | 20:63360998-63362164 |
| 20:63487708-63489256 | 20:63624472-63624815 | 20:63682045-63682488 |
| 20:63828536-63830046 | 20:63832444-63833007 | 20:64256557-64256858 |
| 21:10520931-10522636 | 21:14216118-14216555 | 21:14541291-14542305 |
| 21:14643826-14644127 | 21:14995838-14996446 | 21:15164574-15164959 |
| 21:18244478-18244972 | 21:18752851-18753235 | 21:19129623-19130775 |
| 21:19997498-19997951 | 21:20270929-20271479 | 21:20996974-20998386 |
| 21:25338336-25339203 | 21:25963000-25963330 | 21:26140218-26140556 |
| 21:26572548-26573673 | 21:29130133-29131187 | 21:29938838-29940416 |
| 21:30479982-30480625 | 21:30598460-30599026 | 21:31529831-31530755 |
| 21:31559715-31560237 | 21:32595933-32597674 | 21:33024770-33025627 |
| 21:33026744-33027580 | 21:33047334-33047643 | 21:33395770-33396155 |
| 21:34693594-34693895 | 21:34807188-34809055 | 21:35160024-35160325 |
| 21:35887117-35887572 | 21:36207557-36207859 | 21:36246504-36246832 |
| 21:36692648-36694035 | 21:36696132-36698069 | 21:36700396-36701962 |
| 21:36704314-36704918 | 21:36712745-36713257 | 21:36723111-36723412 |
| 21:36909829-36910340 | 21:36990363-36990666 | 21:37005607-37006211 |
| 21:37193831-37194132 | 21:37509658-37510119 | 21:37915464-37916046 |
| 21:38277215-38278608 | 21:38802638-38803158 | 21:39392802-39393526 |
| 21:39726315-39728090 | 21:40846946-40847636 | 21:41506649-41507141 |
| 21:41508638-41510308 | 21:41664054-41664532 | 21:41668723-41669318 |
| 21:41763667-41763987 | 21:41899336-41901547 | 21:41921123-41922221 |
| 21:42040434-42040907 | 21:42195336-42195684 | 21:42227812-42228328 |
| 21:42474576-42475135 | 21:42665962-42666355 | 21:43657527-43658160 |
| 21:44349925-44350305 | 21:44368821-44370246 | 21:44417163-44418011 |
| 21:44557887-44559015 | 21:44733074-44735426 | 21:44965184-44965923 |
| 21:45073146-45073672 | 21:45286585-45287017 | 21:45370539-45371010 |
| 21:45477984-45479001 | 21:45589953-45590476 | 21:45615473-45616453 |
| 21:45864989-45865488 | 21:46297780-46298411 | 21:46347916-46348427 |
| 21:46477779-46478174 | 22:16581457-16583024 | 22:16600898-16602777 |
| 22:16608564-16610346 | 22:17119525-17120229 | 22:17219209-17221390 |
| 22:17370301-17370817 | 22:17370599-17370907 | 22:17890132-17891296 |
| 22:17950535-17951044 | 22:17996424-17997774 | 22:18999028-18999529 |
| 22:19148664-19149409 | 22:19150813-19151626 | 22:19523747-19524335 |
| 22:20013393-20013787 | 22:21021696-21021997 | 22:21023258-21023619 |
| 22:21031067-21031515 | 22:21360404-21360864 | 22:21416335-21416798 |
| 22:22404830-22405199 | 22:23179926-23180240 | 22:23292101-23292531 |
| 22:23453273-23453828 | 22:23768524-23768892 | 22:24424582-24425017 |
| 22:24495091-24495416 | 22:25025509-25025810 | 22:25281675-25283288 |
| 22:26356667-26357125 | 22:26756774-26757237 | 22:27299373-27299692 |
| 22:28680640-28681222 | 22:29060258-29060692 | 22:29307254-29307870 |
| 22:29388873-29389188 | 22:29479428-29482232 | 22:29744914-29745352 |
| 22:29938215-29938517 | 22:30561756-30563049 | 22:31660865-31661348 |
| 22:32073216-32073616 | 22:35446361-35446810 | 22:36510938-36511926 |
| 22:36541779-36542080 | 22:36839666-36840030 | 22:36990456-36991434 |
| 22:37069033-37069483 | 22:37324375-37325457 | 22:37334282-37335604 |
| 22:37362427-37362740 | 22:37496655-37497664 | 22:37613096-37613424 |
| 22:37805521-37805992 | 22:38080681-38081238 | 22:38088620-38089835 |
| 22:38306139-38306658 | 22:38463283-38463732 | 22:38973133-38973630 |
| 22:39024402-39024760 | 22:39044272-39044825 | 22:39591908-39592409 |
| 22:39662568-39663019 | 22:39664597-39665009 | 22:40678435-40678873 |
| 22:41195423-41195754 | 22:41238133-41238500 | 22:41636373-41636937 |
| 22:41699194-41699943 | 22:42498628-42499193 | 22:42500646-42501185 |
| 22:42695395-42695817 | 22:42752908-42754953 | 22:42766901-42767374 |
| 22:42970071-42970388 | 22:43035983-43037401 | 22:43301509-43301810 |
| 22:44081421-44081778 | 22:44360959-44361490 | 22:44955305-44955746 |
| 22:44979208-44980738 | 22:45866339-45867126 | 22:45968431-45969542 |
| 22:46262442-46263535 | 22:46547809-46549165 | 22:46616874-46618439 |
| 22:46621454-46621870 | 22:46626521-46628011 | 22:46726619-46727056 |
| 22:46842651-46843077 | 22:46903253-46903740 | 22:47129843-47130375 |
| 22:47205769-47206091 | 22:48300566-48301067 | 22:48309167-48309468 |
| 22:48488924-48489817 | 22:48576302-48576837 | 22:48594921-48595410 |
| 22:48630215-48630516 | 22:48780892-48781371 | 22:48899364-48899998 |
| 22:49091295-49091752 | 22:49449669-49450293 | 22:49632185-49632851 |
| 22:49938460-49938957 | 22:49983186-49984121 | 22:49999713-50000518 |
| 22:50014518-50015021 | 22:50057951-50058383 | 22:50089570-50090645 |
| 22:50170852-50171474 | 22:50237156-50237636 | 22:50266930-50267818 |
| 22:50480369-50481301 | 22:50604143-50604958 | 22:50759643-50760402 |
| 3:2900573-2901044 | 3:3798493-3798987 | 3:4995511-4996066 |
| 3:5070941-5071358 | 3:5095754-5096471 | 3:8767127-8767783 |
| 3:9151114-9151564 | 3:9552461-9553823 | 3:9642576-9642909 |
| 3:10196386-10197107 | 3:10205278-10206118 | 3:12503325-12503663 |
| 3:12749518-12750935 | 3:13203965-13204551 | 3:13281794-13283563 |
| 3:13801788-13802302 | 3:14809955-14810373 | 3:16066924-16067469 |
| 3:17478736-17479141 | 3:18438601-18440778 | 3:19148476-19148999 |
| 3:22372199-22373051 | 3:23672132-23672458 | 3:23738955-23739671 |
| 3:27723984-27724849 | 3:27731008-27731612 | 3:28434901-28435393 |
| 3:29440504-29440999 | 3:32107177-32107613 | 3:38028730-38029233 |
| 3:38039114-38039797 | 3:38605425-38607227 | 3:38633118-38634638 |
| 3:39808417-39808822 | 3:40615649-40616245 | 3:42265284-42266381 |
| 3:43037885-43038406 | 3:43105058-43105566 | 3:44554838-44555171 |
| 3:44712224-44712804 | 3:46808735-46809249 | 3:47579365-47580458 |
| 3:47884629-47884979 | 3:48140465-48140766 | 3:48465364-48465665 |
| 3:49132914-49133658 | 3:49166349-49166721 | 3:49685533-49686353 |
| 3:49718085-49719331 | 3:49906553-49906873 | 3:49930087-49931251 |
| 3:50429694-50429995 | 3:51955572-51956027 | 3:52452836-52455122 |
| 3:54047562-54047882 | 3:55607929-55608298 | 3:57017512-57018055 |
| 3:57981592-57984054 | 3:58272096-58272397 | 3:58586668-58587399 |
| 3:62170421-62170977 | 3:62376890-62378617 | 3:62874561-62875900 |
| 3:63278178-63278617 | 3:66037047-66037409 | 3:69766357-69766915 |
| 3:71493516-71494684 | 3:71496204-71496694 | 3:71548162-71549446 |
| 3:72625829-72626265 | 3:74614866-74615309 | 3:75965052-75965621 |
| 3:79766340-79766958 | 3:84958985-84959836 | 3:86012809-86013322 |
| 3:86949035-86949419 | 3:86990237-86991386 | 3:96350065-96350954 |
| 3:102053401-102053789 | 3:105353994-105354900 | 3:108053058-108053359 |
| 3:111978295-111978759 | 3:111998538-111999607 | 3:112185431-112185996 |
| 3:112304046-112304722 | 3:112497963-112500173 | 3:112629553-112630050 |
| 3:113625710-113626188 | 3:114505028-114505329 | 3:116444686-116445290 |
| 3:118850994-118852341 | 3:120780909-120781430 | 3:120908396-120909674 |
| 3:122184609-122185023 | 3:122921629-122922743 | 3:122975396-122977028 |
| 3:122979146-122979555 | 3:123419129-123420507 | 3:124582255-124582556 |
| 3:125769244-125771349 | 3:126213529-126213984 | 3:126394664-126395051 |
| 3:127608360-127608661 | 3:127710505-127710952 | 3:127817566-127818068 |
| 3:127914857-127915974 | 3:128075989-128077051 | 3:129001980-129004799 |
| 3:129045921-129046998 | 3:129592602-129593098 | 3:129656803-129657144 |
| 3:129974706-129975894 | 3:130147956-130148257 | 3:130183261-130183663 |
| 3:130517126-130517662 | 3:133674505-133676236 | 3:133746226-133746896 |
| 3:134020083-134021144 | 3:134406793-134407269 | 3:137761205-137762162 |
| 3:137764511-137765583 | 3:138435378-138436046 | 3:138694645-138695050 |
| 3:139309930-139310270 | 3:141411434-141413152 | 3:141797366-141798039 |
| 3:143998061-143998527 | 3:147378175-147378528 | 3:147388434-147389050 |
| 3:147390540-147391411 | 3:147391634-147392229 | 3:147392595-147393845 |
| 3:147395714-147397475 | 3:147398072-147398377 | 3:147405138-147410915 |
| 3:147412456-147412940 | 3:147421389-147421927 | 3:147422891-147424348 |
| 3:149221496-149221887 | 3:151265330-151266298 | 3:151460687-151461433 |
| 3:152835666-152836194 | 3:153371900-153372219 | 3:153881291-153881602 |
| 3:154120682-154121292 | 3:154121898-154122680 | 3:154122408-154123068 |
| 3:154427949-154429298 | 3:155665521-155665849 | 3:156290832-156291681 |
| 3:157542689-157543137 | 3:158094044-158096193 | 3:158101827-158104573 |
| 3:158570679-158570984 | 3:160404230-160404866 | 3:161851922-161852435 |
| 3:169938762-169939101 | 3:170258409-170258805 | 3:170330329-170331701 |
| 3:170419253-170420227 | 3:170585077-170586289 | 3:171002455-171002954 |
| 3:171167903-171168322 | 3:171824801-171825241 | 3:172071934-172072489 |
| 3:172447452-172448992 | 3:177378058-177378479 | 3:179897070-179897584 |
| 3:180036648-180037721 | 3:181695016-181695328 | 3:181695758-181696230 |
| 3:181696000-181696878 | 3:181712459-181713836 | 3:181726586-181728265 |
| 3:183161723-183162147 | 3:183370832-183371275 | 3:183905826-183906410 |
| 3:184381008-184381795 | 3:185473508-185473852 | 3:185656407-185657123 |
| 3:185689157-185689652 | 3:185711480-185711788 | 3:185826114-185826545 |
| 3:185826278-185826593 | 3:186193407-186194752 | 3:186360630-186361159 |
| 3:187219716-187220017 | 3:187227878-187228224 | 3:188165509-188166066 |
| 3:188212550-188213159 | 3:188316579-188317346 | 3:189986034-189986335 |
| 3:191345398-191345928 | 3:191639983-191642656 | 3:192514175-192515390 |
| 3:192893108-192893856 | 3:193245111-193245580 | 3:194204335-194205159 |
| 3:194322669-194323882 | 3:194397454-194398610 | 3:194540586-194541828 |
| 3:194598475-194599407 | 3:194687106-194687602 | 3:194802194-194802495 |
| 3:195543842-195544296 | 3:195618933-195620273 | 3:195664175-195664526 |
| 3:195689081-195690031 | 3:195983793-195984227 | 3:196121949-196123522 |
| 3:196337581-196337882 | 3:196338285-196339949 | 3:196758257-196758609 |
| 3:197367683-197367984 | 3:197394399-197395070 | 3:197482073-197482374 |
| 4:107116-108325 | 4:212145-213719 | 4:304546-305919 |
| 4:329717-330329 | 4:385926-387027 | 4:677673-678361 |
| 4:688813-689242 | 4:795123-796256 | 4:879474-880150 |
| 4:967356-968517 | 4:1278022-1278324 | 4:1323626-1324041 |
| 4:1330680-1332787 | 4:1339027-1339413 | 4:1402605-1409334 |
| 4:1413577-1415338 | 4:1548666-1548968 | 4:1792616-1792958 |
| 4:2803663-2804140 | 4:2817598-2818285 | 4:2937016-2937711 |
| 4:3402946-3403481 | 4:3746157-3747024 | 4:3766597-3767552 |
| 4:4226224-4226914 | 4:4660016-4660469 | 4:4689593-4689895 |
| 4:4857880-4858714 | 4:4871647-4872584 | 4:4969276-4969777 |
| 4:6222009-6222647 | 4:6245490-6246203 | 4:6447016-6447444 |
| 4:6568719-6569105 | 4:6753030-6753451 | 4:8041988-8042353 |
| 4:8290202-8290740 | 4:8614020-8614321 | 4:8617867-8618687 |
| 4:8891884-8892604 | 4:9324144-9324658 | 4:9498316-9498971 |
| 4:9781324-9782257 | 4:10456357-10456794 | 4:10461817-10462274 |
| 4:12042422-12042828 | 4:13523157-13525390 | 4:13529254-13529770 |
| 4:19457029-19457331 | 4:23888434-23888992 | 4:24799625-24800191 |
| 4:26093382-26094025 | 4:26731514-26732110 | 4:28402489-28403232 |
| 4:30718009-30718792 | 4:30721404-30723007 | 4:33648664-33649082 |
| 4:37889709-37890431 | 4:38758240-38759167 | 4:39319644-39319963 |
| 4:39446148-39447124 | 4:40284021-40284322 | 4:40750558-40751189 |
| 4:40890354-40891198 | 4:41745372-41748008 | 4:41873260-41873740 |
| 4:44447221-44448421 | 4:46992894-46994035 | 4:47706465-47707087 |
| 4:47838023-47838664 | 4:48134036-48134586 | 4:48483403-48484347 |
| 4:48490462-48492134 | 4:48985625-48985983 | 4:52076708-52077251 |
| 4:52679009-52679310 | 4:52751663-52751976 | 4:52861286-52861765 |
| 4:52861498-52861843 | 4:52862498-52863040 | 4:53905734-53906133 |
| 4:55793420-55793826 | 4:55819603-55820000 | 4:61200090-61201086 |
| 4:61201944-61202668 | 4:61202715-61203204 | 4:65668133-65669636 |
| 4:68433511-68434096 | 4:70844039-70844576 | 4:72631136-72631469 |
| 4:76251946-76252402 | 4:77617225-77617526 | 4:79408064-79408733 |
| 4:79650955-79651329 | 4:80263584-80264046 | 4:82733549-82734007 |
| 4:85127771-85128139 | 4:88277355-88277837 | 4:88736538-88738039 |
| 4:89303116-89304424 | 4:93834487-93834918 | 4:103719121-103719751 |
| 4:103719885-103720542 | 4:109989105-109989406 | 4:110612147-110612652 |
| 4:110618261-110619182 | 4:110628609-110629151 | 4:110630721-110631083 |
| 4:110633661-110634324 | 4:110639066-110639923 | 4:112511192-112511765 |
| 4:112521128-112521579 | 4:113049534-113049973 | 4:116926100-116927013 |
| 4:122826030-122826706 | 4:124206719-124207147 | 4:133150359-133151095 |
| 4:139438266-139438734 | 4:139610743-139611080 | 4:139735555-139736068 |
| 4:140092173-140092813 | 4:140497957-140498593 | 4:141131759-141132264 |
| 4:142565793-142566206 | 4:143616724-143617109 | 4:144644830-144645739 |
| 4:145623634-145623992 | 4:145806859-145808765 | 4:145812839-145813287 |
| 4:146222255-146222686 | 4:146632843-146633279 | 4:146639648-146640950 |
| 4:146655067-146655829 | 4:147825793-147826647 | 4:153137081-153137557 |
| 4:154489176-154490574 | 4:154907885-154908189 | 4:155208000-155208973 |
| 4:157221308-157223145 | 4:159278328-159278943 | 4:161377394-161377874 |
| 4:163343596-163343900 | 4:163343711-163344483 | 4:164383061-164384333 |
| 4:166313039-166314148 | 4:167233466-167233775 | 4:168437614-168437915 |
| 4:171812925-171814642 | 4:173500011-173500526 | 4:173506537-173507104 |
| 4:173515961-173516963 | 4:173518830-173519218 | 4:173526941-173527399 |
| 4:173528188-173528869 | 4:173530859-173531904 | 4:177595975-177596413 |
| 4:177699306-177699658 | 4:178632554-178633366 | 4:180057893-180058636 |
| 4:182322817-182324504 | 4:182547390-182548629 | 4:182631462-182631967 |
| 4:182643088-182644281 | 4:182849150-182849451 | 4:183823499-183823948 |
| 4:184420926-184421609 | 4:184848280-184849754 | 4:185128910-185129418 |
| 4:186095647-186096052 | 4:186840813-186841376 | 4:187063140-187064121 |
| 4:188282955-188283549 | 4:190015649-190017715 | 5:153270-153722 |
| 5:172887-173188 | 5:254928-255417 | 5:256204-257654 |
| 5:264287-264735 | 5:279358-279722 | 5:400157-400772 |
| 5:926323-926956 | 5:1021372-1021924 | 5:1204559-1204917 |
| 5:1243704-1244005 | 5:1262954-1263375 | 5:1295461-1296024 |
| 5:1335534-1336056 | 5:1386693-1386996 | 5:1394178-1394713 |
| 5:1512135-1512589 | 5:1518624-1519177 | 5:1536199-1536798 |
| 5:1571891-1572279 | 5:1597398-1597833 | 5:1857976-1858730 |
| 5:1875814-1877039 | 5:1877586-1877889 | 5:1878366-1879964 |
| 5:1881616-1882672 | 5:1886099-1888144 | 5:2002184-2003558 |
| 5:2738867-2739177 | 5:2748256-2751478 | 5:2752364-2753287 |
| 5:2754895-2755749 | 5:2756448-2757060 | 5:3203536-3204021 |
| 5:3590243-3591790 | 5:3594168-3594762 | 5:3595697-3597865 |
| 5:3599186-3602252 | 5:3605736-3607171 | 5:3659825-3660297 |
| 5:5078147-5079105 | 5:5139072-5139522 | 5:5183216-5183549 |
| 5:8906395-8906930 | 5:9376742-9377099 | 5:10562616-10563034 |
| 5:11384448-11384933 | 5:11903485-11903793 | 5:15499819-15500774 |
| 5:16179810-16180461 | 5:16575076-16575482 | 5:16701618-16701919 |
| 5:17216694-17217166 | 5:18466286-18466630 | 5:19315111-19315674 |
| 5:19988150-19988842 | 5:23303758-23304221 | 5:25190022-25190550 |
| 5:26327008-26327868 | 5:30494193-30494676 | 5:31449170-31449485 |
| 5:31663634-31665163 | 5:31909845-31910295 | 5:31995670-31996029 |
| 5:33936795-33938354 | 5:36680072-36680618 | 5:36722720-36723403 |
| 5:37836355-37837165 | 5:39446438-39447380 | 5:40680915-40681753 |
| 5:42756900-42757201 | 5:42943913-42944630 | 5:42949529-42950025 |
| 5:43061398-43061847 | 5:43711470-43711815 | 5:44388777-44389869 |
| 5:45695166-45696215 | 5:50427817-50429010 | 5:50440625-50441441 |
| 5:50969190-50969508 | 5:51383579-51384323 | 5:51399076-51399731 |
| 5:54518481-54519714 | 5:54985132-54985702 | 5:56480369-56480744 |
| 5:58457642-58457996 | 5:58751226-58751527 | 5:59039103-59040128 |
| 5:59276173-59277327 | 5:59716044-59716515 | 5:59746312-59746813 |
| 5:60429980-60430433 | 5:61319332-61319639 | 5:63887427-63887745 |
| 5:63958983-63959610 | 5:63960705-63961438 | 5:64165126-64165695 |
| 5:64505806-64506604 | 5:67003615-67004957 | 5:68071537-68071999 |
| 5:68266776-68267611 | 5:68543341-68544806 | 5:69238759-69239149 |
| 5:69491720-69492288 | 5:71718564-71719537 | 5:71719608-71719920 |
| 5:71960150-71960534 | 5:73303224-73303913 | 5:73382376-73382906 |
| 5:73419895-73420446 | 5:75054219-75054647 | 5:75566655-75567028 |
| 5:76468766-76470190 | 5:76954346-76954882 | 5:77144196-77144624 |
| 5:77367131-77368054 | 5:77628067-77628827 | 5:77636699-77637031 |
| 5:77638772-77639653 | 5:77640469-77641967 | 5:77645871-77646535 |
| 5:77843986-77845422 | 5:77906818-77907205 | 5:78908406-78908720 |
| 5:80034892-80035459 | 5:80668110-80668411 | 5:80960033-80960500 |
| 5:80961249-80961715 | 5:81232768-81233708 | 5:81852836-81853500 |
| 5:82365669-82366346 | 5:82931117-82931457 | 5:83778908-83779344 |
| 5:88671972-88673212 | 5:88678026-88678486 | 5:88685084-88685645 |
| 5:89730372-89730763 | 5:93603919-93604328 | 5:95730437-95730909 |
| 5:95841884-95844072 | 5:96431928-96433526 | 5:108216852-108217262 |
| 5:111070513-111070992 | 5:111735882-111736258 | 5:111932610-111933008 |
| 5:114055405-114056454 | 5:114361941-114363709 | 5:115179426-115179729 |
| 5:115815503-115817305 | 5:115961807-115962482 | 5:115963616-115963917 |
| 5:123094213-123095381 | 5:123098082-123099037 | 5:124629119-124629668 |
| 5:124735009-124735565 | 5:127290506-127290833 | 5:127410477-127410779 |
| 5:127602438-127603370 | 5:128271930-128272337 | 5:128536085-128538348 |
| 5:128964829-128965970 | 5:129459518-129460679 | 5:129461419-129461752 |
| 5:130994126-130995706 | 5:132251264-132251705 | 5:132257211-132259652 |
| 5:132451460-132451778 | 5:132655625-132656173 | 5:132777830-132778518 |
| 5:132819357-132819921 | 5:133944500-133945033 | 5:134113153-134113912 |
| 5:134460497-134460798 | 5:135027509-135029716 | 5:135031576-135032840 |
| 5:135038547-135039334 | 5:135050246-135050830 | 5:135308735-135309179 |
| 5:135399862-135400506 | 5:135489002-135489735 | 5:135491447-135492197 |
| 5:136365119-136365647 | 5:137888409-137889994 | 5:138303496-138303797 |
| 5:138467196-138467901 | 5:139014868-139015235 | 5:139543304-139543784 |
| 5:139669842-139670363 | 5:140108996-140109486 | 5:140631577-140632228 |
| 5:140794820-140797494 | 5:140800988-140803465 | 5:140807059-140809362 |
| 5:140821479-140823926 | 5:140834245-140836698 | 5:140841161-140842092 |
| 5:140848447-140848984 | 5:140855843-140856304 | 5:140868946-140871392 |
| 5:140875151-140877492 | 5:140881918-140884719 | 5:140925852-140928206 |
| 5:140966606-140967496 | 5:141040408-141041084 | 5:141051187-141051807 |
| 5:141094665-141094968 | 5:141121545-141121949 | 5:141135205-141137868 |
| 5:141149734-141152586 | 5:141224601-141225826 | 5:141234276-141234579 |
| 5:141245152-141245913 | 5:141338622-141341412 | 5:141343734-141344483 |
| 5:141354931-141355639 | 5:141369948-141370806 | 5:141373733-141374535 |
| 5:141375446-141376781 | 5:141381907-141385340 | 5:141387195-141390024 |
| 5:141397691-141398137 | 5:141397891-141400432 | 5:141407714-141408596 |
| 5:141413295-141415728 | 5:141417944-141420215 | 5:141420807-141421619 |
| 5:141430390-141433247 | 5:141484754-141485361 | 5:141811976-141812277 |
| 5:141876588-141877234 | 5:142215981-142216425 | 5:142362419-142362779 |
| 5:142834706-142835035 | 5:142960312-142960622 | 5:142977138-142978191 |
| 5:143008278-143009666 | 5:143244236-143244568 | 5:143261633-143262236 |
| 5:143311447-143311833 | 5:143405544-143406163 | 5:146334090-146334470 |
| 5:146339047-146340490 | 5:146342345-146343455 | 5:146345600-146346548 |
| 5:147310096-147310447 | 5:147508844-147509425 | 5:147509574-147510520 |
| 5:147661056-147661679 | 5:147968875-147969196 | 5:149724960-149725376 |
| 5:150086284-150087303 | 5:150155598-150156234 | 5:150166464-150167137 |
| 5:150189867-150190520 | 5:150302005-150302592 | 5:150317056-150317843 |
| 5:150599822-150600301 | 5:150904620-150905766 | 5:150946193-150947029 |
| 5:151034762-151035067 | 5:151158098-151158415 | 5:151202890-151203426 |
| 5:151303214-151304320 | 5:151924207-151925515 | 5:154473727-154474259 |
| 5:154593007-154593477 | 5:154850318-154851029 | 5:155729150-155729637 |
| 5:157651266-157652188 | 5:157671308-157672105 | 5:158075515-158075835 |
| 5:159051433-159051944 | 5:160231028-160232240 | 5:160271454-160271847 |
| 5:160370637-160371062 | 5:161847405-161848034 | 5:162067345-162067920 |
| 5:164600341-164601305 | 5:166978521-166978833 | 5:167309961-167310262 |
| 5:168529691-168530339 | 5:168791137-168792626 | 5:169350955-169351282 |
| 5:170140933-170141623 | 5:170861849-170862713 | 5:171152356-171152657 |
| 5:171307910-171310562 | 5:171312162-171312774 | 5:171313871-171317288 |
| 5:172174535-172174976 | 5:172407872-172408363 | 5:172414378-172414879 |
| 5:172420842-172421233 | 5:172462409-172462776 | 5:172657459-172658115 |
| 5:172670193-172670531 | 5:172772725-172773647 | 5:172800372-172801659 |
| 5:172896606-172898034 | 5:172900026-172900358 | 5:173233050-173234328 |
| 5:173244397-173245832 | 5:174121777-174122117 | 5:174327494-174327820 |
| 5:174731502-174732935 | 5:175687046-175687478 | 5:175772625-175773266 |
| 5:175871979-175872297 | 5:175872646-175873451 | 5:176203157-176203458 |
| 5:176366129-176366738 | 5:176618935-176620603 | 5:176679812-176680454 |
| 5:177116628-177117377 | 5:177131861-177133151 | 5:177320829-177321193 |
| 5:177950223-177951921 | 5:178286662-178287116 | 5:178499181-178499556 |
| 5:178535470-178535952 | 5:178576426-178577411 | 5:178940387-178940872 |
| 5:178940616-178941855 | 5:178990406-178990707 | 5:178994076-178994893 |
| 5:179059927-179061250 | 5:179428189-179428663 | 5:179618093-179619547 |
| 5:179801556-179802395 | 5:179816376-179816892 | 5:180089975-180090385 |
| 5:180658674-180659033 | 5:180673660-180674441 | 5:180816849-180818113 |
| 5:180873957-180874316 | 5:180911285-180911586 | 5:181059201-181059944 |
| 5:181100009-181100716 | 5:181114395-181115013 | 5:181164262-181164995 |
| 5:181173949-181174541 | 6:392190-393298 | 6:1379384-1379686 |
| 6:1384288-1385289 | 6:1392699-1393927 | 6:1606805-1608676 |
| 6:1615052-1615781 | 6:1618652-1620939 | 6:1788053-1788362 |
| 6:1895083-1895442 | 6:1904400-1904824 | 6:1952702-1953101 |
| 6:2314543-2314844 | 6:2498178-2498677 | 6:2852971-2853383 |
| 6:3032994-3033440 | 6:3388782-3390379 | 6:3592222-3592959 |
| 6:3749437-3750045 | 6:4078703-4079073 | 6:4853910-4854419 |
| 6:5025781-5026243 | 6:5121401-5121841 | 6:5456867-5457286 |
| 6:5764370-5764836 | 6:5851228-5851588 | 6:5994755-5996292 |
| 6:6002903-6005145 | 6:6224681-6225232 | 6:6397708-6398044 |
| 6:6478986-6479287 | 6:7051115-7051617 | 6:7987005-7987306 |
| 6:8435880-8436304 | 6:10384584-10385133 | 6:10408629-10410498 |
| 6:10882731-10884775 | 6:11009299-11009853 | 6:11044534-11045214 |
| 6:11143590-11144141 | 6:11317999-11318688 | 6:11368259-11368697 |
| 6:11772482-11773144 | 6:11824890-11825333 | 6:12749571-12750257 |
| 6:13269094-13269396 | 6:13508532-13509019 | 6:14094364-14095851 |
| 6:14542659-14543178 | 6:14732510-14733556 | 6:15062435-15063759 |
| 6:15266290-15266712 | 6:15400623-15401114 | 6:16333748-16335008 |
| 6:16355781-16356204 | 6:16583086-16583528 | 6:16587923-16588224 |
| 6:16964664-16965028 | 6:17015857-17016436 | 6:17599760-17600172 |
| 6:17907738-17909189 | 6:18276607-18277437 | 6:18490999-18491527 |
| 6:19691879-19692340 | 6:25053389-25053807 | 6:25910313-25910733 |
| 6:25930517-25931582 | 6:26016165-26018309 | 6:26119007-26119404 |
| 6:26240091-26240634 | 6:26250693-26252099 | 6:26270816-26271836 |
| 6:26613597-26614223 | 6:27260173-27260639 | 6:27267886-27268270 |
| 6:27290408-27291609 | 6:27545357-27545825 | 6:27631147-27631582 |
| 6:27679895-27681323 | 6:27809696-27810694 | 6:27814981-27815641 |
| 6:27823603-27824785 | 6:27873092-27873725 | 6:28207278-28208641 |
| 6:28259092-28259920 | 6:28553840-28554399 | 6:28765661-28766079 |
| 6:28785976-28786636 | 6:28810074-28810691 | 6:28813836-28814675 |
| 6:28825729-28826240 | 6:29553087-29554285 | 6:29850284-29850754 |
| 6:30127173-30127793 | 6:30171760-30172699 | 6:30260282-30260805 |
| 6:30778801-30779924 | 6:31182831-31183294 | 6:31728235-31729149 |
| 6:31889033-31889381 | 6:31900665-31901462 | 6:32095848-32096848 |
| 6:32151997-32153517 | 6:33007923-33009271 | 6:33139621-33140019 |
| 6:33631354-33631981 | 6:33771666-33772697 | 6:33975679-33976248 |
| 6:34466203-34466672 | 6:35213099-35213747 | 6:35297414-35298064 |
| 6:36285074-36285531 | 6:36355750-36356090 | 6:36381827-36382198 |
| 6:36840569-36841269 | 6:36849114-36849429 | 6:37024537-37025106 |
| 6:37033211-37033617 | 6:37535614-37536751 | 6:37783383-37784596 |
| 6:41333653-41335930 | 6:41354749-41355570 | 6:42727119-42728043 |
| 6:43283785-43285722 | 6:44653883-44654324 | 6:45663101-45663593 |
| 6:47736246-47736730 | 6:49738671-49738972 | 6:50714768-50716120 |
| 6:50720781-50721082 | 6:50724288-50724746 | 6:50819425-50820528 |
| 6:50823266-50823944 | 6:50850186-50850802 | 6:52307025-52307490 |
| 6:52423821-52424296 | 6:52757738-52758728 | 6:54308520-54308837 |
| 6:54846207-54846587 | 6:55174417-55175513 | 6:55578760-55579699 |
| 6:56064571-56064935 | 6:56875816-56876160 | 6:57799787-57800109 |
| 6:57813354-57813696 | 6:57822549-57823191 | 6:57853727-57854268 |
| 6:62285802-62286545 | 6:70426811-70427162 | 6:71419563-71419976 |
| 6:71886705-71887082 | 6:72182115-72183430 | 6:73236157-73236458 |
| 6:73314531-73315180 | 6:74468099-74468791 | 6:74474086-74474544 |
| 6:77463063-77463763 | 6:77464176-77464573 | 6:81761560-81763033 |
| 6:83430162-83430798 | 6:83707577-83708273 | 6:83852809-83853499 |
| 6:83853862-83854391 | 6:84762836-84764790 | 6:84772679-84773826 |
| 6:84967927-84968232 | 6:85126569-85127018 | 6:86937594-86937999 |
| 6:87968652-87969147 | 6:88167469-88167959 | 6:96015978-96016395 |
| 6:98824621-98825671 | 6:98827260-98827648 | 6:98831029-98831836 |
| 6:98842271-98843900 | 6:99590682-99590994 | 6:99602745-99603653 |
| 6:99608661- 99609451 | 6:99612820-99613313 | 6:99618380-99619444 |
| 6:100446996-100449895 | 6:100453346-100454489 | 6:100457375-100459070 |
| 6:100466922-100468157 | 6:100469063-100469736 | 6:104940129-104941277 |
| 6:104952854-104953514 | 6:105136124-105136643 | 6:105137141-105138109 |
| 6:105981041-105982117 | 6:105985909-105986668 | 6:105993416-105994371 |
| 6:106125831-106126549 | 6:106329625-106330108 | 6:106447923-106448237 |
| 6:107069858-107070160 | 6:107637890-107638900 | 6:108116646-108117365 |
| 6:108164957-108165720 | 6:108166793-108167871 | 6:108170037-108171676 |
| 6:108174032-108175678 | 6:108175407-108176237 | 6:108176436-108177011 |
| 6:108201021-108201337 | 6:108561579-108562602 | 6:108737007-108738310 |
| 6:109979184-109980199 | 6:110951978-110952305 | 6:111289811-111291424 |
| 6:111623218-111623519 | 6:116764428-116765135 | 6:117263212-117264886 |
| 6:117270295-117271070 | 6:117908380-117908798 | 6:122996346-122996881 |
| 6:123821980-123823374 | 6:125598337-125598711 | 6:128509050-128509362 |
| 6:133889020-133890047 | 6:134235821-134236122 | 6:134348194-134348821 |
| 6:137044559-137045360 | 6:137492207-137494472 | 6:138795563-138796656 |
| 6:139308233-139308535 | 6:141326443-141326854 | 6:141483576-141485010 |
| 6:142088092-142088953 | 6:142538264-142538566 | 6:142832882-142833352 |
| 6:142912898-142913983 | 6:143515420-143515879 | 6:144797765-144798203 |
| 6:146027488-146030138 | 6:146290133-146290837 | 6:146434199-146434868 |
| 6:147675213-147675714 | 6:148682255-148683637 | 6:149493583-149494081 |
| 6:149964689-149965554 | 6:151036697-151037718 | 6:154039060-154040023 |
| 6:154165496-154166608 | 6:154239393-154239884 | 6:156028299-156028753 |
| 6:156250656-156251004 | 6:157822332-157822828 | 6:157880982-157881502 |
| 6:157962758-157964428 | 6:158019055-158019373 | 6:158482010-158482371 |
| 6:158516084-158516581 | 6:158847067-158847368 | 6:158888335-158888644 |
| 6:158995335-158995649 | 6:159002533-159002862 | 6:160665599-160666625 |
| 6:160678819-160679787 | 6:163705909-163706376 | 6:163827973-163828458 |
| 6:163837613-163838104 | 6:166008686-166009416 | 6:166166983-166168888 |
| 6:166253148-166254203 | 6:166306653-166307428 | 6:166940615-166941073 |
| 6:166958169-166959746 | 6:167131212-167131768 | 6:167286318-167286768 |
| 6:167375802-167376103 | 6:167421136-167421950 | 6:167678158-167678683 |
| 6:168281468-168282079 | 6:168909064-168909815 | 6:170023140-170023753 |
| 6:170262333-170262999 | 6:170612481-170612837 | 7:289144-290421 |
| 7:290401-290886 | 7:353700-354073 | 7:652280-652702 |
| 7:711194-712298 | 7:768913-769431 | 7:907006-908147 |
| 7:915849-916278 | 7:1054852-1055485 | 7:1059184-1060446 |
| 7:1085857-1086210 | 7:1102967-1104026 | 7:1124923-1125362 |
| 7:1230405-1231886 | 7:1232083-1233099 | 7:1233942-1235069 |
| 7:1235546-1236112 | 7:1237700-1239184 | 7:1242107-1242923 |
| 7:1289677-1290265 | 7:1296947-1297938 | 7:1323142-1323952 |
| 7:2181437-2182893 | 7:2572087-2572618 | 7:2719967-2721323 |
| 7:2802717-2803395 | 7:2820437-2822399 | 7:4479273-4479723 |
| 7:4694966-4695389 | 7:4861511-4862243 | 7:5071918-5072572 |
| 7:5418371-5419040 | 7:5483796-5484311 | 7:5495683-5496268 |
| 7:5786367-5786894 | 7:6534399-6534765 | 7:6663791-6664694 |
| 7:6754055-6754572 | 7:6965242-6965544 | 7:6989767-6990237 |
| 7:8218327-8218628 | 7:8434442-8435766 | 7:8441038-8444178 |
| 7:9727069-9727825 | 7:10476284-10476835 | 7:10779239-10780233 |
| 7:11832234-11832751 | 7:12111619-12112025 | 7:12571275-12571941 |
| 7:12660448-12660864 | 7:16775709-16776010 | 7:17405700-17406041 |
| 7:19106181-19108021 | 7:19116279-19118593 | 7:19143400-19145560 |
| 7:20441912-20442219 | 7:20784073-20784861 | 7:22563066-22563503 |
| 7:23205788-23206793 | 7:24283833-24285611 | 7:24741916-24742270 |
| 7:25855982-25856437 | 7:25967337-25969169 | 7:26856572-26858357 |
| 7:27088077-27088899 | 7:27106203-27108941 | 7:27146726-27147712 |
| 7:27152618-27153208 | 7:27185436-27186397 | 7:27197381-27198660 |
| 7:27212684-27214271 | 7:27220289-27220989 | 7:27225391-27226102 |
| 7:27239256-27239886 | 7:27241146-27242219 | 7:27242348-27242676 |
| 7:27251697-27253146 | 7:27647016-27647982 | 7:27863879-27864250 |
| 7:27867959-27868503 | 7:28958579-28959244 | 7:29189898-29190408 |
| 7:29642794-29643095 | 7:29884118-29885292 | 7:29932074-29932407 |
| 7:30136359-30137397 | 7:31026771-31027292 | 7:31336083-31337121 |
| 7:32298142-32298661 | 7:32427939-32428730 | 7:32536879-32537231 |
| 7:32979234-32979679 | 7:33979725-33980380 | 7:35004020-35004378 |
| 7:35253133-35255767 | 7:35257282-35258549 | 7:35261400-35262344 |
| 7:35454540-35455272 | 7:37944136-37944508 | 7:38975757-38976204 |
| 7:39609518-39610356 | 7:39854863-39855308 | 7:42227771-42228374 |
| 7:44145486-44145795 | 7:44146214-44147980 | 7:44309641-44310353 |
| 7:45088371-45088783 | 7:46455831-46456336 | 7:47227943-47228304 |
| 7:47494582-47495290 | 7:48844755-48845108 | 7:49773680-49774050 |
| 7:49774895-49776302 | 7:50303306-50303846 | 7:50595810-50596112 |
| 7:52088433-52088753 | 7:54544401-54545063 | 7:54833201-54833560 |
| 7:55063654-55065504 | 7:55079315-55079734 | 7:55537471-55538900 |
| 7:56115741-56116784 | 7:56174413-56176182 | 7:56287132-56288198 |
| 7:57867928-57868328 | 7:63765378-63767337 | 7:64182993-64183570 |
| 7:64870197-64870636 | 7:64947079-64947559 | 7:67857869-67858170 |
| 7:67943531-67943832 | 7:71131637-71133576 | 7:72335513-72338198 |
| 7:72340884-72341401 | 7:72744545-72744892 | 7:73423951-73424261 |
| 7:73424095-73424635 | 7:73707217-73707721 | 7:73848943-73849401 |
| 7:74231205-74231630 | 7:74449924-74450378 | 7:74631531-74632031 |
| 7:75876509-75876814 | 7:75980038-75980492 | 7:76467724-76468141 |
| 7:77199676-77200106 | 7:78521692-78522338 | 7:80808922-80809315 |
| 7:81725791-81726227 | 7:82444334-82444753 | 7:86644555-86644983 |
| 7:87345945-87346495 | 7:90320736-90321187 | 7:90932815-90933161 |
| 7:92135126-92135584 | 7:93992324-93992669 | 7:94394250-94394711 |
| 7:95324309-95324898 | 7:96786266-96786719 | 7:96993185-96993601 |
| 7:97002219-97002638 | 7:97007086-97007389 | 7:97011685-97013242 |
| 7:97020770-97022463 | 7:97731817-97732281 | 7:97732287-97733524 |
| 7:98239612-98240077 | 7:98616345-98616747 | 7:99415297-99415770 |
| 7:99563668-99564154 | 7:99997385-99998152 | 7:100037237-100037538 |
| 7:100475462-100475924 | 7:100600713-100601014 | 7:100720547-100721104 |
| 7:100825355-100825871 | 7:100964657-100965676 | 7:101249268-101249716 |
| 7:101299475-101299898 | 7:101772843-101774096 | 7:102125070-102125794 |
| 7:102273934-102274359 | 7:102283672-102284886 | 7:102592525-102592854 |
| 7:102912697-102913208 | 7:103155635-103156009 | 7:103445114-103445835 |
| 7:105776074-105776466 | 7:106774629-106775155 | 7:107660404-107661099 |
| 7:112001063-112001592 | 7:116316743-116317848 | 7:116503255-116503717 |
| 7:116674632-116675137 | 7:117132548-117132949 | 7:117269348-117269826 |
| 7:117322215-117323576 | 7:117479097-117480060 | 7:122299681-122300800 |
| 7:122303763-122304382 | 7:123608043-123609296 | 7:123770451-123770835 |
| 7:128032483-128033263 | 7:128201244-128201866 | 7:128351693-128352785 |
| 7:128454699-128455000 | 7:128961329-128961729 | 7:128974334-128974849 |
| 7:130008737-130009169 | 7:130025864-130027312 | 7:130281246-130281547 |
| 7:130732865-130733873 | 7:130903371-130904063 | 7:131557894-131558665 |
| 7:132130309-132130861 | 7:133512459-133513011 | 7:134126795-134127101 |
| 7:134547813-134548172 | 7:134548790-134549666 | 7:134875896-134876342 |
| 7:135716807-135717229 | 7:136869767-136871730 | 7:137343342-137343697 |
| 7:137847216-137847926 | 7:138701155-138702067 | 7:138895659-138896179 |
| 7:139523934-139524392 | 7:139524101-139524422 | 7:141550837-141551145 |
| 7:141586011-141586400 | 7:142878869-142879398 | 7:143013856-143014248 |
| 7:143434705-143435220 | 7:149414999-149415550 | 7:149692391-149693130 |
| 7:149790360-149790720 | 7:150219418-150219759 | 7:150220528-150221297 |
| 7:150955957-150956278 | 7:151012640-151013781 | 7:151019055-151019511 |
| 7:151050741-151051133 | 7:151056900-151057397 | 7:151651568-151652132 |
| 7:151700760-151701190 | 7:151796193-151797002 | 7:152825884-152826299 |
| 7:152903486-152903987 | 7:152925295-152926017 | 7:153409236-153410326 |
| 7:153747907-153749896 | 7:153886084-153888647 | 7:154051761-154053576 |
| 7:154304954-154305477 | 7:154898145-154899991 | 7:155070581-155071617 |
| 7:155374465-155375229 | 7:155448480-155449640 | 7:155453656-155454315 |
| 7:155709861-155710177 | 7:155802766-155805780 | 7:156534997-156535309 |
| 7:156616873-156617361 | 7:157002526-157006953 | 7:157008576-157009143 |
| 7:157017847-157018760 | 7:157019212-157020541 | 7:157020858-157021525 |
| 7:157078185-157078674 | 7:157296949-157297611 | 7:157547029-157550179 |
| 7:157564853-157565511 | 7:157594166-157594841 | 7:157613040-157614105 |
| 7:157684134-157685072 | 7:157688956-157690032 | 7:157800206-157800698 |
| 7:158317729-158318300 | 7:158432854-158433513 | 7:158569512-158570487 |
| 7:158586721-158587024 | 7:158811385-158811800 | 7:158914935-158915579 |
| 7:158916753-158917356 | 7:158992463-158993230 | 7:159006839-159007140 |
| 7:159065601-159066684 | 7:159143602-159144603 | 7:159145585-159145895 |
| 8:736004-737277 | 8:866770-867337 | 8:978363-978836 |
| 8:1012685-1013812 | 8:1053880-1054346 | 8:1163006-1163746 |
| 8:1180545-1181133 | 8:1360054-1360435 | 8:1492956-1493595 |
| 8:1974799-1975307 | 8:1994216-1994627 | 8:2179059-2180186 |
| 8:2232609-2233802 | 8:4991205-4992321 | 8:4993926-4995117 |
| 8:6391647-6391995 | 8:6407289-6407699 | 8:7070835-7072224 |
| 8:7157572-7158944 | 8:8389845-8390443 | 8:8963122-8963752 |
| 8:9302903-9304367 | 8:9898519-9899223 | 8:9903171-9903754 |
| 8:9905003-9905533 | 8:10370751-10371248 | 8:10395416-10395784 |
| 8:10731192-10732366 | 8:11071326-11071791 | 8:11680397-11681987 |
| 8:11708050-11709116 | 8:11928358-11928660 | 8:15237421-15238320 |
| 8:17412857-17413978 | 8:18386797-18387569 | 8:18846587-18847294 |
| 8:19216775-19217209 | 8:19602301-19602883 | 8:20542677-20543022 |
| 8:22169958-22170365 | 8:22179365-22179767 | 8:22191068-22191531 |
| 8:22231839-22232419 | 8:22689763-22691316 | 8:22750889-22751365 |
| 8:23102735-23103279 | 8:23420740-23421172 | 8:23490119-23490439 |
| 8:23544323-23544746 | 8:23702085-23703031 | 8:23705023-23707812 |
| 8:23709542-23710723 | 8:24385205-24388090 | 8:24913775-24914716 |
| 8:25479239-25479755 | 8:26010179-26010764 | 8:26051605-26052173 |
| 8:26474133-26474564 | 8:26864056-26865365 | 8:27371760-27372349 |
| 8:27652415-27652849 | 8:28087381-28087846 | 8:28353187-28353694 |
| 8:29320136-29320467 | 8:29347952-29348399 | 8:29352818-29353434 |
| 8:29828347-29828842 | 8:30056356-30057703 | 8:30575795-30576096 |
| 8:30890425-30890740 | 8:33599999-33600336 | 8:35682572-35683798 |
| 8:37717138-37717963 | 8:37798609-37798952 | 8:37965783-37967162 |
| 8:38404132-38404433 | 8:38510006-38510485 | 8:38553969-38554412 |
| 8:39107440-39108145 | 8:41308163-41309166 | 8:41830606-41831173 |
| 8:42325760-42326212 | 8:42498927-42499415 | 8:43090934-43091400 |
| 8:48303093-48303406 | 8:48570715-48571016 | 8:49071759-49072496 |
| 8:49909459-49910311 | 8:49910901-49912180 | 8:52565111-52565588 |
| 8:52565787-52566429 | 8:52939258-52940916 | 8:53250569-53252179 |
| 8:53692810-53693239 | 8:53879074-53879570 | 8:53906862-53907248 |
| 8:54457331-54459979 | 8:54466701-54467699 | 8:56112798-56114326 |
| 8:56156980-56157523 | 8:56320013-56320490 | 8:56445418-56448049 |
| 8:56477209-56478225 | 8:57041678-57041979 | 8:57209506-57210937 |
| 8:57213982-57214689 | 8:60922445-60923375 | 8:61202317-61202701 |
| 8:62701623-62702199 | 8:64368808-64370675 | 8:64372451-64373907 |
| 8:64377009-64379395 | 8:64585775-64587435 | 8:65929357-65929852 |
| 8:66133550-66133852 | 8:66456227-66456676 | 8:66960833-66961581 |
| 8:67824539-67825343 | 8:68330378-68331095 | 8:70034476-70035478 |
| 8:70069551-70070278 | 8:70072242-70072896 | 8:71556056-71557262 |
| 8:71831336-71832688 | 8:71845432-71846794 | 8:76681785-76682307 |
| 8:78762201-78762621 | 8:79610936-79611555 | 8:80300953-80301475 |
| 8:80348160-80349844 | 8:84184376-84185044 | 8:85438082-85439117 |
| 8:86661662-86662151 | 8:89760032-89760457 | 8:90082094-90082619 |
| 8:94638772-94639208 | 8:94641786-94642447 | 8:94664856-94665199 |
| 8:94891048-94891532 | 8:95073059-95073653 | 8:97277260-97277704 |
| 8:97844243-97844952 | 8:97918160-97918635 | 8:98427550-98428403 |
| 8:98942853-98943154 | 8:98948881-98949817 | 8:98972141-98974807 |
| 8:100105505-100106499 | 8:100303047-100304176 | 8:100443020-100443753 |
| 8:100458638-100460054 | 8:101097761-101098089 | 8:101222945-101224890 |
| 8:101492133-101495401 | 8:102236658-102237119 | 8:102576860-102577309 |
| 8:102976961-102977293 | 8:105319203-105320182 | 8:106657296-106658787 |
| 8:108083613-108083916 | 8:109229117-109229593 | 8:113046440-113046989 |
| 8:113407585-113408918 | 8:113431929-113432423 | 8:115467950-115468258 |
| 8:117866470-117866835 | 8:118904021-118905650 | 8:118952332-118952881 |
| 8:119564928-119565337 | 8:120970969-120971353 | 8:121322816-121323293 |
| 8:121574264-121574657 | 8:121600441-121600782 | 8:122733857-122734361 |
| 8:122830041-122830443 | 8:123148967-123149344 | 8:123154677-123154994 |
| 8:123548225-123548579 | 8:124629425-124630054 | 8:124728667-124729348 |
| 8:124943770-124946195 | 8:125032221-125032648 | 8:125832967-125833403 |
| 8:127740507-127741004 | 8:127795621-127796117 | 8:127852855-127853368 |
| 8:127859594-127860362 | 8:127870994-127871442 | 8:127876090-127876909 |
| 8:127931530-127932342 | 8:127980432-127981682 | 8:129272247-129273777 |
| 8:129981665-129982614 | 8:130439516-130440006 | 8:133559419-133559900 |
| 8:136369696-136370208 | 8:137851377-137852387 | 8:139462195-139462497 |
| 8:139909926-139910575 | 8:140090166-140090524 | 8:140149656-140149987 |
| 8:140310095-140310463 | 8:140621270-140621619 | 8:140745416-140745717 |
| 8:140773978-140774343 | 8:141282613-141282951 | 8:141305601-141305916 |
| 8:141379147-141379582 | 8:141518194-141519031 | 8:142320822-142321238 |
| 8:142416899-142417247 | 8:142710053-142711013 | 8:142776510-142777316 |
| 8:143085220-143085690 | 8:143159158-143159823 | 8:143183770-143184071 |
| 8:143245905-143246532 | 8:143554128-143554562 | 8:143601975-143602435 |
| 8:143798283-143798584 | 8:143841351-143842283 | 8:144266775-144267246 |
| 8:144331665-144331969 | 8:144333089-144333809 | 8:144335540-144336422 |
| 8:144353679-144354632 | 8:144474707-144475692 | 8:144580700-144581501 |
| 8:144699699-144700645 | 8:144766689-144767046 | 9:72427-73132 |
| 9:116665-118681 | 9:463242-464269 | 9:621240-622235 |
| 9:698301-698690 | 9:738255-738585 | 9:842333-843576 |
| 9:970601-970904 | 9:970673-972159 | 9:1045298-1046565 |
| 9:2159685-2160154 | 9:2241038-2241772 | 9:2241486-2241809 |
| 9:2802334-2802758 | 9:3180766-3181188 | 9:4345037-4345355 |
| 9:5621269-5621636 | 9:6412111-6412675 | 9:6902387-6902741 |
| 9:7477432-7478458 | 9:14992009-14992559 | 9:16062769-16063290 |
| 9:16144403-16144837 | 9:17906475-17907604 | 9:19786804-19787269 |
| 9:19788375-19789553 | 9:21966286-21968866 | 9:21970766-21971308 |
| 9:21989626-21990256 | 9:23820576-23821220 | 9:23822192-23822861 |
| 9:23826154-23826679 | 9:23850764-23851122 | 9:32782810-32783777 |
| 9:33164523-33165736 | 9:34457229-34457615 | 9:34623721-34624912 |
| 9:34666048-34666560 | 9:35689396-35690065 | 9:36739513-36739912 |
| 9:36873911-36875746 | 9:36946706-36947059 | 9:37002513-37002943 |
| 9:37026077-37026511 | 9:37035422-37036585 | 9:37911789-37912177 |
| 9:37926126-37926573 | 9:39030541-39031065 | 9:39078654-39079222 |
| 9:41073586-41074364 | 9:41124631-41125175 | 9:41126798-41127101 |
| 9:41266606-41267059 | 9:41663421-41663936 | 9:62830796-62831373 |
| 9:62833433-62834185 | 9:63859334-63860257 | 9:65283399-65284332 |
| 9:65736745-65738366 | 9:68302767-68304630 | 9:68307052-68307928 |
| 9:70043288-70043813 | 9:75903912-75904273 | 9:77013846-77016340 |
| 9:77018807-77019971 | 9:77455255-77455743 | 9:77647031-77647851 |
| 9:80062806-80063115 | 9:84140415-84140861 | 9:84271507-84271987 |
| 9:84712046-84712457 | 9:87567640-87568146 | 9:87605167-87606268 |
| 9:88652057-88652823 | 9:89334531-89334885 | 9:90755441-90756352 |
| 9:91117295-91118234 | 9:92100479-92100878 | 9:92147519-92148187 |
| 9:93948399-93949472 | 9:94332425-94333004 | 9:94882994-94883322 |
| 9:94900281-94900585 | 9:95348959-95350109 | 9:95552937-95553256 |
| 9:95553062-95553445 | 9:96027302-96027855 | 9:96145987-96146331 |
| 9:96719680-96720411 | 9:96877104-96877851 | 9:97076739-97077801 |
| 9:97222402-97222866 | 9:97529661-97530160 | 9:97851485-97852194 |
| 9:97854892-97855346 | 9:98709382-98710054 | 9:98943287-98943808 |
| 9:99368951-99369290 | 9:99579719-99580408 | 9:101737318-101738207 |
| 9:104906777-104907168 | 9:105521006-105521308 | 9:107586402-107587648 |
| 9:109167200-109167738 | 9:110116482-110116784 | 9:110252140-110252768 |
| 9:110933731-110934685 | 9:112028526-112029287 | 9:112737842-112738143 |
| 9:112817890-112818295 | 9:113059581-113060373 | 9:113062187-113063158 |
| 9:113089101-113090507 | 9:113488518-113488819 | 9:115000009-115000691 |
| 9:117718369-117719958 | 9:117744930-117745620 | 9:120928121-120928459 |
| 9:121071645-121073437 | 9:121556192-121556565 | 9:121597879-121598502 |
| 9:121736139-121737495 | 9:122214336-122214899 | 9:123282636-123283009 |
| 9:123329379-123329832 | 9:124015383-124015686 | 9:124239240-124239600 |
| 9:124242830-124243152 | 9:124477933-124478460 | 9:124501864-124503460 |
| 9:124505095-124505936 | 9:125380420-125380920 | 9:125660019-125660344 |
| 9:126028224-126028525 | 9:126480176-126480765 | 9:127877873-127878315 |
| 9:127952074-127952414 | 9:128607524-128608009 | 9:128863789-128864279 |
| 9:128920707-128921184 | 9:128921218-128922559 | 9:129170370-129170836 |
| 9:129344698-129345492 | 9:129446797-129447173 | 9:129495529-129495976 |
| 9:129574919-129575220 | 9:129623344-129625286 | 9:130286364-130286703 |
| 9:130658999-130659837 | 9:130918915-130919302 | 9:130949047-130949348 |
| 9:131034727-131035028 | 9:131728643-131729974 | 9:132164098-132164518 |
| 9:132513243-132513841 | 9:132580649-132581266 | 9:132582637-132583490 |
| 9:132987923-132988285 | 9:133115517-133116181 | 9:133162924-133163429 |
| 9:133738497-133738809 | 9:134500985-134501409 | 9:134564404-134564969 |
| 9:134912321-134912622 | 9:135380015-135380386 | 9:135438544-135439551 |
| 9:135905033-135905553 | 9:135908267-135908695 | 9:135926726-135928365 |
| 9:136050282-136051282 | 9:136193107-136194152 | 9:136199224-136201132 |
| 9:136202113-136203102 | 9:136228036-136228552 | 9:136364052-136364361 |
| 9:136385911-136386448 | 9:136821363-136822160 | 9:136844972-136846524 |
| 9:136977560-136978184 | 9:136985857-136986278 | 9:137048499-137049987 |
| 9:137138688-137139913 | 9:137145598-137146384 | 9:137156462-137157039 |
| 9:137160872-137161597 | 9:137510468-137511688 | 9:137564996-137565421 |
| 9:137760977-137761354 | 9:137876863-137878402 | 9:137878849-137879182 |
| 9:138022741-138023522 | | |

Table 1A—ccRCC

The DMRs may be those disclosed in Table 1 of GB2215319.1 and which are also available at: https://doi.org/10.5281/zenodo.10001239 as of 17 Oct. 2023. The entire contents of Table 1 of GB2215319.1 and https://doi.org/10.5281/zenodo.10001239 are expressly incorporated herein by reference as “Table 1A”.

TABLE 2
GBM

| | | |
|---|---|---|
| 1:917994-918947 | 1:943466-944986 | 1:961663-962626 |
| 1:964498-967795 | 1:1006248-1008508 | 1:1010543-1012281 |
| 1:1046322-1048761 | 1:1081111-1083176 | 1:1083965-1085896 |
| 1:1087064-1088052 | 1:1125718-1128974 | 1:1129969-1133259 |
| 1:1160801-1170679 | 1:1177770-1181076 | 1:1181567-1184003 |
| 1:1196979-1199123 | 1:1254739-1255736 | 1:1262398-1263300 |
| 1:1312314-1313286 | 1:1313223-1314531 | 1:1320596-1321577 |
| 1:1377252-1378207 | 1:1541689-1544397 | 1:1551335-1552306 |
| 1:1563775-1565213 | 1:1622167-1623158 | 1:1624600-1625598 |
| 1:1670943-1671919 | 1:1753735-1754725 | 1:1974644-1976766 |
| 1:2027762-2031193 | 1:2042672-2043627 | 1:2058890-2062544 |
| 1:2079000-2081542 | 1:2084022-2084978 | 1:2091176-2092591 |
| 1:2096233-2097180 | 1:2103126-2108722 | 1:2109108-2110856 |
| 1:2126876-2129343 | 1:2129946-2131683 | 1:2160565-2161473 |
| 1:2164602-2165807 | 1:2167759-2174489 | 1:2176771-2177741 |
| 1:2179923-2180912 | 1:2183265-2184191 | 1:2184305-2185278 |
| 1:2187592-2189100 | 1:2201747-2202691 | 1:2239290-2240224 |
| 1:2247976-2249800 | 1:2258899-2260737 | 1:2273247-2274957 |
| 1:2304467-2305370 | 1:2307672-2309530 | 1:2309692-2310625 |
| 1:2316829-2319629 | 1:2319602-2322041 | 1:2327346-2328989 |
| 1:2350620-2352143 | 1:2352277-2354224 | 1:2386716-2387631 |
| 1:2403008-2406982 | 1:2429896-2431474 | 1:2440067-2441038 |
| 1:2495671-2498032 | 1:2502080-2504396 | 1:2504258-2506543 |
| 1:2516959-2517941 | 1:2550873-2553023 | 1:2568834-2569811 |
| 1:2632388-2634361 | 1:2808053-2808984 | 1:2819564-2820545 |
| 1:2847582-2848519 | 1:2902886-2907885 | 1:2928047-2929013 |
| 1:2929735-2932405 | 1:3083936-3084855 | 1:3093936-3094898 |
| 1:3111798-3112764 | 1:3121109-3122011 | 1:3162054-3163167 |
| 1:3165800-3167521 | 1:3193716-3194635 | 1:3194744-3195718 |
| 1:3211924-3214533 | 1:3236089-3237011 | 1:3240210-3242009 |
| 1:3242550-3243452 | 1:3244338-3246390 | 1:3391241-3392539 |
| 1:3403580-3405095 | 1:3405251-3406196 | 1:3406751-3407664 |
| 1:3411483-3412466 | 1:3425233-3426590 | 1:3432929-3436962 |
| 1:3460758-3462346 | 1:3464217-3469670 | 1:3495866-3496853 |
| 1:3508528-3511569 | 1:3518934-3519922 | 1:3540701-3542519 |
| 1:3542860-3545695 | 1:3549765-3550740 | 1:3557261-3558253 |
| 1:3586376-3587287 | 1:3605671-3608979 | 1:3627133-3628130 |
| 1:3631663-3632973 | 1:3634786-3635694 | 1:3639019-3640664 |
| 1:3641031-3642124 | 1:3642453-3643951 | 1:3644755-3645678 |
| 1:3673409-3674360 | 1:3814535-3815528 | 1:3821225-3822207 |
| 1:3855463-3856407 | 1:3905241-3908342 | 1:3913938-3915282 |
| 1:4707794-4708720 | 1:5858109-5860916 | 1:5862220-5864382 |
| 1:5866854-5867779 | 1:5882409-5883834 | 1:5895335-5896310 |
| 1:5896857-5899648 | 1:5910900-5912540 | 1:6069577-6073874 |
| 1:6077378-6078321 | 1:6127522-6129695 | 1:6131310-6132512 |
| 1:6224286-6225570 | 1:6305702-6307110 | 1:6360150-6362509 |
| 1:6469101-6470596 | 1:6504624-6507013 | 1:6592899-6593890 |
| 1:6621065-6622017 | 1:6627999-6628967 | 1:7059545-7061381 |
| 1:7491313-7492240 | 1:7639981-7640960 | 1:7769592-7770895 |
| 1:7984466-7985629 | 1:8325302-8327045 | 1:8351547-8352498 |
| 1:8356310-8357229 | 1:8360677-8361657 | 1:8455414-8456316 |
| 1:8944402-8946305 | 1:9005469-9006371 | 1:9014292-9015281 |
| 1:9247121-9248071 | 1:9264100-9265032 | 1:9348747-9350725 |
| 1:9606043-9606989 | 1:9699748-9700747 | 1:9730446-9731348 |
| 1:10540835-10541737 | 1:10550576-10551478 | 1:10615696-10617305 |
| 1:10621747-10622700 | 1:10623289-10624879 | 1:10639355-10640337 |
| 1:10645969-10648494 | 1:10659510-10661203 | 1:10666122-10667120 |
| 1:10670101-10674330 | 1:10963168-10965657 | 1:11054229-11055544 |
| 1:11188529-11190239 | 1:11674099-11675001 | 1:12191115-12192404 |
| 1:12192418-12193364 | 1:12228953-12230216 | 1:12506618-12507593 |
| 1:15048029-15049565 | 1:15092687-15093645 | 1:15436773-15439020 |
| 1:15742282-15745333 | 1:15933970-15935403 | 1:15946242-15947144 |
| 1:15991886-15992982 | 1:16115382-16116286 | 1:16134375-16135351 |
| 1:16137440-16138415 | 1:16724130-16725824 | 1:16868806-16869708 |
| 1:16924614-16925548 | 1:16975913-16978353 | 1:17203266-17205258 |
| 1:17232636-17233538 | 1:17338884-17339850 | 1:17507945-17508890 |
| 1:18882682-18883870 | 1:18890526-18892862 | 1:20138772-20140133 |
| 1:21434990-21435945 | 1:21847469-21848446 | 1:21883754-21884713 |
| 1:21884620-21885560 | 1:21895771-21896737 | 1:22119782-22120765 |
| 1:22129009-22130254 | 1:22284638-22286045 | 1:22864625-22865599 |
| 1:23192968-23194015 | 1:23433848-23434784 | 1:23435521-23438002 |
| 1:23800976-23802625 | 1:24450505-24451424 | 1:24464214-24465116 |
| 1:24916340-24917305 | 1:25585929-25586874 | 1:25649476-25650384 |
| 1:25891488-25893247 | 1:26553265-26554969 | 1:26729285-26730273 |
| 1:26950948-26951897 | 1:27005248-27007281 | 1:27359339-27361749 |
| 1:27391992-27392985 | 1:27522178-27523081 | 1:27547519-27548501 |
| 1:27550735-27552442 | 1:27686285-27687274 | 1:29118419-29120123 |
| 1:29349580-29350506 | 1:30720597-30722190 | 1:30723022-30724987 |
| 1:30776179-30777081 | 1:30936481-30937420 | 1:31756006-31756956 |
| 1:32273700-32276483 | 1:32360893-32363697 | 1:32768340-32770214 |
| 1:32888642-32889631 | 1:33258118-33259047 | 1:33276206-33277203 |
| 1:33624543-33625509 | 1:34756866-34759080 | 1:34789141-34790075 |
| 1:34976068-34977891 | 1:36087065-36088366 | 1:36099622-36102188 |
| 1:36465921-36467353 | 1:36481733-36484583 | 1:37731937-37732848 |
| 1:38026824-38027793 | 1:39369622-39370524 | 1:39632407-39633510 |
| 1:40816022-40817302 | 1:41017444-41018382 | 1:41020970-41023608 |
| 1:41582395-41583811 | 1:42151847-42154009 | 1:42916735-42917723 |
| 1:43005101-43006003 | 1:43272305-43274677 | 1:43284506-43287023 |
| 1:43308537-43309439 | 1:43527020-43527975 | 1:43589577-43590971 |
| 1:43597304-43598250 | 1:44016632-44018402 | 1:44146582-44147506 |
| 1:44640099-44641665 | 1:44653992-44655501 | 1:44688903-44689809 |
| 1:44767022-44767940 | 1:44805557-44812133 | 1:45645614-45646581 |
| 1:46404877-46406072 | 1:46444729-46445651 | 1:46447315-46449531 |
| 1:46612751-46613731 | 1:47022538-47023506 | 1:47434130-47435085 |
| 1:47991926-47995420 | 1:48760471-48761373 | 1:51594308-51595289 |
| 1:52030317-52031219 | 1:53091332-53093729 | 1:53275911-53276869 |
| 1:54257005-54257955 | 1:54260518-54261420 | 1:54558695-54559597 |
| 1:54622893-54625173 | 1:54850357-54852183 | 1:54865161-54866063 |
| 1:54979682-54981834 | 1:55055793-55056962 | 1:58814461-58816797 |
| 1:61088195-61089139 | 1:62263043-62264233 | 1:63703146-63704068 |
| 1:63731354-63732256 | 1:84397933-84399648 | 1:85581526-85583691 |
| 1:86467616-86469382 | 1:86551916-86554206 | 1:90726077-90727023 |
| 1:91545705-91547630 | 1:91948132-91950480 | 1:92217301-92218814 |
| 1:93588106-93589827 | 1:93692570-93693476 | 1:94094792-94095736 |
| 1:100700090-100700992 | 1:107570054-107571038 | 1:108649493-108650394 |
| 1:109258084-109259061 | 1:109762422-109765741 | 1:110222638-110224161 |
| 1:111225764-111228547 | 1:112523988-112524911 | 1:113978202-113980574 |
| 1:115767628-115770518 | 1:116400465-116401367 | 1:117932893-117933821 |
| 1:118999127-119000105 | 1:119006774-119007757 | 1:119622163-119623065 |
| 1:119896253-119898123 | 1:147022910-147023812 | 1:147049750-147051526 |
| 1:147611029-147612689 | 1:147757578-147760205 | 1:147772664-147775188 |
| 1:147901999-147903292 | 1:147907295-147909747 | 1:150720314-150721457 |
| 1:151046207-151047706 | 1:153000779-153001932 | 1:153057586-153058726 |
| 1:153310205-153311969 | 1:153565479-153567467 | 1:153792579-153793489 |
| 1:154570124-154572367 | 1:154707000-154708680 | 1:155032155-155035328 |
| 1:155057078-155058064 | 1:155125799-155126800 | 1:155196550-155198039 |
| 1:155282052-155283492 | 1:155320091-155322091 | 1:155334997-155335968 |
| 1:155883200-155884180 | 1:155961322-155962606 | 1:156060485-156061842 |
| 1:156077022-156078016 | 1:156135498-156136708 | 1:156617403-156618713 |
| 1:156873271-156874262 | 1:157100354-157101770 | 1:157127912-157128814 |
| 1:157924905-157927125 | 1:159854203-159856515 | 1:160794984-160796167 |
| 1:161983285-161984236 | 1:162178181-162179115 | 1:162364934-162368102 |
| 1:162377732-162379476 | 1:162380716-162382684 | 1:163422241-163424354 |
| 1:164575334-164578333 | 1:165827009-165828001 | 1:166988533-166990303 |
| 1:167205832-167206734 | 1:167255538-167256440 | 1:167912696-167913847 |
| 1:173917384-173918306 | 1:175044360-175045788 | 1:179818292-179819252 |
| 1:179825528-179826498 | 1:180568043-180568945 | 1:181482236-181483224 |
| 1:182055103-182060091 | 1:186378107-186379034 | 1:198679194-198680164 |
| 1:200211623-200212525 | 1:200212415-200214363 | 1:200464791-200465693 |
| 1:201212245-201213297 | 1:201223908-201224882 | 1:201225534-201227547 |
| 1:201280528-201281503 | 1:201361085-201362051 | 1:201518831-201520074 |
| 1:201718189-201719153 | 1:202009300-202011338 | 1:202015025-202015939 |
| 1:202317565-202319268 | 1:202438187-202439184 | 1:202646160-202647062 |
| 1:202884091-202884993 | 1:203061180-203062082 | 1:203085236-203086206 |
| 1:203177725-203181235 | 1:203229131-203231631 | 1:203347579-203348526 |
| 1:203485647-203488548 | 1:204561725-204562665 | 1:204996411-204997919 |
| 1:205056220-205058557 | 1:205339216-205340144 | 1:206511546-206512517 |
| 1:207452418-207453792 | 1:207675429-207676331 | 1:209172715-209173695 |
| 1:209232167-209233871 | 1:209790017-209790919 | 1:209826462-209827728 |
| 1:210242063-210243003 | 1:211204189-211205097 | 1:211325169-211329358 |
| 1:213997112-213998551 | 1:214415196-214416098 | 1:218323825-218325014 |
| 1:220879990-220883131 | 1:220894936-220896887 | 1:221457740-221458642 |
| 1:221738419-221739342 | 1:222036671-222037573 | 1:222071335-222072267 |
| 1:222543255-222544715 | 1:222736166-222737855 | 1:226186982-226188054 |
| 1:226641458-226642606 | 1:226982199-226983184 | 1:227542601-227543554 |
| 1:227557877-227558908 | 1:227617398-227618300 | 1:227883253-227884155 |
| 1:228110161-228111063 | 1:228273834-228277237 | 1:228313660-228319106 |
| 1:228424822-228426868 | 1:229107387-229108289 | 1:229302451-229303403 |
| 1:229593825-229594919 | 1:229658866-229659768 | 1:230263164-230264271 |
| 1:230278069-230280424 | 1:230746190-230748350 | 1:230801233-230802135 |
| 1:231335866-231336852 | 1:232983754-232984749 | 1:234481533-234482435 |
| 1:234742240-234743227 | 1:236013043-236013945 | 1:236347460-236348532 |
| 1:236482469-236483371 | 1:236559313-236560275 | 1:236736536-236737525 |
| 1:239907130-239909749 | 1:243488103-243489962 | 1:243929982-243931107 |
| 1:244035939-244036926 | 1:244054530-244055490 | 1:245334936-245336965 |
| 1:245576910-245578448 | 1:245591452-245592836 | 1:245702486-245703388 |
| 1:246090362-246091529 | 1:246788195-246792425 | 1:247036754-247037690 |
| 1:247423401-247424625 | 1:247515876-247517404 | 1:247531520-247532459 |
| 10:267387-268413 | 10:326678-327664 | 10:340268-342044 |
| 10:347872-348811 | 10:357191-358162 | 10:389589-390561 |
| 10:417989-420934 | 10:471737-473213 | 10:479329-482701 |
| 10:485373-487018 | 10:565811-566979 | 10:583919-584868 |
| 10:593457-596135 | 10:604636-607921 | 10:638959-640563 |
| 10:661992-662934 | 10:690304-691937 | 10:696801-699258 |
| 10:809398-810361 | 10:1016697-1017609 | 10:1022099-1023001 |
| 10:1026267-1027169 | 10:1036799-1037718 | 10:1083974-1084882 |
| 10:1101314-1102264 | 10:1162873-1164542 | 10:1172320-1174001 |
| 10:1204627-1207379 | 10:1216458-1217763 | 10:1341354-1342257 |
| 10:1405338-1406239 | 10:1463441-1464414 | 10:1466498-1468417 |
| 10:1517874-1518919 | 10:1551921-1554426 | 10:1625912-1627237 |
| 10:1662273-1663904 | 10:1682451-1684438 | 10:1701390-1702377 |
| 10:1746714-1747693 | 10:1786159-1787299 | 10:1952494-1953482 |
| 10:1992602-1993632 | 10:2013854-2014956 | 10:2098970-2099890 |
| 10:2314579-2315766 | 10:3116032-3116940 | 10:3142971-3144003 |
| 10:3147581-3148483 | 10:3150210-3151759 | 10:3234379-3235334 |
| 10:3270571-3271538 | 10:3322511-3323665 | 10:3330735-3332701 |
| 10:3376299-3377268 | 10:3470192-3471112 | 10:3516743-3517714 |
| 10:3931372-3932335 | 10:5498160-5500206 | 10:5512354-5513305 |
| 10:5639752-5640713 | 10:5893539-5894949 | 10:6093156-6094103 |
| 10:6236436-6237338 | 10:6255400-6256396 | 10:6321592-6322558 |
| 10:6488890-6489819 | 10:7096526-7097495 | 10:7172030-7172999 |
| 10:7367599-7368501 | 10:7942611-7943582 | 10:8014179-8015081 |
| 10:9406467-9407414 | 10:9407696-9408785 | 10:11343853-11345373 |
| 10:11443002-11444285 | 10:11462022-11464756 | 10:11473494-11474439 |
| 10:11744559-11745461 | 10:11783222-11784343 | 10:11891658-11892961 |
| 10:11893625-11894622 | 10:12174045-12174974 | 10:12824017-12824958 |
| 10:12997256-12998157 | 10:13208862-13209764 | 10:13708230-13709132 |
| 10:14773668-14776682 | 10:15211348-15213402 | 10:17304634-17305936 |
| 10:21387550-21388452 | 10:21506729-21509636 | 10:21520680-21521621 |
| 10:22333396-22337281 | 10:24950139-24951450 | 10:26642048-26643579 |
| 10:28795403-28796369 | 10:29490835-29491748 | 10:29522107-29523075 |
| 10:30068650-30069551 | 10:30691881-30692854 | 10:31317742-31319752 |
| 10:31321288-31322256 | 10:34941395-34942334 | 10:35212091-35213050 |
| 10:35420539-35421441 | 10:37680085-37681072 | 10:38450375-38451475 |
| 10:42833138-42834056 | 10:43103381-43105947 | 10:43172121-43173023 |
| 10:43342138-43343086 | 10:43666788-43669379 | 10:43938478-43939384 |
| 10:46018894-46020680 | 10:46045256-46047377 | 10:46577533-46578510 |
| 10:47368047-47369654 | 10:48502414-48503322 | 10:48671268-48672226 |
| 10:48745319-48746549 | 10:48913299-48915797 | 10:48938466-48939430 |
| 10:49297797-49299714 | 10:49325419-49327565 | 10:49407948-49408850 |
| 10:49607560-49611055 | 10:49610901-49611884 | 10:50026718-50027684 |
| 10:50248244-50249596 | 10:52954936-52955838 | 10:60887747-60888884 |
| 10:61000990-61002592 | 10:62103314-62104233 | 10:62812716-62814136 |
| 10:68722005-68722908 | 10:69022241-69023200 | 10:69416642-69417950 |
| 10:70551690-70552765 | 10:71318369-71319285 | 10:71711352-71712714 |
| 10:72007806-72008951 | 10:73640940-73643444 | 10:73805927-73806875 |
| 10:73914775-73915703 | 10:75028942-75029886 | 10:75098903-75099805 |
| 10:75238130-75239120 | 10:76107830-76108732 | 10:76149673-76150619 |
| 10:76875248-76876160 | 10:76886251-76888230 | 10:76915933-76917044 |
| 10:77976117-77977019 | 10:78236409-78237807 | 10:78336782-78337717 |
| 10:78633761-78634718 | 10:79381281-79383098 | 10:79383452-79384354 |
| 10:84184477-84185379 | 10:86138370-86139469 | 10:86534916-86536375 |
| 10:92044594-92046641 | 10:93756580-93758678 | 10:93992624-93994621 |
| 10:95550030-95550932 | 10:96303759-96305117 | 10:96359461-96360962 |
| 10:96606438-96607340 | 10:98382432-98384161 | 10:100748636- |
| | | 100749538 |
| 10:101032359- | 10:101651442- | 10:101739821- |
| 101033261 | 101652365 | 101741354 |
| 10:102054823- | 10:103040350- | 10:103277066- |
| 102055815 | 103041280 | 103278589 |
| 10:103457723- | 10:103601726- | 10:103823239- |
| 103459861 | 103603497 | 103824141 |
| 10:103838251- | 10:103858901- | 10:104314760- |
| 103839207 | 103859804 | 104316483 |
| 10:104333362- | 10:110227583- | 10:110400606- |
| 104336883 | 110228485 | 110401508 |
| 10:110684509- | 10:111104520- | 10:112820714- |
| 110685644 | 111105499 | 112821793 |
| 10:112831463- | 10:113164938- | 10:114402510- |
| 112832513 | 113166442 | 114403413 |
| 10:114657518- | 10:119042033- | 10:119050198- |
| 114659489 | 119043032 | 119051133 |
| 10:119341495- | 10:119748810- | 10:119817912- |
| 119342461 | 119749773 | 119819798 |
| 10:121733024- | 10:122229775- | 10:122464462- |
| 121734021 | 122230751 | 122465396 |
| 10:123890725- | 10:124622823- | 10:125023921- |
| 123893425 | 124624386 | 125028408 |
| 10:125150846- | 10:125762955- | 10:126387141- |
| 125151826 | 125763948 | 126389991 |
| 10:127178120- | 10:127280761- | 10:127934193- |
| 127179022 | 127281734 | 127935649 |
| 10:128708026- | 10:128721009- | 10:129021981- |
| 128710982 | 128722171 | 129022883 |
| 10:129045361- | 10:129403712- | 10:129541724- |
| 129046675 | 129404614 | 129543316 |
| 10:129613780- | 10:129625822- | 10:129646394- |
| 129614682 | 129627543 | 129647375 |
| 10:129655494- | 10:129680629- | 10:129768470- |
| 129656415 | 129681531 | 129771849 |
| 10:129777114- | 10:129793679- | 10:129839774- |
| 129779817 | 129795172 | 129844005 |
| 10:129866614- | 10:129887321- | 10:129932733- |
| 129868583 | 129890996 | 129933839 |
| 10:129968582- | 10:130026866- | 10:130046172- |
| 129969567 | 130027789 | 130047153 |
| 10:131074911- | 10:131739546- | 10:131929198- |
| 131076047 | 131740525 | 131930161 |
| 10:131936385- | 10:131970278- | 10:132066528- |
| 131937480 | 131971956 | 132067507 |
| 10:132103273- | 10:132114497- | 10:132124155- |
| 132105400 | 132119466 | 132125630 |
| 10:132135189- | 10:132205561- | 10:132208557- |
| 132137914 | 132208539 | 132209504 |
| 10:132260230- | 10:132299399- | 10:132347542- |
| 132261138 | 132300552 | 132349047 |
| 10:132350667- | 10:132402439- | 10:132412305- |
| 132351664 | 132404013 | 132413564 |
| 10:132414259- | 10:132415676- | 10:132440810- |
| 132415173 | 132416630 | 132442560 |
| 10:132465092- | 10:132484608- | 10:132488553- |
| 132466071 | 132485781 | 132491493 |
| 10:132492820- | 10:132544025- | 10:132546922- |
| 132493722 | 132546782 | 132551098 |
| 10:132605720- | 10:132652639- | 10:132658081- |
| 132606699 | 132654283 | 132659050 |
| 10:132677278- | 10:132682725- | 10:132725792- |
| 132678234 | 132683678 | 132729425 |
| 10:132733501- | 10:132734536- | 10:132739153- |
| 132734472 | 132735485 | 132740905 |
| 10:132742207- | 10:132745531- | 10:132756846- |
| 132743533 | 132747307 | 132757804 |
| 10:132759219- | 10:132767802- | 10:132776746- |
| 132760184 | 132768769 | 132777683 |
| 10:132778380- | 10:132779186- | 10:132781999- |
| 132779282 | 132780807 | 132783293 |
| 10:132798106- | 10:132837570- | 10:132847467- |
| 132799681 | 132839726 | 132852183 |
| 10:132856351- | 10:132866832- | 10:132909461- |
| 132859864 | 132871132 | 132912034 |
| 10:132915024- | 10:132918845- | 10:132924264- |
| 132917907 | 132924172 | 132930582 |
| 10:132963375- | 10:132976721- | 10:132992005- |
| 132965389 | 132978649 | 132994490 |
| 10:133012025- | 10:133017767- | 10:133076062- |
| 133017203 | 133026846 | 133078055 |
| 10:133079348- | 10:133094568- | 10:133126540- |
| 133082470 | 133095551 | 133127535 |
| 10:133164819- | 10:133167951- | 10:133169659- |
| 133167230 | 133169367 | 133171432 |
| 10:133214687- | 10:133268309- | 10:133269241- |
| 133217536 | 133269290 | 133270788 |
| 10:133272764- | 10:133288124- | 10:133292652- |
| 133273736 | 133289108 | 133294293 |
| 10:133311605- | 10:133344902- | 10:133377301- |
| 133312507 | 133347857 | 133378939 |
| 10:133388514- | 10:133462152- | 10:133567674- |
| 133389437 | 133463054 | 133568938 |
| 11:206430-207863 | 11:291102-292443 | 11:324859-325762 |
| 11:390494-395069 | 11:403479-404994 | 11:460040-461004 |
| 11:476168-477155 | 11:477310-478393 | 11:486551-487738 |
| 11:527738-528702 | 11:589308-590255 | 11:593214-594190 |
| 11:604930-605905 | 11:607470-608430 | 11:610574-614023 |
| 11:614310-616129 | 11:618365-620340 | 11:621636-622618 |
| 11:623265-629226 | 11:719071-721394 | 11:723980-724968 |
| 11:791672-792638 | 11:803925-804880 | 11:823717-824705 |
| 11:839316-841037 | 11:968470-969437 | 11:970114-972600 |
| 11:972375-973368 | 11:985431-986408 | 11:1005515-1007959 |
| 11:1034304-1035539 | 11:1050198-1051175 | 11:1089374-1090330 |
| 11:1160856-1162948 | 11:1193699-1195769 | 11:1197028-1199636 |
| 11:1240403-1242184 | 11:1252239-1253451 | 11:1253606-1255964 |
| 11:1271010-1272527 | 11:1276722-1281129 | 11:1283312-1284214 |
| 11:1303954-1307884 | 11:1455931-1457421 | 11:1471175-1472104 |
| 11:1498870-1500492 | 11:1514162-1515115 | 11:1647529-1649624 |
| 11:1803512-1804477 | 11:1827631-1828557 | 11:1835761-1841756 |
| 11:1879993-1880958 | 11:1935714-1939406 | 11:2087884-2088965 |
| 11:2171269-2173038 | 11:2188281-2191446 | 11:2211442-2214715 |
| 11:2264046-2267358 | 11:2350439-2351573 | 11:2384606-2385555 |
| 11:2448910-2450404 | 11:2476696-2478611 | 11:2520640-2522160 |
| 11:2561930-2563922 | 11:2572112-2575103 | 11:2719260-2720241 |
| 11:2758289-2759254 | 11:2760738-2763146 | 11:2898008-2902799 |
| 11:2907687-2908589 | 11:2920262-2921481 | 11:3105665-3106982 |
| 11:3119933-3120955 | 11:3142852-3143921 | 11:3164772-3168515 |
| 11:3224368-3225652 | 11:5440778-5441681 | 11:5929673-5930651 |
| 11:5938144-5939521 | 11:6496351-6498640 | 11:6610331-6613107 |
| 11:6640571-6641522 | 11:7519345-7520574 | 11:9710061-9710963 |
| 11:10567812-10569904 | 11:12159424-12160364 | 11:12285926-12288534 |
| 11:15653687-15654589 | 11:16826253-16828468 | 11:17387114-17390725 |
| 11:17544493-17545807 | 11:17772388-17773318 | 11:17883796-17884766 |
| 11:18708684-18709586 | 11:20126158-20127060 | 11:23402949-23404430 |
| 11:23405128-23406122 | 11:30579785-30581992 | 11:31505549-31506494 |
| 11:31825800-31828227 | 11:34075812-34076909 | 11:34193143-34194045 |
| 11:34512482-34514554 | 11:35260017-35260919 | 11:36272254-36273178 |
| 11:36381210-36382158 | 11:43521175-43523029 | 11:44563567-44565504 |
| 11:45234863-45236099 | 11:45668395-45669355 | 11:45694876-45697182 |
| 11:45720807-45722755 | 11:45805604-45806574 | 11:45810722-45811687 |
| 11:45905555-45908467 | 11:45927710-45929596 | 11:46290952-46291854 |
| 11:46385015-46387300 | 11:46680374-46681278 | 11:47217487-47218389 |
| 11:49101594-49103102 | 11:57304458-57305360 | 11:58904864-58906554 |
| 11:60416591-60417492 | 11:60933897-60935579 | 11:61008376-61009357 |
| 11:61342131-61343373 | 11:61479226-61481900 | 11:61534112-61535527 |
| 11:61609896-61610857 | 11:61742117-61745015 | 11:61754513-61755452 |
| 11:61778615-61779566 | 11:61780028-61780930 | 11:61812913-61813815 |
| 11:61898632-61899541 | 11:62355962-62356864 | 11:62504968-62506388 |
| 11:62706753-62707957 | 11:62909510-62910484 | 11:63490199-63491169 |
| 11:63811528-63812475 | 11:63911082-63912715 | 11:64102597-64103507 |
| 11:64117572-64119518 | 11:64258011-64258991 | 11:64367663-64368639 |
| 11:64369513-64372385 | 11:64408985-64409942 | 11:64505070-64506252 |
| 11:64555071-64556486 | 11:64561460-64562822 | 11:64754259-64755214 |
| 11:64826510-64827935 | 11:64829070-64830042 | 11:64832188-64833130 |
| 11:65204144-65205058 | 11:65400543-65401445 | 11:65528801-65529770 |
| 11:65867241-65869027 | 11:65917258-65918223 | 11:66021032-66021965 |
| 11:66215640-66217191 | 11:66285287-66286275 | 11:66333110-66338159 |
| 11:66341142-66342126 | 11:66420502-66421720 | 11:66686582-66687722 |
| 11:66858132-66859119 | 11:66860175-66864516 | 11:67070922-67072413 |
| 11:67249817-67250789 | 11:67432121-67433066 | 11:67438143-67439506 |
| 11:67453597-67456234 | 11:67458143-67459102 | 11:67501502-67502474 |
| 11:67502813-67507412 | 11:67522520-67524592 | 11:67674058-67676157 |
| 11:68047043-68048384 | 11:68049296-68050583 | 11:68311702-68314757 |
| 11:68347883-68348851 | 11:68370287-68373276 | 11:68423091-68424825 |
| 11:68439213-68440397 | 11:68446305-68447274 | 11:68773154-68774520 |
| 11:68806652-68808456 | 11:68914362-68915343 | 11:68933933-68935317 |
| 11:68939877-68940858 | 11:69205446-69206390 | 11:69403394-69404296 |
| 11:69439263-69440183 | 11:69475513-69476504 | 11:69592713-69593673 |
| 11:69809369-69810330 | 11:70020483-70021476 | 11:70128117-70129081 |
| 11:70414743-70415695 | 11:70491779-70492908 | 11:70527659-70528798 |
| 11:70718521-70719654 | 11:70737556-70738845 | 11:70788325-70789305 |
| 11:70797193-70798862 | 11:70819873-70821178 | 11:70824241-70825206 |
| 11:70826334-70827329 | 11:71130532-71131957 | 11:71423476-71424699 |
| 11:71438389-71439805 | 11:71471608-71472548 | 11:71472945-71473902 |
| 11:71490318-71492298 | 11:71582329-71584224 | 11:72228666-72229655 |
| 11:72280567-72281491 | 11:72821026-72821961 | 11:73296168-73298295 |
| 11:73977010-73977912 | 11:74591368-74592283 | 11:77033475-77034838 |
| 11:77100363-77103743 | 11:77184273-77185182 | 11:77470554-77471456 |
| 11:78022609-78024457 | 11:78207225-78208127 | 11:78209003-78210767 |
| 11:85934171-85935598 | 11:86672385-86674163 | 11:94566961-94567892 |
| 11:94791499-94792432 | 11:108222060- | 11:108538158- |
| | 108223047 | 108539812 |
| 11:114160624- | 11:114960532- | 11:115759271- |
| 114161539 | 114961520 | 115761417 |
| 11:116820601- | 11:117191825- | 11:117280769- |
| 116822174 | 117192823 | 117281671 |
| 11:117478120- | 11:117518211- | 11:117670274- |
| 117479043 | 117519491 | 117671177 |
| 11:118892514- | 11:119664379- | 11:120121524- |
| 118894849 | 119665355 | 120125497 |
| 11:120167031- | 11:120563631- | 11:120590457- |
| 120169109 | 120564697 | 120591399 |
| 11:121003603- | 11:121362499- | 11:121603009- |
| 121004506 | 121363485 | 121603911 |
| 11:122776844- | 11:122804187- | 11:123166634- |
| 122777746 | 122805089 | 123167576 |
| 11:123576908- | 11:124200398- | 11:125062259- |
| 123578444 | 124201373 | 125064468 |
| 11:125128966- | 11:125162978- | 11:125681297- |
| 125130017 | 125165755 | 125682799 |
| 11:125746345- | 11:126162532- | 11:126374666- |
| 125747444 | 126163434 | 126375618 |
| 11:128457220- | 11:128685969- | 11:128841848- |
| 128458122 | 128686908 | 128843488 |
| 11:128866794- | 11:129366988- | 11:130001500- |
| 128867856 | 129367910 | 130002466 |
| 11:130123116- | 11:130142664- | 11:130205702- |
| 130124087 | 130144699 | 130206936 |
| 11:130208207- | 11:130415993- | 11:130486263- |
| 130210374 | 130416966 | 130487246 |
| 11:132284338- | 11:133360511- | 11:134150335- |
| 132285328 | 133363665 | 134151282 |
| 11:134152893- | 11:134156121- | 11:134345655- |
| 134153883 | 134157511 | 134346815 |
| 11:134383166- | 12:137544-140411 | 12:186262-187170 |
| 134385941 | | |
| 12:224470-225605 | 12:262629-264104 | 12:556112-557107 |
| 12:932330-933306 | 12:1243204-1244106 | 12:1499276-1501124 |
| 12:1628418-1629811 | 12:1645107-1647438 | 12:1687892-1688794 |
| 12:1828260-1829463 | 12:1863523-1866195 | 12:2228683-2230899 |
| 12:2383862-2384764 | 12:2395911-2396820 | 12:2651191-2654215 |
| 12:2771164-2772130 | 12:2798368-2799353 | 12:2824443-2825398 |
| 12:2994348-2995331 | 12:3318027-3319191 | 12:3367436-3368417 |
| 12:3724280-3725182 | 12:4433607-4434509 | 12:4911378-4913200 |
| 12:5032518-5034333 | 12:5043341-5046684 | 12:5172387-5173321 |
| 12:5492770-5495367 | 12:6018387-6019351 | 12:6046068-6047036 |
| 12:6269632-6270629 | 12:6328203-6329105 | 12:6329389-6330354 |
| 12:6355111-6356013 | 12:6477139-6478106 | 12:6556523-6557508 |
| 12:6672086-6673064 | 12:6770957-6774468 | 12:6967423-6968335 |
| 12:7070337-7071239 | 12:7504349-7505281 | 12:7714961-7715877 |
| 12:8062998-8063900 | 12:8218665-8219613 | 12:8224368-8225332 |
| 12:10998380-10999474 | 12:12475765-12476750 | 12:12716643-12717586 |
| 12:12926807-12928020 | 12:12941814-12942776 | 12:13562465-13564546 |
| 12:19107513-19108501 | 12:20551939-20553382 | 12:24997017-24998143 |
| 12:29515161-29516063 | 12:34210386-34211362 | 12:38162786-38163776 |
| 12:41573013-41574423 | 12:42446208-42447111 | 12:45425002-45425936 |
| 12:45925756-45927624 | 12:47235773-47236931 | 12:47277078-47277983 |
| 12:48142340-48143242 | 12:49026156-49027100 | 12:49791106-49792009 |
| 12:51197402-51198908 | 12:51900309-51901272 | 12:52231582-52232533 |
| 12:52405664-52407316 | 12:52674647-52676600 | 12:52848406-52850233 |
| 12:54030828-54031809 | 12:54181282-54184282 | 12:54611119-54612021 |
| 12:55636629-55637869 | 12:56107139-56108041 | 12:56255025-56256004 |
| 12:56590906-56591843 | 12:56992782-56995792 | 12:57190400-57191383 |
| 12:57674326-57675497 | 12:57824368-57825288 | 12:63002400-63004059 |
| 12:63150009-63150967 | 12:64388584-64391022 | 12:66494817-66496900 |
| 12:68246034-68246936 | 12:68252784-68254261 | 12:68804714-68805696 |
| 12:69610311-69612324 | 12:75305087-75306387 | 12:76072646-76073548 |
| 12:78804151-78805070 | 12:89072255-89073157 | 12:89075930-89076832 |
| 12:90953577-90955881 | 12:91974541-91975443 | 12:94577112-94578014 |
| 12:96234410-96235391 | 12:96246447-96247411 | 12:98454455-98456298 |
| 12:101738388- | 12:104677237- | 12:104756412- |
| 101740557 | 104678139 | 104758447 |
| 12:104928047- | 12:105538575- | 12:106239136- |
| 104929022 | 105539552 | 106240688 |
| 12:106509148- | 12:106519145- | 12:107633377- |
| 106510076 | 106520047 | 107635294 |
| 12:108518346- | 12:108590718- | 12:108792588- |
| 108519803 | 108592501 | 108793490 |
| 12:108811162- | 12:109021656- | 12:109240629- |
| 108812151 | 109022945 | 109241564 |
| 12:109289896- | 12:109439831- | 12:109446717- |
| 109290849 | 109441374 | 109447644 |
| 12:109532816- | 12:109587548- | 12:110105896- |
| 109533779 | 109589505 | 110106886 |
| 12:110536342- | 12:111097412- | 12:111226256- |
| 110537292 | 111100587 | 111227512 |
| 12:111287135- | 12:111319468- | 12:111417568- |
| 111288260 | 111321611 | 111418472 |
| 12:112183272- | 12:113162641- | 12:113231645- |
| 112185210 | 113163637 | 113232549 |
| 12:113265662- | 12:113296005- | 12:113795757- |
| 113266645 | 113296939 | 113796721 |
| 12:113798183- | 12:116969529- | 12:117140978- |
| 113799145 | 116970931 | 117143739 |
| 12:117330410- | 12:119397841- | 12:119713779- |
| 117331711 | 119398812 | 119714761 |
| 12:119718021- | 12:120098458- | 12:120102554- |
| 119718971 | 120099679 | 120104461 |
| 12:120213392- | 12:120303311- | 12:120654074- |
| 120216078 | 120304271 | 120654976 |
| 12:120709401- | 12:120736660- | 12:120738989- |
| 120710316 | 120738312 | 120739985 |
| 12:120836361- | 12:120903429- | 12:121249991- |
| 120837293 | 120905768 | 121251152 |
| 12:121455314- | 12:121509179- | 12:121640967- |
| 121456302 | 121510165 | 121641950 |
| 12:121822643- | 12:121830279- | 12:122516776- |
| 121824470 | 121831919 | 122517738 |
| 12:122704148- | 12:122729963- | 12:122856639- |
| 122705234 | 122731673 | 122857619 |
| 12:122896158- | 12:122929250- | 12:122959096- |
| 122897100 | 122930626 | 122960410 |
| 12:123272322- | 12:123471992- | 12:123785270- |
| 123274055 | 123473423 | 123786402 |
| 12:123787175- | 12:123858648- | 12:123924631- |
| 123789031 | 123860222 | 123925795 |
| 12:123936494- | 12:124012034- | 12:124313718- |
| 123938204 | 124013688 | 124314715 |
| 12:124323913- | 12:124336109- | 12:124347336- |
| 124324894 | 124337078 | 124348730 |
| 12:124348561- | 12:124373614- | 12:124395176- |
| 124349481 | 124375588 | 124396152 |
| 12:124408421- | 12:124416193- | 12:124426222- |
| 124409651 | 124417891 | 124428062 |
| 12:124494796- | 12:124534982- | 12:124786241- |
| 124495785 | 124536474 | 124787176 |
| 12:124807416- | 12:124817184- | 12:124975160- |
| 124808593 | 124818155 | 124976118 |
| 12:125081125- | 12:125114530- | 12:125325615- |
| 125082040 | 125115518 | 125327130 |
| 12:126480398- | 12:127760522- | 12:128800348- |
| 126481323 | 127761741 | 128801286 |
| 12:128819940- | 12:129699525- | 12:130441547- |
| 128820842 | 129701961 | 130442533 |
| 12:130454659- | 12:130483305- | 12:130787187- |
| 130456046 | 130484280 | 130789226 |
| 12:130818422- | 12:130916196- | 12:131043225- |
| 130820355 | 130918954 | 131045885 |
| 12:131124220- | 12:131161876- | 12:131377875- |
| 131125828 | 131164879 | 131378849 |
| 12:131736758- | 12:131752132- | 12:131784503- |
| 131737702 | 131753785 | 131786741 |
| 12:131798773- | 12:131839632- | 12:131849406- |
| 131799706 | 131841311 | 131850344 |
| 12:131863564- | 12:131915368- | 12:131918771- |
| 131864524 | 131916920 | 131919760 |
| 12:131941179- | 12:131944215- | 12:131989731- |
| 131942572 | 131945644 | 131990725 |
| 12:132013777- | 12:132031532- | 12:132035742- |
| 132014704 | 132032495 | 132036738 |
| 12:132044368- | 12:132076883- | 12:132140989- |
| 132046568 | 132078492 | 132142189 |
| 12:132145790- | 12:132200346- | 12:132249579- |
| 132146715 | 132201584 | 132252076 |
| 12:132258149- | 12:132263369- | 12:132318227- |
| 132260094 | 132266502 | 132321327 |
| 12:132321932- | 12:132336948- | 12:132340176- |
| 132326515 | 132337933 | 132343268 |
| 12:132357998- | 12:132359491- | 12:132395119- |
| 132358960 | 132360447 | 132396805 |
| 12:132404635- | 12:132437751- | 12:132440985- |
| 132408218 | 132440691 | 132445900 |
| 12:132450952- | 12:132507768- | 12:132548321- |
| 132452807 | 132508756 | 132551956 |
| 12:132566752- | 12:132583372- | 12:132620699- |
| 132567672 | 132585983 | 132621678 |
| 12:132671817- | 12:132706535- | 12:132718196- |
| 132674750 | 132707522 | 132719456 |
| 12:132720669- | 12:132722636- | 12:132747582- |
| 132721620 | 132723941 | 132748576 |
| 12:132779830- | 12:132793342- | 12:132795477- |
| 132781467 | 132794323 | 132796452 |
| 12:132800599- | 12:132832700- | 12:132840202- |
| 132801683 | 132833627 | 132841816 |
| 12:132850940- | 12:132853865- | 12:132857192- |
| 132851842 | 132855028 | 132858121 |
| 13:20191500-20192625 | 13:20222720-20223708 | 13:20700287-20701189 |
| 13:20903185-20904116 | 13:21103043-21104959 | 13:21701242-21702220 |
| 13:22925247-22927778 | 13:23456022-23456924 | 13:24222860-24223799 |
| 13:24383722-24384791 | 13:25096809-25097769 | 13:26050407-26051384 |
| 13:27928687-27929678 | 13:28056023-28056925 | 13:29038844-29039843 |
| 13:29521094-29522261 | 13:29526427-29527483 | 13:29571809-29572788 |
| 13:30364091-30364993 | 13:30731624-30732584 | 13:31952206-31953135 |
| 13:32310837-32312631 | 13:36920947-36922257 | 13:40480524-40482699 |
| 13:41963217-41964154 | 13:42036413-42037332 | 13:43970308-43971939 |
| 13:45390755-45391954 | 13:45712361-45714925 | 13:49221701-49222795 |
| 13:49305083-49306070 | 13:49839835-49840761 | 13:51251249-51252229 |
| 13:52023813-52024715 | 13:52848191-52849179 | 13:72754807-72756443 |
| 13:74552293-74553227 | 13:75857422-75858385 | 13:95432536-95434391 |
| 13:95587091-95588041 | 13:95931083-95932041 | 13:98176752-98177754 |
| 13:98442186-98443160 | 13:98456933-98457849 | 13:98476494-98477411 |
| 13:98859696-98860619 | 13:99602101-99603003 | 13:99955647-99956622 |
| 13:100439178- | 13:100513273- | 13:100526137- |
| 100440131 | 100514175 | 100527325 |
| 13:100529821- | 13:100642923- | 13:109126798- |
| 100530729 | 100643874 | 109128086 |
| 13:109159612- | 13:109744766- | 13:110199666- |
| 109160514 | 109746419 | 110200772 |
| 13:110460563- | 13:110511900- | 13:110624715- |
| 110461501 | 110513623 | 110625705 |
| 13:110628492- | 13:110637950- | 13:110641704- |
| 110629431 | 110638940 | 110642653 |
| 13:110718626- | 13:111029243- | 13:111275247- |
| 110719798 | 111030549 | 111276149 |
| 13:111318884- | 13:111324578- | 13:111328248- |
| 111319798 | 111326543 | 111329215 |
| 13:111508149- | 13:111538244- | 13:111562212- |
| 111509470 | 111539450 | 111563146 |
| 13:111585775- | 13:111621967- | 13:111667950- |
| 111587505 | 111624549 | 111669993 |
| 13:111969902- | 13:112035718- | 13:112191961- |
| 111970952 | 112038601 | 112192897 |
| 13:112228058- | 13:112449796- | 13:112545917- |
| 112229057 | 112453574 | 112546819 |
| 13:112636671- | 13:112645332- | 13:112727834- |
| 112639147 | 112646246 | 112728779 |
| 13:112741271- | 13:112750621- | 13:112756271- |
| 112742173 | 112751614 | 112757212 |
| 13:112762278- | 13:112764874- | 13:112774340- |
| 112763251 | 112766605 | 112776627 |
| 13:112779843- | 13:112785121- | 13:112807217- |
| 112784017 | 112786042 | 112808150 |
| 13:112831887- | 13:112842459- | 13:112845313- |
| 112834681 | 112844832 | 112846890 |
| 13:112857545- | 13:112870621- | 13:112881270- |
| 112859632 | 112871909 | 112882222 |
| 13:112882682- | 13:112889470- | 13:112890749- |
| 112884328 | 112890420 | 112891808 |
| 13:112910272- | 13:112967206- | 13:112976930- |
| 112911416 | 112969535 | 112980906 |
| 13:112986045- | 13:113004786- | 13:113033323- |
| 112987520 | 113006700 | 113034269 |
| 13:113045610- | 13:113062525- | 13:113074582- |
| 113046662 | 113066091 | 113075576 |
| 13:113078037- | 13:113080765- | 13:113087877- |
| 113079007 | 113081676 | 113088820 |
| 13:113088717- | 13:113121264- | 13:113142989- |
| 113090986 | 113122903 | 113144566 |
| 13:113148052- | 13:113170957- | 13:113274229- |
| 113149809 | 113172393 | 113275131 |
| 13:113321128- | 13:113400075- | 13:113416439- |
| 113322621 | 113401047 | 113417624 |
| 13:113432557- | 13:113441298- | 13:113448603- |
| 113434302 | 113442475 | 113449559 |
| 13:113453019- | 13:113459468- | 13:113468408- |
| 113456162 | 113461411 | 113469334 |
| 13:113510541- | 13:113543514- | 13:113546720- |
| 113511510 | 113544915 | 113548767 |
| 13:113609639- | 13:113629174- | 13:113633104- |
| 113610561 | 113630531 | 113634053 |
| 13:113635830- | 13:113655816- | 13:113669221- |
| 113636762 | 113659644 | 113672061 |
| 13:113974574- | 13:114001934- | 13:114026203- |
| 113975544 | 114002917 | 114028230 |
| 13:114080629- | 13:114095514- | 13:114120750- |
| 114081604 | 114098190 | 114122366 |
| 13:114139619- | 14:20434757-20436612 | 14:20835542-20836444 |
| 114141082 | | |
| 14:21028474-21031275 | 14:21090154-21092413 | 14:21287104-21288523 |
| 14:22819275-22823784 | 14:22845323-22847680 | 14:23388130-23389067 |
| 14:23417571-23418530 | 14:23934247-23936069 | 14:24146161-24147141 |
| 14:24365011-24370124 | 14:31014811-31015713 | 14:31030285-31031187 |
| 14:36666143-36667105 | 14:37597866-37598859 | 14:38208027-38209858 |
| 14:49927420-49928323 | 14:50704217-50705747 | 14:50744696-50745598 |
| 14:51751697-51753675 | 14:51967483-51968426 | 14:52398893-52399851 |
| 14:56273645-56274569 | 14:58662896-58663838 | 14:58827932-58829053 |
| 14:59476731-59477633 | 14:62056519-62057505 | 14:63594611-63595513 |
| 14:63761415-63762318 | 14:64215404-64216736 | 14:64748738-64749736 |
| 14:65279555-65280544 | 14:67519117-67520019 | 14:67600505-67601476 |
| 14:68669387-68670289 | 14:68702812-68703714 | 14:68911733-68912688 |
| 14:69054775-69055730 | 14:69661762-69662664 | 14:69722397-69723299 |
| 14:70030440-70031342 | 14:70555655-70556977 | 14:72679916-72680818 |
| 14:73589440-73590435 | 14:73777025-73777949 | 14:74260222-74261204 |
| 14:74954914-74956352 | 14:75409425-75410333 | 14:75539059-75539961 |
| 14:75546037-75547410 | 14:75979154-75982397 | 14:76083382-76084297 |
| 14:76883133-76884102 | 14:77912105-77913007 | 14:78172899-78174409 |
| 14:85620929-85624521 | 14:89162005-89164176 | 14:90521808-90524288 |
| 14:90540845-90541812 | 14:90570160-90571091 | 14:90575579-90576557 |
| 14:91291585-91293103 | 14:91338816-91340454 | 14:91419761-91420754 |
| 14:91482838-91483740 | 14:92687862-92688860 | 14:93429533-93430526 |
| 14:93654285-93655187 | 14:94561043-94561945 | 14:95410208-95411110 |
| 14:95441120-95442055 | 14:95449350-95450295 | 14:96249094-96250035 |
| 14:96662594-96663775 | 14:96900323-96901225 | 14:96964237-96965479 |
| 14:97546101-97547256 | 14:99172852-99175227 | 14:99319375-99321565 |
| 14:99347297-99349280 | 14:99382011-99384262 | 14:99413690-99414666 |
| 14:99424832-99425734 | 14:99521891-99522859 | 14:99801379-99802346 |
| 14:100148717- | 14:100445180- | 14:100476123- |
| 100149806 | 100446082 | 100477111 |
| 14:100692588- | 14:100705343- | 14:100733830- |
| 100693580 | 100706297 | 100735847 |
| 14:100835632- | 14:100856703- | 14:100867711- |
| 100836576 | 100857609 | 100869564 |
| 14:100873239- | 14:100879357- | 14:100892947- |
| 100875725 | 100886082 | 100895560 |
| 14:100938812- | 14:101030721- | 14:101038647- |
| 100939956 | 101034318 | 101044782 |
| 14:101059151- | 14:101063749- | 14:101227062- |
| 101061317 | 101066546 | 101228035 |
| 14:101475322- | 14:101520151- | 14:101586372- |
| 101476228 | 101521089 | 101588753 |
| 14:101763352- | 14:101890018- | 14:101924545- |
| 101764254 | 101890920 | 101925495 |
| 14:101955269- | 14:102033412- | 14:102208304- |
| 101956790 | 102034371 | 102209278 |
| 14:102217144- | 14:102443384- | 14:102507361- |
| 102218342 | 102444327 | 102509612 |
| 14:102776118- | 14:102869888- | 14:102900154- |
| 102778015 | 102870840 | 102901603 |
| 14:102905042- | 14:102906217- | 14:102910034- |
| 102906041 | 102909283 | 102911025 |
| 14:102943294- | 14:102963450- | 14:102965869- |
| 102947190 | 102964873 | 102967511 |
| 14:102999079- | 14:103002560- | 14:103109941- |
| 103000023 | 103003966 | 103110918 |
| 14:103125598- | 14:103559989- | 14:103626670- |
| 103128207 | 103561407 | 103627655 |
| 14:103685450- | 14:103712820- | 14:103715957- |
| 103686371 | 103713722 | 103717713 |
| 14:103928612- | 14:104384981- | 14:104389616- |
| 103929582 | 104386944 | 104391051 |
| 14:104574520- | 14:104636165- | 14:104709701- |
| 104575919 | 104637702 | 104710690 |
| 14:104712671- | 14:104894338- | 14:105142591- |
| 104713635 | 104895259 | 105144041 |
| 14:105147665- | 14:105188514- | 14:105246981- |
| 105149657 | 105189506 | 105248802 |
| 14:105281066- | 14:105329390- | 14:105383337- |
| 105282050 | 105331172 | 105384406 |
| 14:105392247- | 14:105437885- | 14:105441704- |
| 105393235 | 105438787 | 105443265 |
| 14:105467029- | 14:105496744- | 14:105604234- |
| 105467958 | 105499359 | 105605737 |
| 14:105854474- | 15:22787657-22788650 | 15:22869558-22870522 |
| 105856196 | | |
| 15:22903196-22904995 | 15:22911989-22912966 | 15:22915548-22919313 |
| 15:22925495-22926703 | 15:23644952-23646592 | 15:24674543-24677007 |
| 15:27803522-27807094 | 15:28002929-28004902 | 15:28026251-28027853 |
| 15:28111107-28113121 | 15:28114314-28115894 | 15:28116727-28117690 |
| 15:29102779-29104776 | 15:29135942-29136885 | 15:29611207-29612863 |
| 15:31371143-31372787 | 15:31452813-31454227 | 15:32671978-32672921 |
| 15:34238649-34239551 | 15:35091996-35093351 | 15:35298157-35300019 |
| 15:36818082-36819053 | 15:39820442-39821383 | 15:40149133-40150064 |
| 15:41485927-41487237 | 15:41838001-41839985 | 15:42035756-42037326 |
| 15:42358013-42359339 | 15:42735100-42736059 | 15:42738078-42738980 |
| 15:43799421-43800407 | 15:43873345-43874258 | 15:48925373-48926274 |
| 15:48961671-48962844 | 15:49887698-49888604 | 15:51625474-51626388 |
| 15:52176104-52177055 | 15:52294332-52297039 | 15:52788779-52789727 |
| 15:57731508-57732410 | 15:59170858-59172483 | 15:59440845-59441771 |
| 15:60394786-60399294 | 15:62223082-62225256 | 15:62644455-62646596 |
| 15:62769883-62770850 | 15:62822615-62824766 | 15:62953062-62954046 |
| 15:63068204-63069106 | 15:65265259-65266176 | 15:65632673-65633575 |
| 15:66039122-66040968 | 15:66780760-66782008 | 15:67193801-67194746 |
| 15:68301732-68302634 | 15:68990432-68991374 | 15:69313531-69314893 |
| 15:70253829-70254939 | 15:72196529-72199142 | 15:72737087-72737989 |
| 15:73211591-73212493 | 15:73596696-73597664 | 15:74234299-74236926 |
| 15:74365045-74368676 | 15:74421615-74422551 | 15:74798094-74799047 |
| 15:74815942-74817053 | 15:75355742-75357568 | 15:75648744-75649646 |
| 15:75687403-75688392 | 15:77055613-77056531 | 15:77071726-77072999 |
| 15:77284610-77285527 | 15:77896869-77897845 | 15:77907600-77908582 |
| 15:78079286-78080218 | 15:78762720-78763659 | 15:79178599-79179790 |
| 15:79558283-79561121 | 15:80331490-80332658 | 15:80754110-80755012 |
| 15:80780800-80782081 | 15:81195376-81197305 | 15:81332221-81333129 |
| 15:84973803-84974705 | 15:85675650-85677523 | 15:86557731-86558647 |
| 15:88603677-88604653 | 15:89366000-89369455 | 15:89377070-89380169 |
| 15:89627875-89628833 | 15:89648745-89649982 | 15:89742866-89743814 |
| 15:89747989-89750579 | 15:90360291-90361912 | 15:90866768-90869392 |
| 15:90879649-90880634 | 15:91024424-91025326 | 15:92579165-92581593 |
| 15:92813147-92814049 | 15:93072808-93073799 | 15:93432046-93433004 |
| 15:94354196-94355640 | 15:96353208-96354151 | 15:98707086-98708774 |
| 15:98766253-98767176 | 15:98953371-98954297 | 15:98960560-98961533 |
| 15:99005825-99007197 | 15:99099767-99100670 | 15:99799439-99800434 |
| 15:99973793-99974714 | 15:100291253- | 15:101046069- |
| | 100293618 | 101048254 |
| 15:101156236- | 15:101157771- | 15:101177933- |
| 101157539 | 101159603 | 101179832 |
| 15:101306452- | 15:101496240- | 15:101570710- |
| 101307353 | 101498077 | 101571834 |
| 16:62290-63614 | 16:290640-291632 | 16:301320-302938 |
| 16:317510-319294 | 16:360380-361902 | 16:373770-374740 |
| 16:377023-378234 | 16:385363-386364 | 16:409269-410907 |
| 16:420571-421473 | 16:473268-474929 | 16:481842-483714 |
| 16:520544-521508 | 16:537280-538267 | 16:538031-539009 |
| 16:579610-580592 | 16:596094-597024 | 16:634128-635058 |
| 16:656109-657053 | 16:658045-659013 | 16:666879-668031 |
| 16:672011-673449 | 16:682093-683079 | 16:686300-687267 |
| 16:727807-730139 | 16:758954-761829 | 16:765524-767546 |
| 16:791990-792976 | 16:793948-796623 | 16:832216-834384 |
| 16:840731-845852 | 16:846288-849657 | 16:850474-851463 |
| 16:873121-874163 | 16:875289-876906 | 16:877605-880588 |
| 16:887510-889228 | 16:891399-892380 | 16:909200-912630 |
| 16:913443-914431 | 16:915272-922907 | 16:927339-928254 |
| 16:930861-934725 | 16:938433-940763 | 16:951106-952099 |
| 16:954539-956713 | 16:1038689-1039633 | 16:1060455-1062872 |
| 16:1077561-1080768 | 16:1127900-1130110 | 16:1171308-1173915 |
| 16:1200069-1201060 | 16:1207257-1211862 | 16:1255816-1256782 |
| 16:1263004-1263906 | 16:1315071-1316006 | 16:1317078-1317980 |
| 16:1345804-1346774 | 16:1373770-1375805 | 16:1401020-1402161 |
| 16:1450616-1451537 | 16:1451626-1452583 | 16:1464568-1465510 |
| 16:1485535-1487563 | 16:1496238-1497913 | 16:1503109-1504083 |
| 16:1520380-1521314 | 16:1529510-1530500 | 16:1557308-1560831 |
| 16:1564386-1565533 | 16:1625361-1627586 | 16:1671550-1672528 |
| 16:1672441-1673437 | 16:1680026-1681069 | 16:1732007-1733825 |
| 16:1742754-1744022 | 16:1748090-1749068 | 16:1766683-1768821 |
| 16:1775353-1776326 | 16:1821564-1822504 | 16:1991551-1992543 |
| 16:2076393-2078191 | 16:2079216-2080201 | 16:2082855-2085187 |
| 16:2084955-2085880 | 16:2085750-2087514 | 16:2091293-2092204 |
| 16:2095116-2096093 | 16:2184477-2185456 | 16:2235226-2239270 |
| 16:2283711-2285867 | 16:2323994-2324935 | 16:2499946-2500940 |
| 16:2595394-2596296 | 16:2689818-2691299 | 16:2755825-2757028 |
| 16:2761512-2762498 | 16:2765841-2767244 | 16:2828862-2830909 |
| 16:2970586-2972351 | 16:2999383-3000285 | 16:3262436-3263338 |
| 16:3389276-3390178 | 16:3482392-3483363 | 16:3562604-3565144 |
| 16:3673864-3674826 | 16:3724992-3726997 | 16:3745363-3746292 |
| 16:4207357-4208878 | 16:4334798-4335709 | 16:4614520-4615500 |
| 16:4634243-4635153 | 16:4667228-4668202 | 16:4685705-4687387 |
| 16:4752440-4753430 | 16:4797659-4798612 | 16:4880204-4881106 |
| 16:6483214-6484318 | 16:8847343-8850117 | 16:8894301-8895203 |
| 16:9762818-9764489 | 16:10532489-10533476 | 16:10707953-10708889 |
| 16:10906631-10908533 | 16:11121036-11121938 | 16:11125580-11127054 |
| 16:11275225-11277983 | 16:11280925-11281960 | 16:11675288-11676250 |
| 16:12096882-12099095 | 16:12184066-12184968 | 16:12513976-12515742 |
| 16:12523813-12525174 | 16:12704205-12705169 | 16:12802502-12805178 |
| 16:15616888-15617841 | 16:15724467-15725493 | 16:16364362-16365302 |
| 16:19114372-19115369 | 16:19115259-19116516 | 16:20690599-20692001 |
| 16:21557606-21558541 | 16:24219680-24220582 | 16:27218442-27219403 |
| 16:27226134-27227121 | 16:27488478-27489452 | 16:27699346-27700265 |
| 16:27702821-27703781 | 16:27749392-27750675 | 16:29159295-29160227 |
| 16:29193591-29194891 | 16:29288812-29289793 | 16:29841404-29843349 |
| 16:30397766-30398752 | 16:30833950-30834945 | 16:30929659-30930629 |
| 16:30984093-30985017 | 16:31079836-31080829 | 16:31142777-31143726 |
| 16:46970940-46971842 | 16:48495660-48497399 | 16:52935450-52936403 |
| 16:54287887-54288838 | 16:56193237-56194215 | 16:56257396-56258298 |
| 16:56363647-56364564 | 16:57381414-57383477 | 16:57733088-57735862 |
| 16:57970572-57971527 | 16:65919361-65920347 | 16:66565940-66567214 |
| 16:66800121-66801023 | 16:67003428-67004332 | 16:67281755-67282657 |
| 16:67429976-67432784 | 16:67661068-67663643 | 16:67674997-67675983 |
| 16:67908614-67909568 | 16:67963463-67968000 | 16:68020979-68021913 |
| 16:68370376-68373047 | 16:68446595-68448584 | 16:68697269-68699122 |
| 16:68907817-68908719 | 16:69109321-69110300 | 16:69110467-69111387 |
| 16:69422826-69423761 | 16:69748362-69749312 | 16:69904602-69905521 |
| 16:69906876-69907813 | 16:69930841-69933811 | 16:69934724-69936231 |
| 16:69940713-69942794 | 16:70294220-70295133 | 16:70469996-70471786 |
| 16:70472673-70473642 | 16:71535749-71538211 | 16:71640370-71641316 |
| 16:72006987-72007889 | 16:72172186-72173925 | 16:73091223-73092672 |
| 16:75114664-75116144 | 16:75206121-75208743 | 16:75494095-75496529 |
| 16:75555148-75556114 | 16:75647167-75648152 | 16:81498996-81499989 |
| 16:81682168-81683411 | 16:81691745-81693993 | 16:81935732-81937699 |
| 16:82169460-82171866 | 16:83829457-83831167 | 16:83914672-83916069 |
| 16:84051857-84054949 | 16:84058815-84060191 | 16:84318794-84319781 |
| 16:84451579-84452559 | 16:84458478-84459405 | 16:84656376-84657496 |
| 16:84661276-84663348 | 16:84763371-84764365 | 16:84883336-84884238 |
| 16:84906922-84908227 | 16:84941565-84942544 | 16:84964413-84965366 |
| 16:84974239-84976594 | 16:85098253-85100143 | 16:85207762-85209478 |
| 16:85281063-85283515 | 16:85373194-85374435 | 16:85429846-85432581 |
| 16:85508639-85510620 | 16:85635438-85637714 | 16:85654022-85655373 |
| 16:85655679-85656618 | 16:85843061-85844090 | 16:86375132-86376034 |
| 16:86580700-86581616 | 16:86604767-86605692 | 16:86916673-86918646 |
| 16:87056196-87058573 | 16:87140500-87141443 | 16:87345711-87347692 |
| 16:87408844-87409839 | 16:87413643-87414584 | 16:87416850-87418832 |
| 16:87431719-87432621 | 16:87465785-87466881 | 16:87477346-87478730 |
| 16:87543118-87544599 | 16:87636273-87638090 | 16:87685033-87686173 |
| 16:87695916-87698523 | 16:87699569-87700533 | 16:87700676-87703860 |
| 16:87708153-87710224 | 16:87725253-87727936 | 16:87735865-87736807 |
| 16:87744785-87745743 | 16:87821929-87824135 | 16:87955940-87957622 |
| 16:88065556-88068328 | 16:88070940-88073166 | 16:88076140-88078450 |
| 16:88128755-88131512 | 16:88131504-88133787 | 16:88224137-88225128 |
| 16:88227279-88228231 | 16:88260415-88261381 | 16:88321636-88323192 |
| 16:88352808-88355164 | 16:88434782-88439198 | 16:88470301-88472477 |
| 16:88483420-88485621 | 16:88566775-88567709 | 16:88616991-88617930 |
| 16:88644634-88647252 | 16:88653497-88656797 | 16:88693370-88695454 |
| 16:88714516-88715484 | 16:88716103-88717783 | 16:88720706-88721627 |
| 16:88722139-88723134 | 16:88737488-88739113 | 16:88739603-88742071 |
| 16:88815618-88819167 | 16:88826698-88829249 | 16:88834694-88835793 |
| 16:88837521-88839872 | 16:88841666-88842648 | 16:88866117-88869996 |
| 16:88873984-88877771 | 16:88884476-88885768 | 16:88897580-88902376 |
| 16:88902626-88904830 | 16:88918968-88920405 | 16:88926102-88927784 |
| 16:88933236-88935078 | 16:88967111-88972388 | 16:89041435-89044085 |
| 16:89070800-89071767 | 16:89095444-89097935 | 16:89102330-89103310 |
| 16:89106899-89107893 | 16:89110858-89112722 | 16:89118378-89120593 |
| 16:89136500-89137481 | 16:89197952-89198915 | 16:89270392-89272175 |
| 16:89288047-89289020 | 16:89290177-89291727 | 16:89338824-89340387 |
| 16:89395404-89396917 | 16:89523405-89524364 | 16:89548158-89549157 |
| 16:89551877-89552873 | 16:89554909-89556038 | 16:89583559-89585122 |
| 16:89594848-89595764 | 16:89605025-89606011 | 16:89636077-89637039 |
| 16:89672753-89673781 | 16:89821893-89822825 | 16:89854961-89856728 |
| 16:89860636-89861614 | 16:89895104-89896198 | 16:89906239-89907185 |
| 16:89915173-89916742 | 16:89933399-89934348 | 17:206415-207378 |
| 17:262976-264818 | 17:273087-275460 | 17:284996-285988 |
| 17:286232-287156 | 17:288980-291268 | 17:327149-328051 |
| 17:357136-359452 | 17:441201-443700 | 17:445425-447232 |
| 17:519528-520486 | 17:791336-792303 | 17:840269-841193 |
| 17:841022-842016 | 17:946206-947710 | 17:958521-959594 |
| 17:1011953-1013558 | 17:1024938-1026955 | 17:1094724-1095805 |
| 17:1124790-1126827 | 17:1160198-1162441 | 17:1225538-1226461 |
| 17:1437061-1437962 | 17:1471472-1472451 | 17:1574052-1576276 |
| 17:1674760-1675749 | 17:1727115-1728822 | 17:1729948-1730895 |
| 17:1733245-1734211 | 17:1883205-1884135 | 17:1918130-1919095 |
| 17:1936153-1937789 | 17:1985076-1985993 | 17:2243507-2244409 |
| 17:2330804-2331714 | 17:2413890-2414837 | 17:2696009-2696974 |
| 17:3471676-3473146 | 17:3654971-3655951 | 17:3659804-3660757 |
| 17:3812627-3813678 | 17:3873260-3874215 | 17:3888071-3889309 |
| 17:3897008-3897962 | 17:3936956-3938081 | 17:3940585-3941716 |
| 17:3946512-3948638 | 17:4008258-4009364 | 17:4164650-4165590 |
| 17:4276761-4277663 | 17:4705240-4706284 | 17:4738208-4741504 |
| 17:4787626-4790040 | 17:6537592-6538513 | 17:6653949-6655678 |
| 17:6852058-6854023 | 17:7107370-7108350 | 17:7173076-7174936 |
| 17:7321161-7322117 | 17:7351592-7353894 | 17:7380003-7380911 |
| 17:7406577-7408805 | 17:7418321-7419847 | 17:7424750-7426929 |
| 17:7556626-7558841 | 17:7588932-7590875 | 17:7700949-7702377 |
| 17:7752703-7754659 | 17:7766851-7768331 | 17:7993847-7994786 |
| 17:8391878-8392853 | 17:8557087-8558029 | 17:8827897-8829262 |
| 17:8910958-8913049 | 17:9904076-9905958 | 17:10051039-10051956 |
| 17:10629540-10630442 | 17:10657912-10658888 | 17:14205809-14206789 |
| 17:15437606-15438516 | 17:15682320-15683294 | 17:16348848-16349750 |
| 17:17163817-17165402 | 17:17202003-17204069 | 17:17206075-17207071 |
| 17:17216426-17217555 | 17:17223416-17224399 | 17:17382992-17384921 |
| 17:17709466-17710412 | 17:17796355-17798916 | 17:17805429-17806335 |
| 17:17813384-17815747 | 17:17993539-17994441 | 17:18041129-18042056 |
| 17:18083768-18084670 | 17:18118660-18120922 | 17:18244452-18245354 |
| 17:18250882-18251847 | 17:18263754-18264732 | 17:19331235-19332137 |
| 17:19446289-19447288 | 17:19583530-19584432 | 17:19716112-19717768 |
| 17:19868317-19869357 | 17:20612072-20613403 | 17:20868022-20868969 |
| 17:20936952-20939165 | 17:21001921-21003003 | 17:21374776-21375963 |
| 17:21414745-21415724 | 17:27157599-27159009 | 17:27418150-27419945 |
| 17:27595337-27596239 | 17:27630293-27631728 | 17:28246060-28247824 |
| 17:28400173-28401075 | 17:29166042-29167019 | 17:29208674-29209631 |
| 17:29620790-29622235 | 17:30333651-30335574 | 17:31574302-31575843 |
| 17:31709025-31710236 | 17:33533581-33535010 | 17:35765674-35767886 |
| 17:35769546-35770495 | 17:35939618-35940556 | 17:37056541-37057492 |
| 17:37512096-37513718 | 17:39636007-39637477 | 17:39765277-39766711 |
| 17:40013828-40015131 | 17:41008216-41009896 | 17:41097043-41098638 |
| 17:41350279-41351468 | 17:41518395-41520371 | 17:41586636-41587624 |
| 17:41818783-41819764 | 17:41853204-41854420 | 17:42040186-42041088 |
| 17:42404570-42406022 | 17:42547263-42549355 | 17:42573413-42574358 |
| 17:42686303-42687841 | 17:43362606-43363999 | 17:43378674-43379618 |
| 17:43719838-43722170 | 17:43941775-43944000 | 17:44067218-44068385 |
| 17:44086534-44087514 | 17:44211813-44213502 | 17:45393154-45394264 |
| 17:45395591-45397549 | 17:45404846-45406876 | 17:45997620-45998579 |
| 17:46721677-46722671 | 17:46773223-46774125 | 17:48482770-48483686 |
| 17:48738442-48739387 | 17:49505752-49507105 | 17:49513604-49515813 |
| 17:50105500-50106472 | 17:50167193-50168145 | 17:50264326-50265315 |
| 17:50471980-50472907 | 17:50536526-50537523 | 17:50833271-50836591 |
| 17:51164682-51167887 | 17:53821947-53824067 | 17:56510375-56512212 |
| 17:56752818-56753720 | 17:58328037-58329480 | 17:58543584-58545126 |
| 17:61379501-61380473 | 17:61415057-61415981 | 17:61486760-61487725 |
| 17:62807342-62809374 | 17:63437828-63438745 | 17:63496423-63497834 |
| 17:63578326-63579310 | 17:63881388-63882291 | 17:63931727-63934156 |
| 17:65538023-65538994 | 17:65557008-65557911 | 17:66447853-66448833 |
| 17:66525998-66526934 | 17:66675536-66676521 | 17:67029942-67031717 |
| 17:68250064-68251024 | 17:68419666-68421143 | 17:69511109-69512100 |
| 17:69541320-69542222 | 17:69896640-69897542 | 17:73284063-73284994 |
| 17:73285511-73286481 | 17:73422961-73424487 | 17:73552222-73553145 |
| 17:73747781-73749332 | 17:74438368-74439270 | 17:74735822-74738231 |
| 17:74835539-74836487 | 17:74930135-74931261 | 17:75093567-75094542 |
| 17:75493518-75494456 | 17:75501505-75506819 | 17:75518213-75519799 |
| 17:75521439-75522398 | 17:75635180-75636083 | 17:75717998-75718960 |
| 17:75723135-75724580 | 17:75729463-75730895 | 17:75733066-75734029 |
| 17:75890603-75892100 | 17:75914637-75915618 | 17:76006735-76008051 |
| 17:76021887-76022977 | 17:76303861-76304763 | 17:76390549-76392032 |
| 17:76469205-76470456 | 17:76470784-76472010 | 17:76718083-76718985 |
| 17:76777862-76778813 | 17:76914874-76916815 | 17:77212950-77213931 |
| 17:77279465-77282619 | 17:77285435-77288549 | 17:77401381-77403033 |
| 17:77481715-77482701 | 17:77526863-77529806 | 17:77590915-77591825 |
| 17:77954173-77955120 | 17:78104483-78105473 | 17:78225036-78226374 |
| 17:78254947-78255913 | 17:78396746-78397728 | 17:78422809-78423769 |
| 17:78474405-78476721 | 17:78479169-78480153 | 17:78536876-78537842 |
| 17:78560497-78561465 | 17:78570492-78572278 | 17:78575691-78576593 |
| 17:78621336-78623155 | 17:78628932-78629870 | 17:78674842-78675815 |
| 17:78679770-78681574 | 17:78797900-78799734 | 17:78802200-78803187 |
| 17:78899140-78900055 | 17:78971606-78973183 | 17:79079488-79080944 |
| 17:79081692-79083486 | 17:79085842-79087594 | 17:79093982-79094926 |
| 17:79131913-79133072 | 17:79144494-79146607 | 17:79393417-79394405 |
| 17:79461981-79462893 | 17:79734941-79735934 | 17:79784251-79785221 |
| 17:79788209-79795979 | 17:79832064-79834760 | 17:79859206-79861050 |
| 17:79939617-79941591 | 17:79941613-79945039 | 17:79945344-79946287 |
| 17:80099090-80100567 | 17:80112078-80113227 | 17:80180979-80182942 |
| 17:80214554-80215535 | 17:80339298-80340782 | 17:80348956-80349882 |
| 17:80353494-80354455 | 17:80670416-80671363 | 17:80693448-80696117 |
| 17:80745884-80747221 | 17:80794636-80795580 | 17:80802211-80805254 |
| 17:80827516-80828773 | 17:80829973-80831141 | 17:80837245-80839233 |
| 17:80843304-80845730 | 17:80847263-80849597 | 17:80881995-80883751 |
| 17:80895203-80896158 | 17:80903620-80905556 | 17:80905688-80906618 |
| 17:80960824-80961819 | 17:80965335-80967205 | 17:81026251-81028291 |
| 17:81030599-81033284 | 17:81054434-81055399 | 17:81067851-81068776 |
| 17:81077543-81078472 | 17:81083534-81084474 | 17:81125460-81126428 |
| 17:81209617-81210612 | 17:81249961-81252041 | 17:81260648-81262895 |
| 17:81270653-81271601 | 17:81273709-81274679 | 17:81279282-81280279 |
| 17:81416466-81418324 | 17:81539427-81541070 | 17:81547409-81548311 |
| 17:81548875-81549787 | 17:81564874-81565858 | 17:81689833-81690827 |
| 17:81692664-81693658 | 17:81713987-81716669 | 17:81844815-81846762 |
| 17:81934636-81935621 | 17:81940660-81942561 | 17:81947712-81949066 |
| 17:81956180-81957397 | 17:82003114-82005858 | 17:82046352-82047270 |
| 17:82053939-82054922 | 17:82065755-82066749 | 17:82088399-82089373 |
| 17:82154133-82155216 | 17:82249026-82251102 | 17:82326897-82327805 |
| 17:82345106-82346092 | 17:82397532-82398526 | 17:82436021-82437647 |
| 17:82491328-82492310 | 17:82597604-82598521 | 17:82602071-82603292 |
| 17:82626366-82627268 | 17:82649582-82650746 | 17:82714787-82715689 |
| 17:82718513-82719415 | 17:82776489-82777464 | 17:82788819-82789764 |
| 17:82790920-82791895 | 17:82794811-82796727 | 17:82804001-82808218 |
| 17:82831984-82833014 | 17:82835653-82836797 | 17:82839525-82840566 |
| 17:82851246-82853125 | 17:82860272-82861782 | 17:82862372-82864338 |
| 17:82867109-82868095 | 17:82871309-82872282 | 17:82886824-82888774 |
| 17:82899979-82904273 | 17:82905733-82906635 | 17:82919427-82920409 |
| 17:82926500-82927466 | 17:82930793-82931745 | 17:82939883-82941652 |
| 17:82976586-82978401 | 17:82983089-82984104 | 17:83006036-83006964 |
| 17:83007828-83008778 | 17:83017320-83020059 | 17:83020078-83021374 |
| 17:83028525-83030298 | 17:83055682-83058172 | 17:83064807-83067027 |
| 17:83072827-83076796 | 17:83083691-83086448 | 17:83090721-83091623 |
| 17:83093706-83095792 | 18:578702-580982 | 18:2911881-2913582 |
| 18:3011301-3013100 | 18:8658235-8659137 | 18:8662124-8663082 |
| 18:8783815-8784802 | 18:10522594-10523496 | 18:12067520-12068914 |
| 18:12779240-12780216 | 18:13868278-13869923 | 18:14474996-14475982 |
| 18:23531611-23532580 | 18:23872256-23873671 | 18:31101832-31102973 |
| 18:39752474-39753531 | 18:44679718-44680715 | 18:46656359-46658240 |
| 18:46914510-46915943 | 18:46945921-46947602 | 18:47035149-47036656 |
| 18:48028540-48030106 | 18:48040711-48041613 | 18:48858401-48861304 |
| 18:48862014-48862977 | 18:50728653-50730211 | 18:54354457-54355412 |
| 18:57586793-57587725 | 18:58256246-58257209 | 18:59897594-59898534 |
| 18:63475485-63477649 | 18:63586066-63587697 | 18:74314911-74316661 |
| 18:74533293-74534342 | 18:76378190-76379145 | 18:76378896-76379870 |
| 18:76894470-76895421 | 18:76916161-76917115 | 18:78547898-78548828 |
| 18:78725863-78727405 | 18:78796617-78798014 | 18:78994759-78995884 |
| 18:79022223-79023201 | 18:79361320-79362283 | 18:79424904-79427334 |
| 18:79432943-79433865 | 18:79446675-79447652 | 18:79461471-79462445 |
| 18:79474507-79475499 | 18:79479982-79481186 | 18:79487106-79488073 |
| 18:79510616-79511605 | 18:79512038-79513509 | 18:79628661-79629590 |
| 18:79732740-79733731 | 18:79735833-79737676 | 18:79746996-79747990 |
| 18:79782405-79783716 | 18:79806649-79808094 | 18:79884980-79885979 |
| 18:79914845-79915828 | 18:79967482-79968420 | 19:398024-399008 |
| 19:421867-423054 | 19:462975-464870 | 19:506575-507572 |
| 19:542097-543056 | 19:577387-578380 | 19:617524-618443 |
| 19:641320-642277 | 19:735736-736691 | 19:757349-758797 |
| 19:814375-816073 | 19:882887-883883 | 19:939271-940251 |
| 19:1011558-1012535 | 19:1050527-1051685 | 19:1079514-1080429 |
| 19:1108011-1108980 | 19:1116739-1117894 | 19:1140430-1141378 |
| 19:1157139-1158080 | 19:1220084-1221065 | 19:1225989-1227517 |
| 19:1376587-1377556 | 19:1398638-1399551 | 19:1419524-1420484 |
| 19:1486455-1487415 | 19:1631824-1633520 | 19:1645937-1646911 |
| 19:1799389-1800858 | 19:1815433-1816423 | 19:1818123-1819100 |
| 19:1954869-1956308 | 19:1969460-1970440 | 19:2114746-2115726 |
| 19:2136438-2137408 | 19:2199444-2200430 | 19:2216175-2217155 |
| 19:2229730-2230722 | 19:2241647-2242615 | 19:2246201-2247159 |
| 19:2275920-2276919 | 19:2277952-2279392 | 19:2321131-2322441 |
| 19:2340302-2341205 | 19:2351240-2352215 | 19:2443505-2445060 |
| 19:2510566-2512259 | 19:2650211-2651198 | 19:2732896-2733885 |
| 19:2757118-2758104 | 19:2806671-2807573 | 19:3114502-3115479 |
| 19:3120730-3121684 | 19:3121794-3123200 | 19:3200980-3201963 |
| 19:3380891-3382060 | 19:3462701-3464334 | 19:3522464-3523459 |
| 19:3532920-3533837 | 19:3648106-3649134 | 19:3655023-3655986 |
| 19:3658819-3660569 | 19:3669265-3670250 | 19:3675089-3677673 |
| 19:3824842-3825813 | 19:3834346-3835316 | 19:3937936-3939255 |
| 19:3941785-3942750 | 19:4037076-4038769 | 19:4090720-4091690 |
| 19:4102057-4103038 | 19:4361254-4362246 | 19:4675690-4676630 |
| 19:4684390-4685309 | 19:4689011-4690000 | 19:4890463-4891975 |
| 19:4943600-4944936 | 19:4952324-4953255 | 19:4965868-4966770 |
| 19:4987630-4989279 | 19:5009793-5010695 | 19:5047150-5048147 |
| 19:5048575-5049540 | 19:5110055-5111173 | 19:5112156-5113126 |
| 19:5113643-5114640 | 19:5118769-5119769 | 19:5222463-5223457 |
| 19:5587224-5589309 | 19:5684423-5685388 | 19:5692059-5693044 |
| 19:5785266-5786327 | 19:5792134-5793096 | 19:5844008-5844969 |
| 19:5866525-5867515 | 19:6039434-6040382 | 19:6214454-6215428 |
| 19:6707059-6708057 | 19:6711763-6713964 | 19:6729499-6730467 |
| 19:6744357-6745313 | 19:7464507-7465444 | 19:7611299-7612901 |
| 19:7899145-7900131 | 19:7932222-7933375 | 19:7963326-7964301 |
| 19:7964430-7965332 | 19:8403872-8404844 | 19:8485625-8486611 |
| 19:8588959-8590451 | 19:8698538-8699440 | 19:9160695-9161682 |
| 19:9406376-9407375 | 19:9735335-9736300 | 19:9848852-9849754 |
| 19:9853818-9854802 | 19:10113448-10116366 | 19:11105137-11106081 |
| 19:11432219-11433137 | 19:11483050-11484274 | 19:12555041-12556512 |
| 19:12803756-12804739 | 19:12996872-12997823 | 19:13009230-13015488 |
| 19:13016071-13017606 | 19:13762108-13763486 | 19:13932553-13933808 |
| 19:13953568-13954844 | 19:13961041-13963586 | 19:14440991-14441920 |
| 19:14468916-14469908 | 19:14564914-14565816 | 19:15170255-15171231 |
| 19:15173499-15174401 | 19:15279756-15281652 | 19:15364405-15365359 |
| 19:15468747-15470452 | 19:15900705-15901608 | 19:16015008-16015959 |
| 19:16087531-16088958 | 19:16630488-16631390 | 19:16878031-16879002 |
| 19:17194764-17195742 | 19:17322418-17323994 | 19:17351977-17352882 |
| 19:17579935-17580894 | 19:17811288-17812283 | 19:18168456-18170387 |
| 19:18457716-18458618 | 19:18667450-18669323 | 19:18687188-18688095 |
| 19:18770921-18771888 | 19:18774504-18775467 | 19:18780966-18781904 |
| 19:19112205-19113197 | 19:19258762-19259664 | 19:19305602-19306579 |
| 19:19543415-19545489 | 19:21140184-21141859 | 19:21395136-21396038 |
| 19:29612017-29612919 | 19:29663690-29665843 | 19:29678046-29678953 |
| 19:31279033-31279993 | 19:32605125-32606220 | 19:33379210-33380125 |
| 19:34773541-34774514 | 19:35125799-35126782 | 19:35228188-35230287 |
| 19:35527655-35528620 | 19:35557084-35558018 | 19:36113138-36115034 |
| 19:38327009-38327911 | 19:38931869-38933457 | 19:39172476-39173465 |
| 19:39313899-39314873 | 19:39871605-39872565 | 19:39927346-39928317 |
| 19:40192112-40193064 | 19:40321610-40322530 | 19:41576860-41577823 |
| 19:41903761-41904757 | 19:42040996-42041898 | 19:42224990-42225989 |
| 19:42273295-42274247 | 19:42375035-42377399 | 19:42594808-42596144 |
| 19:43413768-43415934 | 19:43718281-43720341 | 19:44808285-44809225 |
| 19:45071215-45072210 | 19:45140031-45140937 | 19:45151516-45152440 |
| 19:45732885-45733868 | 19:45776846-45778279 | 19:45782685-45783940 |
| 19:45884991-45885973 | 19:46693542-46694494 | 19:46755782-46757834 |
| 19:46830963-46832034 | 19:47045184-47046165 | 19:47698788-47699786 |
| 19:48296346-48298023 | 19:48329260-48330251 | 19:48450039-48451013 |
| 19:48454188-48455093 | 19:48661123-48662107 | 19:48672499-48674227 |
| 19:48679688-48681895 | 19:49054662-49055592 | 19:49596271-49597243 |
| 19:49669673-49670586 | 19:49788078-49789050 | 19:49887572-49889154 |
| 19:50210265-50211251 | 19:50477549-50478466 | 19:50517395-50518384 |
| 19:51026959-51028679 | 19:51103403-51104364 | 19:52729379-52730443 |
| 19:53647116-53648825 | 19:53874067-53875002 | 19:53902532-53903444 |
| 19:54313307-54314268 | 19:54459956-54462034 | 19:55345340-55346762 |
| 19:55361639-55364779 | 19:55482592-55484043 | 19:55529424-55531760 |
| 19:55591952-55592914 | 19:56189268-56190400 | 19:56473479-56474381 |
| 19:56537941-56538898 | 19:57253466-57254368 | 19:58366038-58369124 |
| 19:58417292-58418223 | 2:139907-140875 | 2:195755-196700 |
| 2:601376-602840 | 2:619508-622443 | 2:719981-721508 |
| 2:748023-749057 | 2:915104-916717 | 2:1676435-1679190 |
| 2:1727854-1729208 | 2:1815849-1819174 | 2:1842134-1843876 |
| 2:1848948-1849853 | 2:2688556-2689665 | 2:2731602-2732914 |
| 2:2750520-2751491 | 2:2960530-2962522 | 2:3100697-3101673 |
| 2:3176481-3177468 | 2:3188298-3189280 | 2:3195598-3197549 |
| 2:3197474-3198412 | 2:3202868-3203776 | 2:3255718-3257726 |
| 2:3276734-3278309 | 2:3288793-3290743 | 2:3313247-3314444 |
| 2:3387181-3388137 | 2:3389046-3390039 | 2:3421150-3422320 |
| 2:3424439-3425341 | 2:3442765-3444351 | 2:3456009-3456946 |
| 2:3479288-3480226 | 2:3482858-3486685 | 2:3489216-3490407 |
| 2:3500061-3501822 | 2:4135414-4137045 | 2:7846433-7847335 |
| 2:8359406-8360320 | 2:8583274-8585090 | 2:8709387-8710341 |
| 2:9374069-9375389 | 2:9630421-9632390 | 2:10003406-10004689 |
| 2:10057936-10059832 | 2:10388652-10389554 | 2:10425877-10428033 |
| 2:10446296-10448027 | 2:10524106-10527110 | 2:10551088-10552272 |
| 2:10777177-10778232 | 2:10779448-10780413 | 2:11586326-11587589 |
| 2:11633509-11636152 | 2:11656298-11657245 | 2:11669007-11671054 |
| 2:11827671-11828740 | 2:15084445-15085400 | 2:15690249-15691203 |
| 2:20198114-20199034 | 2:20506216-20507145 | 2:23422778-23424106 |
| 2:23527908-23528876 | 2:23560751-23562964 | 2:23630990-23631905 |
| 2:24174370-24176496 | 2:24917744-24921246 | 2:25090480-25091433 |
| 2:25160023-25160937 | 2:25233801-25234840 | 2:25246736-25248583 |
| 2:25328156-25330039 | 2:25595597-25596499 | 2:25980823-25983886 |
| 2:26318844-26319746 | 2:26793370-26794272 | 2:27104248-27105202 |
| 2:27118731-27120706 | 2:27216349-27217858 | 2:27441081-27443381 |
| 2:28456654-28457584 | 2:28649659-28650561 | 2:28925194-28926455 |
| 2:29215686-29216647 | 2:30808385-30809340 | 2:33426516-33427418 |
| 2:36468512-36469414 | 2:36594574-36595531 | 2:36807849-36809208 |
| 2:37187976-37189055 | 2:37195238-37196199 | 2:37282806-37283708 |
| 2:37366883-37367816 | 2:37777154-37778056 | 2:37949180-37950815 |
| 2:44201283-44202221 | 2:44930851-44933493 | 2:44933505-44934409 |
| 2:44954542-44955511 | 2:45309405-45310307 | 2:46428482-46429452 |
| 2:46815787-46817056 | 2:47041467-47042369 | 2:48581840-48582742 |
| 2:48915648-48916631 | 2:52629081-52630055 | 2:53859655-53860603 |
| 2:54254237-54256812 | 2:54663892-54665003 | 2:54727986-54728888 |
| 2:55224717-55225665 | 2:55988734-55990137 | 2:56192414-56193575 |
| 2:58251574-58252526 | 2:60972421-60973391 | 2:61178835-61179737 |
| 2:61184458-61185677 | 2:62192299-62194311 | 2:62235157-62236060 |
| 2:63043861-63045715 | 2:63613594-63614496 | 2:64627434-64628335 |
| 2:65227007-65228006 | 2:65293661-65294608 | 2:65312795-65314708 |
| 2:66531760-66532662 | 2:67214924-67216427 | 2:69519819-69521859 |
| 2:69637262-69638169 | 2:69960904-69964117 | 2:70789877-70791765 |
| 2:73107642-73108589 | 2:73892599-73893563 | 2:74525703-74527566 |
| 2:75666493-75667395 | 2:76697726-76698983 | 2:85258110-85259054 |
| 2:85867112-85869448 | 2:87078314-87079215 | 2:88057923-88058825 |
| 2:88169277-88172405 | 2:88626211-88627171 | 2:95277692-95279041 |
| 2:95349618-95350812 | 2:96238854-96241019 | 2:96835342-96836244 |
| 2:96842254-96845168 | 2:97732540-97736023 | 2:97737049-97738374 |
| 2:98235980-98236926 | 2:98369400-98370959 | 2:98375624-98376582 |
| 2:98387287-98388197 | 2:98821228-98822132 | 2:99254321-99255726 |
| 2:99301610-99302755 | 2:100012344-100013246 | 2:101049912-101051076 |
| 2:101869371-101870335 | 2:102249870-102251775 | 2:102416766-102417887 |
| 2:102616419-102617812 | 2:105241729-105243800 | 2:105868525-105869584 |
| 2:106077422-106078393 | 2:106097391-106098386 | 2:106272895-106273836 |
| 2:106843254-106844156 | 2:108941594-108943729 | 2:109130300-109131243 |
| 2:109196174-109197087 | 2:109334728-109335656 | 2:109376284-109377186 |
| 2:109500718-109503192 | 2:111125387-111126289 | 2:112159122-112160105 |
| 2:118844354-118846364 | 2:119766611-119767513 | 2:120016537-120017517 |
| 2:120297560-120298462 | 2:120791600-120792741 | 2:120796280-120797890 |
| 2:120851822-120852734 | 2:120912516-120913430 | 2:120977554-120979092 |
| 2:120990090-120992244 | 2:121233690-121235341 | 2:121337991-121339121 |
| 2:127042496-127044235 | 2:127592219-127593168 | 2:127713144-127714463 |
| 2:128207511-128208506 | 2:128267694-128268669 | 2:128485339-128487067 |
| 2:130913169-130917651 | 2:131045595-131046513 | 2:131445135-131446126 |
| 2:131487772-131488702 | 2:131723701-131724637 | 2:132669698-132671804 |
| 2:134468674-134470132 | 2:136393832-136394772 | 2:144294721-144295623 |
| 2:144875704-144876667 | 2:158583597-158584578 | 2:161417754-161418705 |
| 2:166311229-166312793 | 2:166495106-166496008 | 2:168006323-168007225 |
| 2:168785789-168786772 | 2:169063041-169063943 | 2:171518775-171519677 |
| 2:172734472-172735375 | 2:173485856-173487104 | 2:174571212-174572326 |
| 2:177601692-177603381 | 2:178531250-178533294 | 2:178575569-178576538 |
| 2:178871967-178872930 | 2:182068107-182069012 | 2:190436510-190437706 |
| 2:191150135-191152206 | 2:191249935-191250837 | 2:192008304-192009226 |
| 2:195655485-195656729 | 2:200768754-200771390 | 2:203237244-203238245 |
| 2:205611441-205612421 | 2:207823978-207825709 | 2:208163223-208164126 |
| 2:208358996-208360948 | 2:212831523-212832870 | 2:215546058-215547026 |
| 2:216002692-216003601 | 2:216674048-216676369 | 2:218402182-218403164 |
| 2:219014830-219015756 | 2:219296824-219297785 | 2:219433307-219437544 |
| 2:219444211-219445621 | 2:219489886-219491071 | 2:219501585-219502543 |
| 2:219626421-219628909 | 2:221616222-221617127 | 2:222311651-222312586 |
| 2:223051729-223053383 | 2:226795948-226797906 | 2:227870493-227871431 |
| 2:230812604-230813515 | 2:230817957-230818921 | 2:230994139-230996727 |
| 2:231256078-231256980 | 2:231593656-231594610 | 2:232420347-232422688 |
| 2:232444841-232445835 | 2:232482204-232483168 | 2:232524797-232527086 |
| 2:232868854-232869823 | 2:233030875-233033302 | 2:233351798-233352784 |
| 2:233461821-233462735 | 2:233472847-233473806 | 2:233475187-233478539 |
| 2:233481358-233482326 | 2:233865730-233866632 | 2:235042269-235043219 |
| 2:235747675-235748747 | 2:236123463-236125256 | 2:236581709-236582611 |
| 2:237264457-237265386 | 2:237387226-237388195 | 2:237536902-237538988 |
| 2:237562855-237564268 | 2:237708184-237709329 | 2:237769137-237770273 |
| 2:237881660-237882607 | 2:238105619-238106596 | 2:238130437-238131420 |
| 2:238140239-238141199 | 2:238167567-238168545 | 2:238438987-238439955 |
| 2:239066737-239067941 | 2:239097241-239099028 | 2:239121670-239123201 |
| 2:239138640-239139921 | 2:239162226-239165020 | 2:239168175-239169104 |
| 2:239189326-239190310 | 2:239192313-239194459 | 2:239199695-239200694 |
| 2:239203837-239204788 | 2:239248663-239249601 | 2:239376276-239377248 |
| 2:239390620-239391602 | 2:239482541-239484778 | 2:239736444-239737393 |
| 2:239955858-239957260 | 2:239987249-239988151 | 2:240042402-240044395 |
| 2:240125797-240126876 | 2:240129276-240130233 | 2:240453383-240457321 |
| 2:240630179-240631423 | 2:240777172-240778730 | 2:240877525-240878479 |
| 2:240895182-240897586 | 2:240968752-240969713 | 2:241048073-241049159 |
| 2:241064572-241066197 | 2:241230070-241231018 | 2:241353223-241354125 |
| 2:241494918-241495878 | 2:241579207-241580109 | 2:241582710-241583682 |
| 2:241645278-241646250 | 2:241665496-241667485 | 2:241669839-241671611 |
| 2:241672201-241673144 | 2:241675436-241679067 | 2:241725203-241727173 |
| 2:241751486-241752691 | 2:241752974-241753876 | 2:241769706-241770660 |
| 2:241795180-241796640 | 2:241892488-241893446 | 2:241900335-241901260 |
| 2:241901136-241902107 | 2:241904687-241905663 | 2:241926326-241927279 |
| 2:241945085-241947267 | 2:241985836-241987673 | 2:241995885-241997483 |
| 2:242054823-242056782 | 20:647983-648893 | 20:761570-762472 |
| 20:1164912-1165814 | 20:1305385-1306362 | 20:1312835-1314705 |
| 20:2839554-2841715 | 20:2863484-2865122 | 20:3225743-3226645 |
| 20:3230261-3232054 | 20:4181964-4182928 | 20:5009153-5011211 |
| 20:6040465-6041367 | 20:6765982-6767155 | 20:6769439-6770410 |
| 20:10668266-10669770 | 20:17990219-17991121 | 20:18187151-18188053 |
| 20:20018081-20019003 | 20:23371893-23372795 | 20:23437962-23440193 |
| 20:25249854-25251568 | 20:31644418-31645320 | 20:31996119-31997257 |
| 20:32458506-32459466 | 20:33790689-33791863 | 20:34988773-34990812 |
| 20:35967583-35968580 | 20:36496956-36497867 | 20:36815040-36816634 |
| 20:37522068-37523785 | 20:37523904-37525528 | 20:38925199-38927835 |
| 20:43944820-43946509 | 20:45889909-45890894 | 20:46250535-46252692 |
| 20:47238569-47239561 | 20:48385915-48386817 | 20:49034040-49035015 |
| 20:49905179-49906690 | 20:50876529-50877431 | 20:51801873-51803302 |
| 20:52151712-52153326 | 20:53658165-53659075 | 20:53944228-53945130 |
| 20:56357914-56358845 | 20:56366496-56367454 | 20:56369284-56370186 |
| 20:57648182-57649097 | 20:57675567-57676539 | 20:58146645-58147583 |
| 20:58710340-58711338 | 20:59039853-59043417 | 20:59931569-59934782 |
| 20:61023030-61024699 | 20:61936180-61937580 | 20:61971201-61972173 |
| 20:62059563-62060530 | 20:62162374-62163345 | 20:62168892-62170606 |
| 20:62172228-62173179 | 20:62192431-62193360 | 20:62304030-62304980 |
| 20:62313918-62314895 | 20:62654449-62655391 | 20:62658166-62659341 |
| 20:62682807-62685019 | 20:62799414-62800889 | 20:62879971-62880903 |
| 20:62882127-62883056 | 20:63238792-63240265 | 20:63318887-63320680 |
| 20:63342267-63343260 | 20:63399849-63400816 | 20:63424319-63425287 |
| 20:63577529-63579025 | 20:63681872-63682788 | 20:63686555-63687779 |
| 20:63860238-63862017 | 20:63862436-63863393 | 20:64073525-64074479 |
| 20:64097981-64098967 | 20:64105647-64108269 | 21:18244076-18246306 |
| 21:25361313-25362385 | 21:25606096-25607069 | 21:26836245-26837147 |
| 21:30281236-30282138 | 21:31251326-31252848 | 21:31657990-31658891 |
| 21:32282208-32283110 | 21:33169680-33171828 | 21:33488150-33489052 |
| 21:33912831-33913798 | 21:34722033-34723609 | 21:35886727-35887667 |
| 21:36244928-36245907 | 21:36245978-36246964 | 21:36989913-36990893 |
| 21:37563305-37565208 | 21:37667024-37668512 | 21:38270980-38272883 |
| 21:38751902-38753442 | 21:39866043-39868013 | 21:41320352-41321254 |
| 21:41687021-41687988 | 21:41688669-41689935 | 21:41756107-41757080 |
| 21:41799778-41802225 | 21:41804642-41805596 | 21:41811580-41813004 |
| 21:42127311-42128246 | 21:42302140-42303042 | 21:42305504-42307407 |
| 21:42685819-42686809 | 21:42774778-42775714 | 21:43657923-43658983 |
| 21:43676103-43677026 | 21:43772788-43774600 | 21:43811839-43814232 |
| 21:43943366-43944340 | 21:43979009-43979965 | 21:44292504-44293466 |
| 21:44310334-44311250 | 21:44321019-44321972 | 21:44462246-44463157 |
| 21:44499226-44500157 | 21:44571741-44572681 | 21:44625615-44626955 |
| 21:44695346-44696248 | 21:44947486-44948946 | 21:44954090-44955024 |
| 21:44956376-44957287 | 21:44957913-44958871 | 21:44994103-44995082 |
| 21:45017067-45020102 | 21:45269551-45270545 | 21:45296369-45297271 |
| 21:45305831-45306733 | 21:45366738-45368437 | 21:45424508-45427018 |
| 21:45432948-45433862 | 21:45439527-45441257 | 21:45447483-45448672 |
| 21:45472375-45473335 | 21:45474724-45476806 | 21:45492042-45493164 |
| 21:45497076-45498974 | 21:45866597-45867550 | 21:46032833-46034476 |
| 21:46035020-46039209 | 21:46112764-46113716 | 21:46124780-46125725 |
| 21:46180693-46181681 | 21:46386585-46387505 | 21:46410512-46412170 |
| 21:46434626-46436824 | 21:46549423-46550364 | 22:17365977-17368227 |
| 22:17748150-17749609 | 22:18152176-18153131 | 22:19565988-19567286 |
| 22:19979489-19980479 | 22:20143554-20144456 | 22:20584302-20585204 |
| 22:21930482-21931384 | 22:22025159-22026957 | 22:22556236-22557153 |
| 22:22558325-22560299 | 22:23095700-23096681 | 22:23401379-23403486 |
| 22:23688880-23689782 | 22:23835474-23837461 | 22:24187670-24188948 |
| 22:24493819-24496205 | 22:25359533-25361140 | 22:26498945-26499929 |
| 22:27796270-27797199 | 22:29049377-29050303 | 22:29259452-29260354 |
| 22:29315388-29317013 | 22:30243348-30244296 | 22:30378493-30379395 |
| 22:30555420-30556766 | 22:30967306-30969704 | 22:31099797-31100707 |
| 22:31289614-31291247 | 22:31906042-31907276 | 22:32254460-32256026 |
| 22:33651103-33652005 | 22:35423624-35424813 | 22:35551169-35552668 |
| 22:37642409-37643389 | 22:37821701-37823908 | 22:37847397-37848299 |
| 22:38079830-38084601 | 22:38110065-38111225 | 22:38119687-38121930 |
| 22:38132053-38133475 | 22:38494457-38495428 | 22:39381210-39382128 |
| 22:39656679-39659113 | 22:39893011-39895423 | 22:40421088-40421990 |
| 22:40677617-40679476 | 22:41245343-41246245 | 22:41365772-41366674 |
| 22:41394956-41396346 | 22:41542742-41543645 | 22:41697525-41700465 |
| 22:41939746-41942157 | 22:42077253-42078210 | 22:42274857-42275793 |
| 22:42372583-42373499 | 22:42431569-42434279 | 22:42513621-42514616 |
| 22:42692194-42694441 | 22:43998270-44000004 | 22:44686782-44687685 |
| 22:44736355-44738303 | 22:44914247-44915149 | 22:45007080-45008069 |
| 22:45184558-45185546 | 22:45200526-45201550 | 22:45202465-45203774 |
| 22:45501352-45504380 | 22:45562821-45563735 | 22:46112041-46114280 |
| 22:46213956-46215493 | 22:46261409-46262311 | 22:46300086-46301003 |
| 22:46409226-46410996 | 22:46536214-46537198 | 22:46793793-46794695 |
| 22:47060570-47061666 | 22:47117080-47118022 | 22:48743171-48745859 |
| 22:48749804-48751743 | 22:49540258-49541982 | 22:49615519-49617047 |
| 22:49631971-49632966 | 22:49775080-49776743 | 22:49783600-49785053 |
| 22:49824175-49825491 | 22:49926812-49927764 | 22:50024659-50025561 |
| 22:50072858-50073788 | 22:50076495-50077491 | 22:50090094-50092501 |
| 22:50183481-50185286 | 22:50208008-50209197 | 22:50220457-50221421 |
| 22:50281488-50283745 | 22:50288034-50288997 | 22:50323977-50325659 |
| 22:50406279-50407458 | 22:50408618-50409552 | 22:50434375-50435616 |
| 22:50453975-50454958 | 22:50549480-50550430 | 22:50561068-50562059 |
| 22:50577523-50580481 | 22:50703924-50705374 | 22:50739012-50739989 |
| 3:4672705-4673836 | 3:6862156-6863144 | 3:8606418-8607339 |
| 3:8985341-8986335 | 3:9685140-9686042 | 3:9746791-9747877 |
| 3:9927807-9928763 | 3:10107738-10108991 | 3:10285317-10286284 |
| 3:10289946-10292015 | 3:11298258-11299160 | 3:11474979-11476493 |
| 3:11565971-11569688 | 3:11609521-11610685 | 3:11680261-11681430 |
| 3:11721363-11722684 | 3:12539629-12540700 | 3:12581837-12582768 |
| 3:12816249-12817867 | 3:12898271-12900474 | 3:12905644-12906774 |
| 3:12965236-12968543 | 3:13041101-13042423 | 3:13071910-13074859 |
| 3:13389582-13390691 | 3:13935868-13936774 | 3:14303794-14304730 |
| 3:14484452-14485483 | 3:15498112-15500303 | 3:16147836-16148785 |
| 3:16244326-16245238 | 3:16425679-16426668 | 3:16882529-16884765 |
| 3:22989323-22991308 | 3:24122138-24123040 | 3:25173941-25174866 |
| 3:29701160-29702139 | 3:30720278-30721180 | 3:31452181-31453498 |
| 3:32592301-32593429 | 3:33090567-33091469 | 3:33439870-33440865 |
| 3:37397764-37399375 | 3:37834377-37835356 | 3:38304583-38307075 |
| 3:39279419-39283286 | 3:40292529-40293431 | 3:42089669-42091368 |
| 3:42202070-42203514 | 3:42222606-42223565 | 3:42657185-42660739 |
| 3:42691584-42692793 | 3:42773925-42774862 | 3:42904593-42909140 |
| 3:43079647-43080612 | 3:43485198-43486115 | 3:43605610-43606577 |
| 3:43617664-43618756 | 3:44241000-44242866 | 3:44711109-44712011 |
| 3:44734346-44735248 | 3:45010941-45011872 | 3:45086851-45087824 |
| 3:45678184-45679154 | 3:45942357-45944127 | 3:46263411-46264350 |
| 3:46265530-46266977 | 3:46409000-46409902 | 3:46467478-46468380 |
| 3:46563983-46565260 | 3:46629370-46631397 | 3:46676389-46679180 |
| 3:46688929-46689860 | 3:46749449-46751518 | 3:47353017-47353919 |
| 3:47845637-47847194 | 3:47847997-47848921 | 3:48195569-48196525 |
| 3:48268591-48269560 | 3:48370949-48373356 | 3:48414761-48415825 |
| 3:48475122-48476049 | 3:48639685-48640668 | 3:48642410-48643312 |
| 3:49530703-49533297 | 3:50191632-50192592 | 3:51393989-51394916 |
| 3:51715225-51716309 | 3:52064497-52066085 | 3:52221163-52223498 |
| 3:52491696-52492670 | 3:52515702-52516639 | 3:52518492-52519466 |
| 3:52521735-52522700 | 3:53809594-53810541 | 3:53810727-53812428 |
| 3:53881841-53883967 | 3:54864150-54865051 | 3:54927378-54929610 |
| 3:56683240-56684221 | 3:57553034-57553936 | 3:58122827-58123905 |
| 3:58176691-58178983 | 3:58501092-58502077 | 3:59404979-59406060 |
| 3:63894643-63896487 | 3:64098725-64099678 | 3:64217049-64217959 |
| 3:69958817-69959911 | 3:69998978-70000310 | 3:70984794-70985696 |
| 3:71370112-71371023 | 3:71494026-71494981 | 3:71573052-71573960 |
| 3:72237124-72238053 | 3:72720096-72721020 | 3:72956582-72957484 |
| 3:100159776-100160678 | 3:100550912-100551895 | 3:101964270-101965176 |
| 3:102190058-102191000 | 3:107881735-107882887 | 3:109409596-109410519 |
| 3:111540895-111542614 | 3:111731416-111732318 | 3:112185420-112186496 |
| 3:113943917-113944870 | 3:114290372-114291337 | 3:114351032-114352025 |
| 3:114411464-114412418 | 3:119145289-119147174 | 3:119595963-119597231 |
| 3:119659922-119661134 | 3:119912815-119913717 | 3:120165802-120166734 |
| 3:120396415-120397585 | 3:120780608-120781568 | 3:122680038-122682947 |
| 3:122739498-122740403 | 3:122885427-122886959 | 3:122915067-122916410 |
| 3:124031560-124032462 | 3:125211432-125212427 | 3:125958607-125960626 |
| 3:126056025-126058045 | 3:126355516-126356493 | 3:126489017-126490259 |
| 3:126608143-126610288 | 3:126746653-126747555 | 3:127192365-127193550 |
| 3:127604449-127605417 | 3:127674166-127675121 | 3:127675524-127677073 |
| 3:128279418-128281435 | 3:128360267-128363119 | 3:128424270-128427686 |
| 3:129526890-129529256 | 3:129556277-129557876 | 3:129570451-129572293 |
| 3:130025482-130026420 | 3:130030102-130031054 | 3:130348693-130349609 |
| 3:131385530-131386432 | 3:132536756-132537658 | 3:133930444-133931515 |
| 3:136150704-136151935 | 3:138759908-138760810 | 3:138936657-138937640 |
| 3:138959292-138961278 | 3:140352093-140352995 | 3:140681607-140683839 |
| 3:141064836-141066868 | 3:141455007-141455933 | 3:143120854-143123328 |
| 3:143807462-143808364 | 3:149248623-149249544 | 3:149378550-149379492 |
| 3:149463872-149465713 | 3:149982576-149983505 | 3:150050313-150051293 |
| 3:150390576-150391498 | 3:150518900-150521221 | 3:150778346-150780346 |
| 3:151280303-151281205 | 3:151458122-151459347 | 3:155182644-155183546 |
| 3:157541849-157542751 | 3:160001578-160002480 | 3:160676834-160678083 |
| 3:164003711-164004670 | 3:169309271-169310227 | 3:169381190-169382750 |
| 3:170062374-170064840 | 3:171381468-171382400 | 3:171841672-171842574 |
| 3:172398393-172399295 | 3:177529693-177530654 | 3:179024325-179025297 |
| 3:179403547-179404487 | 3:180827798-180830694 | 3:183269562-183270559 |
| 3:183424116-183425060 | 3:184295844-184297310 | 3:184324378-184325280 |
| 3:184326778-184327680 | 3:184367848-184368750 | 3:184488276-184489244 |
| 3:184575030-184576022 | 3:184576549-184577497 | 3:184580513-184581434 |
| 3:185473187-185474089 | 3:185496496-185497429 | 3:185656299-185657236 |
| 3:186635063-186636405 | 3:186671205-186672107 | 3:186854112-186855014 |
| 3:187235390-187236356 | 3:187701606-187703872 | 3:190120036-190123025 |
| 3:190545846-190546748 | 3:192850420-192851323 | 3:193591697-193593647 |
| 3:193696426-193697328 | 3:194203180-194206239 | 3:194385680-194386737 |
| 3:195061497-195064127 | 3:195753249-195754206 | 3:195810089-195811065 |
| 3:195811298-195814681 | 3:195837327-195838596 | 3:195876284-195877268 |
| 3:196283074-196283981 | 3:196566537-196567455 | 3:196659665-196661129 |
| 3:197001112-197002539 | 3:197393996-197395064 | 3:197417073-197418021 |
| 3:197458789-197459712 | 3:197511502-197512847 | 3:197674831-197675769 |
| 3:197856915-197857816 | 3:197912377-197913593 | 3:197936413-197937549 |
| 4:525266-526934 | 4:623841-626420 | 4:713550-714513 |
| 4:719619-721083 | 4:724708-725639 | 4:727627-729041 |
| 4:742089-744931 | 4:756881-757864 | 4:835439-836389 |
| 4:851338-852324 | 4:887533-888456 | 4:899673-900633 |
| 4:901449-902414 | 4:908767-910307 | 4:910911-913621 |
| 4:914075-915615 | 4:948017-949415 | 4:955232-956195 |
| 4:957735-959125 | 4:964451-965400 | 4:969566-970773 |
| 4:991270-992255 | 4:1014211-1015199 | 4:1021353-1023431 |
| 4:1177739-1178698 | 4:1201723-1202882 | 4:1211819-1212804 |
| 4:1215727-1216716 | 4:1226623-1229144 | 4:1229721-1230903 |
| 4:1237785-1238765 | 4:1251326-1252317 | 4:1278748-1279722 |
| 4:1308123-1309085 | 4:1328356-1329312 | 4:1359070-1362175 |
| 4:1363236-1364232 | 4:1372538-1374605 | 4:1376741-1377703 |
| 4:1379656-1380638 | 4:1381868-1382786 | 4:1554317-1555656 |
| 4:1559983-1560952 | 4:1602213-1603395 | 4:1612997-1615235 |
| 4:1622734-1624530 | 4:1800807-1801783 | 4:1803414-1804966 |
| 4:1805009-1807709 | 4:1832401-1833394 | 4:1842537-1843475 |
| 4:1947734-1948991 | 4:1950493-1951674 | 4:1954829-1956678 |
| 4:1974042-1975114 | 4:1975142-1976115 | 4:1980477-1982174 |
| 4:1984514-1988149 | 4:2250017-2250961 | 4:2273534-2275763 |
| 4:2300680-2303494 | 4:2304150-2305810 | 4:2339018-2339982 |
| 4:2363925-2365950 | 4:2399330-2400793 | 4:2731701-2732690 |
| 4:2745750-2747116 | 4:2791596-2794319 | 4:2824077-2825867 |
| 4:2826750-2827724 | 4:2927377-2929036 | 4:2998709-2999680 |
| 4:3231539-3233231 | 4:3238498-3239460 | 4:3248044-3249568 |
| 4:3252780-3253768 | 4:3255199-3256841 | 4:3307169-3308995 |
| 4:3312770-3314253 | 4:3339842-3341540 | 4:3372625-3374226 |
| 4:3411406-3412508 | 4:3422999-3425347 | 4:3444135-3445120 |
| 4:3447056-3448718 | 4:3511585-3513589 | 4:3614607-3616379 |
| 4:3641137-3643507 | 4:4036480-4037432 | 4:4427037-4427939 |
| 4:5053648-5055355 | 4:5218097-5218999 | 4:5456298-5457633 |
| 4:6326113-6327045 | 4:6420759-6422171 | 4:6594111-6595114 |
| 4:6955593-6956515 | 4:7023584-7024486 | 4:7024745-7025719 |
| 4:7046663-7047620 | 4:7054068-7055578 | 4:7162580-7163520 |
| 4:7785691-7786660 | 4:7787267-7788914 | 4:7983639-7984629 |
| 4:8125102-8126097 | 4:8250963-8252523 | 4:8289661-8291137 |
| 4:8374632-8375623 | 4:8383947-8385068 | 4:8386169-8387151 |
| 4:8387581-8388483 | 4:8415736-8416948 | 4:8465930-8466879 |
| 4:8475695-8476617 | 4:8605678-8607309 | 4:8617727-8618978 |
| 4:9492301-9493247 | 4:10082550-10083552 | 4:10118605-10119509 |
| 4:16075193-16076681 | 4:16256061-16257043 | 4:16360199-16361593 |
| 4:16607396-16608317 | 4:17781764-17782739 | 4:20883172-20884370 |
| 4:22387860-22389034 | 4:25862144-25864606 | 4:37958723-37961035 |
| 4:38119784-38120723 | 4:40195761-40197702 | 4:40333722-40336352 |
| 4:40425558-40426534 | 4:40437279-40438968 | 4:40749241-40751770 |
| 4:40815417-40816319 | 4:41503474-41504376 | 4:41981143-41983499 |
| 4:47706122-47707065 | 4:69781785-69782974 | 4:70522347-70523249 |
| 4:75561699-75562601 | 4:75629815-75631697 | 4:76010450-76011745 |
| 4:76073782-76074792 | 4:76740115-76742381 | 4:77112235-77114634 |
| 4:77172284-77173186 | 4:79325080-79326639 | 4:79964282-79965536 |
| 4:92182312-92183307 | 4:95839343-95840989 | 4:99161362-99162625 |
| 4:99649935-99654013 | 4:102577491-102578401 | 4:106256217-106257125 |
| 4:109462732-109463655 | 4:112411352-112412537 | 4:118723378-118724280 |
| 4:121932513-121933701 | 4:122378518-122380696 | 4:122924603-122925505 |
| 4:123547161-123548123 | 4:139859505-139860827 | 4:141219492-141221818 |
| 4:142034855-142035757 | 4:145761061-145762020 | 4:147479748-147482296 |
| 4:150175535-150176469 | 4:150581332-150584823 | 4:151662852-151663790 |
| 4:151700846-151701770 | 4:151718406-151719308 | 4:152973135-152974483 |
| 4:152975899-152976875 | 4:153155160-153156930 | 4:153295218-153296192 |
| 4:159034661-159035585 | 4:163473139-163474115 | 4:165006848-165008187 |
| 4:165031414-165032680 | 4:169754960-169755862 | 4:169991005-169992766 |
| 4:173527848-173528751 | 4:177322128-177323048 | 4:182565832-182566734 |
| 4:182764584-182765486 | 4:182856503-182857870 | 4:183263363-183265999 |
| 4:183315635-183316723 | 4:183321306-183322272 | 4:183512347-183513283 |
| 4:183985537-183986439 | 4:184000762-184001963 | 4:184008546-184011629 |
| 4:184390192-184391104 | 4:184630616-184631543 | 4:184803167-184804097 |
| 4:184950825-184952373 | 4:185149590-185150543 | 4:185622966-185624700 |
| 4:186157162-186158122 | 4:186619210-186620926 | 4:186699527-186701560 |
| 4:189103122-189104046 | 5:192013-192946 | 5:204571-206352 |
| 5:307233-308204 | 5:389118-390078 | 5:398351-400593 |
| 5:455927-456880 | 5:465894-466856 | 5:485829-486796 |
| 5:503983-504947 | 5:536694-539363 | 5:600176-602580 |
| 5:619089-620843 | 5:625042-626009 | 5:640017-640919 |
| 5:661634-662541 | 5:862675-863602 | 5:871601-872566 |
| 5:876304-877206 | 5:886598-887537 | 5:888901-890050 |
| 5:1075939-1076905 | 5:1083313-1084292 | 5:1088345-1090197 |
| 5:1092415-1093389 | 5:1099009-1100808 | 5:1120023-1120991 |
| 5:1216642-1220393 | 5:1259884-1260796 | 5:1277382-1280730 |
| 5:1284852-1286789 | 5:1323496-1326461 | 5:1335280-1336247 |
| 5:1381519-1382969 | 5:1465767-1466762 | 5:1476609-1477558 |
| 5:1537539-1538511 | 5:1744882-1747563 | 5:1768749-1770459 |
| 5:1808930-1809832 | 5:1863868-1867078 | 5:2155200-2156609 |
| 5:3535206-3536877 | 5:3740331-3741329 | 5:4144234-4145150 |
| 5:4580841-4581836 | 5:4865397-4866878 | 5:6604496-6605454 |
| 5:6750198-6751167 | 5:10290600-10291551 | 5:10874946-10875927 |
| 5:11068413-11069362 | 5:14379869-14380838 | 5:14404983-14407084 |
| 5:14409613-14410754 | 5:14460494-14461473 | 5:14487647-14488820 |
| 5:14497829-14498731 | 5:14506312-14509374 | 5:16776719-16777646 |
| 5:16783371-16785576 | 5:17275751-17276655 | 5:17353844-17354962 |
| 5:19987380-19988704 | 5:31944349-31945265 | 5:32016618-32017546 |
| 5:32713360-32714288 | 5:33984004-33985114 | 5:35859747-35860649 |
| 5:36240555-36241522 | 5:37248765-37251359 | 5:37771667-37772569 |
| 5:38134339-38135619 | 5:38444667-38446633 | 5:41730048-41730950 |
| 5:42468126-42469271 | 5:43556041-43559956 | 5:43612516-43613485 |
| 5:43648726-43649628 | 5:54454537-54456510 | 5:56111264-56112219 |
| 5:57421985-57423864 | 5:57434895-57435797 | 5:60487351-60488253 |
| 5:67163218-67164215 | 5:68899192-68900094 | 5:69419105-69420399 |
| 5:73119096-73121897 | 5:73435682-73436678 | 5:73812943-73814074 |
| 5:73909186-73910169 | 5:74081042-74081944 | 5:75056734-75057977 |
| 5:75831523-75832640 | 5:78815515-78816417 | 5:78838272-78839174 |
| 5:79689077-79690885 | 5:79838719-79839698 | 5:80299953-80300906 |
| 5:80351291-80352893 | 5:81225273-81226207 | 5:89722106-89723032 |
| 5:94569102-94570089 | 5:96432194-96433919 | 5:102498160-102499819 |
| 5:107359853-107360755 | 5:109883425-109885996 | 5:111090912-111091814 |
| 5:113432942-113436121 | 5:114690628-114691530 | 5:115178485-115179625 |
| 5:116446227-116447754 | 5:124643076-124644037 | 5:126446530-126447432 |
| 5:126827390-126828292 | 5:127228366-127230457 | 5:127651682-127653697 |
| 5:132071977-132074491 | 5:132776209-132779456 | 5:132813175-132815806 |
| 5:133342612-133343543 | 5:134899474-134900376 | 5:135189890-135192309 |
| 5:135270092-135270994 | 5:136022708-136023610 | 5:136027920-136030662 |
| 5:137806992-137808562 | 5:138467121-138468094 | 5:139394646-139396584 |
| 5:142828320-142829222 | 5:143206385-143207564 | 5:147226569-147227559 |
| 5:147901208-147902189 | 5:149129453-149130355 | 5:149257942-149258844 |
| 5:149313797-149314752 | 5:150297135-150298522 | 5:150619947-150621052 |
| 5:150646446-150648267 | 5:151303027-151305067 | 5:151803627-151804534 |
| 5:151924214-151925801 | 5:154058009-154058968 | 5:154474292-154475852 |
| 5:154689659-154692615 | 5:157986125-157987470 | 5:164600746-164601851 |
| 5:165231166-165232138 | 5:168246428-168248272 | 5:168260646-168263183 |
| 5:170232163-170234280 | 5:171308835-171310017 | 5:171456120-171457871 |
| 5:171458605-171459510 | 5:171482514-171483416 | 5:172095109-172096737 |
| 5:172639414-172640532 | 5:175682827-175685056 | 5:175697810-175698721 |
| 5:175868794-175870729 | 5:176527061-176529472 | 5:176880192-176881314 |
| 5:177090510-177091453 | 5:177091856-177094051 | 5:177094997-177096894 |
| 5:177435979-177436968 | 5:178462445-178464671 | 5:178618807-178619709 |
| 5:178792690-178793672 | 5:179153017-179154654 | 5:179222982-179225026 |
| 5:179475208-179476139 | 5:179875373-179876346 | 5:179878084-179878986 |
| 5:179918377-179919332 | 5:180136718-180139047 | 5:180327836-180328820 |
| 5:180605966-180607077 | 5:180849239-180850141 | 5:180897326-180899735 |
| 5:181220196-181221785 | 6:230391-231476 | 6:446961-447936 |
| 6:469979-471241 | 6:595550-596809 | 6:688642-689544 |
| 6:774835-776025 | 6:1514785-1516859 | 6:1526292-1527236 |
| 6:1577304-1578409 | 6:1615059-1616036 | 6:1634803-1636151 |
| 6:1707876-1708827 | 6:1765263-1766877 | 6:3268729-3270848 |
| 6:3329068-3330218 | 6:3340215-3341189 | 6:3444487-3445473 |
| 6:4705467-4706688 | 6:4891879-4892859 | 6:5109996-5111012 |
| 6:5144386-5145362 | 6:6534071-6535028 | 6:6613291-6615076 |
| 6:6842502-6843407 | 6:7228480-7230556 | 6:7584967-7586962 |
| 6:7986974-7987876 | 6:8086072-8087400 | 6:10409512-10410497 |
| 6:11199757-11201242 | 6:11429892-11430794 | 6:11767311-11768213 |
| 6:13193282-13194228 | 6:13364274-13365260 | 6:14639867-14640769 |
| 6:14754614-14755563 | 6:15142467-15143398 | 6:15524032-15524983 |
| 6:16326017-16326943 | 6:17168702-17169604 | 6:17709024-17709926 |
| 6:18260843-18262401 | 6:18693498-18694476 | 6:22146661-22147598 |
| 6:22569771-22571461 | 6:24534525-24535427 | 6:25725758-25727600 |
| 6:26015942-26018860 | 6:26565193-26566188 | 6:28583009-28584393 |
| 6:28588801-28590228 | 6:28902497-28904759 | 6:28976874-28977849 |
| 6:28994542-28996572 | 6:29003028-29005744 | 6:29457383-29458994 |
| 6:29602379-29603348 | 6:29748619-29752243 | 6:29955897-29957206 |
| 6:30161312-30165389 | 6:30188784-30193313 | 6:30197377-30200126 |
| 6:30203914-30208781 | 6:30263411-30265488 | 6:30322030-30324442 |
| 6:30339839-30342995 | 6:30655862-30657529 | 6:30931087-30933862 |
| 6:31132956-31133897 | 6:31161016-31163222 | 6:31170399-31172304 |
| 6:31490618-31491581 | 6:31507066-31510910 | 6:31527732-31528750 |
| 6:31761393-31762640 | 6:31809291-31814914 | 6:31894697-31895883 |
| 6:31962578-31963516 | 6:32050814-32053890 | 6:32086671-32092279 |
| 6:32120335-32121237 | 6:32165656-32167905 | 6:32209363-32215690 |
| 6:32215968-32221939 | 6:32394048-32397536 | 6:32826860-32831561 |
| 6:32975967-32977169 | 6:33170441-33186335 | 6:33191125-33194152 |
| 6:33195338-33196269 | 6:33209486-33214337 | 6:33258660-33259587 |
| 6:33263277-33266909 | 6:33274892-33281769 | 6:33311273-33317021 |
| 6:33318312-33322393 | 6:33413428-33414541 | 6:33687489-33689593 |
| 6:33802608-33805040 | 6:35492923-35493882 | 6:35511708-35513687 |
| 6:35513802-35514704 | 6:35774848-35776472 | 6:35794564-35795509 |
| 6:36335307-36337829 | 6:36968248-36969185 | 6:37137104-37138007 |
| 6:38722272-38723496 | 6:39791840-39792759 | 6:41335001-41336353 |
| 6:41508456-41509358 | 6:41569210-41570165 | 6:41592868-41594997 |
| 6:41806225-41807215 | 6:42025759-42026733 | 6:42175156-42176107 |
| 6:42258825-42259771 | 6:43055871-43056848 | 6:43262487-43263528 |
| 6:43933278-43934223 | 6:44151339-44153102 | 6:44228174-44229076 |
| 6:44313561-44314863 | 6:44621876-44622778 | 6:44651977-44652879 |
| 6:44727860-44728778 | 6:44729214-44730161 | 6:46551936-46552838 |
| 6:46686305-46689758 | 6:49620890-49621792 | 6:49636204-49638034 |
| 6:53752435-53753337 | 6:57045063-57045977 | 6:71583635-71586136 |
| 6:73156576-73157541 | 6:73353808-73355288 | 6:73360792-73362208 |
| 6:73368757-73370988 | 6:84765962-84766940 | 6:88168850-88170589 |
| 6:90233369-90234272 | 6:90586189-90587165 | 6:99607455-99608436 |
| 6:99608319-99609261 | 6:107733163-107734275 | 6:108698537-108699501 |
| 6:110520237-110521139 | 6:110645619-110646560 | 6:111566966-111567906 |
| 6:111625112-111626014 | 6:112346327-112347780 | 6:113644987-113645890 |
| 6:116617326-116618266 | 6:118709728-118711490 | 6:123800979-123802261 |
| 6:125154780-125155682 | 6:128579881-128580860 | 6:129860415-129861686 |
| 6:132904487-132905389 | 6:136360212-136361548 | 6:137042498-137045935 |
| 6:137173766-137174668 | 6:138333759-138335577 | 6:139166068-139167049 |
| 6:143336922-143337845 | 6:146598220-146599329 | 6:149491763-149492724 |
| 6:149910287-149911587 | 6:150733412-150734314 | 6:150936652-150938279 |
| 6:151341733-151342688 | 6:151350280-151351236 | 6:154404557-154407071 |
| 6:154554750-154555664 | 6:154602707-154603609 | 6:154875359-154876318 |
| 6:154993509-154994436 | 6:155256121-155257429 | 6:155263725-155264627 |
| 6:155708584-155709710 | 6:157134178-157135080 | 6:157206741-157208424 |
| 6:157325101-157326155 | 6:157596472-157597687 | 6:157673246-157674294 |
| 6:157839421-157840336 | 6:158042817-158044341 | 6:158310773-158314181 |
| 6:158514616-158516999 | 6:158572940-158573919 | 6:158628197-158629108 |
| 6:158630830-158632393 | 6:158766402-158767895 | 6:158923251-158924193 |
| 6:159035027-159037163 | 6:159129811-159130770 | 6:159130861-159131763 |
| 6:159217966-159218948 | 6:159799221-159801249 | 6:159996116-159997018 |
| 6:160069670-160070584 | 6:160158088-160159023 | 6:161154102-161155063 |
| 6:161216455-161217357 | 6:161234830-161235769 | 6:163149114-163150329 |
| 6:163300018-163301004 | 6:163671023-163672407 | 6:163833582-163835524 |
| 6:163873207-163874163 | 6:164336482-164337797 | 6:166297188-166298550 |
| 6:166445709-166446687 | 6:166454914-166455867 | 6:166486396-166487448 |
| 6:166551485-166552459 | 6:166604007-166604940 | 6:166618464-166619450 |
| 6:166721138-166723464 | 6:166899231-166900223 | 6:166900948-166901907 |
| 6:166901948-166902935 | 6:166952680-166953615 | 6:167130928-167131899 |
| 6:167235223-167236804 | 6:167294846-167296683 | 6:167738038-167738970 |
| 6:167974952-167976499 | 6:167977332-167978980 | 6:167985830-167988493 |
| 6:167988480-167990652 | 6:167990422-167991365 | 6:167998649-168000071 |
| 6:168041785-168043818 | 6:168043959-168046423 | 6:168189911-168191257 |
| 6:168232264-168233688 | 6:168285069-168286040 | 6:168384224-168385694 |
| 6:168411511-168415466 | 6:168443515-168445056 | 6:168573929-168575442 |
| 6:168824242-168826236 | 6:169161814-169162992 | 6:169185865-169186790 |
| 6:169423410-169425107 | 6:169500343-169503115 | 6:169627468-169628461 |
| 6:169646908-169648844 | 6:169660377-169661331 | 6:169666253-169667216 |
| 6:170085353-170087485 | 6:170098403-170099356 | 6:170165633-170168347 |
| 6:170229726-170230695 | 6:170244600-170246730 | 6:170269005-170273974 |
| 6:170280923-170283847 | 6:170464912-170465856 | 7:167966-172087 |
| 7:194498-196222 | 7:205209-206114 | 7:208907-215304 |
| 7:225915-228652 | 7:500021-500983 | 7:504394-505738 |
| 7:511966-514719 | 7:541599-542553 | 7:552476-555460 |
| 7:555882-557100 | 7:568206-572399 | 7:572453-574000 |
| 7:594865-598346 | 7:600197-603032 | 7:604114-606664 |
| 7:631570-632564 | 7:632349-633325 | 7:676642-677609 |
| 7:690513-692426 | 7:738558-740634 | 7:744866-745822 |
| 7:751532-753513 | 7:763266-764170 | 7:784717-785652 |
| 7:822248-823691 | 7:832119-833105 | 7:843328-844330 |
| 7:853895-854840 | 7:890276-891874 | 7:893974-894891 |
| 7:903503-905687 | 7:906569-911379 | 7:919576-922359 |
| 7:958522-961108 | 7:966659-967607 | 7:978205-979107 |
| 7:990013-992304 | 7:998787-1001171 | 7:1004866-1006324 |
| 7:1057199-1058155 | 7:1084941-1087655 | 7:1099892-1103269 |
| 7:1114370-1115321 | 7:1314607-1315862 | 7:1441281-1443429 |
| 7:1450467-1453358 | 7:1470012-1471233 | 7:1478513-1479982 |
| 7:1481201-1482175 | 7:1483400-1484380 | 7:1535434-1537044 |
| 7:1547154-1551462 | 7:1654411-1655787 | 7:1707917-1710521 |
| 7:1742430-1746068 | 7:1746302-1748853 | 7:1758696-1760090 |
| 7:1769173-1770167 | 7:1809506-1812446 | 7:1844270-1845934 |
| 7:1846102-1847861 | 7:1868161-1869509 | 7:1896098-1899704 |
| 7:1899893-1900823 | 7:1906360-1907350 | 7:1910528-1911500 |
| 7:1951683-1952585 | 7:1956887-1958567 | 7:1985035-1988381 |
| 7:1989000-1989985 | 7:2004413-2005315 | 7:2012245-2014184 |
| 7:2016911-2017852 | 7:2039821-2040977 | 7:2044135-2045116 |
| 7:2051958-2052863 | 7:2073225-2074747 | 7:2074704-2076342 |
| 7:2076646-2081187 | 7:2081982-2083489 | 7:2087671-2089604 |
| 7:2095779-2097758 | 7:2098538-2101137 | 7:2108648-2115630 |
| 7:2144639-2148051 | 7:2154614-2156156 | 7:2210102-2211323 |
| 7:2219869-2220786 | 7:2254572-2256046 | 7:2374320-2375266 |
| 7:2526587-2527516 | 7:2538130-2539583 | 7:2542514-2544409 |
| 7:2582417-2584780 | 7:2587261-2588617 | 7:2655034-2655983 |
| 7:2700761-2701663 | 7:2723743-2726197 | 7:2730239-2731589 |
| 7:2819466-2820559 | 7:2905750-2907307 | 7:2993059-2994491 |
| 7:3041851-3042753 | 7:3437919-3438821 | 7:4147676-4148645 |
| 7:4174582-4175908 | 7:4224809-4226704 | 7:4259540-4262126 |
| 7:4706049-4708610 | 7:4754764-4755726 | 7:4757677-4758638 |
| 7:4762360-4763348 | 7:4763683-4765856 | 7:4783699-4785888 |
| 7:4790233-4791198 | 7:5117668-5118646 | 7:5212914-5214991 |
| 7:5226840-5228490 | 7:5332394-5333372 | 7:5481654-5482583 |
| 7:5494368-5496839 | 7:5600940-5602885 | 7:5605811-5606777 |
| 7:6157359-6158585 | 7:6163008-6165425 | 7:6167948-6169126 |
| 7:6171549-6173692 | 7:6525711-6527528 | 7:8113195-8114145 |
| 7:9725813-9726783 | 7:10972876-10974197 | 7:16397289-16398837 |
| 7:16399011-16399980 | 7:16462411-16463393 | 7:22664161-22666507 |
| 7:23105096-23106082 | 7:23585623-23586992 | 7:23678976-23680772 |
| 7:24978346-24980572 | 7:25851271-25852174 | 7:26244237-26245425 |
| 7:26636121-26638895 | 7:27087882-27088868 | 7:27147393-27148388 |
| 7:27154879-27157736 | 7:28279191-28280126 | 7:29478320-29479237 |
| 7:30851699-30852619 | 7:30923845-30924747 | 7:30925323-30926262 |
| 7:33790522-33791432 | 7:34078379-34079848 | 7:35538150-35539052 |
| 7:35967810-35969055 | 7:36280133-36281043 | 7:36545347-36546249 |
| 7:36985740-36986642 | 7:41647630-41648532 | 7:41783296-41784251 |
| 7:43273927-43275127 | 7:43443846-43445974 | 7:44112090-44114352 |
| 7:44145527-44147967 | 7:44313454-44314408 | 7:44987535-44988449 |
| 7:45912932-45913902 | 7:47303307-47305685 | 7:47499715-47500686 |
| 7:47536431-47537429 | 7:47949034-47950274 | 7:48106918-48107898 |
| 7:48454957-48456103 | 7:50543717-50545695 | 7:50592195-50593416 |
| 7:50866434-50868050 | 7:51262468-51263445 | 7:55078916-55079832 |
| 7:55343769-55344751 | 7:56063635-56064553 | 7:64869868-64870936 |
| 7:65954010-65955948 | 7:66901587-66902575 | 7:69482331-69483326 |
| 7:70162290-70163209 | 7:70786819-70788515 | 7:70789303-70791442 |
| 7:73328649-73329616 | 7:73432351-73433253 | 7:73435128-73436033 |
| 7:73623988-73626005 | 7:73680588-73681490 | 7:73769741-73770738 |
| 7:73830878-73832527 | 7:74189923-74190838 | 7:74404724-74406810 |
| 7:74690460-74691385 | 7:75983305-75984279 | 7:75984444-75985437 |
| 7:75987988-75988976 | 7:76998859-76999761 | 7:77793550-77794646 |
| 7:80919798-80922904 | 7:86785825-86787468 | 7:97989609-97990588 |
| 7:98191197-98192698 | 7:98354081-98355036 | 7:98952247-98953920 |
| 7:98982857-98983815 | 7:99010692-99011658 | 7:99011450-99012928 |
| 7:99027565-99029267 | 7:99034509-99036261 | 7:99387592-99388530 |
| 7:99419310-99420298 | 7:99542693-99543600 | 7:100306694-100308231 |
| 7:100400165-100401148 | 7:100619938-100621635 | 7:100631691-100634582 |
| 7:100677894-100678887 | 7:100681184-100682722 | 7:100687502-100688811 |
| 7:100819252-100820226 | 7:100886354-100887572 | 7:100889489-100895292 |
| 7:101087126-101088051 | 7:101211143-101212136 | 7:101239766-101240720 |
| 7:102163671-102164578 | 7:102195590-102196579 | 7:102196628-102197732 |
| 7:102436133-102437051 | 7:102933206-102934529 | 7:103143073-103144239 |
| 7:106866578-106868960 | 7:107020334-107022254 | 7:108129954-108131763 |
| 7:114084261-114085249 | 7:117321851-117322815 | 7:117426689-117428384 |
| 7:120274073-120275969 | 7:127249901-127251448 | 7:128028003-128029989 |
| 7:128062503-128063478 | 7:128068473-128069437 | 7:128081291-128082353 |
| 7:128084669-128085571 | 7:128350348-128353086 | 7:128893045-128893947 |
| 7:128909836-128912240 | 7:128937071-128938022 | 7:129125737-129127970 |
| 7:129531881-129532874 | 7:130365498-130366452 | 7:130875971-130877284 |
| 7:131103095-131104317 | 7:132648171-132649277 | 7:133481845-133482747 |
| 7:135245477-135248438 | 7:135748153-135749493 | 7:137845903-137848610 |
| 7:138122148-138123114 | 7:138441421-138442384 | 7:138665183-138666085 |
| 7:138678027-138680553 | 7:138772648-138773550 | 7:138797163-138798101 |
| 7:138867519-138869163 | 7:138916649-138919447 | 7:139543916-139544818 |
| 7:139571069-139572015 | 7:140694973-140695875 | 7:141729955-141730857 |
| 7:141929461-141930876 | 7:142932975-142934655 | 7:143182761-143184190 |
| 7:143262241-143263747 | 7:143315271-143316564 | 7:148030444-148031346 |
| 7:148589923-148591332 | 7:149181773-149182710 | 7:149281470-149282438 |
| 7:149291715-149293855 | 7:149430759-149431745 | 7:149473149-149474102 |
| 7:149767297-149769170 | 7:150268852-150269753 | 7:150328925-150330362 |
| 7:150337960-150342841 | 7:150402009-150402996 | 7:150407073-150408018 |
| 7:150719117-150721456 | 7:150742477-150743860 | 7:150747845-150749874 |
| 7:150856574-150858748 | 7:151008143-151010676 | 7:151046983-151049743 |
| 7:151119438-151120370 | 7:151130934-151131916 | 7:151339990-151340900 |
| 7:151366865-151367836 | 7:151379381-151382900 | 7:151393974-151396603 |
| 7:151446920-151449468 | 7:151723983-151726282 | 7:151771179-151772688 |
| 7:151791946-151793454 | 7:152866678-152867658 | 7:154792615-154795076 |
| 7:154889271-154890222 | 7:155069770-155070769 | 7:155573058-155575289 |
| 7:155774581-155775557 | 7:155780510-155781505 | 7:155945810-155946788 |
| 7:156000547-156002674 | 7:156003922-156005370 | 7:156470401-156471810 |
| 7:156604477-156605379 | 7:156961352-156964325 | 7:156964687-156965589 |
| 7:157188548-157189461 | 7:157290141-157293383 | 7:157396508-157398570 |
| 7:157406386-157408008 | 7:157408381-157410259 | 7:157423368-157425318 |
| 7:157439958-157441985 | 7:157521114-157523104 | 7:157539311-157541437 |
| 7:157543629-157544601 | 7:157551906-157554079 | 7:157577091-157577993 |
| 7:157577994-157578922 | 7:157597787-157598775 | 7:157634422-157635540 |
| 7:157654015-157657167 | 7:157713077-157715521 | 7:157718963-157722679 |
| 7:157750367-157753025 | 7:157755701-157759955 | 7:157777747-157779304 |
| 7:157800745-157803234 | 7:157853423-157856513 | 7:157916632-157919404 |
| 7:158079261-158081878 | 7:158121287-158124877 | 7:158138953-158143876 |
| 7:158162459-158167867 | 7:158187604-158190427 | 7:158244526-158247949 |
| 7:158316115-158319182 | 7:158328961-158329899 | 7:158358930-158360513 |
| 7:158486917-158491349 | 7:158589990-158590963 | 7:158876615-158877517 |
| 7:158948586-158949565 | 7:159024375-159025363 | 7:159064510-159066984 |
| 7:159156133-159157684 | 8:631160-632384 | 8:638289-639342 |
| 8:642470-644439 | 8:676612-677785 | 8:693205-695352 |
| 8:718216-720027 | 8:742383-745390 | 8:749699-751898 |
| 8:805872-807633 | 8:903485-906378 | 8:963854-965082 |
| 8:965316-966373 | 8:1024285-1025761 | 8:1103570-1104472 |
| 8:1129607-1131217 | 8:1153711-1157814 | 8:1200443-1202043 |
| 8:1252008-1252970 | 8:1297857-1298778 | 8:1328318-1329546 |
| 8:1378726-1380903 | 8:1463013-1463950 | 8:1489204-1490135 |
| 8:1622018-1622991 | 8:1669165-1670136 | 8:1704498-1707205 |
| 8:1748820-1749807 | 8:1811171-1812318 | 8:1815623-1818664 |
| 8:1834143-1836361 | 8:1840953-1841855 | 8:1843311-1844274 |
| 8:1844083-1845053 | 8:1850012-1851795 | 8:1865256-1867454 |
| 8:1872535-1873773 | 8:1899758-1900660 | 8:1908482-1910213 |
| 8:1936394-1937947 | 8:1945265-1947284 | 8:1950420-1953097 |
| 8:1964414-1967432 | 8:2097962-2099631 | 8:2129524-2130466 |
| 8:2198783-2199930 | 8:2228364-2229817 | 8:2894763-2895798 |
| 8:4116872-4117819 | 8:4325598-4326540 | 8:6542984-6543949 |
| 8:6565860-6566789 | 8:6569344-6570277 | 8:6598397-6599381 |
| 8:6608735-6609679 | 8:6620577-6621541 | 8:7066468-7067370 |
| 8:8323981-8324902 | 8:8822890-8823851 | 8:8894319-8895239 |
| 8:9140413-9141863 | 8:10428221-10429171 | 8:10481404-10482381 |
| 8:10523740-10525551 | 8:10601842-10603068 | 8:10733667-10736162 |
| 8:10825205-10826129 | 8:11107476-11109232 | 8:11338132-11340012 |
| 8:11367832-11368817 | 8:11553428-11554421 | 8:11707483-11708469 |
| 8:11852860-11853825 | 8:17742525-17743478 | 8:18220728-18221742 |
| 8:19236634-19238419 | 8:20517283-20518203 | 8:20818101-20819061 |
| 8:21727175-21728145 | 8:21915697-21916599 | 8:22049960-22054132 |
| 8:22066818-22068109 | 8:22090564-22091466 | 8:22146863-22149059 |
| 8:22228404-22229351 | 8:22274352-22276036 | 8:22365501-22368070 |
| 8:22388542-22389503 | 8:22554368-22555364 | 8:22568225-22569172 |
| 8:22634736-22635899 | 8:22784336-22785632 | 8:22927194-22929312 |
| 8:23006041-23007425 | 8:23224912-23226590 | 8:23570660-23572893 |
| 8:25515450-25516352 | 8:25978073-25979777 | 8:26290041-26292315 |
| 8:27429985-27431339 | 8:27469282-27470278 | 8:27603737-27605731 |
| 8:27646477-27647433 | 8:27920737-27922686 | 8:28697914-28699606 |
| 8:28708993-28709944 | 8:28716898-28717866 | 8:28752918-28753888 |
| 8:30027243-30028230 | 8:32646139-32648266 | 8:33722848-33723783 |
| 8:37735277-37736198 | 8:40932392-40933983 | 8:41649563-41651778 |
| 8:41670946-41672169 | 8:41827530-41829606 | 8:41849447-41850429 |
| 8:42178731-42181152 | 8:42195939-42196874 | 8:42410246-42414401 |
| 8:42436744-42437708 | 8:42448672-42449774 | 8:42690994-42692273 |
| 8:43095545-43096535 | 8:47429974-47430876 | 8:47660615-47661517 |
| 8:47764008-47764981 | 8:47778040-47779008 | 8:48834698-48835600 |
| 8:48910051-48911044 | 8:49046081-49047066 | 8:49581570-49582710 |
| 8:51407171-51409148 | 8:52439134-52440221 | 8:52586489-52587470 |
| 8:55448823-55450148 | 8:55453903-55455981 | 8:56437261-56439018 |
| 8:60655323-60656225 | 8:60714203-60715154 | 8:60875972-60877882 |
| 8:60920874-60924940 | 8:61691051-61691953 | 8:66019967-66021245 |
| 8:68329154-68331236 | 8:73291109-73292044 | 8:74313765-74315772 |
| 8:81694761-81696534 | 8:82377663-82379205 | 8:85643386-85644373 |
| 8:94835633-94836564 | 8:95076967-95077869 | 8:95406109-95407011 |
| 8:96265813-96266715 | 8:97275319-97278842 | 8:98192105-98193083 |
| 8:99695866-99696800 | 8:101367884-101369384 | 8:101557703-101559220 |
| 8:102280988-102281926 | 8:106269638-106270617 | 8:107346700-107347638 |
| 8:108130906-108133097 | 8:108787015-108788560 | 8:113432856-113434142 |
| 8:118097733-118098636 | 8:120442502-120443404 | 8:120736574-120737476 |
| 8:122817908-122818823 | 8:123013243-123014192 | 8:123205811-123208467 |
| 8:123532187-123533593 | 8:125435404-125437054 | 8:125496200-125497152 |
| 8:127416103-127417035 | 8:127825149-127826060 | 8:127909729-127910711 |
| 8:128058363-128059277 | 8:129819458-129820360 | 8:133039580-133040526 |
| 8:133293529-133295249 | 8:133903657-133904559 | 8:138771266-138772749 |
| 8:138877225-138878192 | 8:139617402-139619539 | 8:139785809-139786803 |
| 8:139914014-139915533 | 8:139955535-139956524 | 8:140200189-140201187 |
| 8:140220951-140221878 | 8:140299901-140300807 | 8:140450508-140451484 |
| 8:140526432-140527550 | 8:140527376-140528368 | 8:140539933-140540879 |
| 8:140545708-140546799 | 8:140549096-140550268 | 8:140555360-140559305 |
| 8:140559913-140560860 | 8:141176173-141177314 | 8:141188204-141189112 |
| 8:141240689-141241654 | 8:141253429-141255177 | 8:141265099-141269007 |
| 8:141286571-141287972 | 8:141298556-141301478 | 8:141311256-141312158 |
| 8:141338523-141340265 | 8:141420839-141422717 | 8:141425048-141427963 |
| 8:141441728-141442860 | 8:141621801-141622722 | 8:141902422-141903782 |
| 8:142130619-142131554 | 8:142315380-142316331 | 8:142325024-142327137 |
| 8:142328907-142330082 | 8:142331098-142333827 | 8:142336952-142338653 |
| 8:142355684-142357152 | 8:142391968-142393929 | 8:142453553-142454455 |
| 8:142534693-142535656 | 8:142637411-142638405 | 8:142663332-142664324 |
| 8:142833844-142835994 | 8:143122177-143123617 | 8:143284416-143285924 |
| 8:143296450-143297402 | 8:143305239-143306204 | 8:143324869-143330192 |
| 8:143359250-143361591 | 8:143460810-143462322 | 8:143514190-143515173 |
| 8:143538620-143539606 | 8:143560390-143561387 | 8:143604806-143605785 |
| 8:143695216-143696184 | 8:143738007-143738988 | 8:143791560-143792555 |
| 8:143797179-143798118 | 8:143804639-143805563 | 8:143870669-143871970 |
| 8:143915652-143916563 | 8:143968584-143969565 | 8:143985913-143987911 |
| 8:144059424-144061541 | 8:144106868-144108666 | 8:144115757-144116722 |
| 8:144311974-144312944 | 8:144375059-144376395 | 8:144382122-144383778 |
| 8:144397512-144398485 | 8:144399673-144400630 | 8:144400518-144402080 |
| 8:144412107-144413008 | 8:144416047-144417617 | 8:144435593-144436581 |
| 8:144449924-144451077 | 8:144495738-144496690 | 8:144623038-144625044 |
| 8:144683629-144684715 | 8:144853433-144854404 | 8:144994008-144994909 |
| 9:471395-472355 | 9:2717080-2718373 | 9:2932617-2934387 |
| 9:4804098-4805324 | 9:5111081-5112500 | 9:7796897-7797799 |
| 9:13896577-13897507 | 9:14348299-14349868 | 9:19932130-19933061 |
| 9:34959595-34960510 | 9:35563521-35564492 | 9:35791803-35793391 |
| 9:37752559-37753520 | 9:69013440-69014429 | 9:74497442-74498418 |
| 9:74950713-74951687 | 9:87659012-87659914 | 9:87727253-87728155 |
| 9:89677835-89678815 | 9:90964772-90965757 | 9:91220399-91221377 |
| 9:91723920-91724880 | 9:91730396-91731390 | 9:91732069-91733536 |
| 9:93081248-93082186 | 9:93246982-93248535 | 9:93305271-93308046 |
| 9:94900377-94901280 | 9:94980239-94981185 | 9:95082068-95083046 |
| 9:95085114-95086985 | 9:96376593-96377521 | 9:96495995-96497481 |
| 9:96617299-96618712 | 9:96852417-96853319 | 9:97449739-97450641 |
| 9:98086878-98087848 | 9:98985286-98986239 | 9:100097841-100098792 |
| 9:100730725-100731679 | 9:101593886-101596349 | 9:101737016-101737968 |
| 9:105651815-105652717 | 9:108853635-108857241 | 9:109166929-109167911 |
| 9:111794317-111795746 | 9:113298127-113300461 | 9:113581750-113582652 |
| 9:113900642-113901632 | 9:114048410-114050513 | 9:114054818-114055805 |
| 9:114078050-114079351 | 9:114093937-114094842 | 9:120873696-120874598 |
| 9:121759478-121760457 | 9:121770207-121771164 | 9:122799600-122800854 |
| 9:123364296-123365276 | 9:123391540-123392682 | 9:124308744-124309698 |
| 9:124452842-124453839 | 9:125602747-125603649 | 9:125966083-125967064 |
| 9:126275805-126276791 | 9:126480821-126483490 | 9:127218744-127219709 |
| 9:127262929-127263834 | 9:127732716-127733676 | 9:127895469-127896371 |
| 9:128147878-128149964 | 9:128201551-128203090 | 9:128344903-128345861 |
| 9:128817284-128818226 | 9:129619152-129620134 | 9:129873617-129875329 |
| 9:130862317-130863285 | 9:130884043-130885020 | 9:130903456-130904627 |
| 9:131094510-131095412 | 9:131474394-131475389 | 9:131695771-131696672 |
| 9:131827978-131828969 | 9:131848403-131849374 | 9:131859241-131860783 |
| 9:132197422-132198372 | 9:132457141-132458571 | 9:133255558-133256894 |
| 9:133343346-133344332 | 9:133366877-133367858 | 9:133892371-133894322 |
| 9:133911896-133912846 | 9:133953826-133954755 | 9:134054014-134054976 |
| 9:134219017-134220419 | 9:134315127-134316097 | 9:134774813-134776037 |
| 9:134780668-134781643 | 9:134826458-134827681 | 9:134837812-134840576 |
| 9:135104813-135106002 | 9:135483298-135484200 | 9:135544866-135545768 |
| 9:135767082-135771012 | 9:135784246-135785938 | 9:135804116-135805048 |
| 9:135819559-135820473 | 9:135944519-135945650 | 9:135945649-135946608 |
| 9:136004652-136005554 | 9:136203782-136206728 | 9:136211280-136212222 |
| 9:136222170-136223146 | 9:136226400-136227663 | 9:136248405-136250752 |
| 9:136342463-136343436 | 9:136343488-136344394 | 9:136366432-136368761 |
| 9:136422981-136423945 | 9:136436447-136438184 | 9:136457140-136458131 |
| 9:136459250-136460244 | 9:136502522-136503514 | 9:136507892-136508874 |
| 9:136515092-136516084 | 9:136561543-136562624 | 9:136676640-136678277 |
| 9:136709191-136710159 | 9:136753910-136755091 | 9:136756682-136757631 |
| 9:136828651-136830367 | 9:136889966-136891390 | 9:136941980-136942953 |
| 9:137017583-137019805 | 9:137021901-137022856 | 9:137106124-137107088 |
| 9:137112153-137113120 | 9:137177502-137179980 | 9:137220298-137221293 |
| 9:137253030-137254551 | 9:137353464-137354456 | 9:137367015-137368591 |
| 9:137403012-137403957 | 9:137426148-137427076 | 9:137460224-137461397 |
| 9:137480595-137481840 | 9:137542054-137543861 | 9:138021077-138023520 |

### Table 2A—GBM

The DMRs may be those disclosed in Table 2 of GB2215319.1 and which are also available at: https://doi.org/10.5281/zenodo.10001252 as of 17 Oct. 2023. The entire contents of Table 2 of GB2215319.1 and https://doi.org/10.5281/zenodo.10001252 are expressly incorporated herein by reference as “Table 2A”.

TABLE 3
OAC/EAC

| | | |
|---|---|---|
| 1:873029-874226 | 1:970042-971582 | 1:1033060-1034469 |
| 1:1203638-1204819 | 1:1274637-1275814 | 1:1630373-1631897 |
| 1:1929639-1930905 | 1:1990413-1991524 | 1:2004672-2005825 |
| 1:2018375-2019576 | 1:2033089-2034203 | 1:2327121-2328219 |
| 1:2337896-2339021 | 1:2347434-2348593 | 1:2373137-2374221 |
| 1:2417699-2418847 | 1:2590295-2591528 | 1:2594949-2596104 |
| 1:2789010-2790206 | 1:2929839-2932405 | 1:3032467-3033693 |
| 1:3437281-3438439 | 1:3549519-3550709 | 1:3717760-3718952 |
| 1:3751894-3752938 | 1:3975901-3977047 | 1:3998259-3999388 |
| 1:4675441-4676700 | 1:4710545-4711722 | 1:4774309-4775820 |
| 1:5881327-5882504 | 1:6071548-6073302 | 1:6243538-6245025 |
| 1:6273285-6274394 | 1:6963214-6964384 | 1:7006671-7007787 |
| 1:7662309-7664656 | 1:7717603-7718744 | 1:7869215-7870268 |
| 1:7944597-7945533 | 1:8333320-8334468 | 1:9416989-9418143 |
| 1:9542135-9543109 | 1:9671302-9672461 | 1:9699748-9700856 |
| 1:10920560-10921707 | 1:11519846-11521017 | 1:11550194-11551326 |
| 1:11715863-11717020 | 1:11779415-11780559 | 1:12062630-12064454 |
| 1:12252956-12253995 | 1:12793813-12794838 | 1:13512079-13514817 |
| 1:13516481-13517957 | 1:13559675-13560951 | 1:14848224-14849385 |
| 1:15337235-15338895 | 1:15340064-15341095 | 1:16432651-16433687 |
| 1:16520134-16521317 | 1:16545469-16546638 | 1:17754728-17755959 |
| 1:19155834-19156833 | 1:20342631-20344070 | 1:20472734-20474021 |
| 1:20570991-20572083 | 1:20637416-20638579 | 1:21567445-21568639 |
| 1:22288825-22290007 | 1:22339966-22340997 | 1:22345540-22346705 |
| 1:22611150-22612271 | 1:22952610-22954170 | 1:23948827-23949951 |
| 1:24928712-24932439 | 1:25617435-25618853 | 1:26045388-26047390 |
| 1:26050957-26051962 | 1:26351108-26352110 | 1:29122851-29124041 |
| 1:29223366-29224407 | 1:29478192-29479308 | 1:30342266-30343454 |
| 1:30764756-30765909 | 1:30776504-30777618 | 1:31181358-31182545 |
| 1:32873153-32874323 | 1:33512246-33513219 | 1:34175472-34176648 |
| 1:34176711-34177974 | 1:34752754-34753706 | 1:36087375-36088556 |
| 1:36438513-36439522 | 1:36483383-36484513 | 1:36751224-36752143 |
| 1:37201621-37202564 | 1:37753535-37754703 | 1:37764516-37765727 |
| 1:38123746-38124892 | 1:38475613-38477209 | 1:38558985-38560141 |
| 1:38803886-38805074 | 1:39526606-39527733 | 1:39571470-39572592 |
| 1:39638846-39640230 | 1:39683402-39684765 | 1:40883740-40884940 |
| 1:41321977-41323147 | 1:41938492-41939508 | 1:41955432-41956625 |
| 1:42784641-42786182 | 1:42829420-42830322 | 1:42972715-42973824 |
| 1:43197073-43198121 | 1:44009278-44010402 | 1:44117763-44119423 |
| 1:44524272-44525252 | 1:46447552-46449450 | 1:46490223-46491748 |
| 1:46543323-46545021 | 1:47443659-47446867 | 1:48204282-48205274 |
| 1:49256121-49257250 | 1:50422437-50425605 | 1:51644261-51645249 |
| 1:52666381-52667283 | 1:52842380-52844169 | 1:53061289-53063129 |
| 1:53463603-53464718 | 1:53599423-53600439 | 1:54132445-54133578 |
| 1:55155079-55156162 | 1:56363043-56364087 | 1:57877798-57878700 |
| 1:60073151-60074582 | 1:62194298-62195551 | 1:62848707-62849745 |
| 1:63318596-63321202 | 1:63321032-63322181 | 1:63322151-63325250 |
| 1:63329374-63331150 | 1:64691128-64692307 | 1:65264205-65266786 |
| 1:65554477-65555629 | 1:66050872-66051981 | 1:66286921-66287999 |
| 1:66751394-66753140 | 1:67134413-67135731 | 1:68450545-68451642 |
| 1:69566642-69570381 | 1:69759469-69760507 | 1:69918856-69920014 |
| 1:75127211-75129031 | 1:75129214-75136180 | 1:77801653-77802674 |
| 1:82774834-82775917 | 1:87151011-87152184 | 1:88892251-88893288 |
| 1:89803667-89804710 | 1:90716090-90720470 | 1:90723853-90727491 |
| 1:90728547-90731448 | 1:90834068-90835462 | 1:90835339-90836946 |
| 1:92028812-92030004 | 1:92479983-92482917 | 1:93566786-93567893 |
| 1:94608079-94609270 | 1:101313619-101314735 | 1:101846495-101847588 |
| 1:107055040-107056141 | 1:109667088-109668891 | 1:109669946-109670940 |
| 1:110129696-110130890 | 1:110501888-110502865 | 1:110603919-110605040 |
| 1:110674187-110676214 | 1:111234438-111235474 | 1:111270489-111272186 |
| 1:112747762-112748920 | 1:113226068-113227225 | 1:114151741-114155031 |
| 1:114214666-114215666 | 1:114323961-114324979 | 1:114345559-114346746 |
| 1:115281213-115282351 | 1:115320028-115321056 | 1:115337159-115339207 |
| 1:115827965-115829439 | 1:116741627-116742739 | 1:116747764-116748687 |
| 1:118978778-118980800 | 1:118983912-118985082 | 1:118984942-118986583 |
| 1:118986719-118989830 | 1:118992385-118994259 | 1:118998942-119001417 |
| 1:119005682-119009250 | 1:143660544-143661720 | 1:145994112-145995206 |
| 1:147050333-147051526 | 1:147072125-147073684 | 1:147077790-147081133 |
| 1:147696751-147697823 | 1:147773766-147774747 | 1:150150293-150151462 |
| 1:150980569-150981822 | 1:151130796-151132301 | 1:151145345-151146529 |
| 1:152107400-152108587 | 1:152109884-152111014 | 1:152513550-152516371 |
| 1:153057587-153058569 | 1:153223453-153224472 | 1:154139258-154140387 |
| 1:154331226-154332227 | 1:154769845-154770846 | 1:154771681-154772838 |
| 1:155141860-155143291 | 1:155320147-155321724 | 1:156300011-156301612 |
| 1:156338879-156340010 | 1:156844527-156846333 | 1:156848527-156849850 |
| 1:156926957-156928119 | 1:157100833-157101850 | 1:157109639-157110818 |
| 1:157928823-157929805 | 1:157978493-157979692 | 1:158115072-158116157 |
| 1:158149154-158150447 | 1:158182967-158184486 | 1:158656895-158657885 |
| 1:158699883-158701278 | 1:158765738-158766973 | 1:158974509-158975469 |
| 1:159313237-159315046 | 1:159777932-159778984 | 1:160083539-160084742 |
| 1:160127136-160128241 | 1:160138782-160139895 | 1:160170815-160171882 |
| 1:161140800-161141883 | 1:161684692-161685785 | 1:161973196-161974323 |
| 1:161983285-161984614 | 1:163422241-163423203 | 1:164320371-164322137 |
| 1:164853157-164854291 | 1:165234702-165236807 | 1:165354309-165357445 |
| 1:166154732-166156241 | 1:166920454-166922001 | 1:166988378-166989676 |
| 1:170522314-170523353 | 1:170660664-170662875 | 1:171666702-171667804 |
| 1:173095412-173096331 | 1:173257344-173258246 | 1:173392844-173393888 |
| 1:173441726-173442732 | 1:173562542-173563615 | 1:175044685-175045813 |
| 1:175598664-175600157 | 1:176690648-176691562 | 1:180231582-180234229 |
| 1:181482174-181484302 | 1:181730826-181732012 | 1:182069656-182070795 |
| 1:182128209-182129355 | 1:184588827-184589989 | 1:186041829-186042929 |
| 1:191614771-191615947 | 1:193395283-193396362 | 1:197840796-197841866 |
| 1:197910034-197911223 | 1:197912052-197914269 | 1:197917714-197920703 |
| 1:197920878-197922078 | 1:198982898-198984002 | 1:199462695-199463734 |
| 1:199857929-199858903 | 1:200037862-200039089 | 1:200873026-200874344 |
| 1:201223546-201224647 | 1:201283114-201284296 | 1:201493819-201495006 |
| 1:201622753-201623828 | 1:201894778-201895972 | 1:201953938-201955123 |
| 1:202342297-202343443 | 1:203044924-203046022 | 1:203074998-203076700 |
| 1:203099808-203100710 | 1:203230127-203231631 | 1:204138499-204139411 |
| 1:204662045-204662971 | 1:204979923-204980975 | 1:204984977-204986463 |
| 1:204996479-204997703 | 1:205042661-205043916 | 1:205057279-205059599 |
| 1:205068729-205070538 | 1:205231868-205232876 | 1:205616060-205616969 |
| 1:206798173-206799380 | 1:207305140-207306211 | 1:207668780-207670329 |
| 1:207676811-207677960 | 1:207958493-207960038 | 1:208778291-208779193 |
| 1:209172665-209173840 | 1:209256391-209257568 | 1:209423821-209424945 |
| 1:209825993-209827056 | 1:210325221-210326247 | 1:211861044-211862048 |
| 1:212623757-212625458 | 1:212703332-212704458 | 1:213982312-213984207 |
| 1:213984638-213985984 | 1:214224603-214225505 | 1:217136957-217139210 |
| 1:220091000-220092022 | 1:220531735-220532757 | 1:220680542-220681683 |
| 1:220876580-220879384 | 1:220879653-220882944 | 1:220887130-220888325 |
| 1:220991197-220992199 | 1:221783674-221784702 | 1:222369691-222370802 |
| 1:223083530-223084724 | 1:223664948-223666028 | 1:223666294-223667311 |
| 1:223697550-223698529 | 1:224063866-224064901 | 1:224701038-224702173 |
| 1:224702775-224703809 | 1:225886201-225887956 | 1:225974902-225976100 |
| 1:226679730-226680818 | 1:228280324-228281521 | 1:228315994-228318268 |
| 1:228469116-228470377 | 1:229165252-229166388 | 1:229431909-229433105 |
| 1:229433276-229435387 | 1:230321410-230323968 | 1:230335751-230336761 |
| 1:230393483-230394624 | 1:231160621-231164248 | 1:231188518-231189606 |
| 1:231622529-231623566 | 1:231845648-231846611 | 1:234156821-234157995 |
| 1:235648940-235651490 | 1:235992706-235993847 | 1:236168122-236169294 |
| 1:236735002-236736961 | 1:236738843-236739945 | 1:237920092-237921130 |
| 1:241944436-241945528 | 1:241956885-241959092 | 1:243589830-243591603 |
| 1:243932288-243933310 | 1:244185923-244187117 | 1:244377881-244378920 |
| 1:244583300-244584472 | 1:244636608-244637756 | 1:245563767-245564848 |
| 1:246032940-246034018 | 1:247107355-247108523 | 1:247280487-247281535 |
| 1:247347460-247348746 | 1:247531174-247532595 | 1:247841033-247842973 |
| 1:247910950-247912210 | 1:247919269-247920512 | 10:725088-726743 |
| 10:731581-733299 | 10:1209843-1211201 | 10:1216235-1217550 |
| 10:1274258-1275929 | 10:1395890-1397076 | 10:1503958-1505132 |
| 10:1517692-1519000 | 10:1521934-1523489 | 10:1566631-1568071 |
| 10:1625849-1627733 | 10:1963046-1964245 | 10:2012812-2013863 |
| 10:2082676-2083830 | 10:2098970-2100123 | 10:2104721-2105905 |
| 10:2410254-2411457 | 10:3141129-3142166 | 10:3287044-3288304 |
| 10:3511673-3512821 | 10:3537928-3539536 | 10:3940768-3941950 |
| 10:4148822-4150194 | 10:4315845-4316902 | 10:6025417-6026465 |
| 10:6063096-6064204 | 10:6486925-6487928 | 10:6617392-6618417 |
| 10:7096862-7098006 | 10:7169554-7171851 | 10:7171609-7173896 |
| 10:7180276-7181403 | 10:7287863-7289040 | 10:7411986-7413965 |
| 10:7521910-7523092 | 10:7551938-7552975 | 10:7575073-7576170 |
| 10:7721199-7722383 | 10:7722176-7723329 | 10:7733474-7734760 |
| 10:7791104-7792112 | 10:8033724-8034906 | 10:8035295-8036900 |
| 10:8036658-8038374 | 10:8046353-8047405 | 10:8050690-8053316 |
| 10:8146419-8147539 | 10:10931091-10932103 | 10:10951498-10952776 |
| 10:11043252-11044359 | 10:11074971-11076094 | 10:11091395-11092447 |
| 10:11164342-11166598 | 10:11304915-11305963 | 10:11339296-11340479 |
| 10:11343542-11344734 | 10:11434293-11435378 | 10:11440655-11441950 |
| 10:11551129-11552162 | 10:11744428-11745481 | 10:12512502-12513531 |
| 10:12614424-12615602 | 10:12638175-12639334 | 10:12643953-12645110 |
| 10:12709716-12710829 | 10:12800111-12801296 | 10:12818921-12820057 |
| 10:12924326-12925436 | 10:12925278-12926180 | 10:13859341-13860434 |
| 10:14994802-14995965 | 10:15145919-15146945 | 10:17130345-17131247 |
| 10:17226455-17227483 | 10:18290265-18291404 | 10:19633730-19634824 |
| 10:21146154-21147445 | 10:21285440-21286470 | 10:21387353-21388549 |
| 10:21493819-21495014 | 10:22344906-22346267 | 10:22599095-22600169 |
| 10:22655458-22656495 | 10:23172587-23175586 | 10:23189967-23191847 |
| 10:23194495-23196412 | 10:23345733-23346773 | 10:24552459-24553644 |
| 10:25933411-25934967 | 10:26211228-26212448 | 10:26642414-26643579 |
| 10:27383292-27384258 | 10:27742441-27747290 | 10:28238390-28239456 |
| 10:28430626-28431646 | 10:28883419-28884528 | 10:31070854-31071847 |
| 10:31319132-31321615 | 10:33106396-33107451 | 10:34426637-34427826 |
| 10:35607140-35608606 | 10:42366856-42368597 | 10:42543403-42544390 |
| 10:43668274-43669379 | 10:43938478-43939553 | 10:43955078-43956029 |
| 10:44919685-44921217 | 10:45205107-45206712 | 10:46330920-46331957 |
| 10:48502414-48503499 | 10:48604230-48605484 | 10:48698107-48699242 |
| 10:48938241-48939430 | 10:48982295-48983969 | 10:49109294-49110431 |
| 10:49112593-49113522 | 10:49279678-49280708 | 10:49282766-49283761 |
| 10:49297356-49298373 | 10:49395526-49397052 | 10:49396986-49399217 |
| 10:49608357-49611464 | 10:49612516-49615204 | 10:49678368-49680176 |
| 10:50402778-50404101 | 10:55629406-55632084 | 10:60388750-60390022 |
| 10:61001455-61002626 | 10:61409785-61410945 | 10:62817769-62819750 |
| 10:63697663-63698608 | 10:66927598-66928989 | 10:67098374-67099505 |
| 10:68560897-68562410 | 10:69052201-69053280 | 10:69070451-69071440 |
| 10:69567405-69568556 | 10:69644981-69646042 | 10:69822991-69824466 |
| 10:69929280-69930958 | 10:70598354-70601094 | 10:70751739-70752870 |
| 10:70992881-70993908 | 10:70998364-70999398 | 10:71399391-71401034 |
| 10:71443540-71444690 | 10:71444881-71446027 | 10:71474071-71475207 |
| 10:71511971-71513104 | 10:71602211-71603202 | 10:71676992-71678142 |
| 10:74589003-74590170 | 10:74750539-74751588 | 10:74816354-74817406 |
| 10:74956721-74957830 | 10:75046571-75047636 | 10:75433733-75434928 |
| 10:76443950-76444916 | 10:76875202-76876246 | 10:76972806-76973931 |
| 10:76974399-76975560 | 10:77109958-77111028 | 10:77427900-77428947 |
| 10:77616577-77617555 | 10:77636228-77639302 | 10:77865872-77866979 |
| 10:78277744-78278676 | 10:78735448-78736612 | 10:78749979-78751121 |
| 10:79393817-79394968 | 10:80024646-80025681 | 10:80278342-80279303 |
| 10:80342834-80343988 | 10:80355862-80358099 | 10:80372653-80373621 |
| 10:84404031-84405035 | 10:86181612-86182726 | 10:86262399-86264011 |
| 10:86289213-86290201 | 10:86364588-86368185 | 10:86553115-86554220 |
| 10:87742925-87743969 | 10:87845619-87846661 | 10:88499649-88500606 |
| 10:89091479-89092625 | 10:89564070-89565180 | 10:89837282-89838833 |
| 10:92419665-92421393 | 10:93060578-93064022 | 10:93065189-93066890 |
| 10:93556237-93557244 | 10:95307639-95308721 | 10:95552025-95553112 |
| 10:96118229-96119325 | 10:96396800-96397843 | 10:97028567-97029660 |
| 10:99235735-99237638 | 10:99296656-99297814 | 10:99526707-99528680 |
| 10:99537246- | 10:100658880- | 10:100708943- |
| 99538414 | 100660436 | 100710136 |
| 10:100714982-100716371 | 10:100740991- | 10:100747325- |
| | 100742181 | 100749869 |
| 10:100825810- | 10:100829551- | 10:101130533- |
| 100827053 | 100831137 | 101133350 |
| 10:101135764- | 10:101138948- | 10:101145705- |
| 101137647 | 101141314 | 101147449 |
| 10:101224041- | 10:101226906- | 10:101235838- |
| 101226782 | 101230471 | 101238923 |
| 10:101682838- | 10:101774212- | 10:101775342- |
| 101683860 | 101775304 | 101777190 |
| 10:102240495- | 10:102646722- | 10:102648738- |
| 102241788 | 102647792 | 102649723 |
| 10:103276171- | 10:103691996- | 10:104433245- |
| 103278344 | 103693192 | 104434357 |
| 10:104485240- | 10:104589061- | 10:107163462- |
| 104486337 | 104590091 | 107165397 |
| 10:107914608- | 10:110340424- | 10:110481180- |
| 107915884 | 110341505 | 110482230 |
| 10:110803321- | 10:110826431- | 10:112526650- |
| 110804345 | 110827519 | 112527728 |
| 10:112601075- | 10:112912121- | 10:113518910- |
| 112602117 | 112913229 | 113519988 |
| 10:113593841- | 10:114044607- | 10:116270864- |
| 113594951 | 114045809 | 116275113 |
| 10:116669469- | 10:116740760- | 10:116785864- |
| 116670745 | 116741946 | 116786985 |
| 10:116830984- | 10:117131571- | 10:117165272- |
| 116832086 | 117133578 | 117167558 |
| 10:117216003- | 10:117531986- | 10:117540893- |
| 117217196 | 117537835 | 117546406 |
| 10:117547587- | 10:117550295- | 10:117551350- |
| 117548926 | 117551374 | 117554164 |
| 10:117734399- | 10:117738806- | 10:118481565- |
| 117735758 | 117739954 | 118482504 |
| 10:118593970- | 10:118786604- | 10:118821767- |
| 118595929 | 118787621 | 118822922 |
| 10:120581090- | 10:120818127- | 10:121610116- |
| 120582209 | 120819253 | 121611235 |
| 10:121987416- | 10:123133042- | 10:123141791- |
| 121988462 | 123138232 | 123144369 |
| 10:123147342- | 10:123397122- | 10:123493771- |
| 123151056 | 123398273 | 123494733 |
| 10:123665188- | 10:123677741- | 10:123972255- |
| 123667113 | 123678664 | 123973833 |
| 10:124098476- | 10:124263832- | 10:124447057- |
| 124100330 | 124264974 | 124448736 |
| 10:124550241- | 10:125049869- | 10:125786556- |
| 124551425 | 125051054 | 125787679 |
| 10:125994805- | 10:126100632- | 10:126386489- |
| 125995809 | 126101772 | 126389569 |
| 10:126392013- | 10:127735379- | 10:128121503- |
| 126393072 | 127738738 | 128122470 |
| 10:128315358- | 10:128325523- | 10:128384652- |
| 128316344 | 128326966 | 128385845 |
| 10:128571802- | 10:128577735- | 10:128708885- |
| 128572897 | 128578759 | 128710982 |
| 10:128720988- | 10:128958473- | 10:129021900- |
| 128722083 | 128959648 | 129023033 |
| 10:129352787- | 10:129391609- | 10:129473733- |
| 129353948 | 129392715 | 129474858 |
| 10:129742776- | 10:129743815- | 10:129778773- |
| 129743908 | 129744950 | 129779928 |
| 10:129787717- | 10:129790312- | 10:129794033- |
| 129788765 | 129791430 | 129795172 |
| 10:129837552- | 10:129841447- | 10:129866778- |
| 129838933 | 129842721 | 129869340 |
| 10:129872096- | 10:129902426- | 10:129958330- |
| 129874036 | 129903554 | 129961112 |
| 10:129961937- | 10:129968018- | 10:130125140- |
| 129968103 | 129973954 | 130126328 |
| 10:130232591- | 10:131014145- | 10:131074911- |
| 130233756 | 131015047 | 131075984 |
| 10:131200876- | 10:131233297- | 10:131310238- |
| 131202740 | 131234389 | 131311328 |
| 10:131311381- | 10:131375988- | 10:131453468- |
| 131313068 | 131377040 | 131454669 |
| 10:131773358- | 10:131843445- | 10:131983259- |
| 131774678 | 131845432 | 131984359 |
| 10:132044293- | 10:132060734- | 10:132062704- |
| 132045813 | 132061870 | 132063812 |
| 10:132103273- | 10:132115422- | 10:132137530- |
| 132105344 | 132116805 | 132138856 |
| 10:132591389- | 10:132625705- | 10:132784258- |
| 132592564 | 132626886 | 132787563 |
| 10:132827784- | 10:132868813- | 10:132964076- |
| 132828952 | 132870389 | 132965233 |
| 10:132987716- | 10:132989069- | 10:133170303- |
| 132988842 | 132990785 | 133171373 |
| 10:133219484- | 10:133223270- | 10:133229396- |
| 133220685 | 133224431 | 133231938 |
| 10:133235895- | 10:133304461- | 10:133310010- |
| 133237995 | 133305633 | 133311124 |
| 11:375513-376694 | 11:379470-381193 | 11:504196-505384 |
| 11:525841-526934 | 11:527674-528828 | 11:628091-629195 |
| 11:1373567-1374612 | 11:1651788-1652953 | 11:1748244-1749429 |
| 11:1901308-1902838 | 11:2087860-2088965 | 11:2139049-2141837 |
| 11:2143605-2145010 | 11:2547297-2548844 | 11:2805064-2806135 |
| 11:2868769-2870462 | 11:3659470-3660552 | 11:4211683-4212860 |
| 11:4367576-4368629 | 11:4488685-4489721 | 11:4586586-4587717 |
| 11:4698147-4699084 | 11:5250087-5251334 | 11:5302444-5303597 |
| 11:5351263-5352165 | 11:5505676-5506689 | 11:5690325-5691479 |
| 11:5820007-5820961 | 11:5884211-5885242 | 11:5929055-5930168 |
| 11:5934993-5936149 | 11:6156361-6157403 | 11:6640510-6641686 |
| 11:6783118-6784209 | 11:8061947-8063537 | 11:8080216-8082130 |
| 11:8102896-8104012 | 11:8230931-8232095 | 11:8315253-8316511 |
| 11:10633820-10634918 | 11:11382704-11383796 | 11:12162985-12164055 |
| 11:14971510-14972700 | 11:17404909-17405999 | 11:17475548-17477668 |
| 11:17631603-17632687 | 11:17695196-17696670 | 11:17720621-17722133 |
| 11:17776763-17777953 | 11:18011248-18012313 | 11:18634752-18635914 |
| 11:18790517-18793067 | 11:19345038-19346669 | 11:19713134-19715158 |
| 11:20158209-20159751 | 11:20159982-20161718 | 11:20207412-20209050 |
| 11:20342334-20343473 | 11:20595843-20599329 | 11:20603986-20606012 |
| 11:20670608-20672053 | 11:22342061-22343328 | 11:22343283-22344463 |
| 11:22432094-22433705 | 11:27522625-27523723 | 11:27718730-27721358 |
| 11:30015611-30018125 | 11:30535479-30536736 | 11:30579842-30580934 |
| 11:30583647-30587189 | 11:31797088-31801282 | 11:31801109-31802218 |
| 11:31802581-31806943 | 11:31813423-31821019 | 11:31823252-31824574 |
| 11:31824306-31828227 | 11:31987021-31988200 | 11:32425947-32429711 |
| 11:32430446-32431612 | 11:32432584-32438791 | 11:32438659-32440235 |
| 11:33949389-33950514 | 11:34075812-34076857 | 11:35348795-35349821 |
| 11:36150563-36151559 | 11:36514692-36515744 | 1 :43546924-43548288 |
| 11:43577244-43583257 | 11:44302878-44305559 | 11:44308754-44312211 |
| 11:44424228-44425260 | 11:44919251-44920406 | 11:45235197-45236099 |
| 11:45695242-45697182 | 11:45700307-45701710 | 11:45720807-45722923 |
| 11:46144290-46145413 | 11:47943568-47944668 | 11:49207884-49209125 |
| 11:57346951-57347996 | 11:57482406-57483892 | 11:57646406-57647716 |
| 11:59060134-59061253 | 11:60700682-60701776 | 11:61341866-61342971 |
| 11:61515942-61517096 | 11:61826891-61829533 | 11:61857327-61858463 |
| 11:61955065-61956252 | 11:62017030-62017998 | 11:62207687-62209606 |
| 11:62926030-62927209 | 11:63228878-63229866 | 11:63462538-63463631 |
| 11:63609936-63610984 | 11:64060644-64061795 | 11:64289848-64290832 |
| 11:64428964-64430098 | 11:64453529-64454566 | 11:64532355-64533359 |
| 11:64660316-64661437 | 11:64971275-64972873 | 11:65169648-65170793 |
| 11:65641715-65642948 | 11:65726830-65727967 | 11:65832716-65834530 |
| 11:65867213-65869038 | 11:66419468-66421551 | 11:66558718-66559893 |
| 11:67521513-67522610 | 11:67674672-67675710 | 11:68329178-68330319 |
| 11:68409394-68410567 | 11:68617140-68619040 | 11:68661567-68662669 |
| 11:68668704-68669940 | 11:68854119-68855382 | 11:68877708-68878762 |
| 11:69013251-69014411 | 11:69018898-69019800 | 11:69424973-69426018 |
| 11:69658375-69659519 | 11:69711851-69712809 | 11:69773798-69774954 |
| 11:69818589-69820456 | 11:70233295-70234481 | 11:71608868-71610022 |
| 11:72215081-72216289 | 11:72583892-72585272 | 11:72589692-72591230 |
| 11:73168868-73169961 | 11:73304692-73305813 | 11:73963389-73964497 |
| 11:75243124-75244026 | 11:75279007-75279943 | 11:76410738-76411837 |
| 11:77587782-77589254 | 11:78023430-78024606 | 11:78595269-78596422 |
| 11:78907595-78909066 | 11:79068752-79070251 | 11:82732343-82734614 |
| 11:83219692-83220750 | 11:85923730-85924695 | 11:89490560-89492019 |
| 11:90133766-90135400 | 11:90220912-90222102 | 11:92843685-92845095 |
| 11:92968858-92969965 | 11:94400467-94402132 | 11:94762132-94763162 |
| 11:96037839-96038882 | 11:99069532-99070677 | 11:101582034- |
| | | 101584615 |
| 11:102466594- | 11:102468346- | 11:105609466- |
| 102467644 | 102469464 | 105611712 |
| 11:109301509- | 11:109891795- | 11:110133764- |
| 109302603 | 109892837 | 110134892 |
| 11:110851438- | 11:111538593- | 11:111539757- |
| 110852627 | 111539588 | 111542016 |
| 11:112480527- | 11:112776276- | 11:113184164- |
| 112481511 | 112777299 | 113185169 |
| 11:113387442- | 11:114219976- | 11:114236612- |
| 113388595 | 114220982 | 114237714 |
| 11:114267514- | 11:114356562- | 11:114366097- |
| 114268624 | 114357562 | 114367287 |
| 11:115506970- | 11:115659045- | 11:115671989- |
| 115508042 | 115660909 | 115673181 |
| 11:115870766- | 11:116276095- | 11:116580708- |
| 115871775 | 116277454 | 116581879 |
| 11:117505005- | 11:117523962- | 11:117601049- |
| 117506207 | 117525000 | 117602002 |
| 11:117781001- | 11:117795555- | 11:117918251- |
| 117782023 | 117796780 | 117919412 |
| 11:117933525- | 11:118304218- | 11:120467318- |
| 117934633 | 118305142 | 120468348 |
| 11:120563631- | 11:121087285- | 11:121128890- |
| 120565839 | 121088276 | 121130607 |
| 11:121160022- | 11:122099822- | 11:122101184- |
| 121161051 | 122100975 | 122102347 |
| 11:122293277- | 11:122368597- | 11:122448403- |
| 122294279 | 122369714 | 122449554 |
| 11:122881777- | 11:122978834- | 11:123010923- |
| 122882823 | 122980864 | 123011956 |
| 11:123163328- | 11:123166634- | 11:123426044- |
| 123164376 | 123167759 | 123427200 |
| 11:123573378- | 11:124868373- | 11:124919960- |
| 123574379 | 124869706 | 124922156 |
| 11:124924337- | 11:125262850- | 11:125441910- |
| 124925488 | 125264239 | 125442882 |
| 11:125463059- | 11:125494264- | 11:125885239- |
| 125465217 | 125495704 | 125886306 |
| 11:128643218- | 11:128685353- | 11:128866498- |
| 128644254 | 128687566 | 128867546 |
| 11:128951962- | 11:129366950- | 11:130368887- |
| 128952964 | 129368002 | 130370021 |
| 11:130472812- | 11:131371114- | 11:131498563- |
| 130474007 | 131372300 | 131499623 |
| 11:131686923- | 11:132126017- | 11:132283023- |
| 131688717 | 132127424 | 132283975 |
| 11:132307218- | 11:132656482- | 11:132942499- |
| 132308305 | 132657851 | 132945117 |
| 11:132993652- | 11:133076211- | 11:133082419- |
| 132995037 | 133077546 | 133084052 |
| 11:133227991- | 11:133500691- | 11:133829313- |
| 133229115 | 133501709 | 133830455 |
| 11:134139948- | 11:134609587- | 11:134655829- |
| 134141138 | 134610771 | 134657706 |
| 11:134712908- | 12:224500-225831 | 12:263202-264362 |
| 134714055 | | |
| 12:453081-454444 | 12:683628-684791 | 12:1434272-1435469 |
| 12:1863580-1864885 | 12:1871121-1872244 | 12:1909131-1910289 |
| 12:1928279-1929459 | 12:2046559-2048747 | 12:2053804-2054945 |
| 12:2111555-2112658 | 12:2269042-2270105 | 12:2376879-2378006 |
| 12:2403549-2404656 | 12:2503786-2505403 | 12:2752376-2753560 |
| 12:2771096-2772670 | 12:3199498-3200694 | 12:3289751-3290872 |
| 12:3304691-3305858 | 12:3367284-3368346 | 12:3803249-3804354 |
| 12:3944938-3945925 | 12:4372406-4374183 | 12:4433587-4434734 |
| 12:4443848-4444855 | 12:4765704-4766850 | 12:5043022-5044461 |
| 12:5045058-5046684 | 12:5070832-5071868 | 12:5429971-5431841 |
| 12:5944856-5946168 | 12:6033235-6034317 | 12:7368918-7370101 |
| 12:7486243-7487249 | 12:7504349-7505476 | 12:7705435-7706755 |
| 12:7712489-7713751 | 12:7873318-7874370 | 12:8563065-8564171 |
| 12:8833941-8834843 | 12:8842390-8843526 | 12:9126359-9127429 |
| 12:9203169-9204252 | 12:9377689-9378779 | 12:9380919-9381951 |
| 12:9910160-9911204 | 12:9947954-9949009 | 12:10178730-10179882 |
| 12:10189830-10190755 | 12:11016142-11017171 | 12:12908445-12909564 |
| 12:13114320-13115483 | 12:14916863-14917916 | 12:15321731-15323628 |
| 12:15620913-15621863 | 12:16604452-16605703 | 12:19396271-19397358 |
| 12:19940855-19941935 | 12:21436223-21437187 | 12:21568340-21569417 |
| 12:21765618-21766772 | 12:21940457-21942994 | 12:22071173-22072281 |
| 12:22375107-22376273 | 12:22924851-22925799 | 12:24663752-24664930 |
| 12:24747926-24748950 | 12:24901887-24903762 | 12:24917546-24918513 |
| 12:25031845-25032921 | 12:25779734-25781333 | 12:28189414-28190529 |
| 12:30169730-30170909 | 12:30200371-30201443 | 12:30320066-30321342 |
| 12:32444246-32445429 | 12:33437977-33440693 | 12:34363429-34364600 |
| 12:34364410-34366217 | 12:34377275-34378454 | 12:40691648-40694050 |
| 12:41312437-41313432 | 12:41573013-41574161 | 12:41905075-41906136 |
| 12:43551279-43553053 | 12:44294630-44295594 | 12:45049484-45051912 |
| 12:46258618-46259672 | 12:46830438-46833053 | 12:46934309-46935490 |
| 12:48183345-48185376 | 12:48526694-48529208 | 12:49263303-49264399 |
| 12:49294836-49296274 | 12:49335674-49337028 | 12:50032346-50033533 |
| 12:50248069-50249177 | 12:51588779-51590293 | 12:51868584-51869759 |
| 12:51906528-51907733 | 12:51986615-51987700 | 12:52006521-52008030 |
| 12:52651835-52653297 | 12:52714916-52716074 | 12:52964958-52966278 |
| 12:53676839-53677967 | 12:53930123-53931286 | 12:53935476-53936744 |
| 12:53938608-53939807 | 12:53950832-53952665 | 12:53953422-53954499 |
| 12:53965369-53967783 | 12:53968742-53970469 | 12:54004490-54006299 |
| 12:54421896-54422922 | 12:54460014-54461071 | 12:54518247-54519351 |
| 12:54559974-54561134 | 12:54574074-54575187 | 12:54609695-54611674 |
| 12:54634995-54636446 | 12:55732688-55733859 | 12:57620328-57621857 |
| 12:57627558-57628704 | 12:57668487-57669633 | 12:57670404-57671334 |
| 12:57818188-57819383 | 12:58172184-58173365 | 12:62145260-62146580 |
| 12:63149471-63152092 | 12:64823788-64826969 | 12:65120648-65122160 |
| 12:66584296-66585286 | 12:66765977-66766879 | 12:68252784-68254261 |
| 12:68959204-68960368 | 12:70755287-70756306 | 12:72270971-72274456 |
| 12:75207465-75208713 | 12:75208719-75210256 | 12:80707671-80708930 |
| 12:80713626-80714853 | 12:80716266-80717459 | 12:81077303-81078935 |
| 12:81759046-81760278 | 12:84910134-84913856 | 12:85272979-85274273 |
| 12:89355594-89356496 | 12:89631020-89632194 | 12:89962469-89963480 |
| 12:93314762-93315907 | 12:93886249-93887382 | 12:94128022-94129009 |
| 12:94138869-94140057 | 12:96489356-96490539 | 12:96522087-96523211 |
| 12:98745040-98746584 | 12:98780906-98781914 | 12:98893440-98894584 |
| 12:100356400- | 12:100716192- | 12:101209083- |
| 100357572 | 100718557 | 101210733 |
| 12:101641904- | 12:101846984- | 12:101924415- |
| 101643184 | 101848042 | 101925492 |
| 12:102950130- | 12:102960683- | 12:103302008- |
| 102951711 | 102962799 | 103303090 |
| 12:103462566- | 12:103984346- | 12:104722767- |
| 103463611 | 103985328 | 104723943 |
| 12:106580455- | 12:106584684- | 12:107414850- |
| 106584454 | 106587139 | 107415885 |
| 12:107416716- | 12:107577994- | 12:107633292- |
| 107417814 | 107579167 | 107634459 |
| 12:107646164- | 12:107843562- | 12:107845118- |
| 107647333 | 107845284 | 107846248 |
| 12:107903024- | 12:109289382- | 12:109359780- |
| 107904416 | 109291108 | 109360853 |
| 12:109600537- | 12:109618626- | 12:109814774- |
| 109601684 | 109619703 | 109815812 |
| 12:109927170- | 12:110065485- | 12:110631854- |
| 109928271 | 110066538 | 110632969 |
| 12:110956279- | 12:111032807- | 12:111102764- |
| 110959145 | 111036572 | 111103874 |
| 12:111226318- | 12:111309954- | 12:111348796- |
| 111227512 | 111311130 | 111349826 |
| 12:112858190- | 12:113094359- | 12:113468864- |
| 112859237 | 113095554 | 113469932 |
| 12:113478509- | 12:113727576- | 12:113807703- |
| 113480752 | 113728553 | 113808859 |
| 12:114394754- | 12:114396835- | 12:114398852- |
| 114395743 | 114397961 | 114401798 |
| 12:114402467- | 12:114407491- | 12:114413910- |
| 114406975 | 114410323 | 114415138 |
| 12:114446778- | 12:114500686- | 12:114695892- |
| 114449354 | 114501922 | 114698995 |
| 12:114735341- | 12:115166896- | 12:116702436- |
| 114736498 | 115168050 | 116703630 |
| 12:116999804- | 12:118052867- | 12:118908490- |
| 117000982 | 118053969 | 118910338 |
| 12:119008523- | 12:119194190- | 12:119235222- |
| 119009673 | 119195299 | 119236155 |
| 12:119691997- | 12:120361321- | 12:120716140- |
| 119692979 | 120362677 | 120717482 |
| 12:120966423- | 12:121180902- | 12:121185557- |
| 120967568 | 121181908 | 121186692 |
| 12:121578283- | 12:121653759- | 12:121854096- |
| 121579743 | 121654894 | 121855809 |
| 12:122643832- | 12:123493625- | 12:123798934- |
| 122645013 | 123494800 | 123800086 |
| 12:123924686- | 12:124666952- | 12:125093214- |
| 123925795 | 124668104 | 125094492 |
| 12:125104441- | 12:125650239- | 12:125773056- |
| 125106196 | 125651413 | 125774095 |
| 12:126480304- | 12:126727283- | 12:127145463- |
| 126481474 | 126728483 | 127146864 |
| 12:128117126- | 12:128265842- | 12:128644794- |
| 128119172 | 128269798 | 128645950 |
| 12:128862513- | 12:129574899- | 12:129902691- |
| 128863726 | 129576077 | 129905141 |
| 12:129926129- | 12:130711322- | 12:130953486- |
| 129927284 | 130712514 | 130954732 |
| 12:131084152- | 12:131123481- | 12:131533312- |
| 131085337 | 131125828 | 131534660 |
| 12:131557686- | 12:131584100- | 12:131616885- |
| 131559427 | 131585286 | 131618243 |
| 12:131667596- | 12:131692104- | 12:131711841- |
| 131668659 | 131693423 | 131712967 |
| 12:131839929- | 12:131843961- | 12:131864847- |
| 131841137 | 131846506 | 131865999 |
| 12:132180293- | 12:132186533- | 12:132192386- |
| 132181468 | 132187936 | 132193551 |
| 12:132200308- | 12:132205389- | 12:132275416- |
| 132201472 | 132206589 | 132276584 |
| 12:132367124- | 12:132369049- | 12:132391999- |
| 132368290 | 132370635 | 132393200 |
| 12:132441686- | 12:132442712- | 12:132461457- |
| 132442870 | 132444752 | 132462673 |
| 12:132617918- | 12:132851138- | 13:20161345-20162536 |
| 132620281 | 132852297 | |
| 13:20968385-20969354 | 13:21723785-21724959 | 13:21817067-21818131 |
| 13:22848098-22849245 | 13:22926018-22927283 | 13:23455924-23456961 |
| 13:23714784-23715849 | 13:25169951-25172439 | 13:25227228-25228392 |
| 13:27274764-27275710 | 13:27791910-27794603 | 13:27794380-27796013 |
| 13:27822451-27823580 | 13:27922674-27925360 | 13:27926193-27930215 |
| 13:27970078-27971690 | 13:27974925-27976302 | 13:27976443-27977588 |
| 13:27980111-27981305 | 13:28014265-28015269 | 13:28129305-28130285 |
| 13:28389053-28390079 | 13:28451119-28452284 | 13:30905910-30907101 |
| 13:31004643-31005816 | 13:31259431-31260462 | 13:32311729-32312631 |
| 13:32876045-32877069 | 13:34749995-34751087 | 13:35470469-35472084 |
| 13:35474629-35477448 | 13:35477906-35479527 | 13:36130339-36132020 |
| 13:36154281-36155580 | 13:36214079-36215239 | 13:36344642-36347247 |
| 13:36369706-36370757 | 13:36370948-36371898 | 13:36429829-36433201 |
| 13:36872322-36873349 | 13:36903918-36904935 | 13:39538660-39539672 |
| 13:40139733-40140866 | 13:40141481-40142604 | 13:40225686-40226837 |
| 13:41106674-41107809 | 13:43194814-43195831 | 13:43360545-43361720 |
| 13:44755464-44756462 | 13:44987573-44988739 | 13:45615184-45616450 |
| 13:45850221-45852342 | 13:46212871-46213936 | 13:46386287-46388386 |
| 13:47903872-47905101 | 13:49219417-49221929 | 13:50131959-50133285 |
| 13:50148618-50149770 | 13:50842759-50844387 | 13:51334944-51336266 |
| 13:51346226-51347222 | 13:52738248-52740542 | 13:52844894-52846890 |
| 13:52847969-52851172 | 13:57628882-57632938 | 13:57633614-57635545 |
| 13:60473297-60474363 | 13:61557716-61558941 | 13:70106693-70107636 |
| 13:72221216-72222413 | 13:73870724-73871731 | 13:77918187-77920326 |
| 13:77994662-77995776 | 13:78029677-78030743 | 13:78053275-78054265 |
| 13:78586246-78588084 | 13:78594990-78597931 | 13:78600622-78604380 |
| 13:78606742-78608639 | 13:78659558-78660669 | 13:83878277-83880725 |
| 13:87080083-87081209 | 13:87676832-87678942 | 13:93915475-93916661 |
| 13:95432536-95433587 | 13:95472989-95474160 | 13:95549673-95550645 |
| 13:95643298-95645403 | 13:96053821-96055006 | 13:97677777-97678828 |
| 13:99314053-99315692 | 13:99870416-99871420 | 13:99894468-99896858 |
| 13:99955267-99956584 | 13:99958119-99959353 | 13:99960411-99961561 |
| 13:99971304-99973310 | 13:99974320-99976302 | 13:99977584-99978717 |
| 13:99988089-99990384 | 13:99990117-99992853 | 13:99993666-99994652 |
| 13:99996360-99998146 | 13:100518934- | 13:100566367- |
| | 100520100 | 100567410 |
| 13:101919388- | 13:105505420- | 13:106472547- |
| 101920535 | 105506326 | 106473660 |
| 13:108494377- | 13:109727528- | 13:109784147- |
| 108497421 | 109728687 | 109785328 |
| 13:109793529- | 13:110134512- | 13:110199666- |
| 109794692 | 110135636 | 110200813 |
| 13:110209266- | 13:110214442- | 13:110228959- |
| 110210406 | 110215582 | 110230002 |
| 13:110244599- | 13:110329264- | 13:110412679- |
| 110245765 | 110330387 | 110413820 |
| 13:110424219- | 13:110634171- | 13:110756498- |
| 110425361 | 110635369 | 110757539 |
| 13:110811797- | 13:110936577- | 13:111120988- |
| 110812988 | 110937771 | 111122274 |
| 13:111324301- | 13:111508362- | 13:111534247- |
| 111327330 | 111509526 | 111535622 |
| 13:111538280- | 13:111627177- | 13:111892466- |
| 111539450 | 111628358 | 111894285 |
| 13:111965413- | 13:112000172- | 13:112054436- |
| 111966577 | 112001292 | 112055740 |
| 13:112055513- | 13:112060556- | 13:112065264- |
| 112059034 | 112063993 | 112068009 |
| 13:112069393- | 13:112103463- | 13:112225015- |
| 112070783 | 112108024 | 112226168 |
| 13:112227946- | 13:112239222- | 13:112338049- |
| 112229109 | 112241817 | 112339679 |
| 13:112614930- | 13:112617123- | 13:112757112- |
| 112616042 | 112618200 | 112758233 |
| 13:112910982- | 13:113109315- | 13:113594051- |
| 112912152 | 113111670 | 113595195 |
| 13:113981238- | 13:114004007- | 13:114090024- |
| 113982416 | 114005800 | 114091182 |
| 14:21286684-21288332 | 14:21995674-21997413 | 14:22084563-22085615 |
| 14:23351428-23353650 | 14:23418710-23419734 | 14:23431278-23432319 |
| 14:23575739-23578441 | 14:23969388-23970562 | 14:24334022-24335725 |
| 14:24670617-24671524 | 14:26204300-26205914 | 14:26401082-26402210 |
| 14:28755120-28756294 | 14:28756527-28757720 | 14:28758136-28760794 |
| 14:28761530-28762698 | 14:28764894-28767929 | 14:28767690-28768873 |
| 14:28772287-28775625 | 14:28783465-28784379 | 14:31014663-31015886 |
| 14:32127979-32129115 | 14:33212334-33213369 | 14:33331380-33332405 |
| 14:33764122-33765171 | 14:36503411-36506654 | 14:36506772-36507697 |
| 14:36508908-36509920 | 14:36509952-36511140 | 14:36512844-36514920 |
| 14:36516588-36522494 | 14:36522197-36523378 | 14:36524028-36526290 |
| 14:36663912-36665041 | 14:36666092-36667283 | 14:38254479-38256334 |
| 14:38522984-38523906 | 14:41604348-41609034 | 14:42807593-42808688 |
| 14:43938544-43939833 | 14:46919577-46920548 | 14:49688383-49689504 |
| 14:49975413-49976450 | 14:50978730-50979918 | 14:50981775-50982928 |
| 14:51093428-51096547 | 14:51351243-51352279 | 14:51635407-51636480 |
| 14:52304001-52304902 | 14:52828362-52829438 | 14:52863978-52865208 |
| 14:52953739-52954777 | 14:53195536-53196644 | 14:53946452-53947716 |
| 14:53952567-53953695 | 14:56271470-56272472 | 14:56585305-56586355 |
| 14:56793598-56795971 | 14:56796780-56799789 | 14:56803746-56804887 |
| 14:56807439-56810125 | 14:56809938-56813496 | 14:57864803-57867033 |
| 14:58132380-58133706 | 14:58662896-58663890 | 14:59629933-59631124 |
| 14:59712940-59713955 | 14:59776581-59777630 | 14:60484788-60486678 |
| 14:60506142-60508312 | 14:60508231-60512134 | 14:60514330-60515913 |
| 14:60637034-60638844 | 14:60641500-60644584 | 14:60645810-60646956 |
| 14:60651465-60653487 | 14:60653409-60654516 | 14:60654738-60656760 |
| 14:61436567-61437629 | 14:68412084-68413083 | 14:70221746-70222686 |
| 14:70233961-70235290 | 14:71309693-71310718 | 14:71620200-71621102 |
| 14:72403587-72404695 | 14:72611340-72612442 | 14:72679916-72681038 |
| 14:72708822-72709966 | 14:72731150-72732207 | 14:73712943-73714108 |
| 14:74238751-74242205 | 14:74470459-74471572 | 14:74999930-75000942 |
| 14:77133309-77134462 | 14:77273014-77274055 | 14:77501527-77502702 |
| 14:78172899-78174460 | 14:78616873-78618377 | 14:78784943-78785936 |
| 14:79278062-79281034 | 14:83225203-83226394 | 14:85528931-85530630 |
| 14:85531555-85532875 | 14:90140122-90141257 | 14:90254642-90255591 |
| 14:90343996-90345035 | 14:90575510-90576703 | 14:90585414-90586561 |
| 14:90599833-90600990 | 14:91832394-91833345 | 14:92202267-92203313 |
| 14:92274193-92275294 | 14:92390181-92391275 | 14:92438737-92439838 |
| 14:92912542-92913444 | 14:92937367-92938503 | 14:93239687-93240840 |
| 14:93436989-93438214 | 14:93718744-93719721 | 14:93719639-93720803 |
| 14:93720862-93722219 | 14:94503787-94504839 | 14:94772079-94774681 |
| 14:95026743-95027854 | 14:95273421-95274510 | 14:95441120-95442706 |
| 14:96591951-96593609 | 14:96623439-96624628 | 14:96930836-96931939 |
| 14:96964345-96965493 | 14:97032550-97034250 | 14:97153068-97154409 |
| 14:97206391-97207989 | 14:97412817-97413892 | 14:97426538-97427613 |
| 14:97788813-97790921 | 14:99659257-99660947 | 14:99717015-99718139 |
| 14:99891612-99892774 | 14:100134873- | 14:100734772- |
| | 100136173 | 100735866 |
| 14:100829842- | 14:100833423- | 14:100879252- |
| 100831088 | 100834515 | 100881596 |
| 14:100882385- | 14:100893254- | 14:100915717- |
| 100883746 | 100894365 | 100916670 |
| 14:100938905- | 14:100980558- | 14:101038342- |
| 100939956 | 100983479 | 101039384 |
| 14:101058996- | 14:101064700- | 14:101125122- |
| 101063301 | 101067106 | 101126313 |
| 14:101247613- | 14:101457660- | 14:101545149- |
| 101249423 | 101458859 | 101546296 |
| 14:101558971- | 14:101562281- | 14:101589669- |
| 101562032 | 101563462 | 101590853 |
| 14:101952721- | 14:102753703- | 14:103308652- |
| 101954110 | 102754816 | 103309783 |
| 14:103549706- | 14:104016716- | 14:104174804- |
| 103550886 | 104017680 | 104176584 |
| 14:104331495- | 14:104636046- | 14:104821955- |
| 104332689 | 104637749 | 104823081 |
| 14:105079776- | 14:105405044- | 14:105631603- |
| 105080954 | 105406119 | 105632707 |
| 14:105986774- | 15:23386500-23387621 | 15:23446771-23447893 |
| 105988187 | | |
| 15:23877511-23878856 | 15:25093414-25094492 | 15:25707551-25709493 |
| 15:25716231-25718034 | 15:25734283-25735433 | 15:26545521-26546586 |
| 15:26771080-26774807 | 15:28967575-28968661 | 15:29103138-29104594 |
| 15:29114487-29116250 | 15:29120769-29121800 | 15:29135942-29137392 |
| 15:29149038-29150219 | 15:29206741-29207907 | 15:29967669-29969752 |
| 15:30882615-30883770 | 15:30921572-30922804 | 15:32716346-32718927 |
| 15:33464549-33466089 | 15:33649435-33650567 | 15:33837393-33838506 |
| 15:34428484-34429621 | 15:34433899-34435038 | 15:34655424-34656442 |
| 15:34751052-34751954 | 15:34753958-34755794 | 15:35271208-35272237 |
| 15:36879759-36880905 | 15:36925314-36926298 | 15:37820459-37821551 |
| 15:38452745-38454185 | 15:39833353-39834350 | 15:40116420-40117538 |
| 15:40282628-40284107 | 15:40428769-40429852 | 15:40951839-40952990 |
| 15:42806789-42807747 | 15:43279197-43280284 | 15:43744708-43745869 |
| 15:45116386-45117709 | 15:45251539-45252665 | 15:45703872-45705073 |
| 15:47716561-47719494 | 15:47772791-47773870 | 15:48190292-48192650 |
| 15:48207529-48208584 | 15:48504498-48505590 | 15:48671784-48672844 |
| 15:49865994-49867148 | 15:52295820-52296812 | 15:52392330-52393462 |
| 15:52782928-52786460 | 15:52786646-52791935 | 15:55868346-55869358 |
| 15:56018982-56020017 | 15:57235538-57236722 | 15:58064398-58067150 |
| 15:58137369-58138271 | 15:59268845-59269921 | 15:59275945-59277087 |
| 15:59438591-59439677 | 15:59523690-59524623 | 15:59993602-59996740 |
| 15:60001363-60006441 | 15:60371685-60372823 | 15:60681097-60682079 |
| 15:61047802-61048919 | 15:61087743-61088699 | 15:61210722-61212406 |
| 15:62847433-62848574 | 15:63025232-63026309 | 15:63320888-63322302 |
| 15:63587748-63588775 | 15:63950871-63951901 | 15:64823457-64824927 |
| 15:64914317-64915993 | 15:65396134-65397557 | 15:65420563-65421716 |
| 15:65902961-65903982 | 15:66462407-66463446 | 15:67033188-67034368 |
| 15:67792293-67793324 | 15:67824657-67830400 | 15:68187021-68188889 |
| 15:68313685-68314846 | 15:68331798-68333071 | 15:68337885-68338935 |
| 15:68350199-68351361 | 15:68569718-68570718 | 15:68596924-68597944 |
| 15:68794854-68796024 | 15:68930886-68932035 | 15:69292802-69293918 |
| 15:69415134-69416492 | 15:70397144-70398263 | 15:72307470-72308418 |
| 15:72397882-72398844 | 15:73366934-73370124 | 15:73682384-73683486 |
| 15:74133982-74135754 | 15:74304742-74305790 | 15:74343353-74344481 |
| 15:74618631-74619669 | 15:74722325-74723372 | 15:76337159-76338919 |
| 15:76386038-76386962 | 15:78675935-78677082 | 15:78830458-78831630 |
| 15:79064129-79065420 | 15:79157423-79158585 | 15:79431095-79433858 |
| 15:80397503-80399170 | 15:80596355-80597868 | 15:80905950-80907093 |
| 15:81195115-81197448 | 15:81612138-81613179 | 15:82093516-82094614 |
| 15:82556063-82557920 | 15:82646912-82649240 | 15:82802558-82803706 |
| 15:83106387-83108614 | 15:83115419-83116499 | 15:83120394-83122169 |
| 15:83282329-83286020 | 15:83378621-83380151 | 15:83489720-83490736 |
| 15:83844449-83845621 | 15:83944357-83945269 | 15:85453805-85454903 |
| 15:88254766-88258768 | 15:88604104-88606377 | 15:88704631-88706261 |
| 15:88708841-88710003 | 15:88710969-88712285 | 15:88912056-88913223 |
| 15:89221006-89222424 | 15:89366918-89368680 | 15:89378273-89379437 |
| 15:89408068-89410632 | 15:89410734-89412012 | 15:89648891-89649982 |
| 15:89690887-89692043 | 15:90823970-90825098 | 15:90867074-90868192 |
| 15:91024369-91025516 | 15:91693725-91694670 | 15:91704832-91705734 |
| 15:92580103-92581697 | 15:92746940-92747870 | 15:93046531-93047562 |
| 15:93105642-93107066 | 15:93121785-93122939 | 15:96358994-96360171 |
| 15:96360362-96362310 | 15:96362563-96364223 | 15:98420948-98422149 |
| 15:98423804-98425393 | 15:99005627-99006642 | 15:99124742-99125931 |
| 15:100878355- | 15:100879830- | 15:101103951- |
| 100879542 | 100881378 | 101105051 |
| 15:101152244- | 15:101663935- | 16:53945-55260 |
| 101153288 | 101665104 | |
| 16:164867-167209 | 16:420571-421671 | 16:539492-540681 |
| 16:810132-811310 | 16:833159-834384 | 16:1151874-1154580 |
| 16:1171358-1173212 | 16:1397413-1398581 | 16:1487180-1488928 |
| 16:1717099-1718244 | 16:1990909-1992700 | 16:2142784-2143932 |
| 16:2331953-2333125 | 16:2341231-2342426 | 16:2680521-2681589 |
| 16:2751370-2752500 | 16:3091635-3093603 | 16:3183748-3184889 |
| 16:3206758-3208431 | 16:3273690-3274713 | 16:3365511-3366665 |
| 16:3560067-3561179 | 16:3562917-3565493 | 16:3938097-3939675 |
| 16:3962393-3963321 | 16:3966118-3967295 | 16:3988618-3989770 |
| 16:4207553-4208718 | 16:4537167-4539276 | 16:4904400-4905588 |
| 16:4991300-4992480 | 16:6018283-6020884 | 16:6482568-6484388 |
| 16:8736492-8737655 | 16:9012762-9013940 | 16:11844338-11845456 |
| 16:11967256-11968395 | 16:12297014-12298213 | 16:12900545-12904385 |
| 16:14973066-14974104 | 16:15501546-15502958 | 16:15532461-15533586 |
| 16:15784158-15785347 | 16:17941679-17942784 | 16:18790652-18791795 |
| 16:19094459-19095625 | 16:19661851-19663023 | 16:20690971-20692001 |
| 16:20964139-20965303 | 16:21166975-21167989 | 16:21707877-21709056 |
| 16:22026057-22027101 | 16:22812959-22815373 | 16:23713721-23714819 |
| 16:24234384-24236004 | 16:24670513-24671612 | 16:24870422-24871435 |
| 16:27180864-27181816 | 16:27717738-27718964 | 16:28062461-28064550 |
| 16:28982500-28983664 | 16:29194171-29195180 | 16:29216824-29217861 |
| 16:29777187-29778381 | 16:30094815-30096120 | 16:30611169-30612219 |
| 16:30820447-30821992 | 16:31013967-31015020 | 16:31215578-31217506 |
| 16:31370931-31372072 | 16:31427183-31428461 | 16:31536811-31538186 |
| 16:31546096-31548088 | 16:46573218-46574519 | 16:46730458-46732024 |
| 16:48496174-48497348 | 16:49273971-49276110 | 16:49279746-49284455 |
| 16:49414287-49415439 | 16:49454520-49455993 | 16:49483642-49485106 |
| 16:49578299-49579424 | 16:49637500-49639373 | 16:49709369-49710412 |
| 16:49748802-49749862 | 16:49789964-49791094 | 16:50519070-50520134 |
| 16:50548777-50549957 | 16:50840633-50841964 | 16:50943450-50944566 |
| 16:51113053-51114551 | 16:51133342-51135224 | 16:51149484-51152955 |
| 16:51152933-51154431 | 16:54060343-54061245 | 16:54594251-54595475 |
| 16:55444902-55446620 | 16:55655053-55657539 | 16:56121277-56122329 |
| 16:56163659-56164583 | 16:56190017-56193732 | 16:56193622-56195187 |
| 16:56328435-56329593 | 16:56662579-56663768 | 16:56885038-56886122 |
| 16:56893914-56895058 | 16:57769738-57770880 | 16:57911241-57912360 |
| 16:57971061-57972081 | 16:58467201-58468307 | 16:58500913-58502103 |
| 16:58664416-58665556 | 16:62034053-62037553 | 16:65705070-65706156 |
| 16:66385121-66386240 | 16:67004867-67005920 | 16:68893263-68894409 |
| 16:69083183-69084299 | 16:69830846-69831929 | 16:71536448-71537694 |
| 16:72946737-72947888 | 16:74076763-74077755 | 16:75563481-75564510 |
| 16:77589696-77591272 | 16:77975007-77976190 | 16:78761009-78762108 |
| 16:79201583-79202605 | 16:79347148-79348343 | 16:79826699-79828236 |
| 16:79980681-79981681 | 16:80632711-80633758 | 16:80675348-80676501 |
| 16:80734987-80735937 | 16:80892187-80893352 | 16:81071317-81072297 |
| 16:81890947-81892121 | 16:81913778-81914907 | 16:81930786-81931832 |
| 16:82172321-82173223 | 16:83625686-83626867 | 16:83752508-83753675 |
| 16:84225209-84226281 | 16:84260622-84261796 | 16:84851106-84852130 |
| 16:84940233-84941421 | 16:85098253-85100177 | 16:85107162-85108772 |
| 16:85221888-85223211 | 16:85644188-85645326 | 16:85902352-85903444 |
| 16:86494139-86496951 | 16:86497505-86498694 | 16:86498830-86499977 |
| 16:86507287-86509414 | 16:86512423-86513616 | 16:86513322-86514444 |
| 16:86514692-86516262 | 16:86516254-86517437 | 16:86565551-86569332 |
| 16:86579537-86580576 | 16:86732507-86733807 | 16:86878594-86879722 |
| 16:86916419-86918386 | 16:87172128-87173244 | 16:87192003-87193039 |
| 16:87194559-87195698 | 16:87200695-87201799 | 16:87388944-87390060 |
| 16:87598190-87599193 | 16:87642056-87643367 | 16:87688034-87689213 |
| 16:87761612-87763268 | 16:88024045-88025212 | 16:88130209-88131560 |
| 16:88132697-88133874 | 16:88566189-88567243 | 16:88876206-88878053 |
| 16:88930445-88931554 | 16:88981423-88982624 | 16:88999827-89001139 |
| 16:89019905-89021040 | 16:89084457-89086486 | 16:89152818-89153999 |
| 16:89158135-89159216 | 16:89201953-89203145 | 16:89970736-89972650 |
| 17:439783-440946 | 17:441201-442310 | 17:442625-444289 |
| 17:820459-821621 | 17:835012-836170 | 17:932213-933255 |
| 17:1255587-1256616 | 17:1939223-1940756 | 17:1944295-1945477 |
| 17:1977443-1978598 | 17:2057221-2058499 | 17:3419973-3421065 |
| 17:3759460-3760453 | 17:5115785-5116968 | 17:5498617-5499698 |
| 17:5499790-5501764 | 17:5758007-5759059 | 17:5793743-5794645 |
| 17:6712517-6714466 | 17:6749410-6750312 | 17:7074017-7075176 |
| 17:7087480-7088594 | 17:7410364-7411497 | 17:7418877-7419847 |
| 17:7588883-7590077 | 17:7707100-7708557 | 17:7769491-7770574 |
| 17:8072010-8073790 | 17:8332199-8333244 | 17:8504002-8505165 |
| 17:8555738-8556850 | 17:8621325-8622298 | 17:8821727-8822641 |
| 17:8880140-8881177 | 17:8922619-8923670 | 17:8945561-8947160 |
| 17:9021838-9024053 | 17:9128066-9129219 | 17:9239919-9241069 |
| 17:9402327-9403441 | 17:9970343-9971460 | 17:9984321-9985439 |
| 17:10247774-10248871 | 17:10372660-10373656 | 17:10382465-10383641 |
| 17:10518860-10519887 | 17:10655450-10656661 | 17:10657903-10659038 |
| 17:14345056-14346125 | 17:14987623-14988993 | 17:15916717-15918267 |
| 17:16619188-16620571 | 17:16666579-16667754 | 17:16976759-16978607 |
| 17:17314769-17315729 | 17:21062028-21063051 | 17:27082071-27084133 |
| 17:27418996-27420068 | 17:27551653-27552805 | 17:27670315-27671389 |
| 17:28371554-28373033 | 17:29004715-29006948 | 17:29241861-29242763 |
| 17:30345138-30346180 | 17:30359598-30360722 | 17:30377330-30378638 |
| 17:30921885-30923600 | 17:31390629-31392886 | 17:31727748-31728694 |
| 17:31754132-31755068 | 17:33500740-33501731 | 17:33753089-33754177 |
| 17:34119680-34121568 | 17:34625730-34627147 | 17:34825829-34827014 |
| 17:35144509-35145452 | 17:35448737-35450248 | 17:35489612-35490711 |
| 17:35495408-35497229 | 17:35497777-35498936 | 17:35939961-35940937 |
| 17:36657379-36658696 | 17:36721786-36722948 | 17:36885203-36886313 |
| 17:36933270-36935218 | 17:36938598-36942565 | 17:36945449-36946741 |
| 17:37509693-37511378 | 17:38578067-38579518 | 17:39064644-39065666 |
| 17:40288392-40289518 | 17:40753373-40754411 | 17:40777632-40778634 |
| 17:41350261-41351468 | 17:41423882-41424890 | 17:41441110-41442179 |
| 17:42165872-42166990 | 17:42311737-42313199 | 17:42653248-42654416 |
| 17:43754816-43756206 | 17:43841427-43842835 | 17:43974517-43975620 |
| 17:44264600-44265783 | 17:44655703-44657775 | 17:44664274-44665306 |
| 17:44969586-44971054 | 17:45893187-45896655 | 17:45896393-45898246 |
| 17:46850393-46852774 | 17:46911347-46912512 | 17:47871926-47873106 |
| 17:48591622-48594333 | 17:48594962-48597629 | 17:48627973-48629166 |
| 17:48641312-48643288 | 17:48704107-48705125 | 17:48721689-48723834 |
| 17:48732372-48734734 | 17:48738093-48739267 | 17:48746427-48748589 |
| 17:48749154-48750831 | 17:48753399-48754831 | 17:48754677-48755868 |
| 17:49035097-49036864 | 17:49223414-49225241 | 17:49992752-49995075 |
| 17:50200966-50202200 | 17:50214772-50215821 | 17:50568339-50569421 |
| 17:50699000-50700013 | 17:51699004-51700147 | 17:52157365-52160964 |
| 17:53821947-53823762 | 17:55457984-55459163 | 17:55561819-55562989 |
| 17:55873445-55874551 | 17:56636821-56637957 | 17:56724275-56725358 |
| 17:57214938-57215871 | 17:58520963-58522093 | 17:58543530-58545126 |
| 17:60139026-60140595 | 17:60908610-60909609 | 17:61395713-61398161 |
| 17:61398396-61401678 | 17:61403998-61405685 | 17:61451357-61453341 |
| 17:61456721-61458343 | 17:62643994-62645367 | 17:62763828-62764966 |
| 17:63535395-63536568 | 17:63537950-63539124 | 17:63956390-63957422 |
| 17:64464825-64465935 | 17:64732353-64733491 | 17:64777880-64780193 |
| 17:66348655-66349923 | 17:66828992-66830577 | 17:66834352-66835475 |
| 17:67061089-67062118 | 17:67266106-67267128 | 17:68162343-68163275 |
| 17:68328819-68329922 | 17:69042163-69043124 | 17:72116782-72119080 |
| 17:72658457-72659566 | 17:73497353-73498503 | 17:73881679-73882706 |
| 17:73951723-73953066 | 17:74247191-74250495 | 17:74261530-74262642 |
| 17:74347716-74348900 | 17:74351420-74352621 | 17:74352966-74354054 |
| 17:74530913-74532643 | 17:74614505-74615557 | 17:74670971-74672151 |
| 17:74842748-74843942 | 17:76076274-76077938 | 17:76303796-76304896 |
| 17:76673279-76674374 | 17:77031036-77032043 | 17:77233060-77234205 |
| 17:77557589-77559666 | 17:77692976-77694114 | 17:77870893-77872019 |
| 17:77879115-77880248 | 17:78104467-78105531 | 17:78117068-78118160 |
| 17:78468117-78469301 | 17:78491996-78493169 | 17:78861566-78862764 |
| 17:79144986-79146873 | 17:79218473-79219525 | 17:79494309-79495400 |
| 17:79801437-79802628 | 17:79851462-79852525 | 17:79999752-80000912 |
| 17:80111268-80113227 | 17:80945381-80946559 | 17:81298078-81299229 |
| 17:81340867-81343641 | 17:81380354-81381980 | 17:81389843-81391571 |
| 17:81398578-81401316 | 17:81463183-81464902 | 17:82065848-82067035 |
| 17:82374788-82375981 | 17:82586837-82588034 | 17:82921028-82922198 |
| 17:82966491-82967817 | 17:83028980-83030148 | 18:489771-490816 |
| 18:903374-910087 | 18:1806842-1807984 | 18:2893607-2894767 |
| 18:3050847-3051934 | 18:3601489-3602564 | 18:4453304-4456444 |
| 18:5196140-5198153 | 18:5894070-5896868 | 18:6241084-6243022 |
| 18:6434344-6435393 | 18:6482336-6483381 | 18:6981993-6983064 |
| 18:7467277-7468351 | 18:10316497-10317627 | 18:10469953-10471010 |
| 18:10473984-10475102 | 18:10603884-10604952 | 18:10685593-10686734 |
| 18:10696538-10697485 | 18:10729669-10730571 | 18:11111005-11112020 |
| 18:11147203-11148356 | 18:11489686-11490588 | 18:11660540-11661645 |
| 18:11669662-11671615 | 18:12061256-12062388 | 18:12063835-12064964 |
| 18:12253238-12255540 | 18:12399621-12400667 | 18:13193672-13194697 |
| 18:13202353-13203535 | 18:13294906-13295934 | 18:13480440-13481342 |
| 18:14377896-14378952 | 18:14747439-14748891 | 18:15197273-15198760 |
| 18:21240765-21243999 | 18:22065779-22066781 | 18:23237823-23238825 |
| 18:24268861-24269902 | 18:25223900-25225397 | 18:25989670-25990736 |
| 18:26469868-26471059 | 18:26547311-26548503 | 18:26550570-26551984 |
| 18:31040365-31042815 | 18:31963057-31964241 | 18:33439702-33441552 |
| 18:35266870-35268616 | 18:36760860-36761840 | 18:37253377-37255234 |
| 18:37684072-37684984 | 18:44926689-44927653 | 18:45063163-45064370 |
| 18:45064623-45065684 | 18:45315836-45316831 | 18:45774875-45776040 |
| 18:46556801-46557888 | 18:46558683-46560025 | 18:46646224-46647259 |
| 18:46947439-46948600 | 18:47249064-47250056 | 18:47262980-47265017 |
| 18:48714454-48715525 | 18:48942075-48943238 | 18:50097049-50098251 |
| 18:51150015-51151033 | 18:55347213-55348179 | 18:55433028-55433943 |
| 18:55544404-55545428 | 18:56433469-56434522 | 18:57351842-57353687 |
| 18:57427396-57429254 | 18:57439282-57442214 | 18:57706381-57707398 |
| 18:59272584-59275073 | 18:59300017-59301026 | 18:60010569-60011684 |
| 18:62199170-62200233 | 18:64018999-64020069 | 18:64331521-64332559 |
| 18:65749286-65752336 | 18:68842269-68843443 | 18:69402157-69403611 |
| 18:72866280-72869290 | 18:74314871-74316046 | 18:74497672-74498756 |
| 18:75204544-75205746 | 18:75320974-75322138 | 18:75427819-75429537 |
| 18:75454139-75456531 | 18:75489509-75490560 | 18:76322155-76323298 |
| 18:77248589-77251423 | 18:77261979-77262988 | 18:77676475-77677591 |
| 18:77749184-77750364 | 18:77863182-77864209 | 18:78543780-78545255 |
| 18:78584544-78585595 | 18:78724997-78726148 | 18:78976205-78980690 |
| 18:78994300-78995476 | 18:79003703-79004834 | 18:79021200-79022918 |
| 18:79053868-79054843 | 18:79572814-79573824 | 18:79806649-79808150 |
| 18:79848138-79849399 | 18:80108312-80109214 | 18:80245764-80246953 |
| 19:403279-404459 | 19:443934-445106 | 19:684722-685830 |
| 19:687628-688716 | 19:939015-940361 | 19:1050527-1051675 |
| 19:1079107-1080622 | 19:1466408-1469065 | 19:1523319-1524637 |
| 19:1629676-1630809 | 19:1806388-1807580 | 19:1969387-1971568 |
| 19:2250300-2254444 | 19:2286228-2287381 | 19:2289446-2291859 |
| 19:2650368-2651672 | 19:2691556-2692677 | 19:3771083-3772254 |
| 19:4174771-4175961 | 19:4206859-4208268 | 19:4906452-4907492 |
| 19:5222424-5223591 | 19:5230837-5232028 | 19:5236635-5237819 |
| 19:5247156-5248303 | 19:5333618-5335700 | 19:6427037-6428227 |
| 19:6433236-6434425 | 19:6685564-6686598 | 19:6688366-6689392 |
| 19:7861721-7863682 | 19:8536516-8537756 | 19:8587881-8588967 |
| 19:8589031-8590226 | 19:8598685-8599844 | 19:8602498-8603688 |
| 19:8609026-8610206 | 19:8697878-8699071 | 19:9497678-9499117 |
| 19:9965862-9967053 | 19:10030810-10032004 | 19:10295667-10296881 |
| 19:10838062-10839117 | 19:11354600-11355502 | 19:11479781-11482738 |
| 19:12076674-12077710 | 19:12194143-12195939 | 19:12513282-12514469 |
| 19:12834132-12835293 | 19:12840586-12842065 | 19:13013756-13014938 |
| 19:13235452-13237059 | 19:13333613-13334624 | 19:13977873-13979490 |
| 19:14940619-14942611 | 19:15161065-15162194 | 19:15231341-15233561 |
| 19:15469004-15470502 | 19:15519402-15520475 | 19:15550716-15552255 |
| 19:17025818-17027816 | 19:17293784-17294975 | 19:17613945-17615612 |
| 19:17981171-17982572 | 19:18009211-18010402 | 19:18081757-18082879 |
| 19:18895906-18897108 | 19:19232104-19233311 | 19:19258762-19259917 |
| 19:19260518-19262140 | 19:19262242-19263397 | 19:19515019-19516218 |
| 19:19860189-19861567 | 19:20566332-20567455 | 19:21395136-21396193 |
| 19:21585828-21587526 | 19:22051740-22053648 | 19:22632449-22635570 |
| 19:23060959-23061967 | 19:23070745-23072139 | 19:23203397-23204433 |
| 19:23470899-23472062 | 19:23762388-23763701 | 19:27777322-27778451 |
| 19:28502882-28504049 | 19:28933164-28934176 | 19:29330050-29331096 |
| 19:29526012-29529855 | 19:29529783-29530910 | 19:29763997-29765116 |
| 19:30124415-30125410 | 19:31139758-31141333 | 19:31210052-31211169 |
| 19:31352671-31353851 | 19:31356483-31357971 | 19:31587494-31588605 |
| 19:32041033-32042664 | 19:32861830-32863027 | 19:33100654-33101675 |
| 19:33210524-33211656 | 19:33224956-33226193 | 19:33533442-33534627 |
| 19:33537519-33538572 | 19:33683610-33685174 | 19:33791897-33793756 |
| 19:34743074-34744157 | 19:34904445-34906138 | 19:35039880-35041069 |
| 19:35105809-35106994 | 19:35557620-35559130 | 19:35797349-35798495 |
| 19:35843642-35845014 | 19:35860281-35861472 | 19:35958618-35960571 |
| 19:35978718-35979872 | 19:36244982-36246256 | 19:36328561-36330026 |
| 19:36640159-36641202 | 19:36858293-36859373 | 19:37251241-37252423 |
| 19:37462793-37463827 | 19:37507841-37508830 | 19:37828781-37829795 |
| 19:37854781-37855970 | 19:37940701-37941678 | 19:38255934-38257337 |
| 19:38394001-38396696 | 19:38506112-38507311 | 19:38564473-38566173 |
| 19:38592849-38593982 | 19:38613339-38614449 | 19:38944631-38945744 |
| 19:38949284-38950466 | 19:39253800-39254932 | 19:39263750-39265526 |
| 19:39506776-39508183 | 19:40388958-40390062 | 19:40592078-40593069 |
| 19:41681880-41683044 | 19:41844132-41845335 | 19:41994866-41996037 |
| 19:41998453-41999655 | 19:42153018-42154100 | 19:42595238-42596244 |
| 19:43699004-43700324 | 19:43769208-43770356 | 19:43820115-43821438 |
| 19:43957820-43958844 | 19:44050656-44051829 | 19:44111860-44113030 |
| 19:45385308-45386782 | 19:45952465-45953651 | 19:46412293-46414404 |
| 19:46492855-46494211 | 19:46830214-46832014 | 19:47338951-47341363 |
| 19:47446685-47447831 | 19:47456951-47458122 | 19:47544731-47545908 |
| 19:47794066-47795132 | 19:48105971-48107224 | 19:48444158-48445278 |
| 19:48479740-48481644 | 19:49068587-49069637 | 19:49071616-49073543 |
| 19:49132735-49133923 | 19:49324496-49325998 | 19:49421876-49424194 |
| 19:49433078-49434417 | 19:49436217-49437405 | 19:49892151-49893204 |
| 19:50332559-50333708 | 19:50427573-50428954 | 19:50465702-50466877 |
| 19:50548099-50549284 | 19:50638761-50640653 | 19:50683153-50684298 |
| 19:50684346-50685286 | 19:50785852-50788005 | 19:51180882-51182064 |
| 19:51326471-51328301 | 19:51702591-51705061 | 19:51887003-51888678 |
| 19:52344229-52345951 | 19:52397178-52398933 | 19:52492766-52493942 |
| 19:52510446-52511387 | 19:52735355-52736465 | 19:53141286-53142377 |
| 19:53142094-53143398 | 19:53165659-53166773 | 19:53290648-53291816 |
| 19:53308122-53309229 | 19:53511419-53512434 | 19:53522362-53523282 |
| 19:53667419-53668576 | 19:53675009-53675999 | 19:53898118-53899313 |
| 19:53915225-53916343 | 19:53941621-53943181 | 19:53978014-53979151 |
| 19:54036088-54037203 | 19:54163911-54165107 | 19:54413933-54416229 |
| 19:54416133-54417322 | 19:55156642-55157752 | 19:56208128-56209300 |
| 19:56211492-56212676 | 19:56342973-56344125 | 19:56473479-56474499 |
| 19:56476845-56479001 | 19:56625164-56626287 | 19:56637544-56638818 |
| 19:56670850-56672574 | 19:57174784-57175887 | 19:57745752-57746834 |
| 19:57888359-57889545 | 19:58037764-58038935 | 19:58051898-58053063 |
| 19:58084958-58086156 | 19:58100574-58101535 | 19:58330246-58331334 |
| 19:58346868-58348039 | 19:58396456-58397600 | 2:139907-141006 |
| 2:566834-567937 | 2:627691-628719 | 2:692961-693980 |
| 2:949072-952414 | 2:1154140-1155148 | 2:1261394-1262444 |
| 2:1503331-1505249 | 2:1573983-1575040 | 2:1591446-1592740 |
| 2:1632143-1633259 | 2:1700721-1701907 | 2:1817076-1818320 |
| 2:1902594-1903676 | 2:1928825-1929968 | 2:1936747-1938353 |
| 2:2291312-2292512 | 2:2328585-2329499 | 2:2585940-2587120 |
| 2:2688491-2689665 | 2:2793482-2795333 | 2:3105247-3106567 |
| 2:3153805-3154853 | 2:3276543-3278159 | 2:3486978-3488174 |
| 2:3492462-3493922 | 2:3645089-3646129 | 2:3694430-3695349 |
| 2:3701813-3702989 | 2:3702770-3704154 | 2:3766468-3767530 |
| 2:3785670-3786826 | 2:5672778-5674332 | 2:5690413-5692222 |
| 2:6824184-6825284 | 2:6932513-6933534 | 2:7732130-7733259 |
| 2:9475292-9476469 | 2:9665566-9666715 | 2:9685776-9686887 |
| 2:9819867-9821235 | 2:10204267-10205458 | 2:10478656-10479717 |
| 2:10546886-10548314 | 2:10961501-10962923 | 2:11111561-11112719 |
| 2:11471083-11472221 | 2:11561203-11562358 | 2:11591126-11592270 |
| 2:11633557-11635538 | 2:11896327-11897229 | 2:11969013-11970066 |
| 2:15562914-15563927 | 2:15689446-15690554 | 2:15693439-15694449 |
| 2:16560989-16562006 | 2:16726363-16727287 | 2:19356486-19357681 |
| 2:20532216-20533457 | 2:21043029-21044199 | 2:23630521-23631701 |
| 2:23662900-23664071 | 2:25121051-25122215 | 2:25160391-25161580 |
| 2:25170644-25172035 | 2:25328112-25329242 | 2:25349243-25350321 |
| 2:26185540-26186706 | 2:26516237-26517393 | 2:26820507-26821632 |
| 2:26949286-26950466 | 2:27029059-27030097 | 2:27274436-27275443 |
| 2:27306900-27308392 | 2:27573696-27574799 | 2:28809882-28811619 |
| 2:29211647-29212816 | 2:29213474-29214629 | 2:29329886-29331066 |
| 2:29579110-29580182 | 2:30156227-30157213 | 2:30185558-30186589 |
| 2:30820701-30821775 | 2:30957981-30959153 | 2:31068389-31069358 |
| 2:31579546-31581784 | 2:33726852-33729824 | 2:36560327-36561334 |
| 2:36818995-36820010 | 2:37394852-37395896 | 2:37618046-37619054 |
| 2:37949075-37950082 | 2:38072992-38076579 | 2:39392960-39394003 |
| 2:39504860-39505841 | 2:39664760-39667339 | 2:40240815-40241916 |
| 2:40353341-40354389 | 2:43180604-43181655 | 2:44801556-44803173 |
| 2:44927551-44930691 | 2:44932356-44936635 | 2:44940543-44941704 |
| 2:44943849-44945611 | 2:44953946-44955671 | 2:46803899-46805032 |
| 2:47353837-47354893 | 2:47521103-47522297 | 2:47530095-47531201 |
| 2:47664291-47665336 | 2:51026303-51027626 | 2:53569677-53570723 |
| 2:54254746-54256303 | 2:54457279-54458456 | 2:54727986-54729001 |
| 2:55988734-55989636 | 2:56192434-56193635 | 2:58925090-58926194 |
| 2:62724843-62725898 | 2:63047305-63049674 | 2:63055146-63056460 |
| 2:63056230-63060084 | 2:63619094-63620116 | 2:63622049-63623225 |
| 2:64202438-64203457 | 2:64933725-64934760 | 2:64955227-64956377 |
| 2:65474399-65475446 | 2:66431880-66433039 | 2:68654522-68655717 |
| 2:68870621-68871888 | 2:68872419-68873575 | 2:69146658-69147711 |
| 2:69182422-69183414 | 2:69482320-69483321 | 2:70670774-70671911 |
| 2:70676080-70677264 | 2:70751227-70752139 | 2:70887080-70889851 |
| 2:70896745-70897940 | 2:70906818-70908257 | 2:70964421-70965583 |
| 2:71491457-71492508 | 2:71548100-71549905 | 2:71595146-71596329 |
| 2:72022373-72023503 | 2:72146560-72148601 | 2:72919330-72921522 |
| 2:72923480-72926399 | 2:74147777-74148962 | 2:74414744-74415919 |
| 2:74498753-74500185 | 2:74514131-74516896 | 2:80302529-80305325 |
| 2:80321867-80323272 | 2:85258110-85259474 | 2:85583718-85584909 |
| 2:86788404-86791550 | 2:86808966-86810515 | 2:91442144-91443330 |
| 2:94997896-94999067 | 2:95324878-95326041 | 2:96564053-96565192 |
| 2:96985216-96986325 | 2:98345836-98348558 | 2:98396068-98397483 |
| 2:98931634-98932706 | 2:99643532-99644529 | 2:99738718-99739781 |
| 2:99805890-99806965 | 2:99821189-99822285 | 2:99925760-99926842 |
| 2:99933680-99934582 | 2:100012272-100013286 | 2:100142897-100144478 |
| 2:100240595-100242399 | 2:100999196-101000311 | 2:101341975-101343258 |
| 2:102045467-102046565 | 2:104407301-104408247 | 2:104530837-104531975 |
| 2:104569108-104570213 | 2:104794214-104795116 | 2:104842064-104845932 |
| 2:104851943-104857480 | 2:104863498-104864862 | 2:105240901-105243800 |
| 2:105399158-105400378 | 2:107528070-107529280 | 2:108712265-108713417 |
| 2:109174595-109175497 | 2:109300519-109301785 | 2:109457613-109458550 |
| 2:110827975-110829111 | 2:110950675-110951600 | 2:111076319-111077366 |
| 2:111219095-111220143 | 2:112020940-112021984 | 2:112150361-112151694 |
| 2:112181250-112182569 | 2:114661401-114662861 | 2:117835751-117836937 |
| 2:118807963-118809295 | 2:118832977-118834149 | 2:118841410-118843922 |
| 2:118844453-118850887 | 2:118853214-118854849 | 2:118857862-118860112 |
| 2:119156375-119157760 | 2:120216047-120217310 | 2:120653978-120655136 |
| 2:121080188-121081294 | 2:121724065-121725136 | 2:122660598-122662030 |
| 2:123682141-123683697 | 2:124024000-124026265 | 2:126686214-126687304 |
| 2:126695755-126696806 | 2:126702479-126704112 | 2:126885805-126887050 |
| 2:127842359-127843548 | 2:128419762-128420873 | 2:128425334-128426468 |
| 2:128499224-128500262 | 2:128543456-128544450 | 2:128556466-128557571 |
| 2:128635208-128636251 | 2:130273502-130274403 | 2:130755042-130757141 |
| 2:130836932-130838324 | 2:131217684-131218771 | 2:132256468-132257662 |
| 2:134153225-134154389 | 2:135809042-135810262 | 2:135810825-135812058 |
| 2:135815914-135816997 | 2:136304882-136305993 | 2:143334429-143335476 |
| 2:143599565-143600582 | 2:143741865-143742913 | 2:144466258-144467446 |
| 2:144524895-144526007 | 2:144552933-144553929 | 2:145171330-145172360 |
| 2:145588328-145589305 | 2:145739104-145740039 | 2:145776830-145777864 |
| 2:148557527-148558482 | 2:149002347-149003506 | 2:152199782-152200791 |
| 2:152369148-152370055 | 2:153870830-153874160 | 2:160994367-160995440 |
| 2:161416081-161418433 | 2:161426161-161428715 | 2:161625071-161626232 |
| 2:162126382-162127485 | 2:164109762-164110767 | 2:167186621-167187604 |
| 2:168900938-168901840 | 2:169727963-169728988 | 2:170821127-170823676 |
| 2:172080865-172082544 | 2:172087107-172089001 | 2:172092263-172094202 |
| 2:172107572-172108660 | 2:174325552-174329403 | 2:174609282-174610380 |
| 2:175233482-175234422 | 2:176070776-176072755 | 2:176078477-176081771 |
| 2:176082455-176086591 | 2:176095009-176096080 | 2:176103591-176109916 |
| 2:176111472-176113645 | 2:176115479-176118214 | 2:176120089-176124728 |
| 2:176127772-176131377 | 2:176132403-176133577 | 2:176146808-176148530 |
| 2:176150813-176153293 | 2:176156423-176158128 | 2:176157858-176159031 |
| 2:176158807-176160138 | 2:176159868-176164527 | 2:176164483-176166098 |
| 2:176171506-176173191 | 2:176187162-176190444 | 2:176268427-176269455 |
| 2:177092999-177094051 | 2:178416569-178417691 | 2:178563075-178564197 |
| 2:178804149-178805186 | 2:179746748-179747805 | 2:180003633-180004601 |
| 2:181223272-181224174 | 2:181456479-181458752 | 2:181679068-181680235 |
| 2:185738072-185739422 | 2:186848234-186850003 | 2:196809679-196810602 |
| 2:197122303-197123335 | 2:197785575-197787466 | 2:198083345-198084623 |
| 2:199461037-199463210 | 2:199463536-199465669 | 2:199465608-199466636 |
| 2:199466700-199467890 | 2:199468133-199471742 | 2:202034739-202035939 |
| 2:203237326-203238966 | 2:203774322-203775409 | 2:203935681-203936618 |
| 2:205685897-205687042 | 2:206641650-206643383 | 2:207237434-207239123 |
| 2:207249145-207250270 | 2:207680769-207681964 | 2:208164383-208165509 |
| 2:211329539-211330643 | 2:216018521-216019423 | 2:216577264-216578197 |
| 2:216603056-216604084 | 2:216789363-216790469 | 2:216827495-216828547 |
| 2:216868371-216869400 | 2:217223285-217224305 | 2:217475344-217476301 |
| 2:217777233-217778277 | 2:218013791-218014938 | 2:218390705-218392019 |
| 2:218958984-218961178 | 2:219332148-219333542 | 2:219417653-219419320 |
| 2:219483797-219485572 | 2:219496155-219497794 | 2:219522666-219523825 |
| 2:219551114-219553746 | 2:219566226-219568331 | 2:221569928-221571589 |
| 2:222288937-222290060 | 2:222297496-222300699 | 2:222300506-222303398 |
| 2:222303146-222305451 | 2:222307770-222308891 | 2:222311302-222313396 |
| 2:224804972-224806113 | 2:224882968-224883874 | 2:224951590-224952706 |
| 2:224997355-224998541 | 2:226797834-226799029 | 2:227164122-227165525 |
| 2:228179702-228182615 | 2:229972654-229973771 | 2:230705332-230706377 |
| 2:230973851-230974896 | 2:231899895-231901644 | 2:231958176-231959078 |
| 2:232350630-232352102 | 2:232354663-232355591 | 2:232418804-232420532 |
| 2:232485054-232486244 | 2:232486214-232488652 | 2:232661839-232662936 |
| 2:233031845-233033000 | 2:233318766-233319812 | 2:233938329-233939615 |
| 2:234409228-234410311 | 2:235613669-235614806 | 2:235764938-235766103 |
| 2:236154038-236155003 | 2:236159436-236160629 | 2:236163157-236165507 |
| 2:236166890-236170633 | 2:236389135-236390392 | 2:236454832-236456457 |
| 2:236514400-236515520 | 2:236544071-236545221 | 2:236562307-236563357 |
| 2:236567552-236569130 | 2:236585621-236586705 | 2:236605047-236605952 |
| 2:236707966-236709144 | 2:237177820-237179258 | 2:237279550-237280757 |
| 2:237357538-237358559 | 2:237365252-237366412 | 2:237397454-237399058 |
| 2:237465169-237466254 | 2:237626367-237628773 | 2:237690821-237692018 |
| 2:237768903-237770039 | 2:237958037-237959222 | 2:238150418-238151608 |
| 2:238220124-238221205 | 2:238231077-238232310 | 2:238910074-238911185 |
| 2:238913845-238915015 | 2:239048960-239050111 | 2:239076082-239077155 |
| 2:239506725-239508368 | 2:239513768-239514825 | 2:239937856-239938996 |
| 2:240042346-240043483 | 2:240095600-240096761 | 2:240217015-240219245 |
| 2:240792573-240793641 | 2:240813256-240814752 | 2:240935817-240936897 |
| 2:240948275-240949204 | 2:241070737-241072269 | 2:241410198-241412579 |
| 2:241584057-241585269 | 2:241696908-241697989 | 2:241795118-241796278 |
| 2:241803197-241805130 | 2:241824878-241825975 | 2:241945212-241947319 |
| 2:241995799-241997209 | 2:242004179-242005733 | 2:242047534-242048745 |
| 20:642120-643363 | 20:708031-709107 | 20:1045239-1046263 |
| 20:1630428-1631536 | 20:1779277-1780328 | 20:1925983-1927024 |
| 20:1946490-1947754 | 20:2327476-2328655 | 20:2558307-2559498 |
| 20:2793568-2794678 | 20:2820463-2821939 | 20:2973194-2974181 |
| 20:3730997-3732106 | 20:4248320-4250322 | 20:4470717-4471759 |
| 20:5065367-5066449 | 20:9507683-9509654 | 20:13665526-13666462 |
| 20:17227338-17228595 | 20:18502504-18503662 | 20:18748263-18749359 |
| 20:18813738-18814919 | 20:19342978-19343953 | 20:20072809-20074285 |
| 20:20364922-20366098 | 20:21101033-21104347 | 20:21104828-21107203 |
| 20:21396432-21398687 | 20:21507085-21508150 | 20:21508019-21509154 |
| 20:21509025-21510627 | 20:21515297-21518062 | 20:21518832-21519933 |
| 20:21519733-21520714 | 20:21520982-21523851 | 20:21704859-21707336 |
| 20:21713384-21715300 | 20:23034682-23035889 | 20:23451751-23453317 |
| 20:23928278-23929455 | 20:24134292-24135387 | 20:24468466-24472024 |
| 20:24917397-24918575 | 20:25080561-25084083 | 20:25147889-25149464 |
| 20:25249726-25251618 | 20:25985213-25986381 | 20:26209090-26210301 |
| 20:31412736-31413692 | 20:31472493-31474359 | 20:31993329-31995764 |
| 20:31996939-31999547 | 20:32005019-32006076 | 20:32030741-32031912 |
| 20:32049021-32049936 | 20:32205796-32206713 | 20:32600963-32602147 |
| 20:32877056-32878192 | 20:32984993-32986039 | 20:33086313-33087331 |
| 20:35246308-35247437 | 20:36331446-36332474 | 20:37250648-37251771 |
| 20:37676115-37677259 | 20:37933531-37935007 | 20:38259965-38261581 |
| 20:38722596-38728298 | 20:38902102-38903148 | 20:40682834-40684438 |
| 20:41365616-41367731 | 20:43187871-43191085 | 20:43539547-43541476 |
| 20:43914007-43916947 | 20:44110628-44111730 | 20:44230892-44231881 |
| 20:44291286-44292360 | 20:44811884-44812899 | 20:45230322-45231404 |
| 20:45316031-45317323 | 20:46056828-46057978 | 20:46058220-46059407 |
| 20:46117173-46118915 | 20:46151615-46152759 | 20:46514208-46515323 |
| 20:46582417-46583452 | 20:46623515-46624480 | 20:46810995-46812294 |
| 20:46901025-46902022 | 20:47747209-47748251 | 20:47973371-47974556 |
| 20:47985101-47986003 | 20:48385764-48387045 | 20:48643566-48644660 |
| 20:48740150-48741217 | 20:48806217-48807143 | 20:49206718-49207867 |
| 20:49218065-49219257 | 20:49346008-49347068 | 20:49567302-49568689 |
| 20:49786642-49787623 | 20:50009326-50010684 | 20:50064374-50065497 |
| 20:50354347-50355387 | 20:51008925-51010124 | 20:51036710-51037761 |
| 20:51192646-51193790 | 20:51540349-51541384 | 20:52103792-52106311 |
| 20:53345667-53346865 | 20:53607711-53608886 | 20:53942116-53943232 |
| 20:54150521-54151423 | 20:54471263-54472358 | 20:56004544-56006072 |
| 20:56576793-56577955 | 20:57459990-57461143 | 20:57514408-57515460 |
| 20:57690108-57691690 | 20:57693924-57695521 | 20:57938521-57939698 |
| 20:58183458-58184477 | 20:58245663-58246695 | 20:58465645-58467165 |
| 20:58513833-58515856 | 20:58542999-58543984 | 20:58578725-58579906 |
| 20:58755588-58756779 | 20:58797583-58798662 | 20:58816923-58818673 |
| 20:58818727-58819883 | 20:58822077-58823275 | 20:58876178-58877352 |
| 20:59410003-59411705 | 20:59622899-59624031 | 20:59719273-59721956 |
| 20:60024466-60025447 | 20:60074931-60076104 | 20:60084859-60085835 |
| 20:60085559-60086497 | 20:60453982-60455122 | 20:60642875-60644007 |
| 20:60975114-60976260 | 20:61339777-61340811 | 20:61391626-61392810 |
| 20:61501066-61502450 | 20:61602391-61603656 | 20:61678596-61680230 |
| 20:61870340-61871470 | 20:61928966-61930127 | 20:61959643-61960770 |
| 20:62206985-62208078 | 20:62474131-62477229 | 20:62555828-62557009 |
| 20:63004818-63006012 | 20:63176737-63179862 | 20:63193779-63194910 |
| 20:63318430-63320920 | 20:63341188-63342687 | 20:63360267-63362364 |
| 20:63424267-63425446 | 20:63577680-63579122 | 20:63749607-63750618 |
| 20:64047685-64050429 | 20:64097953-64099988 | 20:64105592-64107524 |
| 21:14757392-14758402 | 21:16033816-16034838 | 21:17725120-17726246 |
| 21:17920204-17921201 | 21:18244045-18246463 | 21:18752494-18753622 |
| 21:20996594-20999005 | 21:25337973-25339179 | 21:25886731-25888153 |
| 21:26815380-26816406 | 21:26836122-26837150 | 21:26843213-26847656 |
| 21:28626696-28627735 | 21:30598158-30599265 | 21:31251237-31252409 |
| 21:31557164-31560018 | 21:32584381-32586683 | 21:32658105-32659289 |
| 21:32929317-32930246 | 21:32957711-32959243 | 21:32982087-32983070 |
| 21:33022188-33024954 | 21:33024776-33027135 | 21:33056930-33058282 |
| 21:33121998-33123122 | 21:33124524-33125638 | 21:33131386-33132541 |
| 21:33166946-33167927 | 21:33170006-33171878 | 21:33554451-33555827 |
| 21:33896236-33897214 | 21:34187662-34188695 | 21:34312838-34313814 |
| 21:36542146-36543367 | 21:37005637-37006930 | 21:37198987-37200031 |
| 21:38391797-38392857 | 21:39011033-39011947 | 21:39499190-39500127 |
| 21:40385036-40385999 | 21:40511946-40513315 | 21:40576400-40577485 |
| 21:40846037-40847876 | 21:42136547-42137692 | 21:42394994-42396539 |
| 21:42568101-42569272 | 21:42595504-42596760 | 21:42692490-42693660 |
| 21:42730872-42731895 | 21:42774710-42775794 | 21:43785897-43787032 |
| 21:44349497-44351007 | 21:44453843-44455602 | 21:44625665-44626862 |
| 21:44695261-44696456 | 21:44708422-44710272 | 21:45015138-45016763 |
| 21:45305351-45306477 | 21:45317086-45318263 | 21:45351432-45352427 |
| 21:45361980-45363143 | 21:45370237-45371320 | 21:45590254-45591699 |
| 21:45734304-45735206 | 21:45787174-45788251 | 21:45871813-45872860 |
| 21:46031145-46032282 | 21:46112664-46113762 | 21:46179467-46180656 |
| 22:17175509-17176521 | 22:17209081-17210372 | 22:17547942-17549118 |
| 22:18121262-18122415 | 22:19613364-19614434 | 22:19639355-19640419 |
| 22:19723912-19725108 | 22:20148940-20150111 | 22:20240904-20242082 |
| 22:20642135-20643048 | 22:21707560-21708602 | 22:22035263-22036510 |
| 22:22507599-22508757 | 22:22558827-22560193 | 22:22923344-22924432 |
| 22:23035238-23036390 | 22:24494245-24495463 | 22:25281871-25283887 |
| 22:26168574-26170680 | 22:27792732-27793850 | 22:29473835-29474994 |
| 22:29479126-29480817 | 22:29581101-29582311 | 22:30210235-30211411 |
| 22:30235035-30236471 | 22:30967720-30968957 | 22:31063694-31064701 |
| 22:32175702-32176938 | 22:32996832-32997983 | 22:33057371-33059972 |
| 22:36430524-36431620 | 22:36702541-36704339 | 22:36990154-36991234 |
| 22:37068542-37069738 | 22:37334022-37335183 | 22:37824054-37825602 |
| 22:37847606-37848743 | 22:37857898-37858935 | 22:37985552-37986731 |
| 22:38401812-38403004 | 22:38457018-38458173 | 22:38763538-38765205 |
| 22:38835942-38837122 | 22:39656915-39659154 | 22:39661396-39662886 |
| 22:39893257-39895423 | 22:40677617-40678852 | 22:40990194-40991324 |
| 22:41026161-41027140 | 22:41067315-41068363 | 22:41351676-41352754 |
| 22:41698930-41700132 | 22:42421591-42422701 | 22:42552018-42553112 |
| 22:42556235-42557177 | 22:42565227-42566367 | 22:42692922-42694112 |
| 22:43242536-43243695 | 22:43255620-43256762 | 22:43836800-43837822 |
| 22:43861522-43863328 | 22:44404048-44405200 | 22:44914247-44915149 |
| 22:45004326-45005405 | 22:45095428-45096406 | 22:45461815-45462862 |
| 22:45540628-45541814 | 22:45709741-45710762 | 22:45865977-45868525 |
| 22:45893274-45894444 | 22:47065427-47066524 | 22:47196569-47197741 |
| 22:47215289-47216349 | 22:48308827-48309995 | 22:48488525-48489671 |
| 22:48594552-48595710 | 22:48615909-48617078 | 22:48683911-48685010 |
| 22:49449853-49450993 | 22:49458512-49459686 | 22:49521062-49522250 |
| 22:49614613-49615775 | 22:49630923-49632023 | 22:49733248-49734289 |
| 22:49763596-49765010 | 22:49765775-49766841 | 22:50048478-50049667 |
| 22:50266958-50268118 | 22:50734068-50735774 | 3:2303259-2304240 |
| 3:2900271-2901344 | 3:2980307-2981429 | 3:4798571-4799700 |
| 3:5095679-5096843 | 3:6861659-6863900 | 3:8316605-8317588 |
| 3:8321206-8322293 | 3:8745359-8746548 | 3:8767018-8768985 |
| 3:8990139-8991310 | 3:9551866-9553064 | 3:10815433-10816712 |
| 3:11014393-11016301 | 3:13037164-13038097 | 3:13040703-13042246 |
| 3:13054228-13055387 | 3:13072606-13074195 | 3:13101919-13103108 |
| 3:13203618-13204775 | 3:13266035-13266985 | 3:13281488-13284311 |
| 3:13833184-13834333 | 3:14494007-14495156 | 3:14724810-14725861 |
| 3:14809762-14812037 | 3:15420447-15421527 | 3:15516963-15517906 |
| 3:15777555-15778593 | 3:16427074-16428071 | 3:16472197-16473286 |
| 3:17143813-17144978 | 3:18099301-18100310 | 3:18837181-18838083 |
| 3:19749217-19750394 | 3:21750489-21751950 | 3:25555179-25556217 |
| 3:26621607-26625260 | 3:27720824-27725443 | 3:27727974-27731912 |
| 3:28574595-28577270 | 3:30720278-30721300 | 3:30894017-30895385 |
| 3:30897139-30898192 | 3:31452410-31453545 | 3:31500004-31501022 |
| 3:35677566-35678516 | 3:36763310-36765240 | 3:37196122-37197104 |
| 3:38647811-38649492 | 3:38899411-38900562 | 3:39265860-39266918 |
| 3:39443098-39444124 | 3:39466320-39467308 | 3:42199974-42201050 |
| 3:42264773-42266614 | 3:42408230-42409452 | 3:42410805-42411821 |
| 3:42773888-42774904 | 3:42864366-42865540 | 3:42984762-42985854 |
| 3:43037583-43038657 | 3:43055923-43057068 | 3:43217346-43218501 |
| 3:43994361-43999918 | 3:44240747-44241902 | 3:44570874-44571920 |
| 3:44710333-44713236 | 3:45387203-45388204 | 3:45795083-45796107 |
| 3:46265976-46266978 | 3:46371259-46372438 | 3:46516685-46517616 |
| 3:46731064-46732121 | 3:46831008-46831910 | 3:46854583-46855485 |
| 3:46881000-46883223 | 3:46898068-46899308 | 3:47365075-47366052 |
| 3:47478788-47479914 | 3:47535911-47537016 | 3:48588726-48589884 |
| 3:48594538-48595747 | 3:49543182-49544269 | 3:49718471-49719733 |
| 3:50466071-50467151 | 3:50658138-50659231 | 3:52360141-52361207 |
| 3:52390396-52391570 | 3:52671405-52672307 | 3:52998212-52999731 |
| 3:53370953-53372080 | 3:54087147-54088341 | 3:55480538-55483647 |
| 3:55483844-55484955 | 3:55485862-55490635 | 3:58093453-58094495 |
| 3:59450069-59451124 | 3:61570655-61571727 | 3:61627658-61628593 |
| 3:62367431-62375532 | 3:62377234-62379960 | 3:66475466-66476635 |
| 3:67774375-67775557 | 3:69359177-69360356 | 3:69998640-70000360 |
| 3:70747587-70748649 | 3:71752669-71755006 | 3:71892750-71893905 |
| 3:71930621-71931630 | 3:71962488-71963510 | 3:72259445-72260546 |
| 3:72727504-72728683 | 3:73623490-73626197 | 3:75618263-75620076 |
| 3:81985542-81986630 | 3:86989935-86992051 | 3:87088649-87090051 |
| 3:99637649-99639233 | 3:100185102-100186134 | 3:101251530-101252573 |
| 3:101501602-101502691 | 3:101928831-101929874 | 3:105393866-105394873 |
| 3:107881584-107882745 | 3:113157900-113158971 | 3:113210740-113211826 |
| 3:114034498-114035586 | 3:114319075-114320044 | 3:114504637-114505629 |
| 3:119499401-119500500 | 3:119558458-119559548 | 3:120284195-120286143 |
| 3:120396415-120397585 | 3:120780608-120781730 | 3:120907245-120908367 |
| 3:122885427-122886452 | 3:122921376-122923043 | 3:123321470-123322624 |
| 3:123375352-123376390 | 3:123995464-123996407 | 3:124093792-124095340 |
| 3:125356795-125358087 | 3:126213089-126214284 | 3:126489631-126490747 |
| 3:126503196-126504837 | 3:126571195-126573304 | 3:126729967-126731107 |
| 3:127134893-127136061 | 3:127282032-127282990 | 3:127550836-127551980 |
| 3:128485372-128486882 | 3:128492095-128494211 | 3:128898895-128899939 |
| 3:129524922-129525991 | 3:129527515-129529307 | 3:129531847-129533041 |
| 3:129547589-129548755 | 3:129973946-129976418 | 3:130348693-130349744 |
| 3:130431286-130432318 | 3:130559420-130560599 | 3:133673673-133675348 |
| 3:134648706-134649871 | 3:134650046-134651877 | 3:137642084-137643268 |
| 3:137760152-137761857 | 3:137764186-137766118 | 3:138362188-138363388 |
| 3:138450352-138451475 | 3:138936331-138937798 | 3:138943799-138944955 |
| 3:138945061-138948207 | 3:138949393-138951116 | 3:139538785-139540654 |
| 3:141050194-141053517 | 3:141066096-141067730 | 3:141098684-141099735 |
| 3:141662694-141663659 | 3:141776062-141777171 | 3:141980819-141981818 |
| 3:142829845-142830937 | 3:143130694-143131596 | 3:143313960-143315022 |
| 3:144585097-144586262 | 3:147354322-147355902 | 3:147355715-147356899 |
| 3:147356713-147357885 | 3:147358776-147360464 | 3:147369330-147371443 |
| 3:147377713-147378828 | 3:147380028-147381337 | 3:147384970-147386100 |
| 3:147386871-147390140 | 3:147390151-147391323 | 3:147391046-147395355 |
| 3:147395346-147396534 | 3:147396680-147397775 | 3:147397562-147399119 |
| 3:147404341-147412708 | 3:147412770-147414428 | 3:147418225-147419991 |
| 3:147421760-147425163 | 3:148366793-148367807 | 3:148697080-148698812 |
| 3:148854611-148855616 | 3:151012381-151013431 | 3:151084665-151087532 |
| 3:151424711-151425673 | 3:152466419-152467396 | 3:153880891-153882003 |
| 3:155078496-155080942 | 3:156290530-156291962 | 3:158094374-158096472 |
| 3:158098053-158099153 | 3:158102205-158104873 | 3:158572349-158573477 |
| 3:158718106-158719036 | 3:164340636-164341821 | 3:169763603-169764631 |
| 3:170417845-170420487 | 3:170584263-170586589 | 3:172447514-172450564 |
| 3:173914171-173915212 | 3:174146655-174147790 | 3:177624292-177625529 |
| 3:179654106-179655201 | 3:179671643-179672654 | 3:183588922-183589898 |
| 3:183591863-183592765 | 3:184671859-184673830 | 3:185070004-185070999 |
| 3:185071535-185072696 | 3:185156333-185157249 | 3:185559400-185560573 |
| 3:185734004-185735116 | 3:186360328-186363056 | 3:186456054-186456956 |
| 3:186635063-186636012 | 3:186831022-186832209 | 3:186992405-186993568 |
| 3:187701377-187702684 | 3:188929662-188930807 | 3:190320781-190321803 |
| 3:190434092-190435263 | 3:192513629-192516466 | 3:192726566-192728341 |
| 3:192915701-192916863 | 3:193902623-193903738 | 3:194350746-194351937 |
| 3:194397152-194398335 | 3:195105266-195106307 | 3:195809030-195811196 |
| 3:195872353-195873540 | 3:196225210-196226311 | 3:196337694-196338769 |
| 3:196513360-196514576 | 3:196532742-196534230 | 3:196604373-196605510 |
| 3:196708686-196709674 | 3:196989800-196990833 | 3:198109613-198110671 |
| 4:304925-306099 | 4:335923-338413 | 4:565878-566938 |
| 4:824288-825962 | 4:911444-913303 | 4:974028-975193 |
| 4:1001466-1003897 | 4:1154067-1155378 | 4:1170878-1172066 |
| 4:1197734-1198805 | 4:1309110-1310283 | 4:1337845-1339749 |
| 4:1347837-1349567 | 4:1401916-1403405 | 4:1403996-1405928 |
| 4:1407038-1408221 | 4:1408050-1409232 | 4:1413308-1414701 |
| 4:1606144-1607450 | 4:1632234-1633306 | 4:1654425-1655622 |
| 4:2424650-2425819 | 4:2434402-2435318 | 4:2694303-2695463 |
| 4:2976788-2977855 | 4:3026265-3027440 | 4:3641431-3643259 |
| 4:3667625-3668746 | 4:3669074-3670483 | 4:3680327-3681475 |
| 4:3724521-3725703 | 4:3766134-3767331 | 4:4170734-4172471 |
| 4:4672820-4673943 | 4:4854073-4856285 | 4:4857455-4859422 |
| 4:4860963-4862676 | 4:4863869-4868032 | 4:4871185-4872336 |
| 4:5018773-5020075 | 4:5456224-5457587 | 4:5459499-5460499 |
| 4:5572137-5573039 | 4:5622107-5623263 | 4:5889519-5891074 |
| 4:5891481-5892652 | 4:5973761-5975030 | 4:6221285-6222454 |
| 4:6244908-6246074 | 4:6472475-6473576 | 4:6663588-6664882 |
| 4:7111526-7113402 | 4:7148301-7149416 | 4:7183619-7184999 |
| 4:8290010-8291137 | 4:8353787-8354924 | 4:8370433-8371610 |
| 4:8387581-8388671 | 4:9450801-9451920 | 4:9780742-9782368 |
| 4:0445132-10446358 | 4:10456232-10458262 | 4:10461036-10462576 |
| 4:3523023-13524203 | 4:13526959-13530119 | 4:13534281-13536592 |
| 4:13538479-13539881 | 4:13545936-13547074 | 4:13968687-13969704 |
| 4:14583331-14584233 | 4:14995054-14995956 | 4:15420722-15422065 |
| 4:15787125-15788225 | 4:16360138-16361315 | 4:16474911-16475932 |
| 4:16726054-16727368 | 4:17780980-17782569 | 4:20250928-20255744 |
| 4:20883172-20884495 | 4:20983717-20984886 | 4:21947650-21949927 |
| 4:24794359-24795632 | 4:25050022-25050985 | 4:25088272-25089447 |
| 4:26182811-26183856 | 4:28402373-28403532 | 4:37951593-37952690 |
| 4:38765463-38766582 | 4:38783555-38784559 | 4:38795579-38796585 |
| 4:40377142-40378221 | 4:41255989-41257866 | 4:41744369-41745529 |
| 4:41745580-41747383 | 4:41747771-41749440 | 4:41749780-41752676 |
| 4:41864918-41866784 | 4:41866566-41868013 | 4:41872872-41874359 |
| 4:41878136-41879918 | 4:41879983-41883109 | 4:46992142-46994384 |
| 4:47030955-47032121 | 4:48482615-48484144 | 4:48954055-48955073 |
| 4:54099569-54102637 | 4:54108632-54110550 | 4:54374778-54375785 |
| 4:54656737-54658822 | 4:55819228-55820402 | 4:56654889-56657235 |
| 4:65667180-65668854 | 4:65668827-65671654 | 4:71382662-71383709 |
| 4:72567722-72570308 | 4:73835907-73836916 | 4:73870946-73871893 |
| 4:73998406-73999459 | 4:75991110-75992276 | 4:79324971-79326925 |
| 4:79407651-79408783 | 4:79825613-79826684 | 4:80184533-80186591 |
| 4:80268006-80269302 | 4:80996334-80997236 | 4:82740916-82742021 |
| 4:84495331-84496714 | 4: 84500661-84502575 | 4:85818375-85819277 |
| 4:86788579-86789550 | 4:91112509-91113516 | 4:94206004-94207003 |
| 4:95249884-95251024 | 4:98954677-98956393 | 4:99565034-99566052 |
| 4:100189400-100191734 | 4:100428260-100429304 | 4:100503787-100504826 |
| 4:103719425-103720615 | 4:105710298-105711301 | 4:106064959-106065989 |
| 4:107034012-107037992 | 4:107379817-107380829 | 4:110297818-110298920 |
| 4:110610201-110613373 | 4:110617588-110619442 | 4:110626043-110627336 |
| 4:110628160-110631789 | 4:110632241-110633433 | 4:110633224-110634930 |
| 4:110637234-110638780 | 4:110638722-110640713 | 4:110640762-110641943 |
| 4:112510132-112512206 | 4:112519864-112522213 | 4:112522616-112523803 |
| 4:116598694-116599874 | 4:117084697-117086153 | 4:121632157-121633322 |
| 4:121706290-121707192 | 4:121763894-121766134 | 4:121791135-121792212 |
| 4:122620442-122621651 | 4:133152634-133153814 | 4:134199444-134200407 |
| 4:134952363-134953515 | 4:139592585-139593630 | 4:140497520-140498964 |
| 4:140578873-140580044 | 4:140968079-140969403 | 4:141331452-141332566 |
| 4:146632424-146633581 | 4:146648200-146649364 | 4:146972265-146974136 |
| 4:151284772-151285745 | 4:152899739-152900762 | 4:153787696-153790514 |
| 4:153790416-153793517 | 4:154570209-154571209 | 4:154741463-154745269 |
| 4:155204376-155205454 | 4:155207478-155209649 | 4:155758346-155760808 |
| 4:156654135-156655181 | 4:157221006-157223351 | 4:159401644-159402790 |
| 4:160299658-160300692 | 4:161003443-161004547 | 4:163493293-163494195 |
| 4:164956413-164957667 | 4:165030755-165032468 | 4:165382809-165383821 |
| 4:165440445-165441637 | 4:165872080-165875660 | 4:169008106-169009485 |
| 4:169116005-169117074 | 4:169344749-169345784 | 4:171812010-171814942 |
| 4:173499709-173500881 | 4:173516206-173517721 | 4:173517876-173520046 |
| 4:173524451-173525649 | 4:173526334-173528251 | 4:173527954-173531914 |
| 4:174211149-174212487 | 4:176268719-176269769 | 4:176838157-176839059 |
| 4:177444382-177445439 | 4:182764231-182765256 | 4:182766776-182767903 |
| 4:182973789-182974969 | 4:183013485-183014629 | 4:183757638-183759275 |
| 4:184949778-184950873 | 4:184951073-184952274 | 4:185201265-185202412 |
| 4:185428900-185429885 | 4:186067338-186068455 | 4:186096170-186097347 |
| 4:187672230-187673273 | 4:187994922-187996548 | 4:188282222-188283405 |
| 4:189541017-189542174 | 4:190015149-190016337 | 4:190016877-190020155 |
| 4:190020602-190022176 | 5:142228-143619 | 5:154309-155472 |
| 5:163202-164323 | 5:171554-172754 | 5:191956-193084 |
| 5:193964-196079 | 5:320171-321314 | 5:585216-586285 |
| 5:616157-617287 | 5:1216365-1218048 | 5:1365861-1366763 |
| 5:1406337-1407969 | 5:1550109-1551264 | 5:1560059-1561154 |
| 5:1793528-1794676 | 5:1881821-1884979 | 5:1884765-1888401 |
| 5:1930103-1932539 | 5:1949597-1951179 | 5:2252162-2253474 |
| 5:2889667-2890802 | 5:3595991-3597187 | 5:4511518-4512768 |
| 5:4940753-4942956 | 5:5886646-5887844 | 5:6351412-6352314 |
| 5:6501402-6502514 | 5:6631157-6632424 | 5:7394260-7395162 |
| 5:7849200-7851852 | 5:10198169-10199207 | 5:10473992-10475264 |
| 5:10494556-10495713 | 5:10541063-10542234 | 5:10559155-10560189 |
| 5:10651189-10652782 | 5:10874946-10875986 | 5:10972980-10974080 |
| 5:11026457-11028358 | 5:11068382-11069514 | 5:11117356-11118373 |
| 5:11182857-11183939 | 5:11384018-11385977 | 5:11774304-11775453 |
| 5:13739435-13740451 | 5:15926615-15927642 | 5:16178452-16179624 |
| 5:16622622-16623702 | 5:17275751-17276763 | 5:17344983-17346003 |
| 5:18393036-18394039 | 5:19987380-19988971 | 5:20302186-20303352 |
| 5:31322884-31324000 | 5:31596493-31597623 | 5:31797892-31799891 |
| 5:31807737-31808788 | 5:32051883-32052901 | 5:32946212-32947352 |
| 5:33297293-33298430 | 5:33891468-33892941 | 5:35945468-35946661 |
| 5:35990299-35991857 | 5:36065877-36067440 | 5:36725397-36726415 |
| 5:37836053-37837220 | 5:37837084-37841276 | 5:40066253-40067437 |
| 5:41509231-41510576 | 5:42422918-42425569 | 5:42943759-42944772 |
| 5:44387808-44389835 | 5:53819208-53820399 | 5:54700635-54701628 |
| 5:54883112-54884848 | 5:55714398-55715489 | 5:58817559-58818654 |
| 5:63959381-63960659 | 5:63961281-63962584 | 5:64165171-64166941 |
| 5:64505429-64507766 | 5:65809600-65810551 | 5:71011284-71012379 |
| 5:71718395-71720190 | 5:73298036-73300555 | 5:73300599-73302035 |
| 5:73380847-73382967 | 5:73443224-73445450 | 5:74719738-74720767 |
| 5:75056930-75057832 | 5:75609874-75610902 | 5:76081567-76082743 |
| 5:76083640-76085826 | 5:76952522-76954667 | 5:77143864-77144925 |
| 5:77629022-77630847 | 5:77632418-77633552 | 5:77635633-77638148 |
| 5:77638040-77639841 | 5:77639934-77642657 | 5:77642984-77644305 |
| 5:77644069-77645615 | 5:77662735-77663869 | 5:77850583-77851917 |
| 5:77866562-77867464 | 5:77957488-77958707 | 5:79111259-79112454 |
| 5:79120648-79121801 | 5:80034525-80036253 | 5:80480647-80481707 |
| 5:81225229-81226310 | 5:82522410-82523565 | 5:88143853-88145017 |
| 5:88144868-88146063 | 5:88659207-88661661 | 5:88667073-88668367 |
| 5:88671432-88675422 | 5:88677328-88679315 | 5:88693598-88695851 |
| 5:88770079-88771149 | 5:88892503-88893405 | 5:90964920-90966006 |
| 5:91149982-91150884 | 5:93570331-93574305 | 5:93603868-93604960 |
| 5:94569022-94570104 | 5:96431569-96432741 | 5:96432720-96433826 |
| 5:100623206-100624240 | 5:103412979-103414007 | 5:111223216-111225258 |
| 5:114054924-114056961 | 5:115960909-115963981 | 5:116575969-116577233 |
| 5:123085850-123091638 | 5:123093169-123096025 | 5:123098098-123099362 |
| 5:125176016-125177075 | 5:128536933-128539702 | 5:128964527-128966235 |
| 5:132180912-132182069 | 5:132655687-132657003 | 5:132818940-132820444 |
| 5:133294511-133295568 | 5:133526324-133527409 | 5:133675443-133676564 |
| 5:135446836-135447738 | 5:135578092-135579849 | 5:135917725-135919018 |
| 5:135929469-135931573 | 5:136142889-136143898 | 5:136364817-136366047 |
| 5:138273937-138275221 | 5:138489366-138490281 | 5:138929428-138930480 |
| 5:139392395-139393613 | 5:139410633-139411813 | 5:139503347-139504475 |
| 5:139809153-139810346 | 5:139903164-139905430 | 5:140785587-140786652 |
| 5:140806448-140807572 | 5:141121137-141122320 | 5:141149408-141150689 |
| 5:141244772-141246272 | 5:141338265-141339427 | 5:141343207-141344475 |
| 5:141369646-141371147 | 5:141373369-141374426 | 5:141382211-141383371 |
| 5:141397248-141398374 | 5:141407412-141408896 | 5:141413301-141414482 |
| 5:141417074-141418251 | 5:141420229-141422285 | 5:141429969-141431466 |
| 5:141483949-141485867 | 5:141491421-141492616 | 5:142615022-142616221 |
| 5:143426229-143427370 | 5:144598177-144599308 | 5:145854818-145855943 |
| 5:146333593-146334770 | 5:146337943-146340450 | 5:146342308-146343505 |
| 5:147452192-147453327 | 5:147508876-147510727 | 5:147782017-147783179 |
| 5:149265039-149266047 | 5:149417670-149418742 | 5:150189405-150190972 |
| 5:150221782-150222975 | 5:150271346-150272521 | 5:150272795-150273905 |
| 5:151001415-151002441 | 5:151331977-151333022 | 5:151568433-151569396 |
| 5:151597248-151598280 | 5:151923835-151925598 | 5:153490356-153491505 |
| 5:154106984-154108049 | 5:154477307-154480893 | 5:154483291-154484560 |
| 5:157130785-157131952 | 5:157346087-157347059 | 5:157455943-157456996 |
| 5:157468610-157469677 | 5:158484647-158485731 | 5:158543602-158544603 |
| 5:158877575-158878504 | 5:159094835-159096435 | 5:159103917-159106551 |
| 5:159465859-159466931 | 5:159787260-159788260 | 5:161846752-161849446 |
| 5:162066979-162068627 | 5:164600543-164601752 | 5:164972161-164973157 |
| 5:166096939-166098652 | 5:167309593-167310687 | 5:167743297-167744199 |
| 5:168261684-168262823 | 5:168528737-168530500 | 5:168786063-168787205 |
| 5:169669145-169670239 | 5:169701865-169703434 | 5:169703253-169704368 |
| 5:169719047-169720131 | 5:169850220-169851270 | 5:169863647-169864823 |
| 5:169905504-169906677 | 5:170230760-170231662 | 5:170233135-170234293 |
| 5:170325576-170326657 | 5:170335820-170336873 | 5:170638992-170640125 |
| 5:171308403-171314501 | 5:171314512-171317543 | 5:171781843-171783022 |
| 5:172330862-172332011 | 5:172878334-172879597 | 5:173232128-173233652 |
| 5:173233430-173235712 | 5:173236188-173238031 | 5:173326153-173330576 |
| 5:174105853-174106915 | 5:174194601-174195632 | 5:174307675-174308727 |
| 5:175772807-175774024 | 5:175869162-175870232 | 5:176132706-176134161 |
| 5:176618555-176621084 | 5:176679617-176681022 | 5:177383260-177384755 |
| 5:178055248-178056410 | 5:178245797-178246872 | 5:178255432-178256552 |
| 5:178463273-178464450 | 5:178518804-178519914 | 5:178546179-178547334 |
| 5:178654240-178655202 | 5:178945688-178946814 | 5:178982497-178983641 |
| 5:178985957-178988103 | 5:179033774-179034754 | 5:179050549-179051705 |
| 5:179160427-179161502 | 5:179195445-179196587 | 5:179211430-179212627 |
| 5:179223264-179225026 | 5:179271489-179272668 | 5:179360854-179361993 |
| 5:180014833-180015850 | 5:180034764-180035735 | 5:180203980-180204882 |
| 5:180306865-180308011 | 5:180605966-180607145 | 5:180648293-180649475 |
| 5:180658671-180659858 | 5:180865208-180866128 | 5:181059012-181060476 |
| 5:181287661-181288774 | 6:183237-184226 | 6:230391-231476 |
| 6:362890-364632 | 6:470123-471274 | 6:1382247-1385127 |
| 6:1392856-1394385 | 6:1554736-1555860 | 6:1602311-1603858 |
| 6:1605084-1606661 | 6:1607672-1609021 | 6:1864262-1865259 |
| 6:2376553-2377917 | 6:2448941-2449952 | 6:2459513-2460670 |
| 6:2509328-2510464 | 6:3217278-3218329 | 6:3239199-3240304 |
| 6:3269225-3270352 | 6:4781761-4782663 | 6:5993551-5996779 |
| 6:5998365-6000045 | 6:6001377-6005166 | 6:6050661-6051745 |
| 6:6534049-6535189 | 6:7777663-7778801 | 6:7988354-7989402 |
| 6:10881098-10884507 | 6:11007897-11008866 | 6:12663930-12664978 |
| 6:14003804-14004777 | 6:14709613-14710515 | 6:14798174-14799311 |
| 6:14965122-14966130 | 6:15142996-15144005 | 6:17015651-17016841 |
| 6:17354421-17355396 | 6:21663826-21666250 | 6:21665974-21667678 |
| 6:23174349-23175338 | 6:24442824-24443998 | 6:25709671-25711035 |
| 6:25726205-25727364 | 6:27210763-27211848 | 6:27259893-27261544 |
| 6:27267474-27268691 | 6:27288093-27289175 | 6:27290163-27291909 |
| 6:27473598-27474778 | 6:27557878-27559046 | 6:27565498-27566959 |
| 6:27591548-27592733 | 6:27679336-27681416 | 6:28206903-28209042 |
| 6:28541932-28543355 | 6:28583009-28584393 | 6:28589423-28590611 |
| 6:28709142-28710493 | 6:28729441-28730641 | 6:28765045-28766466 |
| 6:28769198-28770723 | 6:28772682-28773784 | 6:28774690-28776518 |
| 6:28813221-28814267 | 6:28824883-28826254 | 6:28861936-28863576 |
| 6:28921317-28922417 | 6:28987880-28989077 | 6:28994542-28996572 |
| 6:29004023-29005138 | 6:29553128-29554585 | 6:29557701-29559345 |
| 6:29849923-29851054 | 6:30007041-30008162 | 6:30189766-30190809 |
| 6:30452619-30453791 | 6:30983689-30984849 | 6:31573121-31574160 |
| 6:32052149-32053199 | 6:32054314-32056597 | 6:32066094-32067701 |
| 6:32096165-32097597 | 6:32147736-32149902 | 6:32209292-32211477 |
| 6:32215923-32218723 | 6:32218565-32221939 | 6:32321667-32323165 |
| 6:33085656-33086675 | 6:33116396-33117742 | 6:33171301-33172316 |
| 6:33361694-33362682 | 6:33422679-33423962 | 6: 33789285-33790381 |
| 6:33803559-33804691 | 6:33892247-33894202 | 6:33910069-33911622 |
| 6:36839848-36841298 | 6:37010041-37011113 | 6:37350382-37351525 |
| 6:37535742-37536932 | 6:37538753-37539926 | 6:37648241-37649412 |
| 6:37663523-37664897 | 6:38714607-38715975 | 6:38722272-38723339 |
| 6:39313508-39314705 | 6:39899547-39900944 | 6:41344601-41345503 |
| 6:41560009-41561257 | 6:41637723-41639297 | 6:42175461-42176439 |
| 6:42726677-42727857 | 6:42770680-42771792 | 6:43284546-43286248 |
| 6:43335986-43336896 | 6:44313436-44314463 | 6:44767962-44768979 |
| 6:45423639-45424836 | 6:47476268-47477170 | 6:48067951-48069532 |
| 6:48701241-48702588 | 6:49636160-49637180 | 6:49785548-49786450 |
| 6:49787987-49789137 | 6:50706349-50707528 | 6:50707311-50708467 |
| 6:50708384-50709568 | 6:50722675-50723878 | 6:50723845-50725470 |
| 6:50818403-50819534 | 6:50821111-50822303 | 6:50822947-50824298 |
| 6:50849093-50850594 | 6:50850562-50851858 | 6:52184884-52186250 |
| 6:53239413-53240421 | 6:53826944-53827991 | 6:55173843-55175159 |
| 6:57039659-57040796 | 6:57044987-57046014 | 6:58072815-58074097 |
| 6:70270544-70271622 | 6:70650837-70651929 | 6:72182009-72183197 |
| 6:72252318-72253349 | 6:73156716-73157891 | 6:73314259-73315436 |
| 6:75084563-75085763 | 6:77466164-77467668 | 6:83707402-83710429 |
| 6:83852507-83854483 | 6:84761950-84765652 | 6:84765919-84767803 |
| 6:84772639-84775707 | 6:87094775-87095810 | 6:87409614-87410672 |
| 6:88165001-88168259 | 6:88169118-88170626 | 6:89133532-89134578 |
| 6:89561912-89563345 | 6:89616128-89617092 | 6:89638871-89640105 |
| 6:89924862-89926112 | 6:90195074-90196089 | 6:90401519-90402675 |
| 6:93416362-93420467 | 6:99602030-99604546 | 6:99607631-99609837 |
| 6:99618693-99619893 | 6:99992970-99994726 | 6:100446184-100449521 |
| 6:100453780-100454878 | 6:100455120-100456535 | 6:100457905-100459370 |
| 6:100460966-100462151 | 6:100463057-100465669 | 6:100465522-100468457 |
| 6:100468589-100469930 | 6:102907334-102908448 | 6:104952408-104953882 |
| 6:105135672-105138196 | 6:105173967-105175049 | 6:108163270-108167677 |
| 6:108169098-108170498 | 6:109423668-109424805 | 6:109977712-109979613 |
| 6:110054163-110055216 | 6:110250184-110251253 | 6:110420082-110421125 |
| 6:111047962-111049040 | 6:112053318-112055259 | 6:112346748-112347820 |
| 6:117262787-117264033 | 6:117264011-117267017 | 6:119149561-119150704 |
| 6:124961151-124964011 | 6:125316274-125317449 | 6:127514244-127517134 |
| 6:129681680-129682753 | 6:130012410-130013583 | 6:131059164-131060241 |
| 6:132374789-132375914 | 6:132904421-132905623 | 6:133239917-133244018 |
| 6:133853667-133855958 | 6:133888444-133890666 | 6:133892930-133894397 |
| 6:134035897-134037038 | 6:135487824-135488847 | 6:136208395-136209491 |
| 6:137157977-137159113 | 6:137201753-137202846 | 6:137452005-137452997 |
| 6:137487128-137489085 | 6:137492369-137498728 | 6:142088269-142089560 |
| 6:143743498-143744551 | 6:144233911-144234858 | 6:144856124-144857172 |
| 6:144897611-144898644 | 6:145042133-145043293 | 6:146027186-146029898 |
| 6:146433889-146435352 | 6:146901722-146902827 | 6:149312521-149313520 |
| 6:149428842-149430855 | 6:149910287-149911437 | 6:149954637-149955722 |
| 6:149964770-149966248 | 6:150866771-150867711 | 6:151239446-151242093 |
| 6:151349596-151351142 | 6:151803360-151805422 | 6:151806672-151809200 |
| 6:152123769-152124899 | 6:152333683-152334688 | 6:152753651-152754597 |
| 6:154038607-154040323 | 6:154165733-154166909 | 6:155210691-155211800 |
| 6:157182484-157183495 | 6:157335234-157336280 | 6:157491409-157492592 |
| 6:157531904-157532922 | 6:157552211-157553231 | 6:158023231-158024385 |
| 6:158150334-158151390 | 6:158730036-158731273 | 6:159130861-159131960 |
| 6:159167081-159168098 | 6:160678517-160679808 | 6:161265723-161266850 |
| 6:161407903-161408903 | 6:161442097-161443278 | 6:162262915-162264005 |
| 6:163193031-163194060 | 6:163239694-163241085 | 6:163242848-163244006 |
| 6:163297343-163298531 | 6:163333611-163334738 | 6:163590692-163591892 |
| 6:163630921-163632102 | 6:163713830-163715001 | 6:163969253-163970325 |
| 6:165282784-165283949 | 6:165332942-165334126 | 6:165828734-165829820 |
| 6:166008093-166009816 | 6:166165118-166170499 | 6:166306670-166308267 |
| 6:166442801-166443984 | 6:166530111-166531394 | 6:166771056-166772010 |
| 6:166778525-166779652 | 6:168101069-168102659 | 6:168211359-168213201 |
| 6:168232264-168233267 | 6:168409796-168411977 | 6:168546942-168548080 |
| 6:169140131-169141285 | 6:169156786-169157872 | 6:169236930-169238108 |
| 6:169800977-169802202 | 6:170018798-170019956 | 6:170098913-170100576 |
| 6:170295940-170297135 | 7:57983-59023 | 7:133343-135081 |
| 7:179603-180999 | 7:211960-213139 | 7:212872-214303 |
| 7:227447-228627 | 7:640650-642192 | 7:809997-811117 |
| 7:839647-840829 | 7:908328-909526 | 7:1182484-1183625 |
| 7:1228949-1231940 | 7:1234021-1236591 | 7:1236918-1238699 |
| 7:1238937-1240187 | 7:1240016-1241180 | 7:1278724-1279862 |
| 7:1314557-1315871 | 7:1418548-1419714 | 7:1528839-1530019 |
| 7:1616412-1617482 | 7:1654561-1655866 | 7:1758587-1759793 |
| 7:1767955-1770294 | 7:1779088-1780222 | 7:1921731-1923353 |
| 7:1930711-1932113 | 7:1957107-1958203 | 7:2026861-2028021 |
| 7:2062306-2064023 | 7:2569539-2570496 | 7:2699906-2701819 |
| 7:2761080-2762148 | 7:2875438-2876619 | 7:3088665-3089756 |
| 7:3134901-3136001 | 7:3303498-3304514 | 7:3378547-3380231 |
| 7:3643955-3645087 | 7:3970541-3972018 | 7:4039848-4040934 |
| 7:4816674-4817984 | 7:4820546-4821699 | 7:4845307-4846612 |
| 7:4895362-4896521 | 7:5843513-5844607 | 7:5871592-5872767 |
| 7:6283701-6284862 | 7:6557109-6558310 | 7:6663422-6665014 |
| 7:6848951-6850079 | 7:7529055-7530077 | 7:7565079-7566164 |
| 7:8434976-8436158 | 7: 8440575-8444327 | 7:10779151-10780801 |
| 7:11251608-11252660 | 7:11330926-11331925 | 7:12570881-12572207 |
| 7:16914457-16915552 | 7:19105815-19109163 | 7:19109783-19110939 |
| 7:19110984-19113313 | 7:19115711-19120487 | 7:19143019-19146253 |
| 7:19710747-19711724 | 7:19772254-19774158 | 7:20560233-20561260 |
| 7:20659098-20660189 | 7:20664653-20665839 | 7:20784553-20787034 |
| 7:20793291-20795339 | 7:21169021-21170136 | 7:22095810-22096884 |
| 7:22596191-22597196 | 7:23205487-23206529 | 7:23247039-23248523 |
| 7:23708216-23709166 | 7:24283045-24285974 | 7:24521084-24522088 |
| 7:25855680-25856809 | 7:25857685-25860113 | 7:25859948-25861723 |
| 7:26244404-26245567 | 7:26636121-26637922 | 7:27101599-27103168 |
| 7:27151844-27153222 | 7:27154526-27156005 | 7:27157709-27158857 |
| 7:27166351-27167513 | 7:27188811-27190119 | 7:27212326-27214357 |
| 7:27219900-27221417 | 7:27240508-27243197 | 7:27244334-27246101 |
| 7:27246024-27247158 | 7:27252407-27253586 | 7:27863520-27864666 |
| 7:28279294-28280645 | 7:28522390-28523575 | 7:28539449-28540517 |
| 7:28554298-28555316 | 7:28641914-28643782 | 7:28797939-28799141 |
| 7:28807769-28808812 | 7:29968433-29969472 | 7:30631249-30632268 |
| 7:30681279-30683392 | 7:33175677-33176770 | 7:33493665-33494621 |
| 7:33790720-33791835 | 7:33903299-33904385 | 7:35252831-35256067 |
| 7:35260864-35262792 | 7:35430132-35431263 | 7:35454299-35455484 |
| 7:35569420-35570531 | 7:36276802-36277941 | 7:36280133-36281201 |
| 7:36281753-36283036 | 7:36513083-36514244 | 7:36581778-36582929 |
| 7:36600204-36601357 | 7:36632553-36633636 | 7:36861793-36862836 |
| 7:36879602-36880618 | 7:36985647-36986844 | 7:37046834-37047736 |
| 7:37224453-37225562 | 7:37248481-37249512 | 7:37795691-37796741 |
| 7:38430037-38430939 | 7:38462274-38463344 | 7:38558722-38559880 |
| 7:38975507-38976999 | 7:39356912-39357961 | 7:39411500-39412428 |
| 7:39919612-39920555 | 7:41096437-41098018 | 7:41581327-41582438 |
| 7:41681595-41682591 | 7:42197110-42198144 | 7:42227150-42228709 |
| 7:42337319-42338362 | 7:43444913-43446122 | 7:43810220-43811363 |
| 7:44145445-44146637 | 7:44218540-44220048 | 7:44276404-44277581 |
| 7:45161503-45162657 | 7:45186362-45187420 | 7:45573237-45576478 |
| 7:47227531-47228672 | 7:47534001-47535052 | 7:47846176-47847162 |
| 7:47860510-47861576 | 7:48090149-48091158 | 7:48154747-48155800 |
| 7:50211342-50212469 | 7:50358986-50360182 | 7:50370485-50372316 |
| 7:53218636-53219986 | 7:54541346-54543080 | 7:54888013-54889296 |
| 7:55222692-55223814 | 7:55472532-55473630 | 7:63900180-63901583 |
| 7:63925021-63927031 | 7:64352156-64353457 | 7:64494808-64496005 |
| 7:64568957-64570943 | 7:64882018-64883536 | 7:65954492-65955489 |
| 7:66504080-66505398 | 7:66505518-66506963 | 7:67943230-67944331 |
| 7:69367640-69368758 | 7:70132244-70133198 | 7:70426070-70427161 |
| 7:70807346-70808385 | 7:72263576-72264670 | 7:73311908-73313541 |
| 7:73328649-73329820 | 7:76276181-76277549 | 7:78040099-78041264 |
| 7:80644340-80645334 | 7:80921141-80922191 | 7:81725489-81726527 |
| 7:86643144-86645725 | 7:86944541-86945686 | 7:87218215-87219330 |
| 7:87623056-87624189 | 7:87910618-87911529 | 7:88793814-88794919 |
| 7:90118048-90120281 | 7:92608131-92609385 | 7:93441777-93442814 |
| 7:93573804-93576023 | 7:96992707-96993877 | 7:96995734-96998848 |
| 7:97002474-97004905 | 7:97012640-97013983 | 7:97020501-97025267 |
| 7:97731153-97734352 | 7:97938786-97939948 | 7:97972718-97973867 |
| 7:98239769-98240906 | 7:98353577-98354703 | 7:98470043-98471427 |
| 7:98616094-98617920 | 7:100400165-100401273 | 7:100488244-100489398 |
| 7:100633503-100634892 | 7:100890801-100891974 | 7:101156527-101157643 |
| 7:101164227-101165806 | 7:101179397-101181275 | 7:101299285-101300449 |
| 7:101302246-101304085 | 7:101333559-101334692 | 7:101952266-101953575 |
| 7:102012555-102013704 | 7:108456449-108457613 | 7:111197874-111198856 |
| 7:116261941-116262940 | 7:117321913-117324541 | 7:118213624-118215249 |
| 7:121328248-121329571 | 7:122299188-122301503 | 7:127250544-127251851 |
| 7:127346448-127347540 | 7:128030340-128031808 | 7:128031511-128032781 |
| 7:128134332-128135464 | 7:128167188-128169326 | 7:128909968-128911364 |
| 7:128915292-128916421 | 7:129060597-129061571 | 7:129213442-129214735 |
| 7:130291617-130292732 | 7:130876238-130877284 | 7:132082466-132083464 |
| 7:132648370-132649391 | 7:134982277-134983243 | 7:135230982-135232096 |
| 7:135245432-135247357 | 7:135710716-135711912 | 7:136253723-136254801 |
| 7:136868045-136872030 | 7:137775100-137776205 | 7:137852084-137853051 |
| 7:138645228-138646268 | 7:138665353-138666540 | 7:138831090-138832147 |
| 7:139505054-139506246 | 7:140526808-140528123 | 7:141929428-141930583 |
| 7:142859951-142860998 | 7:142861598-142863662 | 7:142863683-142864884 |
| 7:142870460-142871895 | 7:142938726-142939872 | 7:143882044-143883645 |
| 7:143884828-143886008 | 7:148539882-148540927 | 7:149461774-149462871 |
| 7:149692243-149693430 | 7:149767297-149768971 | 7:149789671-149791149 |
| 7:149801334-149802367 | 7:149819979-149820967 | 7:149833344-149834465 |
| 7:151389971-151391716 | 7:151426008-151427690 | 7:151431566-151432705 |
| 7:151434174-151435189 | 7:151607118-151608201 | 7:151754577-151755627 |
| 7:152595232-152596246 | 7:152924726-152925948 | 7:153886451-153888362 |
| 7:154752809-154753950 | 7:154793677-154794869 | 7:154889245-154890320 |
| 7:154892332-154893839 | 7:155069725-155071289 | 7:155453178-155455953 |
| 7:155461971-155463171 | 7:155465114-155469300 | 7:155548452-155549651 |
| 7:156641151-156642160 | 7:157058281-157059478 | 7:157077943-157079095 |
| 7:157313265-157314441 | 7:157407106-157408242 | 7:157433775-157435135 |
| 7:157521028-157522604 | 7:157685216-157686401 | 7:157687948-157690119 |
| 7:157691266-157694616 | 7:157736065-157737229 | 7:157790812-157791986 |
| 7:157812278-157813570 | 7:157834201-157835383 | 7:157866206-157868188 |
| 7:157925142-157926255 | 7:157985320-157988065 | 7:158584961-158585974 |
| 7:158880695-158881844 | 7:159026251-159027435 | 7:159065243-159066984 |
| 7:159095582-159096676 | 7:159143300-159146299 | 8:495065-496501 |
| 8:749699-751188 | 8:752304-754743 | 8:763052-764225 |
| 8:860277-862028 | 8:902585-905567 | 8:917905-918998 |
| 8:980802-981704 | 8:1036074-1037232 | 8:1103970-1105461 |
| 8:1357220-1358291 | 8:1451523-1452690 | 8:1519857-1521023 |
| 8:1552024-1555161 | 8:1621147-1623390 | 8:1665290-1666559 |
| 8:1676064-1678016 | 8:1690790-1691898 | 8:2011502-2012859 |
| 8:2032382-2034216 | 8:2078348-2079525 | 8:2129473-2130662 |
| 8:2139837-2141210 | 8:2178626-2179791 | 8:2562132-2563261 |
| 8:2894763-2895836 | 8:3646008-3647177 | 8:4034607-4035683 |
| 8:5670768-5671918 | 8:6296241-6297309 | 8 6407509-6408559 |
| 8:6779905-6781053 | 8:8781352-8783016 | 8:8852171-8853263 |
| 8:8877648-8879052 | 8:9898066-9899268 | 8:9902790-9907715 |
| 8:9907583-9908723 | 8:9932693-9934498 | 8:10291446-10292435 |
| 8:10477194-10478333 | 8:11109992-11111158 | 8:11682574-11683691 |
| 8:11707168-11709185 | 8:11709081-11710791 | 8:12692502-12693692 |
| 8:12765667-12766821 | 8:13011541-13012664 | 8:13051585-13052722 |
| 8:13438100-13439235 | 8:13491434-13492384 | 8:15236376-15239003 |
| 8:17412701-17414381 | 8:20614406-20616405 | 8:21772419-21773459 |
| 8:22231295-22232719 | 8:23704844-23708228 | 8:23708795-23710821 |
| 8:24912273-24914197 | 8:24915033-24916218 | 8:25392147-25393275 |
| 8:26039085-26043057 | 8:26042946-26046275 | 8:26046098-26049211 |
| 8:26668154-26669330 | 8:27122319-27123517 | 8:27394734-27395721 |
| 8:27469255-27470414 | 8:27591630-27592751 | 8:29955523-29956562 |
| 8:31030784-31032004 | 8:31252303-31253392 | 8:31638530-31641090 |
| 8:33337352-33338395 | 8:33722084-33723232 | 8:37964843-37967239 |
| 8:38665442-38666485 | 8:38867523-38868724 | 8:39106921-39108332 |
| 8:41100925-41101997 | 8:41566403-41567585 | 8:43561326-43562709 |
| 8:47763739-47764937 | 8:48734431-48736411 | 8:48888871-48889970 |
| 8:49909011-49912517 | 8:52938012-52942386 | 8:53250460-53252479 |
| 8:53876104-53878555 | 8:53879001-53882892 | 8:54457029-54459396 |
| 8:54465918-54468040 | 8:56112537-56114338 | 8:56437938-56439077 |
| 8:56446066-56447434 | 8:56929316-56930264 | 8:60875930-60877930 |
| 8:61139258-61140446 | 8:61720510-61721520 | 8:62247246-62248545 |
| 8:63703828-63705051 | 8:64368337-64372572 | 8:64372709-64373881 |
| 8:64373867-64375015 | 8:64376057-64380536 | 8:64575056-64582472 |
| 8:64585429-64587062 | 8:65244922-65246096 | 8:65560127-65561277 |
| 8:66458283-66459446 | 8:66463870-66464828 | 8:66470903-66471904 |
| 8:66960532-66963806 | 8:67731517-67733039 | 8:70065598-70066714 |
| 8:70069217-70072920 | 8:70564270-70565380 | 8:71841640-71846146 |
| 8:72032450-72033557 | 8:72154062-72155252 | 8:73291363-73292852 |
| 8:73345935-73346923 | 8:73435179-73436186 | 8:79610505-79611692 |
| 8:80265595-80266651 | 8:80316960-80318080 | 8:80499124-80500131 |
| 8:86661361-86662527 | 8:92101001-92104323 | 8:92642116-92643273 |
| 8:92667014-92668015 | 8:93017217-93019064 | 8:93495355-93496805 |
| 8:96144030-96146244 | 8:96153260-96154663 | 8:96156942-96162090 |
| 8:96402197-96403339 | 8:96492234-96495033 | 8:97129637-97130781 |
| 8:97276231-97277253 | 8:98356269-98357220 | 8:98399267-98400419 |
| 8:98407477-98408489 | 8:98947385-98950236 | 8:98950830-98951892 |
| 8:98973066-98975223 | 8:100105553-100107559 | 8:101166763-101167863 |
| 8:103139986-103142108 | 8:104363004-104364140 | 8:104418590-104420276 |
| 8:104466229-104467723 | 8:104752108-104753149 | 8:105825119-105826167 |
| 8:106215169-106216368 | 8:106769687-106770957 | 8:108081211-108084523 |
| 8:108786750-108788651 | 8:109973300-109976409 | 8:110829443-110830600 |
| 8:113432275-113434142 | 8:115467490-115468558 | 8:115547077-115548148 |
| 8:116355000-116355977 | 8:116642218-116643262 | 8:120375545-120376599 |
| 8:122956208-122957312 | 8:123148544-123149644 | 8:123235114-123236448 |
| 8:123355156-123356285 | 8:124270478-124272253 | 8:124920146-124921177 |
| 8:125359109-125360267 | 8:125543061-125544110 | 8:127480664-127481731 |
| 8:127870364-127871398 | 8:129241336-129242761 | 8:129290612-129291514 |
| 8:129344305-129345353 | 8:129515475-129516377 | 8:129542452-129543507 |
| 8:129728407-129729456 | 8:129731528-129732430 | 8:129819434-129820589 |
| 8:129963118-129964283 | 8:130681794-130682803 | 8:131039863-131043061 |
| 8:132319176-132320218 | 8:132561014-132562214 | 8:132567521-132568693 |
| 8:132661155-132662206 | 8:132733206-132734190 | 8:132915053-132916084 |
| 8:132984614-132985647 | 8:133039580-133040622 | 8:133087512-133088570 |
| 8:133106867-133108019 | 8:133130643-133131862 | 8:133195757-133196806 |
| 8:133462638-133463757 | 8:133509865-133510935 | 8:133858575-133859605 |
| 8:134543724-134544829 | 8:134696155-134697309 | 8:138495990-138498061 |
| 8:138804078-138805146 | 8:139601405-139602714 | 8:139605484-139606386 |
| 8:139611968-139613065 | 8:139618271-139619446 | 8:139619976-139621181 |
| 8:139701750-139705580 | 8:139739017-139740202 | 8:140492440-140493491 |
| 8:140764030-140765144 | 8:141222877-141223926 | 8:141276609-141277725 |
| 8:141420638-141421788 | 8:141424962-141426144 | 8:141496275-141497308 |
| 8:141526602-141527504 | 8:141546397-141547479 | 8:141621793-141623067 |
| 8:141784672-141786386 | 8:141902367-141903557 | 8:141988265-141989411 |
| 8:142055335-142056289 | 8:142070756-142071939 | 8:142129023-142130221 |
| 8:142242797-142244132 | 8:142431538-142432672 | 8:142453012-142454186 |
| 8:142463634-142465184 | 8:142589645-142591466 | 8:142833523-142836749 |
| 8:143031549-143032705 | 8:143121473-143122661 | 8:143154515-143155716 |
| 8:143197716-143198828 | 8:143264015-143266221 | 8:143281574-143283418 |
| 8:143284501-143285805 | 8:143344440-143345462 | 8:143396930-143398038 |
| 8:143460738-143461984 | 8:143905258-143906439 | 8:144048365-144051202 |
| 8:144335528-144336722 | 8:144699751-144701259 | 8:144703886-144705621 |
| 9:842202-843777 | 9:4289990-4291048 | 9:5307374-5308276 |
| 9:6552061-6553093 | 9:7246035-7247079 | 9:7961705-7962721 |
| 9:13896495-13897525 | 9:14346279-14347415 | 9:14976477-14977608 |
| 9:14992866-14994068 | 9:17905517-17907750 | 9:18260253-18261155 |
| 9:19787509-19789897 | 9:19932131-19933061 | 9:21967675-21968824 |
| 9:21970655-21971910 | 9:22004700-22006163 | 9:23823579-23824753 |
| 9:23829766-23830917 | 9:27283639-27284808 | 9:33511126-33512171 |
| 9:34577418-34578574 | 9:35975665-35976780 | 9:37666355-37667401 |
| 9:68584724-68585635 | 9:70536736-70537862 | 9:74499859-74501035 |
| 9:77012981-77014023 | 9:77014220-77015770 | 9:77015518-77017885 |
| 9:77017861-77021564 | 9:77021939-77023128 | 9:79692719-79693665 |
| 9:80062463-80063415 | 9:83701384-83702451 | 9:87860151-87861135 |
| 9:88532009-88533174 | 9:88672909-88674032 | 9:88932121-88933246 |
| 9:89256198-89257313 | 9:89648538-89649635 | 9:90041806-90042948 |
| 9:90515180-90516122 | 9:90612905-90614097 | 9:92806741-92808133 |
| 9:93345586-93347192 | 9:93947034-93948070 | 9:93951416-93953988 |
| 9:94705076-94706381 | 9:94798583-94799584 | 9:94853289-94854311 |
| 9:95181397-95182426 | 9:95348495-95350599 | 9:96209665-96210714 |
| 9:96719213-96720412 | 9:96876809-96878247 | 9:98311998-98313047 |
| 9:98555892-98557018 | 9:98706387-98710282 | 9:98780099-98781199 |
| 9:99020202-99021331 | 9:99530953-99532113 | 9:99828424-99829551 |
| 9:100353305-100354287 | 9:101613038-101614081 | 9:101736840-101739437 |
| 9:108919368-108920385 | 9:108934665-108935816 | 9:109166671-109167995 |
| 9:110047520-110049123 | 9:110049119-110050277 | 9:110339997-110340899 |
| 9:111097725-111099450 | 9:113011007-113012022 | 9:113796561-113797611 |
| 9:114657588-114658632 | 9:116959373-116960527 | 9:117744362-117745736 |
| 9:120299478-120300380 | 9:121203124-121204026 | 9:122126142-122127323 |
| 9:122218625-122220706 | 9:122659376-122660423 | 9:122788718-122789909 |
| 9:122797410-122799063 | 9:123959810-123960910 | 9:123970874-123972040 |
| 9:124009758-124010833 | 9:124016422-124018184 | 9:124022219-124023396 |
| 9:124128241-124129348 | 9:124136617-124137741 | 9:125426987-125428590 |
| 9:126576227-126577129 | 9:126610101-126611291 | 9:126623346-126626020 |
| 9:127048071-127049087 | 9:127186865-127187797 | 9:128201644-128202829 |
| 9:128428886-128430081 | 9:128817247-128818427 | 9:129619572-129621310 |
| 9:129624052-129625943 | 9:130308642-130309690 | 9:130658972-130662507 |
| 9:131816790-131817692 | 9:131835974-131837170 | 9:132163847-132165041 |
| 9:132200016-132201140 | 9:132431035-132432055 | 9:132473863-132474918 |
| 9:132637134-132638319 | 9:132708305-132709488 | 9:132885826-132887011 |
| 9:133032144-133033192 | 9:133488710-133489801 | 9:133691487-133692618 |
| 9:133699947-133701076 | 9:134204861-134205989 | 9:134485411-134486570 |
| 9:134500852-134502040 | 9:134556206-134557385 | 9:134582195-134583345 |
| 9:134606948-134607993 | 9:134652768-134654021 | 9:134661383-134662423 |
| 9:134662557-134664377 | 9:134744607-134745799 | 9:134780603-134782348 |
| 9:134808248-134810080 | 9:134830815-134831879 | 9:134834944-134836066 |
| 9:134837908-134840596 | 9:134872433-134873623 | 9:134882600-134883758 |
| 9:134931754-134932925 | 9:135074645-135076405 | 9:135119894-135121302 |
| 9:135544753-135545907 | 9:135625998-135627048 | 9:135655675-135656774 |
| 9:135735786-135737078 | 9:135784185-135785938 | 9:135804790-135806688 |
| 9:135864625-135865764 | 9:135974539-135975684 | 9:136186826-136188017 |
| 9:136198550-136200131 | 9:136332650-136333886 | 9:136561543-136562624 |
| 9:136985022-136986351 | 9:136997680-136998672 | 9:137009114-137010285 |
| 9:137138557-137139759 | 9:137373736-137374916 | 9:137522497-137523690 |
| 9:137542054-137543150 | 9:138022367-138023579 | |

### Table 3A—OAC/EAC

The DMRs may be those disclosed in Table 3 of GB2215319.1 and which are also available at: https://doi.org/10.5281/zenodo.10001260 as of 17 Oct. 2023. The entire contents of Table 3 of GB2215319.1 and https://doi.org/10.5281/zenodo.10001260 are expressly incorporated herein by reference as “Table 3A”.

TABLE 4
PrCa

| | | |
|---|---|---|
| 1:970457-971735 | 1:998042-998855 | 1:1033511-1034623 |
| 1:1246425-1247195 | 1:1351336-1351737 | 1:1630400-1631631 |
| 1:1670780-1671501 | 1:2003458-2004272 | 1:2081917-2083339 |
| 1:2127200-2127797 | 1:2174582-2174983 | 1:2290301-2291223 |
| 1:2299014-2299527 | 1:2529171-2530640 | 1:2610948-2611349 |
| 1:2789255-2790437 | 1:2830338-2831467 | 1:2851827-2852971 |
| 1:2932779-2933267 | 1:3001973-3002374 | 1:3032878-3033277 |
| 1:3089614-3090341 | 1:3141436-3142107 | 1:3412281-3412878 |
| 1:3425164-3425787 | 1:3587410-3587924 | 1:3598150-3598780 |
| 1:3684020-3684465 | 1:3703398-3704477 | 1:3910308-3910709 |
| 1:4149037-4149615 | 1:4152378-4152769 | 1:4362616-4363015 |
| 1:4497055-4497577 | 1:6358801-6359858 | 1:6418575-6419514 |
| 1:6425046-6425525 | 1:6491092-6492608 | 1:6599043-6600081 |
| 1:6603453-6604358 | 1:7373947-7374454 | 1:7453933-7454518 |
| 1:8121135-8121840 | 1:8154743-8156012 | 1:8217183-8217836 |
| 1:9069277-9070187 | 1:9689852-9690458 | 1:9827602-9828197 |
| 1:10032010-10032409 | 1:11692408-11693629 | 1:12124300-12124700 |
| 1:12739466-12739967 | 1:13583867-13584266 | 1:15154720-15155195 |
| 1:15187460-15187989 | 1:15246058-15246531 | 1:15343662-15344295 |
| 1:15735978-15737127 | 1:15949434-15950098 | 1:16154094-16154549 |
| 1:16156129-16156560 | 1:16162386-16162786 | 1:16173101-16173573 |
| 1:16226807-16227207 | 1:16349248-16349797 | 1:16647239-16648666 |
| 1:16759164-16760112 | 1:16921270-16922273 | 1:16978396-16979042 |
| 1:17569529-17570337 | 1:18630375-18630888 | 1:18631439-18632023 |
| 1:18949591-18950010 | 1:18949768-18951173 | 1:19288435-19288959 |
| 1:19665380-19665824 | 1:20185813-20186213 | 1:20343148-20343632 |
| 1:20473155-20473709 | 1:20716253-20716731 | 1:21262348-21263172 |
| 1:21427926-21428556 | 1:21814320-21814721 | 1:22318337-22318949 |
| 1:22350049-22350667 | 1:23825938-23826434 | 1:24322000-24323327 |
| 1:24419285-24419770 | 1:24593602-24594779 | 1:24930732-24932206 |
| 1:25617872-25618461 | 1:25875799-25876260 | 1:26225068-26225775 |
| 1:26359895-26360296 | 1:26410626-26411027 | 1:27428120-27429035 |
| 1:29631302-29632471 | 1:30885427-30885959 | 1:30907377-30907778 |
| 1:31181357-31181756 | 1:31181794-31182312 | 1:31372903-31373304 |
| 1:31373701-31374176 | 1:31482615-31483795 | 1:31575536-31578169 |
| 1:31703816-31704417 | 1:31772171-31772774 | 1:31944459-31944962 |
| 1:31954640-31955200 | 1:32201426-32201901 | 1:32322587-32323134 |
| 1:32703414-32703895 | 1:32885819-32886218 | 1:33081825-33083206 |
| 1:33099268-33099970 | 1:33472439-33472856 | 1:34176537-34177384 |
| 1:34780056-34781327 | 1:34792842-34793328 | 1:35577206-35577607 |
| 1:35708203-35708604 | 1:37471434-37472156 | 1:37735097-37735601 |
| 1:37764159-37765477 | 1:38004606-38005086 | 1:38030020-38030457 |
| 1:38559408-38559807 | 1:39080799-39081200 | 1:39082049-39082933 |
| 1:39268433-39269042 | 1:39458445-39459114 | 1:39515509-39515908 |
| 1:39639256-39639782 | 1:39672137-39672536 | 1:39683480-39684431 |
| 1:39692390-39693037 | 1:39789187-39789618 | 1:39960348-39961089 |
| 1:40315082-40315607 | 1:40315466-40316095 | 1:40654087-40654531 |
| 1:40798562-40799950 | 1:40818738-40819138 | 1:40861027-40861627 |
| 1:41358364-41358895 | 1:41380848-41381248 | 1:41381410-41381809 |
| 1:41483941-41484285 | 1:42784869-42785732 | 1:43067974-43068434 |
| 1:43206720-43207249 | 1:43454225-43454625 | 1:44237927-44238626 |
| 1:44784052-44784744 | 1:44819998-44820540 | 1:45326719-45327510 |
| 1:46166873-46167349 | 1:46203398-46203824 | 1:46447964-46449006 |
| 1:46485495-46486067 | 1:46543884-46544583 | 1:46613799-46614200 |
| 1:47229315-47229799 | 1:47232125-47232604 | 1:47416840-47417240 |
| 1:47433281-47434065 | 1:47444633-47445321 | 1:47532955-47534440 |
| 1:48471193-48471924 | 1:50415041-50415697 | 1:50417190-50417872 |
| 1:50418419-50419022 | 1:50421086-50421487 | 1:50422767-50423988 |
| 1:50424817-50425217 | 1:50427646-50428136 | 1:50430376-50430776 |
| 1:52601849-52603205 | 1:52842945-52843739 | 1:53061589-53062054 |
| 1:53444646-53445474 | 1:53737854-53738581 | 1:54474709-54475508 |
| 1:54542495-54542993 | 1:54547885-54548433 | 1:54800456-54800945 |
| 1:56376517-56377050 | 1:56577328-56578136 | 1:58248631-58251011 |
| 1:59146585-59147188 | 1:59673468-59674929 | 1:60073408-60074153 |
| 1:61053650-61054368 | 1:62194717-62195118 | 1:62318429-62318829 |
| 1:63319499-63320426 | 1:63326873-63327342 | 1:63329702-63330904 |
| 1:64471511-64471995 | 1:65002183-65002927 | 1:65002948-65003388 |
| 1:65264505-65264961 | 1:65265273-65266205 | 1:65525881-65526282 |
| 1:65791988-65792513 | 1:66532813-66534280 | 1:66751711-66752760 |
| 1:67134713-67135302 | 1:67749585-67750436 | 1:69569246-69570573 |
| 1:75130084-75130485 | 1:75136331-75136878 | 1:75616457-75617567 |
| 1:76074602-76075106 | 1:76868810-76869366 | 1:78045765-78046445 |
| 1:79006369-79007024 | 1:84998166-84998728 | 1:86755455-86755869 |
| 1:87151876-87152439 | 1:89843700-89844196 | 1:90717019-90717420 |
| 1:90717989-90718883 | 1:90719062-90720515 | 1:90723459-90723908 |
| 1:90724297-90724767 | 1:90726701-90727101 | 1:90727043-90727443 |
| 1:90728966-90729929 | 1:90834370-90836564 | 1:90850528-90850928 |
| 1:91830142-91830731 | 1:91891660-91892052 | 1:92480171-92482617 |
| 1:92486696-92487193 | 1:93680788-93681299 | 1:94927218-94928023 |
| 1:98052852-98053918 | 1:100537857-100539067 | 1:107139452-107140103 |
| 1:108041550-108042309 | 1:109649548-109650955 | 1:109656271-109656671 |
| 1:109667492-109668591 | 1:109687687-109688087 | 1:109791910-109792672 |
| 1:110068076-110068477 | 1:110068445-110069240 | 1:110129998-110130932 |
| 1:110606019-110606420 | 1:110675012-110676001 | 1:111271079-111271736 |
| 1:111515463-111516278 | 1:112509204-112509696 | 1:112722637-112723281 |
| 1:112743837-112744699 | 1:114154167-114154567 | 1:114854604-114855573 |
| 1:115062647-115063620 | 1:115828119-115829004 | 1:115838703-115839189 |
| 1:116168171-116168868 | 1:116878558-116879552 | 1:117759072-117759445 |
| 1:118984234-118984683 | 1:118984864-118985411 | 1:118985390-118986887 |
| 1:118987155-118987575 | 1:118987826-118988620 | 1:118989226-118989627 |
| 1:118989331-118989732 | 1:118993043-118993456 | 1:118998987-119002950 |
| 1:119005753-119008847 | 1:119327123-119328330 | 1:119713129-119713800 |
| 1:121006882-121011008 | 1:121048257-121048954 | 1:121117675-121118754 |
| 1:121396991-121397591 | 1:143574910-143575309 | 1:144456359-144456922 |
| 1:145459909-145460933 | 1:146019203-146019751 | 1:147072300-147073302 |
| 1:147077417-147081178 | 1:147083879-147085463 | 1:148048523-148049755 |
| 1:148280551-148280951 | 1:148317215-148318622 | 1:148361830-148363063 |
| 1:149747748-149748148 | 1:150150696-150151450 | 1:150282680-150283243 |
| 1:151131015-151132041 | 1:151145425-151146091 | 1:152515466-152515943 |
| 1:152698721-152699381 | 1:153310690-153311668 | 1:153349280-153350162 |
| 1:153416948-153418030 | 1:153428081-153428583 | 1:153457670-153458097 |
| 1:153526064-153526827 | 1:153536469-153536958 | 1:153565781-153567223 |
| 1:154502441-154502847 | 1:154969812-154970950 | 1:154999295-154999814 |
| 1:155062711-155063112 | 1:155294544-155296006 | 1:155320593-155321320 |
| 1:155976182-155976706 | 1:155978848-155979472 | 1:156106360-156106760 |
| 1:156125202-156127098 | 1:156160551-156161572 | 1:156245663-156246064 |
| 1:156388288-156388766 | 1:156420121-156420521 | 1:156420294-156420881 |
| 1:156435445-156435846 | 1:156436406-156437069 | 1:156676350-156676993 |
| 1:156706591-156707193 | 1:156844698-156846189 | 1:157768463-157768864 |
| 1:157978644-157979376 | 1:158330792-158331811 | 1:158352902-158354832 |
| 1:158420708-158421119 | 1:158718799-158719287 | 1:158753998-158755154 |
| 1:159008527-159009317 | 1:159088441-159089429 | 1:159314141-159314658 |
| 1:159713678-159715045 | 1:160285950-160286705 | 1:160399864-160400264 |
| 1:160707852-160709473 | 1:160981734-160982486 | 1:161481224-161482275 |
| 1:161727300-161727934 | 1:162003667-162004334 | 1:162822151-162822606 |
| 1:164651569-164652033 | 1:165352353-165353012 | 1:165355372-165355773 |
| 1:166884190-166884702 | 1:167121230-167121670 | 1:169427151-169427892 |
| 1:169460061-169460966 | 1:170661392-170661793 | 1:170663985-170665187 |
| 1:170730055-170731725 | 1:171670920-171671397 | 1:171842008-171842539 |
| 1:173122872-173123816 | 1:173207072-173207512 | 1:173669400-173670146 |
| 1:175599092-175599899 | 1:179575359-179576816 | 1:179744341-179745080 |
| 1:180153813-180154510 | 1:180235688-180236172 | 1:180913000-180913806 |
| 1:181099456-181100104 | 1:181728324-181728963 | 1:182056758-182057450 |
| 1:182794866-182795966 | 1:183842493-183842947 | 1:186680528-186680928 |
| 1:186681128-186681527 | 1:192904581-192904982 | 1:197775602-197776259 |
| 1:197910435-197910836 | 1:197912889-197913489 | 1:197918368-197918975 |
| 1:197919188-197919900 | 1:198938237-198938683 | 1:199007443-199009726 |
| 1:200034063-200036855 | 1:200038697-200039098 | 1:200040551-200042603 |
| 1:200042405-200042880 | 1:200302833-200303354 | 1:200873290-200874099 |
| 1:201259877-201260484 | 1:201283455-201283970 | 1:201531053-201531618 |
| 1:201539873-201540522 | 1:203199955-203200412 | 1:203627661-203628107 |
| 1:203629229-203630177 | 1:204151091-204151599 | 1:204151300-204151699 |
| 1:204359564-204360330 | 1:205313670-205314451 | 1:205524963-205525496 |
| 1:205568526-205568927 | 1:205662732-205663311 | 1:206506766-206507276 |
| 1:206681301-206682605 | 1:206686318-206687032 | 1:207645272-207646002 |
| 1:207818101-207818534 | 1:207958892-207959592 | 1:209397455-209399245 |
| 1:209627855-209628284 | 1:209769232-209769937 | 1:210327875-210328509 |
| 1:211829277-211829969 | 1:212671271-212672120 | 1:212916623-212917048 |
| 1:214658716-214659199 | 1:217137259-217138526 | 1:218156550-218156951 |
| 1:220876908-220878951 | 1:220880872-220882644 | 1:220887147-220888062 |
| 1:220890895-220892130 | 1:220894144-220896084 | 1:223007205-223007823 |
| 1:223364841-223365435 | 1:224456813-224457209 | 1:224498156-224499104 |
| 1:224929133-224929533 | 1:226034521-226034990 | 1:226661885-226663164 |
| 1:227559452-227561437 | 1:228446052-228446714 | 1:228464602-228465001 |
| 1:228469478-228470242 | 1:229406895-229408885 | 1:229431314-229433896 |
| 1:229841184-229841657 | 1:230507925-230508440 | 1:231041088-231041605 |
| 1:231160921-231161507 | 1:234451732-234452172 | 1:234613457-234613982 |
| 1:234656907-234658466 | 1:234772191-234772927 | 1:234969853-234970275 |
| 1:234970004-234971568 | 1:235650558-235650984 | 1:235917067-235918146 |
| 1:236065242-236065642 | 1:236065345-236065816 | 1:236143033-236143801 |
| 1:238274696-238276221 | 1:240093152-240094035 | 1:241306269-241306728 |
| 1:242604274-242604790 | 1:243916905-243917305 | 1:244341720-244342270 |
| 1:244729948-244730401 | 1:244801192-244801617 | 1:246775735-246776400 |
| 1:246782514-246783067 | 1:246788224-246789446 | 1:247332600-247333423 |
| 1:247406885-247407447 | 1:247516183-247517049 | 10:436296-437691 |
| 10:1411944-1412344 | 10:1463191-1463686 | 10:3066164-3067169 |
| 10:3095586-3096369 | 10:3287422-3287924 | 10:3288133-3288533 |
| 10:3668484-3669063 | 10:5646266-5646845 | 10:5692457-5692903 |
| 10:6489786-6490196 | 10:7667623-7668366 | 10:8054877-8055541 |
| 10:8055440-8059754 | 10:9016712-9017349 | 10:11164644-11165188 |
| 10:11792530-11793348 | 10:12631407-12632316 | 10:13001162-13001571 |
| 10:13349909-13350309 | 10:13529497-13529976 | 10:14836964-14837437 |
| 10:15439497-15439975 | 10:16519391-16520621 | 10:17227625-17228245 |
| 10:17228100-17228582 | 10:21525116-21525621 | 10:22251143-22251517 |
| 10:22335294-22336068 | 10:23172094-23174079 | 10:23174163-23174598 |
| 10:24951268-24952378 | 10:25933659-25935305 | 10:26586273-26586678 |
| 10:26636644-26638255 | 10:26869450-26869918 | 10:26931154-26931662 |
| 10:27259122-27259823 | 10:27319623-27320656 | 10:27746551-27747372 |
| 10:29659244-29659649 | 10:30098690-30100035 | 10:30219847-30220476 |
| 10:30434940-30435595 | 10:31137283-31137780 | 10:42474973-42475373 |
| 10:42898129-42898548 | 10:42932872-42934340 | 10:43104550-43105408 |
| 10:43202156-43202928 | 10:43667182-43667972 | 10:45418677-45419652 |
| 10:46063037-46063778 | 10:46465760-46466167 | 10:46465879-46466279 |
| 10:46555324-46556557 | 10:48658521-48660173 | 10:48697201-48697614 |
| 10:48982597-48984162 | 10:49133724-49134453 | 10:49396262-49396662 |
| 10:49608766-49609208 | 10:49768053-49769234 | 10:50417842-50418795 |
| 10:52771434-52771850 | 10:55627591-55629049 | 10:58512069-58512524 |
| 10:58967550-58968095 | 10:61869574-61869974 | 10:66928042-66928559 |
| 10:68561335-68563488 | 10:69087433-69087834 | 10:70132103-70132584 |
| 10:70710642-70712657 | 10:71395859-71396844 | 10:71597038-71598288 |
| 10:71729082-71729781 | 10:71860232-71860765 | 10:72086949-72087924 |
| 10:72088244-72089218 | 10:72260521-72261881 | 10:73647725-73648383 |
| 10:73889200-73889530 | 10:73910393-73910917 | 10:74589276-74589870 |
| 10:75409782-75410201 | 10:75430145-75430829 | 10:77531692-77532448 |
| 10:77636151-77637185 | 10:77710911-77711358 | 10:77872436-77873965 |
| 10:78756891-78757331 | 10:79242049-79244050 | 10:79248378-79249021 |
| 10:84256986-84257386 | 10:86047532-86048146 | 10:86367495-86368024 |
| 10:86968809-86972182 | 10:88979054-88979543 | 10:89535374-89535774 |
| 10:89535735-89536248 | 10:89837984-89838597 | 10:91744899-91745403 |
| 10:91887023-91888178 | 10:92419765-92421147 | 10:92751018-92751443 |
| 10:92823035-92823628 | 10:93060027-93063584 | 10:93065639-93066712 |
| 10:94088743-94089421 | 10:95406984-95407443 | 10:95467203-95467723 |
| 10:95501648-95502183 | 10:97185117-97185524 | 10:97713177-97713617 |
| 10:97714613-97715847 | 10:97771798-97772804 | 10:97788658-97789193 |
| 10:98446136-98446537 | 10:99233613-99234012 | 10:99234606-99237199 |
| 10:99519789-99524038 | 10:99526632-99528363 | 10:99530052-99531682 |
| 10:99535816-99539206 | 10:100115074-100115474 | 10:100636047-100636819 |
| 10:100659301-100660010 | 10:100687463-100688588 | 10:100715416-100715822 |
| 10:100735233-100735902 | 10:100737368-100737769 | 10:100830173-100830837 |
| 10:100988998-100989593 | 10:101000795-101001264 | 10:101032135-101032974 |
| 10:101047941-101048645 | 10:101131022-101131423 | 10:101132223-101133085 |
| 10:101134017-101134541 | 10:101134731-101135526 | 10:101135800-101137261 |
| 10:101139316-101139717 | 10:101140041-101140884 | 10:101145679-101146565 |
| 10:101225806-101226699 | 10:101227065-101227651 | 10:101236289-101236857 |
| 10:101237919-101238320 | 10:101284083-101284587 | 10:101310464-101310896 |
| 10:101776388-101776787 | 10:101829230-101830381 | 10:102121071-102121873 |
| 10:102674052-102674453 | 10:102676232-102676911 | 10:102869759-102870262 |
| 10:103276703-103277121 | 10:103652714-103654089 | 10:103659635-103662101 |
| 10:103693268-103693668 | 10:103779395-103779886 | 10:104253906-104254451 |
| 10:104322337-104322978 | 10:104328044-104328538 | 10:104328770-104329170 |
| 10:106183245-106184591 | 10:106629279-106629732 | 10:107163886-107165097 |
| 10:110005960-110007772 | 10:110221698-110222396 | 10:110392872-110393314 |
| 10:110850942-110851454 | 10:111078309-111079552 | 10:114178819-114180108 |
| 10:114404939-114405581 | 10:114648358-114648909 | 10:116669771-116670810 |
| 10:117131315-117131905 | 10:117131873-117135259 | 10:117139578-117139978 |
| 10:117240553-117241277 | 10:117541802-117542399 | 10:117544825-117545225 |
| 10:117545276-117545956 | 10:117553104-117553714 | 10:117734832-117735310 |
| 10:118593994-118596831 | 10:118669337-118669844 | 10:118832233-118832813 |
| 10:121595603-121597151 | 10:122163038-122164085 | 10:123137046-123137787 |
| 10:123665788-123666984 | 10:123761830-123762229 | 10:123857530-123857962 |
| 10:123858521-123859179 | 10:124036181-124036678 | 10:124340941-124341404 |
| 10:124447069-124447553 | 10:124640854-124641909 | 10:124648425-124649088 |
| 10:126388597-126389494 | 10:126906048-126906823 | 10:127195345-127195746 |
| 10:127735681-127739397 | 10:129715800-129716292 | 10:129949972-129950390 |
| 10:131812108-131812628 | 10:131982684-131983896 | 10:131993181-131993580 |
| 10:132190942-132192246 | 10:132544880-132545331 | 10:132851451-132851851 |
| 10:132916368-132917203 | 10:133083364-133083835 | 10:133100180-133101345 |
| 10:133115139-133115827 | 10:133203039-133203738 | 10:133216829-133217229 |
| 10:133309753-133310410 | 10:133376659-133377506 | 11:288102-288503 |
| 11:313378-314643 | 11:319404-320679 | 11:504150-505087 |
| 11:561941-562697 | 11:569180-569695 | 11:626963-627363 |
| 11:710966-711367 | 11:823980-825064 | 11:829632-830677 |
| 11:843726-844126 | 11:979875-980802 | 11:1028975-1029374 |
| 11:1337134-1337595 | 11:1388446-1389276 | 11:1747692-1748459 |
| 11:1836538-1837055 | 11:1916035-1916435 | 11:2271097-2271498 |
| 11:2796625-2797994 | 11:2825999-2826873 | 11:2869284-2869685 |
| 11:2883082-2884292 | 11:3159203-3161198 | 11:4058125-4058526 |
| 11:4608288-4608913 | 11:5302746-5303297 | 11:5324776-5325175 |
| 11:5343295-5344297 | 11:5509810-5510211 | 11:5993022-5994774 |
| 11:6418405-6418804 | 11:6845248-6845660 | 11:6926335-6926734 |
| 11:7674075-7674475 | 11:7963485-7964166 | 11:9091684-9092085 |
| 11:10294761-10295160 | 11:11156254-11157644 | 11:12009325-12009725 |
| 11:12680201-12680602 | 11:13008502-13009015 | 11:13010865-13011266 |
| 11:14905012-14905412 | 11:15114244-15114793 | 11:17405299-17405868 |
| 11:17695861-17696261 | 11:18208924-18209485 | 11:19241958-19242877 |
| 11:19714437-19715698 | 11:20160152-20160553 | 11:20596477-20596878 |
| 11:20670857-20671548 | 11:22626344-22626744 | 11:26993958-26994594 |
| 11:28566442-28567112 | 11:30016107-30016506 | 11:30583713-30584390 |
| 11:31802546-31802946 | 11:31804046-31804446 | 11:31804722-31805176 |
| 11:31805335-31805736 | 11:31817929-31818549 | 11:31819826-31820371 |
| 11:31821690-31822489 | 11:31823691-31824242 | 11:31825087-31825486 |
| 11:31826790-31827252 | 11:31987236-31987636 | 11:32333045-32333626 |
| 11:32426596-32428425 | 11:32428407-32429296 | 11:32430895-32431443 |
| 11:32433278-32433966 | 11:32436939-32437338 | 11:32437360-32438558 |
| 11:32439479-32439880 | 11:32893800-32894724 | 11:33016095-33016917 |
| 11:33870495-33870894 | 11:34600281-34601201 | 11:35138632-35139065 |
| 11:35420079-35420503 | 11:35525783-35526182 | 11:35617577-35618288 |
| 11:36375757-36376332 | 11:36377574-36378119 | 11:43581132-43581532 |
| 11:43649491-43650064 | 11:44303053-44306176 | 11:44311189-44311588 |
| 11:44319660-44320246 | 11:44569138-44569539 | 11:45073618-45074120 |
| 11:45080156-45081047 | 11:45091744-45092250 | 11:45145024-45146108 |
| 11:46295226-46296283 | 11:46344718-46346447 | 11:46361026-46361950 |
| 11:46392947-46393287 | 11:46918734-46919385 | 11:47186835-47187911 |
| 11:47257275-47258186 | 11:47400589-47401063 | 11:47589472-47590558 |
| 11:47714272-47714753 | 11:47885976-47886564 | 11:47917745-47918475 |
| 11:48019071-48019796 | 11:49558348-49558753 | 11:56275413-56276188 |
| 11:57180129-57180694 | 11:57425792-57427239 | 11:57482206-57483525 |
| 11:57598101-57598626 | 11:57637654-57638240 | 11:58844371-58845692 |
| 11:59173209-59174092 | 11:59216811-59217250 | 11:59443010-59444108 |
| 11:60699697-60701508 | 11:60821479-60822189 | 11:60855793-60856542 |
| 11:61295261-61295660 | 11:61516345-61517095 | 11:61567589-61567989 |
| 11:61827308-61827709 | 11:61828333-61829517 | 11:62269570-62270313 |
| 11:62440280-62440683 | 11:62443989-62444975 | 11:62602589-62603362 |
| 11:62923443-62924130 | 11:62925873-62928496 | 11:62999548-62999985 |
| 11:63128071-63128650 | 11:63490886-63491457 | 11:63680708-63681150 |
| 11:63769509-63770257 | 11:63998369-63999191 | 11:64060324-64061605 |
| 11:64292254-64292654 | 11:64553223-64553663 | 11:64711390-64711791 |
| 11:65047466-65048386 | 11:65108054-65108951 | 11:65557146-65557928 |
| 11:65832959-65833591 | 11:65871959-65872816 | 11:65872739-65873140 |
| 11:65898890-65899437 | 11:66316847-66317339 | 11:66336473-66336873 |
| 11:66559145-66559679 | 11:67371801-67372225 | 11:67442506-67442906 |
| 11:67583354-67584841 | 11:67665414-67666002 | 11:68328004-68328847 |
| 11:68632761-68633328 | 11:69443284-69443746 | 11:69572920-69573459 |
| 11:69636493-69637027 | 11:69637429-69637830 | 11:69653389-69653788 |
| 11:69702184-69702853 | 11:70124207-70124776 | 11:70601889-70603333 |
| 11:70628761-70629232 | 11:70661924-70662705 | 11:70826255-70827301 |
| 11:71240928-71241329 | 11:72244137-72244705 | 11:74466918-74467900 |
| 11:74696962-74697379 | 11:75583907-75584464 | 11:76321859-76322497 |
| 11:76860066-76860544 | 11:76860251-76860652 | 11:77147564-77148720 |
| 11:77472611-77473300 | 11:78349330-78350701 | 11:82732361-82732760 |
| 11:82750701-82751275 | 11:84719726-84720403 | 11:86374501-86375040 |
| 11:88115949-88116501 | 11:88326208-88326855 | 11:89490662-89491271 |
| 11:89491186-89491667 | 11:89491399-89491799 | 11:90134252-90134928 |
| 11:90422569-90423031 | 11:92224343-92225380 | 11:92942309-92943744 |
| 11:93329891-93330500 | 11:93537818-93538390 | 11:96338730-96339505 |
| 11:96341579-96341980 | 11:101582659-101584103 | 11:105609853-105610253 |
| 11:105610101-105610729 | 11:111298252-111299290 | 11:111299829-11130056 |
| 11:111512633-111513034 | 11:111513013-111513473 | 11:111514530-111514930 |
| 11:111540004-111541181 | 11:111912386-111912787 | 11:111976600-111977173 |
| 11:112120140-112120938 | 11:113387339-113387965 | 11:115659502-115660022 |
| 11:116500320-116501355 | 11:116580170-116581163 | 11:117877068-117877922 |
| 11:118141850-118143108 | 11:118621547-118621948 | 11:118909720-118915026 |
| 11:119422954-119423355 | 11:119742200-119743297 | 11:120209855-120210345 |
| 11:120239593-120240049 | 11:120564020-120564497 | 11:122152140-122152946 |
| 11:122180771-122181286 | 11:123118533-123118868 | 11:123806032-123806433 |
| 11:123943762-123944645 | 11:124014837-124015584 | 11:124028797-124029318 |
| 11:124897970-124898949 | 11:125262946-125263863 | 11:125496451-125496852 |
| 11:125874030-125874431 | 11:125904331-125904731 | 11:126318653-126319347 |
| 11:128549153-128549611 | 11:129373173-129373711 | 11:131422454-131423938 |
| 11:132943517-132944074 | 11:133076646-133077189 | 11:133228366-133228766 |
| 11:133917966-133919228 | 11:134036631-134037032 | 12:460789-461346 |
| 12:546512-546948 | 12:1661382-1661782 | 12:1936678-1937079 |
| 12:2054203-2054604 | 12:2834846-2835925 | 12:3055506-3055922 |
| 12:3341912-3342489 | 12:3454174-3454914 | 12:3752904-3753559 |
| 12:4268925-4269325 | 12:4272075-4272475 | 12:4272429-4272920 |
| 12:4275518-4275918 | 12:5433078-5433720 | 12:6199441-6200009 |
| 12:6376532-6376932 | 12:6553169-6556384 | 12:6829320-6830035 |
| 12:6967942-6969124 | 12:7068525-7068906 | 12:7091615-7092408 |
| 12:7502635-7504095 | 12:7860280-7860785 | 12:7872791-7873191 |
| 12:7971295-7972153 | 12:8365002-8365822 | 12:8680519-8680919 |
| 12:9649430-9651201 | 12:9947818-9948829 | 12:11652057-11653833 |
| 12:11812895-11815032 | 12:12010382-12010847 | 12:14238466-14238930 |
| 12:14567590-14568505 | 12:15984119-15985318 | 12:16604866-16605266 |
| 12:21940874-21941275 | 12:22333732-22334675 | 12:24663852-24664630 |
| 12:24902881-24903459 | 12:25384954-25385465 | 12:27017303-27017882 |
| 12:27462054-27462606 | 12:27549119-27550096 | 12:29149431-29150437 |
| 12:29610310-29610903 | 12:29782327-29783090 | 12:30169757-30170772 |
| 12:30753638-30754203 | 12:31682195-31682637 | 12:34107593-34108795 |
| 12:34355266-34356611 | 12:34398908-34399626 | 12:34602482-34603257 |
| 12:40168128-40169255 | 12:40692876-40693277 | 12:43550971-43552419 |
| 12:45049786-45050787 | 12:45234302-45235957 | 12:46830715-46831321 |
| 12:46831961-46832780 | 12:47812832-47813232 | 12:48003796-48004239 |
| 12:48197739-48198832 | 12:49090930-49091627 | 12:49295076-49296409 |
| 12:49827075-49827926 | 12:49903543-49904312 | 12:49960947-49962081 |
| 12:49983747-49984270 | 12:50050588-50051090 | 12:50222060-50224017 |
| 12:51387993-51388470 | 12:51390807-51391281 | 12:51863958-51864630 |
| 12:52006715-52007889 | 12:52203414-52203809 | 12:52232129-52234089 |
| 12:52244070-52245718 | 12:52713925-52714326 | 12:52730801-52732293 |
| 12:52789838-52790452 | 12:52867497-52868008 | 12:52873691-52874799 |
| 12:52965220-52965872 | 12:53218111-53220038 | 12:53223397-53223798 |
| 12:53325634-53326143 | 12:53676956-53677560 | 12:53695360-53695863 |
| 12:53696529-53697120 | 12:53739450-53739850 | 12:53751856-53752257 |
| 12:53927411-53927868 | 12:53961039-53961453 | 12:53965826-53966497 |
| 12:53972825-53973275 | 12:53994162-53994563 | 12:53995244-53996257 |
| 12:54006543-54007161 | 12:54014156-54016137 | 12:54019058-54020303 |
| 12:54026849-54027400 | 12:54029493-54029932 | 12:54030150-54031784 |
| 12:54034236-54034958 | 12:54046095-54047875 | 12:54050966-54051464 |
| 12:54053009-54055319 | 12:54106130-54107397 | 12:54126934-54127335 |
| 12:54217836-54218498 | 12:54259130-54259793 | 12:55414087-55415263 |
| 12:55707343-55707744 | 12:55830865-55831537 | 12:56085281-56088028 |
| 12:56256287-56257545 | 12:57226673-57227223 | 12:57236260-57238487 |
| 12:57627779-57628284 | 12:57893872-57894659 | 12:62191099-62191573 |
| 12:62631282-62633178 | 12:62813178-62813578 | 12:64782472-64783049 |
| 12:64824138-64826664 | 12:65121489-65121890 | 12:65278100-65278544 |
| 12:65728943-65729497 | 12:66234820-66235469 | 12:68364909-68365790 |
| 12:75334173-75334582 | 12:75334484-75334884 | 12:76029975-76030376 |
| 12:76877999-76878495 | 12:77325387-77325787 | 12:80708105-80709121 |
| 12:80714005-80714586 | 12:80715704-80717317 | 12:81077381-81078548 |
| 12:84912986-84913523 | 12:85273424-85273988 | 12:85279292-85279719 |
| 12:89349860-89351250 | 12:89354696-89355751 | 12:89370712-89371184 |
| 12:89707395-89707936 | 12:93021072-93021625 | 12:93894576-93894977 |
| 12:95547815-95548601 | 12:96195998-96197043 | 12:97148942-97149428 |
| 12:98893200-98893930 | 12:99600198-99600741 | 12:100356544-100357423 |
| 12:100715856-100716933 | 12:101642206-101642812 | 12:101935997-101936553 |
| 12:102917159-102917968 | 12:102955984-102956483 | 12:102961167-102961701 |
| 12:102962029-102962432 | 12:102964682-102965081 | 12:102964952-102965645 |
| 12:102965516-102966208 | 12:103302145-103302790 | 12:103571414-103571916 |
| 12:104177999-104178598 | 12:104215471-104216379 | 12:104303458-104303886 |
| 12:104303597-104303998 | 12:104458118-104458921 | 12:104720233-104720699 |
| 12:106139587-106140813 | 12:106248958-106249359 | 12:106585133-106585854 |
| 12:106954921-106955321 | 12:107774043-107776098 | 12:107844009-107844835 |
| 12:108638333-108638979 | 12:108838271-108838742 | 12:109115826-109116648 |
| 12:109291520-109291920 | 12:109712094-109712606 | 12:109734252-109734691 |
| 12:109832607-109833108 | 12:110266955-110267521 | 12:110585242-110585785 |
| 12:110684356-110684848 | 12:110689183-110689878 | 12:111180870-111181801 |
| 12:111767190-111767714 | 12:113142037-113143054 | 12:113149074-113150036 |
| 12:113478517-113479009 | 12:114399585-114400811 | 12:114685801-114688560 |
| 12:114691055-114692146 | 12:114693267-114695234 | 12:114697903-114698653 |
| 12:115598922-115599639 | 12:116318994-116320391 | 12:116443751-116444788 |
| 12:116559067-116559880 | 12:116598716-116599116 | 12:116598945-116599345 |
| 12:116693567-116694064 | 12:120361378-120362259 | 12:120388957-120389426 |
| 12:120649861-120650635 | 12:121336692-121337229 | 12:121578162-121579297 |
| 12:121580938-121581337 | 12:122423106-122423940 | 12:122646899-122647420 |
| 12:122946451-122947613 | 12:123432415-123433318 | 12:123519143-123519574 |
| 12:123761693-123762826 | 12:124296112-124296642 | 12:124379685-124380784 |
| 12:124548800-124549887 | 12:124626181-124626743 | 12:124686972-124688379 |
| 12:124727084-124727701 | 12:124754986-124755613 | 12:125974097-125975154 |
| 12:126183006-126183567 | 12:126741176-126742030 | 12:129902437-129902895 |
| 12:129926492-129926893 | 12:130160727-130161171 | 12:130162847-130164752 |
| 12:130657952-130658571 | 12:130917653-130918169 | 12:131139713-131140114 |
| 12:131347202-131347603 | 12:131431485-131431922 | 12:132170046-132170589 |
| 12:132489137-132489537 | 12:132689160-132690334 | 12:132777110-132777974 |
| 12:132804627-132805028 | 13:20142344-20142743 | 13:20161242-20161882 |
| 13:22926498-22926899 | 13:23903320-23903881 | 13:24328016-24328753 |
| 13:24982046-24982544 | 13:25114496-25114958 | 13:26785311-26785705 |
| 13:27790436-27790885 | 13:27792957-27794100 | 13:27924618-27925019 |
| 13:27927801-27928474 | 13:27967135-27967535 | 13:27970304-27970773 |
| 13:27975286-27976202 | 13:29902273-29902799 | 13:30576414-30576894 |
| 13:30833147-30834606 | 13:31209355-31210102 | 13:32428482-32428883 |
| 13:33205792-33208021 | 13:35470572-35471365 | 13:35475474-35479132 |
| 13:36154581-36155316 | 13:36297820-36298219 | 13:36345056-36346020 |
| 13:36430393-36430794 | 13:36431977-36432624 | 13:42573927-42574450 |
| 13:42574756-42575248 | 13:42991614-42992647 | 13:43055144-43055657 |
| 13:44373087-44374427 | 13:45615525-45615925 | 13:45851535-45851935 |
| 13:50527327-50528075 | 13:50843182-50843860 | 13:51335296-51335998 |
| 13:52738680-52739544 | 13:52845271-52845671 | 13:57633524-57633925 |
| 13:60473598-60474047 | 13:70107427-70109099 | 13:74132857-74133413 |
| 13:76887016-76887416 | 13:78595860-78596318 | 13:78607002-78607403 |
| 13:78607759-78608160 | 13:83881948-83882347 | 13:87674296-87674697 |
| 13:94968469-94968893 | 13:95002767-95003475 | 13:95337066-95337504 |
| 13:95552264-95552752 | 13:95552465-95553191 | 13:95643254-95644193 |
| 13:96993904-96995146 | 13:98142065-98142489 | 13:98577888-98578406 |
| 13:98751874-98752402 | 13:99614387-99615040 | 13:99894787-99895624 |
| 13:99895380-99895974 | 13:99955808-99956209 | 13:99958564-99959311 |
| 13:99971715-99972114 | 13:99974936-99975852 | 13:99984708-99985456 |
| 13:99988912-99989796 | 13:99990370-99990867 | 13:99990907-99992113 |
| 13:99992068-99992553 | 13:101916335-101916736 | 13:102367435-102368069 |
| 13:108213893-108214956 | 13:108269841-108270306 | 13:108496512-108496973 |
| 13:109140395-109141567 | 13:109642416-109642964 | 13:110280449-110281010 |
| 13:110362925-110363434 | 13:110520459-110521463 | 13:111683278-111683830 |
| 13:112053247-112058527 | 13:112062742-112063320 | 13:112067324-112067786 |
| 13:112104890-112105555 | 13:112745926-112746590 | 13:112940235-112940634 |
| 13:113325495-113326075 | 13:113364400-113365103 | 13:113437250-113437966 |
| 13:113454954-113455784 | 13:113491490-113491890 | 13:113647776-113648497 |
| 13:113862549-113863500 | 13:114031690-114032089 | 13:114041555-114042444 |
| 13:114260150-114260687 | 14:21023107-21024466 | 14:21025761-21026404 |
| 14:21071402-21072320 | 14:21230551-21231813 | 14:21526789-21527273 |
| 14:21536596-21537385 | 14:22836188-22836721 | 14:22838386-22839991 |
| 14:22979789-22980635 | 14:23351789-23352386 | 14:23365448-23367079 |
| 14:23694596-23695086 | 14:23988466-23989693 | 14:24036141-24036541 |
| 14:24171412-24173188 | 14:24264277-24264732 | 14:24334322-24335370 |
| 14:24339378-24340260 | 14:24368535-24369070 | 14:24397807-24399026 |
| 14:26204777-26205176 | 14:28765766-28766320 | 14:28767119-28767842 |
| 14:28774825-28775225 | 14:28777741-28778142 | 14:28784275-28786183 |
| 14:30875087-30876144 | 14:34713438-34713839 | 14:36503848-36504636 |
| 14:36505444-36505913 | 14:36514162-36514682 | 14:36516549-36517308 |
| 14:36517640-36518353 | 14:36521074-36522046 | 14:36522645-36523178 |
| 14:36524160-36524776 | 14:36663309-36664113 | 14:38209794-38211981 |
| 14:38254812-38256418 | 14:41605149-41605582 | 14:41606052-41606501 |
| 14:45095201-45097047 | 14:47674203-47676536 | 14:50060816-50061499 |
| 14:51093178-51094180 | 14:51095657-51096364 | 14:51328122-51329156 |
| 14:51507929-51508330 | 14:51508272-51508832 | 14:52069044-52069445 |
| 14:52069278-52069868 | 14:52789881-52790393 | 14:53955906-53956357 |
| 14:55071912-55072895 | 14:55107645-55108219 | 14:55128797-55129788 |
| 14:55454539-55455161 | 14:56412367-56412782 | 14:56794833-56795609 |
| 14:56797226-56797719 | 14:56798050-56798992 | 14:56809188-56809689 |
| 14:56810358-56811171 | 14:56811215-56812547 | 14:59919069-59920550 |
| 14:60506571-60506971 | 14:60506954-60507471 | 14:60507594-60507993 |
| 14:60637647-60638048 | 14:60651882-60652650 | 14:60653265-60653666 |
| 14:61525208-61525921 | 14:62116926-62117351 | 14:63852342-63852741 |
| 14:64708358-64709173 | 14:68316446-68316856 | 14:68779893-68781592 |
| 14:68814617-68816513 | 14:69711664-69712345 | 14:69768649-69769050 |
| 14:69769373-69770147 | 14:70186748-70187723 | 14:70821332-70821885 |
| 14:72521164-72522420 | 14:72700002-72700970 | 14:73741309-73741864 |
| 14:74239112-74239513 | 14:74239292-74239693 | 14:74571597-74572287 |
| 14:75279916-75280395 | 14:75427282-75427730 | 14:76929885-76930344 |
| 14:76955812-76956335 | 14:76968431-76968854 | 14:77043118-77044026 |
| 14:77158046-77158507 | 14:79280133-79280587 | 14:85529813-85530214 |
| 14:85529952-85532475 | 14:85622106-85622530 | 14:89503517-89504054 |
| 14:91113364-91114419 | 14:91946969-91947512 | 14:91947805-91948231 |
| 14:92513203-92513832 | 14:93787854-93788530 | 14:93939134-93939535 |
| 14:94768170-94769302 | 14:94772190-94772591 | 14:94772893-94773956 |
| 14:94773934-94774335 | 14:95320175-95320614 | 14:95514991-95515503 |
| 14:96177888-96178396 | 14:96592142-96593452 | 14:97119177-97120888 |
| 14:97777452-97777978 | 14:99229644-99230800 | 14:99659870-99660498 |
| 14:100297553-100298052 | 14:100567151-100567552 | 14:100851078-100852597 |
| 14:100949640-100950371 | 14:100966399-100967414 | 14:100968766-100969278 |
| 14:101063367-101064350 | 14:101072707-101073302 | 14:101853610-101854748 |
| 14:103058363-103058762 | 14:103071544-103071956 | 14:103084258-103084659 |
| 14:103138795-103139195 | 14:103189206-103189605 | 14:103273537-103273938 |
| 14:103705207-103706037 | 14:104085544-104086022 | 14:104107062-104107579 |
| 14:104117310-104118309 | 14:104289814-104290346 | 14:104588388-104588994 |
| 14:104801903-104802596 | 14:104863664-104864349 | 14:105221641-105222225 |
| 14:105527261-105527880 | 15:20356447-20356973 | 15:22714287-22714908 |
| 15:22933501-22934203 | 15:25780814-25781215 | 15:25802071-25802526 |
| 15:26771382-26771822 | 15:26867970-26868770 | 15:27541698-27542370 |
| 15:28096486-28097552 | 15:28788596-28789167 | 15:29103528-29103929 |
| 15:29115438-29116092 | 15:29675171-29675571 | 15:31392482-31393538 |
| 15:32716897-32717570 | 15:32802439-32802911 | 15:33194768-33195238 |
| 15:34721849-34722248 | 15:36468905-36469784 | 15:37094952-37098329 |
| 15:37102488-37103120 | 15:40283286-40284114 | 15:40439277-40439964 |
| 15:40768999-40769398 | 15:40769635-40770289 | 15:41512489-41514093 |
| 15:41881668-41882956 | 15:43517339-43517817 | 15:44193858-44194402 |
| 15:44830754-44831588 | 15:45114504-45114904 | 15:45116512-45117579 |
| 15:45129197-45129596 | 15:45129649-45130346 | 15:45187026-45187707 |
| 15:45378472-45378871 | 15:48645891-48646529 | 15:51093568-51094405 |
| 15:52783065-52783466 | 15:52791128-52791527 | 15:57528587-57529022 |
| 15:57565359-57565799 | 15:58864714-58865500 | 15:59437203-59437711 |
| 15:60002904-60003303 | 15:60396272-60397236 | 15:60398317-60398925 |
| 15:61584837-61585320 | 15:62164859-62165480 | 15:63042005-63042457 |
| 15:63380663-63381237 | 15:64503314-64504192 | 15:64700432-64701283 |
| 15:64776194-64776595 | 15:64893956-64894396 | 15:66621585-66622381 |
| 15:67120978-67121707 | 15:67826032-67826797 | 15:67827903-67829951 |
| 15:67967833-67968521 | 15:68431199-68431876 | 15:68795252-68795652 |
| 15:70023421-70024936 | 15:70266377-70267189 | 15:70348869-70349414 |
| 15:70377008-70377409 | 15:70585376-70585860 | 15:71116537-71117684 |
| 15:71526030-71526628 | 15:72196799-72197637 | 15:72271033-72273469 |
| 15:73318586-73319011 | 15:73369414-73369813 | 15:73750971-73751945 |
| 15:73785327-73785987 | 15:73925778-73926875 | 15:74132485-74135426 |
| 15:74365674-74366382 | 15:74588517-74588953 | 15:74726595-74727383 |
| 15:74958998-74959542 | 15:75178435-75179003 | 15:75693873-75694391 |
| 15:76335893-76336340 | 15:76337738-76338697 | 15:76994491-76995716 |
| 15:77496715-77497241 | 15:77818117-77818902 | 15:78988460-78988932 |
| 15:79089294-79089693 | 15:80749294-80749804 | 15:81133849-81134249 |
| 15:82555963-82557507 | 15:82648009-82648680 | 15:83282829-83283417 |
| 15:84657688-84658130 | 15:84817241-84817642 | 15:85619010-85619411 |
| 15:85752682-85753860 | 15:87123789-87124309 | 15:88258042-88258443 |
| 15:88635852-88636479 | 15:88894826-88895884 | 15:88912477-88913055 |
| 15:89360183-89360591 | 15:89377701-89378101 | 15:89378905-89379305 |
| 15:89406235-89406695 | 15:89408670-89409823 | 15:89409651-89410289 |
| 15:89496004-89497014 | 15:90211677-90213055 | 15:90829914-90830550 |
| 15:90883932-90884331 | 15:93028404-93028917 | 15:93071378-93072067 |
| 15:93073579-93074324 | 15:93089350-93089817 | 15:93589490-93589963 |
| 15:94265083-94266222 | 15:95326578-95327643 | 15:96367785-96368617 |
| 15:96437415-96438854 | 15:96440728-96442286 | 15:96447998-96448812 |
| 15:98101843-98103262 | 15:99732345-99732746 | 15:100201769-100202268 |
| 15:100372519-100373959 | 15:101127060-101127985 | 16:324566-325350 |
| 16:369712-370150 | 16:616972-617372 | 16:681366-682268 |
| 16:745676-746077 | 16:750276-751700 | 16:980293-980769 |
| 16:997638-998369 | 16:1066196-1066671 | 16:1198992-1199640 |
| 16:1334467-1334867 | 16:1435885-1439161 | 16:1680376-1680897 |
| 16:1688137-1688640 | 16:1962222-1963722 | 16:1990829-1992065 |
| 16:2054779-2055460 | 16:2715641-2716042 | 16:2835986-2836451 |
| 16:3017847-3018248 | 16:3029271-3030102 | 16:3047146-3047692 |
| 16:3161317-3161718 | 16:3171126-3171527 | 16:3187692-3189731 |
| 16:3789306-3789772 | 16:4428400-4428952 | 16:4474838-4475227 |
| 16:4537168-4538110 | 16:4943231-4944894 | 16:4987309-4987719 |
| 16:4987513-4988480 | 16:9013141-9013542 | 16:10179951-10183417 |
| 16:10818323-10818838 | 16:11640195-11641044 | 16:11669623-11670757 |
| 16:12147452-12147996 | 16:12903526-12904210 | 16:14501446-14502447 |
| 16:15989187-15989585 | 16:19114674-19115545 | 16:19118938-19119339 |
| 16:19882309-19884149 | 16:20690949-20691700 | 16:21283379-21283825 |
| 16:21511396-21512201 | 16:22814798-22815310 | 16:22947740-22948599 |
| 16:22948350-22948750 | 16:23497040-23497643 | 16:23754592-23754993 |
| 16:23835853-23836504 | 16:25692824-25694289 | 16:27230607-27232277 |
| 16:27315154-27316297 | 16:27318338-27319127 | 16:27449861-27450274 |
| 16:28596715-28597396 | 16:29074695-29075177 | 16:29149920-29150383 |
| 16:29160449-29161524 | 16:29678799-29679353 | 16:29691857-29692372 |
| 16:29809032-29809700 | 16:29876473-29876874 | 16:29877099-29877562 |
| 16:30394628-30395027 | 16:30895774-30896508 | 16:30896294-30896859 |
| 16:31129304-31130684 | 16:31202654-31203436 | 16:31471450-31472167 |
| 16:31477471-31478215 | 16:31488551-31489393 | 16:31509035-31509524 |
| 16:31537154-31538147 | 16:49281240-49281775 | 16:50549172-50549704 |
| 16:51150263-51150662 | 16:51151017-51151417 | 16:51152813-51153214 |
| 16:53071565-53072058 | 16:53090792-53092382 | 16:54291603-54292257 |
| 16:54594684-54595084 | 16:54932220-54935384 | 16:54935593-54935994 |
| 16:54936239-54939104 | 16:55478725-55479126 | 16:55480252-55480748 |
| 16:55815098-55815587 | 16:55868899-55870076 | 16:56638121-56638878 |
| 16:56662942-56663517 | 16:56938942-56942012 | 16:57091449-57092190 |
| 16:57166850-57168172 | 16:57178507-57179201 | 16:57283950-57284482 |
| 16:57536986-57537387 | 16:57627456-57628928 | 16:57840866-57841554 |
| 16:57985363-57986243 | 16:60359182-60359602 | 16:62036031-62036430 |
| 16:66366299-66366700 | 16:66578900-66579581 | 16:66603819-66604220 |
| 16:67149722-67151859 | 16:67162750-67163888 | 16:67169170-67171619 |
| 16:67249099-67249707 | 16:67326204-67327956 | 16:67538557-67539214 |
| 16:67969045-67969936 | 16:68084267-68085069 | 16:69089192-69089743 |
| 16:69107196-69107638 | 16:69107507-69108216 | 16:70680310-70681564 |
| 16:70686370-70686907 | 16:70695520-70696116 | 16:71524823-71526598 |
| 16:73066376-73067515 | 16:74965568-74966081 | 16:75000072-75000975 |
| 16:75110574-75111032 | 16:79589550-79589951 | 16:81446775-81447175 |
| 16:81526915-81527495 | 16:84885016-84885416 | 16:85371270-85373056 |
| 16:85395162-85395676 | 16:85637434-85638001 | 16:85637877-85638385 |
| 16:85749780-85750606 | 16:86218847-86219214 | 16:86337491-86338377 |
| 16:86751702-86752234 | 16:86951751-86952739 | 16:87384035-87384641 |
| 16:87464812-87465725 | 16:87490370-87492010 | 16:87607434-87608176 |
| 16:87806057-87806900 | 16:88077543-88077988 | 16:88650575-88651592 |
| 16:89016052-89017071 | 16:89430297-89430907 | 16:89668291-89668962 |
| 17:304468-305404 | 17:335790-336486 | 17:864022-865159 |
| 17:887727-888127 | 17:950531-952223 | 17:1039439-1040058 |
| 17:1228335-1228734 | 17:1298559-1298959 | 17:1574910-1576205 |
| 17:1577159-1577691 | 17:1752504-1753131 | 17:1761777-1764610 |
| 17:1783120-1784417 | 17:1977496-1978189 | 17:2036394-2036900 |
| 17:2711986-2712935 | 17:2723883-2724517 | 17:2815611-2816280 |
| 17:3963569-3964083 | 17:4641479-4642012 | 17:4711197-4711848 |
| 17:4738556-4738956 | 17:4745134-4745931 | 17:4988096-4988630 |
| 17:5097426-5097902 | 17:5115864-5116616 | 17:5499407-5499807 |
| 17:5916690-5917124 | 17:6654594-6656091 | 17:6656148-6656549 |
| 17:6775878-6776461 | 17:6995268-6996589 | 17:7036440-7037063 |
| 17:7080342-7081096 | 17:7392974-7393705 | 17:7438146-7438767 |
| 17:7444802-7445202 | 17:7475885-7478586 | 17:7588804-7589321 |
| 17:7651829-7652243 | 17:7922871-7923271 | 17:8068741-8070714 |
| 17:8153449-8153849 | 17:8310011-8310646 | 17:8326661-8327315 |
| 17:9003606-9004046 | 17:9240278-9240679 | 17:12973759-12974605 |
| 17:15261113-15261513 | 17:15782829-15783309 | 17:15917145-15918144 |
| 17:16666964-16667364 | 17:16837081-16838728 | 17:17723045-17725699 |
| 17:17780547-17781978 | 17:18153598-18153997 | 17:18377304-18377962 |
| 17:19387039-19387527 | 17:19387291-19387957 | 17:19519829-19520666 |
| 17:21377700-21378345 | 17:21463720-21464430 | 17:27457298-27458192 |
| 17:28372299-28372698 | 17:28675756-28676335 | 17:28717000-28717761 |
| 17:28717874-28718681 | 17:29006229-29006629 | 17:29590710-29591470 |
| 17:29613235-29613641 | 17:30234973-30235606 | 17:30922316-30923205 |
| 17:31392072-31392876 | 17:34156811-34157212 | 17:34285505-34286429 |
| 17:34886740-34887256 | 17:35373563-35373962 | 17:35374117-35374518 |
| 17:35752751-35753439 | 17:36464147-36464548 | 17:36657830-36658395 |
| 17:36728516-36728976 | 17:36884386-36884787 | 17:36933676-36934136 |
| 17:36934449-36935437 | 17:36936984-36937384 | 17:36940701-36941102 |
| 17:36941377-36943496 | 17:37491140-37491627 | 17:37824695-37825270 |
| 17:38428843-38429507 | 17:38449400-38449834 | 17:38558416-38558988 |
| 17:38559307-38561009 | 17:38572058-38572596 | 17:38578473-38578873 |
| 17:39201006-39202565 | 17:40069381-40070008 | 17:40177757-40178742 |
| 17:40308816-40310686 | 17:40321328-40322859 | 17:40442639-40443406 |
| 17:40444990-40445714 | 17:40537035-40538484 | 17:40547169-40547929 |
| 17:40664752-40665385 | 17:41441577-41442328 | 17:41521006-41522899 |
| 17:41792807-41793714 | 17:42312149-42312757 | 17:42421408-42422164 |
| 17:42673629-42674344 | 17:43025209-43026100 | 17:43211332-43212485 |
| 17:43530327-43530728 | 17:44308671-44309201 | 17:44325353-44325753 |
| 17:44655995-44656395 | 17:44959966-44960591 | 17:44970214-44970638 |
| 17:45143605-45144359 | 17:45305968-45308048 | 17:45371538-45372069 |
| 17:45785389-45785788 | 17:45896827-45898053 | 17:46266163-46266563 |
| 17:46818563-46819532 | 17:47695209-47696278 | 17:47778222-47778663 |
| 17:47872067-47872662 | 17:48002496-48003359 | 17:48037078-48037478 |
| 17:48526803-48527443 | 17:48544196-48544825 | 17:48578012-48578412 |
| 17:48578265-48578664 | 17:48615010-48615410 | 17:48752612-48753048 |
| 17:48997370-48998069 | 17:48998305-48998705 | 17:49469786-49470643 |
| 17:49523440-49523956 | 17:49994132-49994531 | 17:50168247-50168878 |
| 17:50425455-50425986 | 17:50426462-50427177 | 17:50468438-50471062 |
| 17:50635158-50635713 | 17:50660888-50662452 | 17:52157768-52158183 |
| 17:54899701-54900234 | 17:55265228-55266617 | 17:55722713-55723343 |
| 17:56440368-56441611 | 17:57810940-57811416 | 17:57874579-57875353 |
| 17:58279265-58279802 | 17:58328606-58329084 | 17:58384434-58384889 |
| 17:60138682-60139327 | 17:61252107-61252693 | 17:61402887-61405003 |
| 17:61451974-61452894 | 17:61457335-61458042 | 17:62807979-62808645 |
| 17:63476301-63476895 | 17:63537991-63538702 | 17:63869553-63870245 |
| 17:64777719-64781680 | 17:64781416-64781817 | 17:65228751-65229424 |
| 17:65628480-65628888 | 17:68258973-68259453 | 17:72117084-72118328 |
| 17:72219443-72220341 | 17:72591342-72591712 | 17:73291329-73291977 |
| 17:73308623-73309934 | 17:74213872-74214512 | 17:74234914-74235689 |
| 17:74325809-74326209 | 17:74446581-74447237 | 17:74623539-74624172 |
| 17:75032179-75032897 | 17:75087366-75088927 | 17:75130951-75131352 |
| 17:75587713-75588686 | 17:75645283-75646475 | 17:75688577-75689883 |
| 17:75693902-75696411 | 17:76271919-76272320 | 17:76302585-76303848 |
| 17:76382528-76382991 | 17:76515140-76515741 | 17:76524384-76524828 |
| 17:76586637-76587216 | 17:76710276-76710677 | 17:76840150-76840751 |
| 17:76872330-76872794 | 17:77318848-77319735 | 17:77372517-77372970 |
| 17:77372822-77373292 | 17:77373251-77374679 | 17:77388853-77389500 |
| 17:77391835-77392380 | 17:77417512-77417917 | 17:77420931-77422354 |
| 17:78106162-78106563 | 17:78231764-78232302 | 17:78359406-78359806 |
| 17:78383696-78384500 | 17:78774667-78775458 | 17:78925554-78925955 |
| 17:78993392-78994090 | 17:78994969-78995506 | 17:78997039-78997687 |
| 17:79001022-79001584 | 17:79747059-79747543 | 17:79789349-79790038 |
| 17:79791042-79793755 | 17:79812350-79812749 | 17:79841756-79842757 |
| 17:79978471-79979632 | 17:80067264-80068048 | 17:80832542-80833608 |
| 17:81048802-81049305 | 17:81084108-81085833 | 17:81119921-81120772 |
| 17:81166483-81166883 | 17:81419512-81420068 | 17:81423183-81423928 |
| 17:81536642-81537041 | 17:81647628-81648710 | 17:81811424-81812972 |
| 17:81832029-81832466 | 17:81961551-81962001 | 17:82029702-82030403 |
| 17:82035780-82036259 | 17:82062911-82063379 | 17:82066116-82066601 |
| 17:82090504-82091679 | 17:82092433-82094175 | 17:82094880-82096639 |
| 17:82244303-82245260 | 17:82320797-82321193 | 17:82331371-82332106 |
| 17:82333795-82335139 | 17:82371877-82372278 | 17:82591487-82591935 |
| 17:82848051-82849810 | 17:82968027-82968507 | 17:82986035-82986611 |
| 18:712934-713350 | 18:3498944-3499405 | 18:5543398-5544182 |
| 18:5628093-5631103 | 18:10032781-10033472 | 18:10476445-10477022 |
| 18:10950699-10951509 | 18:11033712-11035231 | 18:11148723-11149123 |
| 18:11149285-11149992 | 18:11663796-11664334 | 18:11751342-11751741 |
| 18:11947215-11948305 | 18:12038228-12038662 | 18:12092433-12093176 |
| 18:12254319-12255127 | 18:12287235-12287712 | 18:12910877-12912280 |
| 18:13611220-13611975 | 18:13641325-13642398 | 18:13826106-13826780 |
| 18:14748100-14748590 | 18:21682464-21683008 | 18:23619400-23619983 |
| 18:23689678-23690534 | 18:23838713-23839314 | 18:23900986-23901505 |
| 18:25921475-25922005 | 18:26546668-26547782 | 18:26550676-26551790 |
| 18:31041373-31042169 | 18:33440436-33441252 | 18:34159087-34159707 |
| 18:37452952-37454413 | 18:45827563-45828939 | 18:46335475-46336088 |
| 18:46757796-46758286 | 18:47248313-47248713 | 18:47251214-47251800 |
| 18:47260967-47261367 | 18:47772968-47773474 | 18:48781038-48781729 |
| 18:48922746-48925291 | 18:48938944-48939517 | 18:49562215-49562740 |
| 18:50282259-50283190 | 18:55585681-55587350 | 18:55954921-55955628 |
| 18:57121582-57121982 | 18:57352259-57352658 | 18:57427794-57428804 |
| 18:57436327-57436727 | 18:57438878-57439279 | 18:57439714-57440154 |
| 18:57440372-57442037 | 18:57728502-57729393 | 18:58195220-58195620 |
| 18:59273863-59274391 | 18:59918379-59918961 | 18:61333327-61334486 |
| 18:61892593-61893524 | 18:62437541-62438111 | 18:62596212-62596613 |
| 18:63317885-63318649 | 18:63949102-63949502 | 18:70288149-70288708 |
| 18:72869075-72869722 | 18:75321316-75321892 | 18:75455316-75455866 |
| 18:75915901-75916965 | 18:76405952-76406498 | 18:77250262-77250866 |
| 18:78879093-78880016 | 18:79506812-79507258 | 18:79575263-79577435 |
| 18:80245911-80246504 | 19:342649-343494 | 19:406018-406761 |
| 19:408460-408861 | 19:709648-710049 | 19:709789-711152 |
| 19:1082847-1083718 | 19:1101475-1102253 | 19:1439719-1440593 |
| 19:1465124-1467459 | 19:1775222-1775623 | 19:1857238-1857638 |
| 19:1992716-1993251 | 19:2252282-2254174 | 19:2282289-2282719 |
| 19:2588080-2588926 | 19:2627289-2628121 | 19:2719742-2720795 |
| 19:3357926-3358420 | 19:3381285-3382073 | 19:3397571-3399284 |
| 19:3687834-3688735 | 19:3977568-3978412 | 19:3979687-3981485 |
| 19:3982456-3983423 | 19:4379879-4380577 | 19:4715398-4715917 |
| 19:5338435-5339343 | 19:5823546-5823976 | 19:6057103-6057820 |
| 19:6674795-6675276 | 19:6767804-6768205 | 19:7617645-7618307 |
| 19:7620118-7620744 | 19:7861746-7863238 | 19:7928317-7929639 |
| 19:9362767-9363166 | 19:9498520-9498919 | 19:9945549-9945998 |
| 19:10086869-10087481 | 19:10286751-10287150 | 19:11142602-11143753 |
| 19:11420435-11420835 | 19:11578522-11579050 | 19:11674078-11674478 |
| 19:12752387-12753623 | 19:12867290-12868042 | 19:12880700-12881406 |
| 19:12933958-12934600 | 19:13163828-13165681 | 19:13978215-13979739 |
| 19:14003036-14003653 | 19:14204719-14205216 | 19:14799867-14801180 |
| 19:15123919-15125182 | 19:15231787-15232321 | 19:15232212-15232734 |
| 19:15232824-15233385 | 19:15233277-15233703 | 19:15469306-15470060 |
| 19:15911511-15912274 | 19:16075801-16078469 | 19:16325512-16326702 |
| 19:16570235-16571654 | 19:16888584-16888983 | 19:16888772-16889173 |
| 19:17027046-17027447 | 19:17135342-17136125 | 19:17328772-17329589 |
| 19:17847673-17848166 | 19:17861146-17862292 | 19:18023770-18024436 |
| 19:18118988-18119537 | 19:18304552-18305342 | 19:18428795-18429670 |
| 19:18434102-18434909 | 19:18436203-18437397 | 19:18479666-18480405 |
| 19:18790883-18791458 | 19:18918127-18918658 | 19:18985464-18985996 |
| 19:19110344-19110745 | 19:19262561-19262961 | 19:19272744-19273317 |
| 19:19860619-19861267 | 19:21483785-21485055 | 19:22622848-22623532 |
| 19:22807113-22807726 | 19:23002549-23003605 | 19:29411673-29414227 |
| 19:29645212-29645775 | 19:31339882-31340442 | 19:32104826-32105225 |
| 19:32824210-32824857 | 19:33820271-33821105 | 19:33906383-33906927 |
| 19:34577288-34578190 | 19:34904745-34905721 | 19:35533632-35534050 |
| 19:35544750-35545151 | 19:36032892-36033306 | 19:36245218-36245725 |
| 19:38209543-38209997 | 19:38209741-38210202 | 19:38211153-38211554 |
| 19:38256347-38256747 | 19:38256526-38256926 | 19:38394373-38396300 |
| 19:38418217-38418874 | 19:38770113-38770514 | 19:38869522-38869975 |
| 19:38975177-38975692 | 19:38975522-38976267 | 19:39203826-39204341 |
| 19:40365499-40367038 | 19:40487655-40488234 | 19:40613522-40614722 |
| 19:40810755-40812481 | 19:40848497-40848927 | 19:41027048-41029566 |
| 19:41089915-41090315 | 19:41376586-41376986 | 19:41428114-41428558 |
| 19:42199304-42199974 | 19:42281090-42281560 | 19:42422771-42423392 |
| 19:42738663-42742095 | 19:42754786-42756103 | 19:42926160-42926674 |
| 19:43152293-43153775 | 19:43634470-43635723 | 19:43699446-43699911 |
| 19:45385755-45386356 | 19:45402130-45403049 | 19:45472442-45473154 |
| 19:45876354-45877026 | 19:45883881-45885031 | 19:46296616-46297604 |
| 19:46298149-46298565 | 19:46411871-46414104 | 19:46648363-46650142 |
| 19:47004003-47004404 | 19:47232476-47233127 | 19:47239207-47239854 |
| 19:47573369-47573821 | 19:48061590-48062217 | 19:48480051-48481211 |
| 19:48612804-48613718 | 19:48615742-48618249 | 19:48696557-48697073 |
| 19:49058099-49059629 | 19:49200474-49200875 | 19:49207586-49208420 |
| 19:49432401-49432800 | 19:49489718-49491072 | 19:49512115-49512868 |
| 19:50202878-50203296 | 19:50728327-50728733 | 19:50800709-50801109 |
| 19:50856467-50858276 | 19:50872486-50874876 | 19:50912603-50913411 |
| 19:51019047-51020146 | 19:51064564-51065154 | 19:51064928-51065999 |
| 19:51326856-51327257 | 19:51340196-51340751 | 19:51688014-51688415 |
| 19:51719294-51719996 | 19:51887305-51888551 | 19:51900935-51902306 |
| 19:51975668-51976324 | 19:51977213-51977666 | 19:52492472-52493642 |
| 19:52535304-52536810 | 19:53785216-53785686 | 19:53864259-53864948 |
| 19:53865987-53866387 | 19:53941972-53942784 | 19:54019964-54020652 |
| 19:54465119-54465519 | 19:54472974-54473711 | 19:54573164-54575127 |
| 19:55080823-55081712 | 19:55087259-55087660 | 19:55452344-55453365 |
| 19:56253642-56254160 | 19:56642783-56644076 | 19:56671447-56671848 |
| 19:57708451-57709795 | 19:57830115-57830981 | 19:58538325-58539063 |
| 2:45250-45968 | 2:287054-287933 | 2:951272-952142 |
| 2:1687139-1687562 | 2:1728249-1728725 | 2:2698571-2699158 |
| 2:3702800-3703812 | 2:3786091-3786914 | 2:5673367-5673884 |
| 2:5690864-5691412 | 2:5693491-5694080 | 2:5696430-5697447 |
| 2:5981632-5982125 | 2:8457058-8457459 | 2:8534793-8535466 |
| 2:8583705-8584105 | 2:9095606-9096005 | 2:9153222-9153688 |
| 2:9753147-9754535 | 2:9757876-9758413 | 2:10091208-10091869 |
| 2:10228402-10228914 | 2:10365772-10366867 | 2:10912340-10912982 |
| 2:11677345-11677842 | 2:12456232-12456917 | 2:12718206-12719244 |
| 2:17517825-17518571 | 2:17518292-17518692 | 2:17537867-17538418 |
| 2:17538989-17539641 | 2:17877649-17878049 | 2:19361412-19362197 |
| 2:19868472-19869295 | 2:20008742-20009166 | 2:20665740-20666497 |
| 2:20822520-20823267 | 2:23649724-23650233 | 2:24010867-24011754 |
| 2:24926602-24927429 | 2:25168732-25169132 | 2:25216090-25216656 |
| 2:25279064-25279698 | 2:25339937-25340422 | 2:25981308-25982115 |
| 2:26172339-26172837 | 2:26173767-26174529 | 2:26298353-26299392 |
| 2:26299935-26301519 | 2:26401648-26402329 | 2:27306078-27308818 |
| 2:27735124-27735894 | 2:27735650-27736049 | 2:28810763-28811336 |
| 2:29114929-29115542 | 2:29213796-29214357 | 2:31067732-31069149 |
| 2:33133980-33134440 | 2:37586209-37586610 | 2:39620072-39620715 |
| 2:39665159-39665622 | 2:42047274-42047674 | 2:42102150-42102550 |
| 2:42792193-42794092 | 2:42933810-42934363 | 2:43005876-43006464 |
| 2:43223675-43224879 | 2:43227756-43228156 | 2:43637623-43638066 |
| 2:44286325-44287001 | 2:44801714-44802296 | 2:44802618-44803083 |
| 2:44929647-44930307 | 2:44932803-44933264 | 2:44933128-44933528 |
| 2:44942258-44942692 | 2:44944331-44945180 | 2:44949573-44950136 |
| 2:45003898-45005327 | 2:45010349-45010750 | 2:45013273-45014233 |
| 2:45652275-45652901 | 2:46299276-46300109 | 2:46814015-46814518 |
| 2:48529246-48529728 | 2:50347745-50348247 | 2:54673489-54674237 |
| 2:54709428-54709989 | 2:55922959-55923493 | 2:60530594-60531099 |
| 2:60717746-60719842 | 2:62475080-62475649 | 2:62570579-62571463 |
| 2:63053769-63054168 | 2:63056681-63057783 | 2:63057811-63060097 |
| 2:64946058-64946600 | 2:65640212-65640717 | 2:66435455-66435854 |
| 2:66436532-66438446 | 2:66439009-66440077 | 2:66507922-66509218 |
| 2:68318667-68319066 | 2:69798456-69799068 | 2:70085880-70086435 |
| 2:70862213-70862652 | 2:70888089-70888700 | 2:70888906-70889431 |
| 2:70907243-70907956 | 2:70978283-70979311 | 2:71070216-71071973 |
| 2:72924177-72924616 | 2:73071819-73072218 | 2:74148161-74148560 |
| 2:74414809-74415494 | 2:74497666-74500107 | 2:74514981-74515382 |
| 2:74515508-74516032 | 2:74516375-74517010 | 2:74647917-74648318 |
| 2:74648025-74648424 | 2:74654673-74655156 | 2:80301921-80303552 |
| 2:84879679-84880347 | 2:84880143-84880543 | 2:85413488-85413970 |
| 2:85413802-85414281 | 2:85765303-85765855 | 2:85886498-85887704 |
| 2:86788639-86789885 | 2:88128812-88129213 | 2:88350580-88351169 |
| 2:88860418-88862171 | 2:95026619-95027136 | 2:96146573-96147356 |
| 2:96324827-96326349 | 2:96485686-96486535 | 2:96527120-96528052 |
| 2:96532179-96532579 | 2:96762703-96764094 | 2:96839387-96839877 |
| 2:98347572-98348123 | 2:98822918-98823570 | 2:98936792-98937329 |
| 2:99179211-99179799 | 2:101004004-101004857 | 2:101135387-101136247 |
| 2:101474542-101475010 | 2:102186679-102187477 | 2:102418618-102420037 |
| 2:104842490-104843467 | 2:104853949-104854486 | 2:104857770-104858170 |
| 2:105398292-105398693 | 2:106587147-106587872 | 2:107986336-107986735 |
| 2:108301529-108301971 | 2:108377511-108378426 | 2:108606932-108609282 |
| 2:108957111-108957624 | 2:109615043-109616460 | 2:110120644-110121055 |
| 2:111494357-111494913 | 2:112085176-112086177 | 2:112181733-112182269 |
| 2:112588753-112589269 | 2:112690591-112691627 | 2:112836615-112837184 |
| 2:113503641-113504552 | 2:115161017-115161582 | 2:115162029-115162430 |
| 2:117836125-117836902 | 2:118848969-118849370 | 2:119157128-119159498 |
| 2:119431134-119431627 | 2:120176762-120177350 | 2:120587002-120587723 |
| 2:120654278-120654713 | 2:120669604-120670003 | 2:120867296-120868209 |
| 2:121225704-121226341 | 2:121283923-121284919 | 2:122660983-122661667 |
| 2:124024481-124024880 | 2:126888677-126889166 | 2:127025391-127025792 |
| 2:127218600-127218999 | 2:128318619-128319018 | 2:128490017-128490399 |
| 2:128584637-128585043 | 2:129992864-129993944 | 2:130005703-130006341 |
| 2:130213439-130213920 | 2:130372694-130373095 | 2:130427511-130429192 |
| 2:130691952-130693980 | 2:130755044-130755548 | 2:130837383-130838067 |
| 2:130915927-130916727 | 2:131300158-131300716 | 2:131331077-131331489 |
| 2:131394572-131395006 | 2:131528248-131529007 | 2:132256179-132257169 |
| 2:132670928-132671877 | 2:134027121-134028424 | 2:136989900-136991413 |
| 2:138779394-138779793 | 2:143035981-143036435 | 2:147843459-147843980 |
| 2:148887652-148888379 | 2:149160240-149161222 | 2:149588083-149588484 |
| 2:151947351-151947822 | 2:152421496-152422660 | 2:153477785-153478287 |
| 2:154698753-154700489 | 2:156320409-156320983 | 2:156321273-156322527 |
| 2:157327183-157327952 | 2:157453173-157453988 | 2:159904423-159904901 |
| 2:160491801-160492421 | 2:161136146-161137004 | 2:161237406-161237807 |
| 2:161416664-161417163 | 2:161416986-161418063 | 2:161422616-161424536 |
| 2:161426527-161427073 | 2:161427043-161427495 | 2:161427332-161427774 |
| 2:161427930-161428329 | 2:161625373-161625928 | 2:164993297-164994150 |
| 2:170712130-170712772 | 2:170716053-170717059 | 2:171521915-171522365 |
| 2:172092704-172093759 | 2:172096401-172096926 | 2:172100167-172100702 |
| 2:172985290-172985838 | 2:174325990-174327238 | 2:174327936-174328799 |
| 2:174333013-174333702 | 2:174334741-174335142 | 2:174343709-174344183 |
| 2:174683141-174683947 | 2:174696247-174696735 | 2:176075348-176075894 |
| 2:176081112-176085695 | 2:176103705-176105769 | 2:176156823-176157248 |
| 2:176158083-176159182 | 2:176159293-176159716 | 2:176160068-176161397 |
| 2:176162130-176162531 | 2:176162561-176163043 | 2:176162831-176163353 |
| 2:176164194-176165235 | 2:176164971-176165650 | 2:176174547-176174990 |
| 2:176189427-176189995 | 2:179860059-179860840 | 2:179861769-179862170 |
| 2:181456611-181457278 | 2:181678305-181678706 | 2:187554095-187554774 |
| 2:189178115-189180266 | 2:190180103-190180854 | 2:190180755-190181309 |
| 2:190343239-190343656 | 2:191262587-191263307 | 2:191845108-191845960 |
| 2:191846932-191847582 | 2:200585449-200586389 | 2:201643820-201644459 |
| 2:202032023-202032433 | 2:202035139-202035540 | 2:203951410-203952041 |
| 2:205609143-205609544 | 2:205686337-205686892 | 2:206444198-206444651 |
| 2:207627064-207627483 | 2:207681162-207681561 | 2:208124372-208124851 |
| 2:212536152-212537056 | 2:214810230-214810944 | 2:216005067-216006835 |
| 2:216535255-216535802 | 2:217247378-217248984 | 2:217850428-217851913 |
| 2:217905336-217905797 | 2:217943665-217944563 | 2:218033508-218034222 |
| 2:218164539-218165074 | 2:218781607-218782787 | 2:218806282-218806683 |
| 2:218858747-218859239 | 2:218981298-218983753 | 2:218983500-218983956 |
| 2:219057439-219057840 | 2:219252726-219253373 | 2:219309319-219309719 |
| 2:219418263-219418663 | 2:219569956-219570533 | 2:221104331-221104727 |
| 2:221570343-221571139 | 2:222289220-222289621 | 2:222291055-222291567 |
| 2:222298005-222298757 | 2:222298877-222299277 | 2:222299931-222300330 |
| 2:222311725-222312125 | 2:224039817-224040314 | 2:224401944-224402344 |
| 2:226423460-226423952 | 2:226797218-226798523 | 2:227717619-227718122 |
| 2:227871278-227871883 | 2:228180065-228180455 | 2:230814103-230814635 |
| 2:231396095-231396496 | 2:231614813-231615276 | 2:232503402-232503983 |
| 2:232525866-232526265 | 2:232633764-232634723 | 2:232876778-232877492 |
| 2:233413410-233413864 | 2:233475611-233476912 | 2:234446582-234447088 |
| 2:234497439-234497839 | 2:235106861-235107266 | 2:236169372-236169869 |
| 2:236169762-236170240 | 2:236171460-236171860 | 2:236172856-236173580 |
| 2:236507510-236507911 | 2:237591287-237592062 | 2:237626669-237627224 |
| 2:237702384-237703039 | 2:237707401-237707931 | 2:238170350-238170750 |
| 2:238231390-238231827 | 2:238289504-238290354 | 2:239052156-239053108 |
| 2:239109607-239110089 | 2:239221838-239223133 | 2:239358130-239358712 |
| 2:240557262-240557793 | 2:240818576-240819538 | 2:240916432-240916876 |
| 2:241036188-241036813 | 2:241540606-241541239 | 2:241546797-241547198 |
| 2:241609865-241610678 | 2:241678369-241679152 | 2:241962402-241962801 |
| 20:309935-310589 | 20:610272-610673 | 20:642392-642927 |
| 20:661641-662400 | 20:710120-710604 | 20:1895391-1896969 |
| 20:2654079-2655564 | 20:2691678-2692473 | 20:2799491-2801252 |
| 20:3071686-3072541 | 20:3237598-3238448 | 20:3239869-3240594 |
| 20:3777351-3777835 | 20:4083135-4083639 | 20:4822503-4822994 |
| 20:5292158-5292841 | 20:5301935-5302457 | 20:5445732-5446626 |
| 20:6051985-6052709 | 20:6214230-6215405 | 20:6769819-6770642 |
| 20:8789956-8790418 | 20:9515708-9516109 | 20:9516094-9516833 |
| 20:10295870-10296453 | 20:10511457-10511919 | 20:18508629-18509166 |
| 20:20365321-20365722 | 20:20366748-20367149 | 20:21100959-21101410 |
| 20:21103578-21103979 | 20:21106233-21106634 | 20:21512030-21512430 |
| 20:21516391-21517806 | 20:23035084-23035485 | 20:24577880-24578391 |
| 20:24800834-24802143 | 20:24814562-24816945 | 20:25036060-25037451 |
| 20:25148508-25149076 | 20:25469970-25470475 | 20:25584567-25585067 |
| 20:25585595-25586077 | 20:26207851-26208422 | 20:26209505-26209904 |
| 20:31588076-31588477 | 20:31607964-31608442 | 20:31870680-31871263 |
| 20:32040160-32040942 | 20:32108873-32109364 | 20:32778455-32778855 |
| 20:34269115-34269571 | 20:35600706-35601264 | 20:36091594-36092233 |
| 20:37295594-37297339 | 20:37357856-37358366 | 20:37384342-37384743 |
| 20:37567197-37567713 | 20:37598217-37598618 | 20:38152459-38153107 |
| 20:38298710-38299165 | 20:38601182-38602120 | 20:38674557-38674991 |
| 20:38806683-38807223 | 20:41366811-41367211 | 20:43508218-43508617 |
| 20:43915295-43916176 | 20:43915921-43916320 | 20:44187340-44187740 |
| 20:44246974-44247443 | 20:44326681-44327168 | 20:45097752-45098274 |
| 20:45100735-45101370 | 20:45114944-45115381 | 20:45406886-45408100 |
| 20:45469432-45470929 | 20:45890006-45890604 | 20:45912173-45912574 |
| 20:46029923-46030754 | 20:46057198-46057599 | 20:46099372-46100406 |
| 20:46117985-46118384 | 20:46458975-46459501 | 20:46550367-46550968 |
| 20:46650994-46651870 | 20:46810321-46812040 | 20:47872457-47873396 |
| 20:47894614-47896185 | 20:48563450-48563938 | 20:48722800-48723305 |
| 20:49567462-49568011 | 20:50308245-50308943 | 20:51492109-51493139 |
| 20:51631136-51632011 | 20:52104041-52106011 | 20:52973040-52973596 |
| 20:53581402-53582201 | 20:53608650-53609978 | 20:53660257-53660661 |
| 20:53897976-53899875 | 20:54222535-54223023 | 20:56004203-56005665 |
| 20:56631087-56632206 | 20:56721711-56722280 | 20:56821277-56821852 |
| 20:57040763-57041287 | 20:57266681-57267166 | 20:57322969-57323526 |
| 20:57398249-57398854 | 20:57664431-57664831 | 20:57675870-57676805 |
| 20:57679317-57681685 | 20:57682561-57683352 | 20:57690430-57692206 |
| 20:57711236-57712497 | 20:58135519-58136129 | 20:58228151-58228872 |
| 20:58514201-58515959 | 20:59006194-59006625 | 20:61043138-61044110 |
| 20:61529537-61530079 | 20:61544273-61544673 | 20:61870693-61871170 |
| 20:61951742-61952349 | 20:61965181-61965644 | 20:62603664-62604109 |
| 20:62686691-62687284 | 20:62952407-62952906 | 20:62954371-62954772 |
| 20:63086352-63086926 | 20:63487938-63488339 | 20:63488479-63489352 |
| 20:63499542-63500081 | 20:63737452-63738301 | 20:63741921-63742964 |
| 20:63829655-63830367 | 20:63919099-63920103 | 20:63930979-63931764 |
| 20:64062425-64062824 | 20:64163837-64164485 | 21:16194248-16194764 |
| 21:17689372-17689897 | 21:20997333-20998069 | 21:25562113-25562723 |
| 21:31559715-31560364 | 21:32413189-32413961 | 21:33023908-33027361 |
| 21:33071647-33072089 | 21:33078974-33079509 | 21:33383035-33383861 |
| 21:33402425-33402889 | 21:34669720-34670195 | 21:37005607-37006484 |
| 21:37208328-37208867 | 21:37547171-37547580 | 21:38925903-38926389 |
| 21:38984674-38986362 | 21:39105418-39106291 | 21:39345495-39346999 |
| 21:41113203-41113783 | 21:41426516-41426970 | 21:41481176-41481726 |
| 21:41774762-41775195 | 21:41885044-41885709 | 21:41921123-41922160 |
| 21:42616277-42616733 | 21:43333742-43334209 | 21:43361655-43362767 |
| 21:43728300-43729411 | 21:43903405-43904206 | 21:44479419-44480087 |
| 21:45403734-45404728 | 21:45477779-45479161 | 21:45662383-45663160 |
| 21:46224749-46225577 | 21:46297341-46298505 | 22:16601540-16602032 |
| 22:16615213-16616201 | 22:17121520-17121941 | 22:17366273-17368019 |
| 22:17785007-17786015 | 22:19148538-19149303 | 22:19524033-19524947 |
| 22:19766451-19767075 | 22:19879029-19879553 | 22:19935253-19937426 |
| 22:20013393-20013787 | 22:20509295-20509775 | 22:20957899-20958300 |
| 22:21013851-21014280 | 22:21651179-21651580 | 22:21652470-21653390 |
| 22:23282140-23283212 | 22:24494979-24495379 | 22:24806481-24806860 |
| 22:24837492-24838188 | 22:24901671-24902180 | 22:25112618-25113862 |
| 22:26567943-26568457 | 22:27802079-27802753 | 22:28441786-28443829 |
| 22:29208913-29209507 | 22:29308194-29308921 | 22:29581520-29581920 |
| 22:30252963-30253750 | 22:31662530-31662987 | 22:35328460-35329018 |
| 22:35838465-35839398 | 22:36978449-36979053 | 22:37324455-37325414 |
| 22:37675363-37677667 | 22:37818390-37819122 | 22:37943795-37944218 |
| 22:38034053-38034604 | 22:38569589-38570079 | 22:38701526-38701927 |
| 22:39014039-39014657 | 22:39040002-39040998 | 22:39076948-39077541 |
| 22:39235112-39235796 | 22:39314728-39315803 | 22:39316750-39317433 |
| 22:39349345-39349688 | 22:39363641-39364475 | 22:39567319-39567801 |
| 22:40323872-40325000 | 22:41238304-41239649 | 22:41910082-41911097 |
| 22:41910798-41912177 | 22:41920090-41920628 | 22:42299872-42300308 |
| 22:42431968-42432555 | 22:42551819-42552665 | 22:42644485-42645290 |
| 22:43109616-43110221 | 22:43263610-43264639 | 22:44676506-44677378 |
| 22:45226677-45227250 | 22:45239533-45240015 | 22:45271691-45272180 |
| 22:45503155-45504982 | 22:45531748-45534009 | 22:45865924-45866790 |
| 22:45971172-45971887 | 22:46374134-46374789 | 22:46538358-46539778 |
| 22:47631741-47632165 | 22:49302344-49302957 | 22:49501548-49502774 |
| 22:49849680-49850542 | 22:50105741-50106331 | 22:50115731-50116208 |
| 22:50170979-50171474 | 22:50185060-50185460 | 22:50299286-50300178 |
| 22:50529652-50530051 | 22:50539510-50540144 | 3:3666147-3666734 |
| 3:4736369-4737007 | 3:4740559-4741588 | 3:4751537-4752606 |
| 3:4950956-4951490 | 3:5002344-5002724 | 3:5095794-5096635 |
| 3:9902298-9903168 | 3:11582177-11582765 | 3:11638821-11639386 |
| 3:12796918-12797528 | 3:14123453-14124045 | 3:14534458-14534902 |
| 3:14555661-14556373 | 3:14811001-14811548 | 3:23750943-23751768 |
| 3:25427483-25429011 | 3:27723703-27724755 | 3:29281400-29281977 |
| 3:32818535-32819125 | 3:33278000-33278551 | 3:33658970-33659917 |
| 3:35664385-35665141 | 3:36764172-36764838 | 3:37452701-37454516 |
| 3:37993821-37994693 | 3:38028702-38030449 | 3:38953480-38954076 |
| 3:40387473-40388310 | 3:42015020-42015472 | 3:42264850-42266457 |
| 3:42905742-42906348 | 3:43995701-43998520 | 3:44184208-44184671 |
| 3:44554717-44555196 | 3:44584780-44585180 | 3:44585054-44585518 |
| 3:45553273-45553890 | 3:45677112-45677513 | 3:46464458-46465314 |
| 3:46665328-46665791 | 3:46947223-46947753 | 3:47009103-47010331 |
| 3:48507655-48508231 | 3:48594187-48595500 | 3:50205658-50206058 |
| 3:50237044-50237532 | 3:50340709-50341126 | 3:50340843-50341246 |
| 3:51956134-51956772 | 3:52246116-52247425 | 3:52299995-52300531 |
| 3:52534228-52535383 | 3:52688937-52689678 | 3:52842994-52843553 |
| 3:53270027-53270756 | 3:53495209-53495778 | 3:54087751-54088155 |
| 3:54120478-54120960 | 3:54966877-54967292 | 3:55489638-55490767 |
| 3:57164044-57164919 | 3:57959735-57960507 | 3:59451199-59451752 |
| 3:61563580-61565053 | 3:62318617-62319017 | 3:62365555-62366126 |
| 3:62367486-62368369 | 3:62369115-62369516 | 3:62369493-62369918 |
| 3:62369699-62370359 | 3:62371136-62371651 | 3:62373947-62374637 |
| 3:62374591-62375368 | 3:62376956-62377551 | 3:62377640-62378552 |
| 3:64214128-64214594 | 3:64267588-64268395 | 3:64684506-64685459 |
| 3:65597099-65598092 | 3:66404106-66404621 | 3:68931018-68932124 |
| 3:69012054-69012763 | 3:69919999-69920395 | 3:71070095-71070601 |
| 3:71152764-71154030 | 3:72177663-72178062 | 3:72288852-72289368 |
| 3:72738542-72739309 | 3:77097807-77098278 | 3:80770287-80771487 |
| 3:84961325-84961726 | 3:87088823-87089826 | 3:98778553-98779323 |
| 3:99875898-99876332 | 3:99876150-99876560 | 3:111610237-111610829 |
| 3:111999247-111999868 | 3:112303998-112304475 | 3:112332804-112333203 |
| 3:112332993-112333866 | 3:112641954-112642537 | 3:112749306-112750725 |
| 3:113276263-113276748 | 3:115658746-115659242 | 3:116445490-116445945 |
| 3:119575820-119576287 | 3:120284294-120285005 | 3:121660795-121661837 |
| 3:122324801-122325346 | 3:122577439-122578169 | 3:123025990-123026863 |
| 3:123883487-123884098 | 3:124780570-124781452 | 3:124860851-124861263 |
| 3:124901584-124901974 | 3:125051964-125052910 | 3:125141621-125142177 |
| 3:125928843-125929802 | 3:126179636-126180251 | 3:126180553-126181552 |
| 3:126287768-126288793 | 3:126752564-126753554 | 3:126911406-126911981 |
| 3:126984088-126984998 | 3:128845590-128846433 | 3:129000380-129001472 |
| 3:129002309-129003642 | 3:129045830-129046247 | 3:129305542-129306146 |
| 3:129627107-129627595 | 3:129974376-129974920 | 3:132033664-132034065 |
| 3:132034250-132035437 | 3:133674458-133674962 | 3:133745720-133746496 |
| 3:134028821-134029292 | 3:134363870-134364729 | 3:134406333-134407307 |
| 3:136818614-136819017 | 3:136819376-136820242 | 3:136820101-136820776 |
| 3:138112328-138114888 | 3:138435240-138436063 | 3:138936746-138940415 |
| 3:138943272-138943673 | 3:138944237-138944708 | 3:138947057-138947456 |
| 3:138960354-138960886 | 3:139538872-139539474 | 3:139539707-139540743 |
| 3:139559658-139560153 | 3:141077016-141077416 | 3:141962462-141963602 |
| 3:142724844-142725510 | 3:142964076-142964842 | 3:147390596-147394590 |
| 3:147407724-147408125 | 3:147409024-147409423 | 3:147409633-147410033 |
| 3:147413344-147413745 | 3:152267601-152268094 | 3:154427778-154430514 |
| 3:157436523-157437135 | 3:157542613-157543149 | 3:158093970-158095850 |
| 3:158101827-158104573 | 3:158732268-158733194 | 3:160038710-160039156 |
| 3:160224243-160225195 | 3:160450040-160450681 | 3:165197207-165197608 |
| 3:169811720-169812120 | 3:170036931-170037398 | 3:170585077-170586407 |
| 3:170907551-170908283 | 3:171028006-171028653 | 3:172129543-172130928 |
| 3:173398107-173398620 | 3:177357364-177358587 | 3:179649378-179649875 |
| 3:180616467-180617016 | 3:181695908-181696383 | 3:181719074-181720624 |
| 3:181726818-181729551 | 3:183161797-183162197 | 3:183264981-183265552 |
| 3:184275772-184276268 | 3:184380974-184381932 | 3:184601478-184601928 |
| 3:185715403-185715943 | 3:186193270-186193809 | 3:186210130-186210691 |
| 3:186242304-186242979 | 3:186360630-186361246 | 3:186772404-186773138 |
| 3:187670060-187670510 | 3:187734926-187738205 | 3:190533440-190534749 |
| 3:192408083-192408526 | 3:193379421-193380240 | 3:194058028-194058691 |
| 3:194089428-194089930 | 3:194206426-194206863 | 3:194285278-194285742 |
| 3:195105531-195106006 | 3:195859827-195860461 | 3:196023702-196024103 |
| 3:196096611-196097038 | 3:196121950-196122461 | 3:196179310-196180422 |
| 3:197028668-197029310 | 3:197712154-197712754 | 4:304338-305673 |
| 4:386406-386807 | 4:389241-390421 | 4:688787-689421 |
| 4:694531-695019 | 4:785539-787528 | 4:1204442-1205554 |
| 4:1207944-1209244 | 4:1249910-1250368 | 4:1348222-1349256 |
| 4:1404660-1405160 | 4:1413755-1414351 | 4:1578235-1578635 |
| 4:1718306-1718862 | 4:1752085-1752664 | 4:2021578-2022177 |
| 4:2058184-2058585 | 4:2430059-2430885 | 4:2798121-2798718 |
| 4:2800264-2801560 | 4:3384839-3386506 | 4:3388755-3389729 |
| 4:3483385-3483816 | 4:4168136-4168931 | 4:4855026-4855505 |
| 4:4858116-4858516 | 4:4860892-4861705 | 4:5051622-5052023 |
| 4:5708437-5708838 | 4:6447016-6447572 | 4:6658892-6659368 |
| 4:6663688-6664432 | 4:7960657-7961224 | 4:7965171-7965793 |
| 4:8228272-8229317 | 4:8269513-8269930 | 4:8691404-8691825 |
| 4:9781263-9782158 | 4:11427047-11428216 | 4:13522266-13522832 |
| 4:13529148-13529721 | 4:13531152-13531671 | 4:13534729-13535517 |
| 4:13537198-13537599 | 4:13538898-13539472 | 4:13543864-13544286 |
| 4:15702776-15703322 | 4:15778225-15779049 | 4:16083278-16084740 |
| 4:16360549-16361015 | 4:17781557-17782030 | 4:20882411-20883004 |
| 4:22997258-22997658 | 4:23945468-23946155 | 4:25655387-25655909 |
| 4:25655641-25656156 | 4:25788005-25788605 | 4:37976891-37977290 |
| 4:39455440-39456782 | 4:40301572-40302053 | 4:41256392-41257177 |
| 4:41744446-41747668 | 4:41751400-41751845 | 4:41864080-41865747 |
| 4:41866918-41867712 | 4:41873025-41874109 | 4:41878072-41881240 |
| 4:46389761-46390386 | 4:46909206-46909683 | 4:46992805-46993970 |
| 4:47837708-47838295 | 4:48127909-48128516 | 4:48489954-48490437 |
| 4:48490629-48491029 | 4:52050956-52051394 | 4:54093101-54093531 |
| 4:54093523-54094215 | 4:54099511-54099982 | 4:54103564-54104485 |
| 4:54109359-54110167 | 4:54226700-54227099 | 4:54227641-54228040 |
| 4:54656997-54657699 | 4:55124576-55124977 | 4:56129414-56129843 |
| 4:56505272-56506581 | 4:56656176-56656865 | 4:56757596-56758023 |
| 4:57110192-57110592 | 4:57110439-57111253 | 4:73704190-73704973 |
| 4:73868846-73869357 | 4:73943847-73944744 | 4:73998297-73999045 |
| 4:74098467-74098978 | 4:76195922-76196374 | 4:76421515-76422069 |
| 4:80185358-80186083 | 4:80263548-80264224 | 4:80268572-80268973 |
| 4:81214302-81214780 | 4:81505316-81506415 | 4:82797598-82797999 |
| 4:84480839-84482029 | 4:84482611-84483096 | 4:84483827-84484248 |
| 4:84492712-84493144 | 4:84493182-84493870 | 4:84495550-84497813 |
| 4:84502984-84503466 | 4:85929921-85930422 | 4:86358866-86360658 |
| 4:89835754-89836785 | 4:93827300-93827700 | 4:93834218-93835030 |
| 4:98659138-98659538 | 4:100190564-100191310 | 4:103719110-103720548 |
| 4:105145438-105146360 | 4:107760405-107760973 | 4:109301666-109302182 |
| 4:112509349-112509983 | 4:113978743-113979426 | 4:121072752-121073152 |
| 4:121380915-121381315 | 4:121711468-121711913 | 4:121765119-121765519 |
| 4:122700496-122701601 | 4:122825993-122826760 | 4:123772763-123773243 |
| 4:125317048-125317447 | 4:127623551-127624379 | 4:127783212-127783646 |
| 4:134621729-134622172 | 4:138851323-138852365 | 4:139279215-139280096 |
| 4:139657487-139658292 | 4:140497863-140498272 | 4:140567659-140568110 |
| 4:146639850-146640251 | 4:147481546-147481947 | 4:152045828-152046413 |
| 4:152680016-152680416 | 4:153222983-153223798 | 4:153759763-153760321 |
| 4:153788501-153788900 | 4:153789010-153789565 | 4:153789459-153789858 |
| 4:153790995-153793413 | 4:155376976-155377376 | 4:157221615-157223180 |
| 4:163932468-163932882 | 4:167905845-167906246 | 4:168634120-168634908 |
| 4:168832949-168833641 | 4:168848279-168849236 | 4:171812911-171814803 |
| 4:173500889-173501441 | 4:173506708-173507109 | 4:173509185-173509613 |
| 4:173529420-173530140 | 4:174211505-174211905 | 4:177908023-177908486 |
| 4:182140490-182143630 | 4:183796742-183797399 | 4:184384809-184386762 |
| 4:184417220-184417727 | 4:185016425-185016859 | 4:185513688-185514558 |
| 4:185820050-185820450 | 4:186538108-186538508 | 4:187995275-187995722 |
| 4:187995550-187995949 | 4:188282680-188283470 | 4:188939523-188939923 |
| 4:189472850-189475176 | 5:272791-274675 | 5:399650-400772 |
| 5:528314-528997 | 5:600511-600912 | 5:663937-664595 |
| 5:690895-691615 | 5:782938-783339 | 5:1021478-1022000 |
| 5:1093316-1093838 | 5:1445897-1446762 | 5:1593861-1594330 |
| 5:1851298-1851864 | 5:1874748-1876433 | 5:1930486-1930887 |
| 5:1931355-1932134 | 5:2755555-2756149 | 5:3595705-3596740 |
| 5:5033729-5034340 | 5:5047003-5048785 | 5:6583116-6583638 |
| 5:7849502-7850475 | 5:9546412-9546813 | 5:9547205-9548136 |
| 5:10467658-10468189 | 5:10563095-10563539 | 5:10741554-10742131 |
| 5:10761360-10761761 | 5:11588698-11589878 | 5:11902947-11903383 |
| 5:14872674-14873080 | 5:32027794-32028527 | 5:32712366-32714643 |
| 5:33891530-33892750 | 5:35990744-35991672 | 5:36066530-36067139 |
| 5:36657430-36658042 | 5:38257263-38258076 | 5:38846445-38847147 |
| 5:40680417-40682066 | 5:41509793-41510194 | 5:42812083-42813690 |
| 5:42943948-42944559 | 5:42951814-42954954 | 5:43191642-43192084 |
| 5:43193331-43193905 | 5:43603390-43604197 | 5:50966688-50967112 |
| 5:50967515-50967998 | 5:50969458-50970415 | 5:51382416-51382817 |
| 5:53482460-53483658 | 5:54520466-54521071 | 5:55220508-55221127 |
| 5:55530632-55531090 | 5:55821891-55822291 | 5:56480277-56480744 |
| 5:59038932-59040004 | 5:59892134-59892536 | 5:59892942-59894258 |
| 5:60374292-60374818 | 5:61625106-61626214 | 5:66828547-66828947 |
| 5:67010786-67011452 | 5:68266776-68268522 | 5:71718757-71719180 |
| 5:71719184-71719846 | 5:72108764-72109220 | 5:72556486-72557185 |
| 5:73230401-73230819 | 5:73232133-73234795 | 5:73299647-73300064 |
| 5:73302887-73303826 | 5:73381309-73382516 | 5:73443790-73444783 |
| 5:74865183-74865584 | 5:75461014-75461484 | 5:75611311-75612450 |
| 5:76084961-76085362 | 5:77358711-77359285 | 5:77627967-77628515 |
| 5:77636014-77636413 | 5:77640385-77640961 | 5:77843956-77845422 |
| 5:77852577-77852991 | 5:77957580-77958316 | 5:77972476-77973668 |
| 5:78056752-78057333 | 5:78649204-78649674 | 5:79069327-79070376 |
| 5:79111704-79112674 | 5:80034955-80035804 | 5:81394195-81394596 |
| 5:81852662-81853299 | 5:84383922-84384323 | 5:88144254-88144653 |
| 5:88145113-88145620 | 5:88145903-88146304 | 5:88675886-88676287 |
| 5:88693155-88693555 | 5:93573162-93573563 | 5:93598727-93599283 |
| 5:93602284-93602684 | 5:93603985-93604607 | 5:95619868-95620268 |
| 5:96433057-96433457 | 5:100860171-100861614 | 5:102296316-102296947 |
| 5:112737500-112738222 | 5:113292946-113294494 | 5:114056099-114056628 |
| 5:115178873-115179558 | 5:115816538-115817238 | 5:115962856-115964151 |
| 5:116948291-116948776 | 5:119038381-119038896 | 5:122311308-122311763 |
| 5:123285084-123285745 | 5:124735145-124735564 | 5:126869152-126869553 |
| 5:127080008-127080479 | 5:128538743-128539620 | 5:132257254-132257654 |
| 5:132257863-132260522 | 5:132464477-132465034 | 5:132819607-132820013 |
| 5:133256245-133256724 | 5:134070654-134071055 | 5:134115574-134116180 |
| 5:134527707-134528190 | 5:134545031-134545718 | 5:135031414-135032888 |
| 5:135034670-135035149 | 5:135399743-135400143 | 5:135578530-135579194 |
| 5:135930623-135931023 | 5:138274287-138274909 | 5:139391283-139392493 |
| 5:139481291-139481722 | 5:139482515-139483235 | 5:139696610-139697612 |
| 5:139709538-139710114 | 5:139903126-139903764 | 5:139904514-139905339 |
| 5:140145921-140146792 | 5:140795079-140795478 | 5:140821347-140824087 |
| 5:140827513-140830599 | 5:140834335-140836797 | 5:140855627-140858424 |
| 5:141121668-141122069 | 5:141245150-141245971 | 5:141355040-141355514 |
| 5:141382814-141383215 | 5:141398553-141400432 | 5:141407295-141408447 |
| 5:141417454-141418104 | 5:141421043-141421512 | 5:141421568-141422065 |
| 5:141430271-141433300 | 5:141484302-141485417 | 5:141512589-141514843 |
| 5:141883428-141883827 | 5:142153989-142154435 | 5:142325695-142326124 |
| 5:145856375-145856776 | 5:145856498-145856899 | 5:146334033-146334469 |
| 5:146339079-146340682 | 5:146409263-146409916 | 5:147782429-147782828 |
| 5:149271187-149271794 | 5:149724961-149725413 | 5:149932675-149933090 |
| 5:150155598-150156360 | 5:150412535-150412935 | 5:150413048-150413448 |
| 5:150488372-150489091 | 5:150779826-150782140 | 5:150790070-150790876 |
| 5:151020071-151021284 | 5:151157139-151157635 | 5:151686556-151687320 |
| 5:154850318-154851029 | 5:157459779-157460179 | 5:157919392-157920304 |
| 5:158234351-158234843 | 5:159051372-159051773 | 5:159094728-159097791 |
| 5:159103936-159104990 | 5:160311643-160312059 | 5:167720569-167721389 |
| 5:167770356-167770919 | 5:167814762-167815450 | 5:168114518-168114963 |
| 5:169299646-169300817 | 5:169636916-169637676 | 5:171307878-171309312 |
| 5:171309090-171309718 | 5:171313392-171314083 | 5:171450685-171451392 |
| 5:171807197-171807748 | 5:172111179-172111703 | 5:172174529-172174976 |
| 5:173228687-173229095 | 5:173231898-173232789 | 5:173236928-173237586 |
| 5:173243675-173244076 | 5:173245480-173245881 | 5:173662326-173662826 |
| 5:174751539-174752031 | 5:175772618-175773296 | 5:176175182-176175696 |
| 5:176365807-176366208 | 5:176365920-176366693 | 5:176679917-176680576 |
| 5:176945401-176946363 | 5:177116749-177117197 | 5:177132406-177133265 |
| 5:177985516-177985917 | 5:178477356-178477883 | 5:178576860-178577353 |
| 5:178793054-178793567 | 5:178994559-178995289 | 5:179516374-179517539 |
| 5:179530337-179531103 | 5:179793393-179794240 | 5:179818565-179819048 |
| 5:179819384-179819784 | 5:180590472-180592002 | 5:180648540-180649318 |
| 5:180803482-180804345 | 5:181163957-181165055 | 6:219831-220302 |
| 6:603190-603626 | 6:1378352-1380110 | 6:1384258-1385470 |
| 6:1390821-1391673 | 6:1624334-1625720 | 6:2841083-2842275 |
| 6:2876010-2876410 | 6:2902873-2903620 | 6:2966278-2966756 |
| 6:2981414-2982752 | 6:3231640-3232163 | 6:3752367-3752767 |
| 6:4774537-4775142 | 6:5025834-5026468 | 6:5994766-5995288 |
| 6:6557986-6558468 | 6:6712577-6713008 | 6:6900501-6901222 |
| 6:7106685-7107085 | 6:9398894-9399405 | 6:10415417-10416290 |
| 6:10425926-10426574 | 6:10881398-10884232 | 6:10886551-10886951 |
| 6:13762511-13763146 | 6:15244265-15244866 | 6:16320078-16320478 |
| 6:17102229-17102629 | 6:19691272-19692223 | 6:19836623-19837024 |
| 6:21665418-21665949 | 6:21666172-21668018 | 6:26016901-26018107 |
| 6:26087228-26088368 | 6:26171712-26172319 | 6:26172502-26172903 |
| 6:26188590-26189332 | 6:26240126-26240861 | 6:26249968-26250858 |
| 6:26251836-26252237 | 6:26270773-26272704 | 6:26273117-26273517 |
| 6:27091785-27092221 | 6:27096775-27097374 | 6:27214962-27215363 |
| 6:27267877-27268277 | 6:27495184-27495989 | 6:27545467-27545867 |
| 6:27777217-27777644 | 6:27867444-27867844 | 6:27873299-27873698 |
| 6:27873680-27874079 | 6:27890350-27891009 | 6:28206946-28207818 |
| 6:28208005-28208406 | 6:28399142-28400399 | 6:28474542-28475253 |
| 6:28489284-28490674 | 6:28537451-28537852 | 6:28553826-28554226 |
| 6:28589154-28589979 | 6:28589832-28590233 | 6:28606429-28607444 |
| 6:28635801-28636202 | 6:28729855-28730255 | 6:28816860-28817394 |
| 6:28988290-28988691 | 6:29553799-29554200 | 6:29627295-29627880 |
| 6:29724019-29724418 | 6:29749287-29749687 | 6:29792010-29792410 |
| 6:29792179-29792579 | 6:29827667-29828459 | 6:29828387-29828788 |
| 6:29850294-29850694 | 6:29887127-29888009 | 6:29925890-29927701 |
| 6:29942244-29942644 | 6:29942364-29942784 | 6:29942818-29943219 |
| 6:29943122-29943522 | 6:29975430-29975890 | 6:30006028-30006427 |
| 6:30006138-30007862 | 6:30127052-30127983 | 6:30127750-30128151 |
| 6:30171963-30172791 | 6:30259862-30260262 | 6:30260093-30260493 |
| 6:30490177-30490578 | 6:30743145-30744256 | 6:30743976-30744704 |
| 6:30769510-30770216 | 6:30955297-30955697 | 6:31861366-31864754 |
| 6:32058728-32059170 | 6:32151676-32152076 | 6:32152013-32153806 |
| 6:32188341-32189970 | 6:32223803-32224398 | 6:32255108-32255896 |
| 6:32976421-32978005 | 6:33008316-33009863 | 6:33192160-33192651 |
| 6:33193324-33193963 | 6:33201672-33203212 | 6:33430995-33431482 |
| 6:33854397-33854961 | 6:34235225-34236260 | 6:35213170-35213784 |
| 6:35497917-35498641 | 6:35511700-35512283 | 6:36660757-36661156 |
| 6:36677572-36678021 | 6:36912491-36912963 | 6:37083196-37083729 |
| 6:37565882-37566321 | 6:37782673-37784888 | 6:38715029-38715595 |
| 6:41375570-41376242 | 6:41470174-41471123 | 6:41638428-41639281 |
| 6:41894103-41894728 | 6:42770982-42771772 | 6:42911457-42911857 |
| 6:43243283-43243683 | 6:43263149-43263476 | 6:43308684-43309156 |
| 6:44219105-44219506 | 6:44219338-44219966 | 6:44342340-44342858 |
| 6:44560751-44561570 | 6:45423477-45424802 | 6:48068011-48068553 |
| 6:49550763-49551687 | 6:50823288-50823689 | 6:50840763-50841250 |
| 6:50842568-50843152 | 6:50845530-50846861 | 6:50849261-50849662 |
| 6:50850821-50851673 | 6:52362768-52363376 | 6:54980129-54981263 |
| 6:55578662-55579699 | 6:70282187-70283358 | 6:70957253-70957735 |
| 6:72621235-72621827 | 6:73314472-73315446 | 6:73580106-73581079 |
| 6:77466754-77467239 | 6:78629277-78629678 | 6:78910495-78911233 |
| 6:83853883-83854495 | 6:84772673-84773072 | 6:86473293-86474135 |
| 6:87152289-87152696 | 6:88166871-88167495 | 6:99602891-99603390 |
| 6:99608760-99609417 | 6:99613084-99613581 | 6:99618418-99619444 |
| 6:99994036-99994436 | 6:100448726-100449127 | 6:100455200-100456091 |
| 6:100463638-100464037 | 6:100464849-100465250 | 6:100466925-100467326 |
| 6:100467164-100467669 | 6:100469336-100469735 | 6:104940129-104941024 |
| 6:104952858-104953268 | 6:104953579-104953979 | 6:105136107-105136507 |
| 6:105302477-105302996 | 6:105981290-105981718 | 6:107488574-107489108 |
| 6:108118763-108119549 | 6:108158137-108158538 | 6:108163668-108164068 |
| 6:108165666-108166066 | 6:108165983-108166384 | 6:108166465-108167282 |
| 6:108168440-108170648 | 6:108174323-108175406 | 6:108176321-108176721 |
| 6:111113652-111114450 | 6:111566354-111567818 | 6:116370068-116370850 |
| 6:116764726-116765466 | 6:116765536-116766043 | 6:117481354-117481754 |
| 6:121437356-121438826 | 6:122996183-122996582 | 6:127514694-127515164 |
| 6:128882829-128883515 | 6:133240219-133243035 | 6:133888966-133890127 |
| 6:134539866-134540625 | 6:136920634-136921328 | 6:137045034-137045505 |
| 6:137487836-137488643 | 6:137492590-137494346 | 6:137496346-137496788 |
| 6:138455812-138456799 | 6:139473199-139474604 | 6:142702453-142703042 |
| 6:142885029-142885839 | 6:142926454-142926853 | 6:145814951-145815763 |
| 6:146434014-146434914 | 6:148508446-148509919 | 6:148760681-148761785 |
| 6:149964870-149965660 | 6:150042026-150043151 | 6:150142212-150142789 |
| 6:151808772-151809418 | 6:152473736-152474137 | 6:154085378-154085856 |
| 6:155411667-155412210 | 6:157763566-157764856 | 6:157982858-157983259 |
| 6:158242254-158242714 | 6:159107357-159107998 | 6:159953029-159953589 |
| 6:160678753-160679805 | 6:165804827-165805227 | 6:166008543-166009003 |
| 6:166898615-166899086 | 6:167677941-167678897 | 6:168318762-168319629 |
| 7:214426-214835 | 7:266934-267489 | 7:529799-530648 |
| 7:590793-591427 | 7:635914-636804 | 7:711194-712280 |
| 7:767872-769096 | 7:830622-832602 | 7:881286-881687 |
| 7:907006-908064 | 7:936994-937732 | 7:1054803-1056712 |
| 7:1177079-1177479 | 7:1223231-1224491 | 7:1230771-1231439 |
| 7:1244102-1244502 | 7:1246236-1246663 | 7:1253882-1254527 |
| 7:1733370-1733771 | 7:1810416-1811315 | 7:2193277-2193677 |
| 7:2333614-2334058 | 7:2508251-2509423 | 7:2688879-2689538 |
| 7:2757653-2758697 | 7:2956673-2957197 | 7:3043501-3043901 |
| 7:4144368-4144872 | 7:4845739-4846162 | 7:5072134-5072586 |
| 7:5237156-5237951 | 7:6664021-6664593 | 7:8440958-8444425 |
| 7:12571210-12571945 | 7:18495335-18496427 | 7:19106258-19106782 |
| 7:19106667-19107082 | 7:19112243-19113013 | 7:19118575-19119481 |
| 7:19143789-19145787 | 7:19773448-19773848 | 7:20304541-20305138 |
| 7:20793727-20794127 | 7:20798733-20799132 | 7:21169395-21170312 |
| 7:22577398-22578388 | 7:22725550-22726257 | 7:23468387-23468787 |
| 7:24284800-24285507 | 7:24923407-24924643 | 7:24978672-24979255 |
| 7:25860396-25860885 | 7:25952490-25953250 | 7:26363971-26364370 |
| 7:27122180-27122983 | 7:27156370-27157086 | 7:27202274-27202692 |
| 7:27203506-27203994 | 7:27204600-27205095 | 7:27242087-27242487 |
| 7:28296785-28298287 | 7:28623955-28624597 | 7:28958426-28959244 |
| 7:29146198-29146881 | 7:29231048-29231598 | 7:29565027-29566886 |
| 7:29988478-29988877 | 7:29989930-29990330 | 7:30593691-30594174 |
| 7:30682507-30682907 | 7:31102717-31103274 | 7:31107515-31107915 |
| 7:31478938-31479503 | 7:31488564-31489039 | 7:32298122-32299213 |
| 7:32427782-32428868 | 7:32941981-32942554 | 7:34991847-34992600 |
| 7:35253945-35254344 | 7:35454584-35455411 | 7:37191693-37192263 |
| 7:38266071-38266542 | 7:38311169-38311643 | 7:39413865-39414265 |
| 7:39609474-39610257 | 7:39854833-39855362 | 7:42238057-42238834 |
| 7:43648605-43649173 | 7:44145573-44146537 | 7:44276040-44276547 |
| 7:44915019-44915388 | 7:45026902-45027609 | 7:45088232-45088726 |
| 7:45157690-45158160 | 7:45573599-45574276 | 7:45663333-45663753 |
| 7:46752980-46753611 | 7:47475012-47475772 | 7:47520447-47520980 |
| 7:47991811-47992353 | 7:49773286-49775945 | 7:49788014-49788565 |
| 7:50304080-50304479 | 7:50304547-50305025 | 7:50362588-50364040 |
| 7:50401838-50402570 | 7:51471073-51471967 | 7:52088323-52088943 |
| 7:53219077-53219583 | 7:54203308-54203739 | 7:54544401-54545174 |
| 7:54620441-54620936 | 7:54888315-54889100 | 7:56174391-56175175 |
| 7:64494853-64495640 | 7:64569440-64569841 | 7:65269587-65269987 |
| 7:66043671-66044288 | 7:72335513-72336108 | 7:73424120-73424597 |
| 7:73587904-73588316 | 7:74480249-74481098 | 7:75482669-75483190 |
| 7:75905972-75906373 | 7:76364196-76365043 | 7:77327020-77328012 |
| 7:79453433-79454684 | 7:82822379-82823418 | 7:85041841-85042416 |
| 7:87474931-87475602 | 7:87600729-87601178 | 7:90215369-90216850 |
| 7:95324309-95325041 | 7:95396148-95396959 | 7:95916995-95917587 |
| 7:96996036-96996545 | 7:96996688-96997497 | 7:97002831-97003679 |
| 7:97022759-97023302 | 7:97026422-97026823 | 7:97731152-97732597 |
| 7:98210407-98210951 | 7:98215416-98215816 | 7:98470420-98470821 |
| 7:98826389-98826884 | 7:99140870-99142549 | 7:99557899-99558419 |
| 7:99579801-99580286 | 7:99997502-99997985 | 7:100171658-100172129 |
| 7:100493407-100494313 | 7:100720383-100720783 | 7:100865433-100866036 |
| 7:100866998-100868895 | 7:100890755-100891551 | 7:101126841-101127486 |
| 7:102045209-102045608 | 7:102366922-102367549 | 7:104944127-104944744 |
| 7:105678960-105679382 | 7:105877092-105877773 | 7:107742550-107743215 |
| 7:108001803-108002203 | 7:116499923-116500689 | 7:116524219-116524835 |
| 7:117479219-117480160 | 7:118214099-118214861 | 7:120856569-120857232 |
| 7:120988669-120989147 | 7:121328714-121329175 | 7:122299622-122300440 |
| 7:122304027-122304428 | 7:122306402-122307055 | 7:122316438-122316986 |
| 7:122316999-122317400 | 7:126667628-126668161 | 7:127536650-127537051 |
| 7:128030813-128031292 | 7:128031948-128032755 | 7:128168644-128169106 |
| 7:128271713-128272114 | 7:128360566-128360966 | 7:128697278-128697821 |
| 7:128790384-128790918 | 7:128830658-128831059 | 7:128910626-128911026 |
| 7:129134443-129134817 | 7:129169108-129169537 | 7:129169309-129169751 |
| 7:129781312-129781713 | 7:129781685-129786324 | 7:130913096-130913594 |
| 7:131557877-131558353 | 7:134458078-134459687 | 7:136871160-136871730 |
| 7:137343268-137344021 | 7:138257445-138258234 | 7:138797930-138798548 |
| 7:139110076-139110476 | 7:139523954-139524383 | 7:140229904-140230606 |
| 7:140527234-140527633 | 7:141053584-141053991 | 7:141511676-141512306 |
| 7:142856528-142857223 | 7:143288665-143289751 | 7:143345341-143345855 |
| 7:143382028-143382846 | 7:143882346-143883346 | 7:143884884-143885735 |
| 7:148697548-148698239 | 7:149414900-149415462 | 7:149496164-149496744 |
| 7:149692427-149693000 | 7:149715245-149715913 | 7:150219318-150220324 |
| 7:150339974-150342092 | 7:150357300-150357650 | 7:150799822-150800223 |
| 7:150800823-150802143 | 7:150955463-150956319 | 7:150957812-150958580 |
| 7:151013113-151014597 | 7:151051243-151051648 | 7:151247362-151248000 |
| 7:151410048-151410988 | 7:151782607-151783008 | 7:152029920-152030739 |
| 7:152924781-152926450 | 7:153748970-153749371 | 7:153886084-153889357 |
| 7:154051173-154053609 | 7:155372099-155372500 | 7:155373444-155374009 |
| 7:155459805-155460206 | 7:155462276-155462765 | 7:155465467-155465868 |
| 7:155466999-155467929 | 7:155468049-155468489 | 7:155809031-155809516 |
| 7:157002034-157003223 | 7:157003069-157006266 | 7:157020096-157020641 |
| 7:157020635-157021058 | 7:157021635-157022486 | 7:157077977-157078693 |
| 7:157260150-157260550 | 7:157568197-157569552 | 7:157682822-157683222 |
| 7:157683486-157689966 | 7:157691202-157692923 | 7:157693875-157694524 |
| 7:157875084-157875600 | 7:157904545-157906002 | 7:158026981-158027561 |
| 7:158468252-158468653 | 7:158473275-158473674 | 7:158569424-158570604 |
| 7:158588182-158588659 | 7:158959558-158960175 | 7:159008010-159008556 |
| 7:159143789-159144197 | 7:159145410-159145999 | 8:739159-739558 |
| 8:1097923-1098323 | 8:1946642-1947042 | 8:2006343-2007131 |
| 8:3396257-3396656 | 8:6780145-6780712 | 8:10728357-10729695 |
| 8:11014943-11015459 | 8:11702842-11703242 | 8:11703933-11704796 |
| 8:15405835-15406377 | 8:17412792-17413249 | 8:17413174-17413746 |
| 8:17661706-17662232 | 8:18387150-18387551 | 8:19602010-19602775 |
| 8:20270185-20271279 | 8:22597168-22597607 | 8:22600382-22601043 |
| 8:22703308-22703786 | 8:23102721-23103725 | 8:23335936-23336483 |
| 8:23402685-23403390 | 8:23571262-23571928 | 8:23702324-23703227 |
| 8:23705254-23705819 | 8:23706195-23706607 | 8:23706361-23707228 |
| 8:23709646-23710384 | 8:23726434-23727397 | 8:24293623-24294279 |
| 8:24913450-24914103 | 8:26039534-26042093 | 8:26049501-26049902 |
| 8:26515138-26515537 | 8:27334579-27335018 | 8:27611273-27611674 |
| 8:28189938-28190526 | 8:30385534-30385934 | 8:31639299-31640890 |
| 8:32594847-32596113 | 8:37393169-37393715 | 8:37479310-37479724 |
| 8:38465546-38465946 | 8:38553929-38554340 | 8:38666392-38666909 |
| 8:38727933-38728698 | 8:38937035-38937942 | 8:39107323-39108017 |
| 8:41653318-41653945 | 8:41798004-41798526 | 8:42207577-42208278 |
| 8:42265454-42266017 | 8:42498968-42499503 | 8:47591678-47592399 |
| 8:47889200-47889680 | 8:48554914-48556811 | 8:48870509-48871049 |
| 8:52565743-52566296 | 8:53876496-53877568 | 8:53879052-53879752 |
| 8:53944986-53945456 | 8:54453495-54455057 | 8:54458240-54458959 |
| 8:55919574-55920088 | 8:55939136-55940288 | 8:56445419-56447740 |
| 8:56477320-56478225 | 8:57343560-57344011 | 8:58145416-58146251 |
| 8:59117092-59118384 | 8:60908736-60910407 | 8:60922001-60923439 |
| 8:61201559-61202924 | 8:62247998-62249655 | 8:64368759-64370344 |
| 8:64377612-64378077 | 8:64585592-64587520 | 8:64798814-64799509 |
| 8:66432135-66432535 | 8:66432256-66432921 | 8:66960957-66962093 |
| 8:66962647-66963356 | 8:67745851-67746481 | 8:67746182-67746890 |
| 8:69562541-69563304 | 8:70034392-70035510 | 8:70071146-70071656 |
| 8:70071813-70072474 | 8:71547053-71548490 | 8:71556177-71557338 |
| 8:71841425-71844626 | 8:71922875-71924332 | 8:72251432-72251833 |
| 8:72537138-72538096 | 8:73092366-73092843 | 8:75404604-75405005 |
| 8:76682821-76684121 | 8:77565582-77566075 | 8:79610741-79612277 |
| 8:84184608-84185008 | 8:85438001-85439117 | 8:86069390-86069899 |
| 8:86657838-86658461 | 8:90095809-90096542 | 8:90984621-90985153 |
| 8:90985431-90985907 | 8:92101450-92102359 | 8:93699739-93700295 |
| 8:93700769-93701672 | 8:95693307-95693889 | 8:96159448-96159934 |
| 8:96645390-96645789 | 8:97776302-97776990 | 8:98042537-98044084 |
| 8:98942053-98942479 | 8:98972141-98974923 | 8:100105704-100106470 |
| 8:101108558-101110020 | 8:101125912-101126443 | 8:101223887-101224800 |
| 8:102789110-102789599 | 8:102791022-102791469 | 8:103141189-103141589 |
| 8:104466201-104466980 | 8:104466855-104467254 | 8:105798479-105799065 |
| 8:107497886-107498286 | 8:108083541-108084224 | 8:109973602-109976028 |
| 8:112690327-112691097 | 8:116448139-116448626 | 8:116937854-116938307 |
| 8:118108841-118109242 | 8:118110040-118110789 | 8:119638603-119639562 |
| 8:120125489-120126143 | 8:121261468-121261869 | 8:121322817-121324300 |
| 8:121639090-121639490 | 8:123160840-123161295 | 8:123182169-123183044 |
| 8:124247217-124247849 | 8:124709226-124709908 | 8:125388268-125389066 |
| 8:125435640-125436745 | 8:128176608-128177104 | 8:129628788-129629253 |
| 8:130045752-130046274 | 8:130442852-130443287 | 8:131039978-131042829 |
| 8:132057814-132058312 | 8:133321887-133322396 | 8:133375549-133376071 |
| 8:134457042-134457588 | 8:139702095-139702517 | 8:139703146-139703709 |
| 8:141237720-141239314 | 8:141273167-141273708 | 8:141291825-141292312 |
| 8:141376103-141376638 | 8:141507157-141507556 | 8:141760377-141760778 |
| 8:141905098-141905498 | 8:142251975-142252568 | 8:142699728-142700138 |
| 8:142710254-142711013 | 8:142726044-142726522 | 8:143599102-143599930 |
| 8:143716310-143717002 | 8:143770552-143771209 | 8:143948129-143949119 |
| 8:143950520-143951743 | 8:144008088-144008753 | 8:144048341-144049608 |
| 8:144274649-144275752 | 8:144472698-144473427 | 9:503685-504145 |
| 9:676847-678025 | 9:971329-971907 | 9:1042051-1043019 |
| 9:4858969-4859484 | 9:6644218-6644753 | 9:14156062-14156595 |
| 9:14346604-14349640 | 9:14350576-14351093 | 9:18437830-18439096 |
| 9:19788108-19788716 | 9:20552282-20552767 | 9:21966286-21968879 |
| 9:21970766-21971610 | 9:21988472-21990259 | 9:22005143-22006157 |
| 9:27528071-27530059 | 9:32783126-32783831 | 9:32954641-32957000 |
| 9:33167871-33168380 | 9:33448027-33448698 | 9:33750677-33751529 |
| 9:34381381-34382170 | 9:35490324-35491875 | 9:35791745-35792405 |
| 9:35907970-35908460 | 9:36470747-36471292 | 9:36985808-36986209 |
| 9:37002487-37002888 | 9:37024065-37025683 | 9:37029657-37030415 |
| 9:37031907-37032498 | 9:37035033-37036965 | 9:62801578-62803531 |
| 9:68357341-68359262 | 9:69173572-69174978 | 9:70421359-70422215 |
| 9:71984864-71985274 | 9:75892285-75892767 | 9:76447708-76448338 |
| 9:76665695-76666365 | 9:76692027-76692753 | 9:77012099-77012500 |
| 9:77014601-77015539 | 9:77016670-77017459 | 9:84102322-84102723 |
| 9:84670469-84671066 | 9:84711604-84712482 | 9:85522831-85523231 |
| 9:86079153-86079761 | 9:86096361-86098696 | 9:87909553-87910130 |
| 9:88990827-88991234 | 9:89234594-89234995 | 9:90000862-90001349 |
| 9:90433305-90433982 | 9:92147245-92148005 | 9:93004098-93004533 |
| 9:93098398-93098999 | 9:93120019-93120523 | 9:93345984-93346385 |
| 9:93860991-93861503 | 9:93947336-93949643 | 9:93952614-93953555 |
| 9:93959035-93959685 | 9:93960569-93960968 | 9:94554742-94555262 |
| 9:95348934-95350299 | 9:96021798-96022415 | 9:96719611-96720012 |
| 9:97502424-97503239 | 9:97950884-97951333 | 9:99824809-99825906 |
| 9:99828261-99829171 | 9:100474116-100474564 | 9:107425174-107425575 |
| 9:110047921-110048322 | 9:110048227-110048674 | 9:111038478-111039430 |
| 9:112749952-112750560 | 9:113687684-113688412 | 9:114049712-114050259 |
| 9:117744744-117745549 | 9:120928094-120928792 | 9:121581667-121582105 |
| 9:121700318-121702766 | 9:121736203-121736832 | 9:122213239-122213720 |
| 9:122219502-122220826 | 9:122228913-122229445 | 9:124013700-124014101 |
| 9:124032322-124032721 | 9:124280199-124280672 | 9:124282643-124283365 |
| 9:124777626-124779215 | 9:124800404-124801186 | 9:124832577-124833081 |
| 9:126375493-126376672 | 9:126624787-126625188 | 9:126719411-126720228 |
| 9:127121883-127122328 | 9:127122518-127122951 | 9:127926775-127927265 |
| 9:127971176-127972009 | 9:128365043-128365424 | 9:128587392-128587847 |
| 9:128902287-128902770 | 9:128911514-128912859 | 9:128922443-128922844 |
| 9:129665126-129665525 | 9:130445190-130446077 | 9:130658999-130659574 |
| 9:130740372-130740876 | 9:131361157-131361643 | 9:131667954-131669394 |
| 9:131732928-131734207 | 9:132579413-132580066 | 9:132586397-132586932 |
| 9:132589276-132589793 | 9:133059605-133060214 | 9:133060942-133061710 |
| 9:133162336-133163560 | 9:133349220-133350649 | 9:133429511-133430101 |
| 9:134223057-134223540 | 9:134376027-134376428 | 9:134842600-134845065 |
| 9:135088824-135089262 | 9:135109101-135109551 | 9:135247384-135247930 |
| 9:135480850-135481300 | 9:136050192-136051258 | 9:136081087-136081635 |
| 9:136110046-136110447 | 9:136342033-136342830 | 9:136342830-136343229 |
| 9:136550388-136551820 | 9:136686196-136686721 | 9:136942678-136943590 |
| 9:137022150-137022661 | 9:137139285-137140607 | 9:137155918-137156330 |
| 9:137187291-137187692 | 9:137196590-137197221 | 9:137198894-137199510 |
| 9:137218338-137219067 | 9:137278028-137278606 | 9:137302723-137303589 |
| X:2927954-2928355 | X:3269557-3270119 | X:3309209-3309610 |
| X:3345457-3347513 | X:8730221-8730647 | X:8742822-8743355 |
| X:9339868-9341564 | X:9464303-9464702 | X:9765096-9765655 |
| X:11787403-11787935 | X:12955891-12956615 | X:13569280-13570333 |
| X:17377174-17378018 | X:20116076-20116752 | X:20116587-20116988 |
| X:20267974-20268645 | X:21658075-21658903 | X:21658733-21659134 |
| X:22686150-22686582 | X:23332045-23332684 | X:24646806-24647329 |
| X:24647401-24647965 | X:25011017-25011905 | X:25012058-25012736 |
| X:25015499-25016658 | X:25017549-25017950 | X:25022639-25023291 |
| X:36957806-36958207 | X:38220892-38221292 | X:40091815-40092537 |
| X:40099754-40101263 | X:43882119-43882519 | X:47573824-47574673 |
| X:47582023-47582530 | X:47623434-47623834 | X:49165920-49167137 |
| X:49230621-49231073 | X:50469173-50469816 | X:50813677-50814860 |
| X:50905207-50905805 | X:51333292-51334432 | X:51395787-51396364 |
| X:51396244-51396779 | X:52868448-52869245 | X:54358500-54359515 |
| X:54920724-54921539 | X:56231803-56233311 | X:63784853-63785636 |
| X:65668370-65668769 | X:69128471-69128904 | X:69305619-69306190 |
| X:70422840-70423337 | X:70930721-70931509 | X:71103742-71104212 |
| X:73447085-73448922 | X:74421629-74423200 | X:76428417-76429407 |
| X:83508034-83508434 | X:83510384-83510785 | X:100407783-100408225 |
| X:101078810-101079287 | X:101191102-101191898 | X:101550369-101550850 |
| X:102650758-102652908 | X:102711922-102712323 | X:102745549-102747158 |
| X:103253724-103254125 | X:104013203-104013703 | X:104013504-104013905 |
| X:104255754-104256155 | X:105822648-105823468 | X:107272562-107273095 |
| X:107506364-107507781 | X:108438016-108438745 | X:108732760-108733160 |
| X:109624857-109625386 | X:115233882-115234466 | X:115958105-115958886 |
| X:118494999-118495400 | X:118973425-118974063 | X:119235243-119235769 |
| X:119273182-119274039 | X:119990101-119990696 | X:119999586-120000154 |
| X:120000215-120001267 | X:120014701-120015379 | X:120308791-120309572 |
| X:120310832-120312160 | X:123183610-123184815 | X:123784764-123785327 |
| X:125205930-125206699 | X:126552617-126553018 | X:129523569-129524066 |
| X:129953310-129953711 | X:130108936-130110048 | X:130111059-130111460 |
| X:130120678-130121313 | X:130170744-130171213 | X:131057653-131058053 |
| X:131058009-131058751 | X:131082255-131083207 | X:132025041-132025776 |
| X:132958341-132958741 | X:133413208-133413690 | X:133417185-133417908 |
| X:134540259-134540916 | X:134544535-134545158 | X:134549996-134551445 |
| X:134990658-134991379 | X:135295578-135296311 | X:135520161-135520562 |
| X:135888977-135890033 | X:135974550-135974989 | X:137031295-137031716 |
| X:137550222-137550995 | X:137565921-137566322 | X:137573900-137575076 |
| X:138710501-138711284 | X:138711507-138712203 | X:139642298-139642698 |
| X:139691929-139693067 | X:140504976-140505376 | X:140510817-140511567 |
| X:143633228-143634243 | X:143635487-143636157 | X:148718223-148719062 |
| X:150365671-150366141 | X:151176943-151177344 | X:151695292-151695693 |
| X:151697929-151698839 | X:151957487-151958946 | X:151974218-151975257 |
| X:152897476-152897877 | X:153676040-153676611 | X:153828529-153829662 |
| X:154370223-154370684 | X:155612484-155613330 | Y:2787369-2788051 |
| Y:7272380-7272828 | Y:7275702-7276436 | Y:7279419-7280208 |

### Table 4A—PrCa

The DMRs may be those disclosed in Table 4 of GB2215319.1 and which are also available at: https://doi.org/10.5281/zenodo.10001275 as of 17 Oct. 2023. The entire contents of Table 4 of GB2215319.1 and https://doi.org/10.5281/zenodo.10001275 are expressly incorporated herein by reference as “Table 4A”.

TABLE 6
PrCa (CaNCaP)

| | | |
|---|---|---|
| 1: 823296-823716 | 1: 1060647-1061067 | 1: 1144009-1144487 |
| 1: 1233399-1233838 | 1: 1304838-1305273 | 1: 1347093-1347513 |
| 1: 1559153-1559573 | 1: 1598949-1599539 | 1: 1630176-1630698 |
| 1: 1788886-1789306 | 1: 1980423-1980897 | 1: 2018336-2018756 |
| 1: 2043233-2043653 | 1: 2134798-2135218 | 1: 2248327-2248747 |
| 1: 2403937-2404357 | 1: 2408314-2408983 | 1: 2409896-2410338 |
| 1: 2410420-2410840 | 1: 2787638-2788039 | 1: 2800108-2800528 |
| 1: 2826775-2827195 | 1: 2852710-2853189 | 1: 2857564-2858018 |
| 1: 2858603-2859023 | 1: 2907042-2907462 | 1: 2934065-2934485 |
| 1: 2961678-2962098 | 1: 3001783-3002247 | 1: 3022172-3022742 |
| 1: 3030234-3030654 | 1: 3080689-3081109 | 1: 3084222-3084642 |
| 1: 3084435-3084855 | 1: 3090950-3091470 | 1: 3163971-3164391 |
| 1: 3195016-3195436 | 1: 3241155-3241575 | 1: 3243933-3244353 |
| 1: 3262972-3263434 | 1: 3267944-3268364 | 1: 3278663-3279102 |
| 1: 3278965-3279380 | 1: 3285144-3285637 | 1: 3289557-3289977 |
| 1: 3290558-3290961 | 1: 3390451-3390871 | 1: 3411278-3411884 |
| 1: 3411973-3412575 | 1: 3414501-3414923 | 1: 3417328-3417697 |
| 1: 3425474-3425894 | 1: 3458192-3458726 | 1: 3587376-3587763 |
| 1: 3718095-3718515 | 1: 3771793-3772213 | 1: 3892820-3893240 |
| 1: 3923276-3923696 | 1: 3976986-3977406 | 1: 4018877-4019297 |
| 1: 4059369-4059752 | 1: 4132537-4132957 | 1: 4411856-4412277 |
| 1: 4425375-4425795 | 1: 4463713-4464161 | 1: 4571132-4571606 |
| 1: 4662418-4662838 | 1: 4768347-4768767 | 1: 4772220-4772640 |
| 1: 4772371-4772791 | 1: 5971836-5972256 | 1: 6208660-6209091 |
| 1: 6209185-6209605 | 1: 6462278-6462698 | 1: 6743590-6744011 |
| 1: 6752188-6752608 | 1: 6962041-6962494 | 1: 6998457-6998877 |
| 1: 7219001-7219421 | 1: 7234269-7234689 | 1: 8154590-8155043 |
| 1: 8197689-8198160 | 1: 8211864-8212474 | 1: 8364575-8364995 |
| 1: 9111186-9111606 | 1: 9338904-9339295 | 1: 9505293-9505644 |
| 1: 9553530-9553927 | 1: 9878268-9878688 | 1: 10056507-10056875 |
| 1: 10066990-10067410 | 1: 10530389-10530843 | 1: 10652759-10653161 |
| 1: 10655591-10656011 | 1: 10958589-10959009 | 1: 11013593-11014041 |
| 1: 11058704-11059125 | 1: 12063815-12064235 | 1: 12188608-12189044 |
| 1: 12229289-12229709 | 1: 12636070-12636490 | 1: 13589290-13589710 |
| 1: 13659278-13659698 | 1: 13699488-13699904 | 1: 13730627-13731047 |
| 1: 13730868-13731288 | 1: 15141757-15142177 | 1: 15837308-15837728 |
| 1: 16141603-16142084 | 1: 16316038-16316458 | 1: 16520881-16521301 |
| 1: 16608547-16608962 | 1: 16872902-16873322 | 1: 17163954-17164374 |
| 1: 17318395-17318815 | 1: 17712347-17712767 | 1: 17881647-17882067 |
| 1: 18178031-18178493 | 1: 18385432-18385855 | 1: 18926480-18926926 |
| 1: 18949648-18950068 | 1: 19154829-19155284 | 1: 19273720-19274598 |
| 1: 19650682-19651102 | 1: 19665524-19666296 | 1: 20400016-20400436 |
| 1: 20731363-20731783 | 1: 21468526-21468946 | 1: 21647789-21648319 |
| 1: 22717425-22717845 | 1: 22953684-22954160 | 1: 23563922-23564294 |
| 1: 23949126-23949546 | 1: 24066564-24066984 | 1: 24343466-24343886 |
| 1: 24727456-24727876 | 1: 25022354-25022774 | 1: 25245036-25245449 |
| 1: 26097282-26097673 | 1: 26114181-26114585 | 1: 26224893-26225366 |
| 1: 26345251-26345671 | 1: 26360570-26360990 | 1: 26364551-26364955 |
| 1: 26948293-26948708 | 1: 27012393-27012813 | 1: 27235830-27236250 |
| 1: 27360691-27361111 | 1: 27525290-27525710 | 1: 28869159-28869579 |
| 1: 29121092-29121512 | 1: 29349898-29350318 | 1: 30229995-30230415 |
| 1: 30902123-30902543 | 1: 31703733-31704153 | 1: 31944349-31944769 |
| 1: 32201376-32201796 | 1: 32215841-32216185 | 1: 32223886-32224376 |
| 1: 32322537-32322957 | 1: 32885897-32886317 | 1: 32925806-32926382 |
| 1: 33064339-33064759 | 1: 33306466-33307525 | 1: 34900283-34900684 |
| 1: 34904165-34904557 | 1: 35573526-35573946 | 1: 36320760-36321244 |
| 1: 36321088-36321617 | 1: 36482051-36482471 | 1: 36692359-36692717 |
| 1: 36805956-36806376 | 1: 37475477-37475976 | 1: 37556909-37557329 |
| 1: 38047664-38048148 | 1: 38134288-38134708 | 1: 38247936-38248356 |
| 1: 39204458-39204878 | 1: 39692237-39692690 | 1: 40818152-40818853 |
| 1: 40883925-40884345 | 1: 41809680-41810100 | 1: 41880338-41880758 |
| 1: 41891349-41891814 | 1: 42198175-42198534 | 1: 42757298-42757718 |
| 1: 43565455-43565875 | 1: 43933835-43934255 | 1: 43935535-43936010 |
| 1: 44107429-44107849 | 1: 44238251-44238692 | 1: 44777525-44777944 |
| 1: 44800698-44801238 | 1: 44808226-44808649 | 1: 46187551-46187970 |
| 1: 46547338-46547758 | 1: 46749902-46750322 | 1: 47439423-47439843 |
| 1: 47446072-47446492 | 1: 47781251-47781671 | 1: 50326712-50327132 |
| 1: 50417507-50417927 | 1: 50421536-50421956 | 1: 52553831-52554302 |
| 1: 53062718-53063422 | 1: 53392328-53392748 | 1: 54302838-54303258 |
| 1: 54475407-54475854 | 1: 54485581-54486001 | 1: 54514122-54514501 |
| 1: 54547516-54547936 | 1: 56577854-56578307 | 1: 58584514-58584934 |
| 1: 58755229-58755649 | 1: 59084183-59084603 | 1: 62194827-62195415 |
| 1: 63122205-63122625 | 1: 63318148-63318611 | 1: 63320903-63321323 |
| 1: 63788873-63789301 | 1: 64042335-64042720 | 1: 64140013-64140396 |
| 1: 64268628-64269037 | 1: 64675291-64675711 | 1: 67134560-67135001 |
| 1: 67424951-67425371 | 1: 67736107-67736527 | 1: 71046392-71047063 |
| 1: 73105903-73106323 | 1: 74589564-74589984 | 1: 82775235-82775693 |
| 1: 84297505-84298020 | 1: 84998908-84999328 | 1: 85126367-85126752 |
| 1: 85244725-85245145 | 1: 85277510-85278011 | 1: 85582638-85583058 |
| 1: 86154794-86155267 | 1: 88800416-88800814 | 1: 89632139-89632709 |
| 1: 90724843-90725263 | 1: 90726922-90727342 | 1: 92476223-92476643 |
| 1: 94327236-94327685 | 1: 95793818-95794160 | 1: 99526888-99527279 |
| 1: 101830549-101830869 | 1: 104263047-104263467 | 1: 108560840-108561260 |
| 1: 109543597-109544009 | 1: 110129929-110130349 | 1: 110211576-110212094 |
| 1: 111270727-111271368 | 1: 111516084-111516504 | 1: 112509385-112509805 |
| 1: 112722917-112723337 | 1: 113982590-113983010 | 1: 114506977-114507428 |
| 1: 115707194-115707589 | 1: 116122973-116123383 | 1: 116666380-116666800 |
| 1: 118980000-118980500 | 1: 118983395-118983941 | 1: 118993164-118993746 |
| 1: 118997152-118997572 | 1: 119625736-119626093 | 1: 119764700-119765120 |
| 1: 120954408-120954799 | 1: 125119826-125120246 | 1: 145156646-145157363 |
| 1: 146486514-146486934 | 1: 146943321-146943740 | 1: 148274599-148275019 |
| 1: 148296474-148296838 | 1: 149906973-149907393 | 1: 149913490-149913874 |
| 1: 150533105-150533525 | 1: 150968193-150968613 | 1: 150968424-150968844 |
| 1: 151130897-151131317 | 1: 151717240-151717660 | 1: 152219089-152219509 |
| 1: 152579481-152579902 | 1: 152760314-152760734 | 1: 152760767-152761187 |
| 1: 152870255-152870675 | 1: 153099443-153099945 | 1: 153291642-153292045 |
| 1: 153331152-153331490 | 1: 153397195-153397598 | 1: 153404944-153405308 |
| 1: 153439619-153439963 | 1: 153698152-153698608 | 1: 154502409-154502829 |
| 1: 155062359-155062817 | 1: 155166812-155167232 | 1: 155177359-155177741 |
| 1: 155207935-155208355 | 1: 155321020-155321565 | 1: 155671885-155672239 |
| 1: 155974714-155975312 | 1: 156076772-156077192 | 1: 156335714-156336108 |
| 1: 156410747-156411167 | 1: 156430426-156430846 | 1: 156438866-156439286 |
| 1: 156502805-156503225 | 1: 156503147-156503567 | 1: 156642411-156642887 |
| 1: 156646645-156647065 | 1: 156646830-156647250 | 1: 156848810-156849230 |
| 1: 157168737-157169157 | 1: 157289467-157289874 | 1: 157490275-157490695 |
| 1: 157708822-157709264 | 1: 157798206-157798615 | 1: 157801891-157802241 |
| 1: 157925489-157925928 | 1: 157925767-157926187 | 1: 157925960-157926380 |
| 1: 158016863-158017275 | 1: 158024877-158025297 | 1: 158152964-158153327 |
| 1: 158176950-158177340 | 1: 158195525-158195945 | 1: 158332454-158332762 |
| 1: 158635762-158636118 | 1: 158642559-158642979 | 1: 158718919-158719292 |
| 1: 159024720-159025140 | 1: 159025052-159025472 | 1: 159066317-159066802 |
| 1: 159122408-159122836 | 1: 159250841-159251262 | 1: 159266083-159266461 |
| 1: 159587573-159587993 | 1: 160252677-160253083 | 1: 160691608-160692028 |
| 1: 160692167-160692573 | 1: 161684811-161685231 | 1: 162564657-162565104 |
| 1: 165838117-165838511 | 1: 166588547-166588949 | 1: 166602209-166602613 |
| 1: 167125656-167126096 | 1: 167683318-167683738 | 1: 167713531-167713951 |
| 1: 167926485-167926897 | 1: 168495562-168495992 | 1: 168766530-168766911 |
| 1: 170588765-170589142 | 1: 170662021-170662441 | 1: 171670802-171671222 |
| 1: 173489191-173489600 | 1: 175044655-175045075 | 1: 175082554-175082926 |
| 1: 178371434-178371854 | 1: 178486936-178487401 | 1: 180239476-180239896 |
| 1: 180863579-180863999 | 1: 181104388-181104834 | 1: 181482024-181482444 |
| 1: 181798487-181798907 | 1: 182056640-182057060 | 1: 183185652-183186072 |
| 1: 184258787-184259140 | 1: 184755749-184756224 | 1: 185696272-185696744 |
| 1: 185737828-185738232 | 1: 186089924-186090323 | 1: 186112632-186112978 |
| 1: 186178323-186178743 | 1: 192327976-192328386 | 1: 195897140-195897530 |
| 1: 196678662-196679082 | 1: 197908506-197908968 | 1: 197910019-197910572 |
| 1: 197921127-197921778 | 1: 198893994-198894414 | 1: 198983199-198983702 |
| 1: 199073675-199073992 | 1: 199085217-199085619 | 1: 199271737-199272056 |
| 1: 200038245-200038789 | 1: 200041348-200041994 | 1: 201003544-201003916 |
| 1: 201191866-201192286 | 1: 201368143-201368563 | 1: 201969522-201969942 |
| 1: 202311917-202312337 | 1: 202644731-202645154 | 1: 203289436-203290061 |
| 1: 203290122-203290551 | 1: 203625748-203626168 | 1: 203631618-203632038 |
| 1: 204104527-204104947 | 1: 204340123-204340543 | 1: 204889731-204890151 |
| 1: 204988529-204988949 | 1: 205313582-205314002 | 1: 205492990-205493410 |
| 1: 205528725-205529145 | 1: 205662584-205663032 | 1: 207049120-207049540 |
| 1: 207669077-207669497 | 1: 207959285-207959705 | 1: 208697288-208697797 |
| 1: 209231422-209231842 | 1: 210464364-210464883 | 1: 211678056-211678476 |
| 1: 211734285-211734705 | 1: 211734536-211734933 | 1: 212514297-212515104 |
| 1: 212916505-212916925 | 1: 213104237-213104627 | 1: 213979863-213980283 |
| 1: 214224569-214224989 | 1: 214414829-214415247 | 1: 217574311-217574644 |
| 1: 219958526-219958946 | 1: 220689668-220690088 | 1: 220734720-220735145 |
| 1: 220887605-220888025 | 1: 220891987-220892407 | 1: 222916811-222917231 |
| 1: 223174858-223175274 | 1: 225477864-225478284 | 1: 227431803-227432223 |
| 1: 227887567-227887987 | 1: 228213907-228214363 | 1: 228321858-228322278 |
| 1: 228417036-228417547 | 1: 229230114-229230534 | 1: 229410562-229410982 |
| 1: 229433691-229434111 | 1: 229514841-229515261 | 1: 230424218-230424638 |
| 1: 231238840-231239260 | 1: 231577204-231577695 | 1: 233025008-233025424 |
| 1: 233297018-233297328 | 1: 234643337-234643744 | 1: 234899628-234900048 |
| 1: 235083142-235083562 | 1: 235295726-235296146 | 1: 235486329-235486749 |
| 1: 235630416-235630836 | 1: 236747475-236747895 | 1: 237915394-237915870 |
| 1: 238485876-238486296 | 1: 240150295-240150599 | 1: 240888593-240889013 |
| 1: 241134643-241135159 | 1: 241703098-241703518 | 1: 243107026-243107446 |
| 1: 243107304-243107724 | 1: 246281351-246281723 | 1: 246775647-246776067 |
| 1: 246929982-246930402 | 1: 247169032-247169452 | 1: 247451639-247452059 |
| 1: 248122480-248122888 | 10: 235359-235795 | 10: 652125-652545 |
| 10: 825049-825444 | 10: 847886-848293 | 10: 944427-944847 |
| 10: 1107681-1108096 | 10: 3811267-3811731 | 10: 3852729-3853078 |
| 10: 4058178-4058577 | 10: 4122157-4122577 | 10: 4650825-4651181 |
| 10: 5442364-5442731 | 10: 5840296-5840716 | 10: 6001243-6001594 |
| 10: 6013409-6013829 | 10: 6117676-6118105 | 10: 6128694-6129114 |
| 10: 6145540-6146030 | 10: 6382135-6382555 | 10: 6417295-6417715 |
| 10: 6464977-6465362 | 10: 6478868-6479277 | 10: 6489681-6490086 |
| 10: 6530220-6530640 | 10: 6583308-6583778 | 10: 6748618-6749060 |
| 10: 6751390-6751750 | 10: 6831624-6832043 | 10: 6831850-6832310 |
| 10: 6832402-6832782 | 10: 7288303-7288660 | 10: 7475501-7475970 |
| 10: 8034064-8034484 | 10: 8037069-8037489 | 10: 8260650-8261070 |
| 10: 9825779-9826156 | 10: 9878037-9878537 | 10: 10936746-10937159 |
| 10: 11259995-11260415 | 10: 11288282-11288702 | 10: 11339586-11340006 |
| 10: 11343310-11343725 | 10: 11378091-11378511 | 10: 11385940-11386314 |
| 10: 11424269-11424689 | 10: 11441068-11441488 | 10: 11662610-11663030 |
| 10: 11695196-11695616 | 10: 12713168-12713630 | 10: 12729890-12730300 |
| 10: 13001019-13001439 | 10: 13234732-13235152 | 10: 14497519-14497905 |
| 10: 14542993-14543413 | 10: 14572356-14572776 | 10: 14775048-14775468 |
| 10: 15254628-15255048 | 10: 15684712-15685132 | 10: 17233449-17233851 |
| 10: 21166863-21167261 | 10: 21993187-21993639 | 10: 22345607-22346328 |
| 10: 26503088-26503508 | 10: 26534359-26534764 | 10: 26579455-26579862 |
| 10: 26586154-26586574 | 10: 26588656-26589100 | 10: 26640464-26640884 |
| 10: 26643120-26643540 | 10: 26646090-26646510 | 10: 26647648-26648010 |
| 10: 27505861-27506281 | 10: 27741480-27742073 | 10: 29217267-29217785 |
| 10: 29240992-29241424 | 10: 29849321-29849741 | 10: 31960958-31961378 |
| 10: 33661940-33662360 | 10: 34254998-34255418 | 10: 34728739-34729105 |
| 10: 35639716-35640136 | 10: 38605121-38605541 | 10: 38730224-38730644 |
| 10: 43605815-43606318 | 10: 43789929-43790318 | 10: 43825471-43825891 |
| 10: 43853805-43854225 | 10: 43911542-43911916 | 10: 43938812-43939253 |
| 10: 44002448-44002905 | 10: 44005130-44005543 | 10: 44005648-44006068 |
| 10: 44171368-44171776 | 10: 44206419-44206839 | 10: 46063412-46063832 |
| 10: 46552623-46552992 | 10: 46556908-46557338 | 10: 47152602-47153022 |
| 10: 47325242-47325662 | 10: 47349251-47349671 | 10: 48476364-48476784 |
| 10: 48973218-48973638 | 10: 49037078-49037532 | 10: 49141935-49142318 |
| 10: 49162281-49162701 | 10: 49281807-49282227 | 10: 49322635-49323055 |
| 10: 49396395-49396815 | 10: 49610892-49611398 | 10: 49611238-49611658 |
| 10: 49611505-49612460 | 10: 49761451-49761871 | 10: 50126437-50126888 |
| 10: 52346328-52346717 | 10: 52389176-52389596 | 10: 52945714-52946134 |
| 10: 59234637-59234948 | 10: 59944460-59944880 | 10: 60186586-60187052 |
| 10: 62082623-62083043 | 10: 63130766-63131186 | 10: 68529814-68530245 |
| 10: 69595789-69596192 | 10: 70600347-70600767 | 10: 70710404-70710824 |
| 10: 71067047-71067537 | 10: 71292467-71292887 | 10: 71609598-71610018 |
| 10: 71706370-71706749 | 10: 71747803-71748225 | 10: 71946046-71946466 |
| 10: 72088555-72089157 | 10: 72216837-72217292 | 10: 72320775-72321323 |
| 10: 72449606-72450026 | 10: 72691370-72691790 | 10: 73889110-73889530 |
| 10: 74786358-74786851 | 10: 75395249-75395769 | 10: 75746253-75746673 |
| 10: 77386811-77387231 | 10: 77877818-77878243 | 10: 77878156-77878576 |
| 10: 78035203-78035545 | 10: 78248459-78249012 | 10: 78406970-78407430 |
| 10: 78974477-78974897 | 10: 79285916-79286336 | 10: 79322963-79323383 |
| 10: 80356343-80356763 | 10: 84201676-84202065 | 10: 85613439-85613853 |
| 10: 86138749-86139169 | 10: 86154640-86155052 | 10: 86536334-86536754 |
| 10: 86830336-86830756 | 10: 87008433-87008853 | 10: 89558837-89559257 |
| 10: 89837781-89838226 | 10: 93075143-93075646 | 10: 93516740-93517160 |
| 10: 93745034-93745454 | 10: 95866037-95866345 | 10: 96208619-96209039 |
| 10: 96292603-96293018 | 10: 96673302-96673861 | 10: 97714906-97715428 |
| 10: 99484910-99485315 | 10: 99536117-99536537 | 10: 99894758-99895161 |
| 10: 100114949- | 10: 100562704- | 10: 100659236- |
| 100115369 | 100563124 | 100659656 |
| 10: 101127066- | 10: 101132621- | 10: 101136638- |
| 101127528 | 101133050 | 101137058 |
| 10: 101236583- | 10: 101260933- | 10: 101310587- |
| 101237118 | 101261353 | 101311007 |
| 10: 101616879- | 10: 102242032- | 10: 103584177- |
| 101617299 | 102242452 | 103584733 |
| 10: 103757778- | 10: 103772073- | 10: 106629160- |
| 103758198 | 103772493 | 106629580 |
| 10: 106969516- | 10: 110330607- | 10: 110330679- |
| 106969936 | 110331027 | 110331099 |
| 10: 110790763- | 10: 110834696- | 10: 111078125- |
| 110791157 | 110835116 | 111078609 |
| 10: 112601375- | 10: 114538396- | 10: 119319463- |
| 112601725 | 114538816 | 119319883 |
| 10: 119607974- | 10: 123857391- | 10: 123857543- |
| 119608394 | 123857811 | 123857963 |
| 10: 124036061- | 10: 124354729- | 10: 124719289- |
| 124036481 | 124355262 | 124719772 |
| 10: 124765961- | 10: 125084718- | 10: 125756439- |
| 124766381 | 125085192 | 125756859 |
| 10: 125859494- | 10: 126175934- | 10: 126504613- |
| 125859909 | 126176418 | 126505132 |
| 10: 127279616- | 10: 127908814- | 10: 128069814- |
| 127280036 | 127909188 | 128070234 |
| 10: 129544127- | 10: 129950038- | 10: 129958253- |
| 129544547 | 129950458 | 129958673 |
| 10: 129960206- | 10: 131072049- | 10: 131142411- |
| 129960626 | 131072469 | 131142831 |
| 10: 131405105- | 10: 131496649- | 10: 131675512- |
| 131405525 | 131497075 | 131675957 |
| 10: 131675869- | 10: 131811321- | 10: 131811929- |
| 131676328 | 131811740 | 131812319 |
| 10: 131844592- | 10: 131993249- | 10: 132190832- |
| 131845012 | 131993669 | 132191253 |
| 10: 132206002- | 10: 132279720- | 10: 132634079- |
| 132206422 | 132280140 | 132634672 |
| 10: 132642651- | 10: 132820170- | 10: 132837187- |
| 132643099 | 132820590 | 132837607 |
| 10: 132869161- | 10: 132872495- | 10: 132884992- |
| 132869581 | 132872915 | 132885412 |
| 10: 132915325- | 10: 132916248- | 10: 132919118- |
| 132915795 | 132916668 | 132919538 |
| 10: 132919555- | 10: 132919919- | 10: 132922557- |
| 132919975 | 132920445 | 132922977 |
| 10: 132926019- | 10: 132960693- | 10: 132980661- |
| 132926439 | 132961113 | 132981081 |
| 10: 132997871- | 10: 133012769- | 10: 133018396- |
| 132998291 | 133013189 | 133018816 |
| 10: 133019574- | 10: 133021156- | 10: 133021795- |
| 133020029 | 133021576 | 133022215 |
| 10: 133026825- | 10: 133026986- | 10: 133062143- |
| 133027245 | 133027406 | 133062563 |
| 10: 133063010- | 10: 133064421- | 10: 133098358- |
| 133063568 | 133064841 | 133099058 |
| 10: 133122190- | 10: 133124377- | 10: 133126864- |
| 133122610 | 133124797 | 133127284 |
| 10: 133129261- | 10: 133142026- | 10: 133155068- |
| 133129679 | 133142446 | 133155488 |
| 10: 133179466- | 10: 133182612- | 10: 133183211- |
| 133179886 | 133183032 | 133183631 |
| 10: 133189060- | 10: 133201560- | 10: 133201830- |
| 133189480 | 133201980 | 133202250 |
| 10: 133202792- | 10: 133205024- | 10: 133215710- |
| 133203278 | 133205777 | 133216188 |
| 10: 133229170- | 10: 133241349- | 10: 133276894- |
| 133229590 | 133241819 | 133277314 |
| 10: 133309335- | 10: 133309633- | 10: 133379773- |
| 133309755 | 133310053 | 133380229 |
| 11: 304149-304570 | 11: 504009-504443 | 11: 569018-569480 |
| 11: 709205-709623 | 11: 725474-725894 | 11: 726541-726961 |
| 11: 737699-738119 | 11: 862932-863352 | 11: 1194888-1195308 |
| 11: 1252452-1252872 | 11: 1303117-1303688 | 11: 1434037-1434457 |
| 11: 1642512-1642932 | 11: 1648472-1648892 | 11: 1652188-1652608 |
| 11: 1652453-1652873 | 11: 1694107-1694527 | 11: 1694502-1694922 |
| 11: 1838497-1838917 | 11: 1854388-1854808 | 11: 1868532-1868952 |
| 11: 1869923-1870372 | 11: 1886575-1886995 | 11: 1919296-1919986 |
| 11: 1933722-1934142 | 11: 1937571-1937991 | 11: 2197705-2198125 |
| 11: 2202505-2202925 | 11: 2414273-2414693 | 11: 2414820-2415240 |
| 11: 2415172-2415592 | 11: 2480770-2481190 | 11: 2791058-2791510 |
| 11: 2849984-2850405 | 11: 2869537-2869957 | 11: 3165947-3166367 |
| 11: 3614623-3615043 | 11: 4212077-4212497 | 11: 4445133-4445452 |
| 11: 4605055-4605474 | 11: 5324535-5325026 | 11: 5507415-5507818 |
| 11: 5690760-5691180 | 11: 5820554-5820915 | 11: 5858781-5859201 |
| 11: 5936833-5937253 | 11: 5937402-5937822 | 11: 5937709-5938203 |
| 11: 6240452-6240872 | 11: 6769572-6769890 | 11: 6783489-6783909 |
| 11: 7076980-7077400 | 11: 7512394-7512814 | 11: 7575968-7576453 |
| 11: 7673761-7674369 | 11: 7851775-7852163 | 11: 9060321-9060733 |
| 11: 9660561-9661020 | 11: 9950524-9950890 | 11: 11352890-11353285 |
| 11: 12680287-12680707 | 11: 14905135-14905943 | 11: 16610888-16611308 |
| 11: 17386739-17387159 | 11: 17547134-17547616 | 11: 17631967-17632387 |
| 11: 17735851-17736858 | 11: 17738830-17739250 | 11: 18011599-18012062 |
| 11: 18106428-18106846 | 11: 18209168-18209588 | 11: 18264031-18264369 |
| 11: 19714335-19714755 | 11: 20159517-20160006 | 11: 22270110-22270524 |
| 11: 22341341-22341787 | 11: 31797681-31798101 | 11: 31807480-31807900 |
| 11: 31819695-31820116 | 11: 31820071-31820694 | 11: 31823411-31823979 |
| 11: 31824817-31825310 | 11: 32430484-32431187 | 11: 32437180-32437637 |
| 11: 32438258-32438690 | 11: 33014968-33015454 | 11: 33256489-33256909 |
| 11: 33808642-33809062 | 11: 34251050-34251470 | 11: 34513324-34513744 |
| 11: 36272136-36272522 | 11: 43522199-43522619 | 11: 43649461-43649881 |
| 11: 43811013-43811359 | 11: 44311312-44311814 | 11: 44568838-44569258 |
| 11: 45062957-45063410 | 11: 46345113-46345661 | 11: 46345988-46346447 |
| 11: 46690505-46690885 | 11: 46918697-46919117 | 11: 48048250-48048670 |
| 11: 48081304-48081724 | 11: 49433432-49433827 | 11: 49850361-49850781 |
| 11: 50170072-50170492 | 11: 50278695-50279115 | 11: 55276455-55276873 |
| 11: 56152235-56152655 | 11: 56277303-56277834 | 11: 57438625-57439036 |
| 11: 57467712-57468132 | 11: 57498619-57499033 | 11: 57639112-57639566 |
| 11: 59197964-59198406 | 11: 59200220-59200663 | 11: 59712873-59713293 |
| 11: 60040203-60040727 | 11: 60946712-60947086 | 11: 60950991-60951608 |
| 11: 61638044-61638464 | 11: 62169175-62169595 | 11: 62677888-62678348 |
| 11: 62687580-62688528 | 11: 62877189-62877609 | 11: 64229015-64229408 |
| 11: 64539253-64539673 | 11: 64590535-64590955 | 11: 64740163-64740583 |
| 11: 65104628-65104990 | 11: 65107740-65108160 | 11: 65116329-65116749 |
| 11: 65213353-65213773 | 11: 65576972-65577396 | 11: 65642156-65642657 |
| 11: 65786139-65786559 | 11: 65909243-65909663 | 11: 65956848-65957259 |
| 11: 66113839-66114344 | 11: 67082080-67082500 | 11: 67613880-67614322 |
| 11: 67665310-67665730 | 11: 68002915-68003546 | 11: 68021261-68021923 |
| 11: 68033558-68034013 | 11: 68256667-68257087 | 11: 68282120-68282576 |
| 11: 68298025-68298507 | 11: 68406494-68406892 | 11: 68416177-68416597 |
| 11: 68825628-68826048 | 11: 68854491-68854958 | 11: 69393360-69393780 |
| 11: 69533532-69533952 | 11: 69605780-69606200 | 11: 69653942-69654362 |
| 11: 69981205-69981566 | 11: 69984308-69984728 | 11: 70024488-70024908 |
| 11: 70141293-70141713 | 11: 70466369-70466748 | 11: 70661843-70662263 |
| 11: 70662127-70662547 | 11: 70671003-70671423 | 11: 70824599-70825019 |
| 11: 71628985-71629405 | 11: 71638931-71639336 | 11: 72590432-72590852 |
| 11: 73983601-73984021 | 11: 74071350-74071755 | 11: 74466877-74467297 |
| 11: 75404172-75404592 | 11: 75429564-75429984 | 11: 76148634-76149020 |
| 11: 76229700-76230076 | 11: 76234592-76235025 | 11: 76630285-76630705 |
| 11: 76752801-76753239 | 11: 77147524-77147944 | 11: 77192293-77192713 |
| 11: 77624936-77625356 | 11: 78420683-78421158 | 11: 82843503-82843863 |
| 11: 86053094-86053488 | 11: 87128137-87128529 | 11: 87972278-87972605 |
| 11: 88098059-88098400 | 11: 88292772-88293101 | 11: 88341948-88342319 |
| 11: 88385040-88385411 | 11: 90282575-90282995 | 11: 92882537-92882957 |
| 11: 92915659-92916079 | 11: 92969245-92969665 | 11: 92969494-92969914 |
| 11: 96215460-96215896 | 11: 96383965-96384363 | 11: 104036631- |
| | | 104037359 |
| 11: 104037267- | 11: 104909758- | 11: 105000164- |
| 104037979 | 104910167 | 105000584 |
| 11: 109940840- | 11: 110049939- | 11: 111514456- |
| 109941260 | 110050359 | 111514876 |
| 11: 111514652- | 11: 113231488- | 11: 116276985- |
| 111515129 | 113231908 | 116277395 |
| 11: 116500914- | 11: 117504701- | 11: 117522522- |
| 116501355 | 117505121 | 117522942 |
| 11: 117691378- | 11: 117947255- | 11: 118316486- |
| 117691798 | 117947780 | 118317037 |
| 11: 118532788- | 11: 118876710- | 11: 119149645- |
| 118533183 | 118877130 | 119150065 |
| 11: 120431944- | 11: 121038662- | 11: 121132205- |
| 120432364 | 121039013 | 121132625 |
| 11: 121468024- | 11: 121482534- | 11: 122092830- |
| 121468444 | 121482954 | 122093223 |
| 11: 122203256- | 11: 122248867- | 11: 122293559- |
| 122203676 | 122249245 | 122293979 |
| 11: 122360732- | 11: 122422690- | 11: 122448703- |
| 122361152 | 122423076 | 122449077 |
| 11: 122548380- | 11: 122579242- | 11: 122773186- |
| 122548800 | 122579641 | 122773570 |
| 11: 122902942- | 11: 122976304- | 11: 123314799- |
| 122903299 | 122976724 | 123315219 |
| 11: 123362972- | 11: 123577185- | 11: 123577520- |
| 123363437 | 123577605 | 123577940 |
| 11: 123579221- | 11: 123594452- | 11: 123754478- |
| 123579641 | 123594872 | 123754826 |
| 11: 123795989- | 11: 124124200- | 11: 124185386- |
| 123796417 | 124124620 | 124185784 |
| 11: 124311322- | 11: 124441246- | 11: 124758727- |
| 124311727 | 124441665 | 124759844 |
| 11: 124761428- | 11: 124876847- | 11: 125489462- |
| 124761848 | 124877649 | 125489882 |
| 11: 127471278- | 11: 128157045- | 11: 128400826- |
| 127471670 | 128157427 | 128401231 |
| 11: 128549219- | 11: 128867155- | 11: 128904421- |
| 128549639 | 128867575 | 128904841 |
| 11: 128908436- | 11: 128926360- | 11: 129852353- |
| 128908881 | 128926780 | 129852783 |
| 11: 130449946- | 11: 131868459- | 11: 132292231- |
| 130450453 | 131868857 | 132292610 |
| 11: 133078013- | 11: 133865031- | 11: 134008372- |
| 133078433 | 133865451 | 134008764 |
| 11: 134142721- | 11: 134207734- | 11: 134259012- |
| 134143096 | 134208154 | 134259427 |
| 11: 134599343- | 12: 101977-102398 | 12: 137907-138327 |
| 134599829 | | |
| 12: 138245-138665 | 12: 547348-547768 | 12: 643325-643801 |
| 12: 1536702-1537081 | 12: 1867354-1867667 | 12: 1885637-1886057 |
| 12: 2159903-2160293 | 12: 2624770-2625190 | 12: 2640655-2641075 |
| 12: 2739134-2739534 | 12: 2752989-2753409 | 12: 3803549-3803957 |
| 12: 4006570-4006958 | 12: 4143889-4144251 | 12: 4302887-4303285 |
| 12: 4445226-4445646 | 12: 4724745-4725108 | 12: 4814239-4814657 |
| 12: 4912396-4912816 | 12: 4915270-4915708 | 12: 4977658-4978029 |
| 12: 5430167-5430640 | 12: 6063432-6063792 | 12: 6385130-6385621 |
| 12: 6773484-6773904 | 12: 6824050-6824580 | 12: 6967539-6967912 |
| 12: 6967734-6968156 | 12: 7872576-7872996 | 12: 8222089-8222509 |
| 12: 8224647-8225067 | 12: 8245902-8246322 | 12: 8319034-8319454 |
| 12: 8373166-8373530 | 12: 8460541-8460961 | 12: 8512276-8512688 |
| 12: 9325119-9325516 | 12: 10169350-10169743 | 12: 10506986-10507406 |
| 12: 10615318-10615738 | 12: 11765826-11766246 | 12: 12313931-12314288 |
| 12: 13567305-13567680 | 12: 16359782-16360288 | 12: 16604928-16605348 |
| 12: 18882410-18882830 | 12: 21574055-21574447 | 12: 24583919-24584339 |
| 12: 24704324-24704744 | 12: 24714999-24715397 | 12: 25803063-25803465 |
| 12: 27067491-27067911 | 12: 29150017-29150437 | 12: 29792731-29793150 |
| 12: 31985734-31986154 | 12: 32252479-32252864 | 12: 32258897-32259317 |
| 12: 32985970-32986390 | 12: 33588818-33589220 | 12: 34205639-34206059 |
| 12: 34358725-34359184 | 12: 37566351-37566771 | 12: 38150878-38151298 |
| 12: 38823614-38823987 | 12: 40239098-40239518 | 12: 40567462-40567923 |
| 12: 41412356-41412736 | 12: 42287152-42287572 | 12: 42697314-42697734 |
| 12: 45065257-45065677 | 12: 47097517-47097927 | 12: 47808048-47808468 |
| 12: 47817048-47817468 | 12: 47882808-47883272 | 12: 48012256-48012601 |
| 12: 48184170-48185069 | 12: 48198608-48199152 | 12: 49332728-49333481 |
| 12: 49335906-49336326 | 12: 49341791-49342211 | 12: 49539431-49539851 |
| 12: 49549288-49549708 | 12: 49757883-49758303 | 12: 49955385-49955805 |
| 12: 51721244-51721664 | 12: 51721695-51722115 | 12: 52007589-52008011 |
| 12: 52600969-52601668 | 12: 52770910-52771330 | 12: 52898538-52899017 |
| 12: 52951374-52951764 | 12: 53217217-53217690 | 12: 53677211-53677631 |
| 12: 53733718-53734138 | 12: 53748014-53748443 | 12: 53956178-53956598 |
| 12: 53968758-53969178 | 12: 54187922-54188342 | 12: 54657782-54658202 |
| 12: 55830745-55831165 | 12: 56043289-56043643 | 12: 56099755-56100175 |
| 12: 56338670-56339090 | 12: 56643204-56643624 | 12: 57030857-57031277 |
| 12: 57236164-57236584 | 12: 62800020-62800420 | 12: 63149705-63150125 |
| 12: 64390127-64390547 | 12: 65100381-65100801 | 12: 65192897-65193317 |
| 12: 65881612-65882040 | 12: 65882052-65882472 | 12: 66065084-66065445 |
| 12: 68253307-68253727 | 12: 75480615-75481035 | 12: 75490282-75490702 |
| 12: 75755319-75755665 | 12: 79614649-79615043 | 12: 84914178-84914591 |
| 12: 87784252-87784672 | 12: 93154870-93155290 | 12: 95384007-95384407 |
| 12: 96448406-96448735 | 12: 100716626- | 12: 101618129- |
| | 100717046 | 101618517 |
| 12: 102062546- | 12: 102959347- | 12: 104695571- |
| 102062966 | 102959881 | 104695991 |
| 12: 104723112- | 12: 104966726- | 12: 105083561- |
| 104723532 | 104967061 | 105084067 |
| 12: 106580485- | 12: 106773414- | 12: 107013960- |
| 106580926 | 106773834 | 107014307 |
| 12: 107682168- | 12: 108046226- | 12: 108288461- |
| 107682588 | 108046738 | 108288816 |
| 12: 108538799- | 12: 108638111- | 12: 109291629- |
| 108539195 | 108638531 | 109292243 |
| 12: 109311581- | 12: 109388575- | 12: 109463273- |
| 109311989 | 109388995 | 109463693 |
| 12: 109606565- | 12: 109718277- | 12: 109734107- |
| 109606985 | 109718853 | 109734527 |
| 12: 109734279- | 12: 109736553- | 12: 109794319- |
| 109734699 | 109736980 | 109794739 |
| 12: 109927480- | 12: 111032754- | 12: 111298710- |
| 109927900 | 111033174 | 111299130 |
| 12: 112239727- | 12: 112252213- | 12: 112294843- |
| 112240147 | 112252633 | 112295263 |
| 12: 112406527- | 12: 114404019- | 12: 114505088- |
| 112407010 | 114404439 | 114505508 |
| 12: 114686775- | 12: 114687380- | 12: 116018001- |
| 114687538 | 114688001 | 116018413 |
| 12: 116140502- | 12: 116492605- | 12: 116857962- |
| 116140913 | 116493025 | 116858382 |
| 12: 117032727- | 12: 117045037- | 12: 117045259- |
| 117033147 | 117045457 | 117045679 |
| 12: 117875954- | 12: 120142425- | 12: 121237866- |
| 117876420 | 120142845 | 121238318 |
| 12: 121240486- | 12: 121279665- | 12: 121336596- |
| 121240906 | 121280085 | 121337016 |
| 12: 121400782- | 12: 121535983- | 12: 121550804- |
| 121401372 | 121536403 | 121551195 |
| 12: 121578795- | 12: 121580989- | 12: 121754931- |
| 121579331 | 121581409 | 121755351 |
| 12: 122191232- | 12: 122508252- | 12: 123268091- |
| 122191652 | 122508672 | 123268557 |
| 12: 123269345- | 12: 123398170- | 12: 123913112- |
| 123269765 | 123398590 | 123913530 |
| 12: 124053937- | 12: 124347974- | 12: 124543081- |
| 124054357 | 124348394 | 124543501 |
| 12: 124604904- | 12: 124646328- | 12: 124864400- |
| 124605324 | 124646748 | 124864820 |
| 12: 127294044- | 12: 127456077- | 12: 128233922- |
| 127294497 | 127456497 | 128234342 |
| 12: 128414903- | 12: 128544037- | 12: 128572295- |
| 128415323 | 128544457 | 128572712 |
| 12: 128645178- | 12: 128705123- | 12: 128708476- |
| 128645650 | 128705543 | 128708871 |
| 12: 128725936- | 12: 128932475- | 12: 129036052- |
| 128726319 | 128932895 | 129036472 |
| 12: 130336689- | 12: 130401914- | 12: 130468849- |
| 130337109 | 130402334 | 130469269 |
| 12: 130704636- | 12: 130711672- | 12: 130999737- |
| 130705056 | 130712092 | 131000157 |
| 12: 131066647- | 12: 131070474- | 12: 131084487- |
| 131067067 | 131070894 | 131084907 |
| 12: 131087978- | 12: 131105606- | 12: 131108508- |
| 131088398 | 131106026 | 131108928 |
| 12: 131121857- | 12: 131202881- | 12: 131235902- |
| 131122277 | 131203301 | 131236322 |
| 12: 131236991- | 12: 131280989- | 12: 131329444- |
| 131237474 | 131281409 | 131329864 |
| 12: 131345060- | 12: 131349651- | 12: 131371000- |
| 131345480 | 131350071 | 131371427 |
| 12: 131425492- | 12: 131456778- | 12: 131529627- |
| 131425852 | 131457198 | 131530047 |
| 12: 131560290- | 12: 131569887- | 12: 131660443- |
| 131560683 | 131570290 | 131660863 |
| 12: 131687619- | 12: 131844546- | 12: 131848614- |
| 131688039 | 131844966 | 131849034 |
| 12: 132179177- | 12: 132203542- | 12: 132205744- |
| 132179532 | 132203962 | 132206164 |
| 12: 132257645- | 12: 132269119- | 12: 132292214- |
| 132258065 | 132269539 | 132292634 |
| 12: 132331681- | 12: 132391196- | 12: 132391634- |
| 132332098 | 132391616 | 132392069 |
| 12: 132395969- | 12: 132601124- | 12: 132700596- |
| 132396389 | 132601553 | 132700902 |
| 13: 18665645-18666065 | 13: 18726676-18727090 | 13: 18837842-18838356 |
| 13: 18952520-18952939 | 13: 19305391-19305811 | 13: 19390506-19390926 |
| 13: 20142189-20142609 | 13: 20193730-20194150 | 13: 20301318-20301837 |
| 13: 20301575-20301995 | 13: 20702412-20702832 | 13: 20721321-20721741 |
| 13: 21526603-21527023 | 13: 21766781-21767201 | 13: 22119731-22120133 |
| 13: 22839983-22840403 | 13: 22880414-22880834 | 13: 22926626-22927046 |
| 13: 23715085-23715549 | 13: 24274375-24274795 | 13: 24692355-24692735 |
| 13: 24889118-24889538 | 13: 26012243-26012688 | 13: 26785203-26785623 |
| 13: 26914626-26915046 | 13: 27791336-27791756 | 13: 27917940-27918553 |
| 13: 27918658-27919078 | 13: 27928360-27928780 | 13: 27964733-27965153 |
| 13: 27970119-27970600 | 13: 28328226-28328646 | 13: 28778550-28778970 |
| 13: 30445547-30446108 | 13: 30739928-30740348 | 13: 30740244-30740664 |
| 13: 30865633-30866002 | 13: 30906014-30906434 | 13: 35475023-35475774 |
| 13: 41512183-41512603 | 13: 44573173-44573531 | 13: 46135671-46136091 |
| 13: 49220593-49221332 | 13: 49628179-49628599 | 13: 50133327-50133747 |
| 13: 50843543-50843963 | 13: 51380146-51380491 | 13: 52195243-52195893 |
| 13: 52377162-52377582 | 13: 52739327-52740158 | 13: 53200640-53201060 |
| 13: 54312965-54313385 | 13: 57633003-57633426 | 13: 57634131-57634551 |
| 13: 60572766-60573186 | 13: 67841059-67841426 | 13: 73137999-73138337 |
| 13: 73962330-73962720 | 13: 74052316-74052734 | 13: 75385375-75385786 |
| 13: 75993880-75994283 | 13: 76519684-76520104 | 13: 77050037-77050445 |
| 13: 77918342-77918762 | 13: 78595365-78595785 | 13: 78596928-78597397 |
| 13: 78606881-78607301 | 13: 78607588-78608008 | 13: 80655322-80655760 |
| 13: 83906465-83906885 | 13: 83907151-83907571 | 13: 83907389-83907809 |
| 13: 87810951-87811419 | 13: 94262957-94263316 | 13: 95044328-95044684 |
| 13: 95236691-95237212 | 13: 95523618-95524038 | 13: 99432569-99433271 |
| 13: 99895674-99896608 | 13: 99960836-99961261 | 13: 99984397-99984967 |
| 13: 99990026-99990446 | 13: 99992253-99992698 | 13: 99997365-99997954 |
| 13: 102916262- | 13: 108960886- | 13: 109140265- |
| 102916665 | 108961306 | 109140685 |
| 13: 110352655- | 13: 110379625- | 13: 110382235- |
| 110353075 | 110380045 | 110382544 |
| 13: 110387431- | 13: 110424695- | 13: 110438126- |
| 110387851 | 110425115 | 110438546 |
| 13: 110446211- | 13: 110618122- | 13: 110812262- |
| 110446631 | 110618542 | 110813021 |
| 13: 110869692- | 13: 111093209- | 13: 111177021- |
| 110870175 | 111093629 | 111177441 |
| 13: 111548073- | 13: 111556703- | 13: 111567667- |
| 111548493 | 111557123 | 111568087 |
| 13: 111604169- | 13: 111956731- | 13: 112037605- |
| 111604589 | 111957151 | 112038109 |
| 13: 112169144- | 13: 112202456- | 13: 112240994- |
| 112169564 | 112202876 | 112241414 |
| 13: 112338496- | 13: 112355280- | 13: 112441233- |
| 112338893 | 112355700 | 112441653 |
| 13: 112756978- | 13: 112759797- | 13: 112770735- |
| 112757398 | 112760158 | 112771160 |
| 13: 112941488- | 13: 113026416- | 13: 113110835- |
| 112941884 | 113026836 | 113111255 |
| 13: 113400402- | 13: 113407627- | 13: 113455484- |
| 113400822 | 113408047 | 113455907 |
| 13: 113908349- | 13: 113913563- | 13: 114017212- |
| 113908769 | 113913983 | 114017620 |
| 13: 114130955- | 13: 114147422- | 13: 114147683- |
| 114131473 | 114147842 | 114148103 |
| 14: 20430394-20430814 | 14: 21092569-21092989 | 14: 21526736-21527156 |
| 14: 21868002-21868379 | 14: 22111603-22112023 | 14: 22848075-22848495 |
| 14: 23237372-23237792 | 14: 23260810-23261230 | 14: 23307610-23308017 |
| 14: 23384491-23384911 | 14: 23419237-23419657 | 14: 23420023-23420443 |
| 14: 23432499-23432919 | 14: 23575863-23576341 | 14: 23954831-23955247 |
| 14: 24095010-24095430 | 14: 24311174-24311594 | 14: 24332596-24333097 |
| 14: 24368894-24369314 | 14: 24402914-24403311 | 14: 24982289-24982702 |
| 14: 27633933-27634353 | 14: 28767851-28768271 | 14: 28768175-28768764 |
| 14: 35369554-35369966 | 14: 36508771-36509335 | 14: 36583242-36583662 |
| 14: 38209600-38210094 | 14: 39215806-39216226 | 14: 41605934-41606354 |
| 14: 43300114-43300534 | 14: 46384700-46385083 | 14: 46651323-46651743 |
| 14: 47200706-47201258 | 14: 49961681-49962101 | 14: 50677517-50677937 |
| 14: 50831803-50832223 | 14: 50833376-50833754 | 14: 53950269-53950676 |
| 14: 54211682-54212052 | 14: 54776283-54776703 | 14: 55024140-55024557 |
| 14: 56797123-56797543 | 14: 56798965-56799489 | 14: 57982081-57982473 |
| 14: 58154978-58155398 | 14: 60244828-60245195 | 14: 60643595-60644226 |
| 14: 61320570-61320980 | 14: 61471705-61472125 | 14: 61559815-61560235 |
| 14: 61751224-61751644 | 14: 64541982-64542629 | 14: 64705206-64705912 |
| 14: 65266743-65267163 | 14: 67690072-67690492 | 14: 68377335-68377755 |
| 14: 68550228-68550557 | 14: 69400879-69401282 | 14: 69615443-69615863 |
| 14: 70919755-70920175 | 14: 71116806-71117175 | 14: 72215395-72215737 |
| 14: 72453626-72453949 | 14: 72463690-72464077 | 14: 72510068-72510488 |
| 14: 72511329-72511718 | 14: 72547099-72547533 | 14: 72547675-72548033 |
| 14: 72547859-72548279 | 14: 72549153-72549573 | 14: 72557818-72558238 |
| 14: 72611722-72612142 | 14: 72619493-72619901 | 14: 72619762-72620182 |
| 14: 72688812-72689254 | 14: 72709107-72709527 | 14: 72711517-72711937 |
| 14: 72731450-72731856 | 14: 72742735-72743106 | 14: 72802098-72802493 |
| 14: 73245431-73246113 | 14: 73998166-73998586 | 14: 74611038-74611778 |
| 14: 74879334-74879713 | 14: 74922258-74922678 | 14: 75084643-75085063 |
| 14: 75100872-75101301 | 14: 76131342-76131762 | 14: 76134148-76134568 |
| 14: 76862685-76863105 | 14: 76867422-76867842 | 14: 76968464-76968884 |
| 14: 77681040-77681460 | 14: 81427251-81427671 | 14: 85621986-85622406 |
| 14: 89175944-89176330 | 14: 89242037-89242389 | 14: 89303788-89304208 |
| 14: 89401571-89401991 | 14: 89641552-89641934 | 14: 89720892-89721312 |
| 14: 90254037-90254416 | 14: 91244518-91244972 | 14: 91268162-91268574 |
| 14: 91290530-91290884 | 14: 91323912-91324332 | 14: 91367111-91367622 |
| 14: 91652976-91653291 | 14: 91832694-91833045 | 14: 92946140-92946560 |
| 14: 93079948-93080387 | 14: 93135350-93135712 | 14: 93654531-93654951 |
| 14: 94773656-94774369 | 14: 95273694-95274114 | 14: 95507162-95507677 |
| 14: 95686183-95686603 | 14: 96242186-96242606 | 14: 96263953-96264373 |
| 14: 96931219-96931639 | 14: 97180615-97181035 | 14: 97299421-97299812 |
| 14: 97383408-97383828 | 14: 97408949-97409369 | 14: 97426912-97427313 |
| 14: 99118323-99118775 | 14: 99319675-99320057 | 14: 99348560-99348980 |
| 14: 99660087-99660507 | 14: 99899764-99900120 | 14: 100038262- |
| | | 100038680 |
| 14: 100187617- | 14: 100689011- | 14: 100735057- |
| 100188036 | 100689431 | 100735477 |
| 14: 100736212- | 14: 100865797- | 14: 100873484- |
| 100736629 | 100866217 | 100873904 |
| 14: 100883049- | 14: 100884207- | 14: 100884605- |
| 100883469 | 100884627 | 100885011 |
| 14: 100891091- | 14: 101023185- | 14: 101023580- |
| 100891511 | 101023605 | 101024000 |
| 14: 101025497- | 14: 101029161- | 14: 101052292- |
| 101025917 | 101029572 | 101052712 |
| 14: 101054092- | 14: 101054456- | 14: 101054906- |
| 101054512 | 101054834 | 101055326 |
| 14: 101059714- | 14: 101062042- | 14: 101064904- |
| 101060120 | 101062462 | 101065324 |
| 14: 101065618- | 14: 101066001- | 14: 101590791- |
| 101066038 | 101066421 | 101591211 |
| 14: 101705601- | 14: 102084756- | 14: 102088317- |
| 101706021 | 102085304 | 102088785 |
| 14: 102206953- | 14: 102923454- | 14: 103071603- |
| 102207373 | 102923874 | 103072023 |
| 14: 103102090- | 14: 103138529- | 14: 103274242- |
| 103102510 | 103139068 | 103274705 |
| 14: 103313990- | 14: 103698488- | 14: 103738747- |
| 103314410 | 103698908 | 103739167 |
| 14: 104164104- | 14: 104170562- | 14: 104179024- |
| 104164524 | 104170982 | 104179444 |
| 14: 104179953- | 14: 104223277- | 14: 104223975- |
| 104180365 | 104223697 | 104224395 |
| 14: 104637007- | 14: 104678217- | 14: 104681175- |
| 104637479 | 104678795 | 104681595 |
| 14: 104793340- | 14: 104906785- | 14: 105029319- |
| 104793760 | 104907205 | 105029676 |
| 14: 105192081- | 14: 105221817- | 14: 105249428- |
| 105192919 | 105222237 | 105249848 |
| 14: 105249608- | 14: 105294050- | 14: 105678074- |
| 105250028 | 105294470 | 105678494 |
| 14: 106286526- | 14: 106286795- | 14: 106436173- |
| 106286946 | 106287215 | 106436593 |
| 14: 106459794- | 14: 106459950- | 14: 106665940- |
| 106460214 | 106460370 | 106666360 |
| 15: 20440336-20440770 | 15: 20541735-20542114 | 15: 22775900-22776320 |
| 15: 25780334-25780754 | 15: 26866178-26866598 | 15: 27806211-27806631 |
| 15: 28848424-28848844 | 15: 31392778-31393448 | 15: 34495569-34496014 |
| 15: 34754231-34754651 | 15: 36361239-36361659 | 15: 37095087-37095563 |
| 15: 37097105-37097577 | 15: 37097801-37098329 | 15: 37490461-37491231 |
| 15: 37820759-37821169 | 15: 37823394-37823857 | 15: 38069010-38069449 |
| 15: 40510991-40511583 | 15: 40925482-40926050 | 15: 42226676-42227053 |
| 15: 42226860-42227280 | 15: 42679602-42680014 | 15: 43801581-43801960 |
| 15: 45039633-45039967 | 15: 45113895-45114315 | 15: 45116255-45116778 |
| 15: 45128720-45129458 | 15: 45454717-45455245 | 15: 45520780-45521146 |
| 15: 45704216-45704773 | 15: 51951726-51952146 | 15: 52250408-52250828 |
| 15: 52812812-52813169 | 15: 57528467-57528887 | 15: 57565258-57565678 |
| 15: 58064873-58065774 | 15: 58864670-58865090 | 15: 59144425-59144845 |
| 15: 60009957-60010377 | 15: 62119224-62119644 | 15: 63677735-63678150 |
| 15: 64159076-64159495 | 15: 64893675-64894226 | 15: 64905146-64905864 |
| 15: 65382034-65382454 | 15: 66671225-66671645 | 15: 67033534-67034220 |
| 15: 67819459-67820051 | 15: 67825720-67826281 | 15: 68691954-68692288 |
| 15: 68700533-68700953 | 15: 68795201-68795621 | 15: 70140397-70140817 |
| 15: 70447902-70448361 | 15: 73442899-73443319 | 15: 74065327-74065696 |
| 15: 74124774-74125174 | 15: 74135136-74135556 | 15: 74156962-74157382 |
| 15: 74618918-74619338 | 15: 74928872-74929272 | 15: 75195441-75195861 |
| 15: 75202993-75203413 | 15: 75791114-75791534 | 15: 78034777-78035197 |
| 15: 78429845-78430159 | 15: 78685560-78686036 | 15: 78811732-78812152 |
| 15: 78830808-78831405 | 15: 78937150-78937570 | 15: 79432784-79433204 |
| 15: 79902186-79902606 | 15: 80704292-80704712 | 15: 80749259-80749679 |
| 15: 80906468-80906888 | 15: 81133940-81134360 | 15: 81134133-81134553 |
| 15: 82692042-82692462 | 15: 82844663-82845114 | 15: 84606716-84607187 |
| 15: 84855423-84855813 | 15: 87755274-87755652 | 15: 88258146-88258569 |
| 15: 88603933-88604353 | 15: 88858998-88859418 | 15: 89116201-89116635 |
| 15: 89204600-89205013 | 15: 89378745-89379593 | 15: 89409498-89409918 |
| 15: 89647396-89647816 | 15: 89665777-89666197 | 15: 89747689-89748082 |
| 15: 89888569-89888989 | 15: 90232199-90232618 | 15: 90986809-90987190 |
| 15: 91293878-91294221 | 15: 91968647-91969062 | 15: 92053347-92053767 |
| 15: 92171744-92172164 | 15: 92384032-92384394 | 15: 92618064-92618434 |
| 15: 93430716-93431136 | 15: 93900574-93900994 | 15: 94855641-94856061 |
| 15: 94855988-94856408 | 15: 96366319-96366931 | 15: 96758118-96758538 |
| 15: 97522426-97522846 | 15: 97597860-97598367 | 15: 97873807-97874338 |
| 15: 98403620-98404040 | 15: 98687064-98687467 | 15: 98803814-98804236 |
| 15: 99105935-99106355 | 15: 99425099-99425629 | 15: 99476869-99477289 |
| 15: 100381156- | 15: 100869206- | 15: 100973747- |
| 100381576 | 100869626 | 100974167 |
| 15: 101273690- | 15: 101320741- | 15: 101376958- |
| 101274088 | 101321161 | 101377378 |
| 15: 101501867- | 15: 101752370- | 15: 101759527- |
| 101502287 | 101752790 | 101759947 |
| 15: 101763923- | 15: 101764647- | 15: 101767200- |
| 101764343 | 101765067 | 101767620 |
| 15: 101767392- | 16: 47218-47638 | 16: 76297-76717 |
| 101767812 | | |
| 16: 331673-332118 | 16: 379856-380229 | 16: 386733-387350 |
| 16: 598760-599159 | 16: 627446-627858 | 16: 681270-681690 |
| 16: 682649-683500 | 16: 684665-685085 | 16: 757601-758037 |
| 16: 780517-780937 | 16: 787296-787785 | 16: 795601-796017 |
| 16: 810647-811067 | 16: 847049-847469 | 16: 852907-853327 |
| 16: 1323271-1323691 | 16: 1350621-1351093 | 16: 1365875-1366295 |
| 16: 1428448-1428868 | 16: 1440980-1441478 | 16: 1525780-1526396 |
| 16: 1545330-1545750 | 16: 1680324-1680744 | 16: 1898762-1899181 |
| 16: 1923501-1923921 | 16: 1961912-1962370 | 16: 2043983-2044403 |
| 16: 2090434-2090854 | 16: 2211859-2212279 | 16: 2226618-2226998 |
| 16: 2264163-2264583 | 16: 2264525-2264945 | 16: 2756183-2756603 |
| 16: 2842463-2842883 | 16: 3102050-3102470 | 16: 3175323-3175743 |
| 16: 3191099-3191519 | 16: 3678216-3678570 | 16: 3881418-3881846 |
| 16: 3912351-3912771 | 16: 3988966-3989386 | 16: 4032727-4033168 |
| 16: 4330483-4330903 | 16: 4912246-4912666 | 16: 5084559-5084979 |
| 16: 5117165-5117585 | 16: 5206909-5207329 | 16: 6483113-6483533 |
| 16: 8581912-8582332 | 16: 8901889-8902355 | 16: 8946721-8947141 |
| 16: 9090122-9090572 | 16: 9763119-9763605 | 16: 10111399-10111878 |
| 16: 10132447-10132898 | 16: 10172856-10173254 | 16: 10252179-10252582 |
| 16: 10627527-10627940 | 16: 10818205-10818625 | 16: 10818581-10819001 |
| 16: 10871091-10871511 | 16: 10877145-10877565 | 16: 11233002-11233614 |
| 16: 11328418-11328838 | 16: 11526959-11527379 | 16: 11759146-11759566 |
| 16: 12261006-12261622 | 16: 12843077-12843497 | 16: 15532759-15533179 |
| 16: 16022117-16022537 | 16: 16074309-16074768 | 16: 16572740-16573160 |
| 16: 17343015-17343435 | 16: 17557334-17557754 | 16: 17932747-17933198 |
| 16: 19118930-19119350 | 16: 19245572-19245992 | 16: 19479476-19479881 |
| 16: 19804914-19805334 | 16: 20075854-20076287 | 16: 20690897-20691317 |
| 16: 21148587-21148918 | 16: 21515272-21515692 | 16: 23336053-23336473 |
| 16: 23363123-23363483 | 16: 23642152-23642566 | 16: 23704719-23705139 |
| 16: 23790485-23790905 | 16: 23908274-23908591 | 16: 24016021-24016441 |
| 16: 24139719-24140126 | 16: 24351885-24352316 | 16: 24670865-24671285 |
| 16: 27214385-27214877 | 16: 27214730-27215150 | 16: 27511766-27512182 |
| 16: 28844422-28844842 | 16: 28882292-28882712 | 16: 28921008-28921404 |
| 16: 29074886-29075383 | 16: 29141871-29142538 | 16: 29160351-29160771 |
| 16: 29161183-29161603 | 16: 29188763-29189183 | 16: 29256684-29257104 |
| 16: 29784891-29785540 | 16: 29809073-29809486 | 16: 29876981-29877401 |
| 16: 30011925-30012345 | 16: 30012054-30012474 | 16: 31127074-31127543 |
| 16: 31201541-31201961 | 16: 31214546-31215029 | 16: 31370923-31371343 |
| 16: 31994399-31994819 | 16: 31994764-31995184 | 16: 33767379-33767799 |
| 16: 33769020-33769440 | 16: 33927844-33928264 | 16: 47392043-47392519 |
| 16: 48059704-48060124 | 16: 48230024-48230433 | 16: 48536150-48536731 |
| 16: 49407018-49407438 | 16: 50840996-50841416 | 16: 50860071-50860472 |
| 16: 50995913-50996333 | 16: 51140007-51140425 | 16: 51154276-51154696 |
| 16: 54282842-54283370 | 16: 55737789-55738209 | 16: 56193571-56193991 |
| 16: 56589635-56590055 | 16: 56635055-56635719 | 16: 56635540-56635960 |
| 16: 57533738-57534158 | 16: 57726034-57726454 | 16: 57813522-57813942 |
| 16: 57840850-57841270 | 16: 57883827-57884247 | 16: 57906482-57906902 |
| 16: 59754800-59755318 | 16: 60359372-60359792 | 16: 64260952-64261372 |
| 16: 64439861-64440246 | 16: 66426276-66426696 | 16: 66549179-66549599 |
| 16: 66948537-66949192 | 16: 68232433-68232800 | 16: 68351649-68352032 |
| 16: 68792874-68793294 | 16: 69077870-69078222 | 16: 69107387-69107807 |
| 16: 69313460-69313880 | 16: 69771643-69772009 | 16: 69904902-69905306 |
| 16: 69909932-69910288 | 16: 71536796-71537216 | 16: 72024180-72024510 |
| 16: 73093377-73093870 | 16: 74627777-74628208 | 16: 77193565-77193985 |
| 16: 77963198-77963618 | 16: 78894859-78895292 | 16: 79047280-79047765 |
| 16: 79838551-79838971 | 16: 80027016-80027456 | 16: 80991682-80992102 |
| 16: 81053723-81054130 | 16: 81261270-81261665 | 16: 81350018-81350387 |
| 16: 81767642-81768062 | 16: 81889931-81890268 | 16: 81938222-81938552 |
| 16: 83804068-83804473 | 16: 83931044-83931464 | 16: 83968969-83969389 |
| 16: 84048268-84048655 | 16: 84579810-84580193 | 16: 84595251-84595664 |
| 16: 84703887-84704307 | 16: 84827970-84828347 | 16: 85082735-85083197 |
| 16: 85483464-85483884 | 16: 85483595-85484015 | 16: 85483809-85484229 |
| 16: 85636320-85636740 | 16: 85641421-85641841 | 16: 86016023-86016443 |
| 16: 86194830-86195250 | 16: 86216222-86216659 | 16: 86388189-86388704 |
| 16: 86394092-86394508 | 16: 86632152-86632572 | 16: 86752778-86753198 |
| 16: 86836111-86836531 | 16: 86870801-86871221 | 16: 86916855-86917275 |
| 16: 86924618-86925000 | 16: 86967786-86968206 | 16: 86986057-86986436 |
| 16: 87550597-87551017 | 16: 87550727-87551147 | 16: 87607314-87607734 |
| 16: 87864344-87864764 | 16: 87921371-87921791 | 16: 88122311-88122731 |
| 16: 88130076-88130496 | 16: 88154750-88155170 | 16: 88432063-88432483 |
| 16: 88436520-88436940 | 16: 88437013-88437433 | 16: 88452221-88452641 |
| 16: 88633947-88634367 | 16: 88650268-88650688 | 16: 88984162-88984582 |
| 16: 89085728-89086148 | 16: 89159432-89159852 | 16: 89161128-89161548 |
| 16: 89161294-89161714 | 16: 89200185-89200605 | 16: 89430136-89430597 |
| 16: 89562275-89562673 | 16: 89576372-89576792 | 16: 89608226-89608618 |
| 16: 89817955-89818375 | 16: 89818776-89819140 | 16: 89853450-89853870 |
| 16: 90004153-90004798 | 17: 749061-749599 | 17: 1006582-1007002 |
| 17: 1110788-1111208 | 17: 1111130-1111550 | 17: 1469212-1469632 |
| 17: 1486903-1487348 | 17: 1569421-1569841 | 17: 2036598-2037016 |
| 17: 2089432-2089918 | 17: 2595696-2596133 | 17: 2814462-2815372 |
| 17: 3309543-3309959 | 17: 3554336-3554756 | 17: 3665390-3665810 |
| 17: 4145531-4146000 | 17: 4785601-4786021 | 17: 4900970-4901390 |
| 17: 5299359-5299755 | 17: 5373046-5373466 | 17: 5514202-5514609 |
| 17: 6158629-6159049 | 17: 6425559-6425976 | 17: 6426716-6427136 |
| 17: 7174034-7174708 | 17: 7383795-7384299 | 17: 7437962-7438382 |
| 17: 7903708-7904128 | 17: 8163443-8163863 | 17: 9022758-9023178 |
| 17: 9232740-9233160 | 17: 9402721-9403141 | 17: 9905042-9905462 |
| 17: 9905190-9905610 | 17: 10177763-10178130 | 17: 10464516-10464917 |
| 17: 10613930-10614350 | 17: 10614136-10614556 | 17: 12036552-12036972 |
| 17: 13556994-13557347 | 17: 15782754-15783174 | 17: 16101057-16101477 |
| 17: 16415561-16415981 | 17: 17216773-17217192 | 17: 17663352-17663790 |
| 17: 17810616-17811036 | 17: 17812466-17813006 | 17: 18951290-18951710 |
| 17: 19306176-19306596 | 17: 19445763-19446183 | 17: 19446519-19446939 |
| 17: 19745229-19745649 | 17: 20291669-20292089 | 17: 20977389-20977809 |
| 17: 21082229-21082649 | 17: 21317029-21317487 | 17: 21359224-21359644 |
| 17: 21377379-21377998 | 17: 22090267-22090682 | 17: 22321970-22322390 |
| 17: 22692314-22692734 | 17: 27457114-27457570 | 17: 27591889-27592309 |
| 17: 27604183-27604523 | 17: 27653472-27653892 | 17: 27759700-27760103 |
| 17: 28227082-28227613 | 17: 28372313-28372733 | 17: 28528523-28528943 |
| 17: 28675745-28676165 | 17: 28684766-28685186 | 17: 28718261-28718681 |
| 17: 29639202-29639622 | 17: 30663732-30664152 | 17: 31390878-31391298 |
| 17: 31391986-31392406 | 17: 31803837-31804237 | 17: 32112415-32112835 |
| 17: 34637327-34637747 | 17: 35119037-35119476 | 17: 35444555-35444960 |
| 17: 35449395-35449815 | 17: 35449599-35450019 | 17: 35501192-35501612 |
| 17: 35510281-35510701 | 17: 35766565-35766985 | 17: 36722498-36722918 |
| 17: 36812754-36813174 | 17: 36940401-36940960 | 17: 38471375-38471795 |
| 17: 38561532-38561952 | 17: 38561831-38562251 | 17: 38562087-38562507 |
| 17: 38563198-38563800 | 17: 38563975-38564395 | 17: 38578270-38578879 |
| 17: 38900783-38901203 | 17: 39202812-39203232 | 17: 39204314-39204734 |
| 17: 40312270-40312690 | 17: 41379140-41379560 | 17: 41437960-41438380 |
| 17: 41762863-41763283 | 17: 41913775-41914195 | 17: 41967759-41968179 |
| 17: 41973472-41973892 | 17: 42107431-42108049 | 17: 42287434-42287854 |
| 17: 42312598-42313068 | 17: 42571336-42571756 | 17: 42571501-42571921 |
| 17: 42785073-42785612 | 17: 43006325-43006745 | 17: 43211952-43212423 |
| 17: 43953303-43953723 | 17: 45172758-45173178 | 17: 45243794-45244198 |
| 17: 45430468-45430905 | 17: 46893889-46894340 | 17: 47686864-47687280 |
| 17: 47695080-47695509 | 17: 48154173-48154484 | 17: 48576523-48576943 |
| 17: 48589414-48589865 | 17: 49133108-49133528 | 17: 49964712-49965132 |
| 17: 50118119-50118525 | 17: 50245467-50245868 | 17: 50255738-50256158 |
| 17: 50469054-50469527 | 17: 50481949-50482369 | 17: 50542629-50543049 |
| 17: 50543149-50543544 | 17: 51462010-51462374 | 17: 53757289-53757709 |
| 17: 57410134-57410554 | 17: 57443157-57443577 | 17: 57874417-57874879 |
| 17: 58336459-58336879 | 17: 59870922-59871377 | 17: 60102353-60102773 |
| 17: 60139114-60139534 | 17: 60149415-60149835 | 17: 60596536-60596921 |
| 17: 61251987-61252407 | 17: 61452595-61453055 | 17: 61454172-61454640 |
| 17: 62381519-62381939 | 17: 63700829-63701510 | 17: 63881898-63882318 |
| 17: 63910974-63911394 | 17: 63974188-63974608 | 17: 64020407-64020827 |
| 17: 64500054-64500474 | 17: 64627890-64628296 | 17: 64774034-64774454 |
| 17: 66467037-66467474 | 17: 68198641-68199061 | 17: 68911702-68912007 |
| 17: 72553082-72553499 | 17: 72560502-72560922 | 17: 72660832-72661252 |
| 17: 73298163-73298583 | 17: 73552515-73552935 | 17: 74300759-74301179 |
| 17: 74359929-74360338 | 17: 74374321-74374741 | 17: 74466334-74466798 |
| 17: 74657028-74657394 | 17: 74670908-74671328 | 17: 74706588-74707008 |
| 17: 74730906-74731326 | 17: 74843266-74843721 | 17: 74935478-74935898 |
| 17: 75024863-75025268 | 17: 75087958-75088550 | 17: 75108981-75109401 |
| 17: 75114606-75115026 | 17: 75587595-75588015 | 17: 75646256-75646699 |
| 17: 75844222-75844642 | 17: 75864311-75864731 | 17: 76029408-76029931 |
| 17: 76161698-76162225 | 17: 76173345-76173753 | 17: 76515479-76515899 |
| 17: 76532853-76533286 | 17: 76568716-76569136 | 17: 76791349-76791769 |
| 17: 76870247-76870725 | 17: 77055641-77056061 | 17: 77235532-77236009 |
| 17: 77417497-77417917 | 17: 77610243-77610663 | 17: 77959961-77960381 |
| 17: 78057941-78058351 | 17: 78131377-78131878 | 17: 78159757-78160177 |
| 17: 78358620-78359525 | 17: 78476055-78476475 | 17: 78566396-78566816 |
| 17: 78577106-78577526 | 17: 78883780-78884277 | 17: 78997274-78997872 |
| 17: 79048137-79048557 | 17: 79079755-79080175 | 17: 79178611-79179031 |
| 17: 79393804-79394300 | 17: 79540508-79540924 | 17: 79676449-79676869 |
| 17: 79738836-79739222 | 17: 79790816-79791236 | 17: 79801779-79802199 |
| 17: 79829703-79830084 | 17: 79833455-79833943 | 17: 79948867-79949287 |
| 17: 79978467-79978887 | 17: 79979062-79979573 | 17: 79992800-79993169 |
| 17: 80066891-80067321 | 17: 80067522-80067942 | 17: 80832941-80833572 |
| 17: 80848338-80848757 | 17: 80851458-80851877 | 17: 81148292-81148711 |
| 17: 81341263-81341709 | 17: 81422403-81422823 | 17: 81465869-81466289 |
| 17: 81528658-81529078 | 17: 81653092-81653512 | 17: 81653511-81653931 |
| 17: 81713866-81714286 | 17: 81740455-81740875 | 17: 81832213-81832633 |
| 17: 81846228-81846648 | 17: 81869614-81870034 | 17: 81923378-81923845 |
| 17: 81941602-81942057 | 17: 82035774-82036194 | 17: 82061500-82061974 |
| 17: 82078784-82079769 | 17: 82080978-82081561 | 17: 82083130-82083788 |
| 17: 82087012-82087429 | 17: 82088027-82088447 | 17: 82088750-82089170 |
| 17: 82096170-82096666 | 17: 82100848-82101268 | 17: 82228143-82228647 |
| 17: 82559023-82559676 | 17: 82639433-82639853 | 17: 82967967-82968387 |
| 17: 83031868-83032288 | 18: 1807264-1807684 | 18: 2112773-2113144 |
| 18: 2230614-2231034 | 18: 2851726-2852209 | 18: 3280767-3281181 |
| 18: 3665874-3666355 | 18: 3790819-3791244 | 18: 3874067-3874487 |
| 18: 4130512-4130910 | 18: 5074061-5074473 | 18: 5132991-5133411 |
| 18: 5419590-5420008 | 18: 5518591-5519026 | 18: 5544974-5545394 |
| 18: 6021009-6021399 | 18: 8709978-8710370 | 18: 9069637-9070096 |
| 18: 9433559-9433979 | 18: 9707518-9708050 | 18: 10624036-10624452 |
| 18: 10682192-10682611 | 18: 10832308-10832667 | 18: 10887757-10888081 |
| 18: 10913141-10913560 | 18: 10914625-10915035 | 18: 11033592-11034012 |
| 18: 11055671-11056073 | 18: 11111305-11111720 | 18: 11487913-11488333 |
| 18: 11533389-11533809 | 18: 11534000-11534420 | 18: 11692256-11692676 |
| 18: 11856241-11856629 | 18: 11872753-11873173 | 18: 12038312-12038732 |
| 18: 12067374-12067794 | 18: 12093686-12094106 | 18: 12811439-12811833 |
| 18: 13136164-13136814 | 18: 13826359-13826808 | 18: 15197258-15197803 |
| 18: 22166969-22167389 | 18: 22171098-22171834 | 18: 22324628-22325048 |
| 18: 23114364-23114784 | 18: 23629637-23630047 | 18: 24631521-24631941 |
| 18: 24733860-24734242 | 18: 26547498-26548010 | 18: 29191479-29191899 |
| 18: 37243666-37244204 | 18: 46519070-46519409 | 18: 46679917-46680337 |
| 18: 47248427-47249087 | 18: 47248931-47249351 | 18: 47953377-47953797 |
| 18: 48022330-48022750 | 18: 48137543-48137963 | 18: 48997820-48998239 |
| 18: 50267992-50268874 | 18: 50282015-50282435 | 18: 50340001-50340421 |
| 18: 55866964-55867384 | 18: 55953705-55954125 | 18: 55971247-55971633 |
| 18: 56079600-56079991 | 18: 57439125-57439545 | 18: 57439443-57439863 |
| 18: 57439854-57440601 | 18: 58046977-58047463 | 18: 58128710-58129113 |
| 18: 58180230-58180622 | 18: 58311627-58312047 | 18: 58361429-58361849 |
| 18: 59096071-59096500 | 18: 59264123-59264623 | 18: 59274321-59274741 |
| 18: 59957267-59957640 | 18: 60004008-60004428 | 18: 60010015-60010435 |
| 18: 60149499-60149919 | 18: 60264001-60264419 | 18: 63065120-63065684 |
| 18: 63088921-63089341 | 18: 64207350-64207749 | 18: 69115329-69115749 |
| 18: 69402458-69403047 | 18: 74855802-74856222 | 18: 75203831-75204838 |
| 18: 75531755-75532175 | 18: 75712066-75712486 | 18: 75712432-75712852 |
| 18: 75926721-75927042 | 18: 76350638-76351064 | 18: 76557966-76558386 |
| 18: 76567862-76568282 | 18: 76689863-76690277 | 18: 76955446-76955893 |
| 18: 77251659-77252090 | 18: 77733855-77734275 | 18: 77896927-77897326 |
| 18: 77903242-77903662 | 18: 77975846-77976286 | 18: 77978796-77979216 |
| 18: 78126427-78126847 | 18: 78313169-78313589 | 18: 78430605-78431012 |
| 18: 78503014-78503434 | 18: 78558735-78559155 | 18: 78631814-78632234 |
| 18: 78659090-78659510 | 18: 78726594-78727014 | 18: 78781094-78781514 |
| 18: 79068501-79068921 | 18: 79382746-79383166 | 18: 79491567-79491987 |
| 18: 79579023-79579443 | 18: 79625112-79625678 | 18: 79637355-79637830 |
| 18: 79752266-79752686 | 18: 79772323-79772743 | 18: 79782600-79783020 |
| 18: 79782816-79783529 | 18: 79848521-79849045 | 18: 79877883-79878303 |
| 18: 79878153-79878573 | 18: 80246085-80246505 | 19: 511043-511463 |
| 19: 615584-616156 | 19: 751999-752419 | 19: 755090-755510 |
| 19: 1004238-1004658 | 19: 1004449-1004869 | 19: 1005327-1005747 |
| 19: 1037961-1038381 | 19: 1265708-1266358 | 19: 1274428-1274884 |
| 19: 1290308-1290728 | 19: 1325904-1326366 | 19: 1395519-1395939 |
| 19: 1482369-1482789 | 19: 1800216-1800636 | 19: 1829800-1830209 |
| 19: 1871269-1871722 | 19: 1876511-1877018 | 19: 1908239-1908725 |
| 19: 1942767-1943187 | 19: 2094316-2094789 | 19: 2135468-2135888 |
| 19: 2149629-2150047 | 19: 2160395-2160815 | 19: 2389494-2389914 |
| 19: 2410281-2410701 | 19: 2487777-2488197 | 19: 2714908-2715303 |
| 19: 2796141-2796611 | 19: 2867140-2867529 | 19: 3028063-3028494 |
| 19: 3133397-3133817 | 19: 3162924-3163344 | 19: 3381283-3381703 |
| 19: 3834302-3834722 | 19: 3883230-3883630 | 19: 3938489-3938862 |
| 19: 3978124-3978544 | 19: 3982302-3982756 | 19: 4038702-4039109 |
| 19: 4059921-4060341 | 19: 4062223-4062678 | 19: 4207286-4207706 |
| 19: 4328573-4328993 | 19: 4566203-4566623 | 19: 4567045-4567452 |
| 19: 4576157-4576577 | 19: 4792674-4793227 | 19: 4804349-4804769 |
| 19: 4827716-4828125 | 19: 4908797-4909217 | 19: 4940018-4940438 |
| 19: 5322292-5322712 | 19: 5499133-5499553 | 19: 5582128-5582601 |
| 19: 5585487-5585920 | 19: 5772669-5773089 | 19: 5823622-5824042 |
| 19: 5832127-5832547 | 19: 6258991-6259411 | 19: 6593571-6593991 |
| 19: 6707245-6707665 | 19: 6772244-6772791 | 19: 7004942-7005362 |
| 19: 7483195-7483615 | 19: 7691079-7691499 | 19: 7868263-7868683 |
| 19: 7929341-7929761 | 19: 8089448-8089868 | 19: 8129171-8129591 |
| 19: 8421863-8422310 | 19: 8586710-8587115 | 19: 8589241-8589778 |
| 19: 8596045-8596465 | 19: 8609301-8609721 | 19: 8697652-8698072 |
| 19: 9580826-9581209 | 19: 10287339-10287759 | 19: 10391036-10391456 |
| 19: 10925494-10925914 | 19: 10927532-10927952 | 19: 11538705-11539125 |
| 19: 11865058-11865436 | 19: 12064023-12064443 | 19: 12513491-12514093 |
| 19: 12755537-12755957 | 19: 12843366-12843786 | 19: 13172228-13172694 |
| 19: 13796622-13797042 | 19: 13836390-13836810 | 19: 13840111-13840531 |
| 19: 13877223-13877949 | 19: 13918166-13918586 | 19: 13957130-13957550 |
| 19: 14027788-14028208 | 19: 14333129-14333549 | 19: 14772293-14772713 |
| 19: 14882688-14883107 | 19: 14941319-14941690 | 19: 15006851-15007247 |
| 19: 15304588-15304968 | 19: 15469720-15470140 | 19: 15728197-15728617 |
| 19: 15902163-15902804 | 19: 16081790-16082226 | 19: 16202388-16202813 |
| 19: 16283496-16284022 | 19: 16338851-16339271 | 19: 16862556-16863073 |
| 19: 16888505-16888925 | 19: 17076669-17077089 | 19: 17351889-17352309 |
| 19: 17450176-17450563 | 19: 17788160-17788616 | 19: 17981900-17982320 |
| 19: 18082047-18082467 | 19: 18386919-18387251 | 19: 18432992-18433412 |
| 19: 18447230-18447650 | 19: 18649443-18650100 | 19: 18877977-18878397 |
| 19: 19261200-19261620 | 19: 19462039-19462459 | 19: 19539878-19540443 |
| 19: 20035988-20036388 | 19: 21483116-21483536 | 19: 21483228-21483648 |
| 19: 22215209-22215629 | 19: 22901760-22902180 | 19: 23203810-23204330 |
| 19: 24001595-24002015 | 19: 28484751-28485171 | 19: 29087349-29087769 |
| 19: 29093087-29093507 | 19: 29505901-29506321 | 19: 29529671-29530215 |
| 19: 29538223-29538643 | 19: 29833517-29833937 | 19: 30443374-30443794 |
| 19: 32362813-32363233 | 19: 32676612-32677032 | 19: 32676942-32677379 |
| 19: 32824447-32824867 | 19: 32837915-32838332 | 19: 33235568-33235988 |
| 19: 33771867-33772390 | 19: 33906679-33907350 | 19: 34300319-34300739 |
| 19: 35352014-35352434 | 19: 35358119-35358539 | 19: 35502051-35502471 |
| 19: 35557398-35557818 | 19: 35641509-35642148 | 19: 35860384-35860804 |
| 19: 35979048-35979453 | 19: 36244928-36245515 | 19: 36329357-36329726 |
| 19: 36329981-36330581 | 19: 36791340-36791760 | 19: 38060303-38060724 |
| 19: 38213813-38214233 | 19: 38467480-38467900 | 19: 38468850-38469270 |
| 19: 38468977-38469397 | 19: 38483173-38483593 | 19: 38485918-38486338 |
| 19: 38561465-38561885 | 19: 38617814-38618234 | 19: 38681009-38681429 |
| 19: 38693393-38693892 | 19: 38792739-38793159 | 19: 39247802-39248474 |
| 19: 40810559-40811055 | 19: 40843296-40843766 | 19: 41025744-41026164 |
| 19: 41165096-41165483 | 19: 41428062-41428482 | 19: 41506390-41506764 |
| 19: 41844956-41845372 | 19: 41915719-41916210 | 19: 42199224-42199644 |
| 19: 42280889-42281390 | 19: 42316717-42317137 | 19: 42323452-42324002 |
| 19: 42422972-42423392 | 19: 42596134-42596554 | 19: 42670363-42670783 |
| 19: 42856848-42857268 | 19: 42868634-42869052 | 19: 43083658-43084078 |
| 19: 43774347-43774775 | 19: 44722210-44722630 | 19: 45045931-45046352 |
| 19: 45161246-45161766 | 19: 45187458-45187878 | 19: 45385407-45386048 |
| 19: 45591621-45592041 | 19: 45884744-45885221 | 19: 45952911-45953331 |
| 19: 46829624-46830041 | 19: 47275184-47275661 | 19: 47598519-47599137 |
| 19: 47939348-47939768 | 19: 47949879-47950218 | 19: 47950103-47950523 |
| 19: 48107868-48108288 | 19: 48279611-48280006 | 19: 48405924-48406344 |
| 19: 48415767-48416305 | 19: 48491441-48491861 | 19: 48539383-48539709 |
| 19: 48697987-48698407 | 19: 49036167-49036587 | 19: 49042028-49042448 |
| 19: 49200469-49200935 | 19: 49490739-49491159 | 19: 49613293-49613664 |
| 19: 49745304-49745724 | 19: 49745475-49745895 | 19: 49850044-49850464 |
| 19: 49897388-49897854 | 19: 50436333-50436753 | 19: 50666269-50666876 |
| 19: 50791458-50791878 | 19: 51020082-51020502 | 19: 51372540-51372960 |
| 19: 51417327-51417747 | 19: 51431460-51431877 | 19: 51509227-51509561 |
| 19: 51564280-51564670 | 19: 51601016-51601627 | 19: 51900910-51901330 |
| 19: 53280680-53281100 | 19: 53282662-53283082 | 19: 53290970-53291390 |
| 19: 53336651-53337071 | 19: 53444126-53444527 | 19: 53444671-53445091 |
| 19: 53697357-53697777 | 19: 53775399-53775819 | 19: 53788523-53788943 |
| 19: 54064922-54065340 | 19: 54114005-54114425 | 19: 54164294-54164714 |
| 19: 54279398-54279818 | 19: 54286871-54287285 | 19: 54357454-54357874 |
| 19: 54561852-54562282 | 19: 54575830-54576250 | 19: 55079961-55080421 |
| 19: 55081565-55082751 | 19: 55255494-55255914 | 19: 55302443-55303060 |
| 19: 55545373-55545793 | 19: 55586080-55586500 | 19: 55857891-55858311 |
| 19: 56097273-56097976 | 19: 56253522-56253942 | 19: 56965710-56966130 |
| 19: 57119552-57119972 | 19: 57317516-57317936 | 19: 58049448-58049868 |
| 19: 58052343-58052763 | 19: 58059659-58060171 | 19: 58117833-58118253 |
| 19: 58118040-58118863 | 19: 58346938-58347358 | 19: 58355922-58356342 |
| 2: 284878-285298 | 2: 286879-287299 | 2: 495599-496019 |
| 2: 535091-535400 | 2: 595062-595482 | 2: 637281-637685 |
| 2: 1676290-1676677 | 2: 1676503-1676923 | 2: 1747387-1747793 |
| 2: 2120616-2121036 | 2: 2189771-2190230 | 2: 2294856-2295276 |
| 2: 2567978-2568398 | 2: 2751347-2751767 | 2: 2771990-2772410 |
| 2: 2843931-2844351 | 2: 2867428-2867848 | 2: 2869592-2870012 |
| 2: 3498848-3499276 | 2: 3632637-3633057 | 2: 3786053-3786473 |
| 2: 6926865-6927310 | 2: 7096781-7097201 | 2: 8031750-8032170 |
| 2: 8229398-8229992 | 2: 8583574-8583994 | 2: 9497078-9497469 |
| 2: 9628254-9628658 | 2: 10009894-10010297 | 2: 10029206-10029701 |
| 2: 10075330-10075732 | 2: 10185319-10185739 | 2: 10285437-10285873 |
| 2: 10834938-10835353 | 2: 11505807-11506209 | 2: 11656507-11656928 |
| 2: 11692369-11692775 | 2: 11822581-11823001 | 2: 12169704-12170124 |
| 2: 12451819-12452239 | 2: 12718803-12719244 | 2: 15944032-15944452 |
| 2: 16055998-16056418 | 2: 16224338-16224758 | 2: 18535989-18536409 |
| 2: 19356849-19357850 | 2: 20625863-20626352 | 2: 20665127-20666023 |
| 2: 24791484-24791901 | 2: 24815370-24815856 | 2: 24927009-24927429 |
| 2: 25276719-25277139 | 2: 25294827-25295247 | 2: 25822179-25822599 |
| 2: 25951698-25952107 | 2: 26174065-26174485 | 2: 26769018-26769438 |
| 2: 27309667-27310125 | 2: 28755178-28755597 | 2: 29238724-29239143 |
| 2: 30230410-30231284 | 2: 35871499-35871919 | 2: 36479421-36479841 |
| 2: 36617997-36618417 | 2: 37148689-37149075 | 2: 37653786-37654288 |
| 2: 38073822-38074582 | 2: 38667059-38667479 | 2: 38748431-38748849 |
| 2: 39138974-39139293 | 2: 39448132-39448553 | 2: 44944880-44945311 |
| 2: 44949143-44949563 | 2: 44954391-44955041 | 2: 45000362-45000891 |
| 2: 45778764-45779234 | 2: 46299775-46300195 | 2: 46814237-46814657 |
| 2: 47304862-47305282 | 2: 52571061-52571481 | 2: 52572044-52572463 |
| 2: 52572718-52573138 | 2: 52629699-52630119 | 2: 57553095-57553532 |
| 2: 57699982-57700359 | 2: 58251819-58252230 | 2: 58460222-58460638 |
| 2: 58461783-58462172 | 2: 58479786-58480180 | 2: 59242923-59243329 |
| 2: 63055338-63056247 | 2: 63991644-63992064 | 2: 64636291-64636711 |
| 2: 64672606-64673026 | 2: 64889203-64889581 | 2: 65040062-65040482 |
| 2: 65064862-65065282 | 2: 65411142-65411542 | 2: 66439706-66440126 |
| 2: 66440298-66440718 | 2: 66445181-66445916 | 2: 66446234-66446654 |
| 2: 67277523-67277930 | 2: 68292620-68293063 | 2: 68646026-68646446 |
| 2: 68838692-68839095 | 2: 69014405-69014842 | 2: 69989437-69989857 |
| 2: 69989632-69990052 | 2: 70886269-70886689 | 2: 70888050-70888470 |
| 2: 70889250-70889806 | 2: 70904248-70905190 | 2: 70907109-70907529 |
| 2: 70977986-70978488 | 2: 71465809-71466321 | 2: 71548519-71548939 |
| 2: 72538778-72539114 | 2: 72826135-72826555 | 2: 73739633-73740053 |
| 2: 74414575-74415109 | 2: 74555138-74555558 | 2: 75493232-75493652 |
| 2: 75598133-75598553 | 2: 75800346-75800796 | 2: 77954876-77955296 |
| 2: 81194222-81194642 | 2: 81194674-81195094 | 2: 83877433-83877853 |
| 2: 84077280-84077662 | 2: 84888720-84889140 | 2: 84906874-84907294 |
| 2: 85134569-85135408 | 2: 85470818-85471250 | 2: 85584424-85584941 |
| 2: 86251827-86252215 | 2: 86695129-86695521 | 2: 86809465-86809927 |
| 2: 88115862-88116373 | 2: 88254909-88255313 | 2: 88291051-88291471 |
| 2: 88881275-88881670 | 2: 89610183-89610634 | 2: 89610957-89611377 |
| 2: 89611631-89612260 | 2: 89977254-89977671 | 2: 90238448-90238868 |
| 2: 91748017-91748437 | 2: 91752010-91752430 | 2: 91755818-91756238 |
| 2: 94857715-94858635 | 2: 96115949-96116413 | 2: 96148652-96149072 |
| 2: 96524636-96525056 | 2: 96549910-96550351 | 2: 96773929-96774349 |
| 2: 96839545-96839965 | 2: 97095149-97095646 | 2: 98747929-98748349 |
| 2: 101003929-101004349 | 2: 101117160-101117599 | 2: 101120240-101120660 |
| 2: 101135321-101135741 | 2: 101342465-101342879 | 2: 102006638-102007058 |
| 2: 102231928-102232348 | 2: 104804559-104804979 | 2: 105107015-105107435 |
| 2: 105298400-105298821 | 2: 105399218-105399638 | 2: 106104619-106105039 |
| 2: 106507419-106507839 | 2: 108301506-108301926 | 2: 109170794-109171152 |
| 2: 112253831-112254187 | 2: 112591358-112591766 | 2: 112692491-112692934 |
| 2: 112779376-112779740 | 2: 113275658-113276101 | 2: 113276751-113277250 |
| 2: 113553770-113554190 | 2: 113557594-113558014 | 2: 117997297-117997705 |
| 2: 118847451-118847871 | 2: 119678210-119678642 | 2: 120210370-120210790 |
| 2: 120654184-120654604 | 2: 120955230-120955650 | 2: 121080586-121080994 |
| 2: 121789877-121790239 | 2: 125810971-125811391 | 2: 126557975-126558389 |
| 2: 126665509-126665911 | 2: 126668766-126669186 | 2: 126703394-126703812 |
| 2: 126771720-126772206 | 2: 126877833-126878253 | 2: 126884173-126884593 |
| 2: 127064769-127065219 | 2: 127628138-127628558 | 2: 127654606-127655026 |
| 2: 128690637-128691057 | 2: 129758487-129758907 | 2: 130371761-130372259 |
| 2: 130837596-130838016 | 2: 131040238-131040658 | 2: 131061207-131061627 |
| 2: 131120489-131120909 | 2: 131478490-131478856 | 2: 132671113-132671544 |
| 2: 134411777-134412197 | 2: 135162269-135162689 | 2: 140581230-140581587 |
| 2: 142858408-142858761 | 2: 142936758-142937178 | 2: 146587612-146588032 |
| 2: 149587892-149588312 | 2: 152468855-152469275 | 2: 153498138-153498670 |
| 2: 156319706-156320706 | 2: 156328930-156329350 | 2: 156329200-156329620 |
| 2: 157300595-157301015 | 2: 159147285-159147752 | 2: 161416856-161417276 |
| 2: 161417713-161418133 | 2: 161427229-161427649 | 2: 161427474-161428167 |
| 2: 164773599-164773977 | 2: 165406190-165406527 | 2: 166180836-166181256 |
| 2: 167264441-167264878 | 2: 169694966-169695386 | 2: 169768356-169769227 |
| 2: 170712434-170712854 | 2: 170716788-170717336 | 2: 170717124-170717544 |
| 2: 170845290-170845710 | 2: 171374998-171375440 | 2: 172080379-172080799 |
| 2: 172081038-172081714 | 2: 172093459-172094161 | 2: 174012501-174013324 |
| 2: 174484115-174484499 | 2: 176093597-176094083 | 2: 176151539-176151996 |
| 2: 176159537-176160360 | 2: 176162731-176163151 | 2: 176163016-176163436 |
| 2: 176164853-176165273 | 2: 176171755-176172586 | 2: 176174716-176175349 |
| 2: 177165202-177165622 | 2: 182865952-182866612 | 2: 197255594-197256014 |
| 2: 199848432-199848853 | 2: 199985559-199985964 | 2: 201975208-201975628 |
| 2: 202171287-202171776 | 2: 202179295-202179715 | 2: 205684795-205685389 |
| 2: 205776315-205776735 | 2: 207767179-207767608 | 2: 207767519-207768113 |
| 2: 207770700-207771289 | 2: 208124021-208124665 | 2: 209579280-209579843 |
| 2: 216501099-216501519 | 2: 217360798-217361218 | 2: 218444673-218445093 |
| 2: 218880788-218881208 | 2: 219057243-219057713 | 2: 219331776-219332196 |
| 2: 219332550-219332970 | 2: 222302039-222302459 | 2: 222312116-222312946 |
| 2: 222423736-222424156 | 2: 224835262-224835723 | 2: 225122591-225123011 |
| 2: 225581886-225582306 | 2: 225582677-225583097 | 2: 229335165-229335541 |
| 2: 230814222-230814642 | 2: 230929069-230929474 | 2: 231400745-231401165 |
| 2: 231458364-231458784 | 2: 231642809-231643310 | 2: 231665994-231666414 |
| 2: 231666917-231667372 | 2: 232315502-232315968 | 2: 232452352-232452772 |
| 2: 232457076-232457484 | 2: 232885654-232886074 | 2: 232925314-232925734 |
| 2: 233013047-233013467 | 2: 233170085-233170505 | 2: 234406135-234406632 |
| 2: 235052573-235052993 | 2: 235506828-235507248 | 2: 235754809-235755327 |
| 2: 236048006-236048426 | 2: 236159226-236159985 | 2: 236988656-236989076 |
| 2: 236997888-236998237 | 2: 237072053-237072473 | 2: 237372048-237372468 |
| 2: 237374625-237375045 | 2: 237398144-237398516 | 2: 237445729-237446149 |
| 2: 237540204-237540624 | 2: 237968264-237968684 | 2: 238009337-238009715 |
| 2: 238170388-238170808 | 2: 238259567-238259987 | 2: 238966509-238966929 |
| 2: 239109788-239110194 | 2: 239338141-239338507 | 2: 239358371-239358750 |
| 2: 239482889-239483309 | 2: 239507556-239507976 | 2: 239690560-239690974 |
| 2: 240158176-240158929 | 2: 240624938-240625358 | 2: 241031645-241032110 |
| 2: 241126215-241126734 | 2: 241246610-241247030 | 2: 241432710-241433130 |
| 2: 241462372-241462767 | 2: 241509145-241509614 | 2: 241533064-241533484 |
| 2: 241542194-241542614 | 2: 241678841-241679244 | 2: 241740913-241741333 |
| 2: 241852552-241853060 | 2: 241869471-241869891 | 2: 241971128-241971532 |
| 2: 242003243-242003663 | 2: 242004979-242005346 | 2: 242031566-242031986 |
| 20: 370369-370773 | 20: 609372-609871 | 20: 663217-663637 |
| 20: 1571784-1572204 | 20: 2289620-2290040 | 20: 3682237-3682749 |
| 20: 4289875-4290284 | 20: 4471149-4471516 | 20: 4474820-4475235 |
| 20: 4687314-4687791 | 20: 4869764-4870184 | 20: 4936931-4937351 |
| 20: 5292638-5293101 | 20: 5302168-5302603 | 20: 5347259-5347679 |
| 20: 5470116-5470535 | 20: 5471000-5471420 | 20: 5551074-5551525 |
| 20: 7375411-7375831 | 20: 7697274-7697694 | 20: 10296571-10296991 |
| 20: 11708255-11708675 | 20: 11867699-11868059 | 20: 12379941-12380428 |
| 20: 14352113-14352506 | 20: 17895597-17896017 | 20: 19314604-19315024 |
| 20: 19735373-19735882 | 20: 19794954-19795321 | 20: 20003025-20003445 |
| 20: 20365703-20366123 | 20: 20452233-20452874 | 20: 21019975-21020938 |
| 20: 21104174-21104684 | 20: 21507386-21507939 | 20: 21509365-21509785 |
| 20: 22568178-22568598 | 20: 23040576-23040996 | 20: 23232351-23232771 |
| 20: 24303132-24303656 | 20: 24645152-24645572 | 20: 24832050-24832383 |
| 20: 25339680-25340100 | 20: 25985617-25986037 | 20: 25987066-25987486 |
| 20: 30282334-30282727 | 20: 30287187-30287607 | 20: 30316033-30316453 |
| 20: 30335571-30336071 | 20: 30361813-30362232 | 20: 30362022-30362439 |
| 20: 30894615-30895035 | 20: 31484072-31484492 | 20: 31502760-31503154 |
| 20: 31608140-31608560 | 20: 31826536-31826956 | 20: 31901121-31901541 |
| 20: 32031064-32031484 | 20: 32329911-32330331 | 20: 32538701-32539220 |
| 20: 32539015-32539435 | 20: 32560856-32561326 | 20: 34268763-34269360 |
| 20: 34269231-34269651 | 20: 35528998-35529418 | 20: 35729323-35729743 |
| 20: 36098275-36098695 | 20: 36540937-36541357 | 20: 37411646-37412066 |
| 20: 37845874-37846294 | 20: 37914856-37915336 | 20: 37920223-37920643 |
| 20: 38726174-38726594 | 20: 38727627-38728525 | 20: 38840600-38841020 |
| 20: 40683221-40683713 | 20: 40860355-40860742 | 20: 41409085-41409505 |
| 20: 41426487-41426907 | 20: 41533081-41533468 | 20: 43460368-43460786 |
| 20: 43507004-43507482 | 20: 43750135-43750551 | 20: 44117237-44117657 |
| 20: 44159394-44159814 | 20: 44248071-44248491 | 20: 45097974-45098532 |
| 20: 45640342-45640762 | 20: 46000548-46000968 | 20: 46245878-46246298 |
| 20: 46317159-46317631 | 20: 46550548-46550968 | 20: 46714183-46714603 |
| 20: 46808228-46808598 | 20: 47676491-47676911 | 20: 47698010-47698430 |
| 20: 47713817-47714310 | 20: 47747769-47748189 | 20: 47849084-47849510 |
| 20: 47950812-47951232 | 20: 47956500-47956920 | 20: 47957059-47957475 |
| 20: 47968570-47968990 | 20: 48042057-48042462 | 20: 48153149-48153569 |
| 20: 48192173-48192593 | 20: 48225567-48225987 | 20: 48242542-48242962 |
| 20: 48369148-48369568 | 20: 49303147-49303567 | 20: 49572886-49573306 |
| 20: 49899362-49899743 | 20: 50048341-50048780 | 20: 50330606-50331026 |
| 20: 50959548-50960141 | 20: 51306410-51306830 | 20: 51497873-51498293 |
| 20: 51593604-51593927 | 20: 53660056-53660547 | 20: 53829340-53829760 |
| 20: 53949274-53949752 | 20: 54071659-54072054 | 20: 54612073-54612493 |
| 20: 56249045-56249465 | 20: 56713518-56713938 | 20: 56721645-56722065 |
| 20: 56987199-56987619 | 20: 57473161-57473609 | 20: 57514702-57515122 |
| 20: 57620249-57620669 | 20: 57664383-57664803 | 20: 57679290-57679710 |
| 20: 57697921-57698341 | 20: 57706563-57707056 | 20: 58460289-58460709 |
| 20: 58853074-58853494 | 20: 58853244-58853664 | 20: 59149680-59150100 |
| 20: 59773385-59773805 | 20: 60750211-60750629 | 20: 60807086-60807506 |
| 20: 60920524-60920906 | 20: 60968393-60968973 | 20: 61078919-61079332 |
| 20: 61193199-61193619 | 20: 61281364-61281755 | 20: 61291219-61291615 |
| 20: 61389804-61390224 | 20: 61480111-61480531 | 20: 61484387-61484807 |
| 20: 61519530-61519950 | 20: 61536394-61536814 | 20: 61537375-61537795 |
| 20: 61539601-61540021 | 20: 61544390-61544806 | 20: 61557544-61557946 |
| 20: 61602724-61603139 | 20: 61619685-61620105 | 20: 61686393-61686813 |
| 20: 61745333-61745754 | 20: 61773308-61773728 | 20: 61777203-61777623 |
| 20: 61849850-61850238 | 20: 61873726-61874146 | 20: 61881028-61881448 |
| 20: 61885144-61885564 | 20: 61925294-61925714 | 20: 61997925-61998339 |
| 20: 62002856-62003311 | 20: 62004948-62005381 | 20: 62342940-62343360 |
| 20: 62391048-62391468 | 20: 62516134-62516640 | 20: 62603760-62604180 |
| 20: 62735237-62735657 | 20: 62830817-62831264 | 20: 62838504-62838924 |
| 20: 62987542-62987962 | 20: 63039927-63040411 | 20: 63043473-63043893 |
| 20: 63043738-63044418 | 20: 63123486-63123904 | 20: 63148386-63148806 |
| 20: 63191438-63191858 | 20: 63298754-63299182 | 20: 63328950-63329370 |
| 20: 63341967-63342387 | 20: 63342659-63343145 | 20: 63373857-63374277 |
| 20: 63377724-63378144 | 20: 63400103-63400516 | 20: 63400511-63400931 |
| 20: 63414867-63415287 | 20: 63420553-63420973 | 20: 63427889-63428309 |
| 20: 63465693-63466113 | 20: 63466446-63466866 | 20: 63477296-63477748 |
| 20: 63742707-63743127 | 20: 63751834-63752254 | 20: 63799805-63800225 |
| 20: 63802434-63802852 | 20: 63930977-63931397 | 20: 64053897-64054416 |
| 20: 64164185-64164605 | 20: 64178256-64178676 | 20: 64178782-64179202 |
| 21: 13827236-13827656 | 21: 14011688-14012108 | 21: 14072952-14073372 |
| 21: 17146651-17146957 | 21: 17454592-17455012 | 21: 18294956-18295349 |
| 21: 18752795-18753236 | 21: 18789883-18790294 | 21: 18857618-18858038 |
| 21: 25361697-25362225 | 21: 26138765-26139164 | 21: 26846918-26847356 |
| 21: 27143373-27143793 | 21: 29071131-29071444 | 21: 29196154-29196611 |
| 21: 29297763-29298183 | 21: 31625038-31625458 | 21: 31972722-31973086 |
| 21: 32412976-32413489 | 21: 35026516-35027112 | 21: 35417233-35417572 |
| 21: 36245296-36245716 | 21: 36430071-36430491 | 21: 36509039-36509446 |
| 21: 36696156-36696576 | 21: 36756406-36756802 | 21: 38238099-38238519 |
| 21: 38755578-38755998 | 21: 38766853-38767273 | 21: 39744686-39745089 |
| 21: 41458571-41459016 | 21: 41475695-41476119 | 21: 41481127-41481547 |
| 21: 41509927-41510308 | 21: 41581246-41581937 | 21: 41816546-41816972 |
| 21: 41877777-41878197 | 21: 42305791-42306211 | 21: 42486589-42487009 |
| 21: 42562009-42562369 | 21: 43985674-43986094 | 21: 44067487-44067886 |
| 21: 44293573-44293993 | 21: 44417194-44417972 | 21: 44418314-44418734 |
| 21: 44427807-44428227 | 21: 44446010-44446430 | 21: 44600209-44600595 |
| 21: 44653060-44653480 | 21: 44756303-44756723 | 21: 45093181-45093601 |
| 21: 45293095-45293515 | 21: 45433348-45433720 | 21: 45439885-45440305 |
| 21: 45475065-45475485 | 21: 45501880-45502300 | 21: 45505786-45506204 |
| 21: 45751327-45751740 | 21: 45891997-45892605 | 21: 45922514-45922934 |
| 21: 46002384-46002787 | 21: 46051294-46051714 | 21: 46059445-46059865 |
| 21: 46059949-46060353 | 21: 46604929-46605481 | 22: 11606100-11606520 |
| 22: 11614545-11614965 | 22: 16390771-16391191 | 22: 16582503-16582961 |
| 22: 16615163-16615583 | 22: 16619363-16619765 | 22: 16694390-16694848 |
| 22: 16697202-16697622 | 22: 16697694-16698114 | 22: 16784037-16784457 |
| 22: 17085690-17086190 | 22: 17114513-17114933 | 22: 17558045-17558465 |
| 22: 17896568-17896988 | 22: 19058591-19058989 | 22: 19489433-19489845 |
| 22: 19566288-19566711 | 22: 19577025-19577445 | 22: 19938902-19939322 |
| 22: 19947142-19947562 | 22: 20115504-20115924 | 22: 20165091-20165511 |
| 22: 20259943-20260363 | 22: 21707928-21708348 | 22: 22559488-22559893 |
| 22: 22848725-22849148 | 22: 22928542-22928962 | 22: 23181504-23182066 |
| 22: 23846481-23846901 | 22: 24242998-24243480 | 22: 25402924-25403344 |
| 22: 25777433-25777853 | 22: 25822534-25822954 | 22: 26027250-26027670 |
| 22: 26347209-26347629 | 22: 26347593-26348042 | 22: 26547329-26547749 |
| 22: 26764136-26764524 | 22: 26953129-26953549 | 22: 27124612-27125032 |
| 22: 28810641-28811061 | 22: 29313532-29314192 | 22: 29480720-29481189 |
| 22: 29581781-29582190 | 22: 29730942-29731362 | 22: 30022387-30022807 |
| 22: 31580147-31580567 | 22: 36354882-36355368 | 22: 36361228-36361648 |
| 22: 36369129-36369632 | 22: 36506345-36506765 | 22: 36730502-36730826 |
| 22: 37068878-37069483 | 22: 37207086-37207506 | 22: 37300503-37300923 |
| 22: 37633307-37633727 | 22: 37675517-37675937 | 22: 37943731-37944151 |
| 22: 38051779-38052199 | 22: 38080748-38081171 | 22: 38081660-38082080 |
| 22: 38088632-38089342 | 22: 38397038-38397499 | 22: 38463282-38463773 |
| 22: 39013794-39014214 | 22: 39021232-39021652 | 22: 39254745-39255198 |
| 22: 39388677-39389291 | 22: 39477645-39478065 | 22: 39485104-39485524 |
| 22: 39662536-39662956 | 22: 40183240-40183660 | 22: 40405932-40406352 |
| 22: 40678434-40678847 | 22: 41045178-41045573 | 22: 41217517-41217937 |
| 22: 41412829-41413249 | 22: 41799761-41800234 | 22: 41919931-41920390 |
| 22: 41977256-41977676 | 22: 42340744-42341164 | 22: 42439715-42440160 |
| 22: 42515843-42516251 | 22: 42644646-42645066 | 22: 42857924-42858320 |
| 22: 42963215-42963599 | 22: 43111449-43111951 | 22: 43225428-43225848 |
| 22: 43482099-43482530 | 22: 44438948-44439368 | 22: 44677060-44677480 |
| 22: 44890211-44890631 | 22: 45081449-45081838 | 22: 45129727-45130147 |
| 22: 45309011-45309456 | 22: 45504367-45504982 | 22: 45577453-45577873 |
| 22: 45889509-45889929 | 22: 46036999-46037438 | 22: 46038589-46039009 |
| 22: 46042738-46043128 | 22: 46178867-46179192 | 22: 46693800-46694214 |
| 22: 46709015-46709435 | 22: 46744475-46744812 | 22: 47136864-47137304 |
| 22: 47144063-47144483 | 22: 47631359-47631813 | 22: 47631583-47632003 |
| 22: 48667405-48667825 | 22: 48721127-48721547 | 22: 48890823-48891243 |
| 22: 48899520-48899940 | 22: 48902512-48902932 | 22: 49099148-49099568 |
| 22: 49160893-49161338 | 22: 49168141-49168561 | 22: 49183717-49184137 |
| 22: 49218152-49218572 | 22: 49241614-49242034 | 22: 49301502-49301922 |
| 22: 49306303-49306710 | 22: 49330892-49331312 | 22: 49371433-49371845 |
| 22: 49415672-49416092 | 22: 49416414-49416830 | 22: 49418190-49418610 |
| 22: 49433806-49434224 | 22: 49437837-49438236 | 22: 49461428-49461848 |
| 22: 49520360-49520779 | 22: 49521478-49521898 | 22: 49533964-49534384 |
| 22: 49538978-49539370 | 22: 49540915-49541335 | 22: 49543776-49544147 |
| 22: 49564932-49565352 | 22: 49566783-49567203 | 22: 49571266-49571686 |
| 22: 49573183-49573603 | 22: 49583281-49583701 | 22: 49586288-49586708 |
| 22: 49586450-49586870 | 22: 49590332-49590752 | 22: 49601994-49602414 |
| 22: 49613118-49613538 | 22: 49619834-49620254 | 22: 49631223-49631637 |
| 22: 49712759-49713178 | 22: 49713008-49713388 | 22: 49733582-49733989 |
| 22: 49738524-49738924 | 22: 49930291-49930711 | 22: 49999665-50000347 |
| 22: 50073926-50074346 | 22: 50085354-50086018 | 22: 50091133-50091553 |
| 22: 50469693-50470208 | 22: 50485277-50485697 | 3: 5095697-5096117 |
| 3: 9548817-9549332 | 3: 10199073-10199493 | 3: 10329664-10330107 |
| 3: 10340294-10340714 | 3: 10761550-10761970 | 3: 11989667-11990066 |
| 3: 12986923-12987387 | 3: 13136528-13137015 | 3: 13204017-13204751 |
| 3: 13228046-13228459 | 3: 13637348-13637768 | 3: 13670120-13670540 |
| 3: 13818879-13819299 | 3: 13842881-13843301 | 3: 14403852-14404272 |
| 3: 14915472-14915849 | 3: 15152430-15152752 | 3: 15426475-15426895 |
| 3: 15521477-15521897 | 3: 16372245-16372637 | 3: 16467392-16467812 |
| 3: 16489796-16490211 | 3: 16823692-16824112 | 3: 16883914-16884334 |
| 3: 17872202-17872614 | 3: 18725762-18726183 | 3: 20340750-20341170 |
| 3: 21405893-21406376 | 3: 22381495-22381995 | 3: 23590442-23590783 |
| 3: 23762803-23763223 | 3: 27282062-27282461 | 3: 27768823-27769244 |
| 3: 34361803-34362223 | 3: 36486012-36486451 | 3: 36521381-36521839 |
| 3: 36621208-36621581 | 3: 38134738-38135188 | 3: 38645663-38646061 |
| 3: 38648737-38649157 | 3: 39000479-39000899 | 3: 39408390-39408810 |
| 3: 39455967-39456387 | 3: 40458408-40458801 | 3: 40615451-40616157 |
| 3: 43303047-43303467 | 3: 45760340-45760760 | 3: 45985922-45986298 |
| 3: 46579170-46579536 | 3: 46693957-46694377 | 3: 46731364-46731762 |
| 3: 47877187-47877607 | 3: 48560918-48561464 | 3: 49103417-49103832 |
| 3: 49103750-49104140 | 3: 49118944-49119364 | 3: 49685690-49686471 |
| 3: 49718646-49719109 | 3: 49870034-49870454 | 3: 50066114-50066516 |
| 3: 50106732-50107152 | 3: 50340269-50341008 | 3: 51392441-51392856 |
| 3: 52495149-52495569 | 3: 52609578-52609998 | 3: 52692771-52693191 |
| 3: 52787297-52787717 | 3: 52869648-52869988 | 3: 53046608-53047133 |
| 3: 53523383-53523690 | 3: 53845255-53845644 | 3: 55481946-55482366 |
| 3: 56042899-56043304 | 3: 58149501-58149921 | 3: 58586646-58587231 |
| 3: 58666577-58666960 | 3: 58899434-58899854 | 3: 59050167-59050583 |
| 3: 59308625-59308975 | 3: 59798449-59798833 | 3: 65356640-65357060 |
| 3: 66372835-66373359 | 3: 66400268-66400659 | 3: 66404236-66404656 |
| 3: 66836625-66837016 | 3: 67362670-67363090 | 3: 70120258-70120678 |
| 3: 71544874-71545294 | 3: 71695169-71695549 | 3: 71754722-71755142 |
| 3: 73401057-73401477 | 3: 75214338-75214758 | 3: 75336012-75336413 |
| 3: 75363679-75364099 | 3: 75498577-75498973 | 3: 75506624-75507044 |
| 3: 75590295-75590715 | 3: 75603907-75604327 | 3: 75617077-75617497 |
| 3: 75784493-75784913 | 3: 77097730-77098150 | 3: 82807843-82808285 |
| 3: 100777049-100777421 | 3: 100885598-100886062 | 3: 101516521-101516941 |
| 3: 102904740-102905153 | 3: 105353472-105354091 | 3: 105354023-105354514 |
| 3: 107846519-107846939 | 3: 108953414-108953817 | 3: 109118065-109118485 |
| 3: 109316289-109316709 | 3: 109409783-109410203 | 3: 112185617-112186148 |
| 3: 112304240-112304660 | 3: 112638070-112638490 | 3: 112639377-112639797 |
| 3: 113241996-113242425 | 3: 113271041-113271461 | 3: 113271240-113271660 |
| 3: 114238905-114239326 | 3: 114299487-114299907 | 3: 118851867-118852287 |
| 3: 119038986-119039337 | 3: 119145736-119146199 | 3: 119146257-119146669 |
| 3: 119575791-119576211 | 3: 119635595-119636002 | 3: 119884164-119884480 |
| 3: 120396632-120397052 | 3: 122577371-122577791 | 3: 122777602-122777955 |
| 3: 123337828-123338167 | 3: 123583492-123583811 | 3: 124841241-124841680 |
| 3: 124995289-124995624 | 3: 125141528-125141948 | 3: 125741534-125741954 |
| 3: 125755908-125756328 | 3: 125990027-125990447 | 3: 127172974-127173483 |
| 3: 127775740-127776246 | 3: 128076527-128077080 | 3: 128077023-128077443 |
| 3: 128236867-128237250 | 3: 129003287-129003707 | 3: 129045947-129046428 |
| 3: 129794424-129794844 | 3: 129881647-129882067 | 3: 130013180-130013600 |
| 3: 132694825-132695194 | 3: 133399905-133400325 | 3: 133674088-133674508 |
| 3: 134312886-134313306 | 3: 134783279-134783699 | 3: 135132564-135132954 |
| 3: 135197049-135197456 | 3: 138417904-138418318 | 3: 138714770-138715190 |
| 3: 138943867-138944352 | 3: 139538721-139539143 | 3: 140902436-140902748 |
| 3: 140956506-140956883 | 3: 141076804-141077304 | 3: 141077072-141077492 |
| 3: 141230920-141231390 | 3: 141462784-141463164 | 3: 142439579-142440020 |
| 3: 142827381-142827801 | 3: 143016158-143016578 | 3: 143177076-143177481 |
| 3: 146218588-146218916 | 3: 147396128-147396652 | 3: 147396422-147396842 |
| 3: 147397010-147397430 | 3: 147397957-147398377 | 3: 147409523-147410061 |
| 3: 147410791-147411211 | 3: 147411163-147411583 | 3: 147412398-147412818 |
| 3: 147421453-147421874 | 3: 149507353-149507773 | 3: 150286445-150286806 |
| 3: 150384762-150385182 | 3: 151806468-151806888 | 3: 153371899-153372304 |
| 3: 156605893-156606517 | 3: 156816069-156816726 | 3: 159271970-159272274 |
| 3: 159762546-159762966 | 3: 159852689-159853139 | 3: 159911213-159911633 |
| 3: 160007056-160007382 | 3: 160757097-160757574 | 3: 161224803-161225294 |
| 3: 164004168-164004588 | 3: 167381504-167381924 | 3: 167493743-167494113 |
| 3: 168425823-168426252 | 3: 168519354-168519738 | 3: 169268762-169269093 |
| 3: 169944337-169944728 | 3: 170419197-170420220 | 3: 170467055-170467475 |
| 3: 171028456-171028876 | 3: 172447451-172448018 | 3: 173141101-173141521 |
| 3: 173276069-173276489 | 3: 173529487-173529907 | 3: 177279460-177279839 |
| 3: 177804134-177804460 | 3: 181715548-181715968 | 3: 183161851-183162271 |
| 3: 183983494-183983850 | 3: 184173939-184174423 | 3: 184572586-184573006 |
| 3: 184634016-184634436 | 3: 184769710-184770130 | 3: 185267248-185267658 |
| 3: 185569970-185570390 | 3: 185689271-185689691 | 3: 186065601-186066021 |
| 3: 186801153-186801573 | 3: 186825393-186825914 | 3: 187670118-187670538 |
| 3: 191460756-191461176 | 3: 194056995-194057415 | 3: 194357445-194357865 |
| 3: 194370645-194371094 | 3: 194396800-194397728 | 3: 194397599-194398169 |
| 3: 194398029-194398449 | 3: 195172150-195172570 | 3: 195892885-195893305 |
| 3: 196187020-196187440 | 3: 196337462-196337882 | 3: 196423046-196423465 |
| 3: 196506714-196507111 | 3: 196513856-196514276 | 3: 196685393-196685783 |
| 3: 197426943-197427360 | 3: 197428063-197428483 | 3: 197953870-197954254 |
| 4: 386179-386599 | 4: 622626-623046 | 4: 658942-659433 |
| 4: 680882-681302 | 4: 694504-694920 | 4: 993095-993518 |
| 4: 1003294-1003714 | 4: 1115187-1115607 | 4: 1169647-1170041 |
| 4: 1208960-1209380 | 4: 1210267-1210687 | 4: 1212194-1212614 |
| 4: 1241296-1241716 | 4: 1404524-1404944 | 4: 1405674-1406094 |
| 4: 1541741-1542214 | 4: 1606710-1607130 | 4: 1684838-1685258 |
| 4: 1913036-1913493 | 4: 2393607-2394129 | 4: 2416437-2416857 |
| 4: 2489692-2490090 | 4: 2535229-2535688 | 4: 2923677-2924097 |
| 4: 2977140-2977555 | 4: 3340820-3341240 | 4: 4036711-4037131 |
| 4: 4162563-4162983 | 4: 4170501-4170921 | 4: 4857807-4858409 |
| 4: 4858486-4858906 | 4: 4864804-4865224 | 4: 5274623-5275043 |
| 4: 5399551-5399971 | 4: 5459799-5460199 | 4: 6245459-6245879 |
| 4: 6658496-6659184 | 4: 6664063-6664483 | 4: 6916593-6917013 |
| 4: 6994033-6994453 | 4: 7041102-7041522 | 4: 7046150-7046570 |
| 4: 7055931-7056346 | 4: 7216827-7217247 | 4: 7311383-7311803 |
| 4: 7481658-7482078 | 4: 7482091-7482903 | 4: 7655396-7655816 |
| 4: 7664442-7664862 | 4: 8071808-8072228 | 4: 8290083-8290503 |
| 4: 8586906-8587326 | 4: 8614014-8614434 | 4: 8944391-8944770 |
| 4: 8965649-8966069 | 4: 8990391-8990811 | 4: 9004215-9004635 |
| 4: 9214680-9215100 | 4: 9247902-9248322 | 4: 9248435-9248855 |
| 4: 9453480-9453900 | 4: 9453736-9454142 | 4: 9492574-9492994 |
| 4: 9509577-9509982 | 4: 9521673-9522092 | 4: 9533389-9533809 |
| 4: 9547905-9548279 | 4: 9700100-9700520 | 4: 9750751-9751163 |
| 4: 9760273-9760693 | 4: 9782243-9782663 | 4: 10021308-10021728 |
| 4: 10461656-10462147 | 4: 13522834-13523710 | 4: 13524780-13525361 |
| 4: 13529421-13529886 | 4: 13928057-13928477 | 4: 15703022-15703460 |
| 4: 15984066-15984486 | 4: 16360542-16360962 | 4: 16360811-16361376 |
| 4: 17816325-17816745 | 4: 18489303-18489723 | 4: 19455377-19455797 |
| 4: 23154730-23155147 | 4: 23297336-23297694 | 4: 23888625-23889077 |
| 4: 24674020-24674440 | 4: 25088335-25088802 | 4: 25607590-25607996 |
| 4: 26061236-26061645 | 4: 26124571-26125108 | 4: 30722766-30723186 |
| 4: 36196852-36197272 | 4: 36320097-36320500 | 4: 37648555-37648975 |
| 4: 38335489-38335990 | 4: 38914821-38915241 | 4: 40840485-40840858 |
| 4: 40842068-40842488 | 4: 41256207-41256669 | 4: 41537998-41538418 |
| 4: 41644097-41644563 | 4: 41747588-41748008 | 4: 41752648-41753068 |
| 4: 41866813-41867233 | 4: 46908983-46909424 | 4: 47425572-47425992 |
| 4: 47425840-47426258 | 4: 47467522-47467902 | 4: 48483735-48484656 |
| 4: 48490743-48491165 | 4: 52076726-52077146 | 4: 52370162-52370558 |
| 4: 54108246-54108666 | 4: 54109193-54109613 | 4: 55339864-55340243 |
| 4: 55793309-55793729 | 4: 70811705-70812105 | 4: 70836707-70837027 |
| 4: 73947765-73948173 | 4: 76548655-76549077 | 4: 76948337-76948757 |
| 4: 77605165-77605585 | 4: 77636364-77636784 | 4: 79325756-79326176 |
| 4: 79408063-79408483 | 4: 80183389-80183809 | 4: 80267948-80268368 |
| 4: 84483709-84484129 | 4: 85726543-85726983 | 4: 86032720-86033177 |
| 4: 86961597-86962020 | 4: 88457609-88458158 | 4: 88880833-88881249 |
| 4: 94285520-94285910 | 4: 95539012-95539432 | 4: 99625821-99626210 |
| 4: 99651586-99652006 | 4: 99651825-99652245 | 4: 99653293-99653713 |
| 4: 105145319-105145739 | 4: 106368594-106368978 | 4: 107068787-107069281 |
| 4: 110520045-110520465 | 4: 110618515-110618935 | 4: 112511057-112511820 |
| 4: 113341556-113341914 | 4: 116598368-116598788 | 4: 116598881-116599301 |
| 4: 118492710-118493130 | 4: 118540147-118540567 | 4: 118572845-118573265 |
| 4: 120763047-120763460 | 4: 123811897-123812317 | 4: 127470313-127471005 |
| 4: 131977265-131977671 | 4: 132344983-132345403 | 4: 134944160-134944580 |
| 4: 136355896-136356316 | 4: 138092129-138092521 | 4: 139514562-139514982 |
| 4: 139633114-139633534 | 4: 140498244-140498664 | 4: 142817421-142817845 |
| 4: 143699572-143699992 | 4: 146647350-146647864 | 4: 147761582-147761989 |
| 4: 150232172-150232556 | 4: 153340378-153340730 | 4: 153649713-153650058 |
| 4: 153729156-153729565 | 4: 153781158-153781579 | 4: 154489289-154489709 |
| 4: 154907641-154908114 | 4: 155665333-155665725 | 4: 158572150-158572549 |
| 4: 158861480-158861901 | 4: 164188249-164188669 | 4: 168515450-168515849 |
| 4: 169266156-169266576 | 4: 169774631-169775075 | 4: 172155850-172156262 |
| 4: 173500047-173500543 | 4: 173500696-173501116 | 4: 173522773-173523461 |
| 4: 173526979-173527399 | 4: 173528246-173528666 | 4: 173531338-173531758 |
| 4: 174729435-174729739 | 4: 175472655-175473072 | 4: 175954138-175954532 |
| 4: 179404090-179404498 | 4: 183097739-183098317 | 4: 183275571-183275919 |
| 4: 183912422-183912842 | 4: 184056986-184057406 | 4: 184283975-184284395 |
| 4: 184803131-184803724 | 4: 185150063-185150483 | 4: 185514138-185514558 |
| 4: 185652512-185652930 | 4: 185820083-185820503 | 4: 186698181-186698571 |
| 4: 187995677-187996097 | 4: 188631208-188631628 | 4: 188659394-188659814 |
| 4: 188904480-188904900 | 4: 189472766-189473171 | 4: 189713580-189713973 |
| 4: 189731265-189731684 | 4: 190018542-190018962 | 5: 139989-140409 |
| 5: 322101-322556 | 5: 407049-407388 | 5: 491559-491979 |
| 5: 571796-572216 | 5: 663934-664354 | 5: 1190920-1191340 |
| 5: 1201753-1202173 | 5: 1630981-1631440 | 5: 1701952-1702372 |
| 5: 1729672-1730092 | 5: 1798614-1799034 | 5: 1878589-1879034 |
| 5: 2136787-2137273 | 5: 3026260-3026680 | 5: 3050928-3051348 |
| 5: 3187991-3188411 | 5: 3225614-3226034 | 5: 3535507-3535993 |
| 5: 3599338-3600011 | 5: 3883134-3883554 | 5: 3961982-3962402 |
| 5: 4866064-4866484 | 5: 5034039-5034435 | 5: 6583338-6583844 |
| 5: 7851222-7851719 | 5: 10073332-10073747 | 5: 10262343-10262762 |
| 5: 10776061-10776448 | 5: 11588741-11589161 | 5: 12794935-12795355 |
| 5: 12795207-12795627 | 5: 13479278-13479607 | 5: 14364629-14365032 |
| 5: 14615189-14615609 | 5: 16759465-16759885 | 5: 17654327-17654747 |
| 5: 18393438-18393878 | 5: 21882383-21882762 | 5: 25190729-25191149 |
| 5: 25909112-25909532 | 5: 26326958-26327378 | 5: 27121229-27121643 |
| 5: 27291663-27292083 | 5: 30494339-30494759 | 5: 31273396-31273806 |
| 5: 31808069-31808489 | 5: 33444610-33444969 | 5: 33984458-33984827 |
| 5: 34101972-34102392 | 5: 36526783-36527203 | 5: 38660535-38660866 |
| 5: 39055944-39056257 | 5: 40680885-40682194 | 5: 41057980-41058378 |
| 5: 41790240-41790659 | 5: 42819241-42819661 | 5: 42834822-42835231 |
| 5: 42949528-42950230 | 5: 43609264-43609684 | 5: 44788930-44789350 |
| 5: 55227751-55228279 | 5: 55530565-55530985 | 5: 55976119-55976436 |
| 5: 59617275-59617725 | 5: 62722381-62722801 | 5: 63960792-63961212 |
| 5: 63961004-63961424 | 5: 65163689-65164109 | 5: 66828404-66828824 |
| 5: 71718941-71719483 | 5: 73311797-73312217 | 5: 73380143-73380563 |
| 5: 73444483-73445104 | 5: 74433447-74433874 | 5: 76953034-76953459 |
| 5: 77638578-77639621 | 5: 77957515-77957935 | 5: 79111536-79112000 |
| 5: 79563922-79564342 | 5: 79706309-79706704 | 5: 80082385-80082793 |
| 5: 82356991-82357411 | 5: 85647575-85647969 | 5: 86570216-86570636 |
| 5: 88143351-88143771 | 5: 88189759-88190171 | 5: 88190077-88190465 |
| 5: 88602533-88602953 | 5: 88672534-88673078 | 5: 88689519-88690061 |
| 5: 91222127-91222527 | 5: 91226315-91226735 | 5: 91373595-91374015 |
| 5: 92939411-92939831 | 5: 93570384-93571123 | 5: 93579005-93579671 |
| 5: 93587767-93588252 | 5: 93588234-93588654 | 5: 93594778-93595312 |
| 5: 93596070-93596490 | 5: 93596238-93596736 | 5: 95583818-95584200 |
| 5: 95718218-95718638 | 5: 95759050-95759441 | 5: 95811391-95811811 |
| 5: 96432963-96433383 | 5: 100051762-100052182 | 5: 100052849-100053269 |
| 5: 100053890-100054310 | 5: 100657816-100658236 | 5: 100698270-100698674 |
| 5: 100774780-100775200 | 5: 100905776-100906196 | 5: 102498503-102498923 |
| 5: 103544516-103545006 | 5: 106875488-106875908 | 5: 107478252-107478631 |
| 5: 111091387-111091807 | 5: 111142695-111143115 | 5: 111932609-111933008 |
| 5: 112737338-112737758 | 5: 112862152-112862597 | 5: 113212215-113212642 |
| 5: 115558884-115559204 | 5: 115961618-115962519 | 5: 115963236-115963656 |
| 5: 115963578-115964038 | 5: 121851658-121852078 | 5: 122159867-122160287 |
| 5: 123094260-123094680 | 5: 123094573-123095024 | 5: 123098318-123098738 |
| 5: 124735237-124735657 | 5: 124744296-124744716 | 5: 126600187-126600607 |
| 5: 126869140-126869599 | 5: 127515966-127516386 | 5: 127925831-127926251 |
| 5: 131173087-131173507 | 5: 132655686-132656120 | 5: 132814282-132814702 |
| 5: 132819422-132819886 | 5: 134113492-134113912 | 5: 135027204-135027624 |
| 5: 135034550-135034970 | 5: 135135885-135136314 | 5: 135272097-135272517 |
| 5: 135399478-135400005 | 5: 135578292-135578712 | 5: 136132042-136132462 |
| 5: 138034684-138035032 | 5: 138230838-138231226 | 5: 138274181-138274601 |
| 5: 138439336-138439756 | 5: 139393626-139394547 | 5: 139481403-139481823 |
| 5: 139482395-139482815 | 5: 139492918-139493338 | 5: 139543303-139543784 |
| 5: 139660837-139661649 | 5: 139968388-139968808 | 5: 140927247-140927667 |
| 5: 141343738-141344442 | 5: 141408142-141408562 | 5: 141420681-141421325 |
| 5: 141421212-141421674 | 5: 141513352-141513772 | 5: 141513906-141514540 |
| 5: 141557584-141557937 | 5: 141638104-141638541 | 5: 141753501-141753921 |
| 5: 141811630-141812050 | 5: 141919267-141919687 | 5: 145825981-145826343 |
| 5: 146706379-146706799 | 5: 146707373-146707788 | 5: 149317615-149318035 |
| 5: 149601137-149601490 | 5: 150239193-150239613 | 5: 150412304-150412797 |
| 5: 151709217-151709636 | 5: 151745239-151745680 | 5: 152404502-152404922 |
| 5: 154473726-154474259 | 5: 154631749-154632169 | 5: 157216831-157217217 |
| 5: 157574411-157574865 | 5: 157762541-157762958 | 5: 158023233-158023653 |
| 5: 159091033-159091423 | 5: 159097172-159097877 | 5: 159972631-159973051 |
| 5: 160008229-160008577 | 5: 160606765-160607185 | 5: 162067470-162067890 |
| 5: 163318875-163319295 | 5: 165348660-165349077 | 5: 167309893-167310262 |
| 5: 167688821-167689319 | 5: 167945407-167945827 | 5: 168528547-168528984 |
| 5: 171316025-171316549 | 5: 171450567-171450987 | 5: 172107562-172108058 |
| 5: 172319962-172320288 | 5: 172462358-172462778 | 5: 172659981-172660401 |
| 5: 172772708-172773128 | 5: 172852633-172853074 | 5: 173233619-173234382 |
| 5: 173236533-173237028 | 5: 173236810-173237230 | 5: 173242930-173243350 |
| 5: 173323994-173324423 | 5: 173326507-173326978 | 5: 173819082-173819482 |
| 5: 174312549-174312969 | 5: 175701005-175701409 | 5: 176533503-176533923 |
| 5: 176661346-176661791 | 5: 176875712-176876132 | 5: 177509670-177510118 |
| 5: 178283086-178283555 | 5: 178483607-178484027 | 5: 178973905-178974325 |
| 5: 178982938-178983341 | 5: 179132931-179133351 | 5: 179135875-179136295 |
| 5: 179160783-179161202 | 5: 179167447-179167867 | 5: 179207413-179207833 |
| 5: 179216201-179216621 | 5: 179239533-179239953 | 5: 179289611-179290031 |
| 5: 179363191-179363571 | 5: 179374247-179374667 | 5: 179381744-179382164 |
| 5: 179428174-179428594 | 5: 179456347-179456767 | 5: 179519750-179520170 |
| 5: 179529663-179530083 | 5: 179801654-179802255 | 5: 180113963-180114383 |
| 5: 180328215-180328635 | 5: 181169854-181170274 | 5: 181173949-181174541 |
| 5: 181258763-181259167 | 6: 1605302-1605722 | 6: 2966258-2966678 |
| 6: 4319056-4319474 | 6: 4610241-4610661 | 6: 5068300-5068724 |
| 6: 5086187-5086833 | 6: 5422443-5422862 | 6: 5994527-5995053 |
| 6: 5998877-5999745 | 6: 6001905-6002325 | 6: 6942834-6943307 |
| 6: 10213783-10214193 | 6: 10384603-10385269 | 6: 10882446-10883090 |
| 6: 10883433-10884162 | 6: 11044703-11045123 | 6: 11354008-11354428 |
| 6: 12514162-12514582 | 6: 14309406-14309826 | 6: 15515862-15516379 |
| 6: 16133608-16134027 | 6: 16320155-16320575 | 6: 16700810-16701253 |
| 6: 17015949-17016406 | 6: 19210873-19211293 | 6: 20334220-20334640 |
| 6: 21663719-21664139 | 6: 22147120-22147574 | 6: 23176850-23177270 |
| 6: 24489391-24489810 | 6: 24884392-24884700 | 6: 24910139-24910651 |
| 6: 26522111-26522601 | 6: 26597806-26598226 | 6: 27139059-27139765 |
| 6: 27260242-27260662 | 6: 27545431-27545851 | 6: 27680363-27680783 |
| 6: 27757376-27757796 | 6: 27890236-27890656 | 6: 28090855-28091275 |
| 6: 28208151-28208571 | 6: 28259051-28259736 | 6: 28536926-28537417 |
| 6: 28616418-28616838 | 6: 28729995-28730415 | 6: 29101057-29101460 |
| 6: 29440023-29440443 | 6: 29553077-29553497 | 6: 29850130-29850714 |
| 6: 30127354-30128019 | 6: 30172136-30172636 | 6: 30769224-30769644 |
| 6: 31548064-31548484 | 6: 31641150-31641536 | 6: 31773010-31773423 |
| 6: 31880096-31880509 | 6: 31970703-31971151 | 6: 32047727-32048147 |
| 6: 32069607-32070027 | 6: 32148152-32148606 | 6: 32148988-32149408 |
| 6: 32150275-32150695 | 6: 32220310-32220730 | 6: 32976416-32976836 |
| 6: 32980415-32980835 | 6: 33202842-33203212 | 6: 33593220-33593640 |
| 6: 33975678-33976467 | 6: 33987560-33988034 | 6: 34028191-34028611 |
| 6: 34235002-34235422 | 6: 34424579-34424978 | 6: 34514447-34514867 |
| 6: 34544136-34544546 | 6: 34556350-34556725 | 6: 34810912-34811316 |
| 6: 35509312-35509732 | 6: 35805418-35805838 | 6: 36285073-36285595 |
| 6: 36827402-36827913 | 6: 36849114-36849577 | 6: 37705633-37706083 |
| 6: 39048989-39049450 | 6: 39303593-39304013 | 6: 39431226-39431646 |
| 6: 40164589-40165009 | 6: 41027504-41027924 | 6: 41214722-41215142 |
| 6: 41560336-41560758 | 6: 42010328-42010748 | 6: 42136692-42137112 |
| 6: 43263046-43263466 | 6: 43454264-43454684 | 6: 43725178-43725598 |
| 6: 43924251-43924671 | 6: 44058485-44058980 | 6: 44286006-44286426 |
| 6: 45741802-45742222 | 6: 46655765-46656181 | 6: 50817409-50817886 |
| 6: 50842852-50843485 | 6: 52598134-52598554 | 6: 52610571-52610991 |
| 6: 56431551-56431964 | 6: 63229931-63230414 | 6: 64469425-64469845 |
| 6: 65787974-65788381 | 6: 66094586-66095006 | 6: 71051098-71051518 |
| 6: 71127043-71127459 | 6: 71585036-71585456 | 6: 73225650-73226104 |
| 6: 84967816-84968232 | 6: 88275667-88276145 | 6: 98827246-98827666 |
| 6: 98842615-98843045 | 6: 99604598-99605018 | 6: 99607549-99607969 |
| 6: 99612843-99613350 | 6: 100099915-100100352 | 6: 100135909-100136329 |
| 6: 100447841-100448397 | 6: 100448820-100449240 | 6: 100449083-100449878 |
| 6: 100453359-100453779 | 6: 100455694-100456114 | 6: 100457353-100457773 |
| 6: 100467709-100468129 | 6: 100469180-100469604 | 6: 105795938-105796358 |
| 6: 105880069-105880604 | 6: 106572234-106572654 | 6: 107634634-107635054 |
| 6: 107728713-107729108 | 6: 108167001-108167635 | 6: 108170897-108171317 |
| 6: 108176291-108176920 | 6: 108843997-108844417 | 6: 109289460-109289880 |
| 6: 109465767-109466187 | 6: 110054496-110054916 | 6: 110892564-110892974 |
| 6: 110951958-110952378 | 6: 111978438-111978810 | 6: 112038824-112039244 |
| 6: 112320735-112321155 | 6: 113120635-113121055 | 6: 113564698-113565294 |
| 6: 117263383-117263803 | 6: 117547709-117548198 | 6: 122547702-122548122 |
| 6: 122744561-122744981 | 6: 126643450-126643870 | 6: 126643750-126644170 |
| 6: 127939013-127939423 | 6: 132624225-132624621 | 6: 133746471-133746891 |
| 6: 133836999-133837419 | 6: 134200274-134200648 | 6: 135093409-135093829 |
| 6: 135146391-135146782 | 6: 136269075-136269495 | 6: 137094823-137095204 |
| 6: 137487573-137488069 | 6: 137492265-137492708 | 6: 137494128-137494796 |
| 6: 137495679-137496274 | 6: 138499308-138499728 | 6: 139029791-139030211 |
| 6: 139438768-139439083 | 6: 140009193-140009589 | 6: 140193700-140194120 |
| 6: 142088457-142088877 | 6: 144375002-144375401 | 6: 146027578-146027998 |
| 6: 146029052-146029472 | 6: 146542873-146543293 | 6: 146785375-146785825 |
| 6: 146913819-146914450 | 6: 148682225-148682645 | 6: 149026592-149026982 |
| 6: 149451355-149451839 | 6: 149481688-149482180 | 6: 149856622-149857042 |
| 6: 149860728-149861195 | 6: 149862162-149862701 | 6: 149954906-149955326 |
| 6: 150054880-150055300 | 6: 150119133-150119553 | 6: 151319595-151320015 |
| 6: 152124145-152124599 | 6: 152409645-152410061 | 6: 154080841-154081243 |
| 6: 154676052-154676466 | 6: 155381202-155381594 | 6: 155408971-155409350 |
| 6: 155432429-155432770 | 6: 155456582-155456976 | 6: 155491681-155492046 |
| 6: 155781660-155782054 | 6: 155832302-155832764 | 6: 155873134-155873623 |
| 6: 156028281-156028721 | 6: 156661912-156662332 | 6: 156822911-156823327 |
| 6: 157822371-157822798 | 6: 157982928-157983327 | 6: 158339159-158339581 |
| 6: 158542304-158542816 | 6: 158628587-158629007 | 6: 158908113-158908533 |
| 6: 158939404-158939824 | 6: 159131240-159131660 | 6: 159197163-159197568 |
| 6: 159218074-159218469 | 6: 159232137-159232557 | 6: 159232543-159232961 |
| 6: 159525987-159526407 | 6: 160079506-160079926 | 6: 160837896-160838289 |
| 6: 160863874-160864294 | 6: 161170711-161171112 | 6: 161171038-161171362 |
| 6: 163100360-163100780 | 6: 163340566-163340986 | 6: 163494064-163494484 |
| 6: 166306872-166307351 | 6: 166435629-166436049 | 6: 167350177-167350597 |
| 6: 167832129-167832543 | 6: 167975421-167975843 | 6: 168052356-168052776 |
| 6: 169229359-169229779 | 6: 170043066-170043486 | 6: 170053125-170053545 |
| 6: 170297054-170297474 | 6: 170455643-170456063 | 6: 170469111-170469522 |
| 6: 170471180-170471575 | 7: 289143-289705 | 7: 289491-289911 |
| 7: 422588-423008 | 7: 641096-641517 | 7: 832268-832688 |
| 7: 881205-881625 | 7: 919926-920346 | 7: 936934-937354 |
| 7: 937447-937867 | 7: 941820-942235 | 7: 987229-987649 |
| 7: 1060259-1060679 | 7: 1094164-1094555 | 7: 1238857-1239277 |
| 7: 1312195-1312615 | 7: 1352843-1353263 | 7: 1513300-1513743 |
| 7: 1534374-1534793 | 7: 1542776-1543304 | 7: 1645149-1645569 |
| 7: 1677317-1677766 | 7: 1678696-1679116 | 7: 1735972-1736392 |
| 7: 1922063-1922483 | 7: 2163941-2164298 | 7: 2193138-2193558 |
| 7: 2509159-2509579 | 7: 2734772-2735192 | 7: 2857569-2857986 |
| 7: 2894642-2895062 | 7: 2904242-2904661 | 7: 2922290-2922710 |
| 7: 3094860-3095280 | 7: 4712310-4712773 | 7: 5356464-5356884 |
| 7: 5356922-5357329 | 7: 5531575-5531995 | 7: 5606251-5606671 |
| 7: 5781696-5782116 | 7: 5781910-5782330 | 7: 6050965-6051312 |
| 7: 6284194-6284587 | 7: 6392754-6393174 | 7: 6419193-6419613 |
| 7: 6588944-6589365 | 7: 6871102-6871522 | 7: 9726153-9726573 |
| 7: 9726483-9726903 | 7: 9727092-9727494 | 7: 9728047-9728450 |
| 7: 10476224-10476644 | 7: 10476431-10476851 | 7: 10779527-10779947 |
| 7: 10779721-10780141 | 7: 12571117-12571537 | 7: 18027390-18027810 |
| 7: 18082069-18082489 | 7: 19111473-19111893 | 7: 19119649-19120097 |
| 7: 20539117-20539571 | 7: 20795922-20796342 | 7: 20798856-20799276 |
| 7: 21507867-21508287 | 7: 23205788-23206385 | 7: 23653735-23654155 |
| 7: 24821403-24821843 | 7: 25163307-25163727 | 7: 25858771-25859234 |
| 7: 26153249-26153669 | 7: 27088115-27088619 | 7: 27088468-27088888 |
| 7: 27154932-27155352 | 7: 27201391-27201811 | 7: 28955506-28955926 |
| 7: 30136139-30136799 | 7: 30778045-30778375 | 7: 30911614-30912034 |
| 7: 31336084-31336738 | 7: 31899869-31900198 | 7: 32761800-32762417 |
| 7: 35204203-35204623 | 7: 36078832-36079252 | 7: 36986073-36986493 |
| 7: 37848753-37849173 | 7: 38186002-38186422 | 7: 41045087-41045507 |
| 7: 41918569-41919016 | 7: 43419675-43420095 | 7: 43447370-43447770 |
| 7: 44140503-44140923 | 7: 44145460-44145880 | 7: 44190069-44190488 |
| 7: 44519416-44519836 | 7: 45108798-45109218 | 7: 45575392-45575812 |
| 7: 47530060-47530480 | 7: 48006393-48006813 | 7: 48048287-48048707 |
| 7: 48455154-48455574 | 7: 48455389-48455803 | 7: 48537479-48537821 |
| 7: 48924484-48924973 | 7: 49614958-49615333 | 7: 50275612-50276134 |
| 7: 50327425-50327850 | 7: 51013458-51013878 | 7: 51140114-51140534 |
| 7: 53035666-53036309 | 7: 53186985-53187405 | 7: 55018248-55018679 |
| 7: 55573943-55574363 | 7: 56163018-56163438 | 7: 56287819-56288239 |
| 7: 56569195-56569574 | 7: 56810205-56810625 | 7: 56957692-56958112 |
| 7: 57012887-57013307 | 7: 57179772-57180192 | 7: 57437445-57437865 |
| 7: 57437631-57438051 | 7: 57652750-57653171 | 7: 63348765-63349188 |
| 7: 63572024-63572444 | 7: 64192247-64192667 | 7: 64870221-64870636 |
| 7: 65070770-65071181 | 7: 66503058-66503478 | 7: 66557178-66557598 |
| 7: 70353807-70354213 | 7: 72184009-72184429 | 7: 72882541-72882955 |
| 7: 72948804-72949224 | 7: 73369081-73369501 | 7: 73424215-73424635 |
| 7: 73587911-73588331 | 7: 73766621-73767041 | 7: 73978653-73979073 |
| 7: 74029255-74029870 | 7: 74093896-74094316 | 7: 74252853-74253273 |
| 7: 74258341-74258761 | 7: 74306305-74306920 | 7: 74317382-74317802 |
| 7: 74587389-74587827 | 7: 76285374-76285794 | 7: 76482188-76482738 |
| 7: 77498577-77498977 | 7: 77516293-77516692 | 7: 79344576-79345044 |
| 7: 79451122-79451545 | 7: 79455807-79456177 | 7: 81112158-81112578 |
| 7: 83749419-83749839 | 7: 86786304-86786724 | 7: 88218629-88219194 |
| 7: 91265792-91266212 | 7: 92473774-92474194 | 7: 95318651-95319007 |
| 7: 95527806-95528226 | 7: 96710032-96710452 | 7: 96997686-96998168 |
| 7: 98212471-98212948 | 7: 98226300-98226800 | 7: 98240168-98240606 |
| 7: 98252662-98253082 | 7: 98252840-98253260 | 7: 98614152-98614572 |
| 7: 99041917-99042337 | 7: 99379140-99379560 | 7: 99393643-99394063 |
| 7: 99393781-99394201 | 7: 99415426-99415846 | 7: 99563645-99564065 |
| 7: 100337332-100337752 | 7: 100677247-100677667 | 7: 100678259-100678707 |
| 7: 100840737-100841122 | 7: 100865728-100866148 | 7: 100866290-100866710 |
| 7: 100867883-100868303 | 7: 100965547-100965967 | 7: 101202174-101202594 |
| 7: 101691886-101692306 | 7: 102285184-102285604 | 7: 102366875-102367295 |
| 7: 102749851-102750271 | 7: 102912860-102913322 | 7: 105678745-105679165 |
| 7: 105679082-105679523 | 7: 105827411-105827831 | 7: 106014900-106015320 |
| 7: 107748832-107749252 | 7: 116858514-116858852 | 7: 117157648-117158115 |
| 7: 117791596-117792016 | 7: 118213854-118214393 | 7: 118214529-118214949 |
| 7: 124764117-124764653 | 7: 127702279-127702699 | 7: 128031791-128032211 |
| 7: 128032486-128032914 | 7: 128168546-128168966 | 7: 128271871-128272291 |
| 7: 128351203-128351623 | 7: 128833526-128833946 | 7: 128910457-128910877 |
| 7: 130291878-130292298 | 7: 130304294-130304714 | 7: 130344240-130344660 |
| 7: 130485727-130486146 | 7: 131424617-131424924 | 7: 133170319-133170739 |
| 7: 133945483-133945903 | 7: 135254216-135254636 | 7: 139544197-139544617 |
| 7: 139560664-139561084 | 7: 140023368-140023869 | 7: 140023972-140024350 |
| 7: 140229638-140230144 | 7: 140641083-140641556 | 7: 140646990-140647477 |
| 7: 141918574-141918969 | 7: 141973762-141974182 | 7: 142246941-142247361 |
| 7: 142292620-142292957 | 7: 148528046-148528431 | 7: 149154036-149154456 |
| 7: 149490174-149490579 | 7: 149674690-149675110 | 7: 149720595-149720997 |
| 7: 149790067-149790487 | 7: 150357335-150357755 | 7: 150450675-150451095 |
| 7: 150716360-150716780 | 7: 150947226-150947790 | 7: 151173633-151174047 |
| 7: 151173845-151174265 | 7: 151782754-151783405 | 7: 152143860-152144247 |
| 7: 152707480-152707877 | 7: 152893855-152894275 | 7: 153412288-153412728 |
| 7: 153412914-153413335 | 7: 153413523-153413943 | 7: 154750368-154750788 |
| 7: 154751570-154751987 | 7: 154789632-154790030 | 7: 155184952-155185372 |
| 7: 155198306-155198726 | 7: 155371359-155371779 | 7: 155456075-155456495 |
| 7: 155459852-155460272 | 7: 155462199-155462619 | 7: 156642605-156643032 |
| 7: 156948861-156949312 | 7: 157120169-157120589 | 7: 157124759-157125179 |
| 7: 157521890-157522304 | 7: 157621156-157621597 | 7: 157686021-157686498 |
| 7: 157687780-157688200 | 7: 157693757-157694177 | 7: 158237692-158238088 |
| 7: 158317728-158318284 | 7: 158528121-158528568 | 7: 158588092-158588512 |
| 7: 158590365-158590783 | 7: 158740975-158741395 | 7: 159064112-159064532 |
| 7: 159201653-159202067 | 8: 409640-410060 | 8: 721463-721883 |
| 8: 1097939-1098359 | 8: 1182809-1183224 | 8: 1755655-1756075 |
| 8: 2134107-2134579 | 8: 2137131-2137541 | 8: 2140431-2140951 |
| 8: 2178987-2179550 | 8: 2184941-2185361 | 8: 2251765-2252185 |
| 8: 3182973-3183393 | 8: 3183214-3183634 | 8: 6036913-6037328 |
| 8: 6780390-6780810 | 8: 7065668-7066088 | 8: 10260276-10260665 |
| 8: 10589716-10590136 | 8: 10729349-10729769 | 8: 11287011-11287390 |
| 8: 11628071-11628491 | 8: 11707529-11708018 | 8: 11708034-11708454 |
| 8: 11708243-11708663 | 8: 11915670-11916076 | 8: 12523080-12523500 |
| 8: 13265382-13265764 | 8: 13607807-13608190 | 8: 13608056-13608476 |
| 8: 17648803-17649153 | 8: 18097468-18097970 | 8: 18352237-18352571 |
| 8: 19010041-19010395 | 8: 19316645-19316956 | 8: 22120951-22121371 |
| 8: 22586809-22587229 | 8: 22600264-22600684 | 8: 22689742-22690162 |
| 8: 22690146-22690782 | 8: 22690940-22691360 | 8: 22703486-22703955 |
| 8: 22996796-22997216 | 8: 23679097-23679417 | 8: 23705022-23705519 |
| 8: 23707419-23707839 | 8: 23710166-23710586 | 8: 26047078-26047498 |
| 8: 26267135-26267625 | 8: 26864550-26864970 | 8: 27634345-27634765 |
| 8: 29348050-29348470 | 8: 30028407-30028827 | 8: 33564855-33565288 |
| 8: 37479387-37479807 | 8: 37546761-37547189 | 8: 37758281-37758697 |
| 8: 37841174-37841594 | 8: 38057000-38057420 | 8: 38899998-38900418 |
| 8: 38936841-38937261 | 8: 39852259-39852751 | 8: 41306763-41307450 |
| 8: 41541670-41542090 | 8: 41566673-41567555 | 8: 41630856-41631276 |
| 8: 41665052-41665442 | 8: 41797765-41798298 | 8: 41897729-41898150 |
| 8: 42052645-42053065 | 8: 42658912-42659332 | 8: 43247129-43247549 |
| 8: 43271110-43271530 | 8: 46954862-46955286 | 8: 47889183-47889603 |
| 8: 47968595-47968994 | 8: 48274039-48274499 | 8: 51408058-51408478 |
| 8: 54089907-54090364 | 8: 54277899-54278384 | 8: 54338920-54339317 |
| 8: 54523115-54523526 | 8: 54569794-54570201 | 8: 54620869-54621289 |
| 8: 55449151-55449571 | 8: 55449896-55450326 | 8: 55451319-55451739 |
| 8: 55451597-55452017 | 8: 55452218-55452638 | 8: 55524957-55525382 |
| 8: 55528638-55529086 | 8: 55939581-55940022 | 8: 55939868-55940288 |
| 8: 56050283-56050637 | 8: 56101552-56101973 | 8: 56121171-56121694 |
| 8: 56390721-56391094 | 8: 56477985-56478405 | 8: 57207805-57208225 |
| 8: 57210089-57210509 | 8: 57210633-57211053 | 8: 57212184-57212604 |
| 8: 57217495-57217915 | 8: 58145876-58146389 | 8: 59030336-59030683 |
| 8: 60264003-60264423 | 8: 60614353-60614760 | 8: 61135665-61136085 |
| 8: 62143920-62144326 | 8: 63145120-63145496 | 8: 63145562-63145970 |
| 8: 63323104-63323505 | 8: 63323296-63323714 | 8: 64378975-64379395 |
| 8: 65853513-65853894 | 8: 65864829-65865249 | 8: 65865365-65865785 |
| 8: 66049485-66049858 | 8: 66137655-66138135 | 8: 66439334-66439754 |
| 8: 66460112-66460532 | 8: 70535131-70535551 | 8: 72154363-72154916 |
| 8: 72286721-72287095 | 8: 73291721-73292141 | 8: 78508146-78508566 |
| 8: 80080314-80080780 | 8: 81630496-81631284 | 8: 84184765-84185282 |
| 8: 85178273-85178693 | 8: 85643792-85644212 | 8: 85655706-85656126 |
| 8: 86069272-86069692 | 8: 86069547-86069967 | 8: 86416956-86417376 |
| 8: 86661784-86662204 | 8: 87873855-87874275 | 8: 90853001-90853406 |
| 8: 91970571-91970991 | 8: 92144142-92144562 | 8: 93671272-93671664 |
| 8: 94945549-94945957 | 8: 95072781-95073669 | 8: 96333485-96333905 |
| 8: 99982479-99982799 | 8: 100073498-100073810 | 8: 100809369-100809789 |
| 8: 100900315-100900735 | 8: 101155978-101156398 | 8: 101223856-101224276 |
| 8: 102657609-102658029 | 8: 106060355-106060775 | 8: 106215558-106215978 |
| 8: 106274115-106274535 | 8: 106456081-106456462 | 8: 110832785-110833166 |
| 8: 113376950-113377370 | 8: 113884620-113885010 | 8: 115739502-115739872 |
| 8: 115804059-115804411 | 8: 116716490-116716910 | 8: 118044756-118045172 |
| 8: 119873005-119873425 | 8: 120125843-120126354 | 8: 120865917-120866272 |
| 8: 121502661-121503062 | 8: 122681275-122681643 | 8: 122875426-122875819 |
| 8: 126142974-126143372 | 8: 127482259-127482634 | 8: 127849511-127850128 |
| 8: 127918794-127919214 | 8: 128157655-128158006 | 8: 128859806-128860215 |
| 8: 128873535-128873889 | 8: 128876114-128876600 | 8: 128960699-128961119 |
| 8: 128969308-128969630 | 8: 129242042-129242461 | 8: 129273810-129274173 |
| 8: 129386774-129387134 | 8: 129542905-129543256 | 8: 129564999-129565415 |
| 8: 129628632-129629085 | 8: 129728664-129729084 | 8: 129819722-129820142 |
| 8: 130828927-130829332 | 8: 131934095-131934515 | 8: 132129169-132129769 |
| 8: 132319498-132319918 | 8: 133250323-133250728 | 8: 133293739-133294159 |
| 8: 133454723-133455143 | 8: 134478504-134478924 | 8: 135589592-135589996 |
| 8: 137843987-137844397 | 8: 138152176-138152606 | 8: 139618604-139619024 |
| 8: 139701977-139702397 | 8: 140596861-140597304 | 8: 141164340-141164776 |
| 8: 141175125-141175545 | 8: 141206355-141206775 | 8: 141265400-141265874 |
| 8: 141273138-141273558 | 8: 141619826-141620246 | 8: 141622200-141622620 |
| 8: 141760544-141760964 | 8: 141903017-141903515 | 8: 142200948-142201384 |
| 8: 142247894-142248314 | 8: 142248928-142249292 | 8: 142251864-142252284 |
| 8: 142479068-142479551 | 8: 142544242-142544662 | 8: 142613169-142613589 |
| 8: 142699739-142700159 | 8: 142710182-142710602 | 8: 142761731-142762151 |
| 8: 142834332-142834752 | 8: 142953506-142953926 | 8: 142985340-142985719 |
| 8: 142989321-142989741 | 8: 142989503-142989923 | 8: 143411131-143411551 |
| 8: 143529361-143529781 | 8: 143716248-143716668 | 8: 143770329-143770845 |
| 8: 144042587-144042988 | 8: 144048159-144048579 | 8: 144049308-144049844 |
| 8: 144049820-144050352 | 8: 144050232-144051051 | 8: 144051900-144052320 |
| 8: 144083873-144084293 | 8: 144335850-144336270 | 8: 144354674-144355094 |
| 8: 144518873-144519274 | 8: 144527994-144528414 | 8: 144699872-144700292 |
| 8: 145069298-145069718 | 9: 72612-73059 | 9: 456295-456707 |
| 9: 708216-708636 | 9: 842826-843263 | 9: 971142-971562 |
| 9: 977620-978100 | 9: 1045848-1046367 | 9: 1459775-1460160 |
| 9: 2157529-2157949 | 9: 2158154-2158643 | 9: 3441341-3441810 |
| 9: 4803868-4804288 | 9: 5185535-5185955 | 9: 6565306-6565701 |
| 9: 6925019-6925391 | 9: 7962005-7962421 | 9: 9090911-9091331 |
| 9: 11618084-11618504 | 9: 14346406-14346826 | 9: 14691141-14691623 |
| 9: 15401375-15401791 | 9: 16522154-16522574 | 9: 16828976-16829396 |
| 9: 18260554-18261001 | 9: 18976488-18977010 | 9: 19184323-19184743 |
| 9: 22005052-22005472 | 9: 26801553-26801976 | 9: 30799667-30800087 |
| 9: 32333656-32334076 | 9: 32631302-32631698 | 9: 33871418-33871884 |
| 9: 34380089-34380509 | 9: 34623359-34623779 | 9: 34623733-34624284 |
| 9: 34624478-34624898 | 9: 35908149-35908569 | 9: 37034901-37035321 |
| 9: 37037953-37038494 | 9: 37229860-37230171 | 9: 37239219-37239637 |
| 9: 37327452-37327864 | 9: 42743164-42743584 | 9: 42972458-42972878 |
| 9: 62799967-62800387 | 9: 63876301-63876711 | 9: 64070121-64070541 |
| 9: 65467144-65467564 | 9: 66153986-66154406 | 9: 66831328-66831748 |
| 9: 68304170-68304595 | 9: 68307018-68307477 | 9: 68357132-68357617 |
| 9: 68895867-68896287 | 9: 69086904-69087320 | 9: 71984918-71985338 |
| 9: 76996564-76996984 | 9: 77019375-77019989 | 9: 77647390-77648385 |
| 9: 79668287-79668707 | 9: 79975849-79976283 | 9: 83894435-83894783 |
| 9: 86036411-86036990 | 9: 87451308-87451728 | 9: 88532222-88532773 |
| 9: 88578081-88578501 | 9: 88991944-88992364 | 9: 89649032-89649452 |
| 9: 89701158-89701739 | 9: 90226326-90226716 | 9: 90227432-90227852 |
| 9: 91117887-91118588 | 9: 91844126-91844627 | 9: 91896758-91897178 |
| 9: 92807091-92807511 | 9: 93860678-93861098 | 9: 93959608-93960028 |
| 9: 94908942-94909322 | 9: 96320729-96321204 | 9: 96719354-96720134 |
| 9: 97076797-97077371 | 9: 97854892-97855377 | 9: 98157602-98157934 |
| 9: 98775891-98776311 | 9: 99223112-99223497 | 9: 105319120-105319542 |
| 9: 105522249-105522669 | 9: 108762206-108762659 | 9: 110078765-110079152 |
| 9: 112308771-112309187 | 9: 112396819-112397178 | 9: 112870203-112870608 |
| 9: 113059675-113060171 | 9: 113581753-113582230 | 9: 114049616-114050036 |
| 9: 114558753-114559173 | 9: 116156992-116157445 | 9: 120448065-120448513 |
| 9: 121286227-121286708 | 9: 121289244-121289721 | 9: 121556191-121556565 |
| 9: 121557621-121558041 | 9: 121698521-121698971 | 9: 121736084-121736504 |
| 9: 121736532-121737014 | 9: 121799007-121799427 | 9: 122222862-122223282 |
| 9: 124016213-124016931 | 9: 124020881-124021525 | 9: 124291650-124292070 |
| 9: 124502812-124503435 | 9: 125003465-125003872 | 9: 127519345-127519765 |
| 9: 127749204-127749624 | 9: 127913009-127913463 | 9: 128022108-128022479 |
| 9: 128339693-128340113 | 9: 128624645-128625065 | 9: 128851598-128851951 |
| 9: 129225792-129226212 | 9: 129282327-129282747 | 9: 129336929-129337479 |
| 9: 129352273-129352753 | 9: 129459161-129459588 | 9: 129460876-129461296 |
| 9: 129664723-129665228 | 9: 129777044-129777747 | 9: 130240903-130241244 |
| 9: 130285096-130285516 | 9: 130535689-130536216 | 9: 130662648-130663068 |
| 9: 130740325-130740745 | 9: 131238596-131239016 | 9: 131656750-131657170 |
| 9: 132201479-132201899 | 9: 132201625-132202045 | 9: 132381699-132382052 |
| 9: 132482305-132482725 | 9: 132582666-132583086 | 9: 132583083-132583503 |
| 9: 132644658-132645078 | 9: 132973055-132973475 | 9: 133050764-133051184 |
| 9: 133163936-133164426 | 9: 133200441-133200861 | 9: 133225295-133225715 |
| 9: 133230493-133230913 | 9: 133274268-133275038 | 9: 133357588-133357996 |
| 9: 133531762-133532182 | 9: 133651411-133651814 | 9: 133862784-133863204 |
| 9: 134222955-134223357 | 9: 134344212-134344632 | 9: 134372825-134373245 |
| 9: 134391152-134391844 | 9: 134448003-134448375 | 9: 134557248-134557668 |
| 9: 134564367-134564969 | 9: 134658004-134658424 | 9: 134728418-134728838 |
| 9: 134731197-134731672 | 9: 134765697-134766117 | 9: 134786013-134786433 |
| 9: 134792943-134793363 | 9: 134845494-134845859 | 9: 135438225-135438645 |
| 9: 135544107-135544527 | 9: 135580694-135581114 | 9: 135680337-135680757 |
| 9: 135744853-135745273 | 9: 135764918-135765338 | 9: 135827600-135828020 |
| 9: 135980904-135981324 | 9: 136050972-136051406 | 9: 136199665-136200085 |
| 9: 136395580-136396132 | 9: 136533717-136534206 | 9: 136538687-136539145 |
| 9: 136587070-136587490 | 9: 136659171-136659591 | 9: 136695915-136696382 |
| 9: 136821182-136821602 | 9: 136840407-136840930 | 9: 136845930-136846350 |
| 9: 137269580-137270000 | 9: 137411619-137412045 | 9: 137436185-137436597 |
| 9: 137485347-137485819 | 9: 137559101-137559513 | 9: 137613888-137614308 |
| 9: 137715333-137715753 | 9: 137744775-137745195 | 9: 137795124-137795544 |
| 9: 137921102-137921522 | 9: 138120148-138120568 | |

### Table 6A—PrCa (CaNCaP)

The DMRs may be those disclosed at: https://zenodo.org/doi/10.5281/zenodo.10013535 as of 17 Oct. 2023. The entire contents of https://zenodo.org/doi/10.5281/zenodo.10013535 are expressly incorporated herein by reference as “Table 6A”.

### REFERENCES

1. Gale D, Lawson A R J, Howarth K, Madi M, Durham B, Smalley S, Calaway J, Blais S, Jones G, Clark J, Dimitrov P, Pugh M, Woodhouse S, Epstein M, Fernandez-Gonzalez A, Whale A S, Huggett J F, Foy C A, Jones G M, Raveh-Amit H, Schmitt K, Devonshire A, Green E, Forshew T, Plagnol V, Rosenfeld N. Development of a highly sensitive liquid biopsy platform to detect clinically-relevant cancer mutations at low allele fractions in cell-free DNA. PLOS One. 2018; 13 (3): e0194630.
2. Douillard J Y, Ostoros G, Cobo M, Ciuleanu T, Cole R, McWalter G, Walker J, Dearden S, Webster A, Milenkova T, McCormack R. Gefitinib treatment in EGFR mutated caucasian NSCLC: circulating-free tumor DNA as a surrogate for determination of EGFR status. J Thorac Oncol. 2014; 9 (9): 1345-53.
3. Schwaederle M C, Patel S P, Husain H, Ikeda M, Lanman R B, Banks K C, Talasaz A, Bazhenova L, Kurzrock R. Utility of Genomic Assessment of Blood-Derived Circulating Tumor DNA (ctDNA) in Patients with Advanced Lung Adenocarcinoma. Clin Cancer Res. 2017; 23 (17): 5101-5111.
4. Coombes R C, Page K, Salari R, Hastings R K, Armstrong A, Ahmed S, Ali S, Cleator S, Kenny L, Stebbing J, Rutherford M, Sethi H, Boydell A, Swenerton R, Fernandez-Garcia D, Gleason K L I, Goddard K, Guttery D S, Assaf Z J, Wu H T, Natarajan P, Moore D A, Primrose L, Dashner S, Tin A S, Balcioglu M, Srinivasan R, Shchegrova S V, Olson A, Hafez D, Billings P, Aleshin A, Rehman F, Toghill B J, Hills A, Louie M C, Lin C J, Zimmermann B G, Shaw J A. Personalized Detection of Circulating Tumor DNA Antedates Breast Cancer Metastatic Recurrence. Clin Cancer Res. 2019; 25 (14): 4255-4263.
5. Tie J, Wang Y, Tomasetti C, Li L, Springer S, Kinde I, Silliman N, Tacey M, Wong H L, Christie M, Kosmider S, Skinner I, Wong R, Steel M, Tran B, Desai J, Jones I, Haydon A, Hayes T, Price T J, Strausberg R L, Diaz L A Jr, Papadopoulos N, Kinzler K W, Vogelstein B, Gibbs P. Circulating tumor DNA analysis detects minimal residual disease and predicts recurrence in patients with stage II colon cancer. Sci Transl Med. 2016; 8 (346): 346ra92.
6. Garcia-Murillas I, Schiavon G, Weigelt B, Ng C, Hrebien S, Cutts R J, Cheang M, Osin P, Nerurkar A, Kozarewa I, Garrido J A, Dowsett M, Reis-Filho J S, Smith I E, Turner N C. Mutation tracking in circulating tumor DNA predicts relapse in early breast cancer. Sci Transl Med. 2015; 7 (302): 302ra133.
7. Bettegowda C, Sausen M, Leary R J, Kinde I, Wang Y, Agrawal N, Bartlett B R, Wang H, Luber B, Alani R M, Antonarakis E S, Azad N S, Bardelli A, Brem H, Cameron J L, Lee C C, Fecher L A, Gallia G L, Gibbs P, Le D, Giuntoli R L, Goggins M, Hogarty M D, Holdhoff M, Hong S M, Jiao Y, Juhl H H, Kim J J, Siravegna G, Laheru D A, Lauricella C, Lim M, Lipson E J, Marie S K, Netto G J, Oliner K S, Olivi A, Olsson L, Riggins G J, Sartore-Bianchi A, Schmidt K, Shih I M, Oba-Shinjo S M, Siena S, Theodorescu D, Tie J, Harkins T T, Veronese S, Wang T L, Weingart J D, Wolfgang C L, Wood L D, Xing D, Hruban R H, Wu J, Allen P J, Schmidt C M, Choti M A, Velculescu V E, Kinzler K W, Vogelstein B, Papadopoulos N, Diaz L A Jr. Detection of circulating tumor DNA in early- and late-stage human malignancies. Sci Transl Med. 2014; 6 (224): 224ra24.
8. Cohen J D, Li L, Wang Y, Thoburn C, Afsari B, Danilova L, Douville C, Javed A A, Wong F, Mattox A, Hruban R H, Wolfgang C L, Goggins M G, Dal Molin M, Wang T L, Roden R, Klein A P, Ptak J, Dobbyn L, Schaefer J, Silliman N, Popoli M, Vogelstein J T, Browne J D, Schoen R E, Brand R E, Tie J, Gibbs P, Wong H L, Mansfield A S, Jen J, Hanash S M, Falconi M, Allen P J, Zhou S, Bettegowda C, Diaz L A Jr, Tomasetti C, Kinzler K W, Vogelstein B, Lennon A M, Papadopoulos N. Detection and localization of surgically resectable cancers with a multi-analyte blood test. Science. 2018; 359 (6378): 926-930.
9. Phallen J, Sausen M, Adleff V, Leal A, Hruban C, White J, Anagnostou V, Fiksel J, Cristiano S, Papp E, Speir S, Reinert I, Orntoft M W, Woodward B D, Murphy D, Parpart-Li S, Riley D, Nesselbush M, Sengamalay N, Georgiadis A, Li Q K, Madsen M R, Mortensen F V, Huiskens J, Punt C, van Grieken N, Fijneman R, Meijer G, Husain H, Scharpf R B, Diaz L A Jr, Jones S, Angiuoli S, Ørntoft T, Nielsen H J, Andersen C L, Velculescu V E. Direct detection of early-stage cancers using circulating tumor DNA. Sci Transl Med. 2017; 9 (403).
10. Newman A M, Bratman S V, To J, Wynne J F, Eclov N C, Modlin L A, Liu C L, Neal J W, Wakelee H A, Merritt R E, Shrager J B, Loo B W Jr, Alizadeh A A, Diehn M. An ultrasensitive method for quantitating circulating tumor DNA with broad patient coverage. Nat Med. 2014; 20 (5): 548-54.
11. Wan J C M, Massie C, Garcia-Corbacho J, Mouliere F, Brenton J D, Caldas C, Pacey S, Baird R, Rosenfeld N. Liquid biopsies come of age: towards implementation of circulating tumour DNA. Nat Rev Cancer. 2017; 17 (4): 223-238.
12. Wan J C M, Heider K, Gale D, Murphy S, Fisher E, Morris J, Mouliere F, Chandrananda D, Marshall A, Gill A B, Chan P Y, Barker E, Young G, Cooper W N, Hudecova I, Marass F, Bignell G R, Alifrangis C, Middleton M R, Gallagher F A, Parkinson C, Durrani A, McDermott U, Smith C G, Massie C, Corrie P G, Rosenfeld N. High-sensitivity monitoring of ctDNA by patient-specific sequencing panels and integration of variant reads. biorxiv 2019; 759399; doi: doi.org/10.1101/759399
13. Beaver J A, Jelovac D, Balukrishna S, Cochran R, Croessmann S, Zabransky D J, Wong H Y, Toro P V, Cidado J, Blair B G, Chu D, Burns T, Higgins M J, Stearns V, Jacobs L, Habibi M, Lange J, Hurley P J, Lauring J, VanDenBerg D, Kessler J, Jeter S, Samuels M L, Maar D, Cope L, Cimino-Mathews A, Argani P, Wolff A C, Park B H. Detection of cancer DNA in plasma of patients with early-stage breast cancer. Clin Cancer Res. 2014; 20 (10): 2643-2650.
14. Newman A M, Lovejoy A F, Klass D M, Kurtz D M, Chabon J J, Scherer F, Stehr H, Liu C L, Bratman S V, Say C, Zhou L, Carter J N, West R B, Sledge G W, Shrager J B, Loo B W Jr, Neal J W, Wakelee H A, Diehn M, Alizadeh A A. Integrated digital error suppression for improved detection of circulating tumor DNA. Nat Biotechnol. 2016; 34 (5): 547-555.
15. Bird A P. The relationship of DNA methylation to cancer. Cancer Surv. 1996; 28:87-101.
16. Laird P W. The power and the promise of DNA methylation markers. Nat Rev Cancer. 2003; 3 (4): 253-66.
17. Massie C E, Mills I G, Lynch A G. The importance of DNA methylation in prostate cancer development. J Steroid Biochem Mol Biol. 2017; 166:1-15.
18. Chan K C, Jiang P, Chan C W, Sun K, Wong J, Hui E P, Chan S L, Chan W C, Hui D S, Ng S S, Chan H L, Wong C S, Ma B B, Chan A T, Lai P B, Sun H, Chiu R W, Lo Y M. Noninvasive detection of cancer-associated genome-wide hypomethylation and copy number aberrations by plasma DNA bisulfite sequencing. Proc Natl Acad Sci USA. 2013; 110 (47): 18761-8.
19. Lehmann-Werman R, Neiman D, Zemmour H, Moss J, Magenheim J, Vaknin-Dembinsky A, Rubertsson S, Nellgård B, Blennow K, Zetterberg H, Spalding K, Haller M J, Wasserfall C H, Schatz D A, Greenbaum C J, Dorrell C, Grompe M, Zick A, Hubert A, Maoz M, Fendrich V, Bartsch D K, Golan T, Ben Sasson S A, Zamir G, Razin A, Cedar H, Shapiro A M, Glaser B, Shemer R, Dor Y. Identification of tissue-specific cell death using methylation patterns of circulating DNA. Proc Natl Acad Sci USA. 2016; 113 (13): E1826-34.
20. Moss J, Magenheim J, Neiman D, Zemmour H, Loyfer N, Korach A, Samet Y, Maoz M, Druid H, Arner P, Fu K Y, Kiss E, Spalding K L, Landesberg G, Zick A, Grinshpun A, Shapiro AMJ, Grompe M, Wittenberg A D, Glaser B, Shemer R, Kaplan T, Dor Y. Comprehensive human cell-type methylation atlas reveals origins of circulating cell-free DNA in health and disease. Nat Commun. 2018; 9 (1): 5068.
21. Goessl C, Krause H, Müller M, Heicappell R, Schrader M, Sachsinger J, Miller K. Fluorescent methylation-specific polymerase chain reaction for DNA-based detection of prostate cancer in bodily fluids. Cancer Res. 2000; 60 (21): 5941-5.
22. Vener T, Derecho C, Baden J, Wang H, Rajpurohit Y, Skelton J, Mehrotra J, Varde S, Chowdary D, Stallings W, Leibovich B, Robin H, Pelzer A, Schäfer G, Auprich M, Mannweiler S, Amersdorfer P, Mazumder A. Development of a multiplexed urine assay for prostate cancer diagnosis. Clin Chem. 2008; 54 (5): 874-82.
23. O'Reilly E, Tuzova A V, Walsh A L, Russell N M, O'Brien O, Kelly S, Dhomhnallain O N, DeBarra L, Dale C M, Brugman R, Clarke G, Schmidt O, O'Meachair S, Patil D, Pellegrini K L, Fleshner N, Garcia J, Zhao F, Finn S, Mills R, Hanna M Y, Hurst R, MCEvoy E, Gallagher W M, Manecksha R P, Cooper C S, Brewer D S, Bapat B, Sanda M G, Clark J, Perry A S. epiCapture: A Urine DNA Methylation Test for Early Detection of Aggressive Prostate Cancer. JCO Precis Oncol. 2019.
24 Cheng T H T, Jiang P, Teoh J Y C, Heung M M S, Tam J C W, Sun X, Lee W S, Ni M, Chan R C K, Ng C F, Chan K C A, Chiu R W K, Lo Y M D. Noninvasive Detection of Bladder Cancer by Shallow-Depth Genome-Wide Bisulfite Sequencing of Urinary Cell-Free DNA for Methylation and Copy Number Profiling. Clin Chem. 2019; 65 (7): 927-936.
25. Berman B P, Weisenberger D J, Aman J F, Hinoue T, Ramjan Z, Liu Y, Noushmehr H, Lange C P, van Dijk C M, Tollenaar R A, Van Den Berg D, Laird P W. Regions of focal DNA hypermethylation and long-range hypomethylation in colorectal cancer coincide with nuclear lamina-associated domains. Nat Genet. 2011; 44 (1): 40-6
26. Shen S Y, Singhania R, Fehringer G, Chakravarthy A, Roehrl MHA, Chadwick D, Zuzarte P C, Borgida A, Wang T T, Li T, Kis O, Zhao Z, Spreafico A, Medina TDS, Wang Y, Roulois D, Ettayebi I, Chen Z, Chow S, Murphy T, Arruda A, O'Kane G M, Liu J, Mansour M, McPherson J D, O'Brien C, Leighl N, Bedard P L, Fleshner N, Liu G, Minden M D, Gallinger S, Goldenberg A, Pugh T J, Hoffman M M, Bratman S V, Hung R J, De Carvalho D D. Sensitive tumour detection and classification using plasma cell-free DNA methylomes. Nature. 2018; 563 (7732): 579-583.
27. Song C X, Yin S, Ma L, Wheeler A, Chen Y, Zhang Y, Liu B, Xiong J, Zhang W, Hu J, Zhou Z, Dong B, Tian Z, Jeffrey S S, Chua M S, So S, Li W, Wei Y, Diao J, Xie D, Quake S R. 5-Hydroxymethylcytosine signatures in cell-free DNA provide information about tumor types and stages. Cell Res. 2017; 27 (10): 1231-1242.
28. Li W, Zhang X, Lu X, You L, Song Y, Luo Z, Zhang J, Nie J, Zheng W, Xu D, Wang Y, Dong Y, Yu S, Hong J, Shi J, Hao H, Luo F, Hua L, Wang P, Qian X, Yuan F, Wei L, Cui M, Zhang T, Liao Q, Dai M, Liu Z, Chen G, Meckel K, Adhikari S, Jia G, Bissonnette M B, Zhang X, Zhao Zhang W, Y, He C, Liu J. 5-Hydroxymethylcytosine signatures in circulating cell-free DNA as diagnostic biomarkers for human cancers. Cell Res. 2017; 27 (10): 1243-1257.
29. Klein E A, Hubbell E, Maddala T, Aravanis A, Beausang J F, Filippova D, Gross S, Jamshidi A, Kurtzman K, Shen L, Valouev A, Venn O, Zhang N, Smith D A, Yeatman T J, Tibshirani R, Williams R T, Hartman A R, Seiden M, Liu M C. Development of a comprehensive cell-free DNA (cfDNA) assay for early detection of multiple tumor types: The Circulating Cell-free Genome Atlas (CCGA) study. J Clin OncoL 36, no. 15_suppl 2018; 12021-12021.
30. Liu M C, Jamshidi A, Venn O, Fields A P, Maher M C, Cann G, Amini H, Gross S, Bredno J, Miller M, Schellenberger J, Kurtzman K N, Fung E T, Maddala T, Oxnard G R, Klein E A, Spigel D R, Hartman A R, Aravanis A, Seiden M. Genome-wide cell-free DNA (cfDNA) methylation signatures and effect on tissue of origin (TOO) performance. J Clin Oncol 37, 2019 (suppl; abstr 3049).
31. Cancer Genome Atlas Research Network. The Molecular Taxonomy of Primary Prostate Cancer. Cell. 2015 Nov. 5; 163 (4): 1011-25.
32. Massie C E, Spiteri I, Ross-Adams H, Luxton H, Kay J, Whitaker H C, Dunning M J, Lamb A D, Ramos-Montoya A, Brewer D S, Cooper C S, Eeles R; U K Prostate ICGC Group, Warren A Y, Tavare S, Neal D E, Lynch A G. HES5 silencing is an early and recurrent change in prostate tumourigenesis. Endocr Relat Cancer. 2015; 22 (2): 131-44.

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Any sub-titles herein are included for convenience only, and are not to be construed as limiting the disclosure in any way.

1. A method of analysing a sample obtained from a subject having or suspected of having a somatic disease state, the method comprising:
(a) providing a cell-free DNA (cfDNA)-containing sample obtained from the subject and performing methylation sequencing of at least 10, 25, 50 or at least 100 predetermined genomic regions;
(b) generating metrics from the methylation sequencing reads in step (a) that represent one or both of (i) a measure of the degree to which the methylation observed within a sequencing read is similar to a previously determined disease methylation signal, said measure comprising a read rating generated by providing a vector of methylation states for each methylation sequencing read and summing the information content of all methylation states that are concordant with the methylation state for the same region previously determined in a one or more disease samples; and (ii) a measure of intra-read methylation heterogeneity; and
(c) integrating the metrics generated in step (b) across the at least 10, 25, 50 or at least 100 predetermined genomic regions to obtain a global measure of disease-originating cfDNA in the sample obtained from the subject.

2. The method of claim 1, wherein said methylation sequencing comprises a conversion step or comprises single molecule DNA sequencing, optionally wherein said methylation sequencing comprises a non-destructive, enzyme-based conversion method to differentiate unmethylated cytosine from 5-methylcytosine and/or 5-hydroxymethylcytosine.

3. (canceled)

4. The method of claim 1, wherein the somatic disease state is a cancer and wherein the global measure of disease-originating cfDNA in the sample is a measure of circulating tumour DNA (ctDNA) in the sample obtained from the subject.

5. The method of claim 1, wherein each of said at least 10 predetermined genomic regions satisfies at least two of the following conditions:
(i) the region comprises a differentially methylated cytosine (DMC) or differentially methylated region (DMR) defined by the methylation status of one or more disease-state specific CpG, wherein said DMC or DMR exhibits a significance threshold of q<0.05 or q<0.01 for high-burden disease sample (e.g. cancer tissue, proximal sample or high disease burden cfDNA sample) versus healthy control tissue as determined by differential methylation analysis;
(ii) the region exhibits a difference in methylation between healthy cfDNA samples and disease tissue (e.g. cancer tissue) within the range 0.6 to 1.0, optionally wherein the difference is determined according to the formula |betaₚₗₐₛₘₐ−round(betaₜᵢₛₛᵤₑ)|>0.6, wherein betaₚₗₐₛₘₐ is the methylation signal as a proportion on a 0-1 scale (0 being fully unmethylated and 1 being fully methylated at a given CpG position); and round is a function that limits the number of significant digits to zero for the beta value from tissue data, resulting in a binary classification of disease state derived methylation markers as either hypomethylated (rounded beta equal to 0) or hypermethylated (rounded beta equal to 1); and
(iii) the region comprises at least 2 CpGs.

6. The method of claim 1, wherein said at least 10 predetermined genomic regions comprises at least 500, at least 1000, at least 5000, or at least 10000 predetermined genomic regions.

7. The method of claim 1, wherein said read rating is generated by providing a vector of methylation states for each methylation sequencing read and summing the information content of all methylation states that are concordant with the methylation state for the same region previously determined in a consensus of 50 or more disease tissue samples or other high burden disease samples (e.g. 50 or more cancer samples), optionally wherein said read rating is a penalised read rating, the generation of which further comprises subtracting the information content of all of the methylation states opposite to the methylation state for the same region previously determined in the one or more disease samples (e.g. one or more cancer samples).

8. (canceled)

9. The method of claim 7, wherein the read rating for each sequencing read is compared with a read rating threshold to determine whether the read is a disease-specific read (e.g. a cancer-specific read), optionally after correcting for background methylation for the same region in samples from healthy controls, optionally wherein the read rating threshold is a constant value or is a statistical measure of variation from a distribution of read ratings observed for the same region in healthy controls, further optionally wherein the read rating threshold is 4.32 Shannons or is the 99th quantile.

10-11. (canceled)

12. The method of claim 9, wherein integrating the metrics generated in step (b) across the at least 10 predetermined genomic regions to obtain the global measure comprises summing all the read ratings for all sequencing reads that pass the read rating threshold to be determined to be disease-specific reads (e.g. cancer-specific reads).

13. The method of claim 1, wherein said metric, that measures intra-read methylation heterogeneity, comprises a simple methylation uniformity gauge (SMUGE) score generated by providing a vector \vec{rₖ} of methylation states for each methylation sequencing read, wherein
$$\vec{{r}_{k}} = [ {r}_{1} , … , {r}_{j} ] , where {r}_{i} ∈ ( - 1 , 1 )$$
where −1 is unmethylated cytosine and 1 is methylated cytosine, j is the number of methylation sites in the predetermined genomic region
and wherein the SMUGE score for the k^{th} read is determined according to the formula:
$${smuge}_{k} = {\sum }_{j}^{J - 1} \frac{\vert {r}_{j} - {r}_{j + 1} \vert}{2}$$

14. The method of claim 13, wherein integrating the metrics generated in step (b) across the at least 10 predetermined genomic regions to obtain the global measure comprises summing or averaging the SMUGE scores for all sequencing reads that pass the SMUGE threshold to be determined as disease-specific reads (e.g. cancer-specific reads).

15. The method of claim 1, wherein the metrics that represent (i) the measure of the degree to which the methylation observed within a sequencing read is similar to a previously determined cancer methylation signal; and (ii) the measure of intra-read methylation heterogeneity are combined, optionally wherein (ii) the measure of intra-read methylation heterogeneity is used to classify reads that cannot be unequivocally classified using metric (i) alone.

16. The method of claim 1, wherein the cell-free DNA (cfDNA)-containing sample comprises 1 μg to 200 ng of double stranded cfDNA and/or wherein the cfDNA sample comprises less than 1 million, less than 100,000, less than 10,000, less than 1,000 or less than 100 haploid human genome equivalents.

17. The method of claim 1, wherein said non-destructive, enzyme-based conversion method comprises ten-eleven translocation dioxygenase 2 (TET2)-based conversion followed by a bead-based clean-up step of the TET2-converted DNA in which the bead ratio is selected to enhance recovery of short ctDNA fragments.

18. The method of claim 1, wherein said methylation sequencing comprises targeted capture sequencing, which comprises a preceding step of PCR amplification of the post-conversion cfDNA using uracil base-tolerant PCR reagents, optionally wherein said targeting capture sequencing comprises a target enrichment step that employs one or more probe panels that target said at least 10 predetermined genomic regions, further optionally wherein said one or more probe panels target at least 10, at least 25, at least 50, at least 100, at least 1000 or at least 5000 genomic regions set forth in any one of Tables 1 to 4 or 6 or any one of Tables 1A, 2A, 3A, 4A and 6A.

19-20. (canceled)

21. The method of claim 1, wherein the method comprises a step, preceding said methylation sequencing, in which at least one biotinylated adaptor is ligated to cfDNA in said cfDNA-containing sample.

22. The method of claim 1, wherein the method:
(a) comprises detecting and/or staging a cancer, monitoring cancer progression or remission, or monitoring a response to an anti-cancer therapy in said subject,
(b) comprises detecting and/or staging a cancer, optionally wherein the cancer is prostate cancer, clear cell renal cell carcinoma, glioblastoma or oesophageal carcinoma;
(c) comprises monitoring the treatment of the subject, wherein the subject is being treated with a tyrosine kinase inhibitor (TKI), a poly ADP ribose polymerase (PARP) inhibitor and/or a surgical treatment;
(d) comprises predicting response of the subject to an anti-cancer therapeutic, wherein the subject has cancer, optionally wherein the anti-cancer therapeutic comprises Cabozantinib, Lenvatinib+Everolimus, Pazopanib and/or Tivozanib;
(e) comprises predicting the survival time of the subject, wherein the subject has cancer, optionally wherein the cancer comprises prostate cancer; and/or
(f) is carried out on at least one sample obtained from each of a plurality of subjects having cancer and wherein the method stratifies the subjects according to the stage or severity of the cancer.

23-27. (canceled)

28. The method of claim 1, wherein the sample comprises a tissue sample, a proximal sample or a distal minimally invasive samples, such as a blood plasma sample, a blood sample, a urine sample, a seminal fluid sample, a saliva sample, a cerebrospinal fluid (CSF) sample, an amniotic fluid sample, a fine-needle aspirate sample or a proximal tissue sample comprising fluid and/or cellular material.

29. The method of claim 1, wherein the somatic disease state is:
(A) prostate cancer (PrCa), optionally early stage (localised PrCa), late stage (locally advanced PrCa) or metastatic PrCa, optionally wherein the at least 10 predetermined genomic regions are selected from the genomic regions set forth in Table 4 or Table 4A or Table 6 or Table 6A, further optionally wherein the predetermined genomic regions comprise at least 10%, 25%, 50%, 75% or at least 90% of the genomic regions set forth in Table 4 or Table 4A or Table 6 or Table 6A;
(B) oesophageal carcinoma (OAC), optionally wherein the at least 10 predetermined genomic regions are selected from the genomic regions set forth in Table 3 or Table 3A, further optionally wherein the predetermined genomic regions comprise at least 10%, 25%, 50%, 75% or at least 90% of the genomic regions set forth in Table 3 or Table 3A;
(C) glioblastoma (GBM), optionally wherein the at least 10 predetermined genomic regions are selected from the genomic regions set forth in Table 2 or Table 2A, further optionally wherein the predetermined genomic regions comprise at least 10%, 25%, 50%, 75% or at least 90% of the genomic regions set forth in Table 2 or Table 2A; or
(D) clear cell renal cell carcinoma (ccRCC), optionally, wherein the at least 10 predetermined genomic regions are selected from the genomic regions set forth in Table 1 or Table 1A, further optionally wherein the predetermined genomic regions comprise at least 10%, 25%, 50%, 75% or at least 90% of the genomic regions set forth in Table 1 or Table 1A.

30-40. (canceled)

41. A computer-implemented method for predicting whether a cell-free DNA (cfDNA)-containing sample obtained from a subject contains DNA originating from a somatic disease state tissue, the method comprising:
(a) providing methylation sequencing data representing methylation sequencing reads of cfDNA from the sample obtained from the subject, which reads cover at least 10, 25, 50 or at least 100 predetermined genomic regions;
(b) generating metrics from the methylation sequencing data in step (a) that represent one or both of (i) a measure of the degree to which the methylation observed within a sequencing read is similar to a previously determined disease methylation signal, said measure comprising a read rating generated by providing a vector of methylation states for each methylation sequencing read and summing the information content of all methylation states that are concordant with the methylation state for the same region previously determined in a one or more disease samples; and (ii) a measure of intra-read methylation heterogeneity; and
(c) integrating the metrics generated in step (b) across the at least 10, 25, 50 or at least 100 predetermined genomic regions to obtain a global measure of disease-originating cfDNA in the sample obtained from the subject,
wherein the sample is predicted to contain disease-originating cfDNA if the global measure of disease-originating cfDNA is greater than a background threshold

42. (canceled)

43. A method of treatment of a subject suspect of having a cancer, comprising:
performing the method of claim 1 on a cell free DNA (cfDNA)-containing sample obtained from the subject, wherein the sample is determined to contain circulating tumour DNA (ctDNA) thereby indicating that the subject does have a cancer;
administering treatment with curative intent using surgery, radiotherapy, chemotherapy, targeted therapy or immunotherapy; administering salvage therapy using surgery and/or radiotherapy; administering a minimum therapeutically effective amount of a chemotherapeutic agent, targeted therapy, immunotherapy and/or radiotherapy to the subject.
